(19) United States
(12) Patent Application Publication
(10) Pub. No.: US 2026/0184697 A1
(43) Pub. Date: Jul. 2, 2026

(54) EZH1/2 INHIBITOR, PREPARATION THEREOF, AND USE THEREOF IN ANTI-TUMOR THERAPY

(71) Applicant: JIANGSU TASLY DIYI PHARMACEUTICAL CO., LTD., Jiangsu (CN)

(72) Inventors: Kailin HAN, Jiangsu (CN); Xiaohui MA, Jiangsu (CN); Jiawen LI, Jiangsu (CN); Zhifu WAN, Jiangsu (CN); Xiaotao WANG, Jiangsu (CN); Liming DONG, Jiangsu (CN); Hai TANG, Jiangsu (CN); Shuiping ZHOU, Jiangsu (CN); Jinyong CAI, Jiangsu (CN)

(21) Appl. No.: 18/727,876

(22) PCT Filed: Dec. 15, 2022
(86) PCT No.: PCT/CN2022/139254

§ 371 Date: Jul. 10, 2024

(30) Foreign Application Priority Data
Jan. 27, 2022 (CN) 202210105673.8

(51) Int. Cl. C07D 405/14; A61K 31/357; A61K 31/443; A61K 31/4433; A61K 31/4439; A61K 31/4545; A61K 31/496; A61K 31/5377; A61P 35/00; C07B 59/00; C07D 405/12; C07D 407/12; C07D 491/048; C07D 491/107

(52) U.S. Cl. CPC C07D 405/14; A61K 31/357; A61K 31/443; A61K 31/4433; A61K 31/4439; A61K 31/4545; A61K 31/496; A61K 31/5377; A61P 35/00; C07B 59/002; C07D 405/12; C07D 407/12; C07D 491/048; C07D 491/107

(57) ABSTRACT

The present invention relates to an EZH1/2 inhibitor, a preparation thereof, and a use thereof in anti-tumor therapy, specifically comprising a compound represented by formula I or formula II, or a deuterated compound, or a stereoisomer, or a pharmaceutically acceptable salt thereof, and further comprising a pharmaceutical composition of the compound, and a use of the foregoing as an EZH1/2 inhibitor in the preparation of medication for treating related diseases.

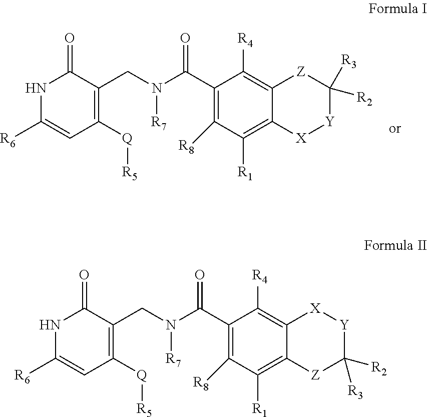

### TECHNICAL FIELD

The present invention is related to the medicinal field, in particular, is related to a compound having EZH1/2 inhibitory activity, or a deuterated compound thereof, or a stereoisomer thereof, pharmaceutically acceptable salt, and its use thereof in the preparation of a medicament for used in the treatment of relevant diseases.

### BACKGROUND ART

Cancer treatment methods currently mainly include radiation therapy, surgery therapy, and drug therapy. Drug therapy targeting lesions has become the main means of clinical tumor treatment today. However, due to the rapid emergence of drug resistance in tumor cells, people are basically helpless about tumor metastasis and recurrence at this stage.

Lysine methyltransferase can methylate histones and non-histones, and its abnormal expression is closely related to the occurrence of various tumors. It has become a hot spot in the field of epigenetics for more than ten years. Targeting lysine methyltransferases to reverse abnormal histone or non-histone methylation levels is regarded as another new approach for cancer treatment. PRC2 (polycomb repressive complex 2) is a multi-subunit protein complex composed of EZH1 (Enhancer of zeste homologue 1, KMT6B) or EZH2 (Enhancer of zeste homologue 2, KMT6A), SUZ12 (Suppressor of zeste 12), EED (Embyronic ectoderm development), used to catalyze H3K27 trimethylation. The PCR2 complex methylates the nucleosomal protein H3K9 and lysine 27 through the SET domain of EZH2, and then triggers the aggregation of the PCR1 complex at specific gene sites to silence target genes (CDKN1C, CDH1, RUNX3, etc.), promote cell proliferation. Studies have shown that EZH2 overexpression or SET region mutation (Y641F, Y641N, A687V, A677G point mutation) can all lead to abnormal increase of H3K27me3, promote the growth and development of various types of tumors, such as breast cancer, prostate cancer, leukemia and the like.

However, the activity of EZH1/2 inhibitors in the prior art is still not good enough, and a compound with better activity is urgently needed.

### SUMMARY OF THE INVENTION

The purpose of the present invention is to provide a new small molecule inhibitor targeting EZH1/2.

In the first embodiment of the present invention, the present invention provides a compound represented by Formula I or Formula II, or a deuterated compound thereof, or a stereoisomer thereof, or a pharmaceutically acceptable salt thereof:

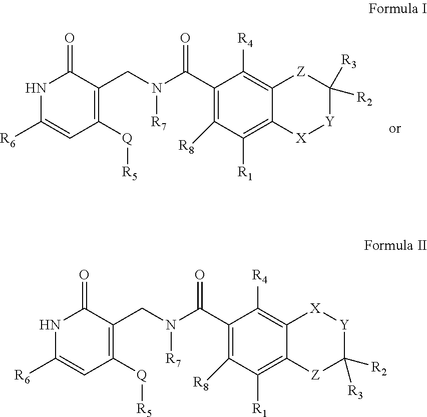

wherein,
X is selected from a group consisting of single bond, O, NR^{X1} or CHR^{X2};
Y is selected from a group consisting of O, S, NR^{Y1} or CHR²;
Z is selected from a group consisting of C=O, O, S, NR^{Z1} or CHR^{Z2};
Q is selected from a group consisting of single bond, O or S;
when X is single bond, Y and Z are not both O;
R^{X1} is selected from a group consisting of the following groups: hydrogen, —C_{1˜6}alkyl, halo-C_{1˜6}alkyl, —C_{0˜2}alkylene-(3˜10 membered cycloalkyl), —C_{0˜2}alkylene-(3˜10 membered heterocycloalkyl), —C_{0˜2}alkylene-(5˜10 membered aryl ring), —C_{0˜2}alkylene-(5˜10 membered heteroaryl ring);
R^{X2} is selected from a group consisting of the following groups: hydrogen, halogen, cyano, hydroxyl, —C_{1˜6}alkyl, halo-C_{1˜6}alkyl, —O(C_{1˜6}alkyl), —O(halo-C_{1˜6}alkyl), —NH₂, —NH(C_{1˜6}alkyl), —N(C_{1˜6}alkyl) (C_{1˜6}alkyl), —C_{0˜2}alkylene-(3˜10 membered cycloalkyl), —C_{0˜2}alkylene-(3˜10 membered heterocycloalkyl), —C_{0˜2}alkylene-(5-10 membered aryl ring), —C_{0˜2}alkylene-(5-10 membered heteroaryl ring);
R^{Y1} is selected from a group consisting of the following groups: hydrogen, —C_{1˜6}alkyl, halo-C_{1˜6}alkyl, —C_{0˜2}alkylene-(3˜10 membered cycloalkyl), —C_{0˜2}alkylene-(3˜10 membered heterocycloalkyl), —C_{0˜2}alkylene-(5˜10 membered aryl ring), —C_{0˜2}alkylene-(5˜10 membered heteroaryl ring);
R^{Y2} is selected from a group consisting of the following groups: hydrogen, halogen, cyano, hydroxyl, —C_{1˜6}alkyl, halo-C_{1˜6}alkyl, —O(C_{1˜6}alkyl), —O(halo-C_{1˜6}alkyl), —NH₂, —NH(C_{1˜6}alkyl), —N(C_{1˜6}alkyl) (C_{1˜6}alkyl), —C_{0˜2}alkylene-(3˜10 membered cycloalkyl), —C_{0˜2}alkylene-(3˜10 membered heterocycloalkyl), —C_{0˜2}alkylene-(5-10 membered aryl ring), —C_{0˜2}alkylene-(5-10 membered heteroaryl ring);
R^{Z1} is selected from a group consisting of the following groups: hydrogen, C_{1˜6}alkyl, halo-C_{1˜6}alkyl, —C_{0˜2}alkylene-(3˜10 membered cycloalkyl), —C_{0˜2}alkylene-(3˜10 membered heterocycloalkyl), —C_{0˜2}alkylene-(5˜10 membered aryl ring), —C_{0˜2}alkylene-(5˜10 membered heteroaryl ring);
R^{Z2} is selected from a group consisting of the following groups: hydrogen, halogen, cyano, hydroxyl, —C_{1˜6}alkyl, halo-C_{1˜6}alkyl, —O(C_{1˜6}alkyl), —O(halo-C_{1˜6}alkyl), —NH₂, —NH(C_{1˜6}alkyl), —N(C_{1˜6}alkyl) (C_{1˜6}alkyl), —C_{0˜2}alkylene-(3˜10 membered cycloalkyl), —C_{0˜2}alkylene-(3˜10 membered heterocycloalkyl), —C_{0˜2}alkylene-(5-10 membered aryl ring), —C_{0˜2}alkylene-(5-10 membered heteroaryl ring);
R₁ is selected from a group consisting of the following groups: hydrogen, halogen, cyano, nitro, amino, hydroxyl, —C_{1˜6}alkyl, —O(C_{1˜6}alkyl), halo-C_{1˜6}alkyl, —C_{0˜2}alkylene-OR^{1b}, —C_{0˜2}alkylene-C(O)R^{1b}, —C_{0˜2}alkylene-C(O)NR^{1b}R^{1c}, —C_{0˜2}alkylene-NR^{1b}R^{1c}, —C_{0˜2}alkylene-NR^{1b}C(O)R^{1c}, —C_{0˜4}alkylene-S(O)₂R^{1b}, —C_{0˜2}alkylene-(3˜10 membered cycloalkyl), —C_{0˜2}alkylene-(3˜10 membered heterocycloalkyl), —C_{0˜2}alkylene-(5˜10 membered aryl ring), —C_{0˜2}alkylene-(5-10 membered heteroaryl ring); wherein said alkyl, alkylene, cycloalkyl, heterocycloalkyl, aryl ring, heteroaryl ring is optionally further substituted by one, two or three independent R^{1b};
R^{1b}, R^{1c} is each independently selected from a group consisting of the following groups: hydrogen, —OH, C_{1˜6}alkyl, halo-C_{1˜6}alkyl, —O(C_{1˜6}alkyl), —O(halo-C_{1˜6}alkyl), —NH₂, —NH(C_{1˜6}alkyl), —N(C_{1˜6}alkyl) (C_{1˜6}alkyl), —C_{0˜2}alkylene-(3˜10 membered cycloalkyl), —C_{0˜2}alkylene-(3˜10 membered heterocycloalkyl), —C_{0˜2}alkylene-(5-10 membered aryl ring), —C_{0˜2}alkylene-(5-10 membered heteroaryl ring);
R₂ is selected from a group consisting of the following groups: —C_{0˜2}alkylene-(3-10 membered cycloalkyl), —C_{0˜2}alkylene-(3˜10 membered heterocycloalkyl); wherein said alkylene, cycloalkyl, heterocycloalkyl is optionally further substituted by one, two or three independent R^{2b};
R^{2b} is each independently selected from a group consisting of the following groups: hydrogen, halogen, cyano, nitro, amino, hydroxyl, —C_{1˜6}alkyl, —O(C_{1˜6}alkyl), halo-C_{1˜6}alkyl, —C_{0˜2}alkylene-OR^{2c}, —C_{0˜2}alkylene-C(O)R^{2c}, —C_{0˜2}alkylene-C(O)NR^{2c}R^{2d}, —C_{0˜2}alkylene-NR^{2c}R^{2d}, —C_{0˜2}alkylene-NR^{2c}C(O)R^{2d}, —C_{0˜4}alkylene-S(O)₂R^{2c}, —C_{0˜2}alkylene-(3˜10 membered cycloalkyl), —C_{0˜2}alkylene-(3˜10 membered heterocycloalkyl), —C_{0˜2}alkylene-(5˜10 membered aryl ring), —C_{0˜2}alkylene-(5-10 membered heteroaryl ring); wherein alkyl, alkylene, cycloalkyl, heterocycloalkyl, aryl ring, heteroaryl ring is optionally further substituted by one, two or three independent R^{2c};
R^{2c}, R^{2d} is each independently selected from a group consisting of hydrogen, cyano, nitro, —OH, halogen, —C_{1˜6}alkyl, halo-C_{1˜6}alkyl, —C_{0˜2}alkylene-O(C_{1˜6}alkyl), —O(halo-C_{1˜6}alkyl), —O(3˜10 membered cycloalkyl), —O(3˜10 membered heterocycloalkyl), —NH₂, —NH(C_{1˜6}alkyl), —N(C₁˜6alkyl) (C_{1˜6}alkyl), —C_{0˜2}alkylene-(3˜10 membered cycloalkyl), —C_{0˜2}alkylene-(3˜10 membered heterocycloalkyl), —C_{0˜2}alkylene-(5˜10 membered aryl ring), —C_{0˜2}alkylene-(5˜10 membered heteroaryl ring); wherein said alkyl, alkylene, cycloalkyl, heterocycloalkyl, aryl ring, heteroaryl ring is optionally further substituted by one, two or three independent R^{2e};
R^{2e} is each independently selected from a group consisting of hydrogen, cyano, nitro, —OH, halogen, —C_{1˜6}alkyl, halo-C_{1˜6}alkyl, —C_{0˜2}alkylene-O(C_{1˜6}alkyl), —O(halo-C_{1˜6}alkyl), —O(3˜10 membered cycloalkyl), —O(3˜10 membered heterocycloalkyl), —NH₂, —NH(C_{1˜6}alkyl), —N(C_{1˜6}alkyl) (C_{1˜6}alkyl), —C_{0˜2}alkylene-(3˜10 membered cycloalkyl), —C_{0˜2}alkylene-(3˜10 membered heterocycloalkyl), —C_{0˜2}alkylene-(5-10 membered aryl ring), —C_{0˜2}alkylene-(5-10 membered heteroaryl ring);
R₃ is selected from a group consisting of the following groups: hydrogen, halogen, cyano, nitro, amino, —OH, —C_{1˜6}alkyl, —O(C_{1˜6}alkyl), halo-C_{1˜6}alkyl, —C_{0˜2}alkylene-OR^{3b}, —C_{0˜2}alkylene-C(O)R^{3b}, —C_{0˜2}alkylene-C(O)NR^{3b}R^{3c}, —C_{0˜2}alkylene-NR^{3b}R^{3c}, —C_{0˜2}alkylene-NR^{3b}C(O)R^{3c}, —C_{0˜4}alkylene-S(O)₂R^{3b}, —C_{0˜2}alkylene-(3˜10 membered cycloalkyl), —C_{0˜2}alkylene-(3˜10 membered heterocycloalkyl);
R^{3b}, R^{3c} is each independently selected from a group consisting of the following groups: hydrogen, halogen, cyano, nitro, —OH, C_{1˜6}alkyl, halo-C_{1˜6}alkyl, —O(C_{1˜6}alkyl), —O(halo-C_{1˜6}alkyl), —NH₂, —NH(C_{1˜6}alkyl), —N(C_{1˜6}alkyl) (C_{1˜6}alkyl), —C_{0˜2}alkylene-(3˜10 membered cycloalkyl), —C_{0˜2}alkylene-(3˜10 membered heterocycloalkyl), —C_{0˜2}alkylene-(5˜10 membered aryl ring), —C_{0˜2}alkylene-(5˜10 membered heteroaryl ring);
R₄ is selected from a group consisting of the following groups: hydrogen, halogen, cyano, nitro, hydroxyl, —C_{1˜6}alkyl, halo-C_{1˜6}alkyl, —O(C_{1˜6}alkyl), —O(halo-C_{1˜6}alkyl), —NH₂, —NH(C_{1˜6}alkyl), —N(C_{1˜6}alkyl) (C_{1˜6}alkyl), —C_{0˜2}alkylene-(3˜10 membered cycloalkyl), —C_{0˜2}alkylene-(3˜10 membered heterocycloalkyl), —C_{0˜2}alkylene-(5-10 membered aryl ring), —C_{0˜2}alkylene-(5-10 membered heteroaryl ring); wherein said alkyl, alkylene, cycloalkyl, heterocycloalkyl, aryl ring, heteroaryl ring is optionally further substituted by one, two or three independent R^{4b};
R^{4b} is each independently selected from a group consisting of the following groups: hydrogen, halogen, cyano, nitro, —OH, C_{1˜6}alkyl, halo-C_{1˜6}alkyl, —O(C_{1˜6}alkyl), —O(halo-C_{1˜6}alkyl), —NH₂, —NH(C_{1˜6}alkyl), —N(C_{1˜6}alkyl) (C_{1˜6}alkyl), —C_{0˜2}alkylene-(3˜10 membered cycloalkyl), —C_{0˜2}alkylene-(3˜10 membered heterocycloalkyl), —C_{0˜2}alkylene-(5˜10 membered aryl ring), —C_{0˜2}alkylene-(5˜10 membered heteroaryl ring);
R₈ is selected from a group consisting of the following groups: hydrogen, halogen, cyano, nitro, hydroxyl, —C_{1˜6}alkyl, halo-C_{1˜6}alkyl, —O(C_{1˜6}alkyl), —O(halo-C_{1˜6}alkyl), —NH₂, —NH(C_{1˜6}alkyl), —N(C_{1˜6}alkyl) (C_{1˜6}alkyl), —C_{0˜2}alkylene-(3˜10 membered cycloalkyl), —C_{0˜2}alkylene-(3˜10 membered heterocycloalkyl), —C_{0˜2}alkylene-(5-10 membered aryl ring), —C_{0˜2}alkylene-(5-10 membered heteroaryl ring);
R₆ is selected from a group consisting of the following groups: hydrogen, halogen, cyano, nitro, hydroxyl, —C_{1˜6}alkyl, halo-C_{1˜6}alkyl, —O(C_{1˜6}alkyl), —O(halo-C_{1˜6}alkyl), —NH₂, —NH(C_{1˜6}alkyl), —N(C_{1˜6}alkyl) (C_{1˜6}alkyl), —C_{0˜2}alkylene-(3˜10 membered cycloalkyl), —C_{0˜2}alkylene-(3˜10 membered heterocycloalkyl), —C_{0˜2}alkylene-(5-10 membered aryl ring), —C_{0˜2}alkylene-(5-10 membered heteroaryl ring);
R₇ is selected from a group consisting of the following groups: hydrogen, —C_{1˜6}alkyl, halo-C_{1˜6}alkyl, —C_{0˜2}alkylene-(3˜10 membered cycloalkyl), —C_{0˜2}alkylene-(3˜10 membered heterocycloalkyl), —C_{0˜2}alkylene-(5˜10 membered aryl ring), —C_{0˜2}alkylene-(5˜10 membered heteroaryl ring);
R₈ is selected from a group consisting of the following groups: hydrogen, halogen, cyano, nitro, —OH, C_{1˜6}alkyl, halo-C_{1˜6}alkyl, —O(C_{1˜6}alkyl), —O(halo-C_{1˜6}alkyl), —NH₂, —NH(C_{1˜6}alkyl), —N(C_{1˜6}alkyl) (C_{1˜6}alkyl), —C_{0˜2}alkylene-(3˜10 membered cycloalkyl), —C_{0˜2}alkylene-(3˜10 membered heterocycloalkyl), —C_{0˜2}alkylene-(5-10 membered aryl ring), —C_{0˜2}alkylene-(5-10 membered heteroaryl ring);
or, R⁷, R⁸ and the atom directly bound thereto form a 4-10 membered heterocyclic ring; wherein said heterocyclic ring is optionally further substituted by one, two or three independent R⁷¹; hydrogen, halogen, cyano, nitro, —OH, C_{1˜6}alkyl, halo-C_{1˜6}alkyl, —O(C_{1˜6}alkyl), —O(halo-C_{1˜6}alkyl), —NH₂, —NH(C_{1˜6}alkyl), —N(C₁₋₆alkyl) (C_{1˜6}alkyl), —C_{0˜2}alkylene-(3˜10 membered cycloalkyl), —C_{0˜2}alkylene-(3˜10 membered heterocycloalkyl), —C_{0˜2}alkylene-(5-10 membered aryl ring), —C_{0˜2}alkylene-(5-10 membered heteroaryl ring).

In the first embodiment of the present invention, it provides the compound of the present invention, or a deuterated compound thereof, or a stereoisomer thereof, or a pharmaceutically acceptable salt thereof, wherein said R₁, R₃, R₄, R₅, R₆, R₇, R₈ is each independently selected from a group consisting of hydrogen, halogen, —C_{1˜3}alkyl, halo-C_{1˜3}alkyl.

In the first embodiment of the present invention, it provides the compound of the present invention, or a deuterated compound thereof, or a stereoisomer thereof, or a pharmaceutically acceptable salt thereof, wherein said R₁, R₃, R₄, R₅, R₆, R₇, R₈ is each independently selected from a group consisting of hydrogen, Cl, methyl, ethyl, —CF₃.

In the first embodiment of the present invention, it provides the compound of the present invention, or a deuterated compound thereof, or a stereoisomer thereof, or a pharmaceutically acceptable salt thereof, wherein said R₂ is selected from a group consisting of -(3˜10 membered cycloalkyl), -(3˜10 membered heterocycloalkyl); wherein said cycloalkyl, heterocycloalkyl is optionally further substituted by one, two or three independent R^{2b};
R^{2b} is selected from a group consisting of hydrogen, halogen, —C₁₋₆alkyl, —NR^{2c}R^{2d}, —NR^{2c}C(O)R^{2d}, —C_{0˜2}alkylene-(3˜10 membered cycloalkyl), —C_{0˜2}alkylene-(3˜10 membered heterocycloalkyl); wherein said alkyl, cycloalkyl, heterocycloalkyl is optionally further substituted by one, two or three independent R^{2c};
R^{2c}, R^{2d} is each independently selected from a group consisting of hydrogen, halogen, —C_{1˜6}alkyl, halo-C_{1˜6}alkyl, —O(C_{1˜6}alkyl), —O(halo-C_{1˜6}alkyl), —O(3˜10 membered cycloalkyl), -(3˜10 membered cycloalkyl), -(3˜10 membered heterocycloalkyl); wherein said alkyl, cycloalkyl, heterocycloalkyl is optionally further substituted by one, two or three independent R^{2e};
R^{2e} is each independently selected from a group consisting of hydrogen, halogen, —O(C_{1˜6}alkyl).

In the first embodiment of the present invention, it provides the compound of the present invention, or a deuterated compound thereof, or a stereoisomer thereof, or a pharmaceutically acceptable salt thereof, wherein said R₂ is selected from a group consisting of

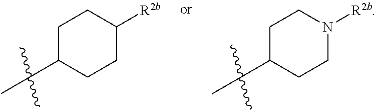

In the first embodiment of the present invention, it provides the compound of the present invention, or a deuterated compound thereof, or a stereoisomer thereof, or a pharmaceutically acceptable salt thereof, wherein said R₂ is specifically selected from a group consisting of

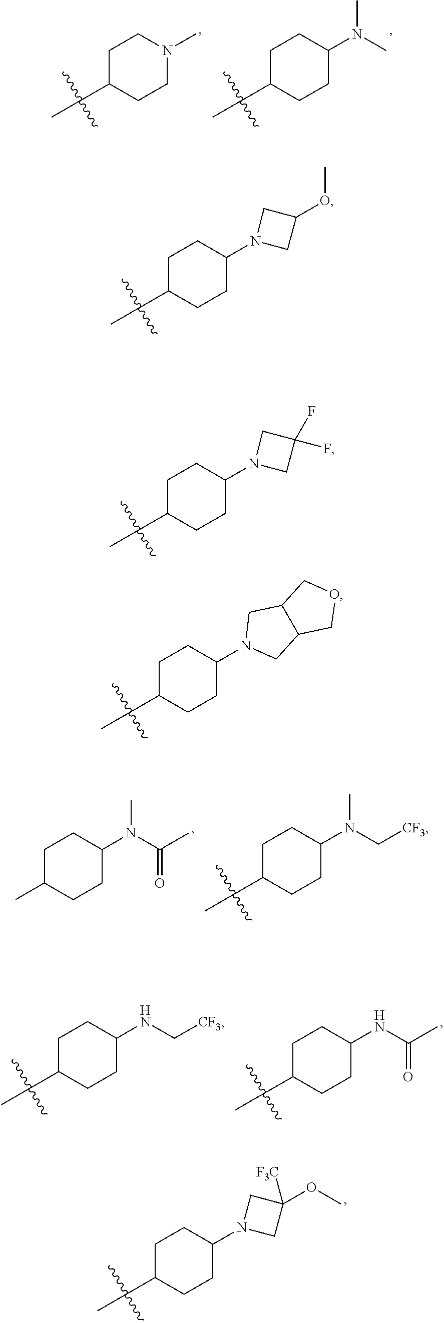

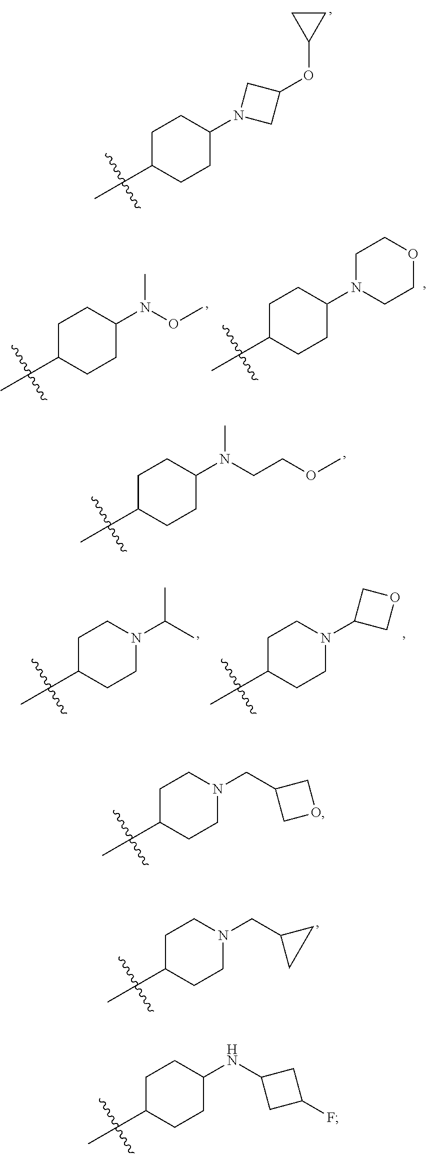

preferably, said R₂ is specifically selected from a group consisting of

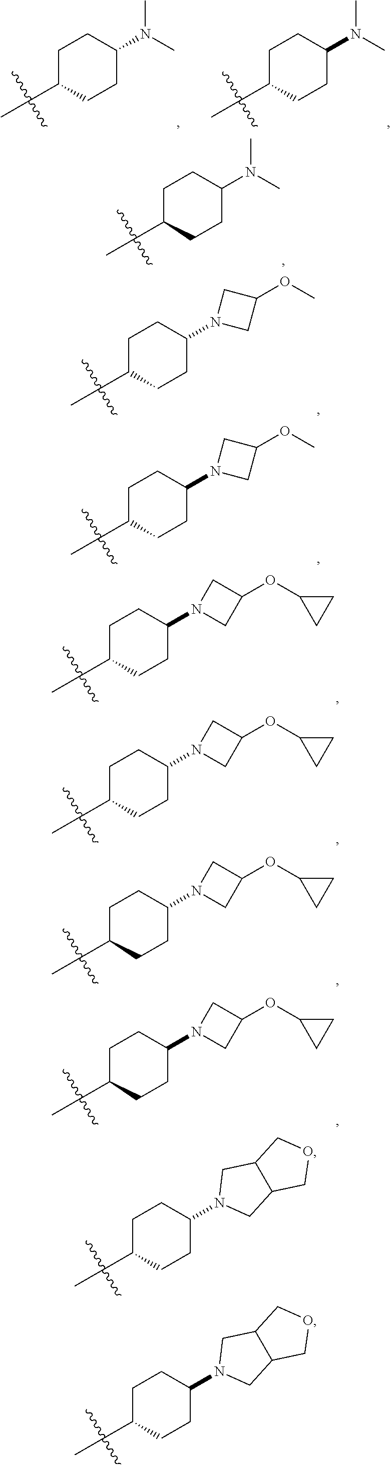

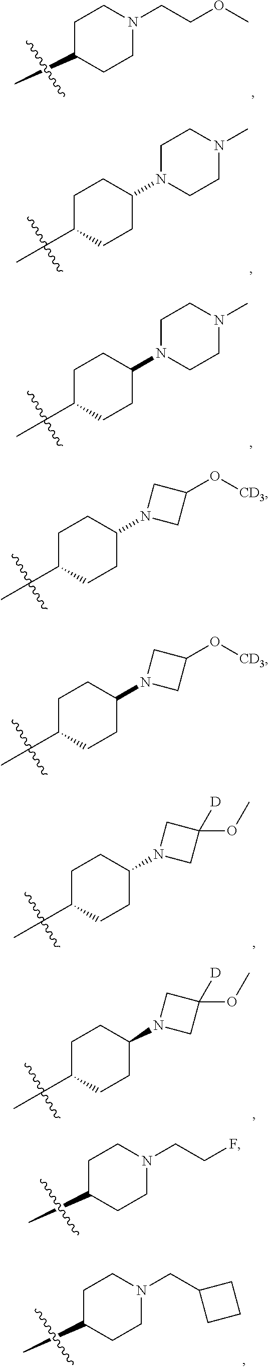

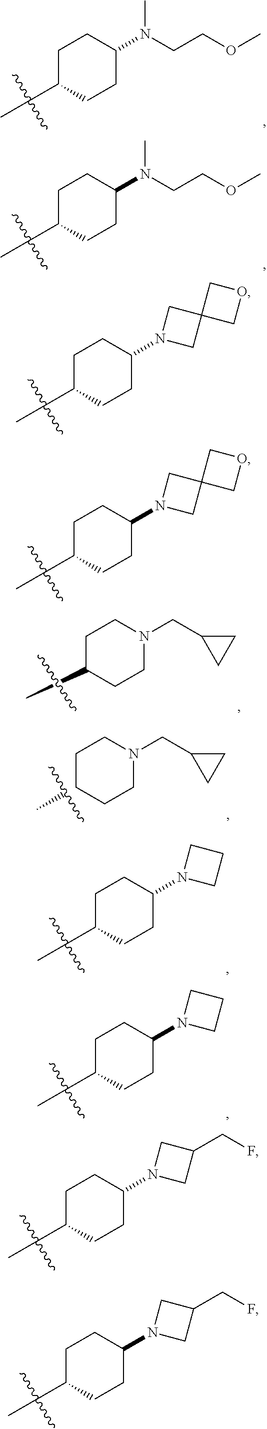

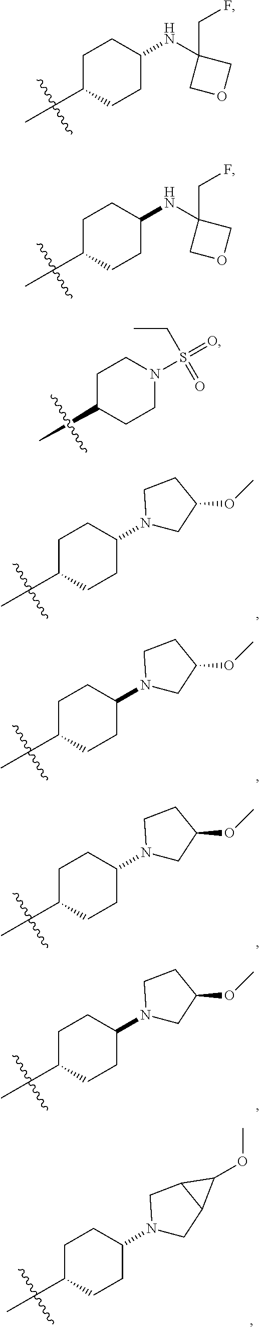

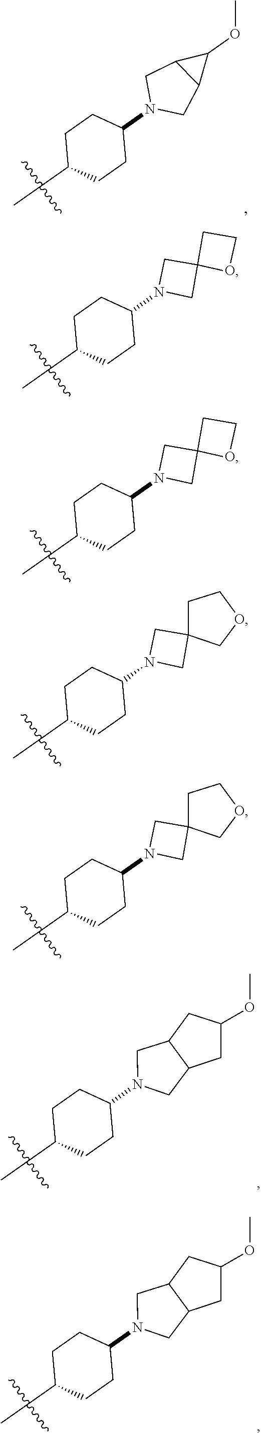

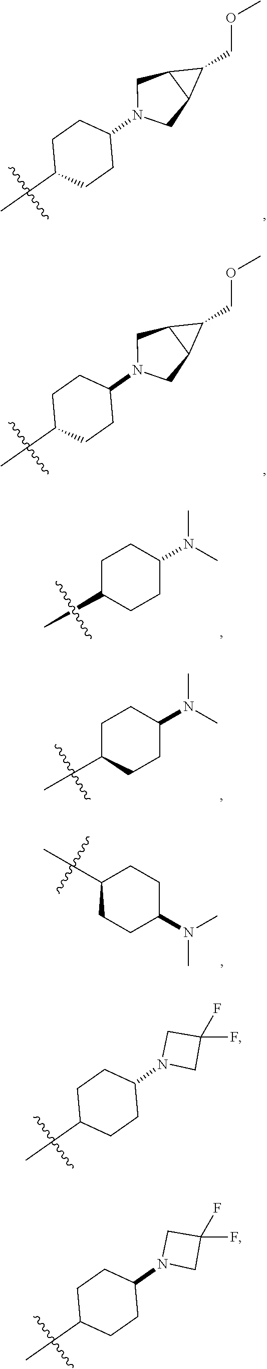

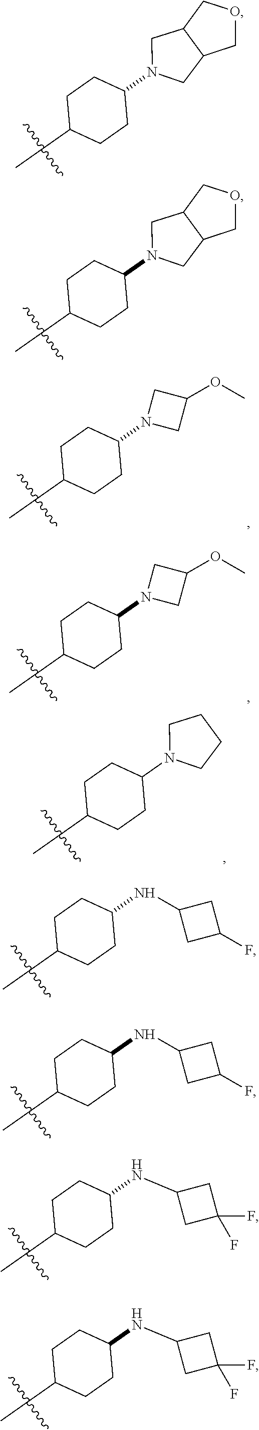

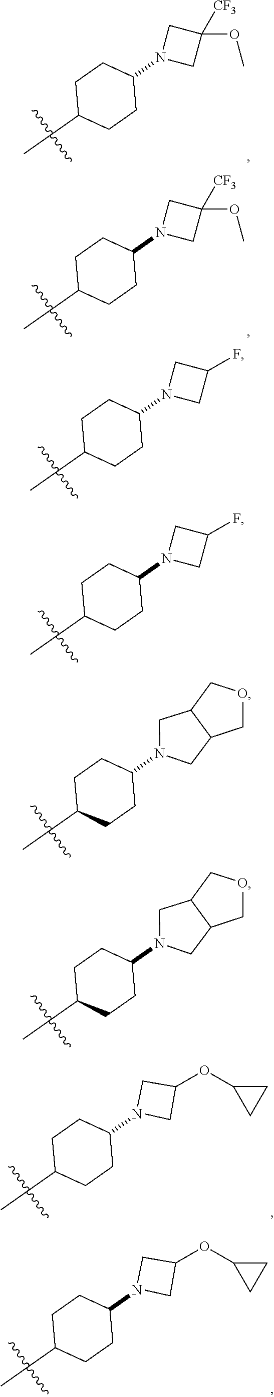

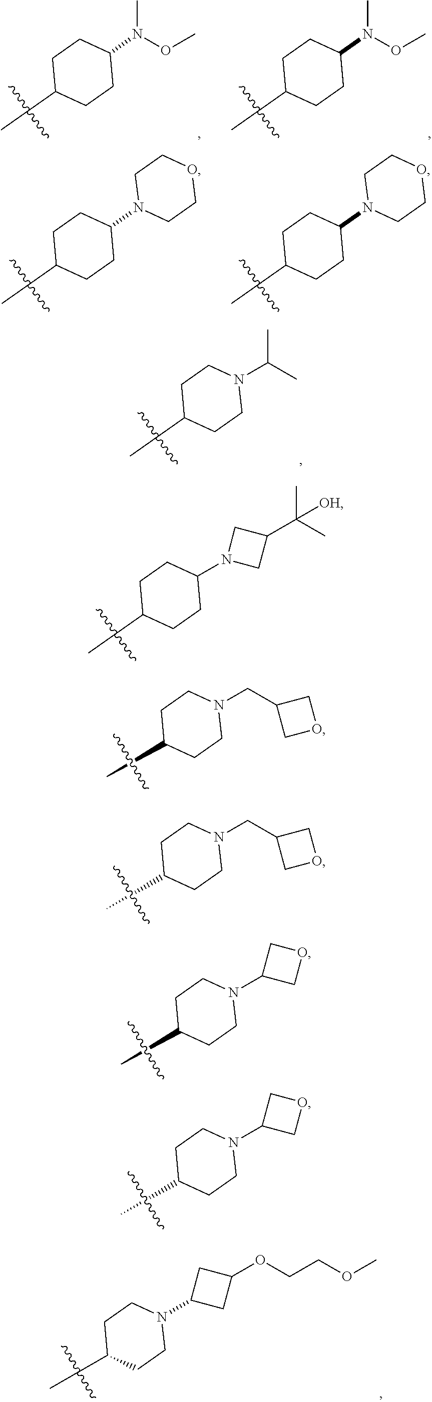

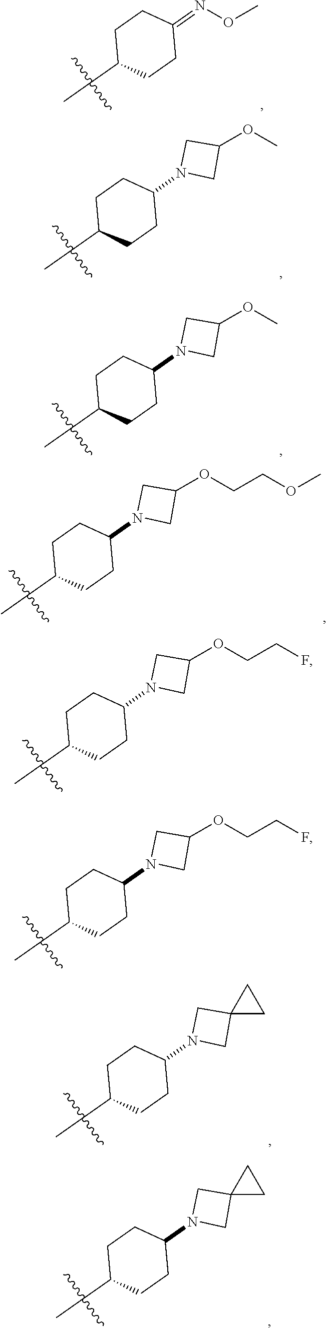

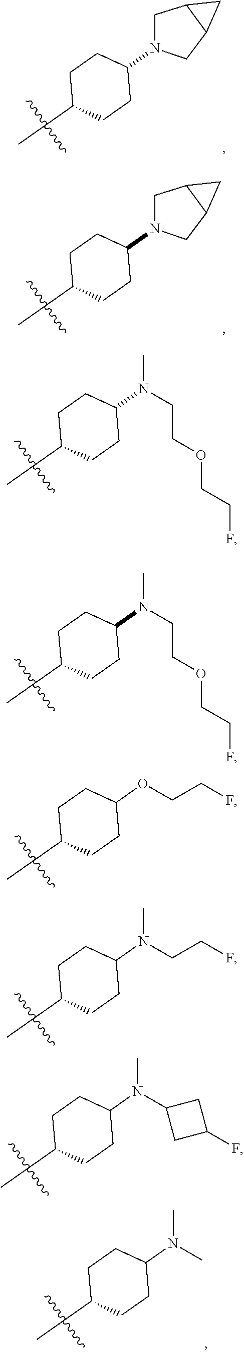

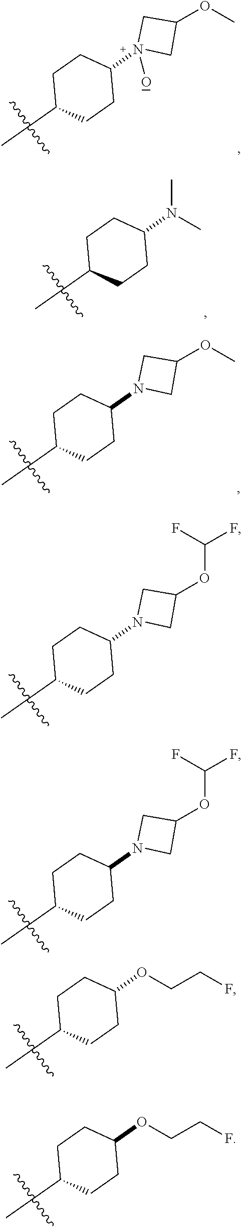

In the first embodiment of the present invention, it provides the compound of the present invention, or a deuterated compound thereof, or a stereoisomer thereof, or a pharmaceutically acceptable salt thereof, wherein, X is selected from a group consisting of single bond, O, CHR^{X2}; y is selected from a group consisting of O, NR^{Y1} or CHR²; Z is selected from a group consisting of —C=O, O, NR^{Z1} or CHR^{Z2}; Q is selected from a group consisting of single bond, O or S;
R^{X2} is selected from a group consisting of hydrogen, —C_{1˜3}alkyl;
R^{Y1} is selected from a group consisting of hydrogen, —C_{1˜3}alkyl, halo-C_{1˜3}alkyl;
R^{Y2} is selected from a group consisting of hydrogen, —C_{1˜3}alkyl, halo-C_{1˜3}alkyl;
R^{Z1} is selected from a group consisting of hydrogen, —C_{1˜3}alkyl, -(3-6 membered cycloalkyl), halo-C_{1˜3}alkyl;
R^{Z2} is selected from a group consisting of hydrogen, —C_{1˜3}alkyl, halo-C_{1˜3}alkyl.

In the first embodiment of the present invention, it provides the compound of the present invention, or a deuterated compound thereof, or a stereoisomer thereof, or a pharmaceutically acceptable salt thereof, wherein said R^{Y1} is selected from a group consisting of methyl; R^{Y2} is selected from a group consisting of hydrogen, methyl; R^{Z1} is selected from a group consisting of hydrogen, methyl, ethyl, cyclopropyl.

In the second embodiment of the present invention, it provides the compound of the present invention, or a deuterated compound thereof, or a stereoisomer thereof, or a pharmaceutically acceptable salt thereof, including the following compounds represented by Formula IIa:

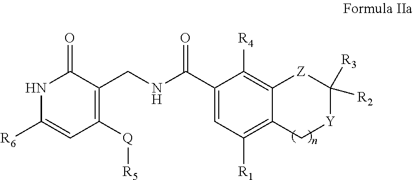

Q is selected from a group consisting of single bond, O or S;
   Z is selected from a group consisting of C═O, O, NR^{Z1} or CR^{Z2};
   Y is selected from a group consisting of O, NR^{Y1} or CHR¹²; n is 0 or 1;
   when n is 0, Y and Z are not both O;
R^{Y1} is selected from a group consisting of hydrogen, —C_{1˜6}alkyl, halo-C_{1˜6}alkyl, —C_{0˜2}alkylene-(3˜10 membered cycloalkyl), —C_{0˜2}alkylene-(3˜10 membered heterocycloalkyl), —C_{0˜2}alkylene-(5-10 membered aryl ring), —C_{0˜2}alkylene-(5-10 membered heteroaryl ring);
R^{Y2} is selected from a group consisting of hydrogen, halogen, cyano, hydroxyl, —C_{1˜6}alkyl, halo-C_{1˜6}alkyl, —O(C_{1˜6}alkyl), —O(halo-C_{1˜6}alkyl), —NH₂, —NH(C_{1˜6}alkyl), —N(C_{1˜6}alkyl) (C_{1˜6}alkyl), —C_{0˜2}alkylene-(3˜10 membered cycloalkyl), —C_{0˜2}alkylene-(3˜10 membered heterocycloalkyl), —C_{0˜2}alkylene-(5-10 membered aryl ring), —C_{0˜2}alkylene-(5-10 membered heteroaryl ring);
R^{Z1} is selected from a group consisting of hydrogen, —C_{1˜6}alkyl, halo-C_{1˜6}alkyl, —C_{0˜2}alkylene-(3˜10 membered cycloalkyl), —C_{0˜2}alkylene-(3˜10 membered heterocycloalkyl), —C_{0˜2}alkylene-(5-10 membered aryl ring), —C_{0˜2}alkylene-(5-10 membered heteroaryl ring);
R^{Z2} is selected from a group consisting of hydrogen, halogen, cyano, hydroxyl, —C_{1˜6}alkyl, halo-C_{1˜6}alkyl, —O(C_{1˜6}alkyl), —O(halo-C_{1˜6}alkyl), —NH₂, —NH(C_{1˜6}alkyl), —N(C_{1˜6}alkyl) (C_{1˜6}alkyl), —C_{0˜2}alkylene-(3˜10 membered cycloalkyl), —C_{0˜2}alkylene-(3˜10 membered heterocycloalkyl), —C_{0˜2}alkylene-(5-10 membered aryl ring), —C_{0˜2}alkylene-(5-10 membered heteroaryl ring);
R₁ is selected from a group consisting of hydrogen, halogen, cyano, nitro, amino, hydroxyl, —C_{1˜6}alkyl, halo-C_{1˜6}alkyl, —C_{0˜2}alkylene-OR^{1b}, —C_{0˜2}alkylene-C(O)Rb, —C_{0˜2}alkylene-C(O)NR^{1b}R^{1c}, —C_{0˜2}alkylene-NR^{1b}R^{1c}, —C_{0˜2}alkylene-NR^{1b}C(O)R^{1c}, —C_{0˜4}alkylene-S(O)₂R^{1b}, —C_{0˜2}alkylene-(3˜10 membered cycloalkyl), —C_{0˜2}alkylene-(3˜10 membered heterocycloalkyl), —C_{0˜2}alkylene-(5˜10 membered aryl ring), —C_{0˜2}alkylene-(5-10 membered heteroaryl ring); wherein said alkyl, alkylene, cycloalkyl, heterocycloalkyl, aryl ring, heteroaryl ring is optionally further substituted by one, two or three independent R^{1b};
R^{1b}, R^{1c} is each independently selected from a group consisting of hydrogen, —OH, C_{1˜6}alkyl, halo-C_{1˜6}alkyl, —O(C_{1˜6}alkyl), —O(halo-C_{1˜6}alkyl), —NH₂, —NH(C_{1˜6}alkyl), —N(C_{1˜6}alkyl) (C_{1˜6}alkyl), —C_{0˜2}alkylene-(3˜10 membered cycloalkyl), —C_{0˜2}alkylene-(3˜10 membered heterocycloalkyl), —C_{0˜2}alkylene-(5-10 membered aryl ring), —C_{0˜2}alkylene-(5-10 membered heteroaryl ring);
R₂ is selected from a group consisting of —C_{0˜2}alkylene-(3-10 membered cycloalkyl), —C_{0˜2}alkylene-(3˜10 membered heterocycloalkyl); wherein said alkylene, cycloalkyl, heterocycloalkyl is optionally further substituted by one, two or three independent R^{2b};
R^{2b} is each independently selected from a group consisting of hydrogen, halogen, cyano, nitro, amino, hydroxyl, —C_{1˜6}alkyl, —O(C_{1˜6}alkyl), halo-C_{1˜6}alkyl, —C_{0˜2}alkylene-OR^{2c}, —C_{0˜2}alkylene-C(O)R^{2c}, —C_{0˜2}alkylene-C(O)NR^{2c}R^{2d}, —C_{0˜2}alkylene-NR^{2c}R^{2d}, —C_{0˜2}alkylene-NR^{2c}C(O)R^{2d}, —C_{0˜4}alkylene-S(O)₂R^{2c}, —C_{0˜2}alkylene-(3˜10 membered cycloalkyl), —C_{0˜2}alkylene-(3˜10 membered heterocycloalkyl), —C_{0˜2}alkylene-(5˜10 membered aryl ring), —C_{0˜2}alkylene-(5-10 membered heteroaryl ring); wherein alkyl, alkylene, cycloalkyl, heterocycloalkyl, aryl ring, heteroaryl ring is optionally further substituted by one, two or three independent R^{2c};
R^{2c}, R^{2d} is each independently selected from a group consisting of hydrogen, cyano, nitro, —OH, halogen, —C_{1˜6}alkyl, halo-C_{1˜6}alkyl, —C_{0˜2}alkylene-O(C_{1˜6}alkyl), —O(halo-C_{1˜6}alkyl), —O(3˜10 membered cycloalkyl), —O(3˜10 membered heterocycloalkyl), —NH₂, —NH(C_{1˜6}alkyl), —N(C_{1˜6}alkyl) (C_{1˜6}alkyl), —C_{0˜2}alkylene-(3˜10 membered cycloalkyl), —C_{0˜2}alkylene-(3˜10 membered heterocycloalkyl), —C_{0˜2}alkylene-(5˜10 membered aryl ring), —C_{0˜2}alkylene-(5˜10 membered heteroaryl ring); wherein said alkyl, alkylene, cycloalkyl, heterocycloalkyl, aryl ring, heteroaryl ring is optionally further substituted by one, two or three independent R^{2e};
R^{2e} is each independently selected from a group consisting of hydrogen, cyano, nitro, —OH, halogen, —C_{1˜6}alkyl, halo-C_{1˜6}alkyl, —C_{0˜2}alkylene-O(C_{1˜6}alkyl), —O(halo-C_{1˜6}alkyl), —O(3˜10 membered cycloalkyl), —O(3˜10 membered heterocycloalkyl), —NH₂, —NH(C_{1˜6}alkyl), —N(C₁˜6alkyl) (C_{1˜6}alkyl), —C_{0˜2}alkylene-(3˜10 membered cycloalkyl), —C_{0˜2}alkylene-(3˜10 membered heterocycloalkyl), —C_{0˜2}alkylene-(5˜10 membered aryl ring), —C_{0˜2}alkylene-(5˜10 membered heteroaryl ring);
R₃ is selected from a group consisting of hydrogen, halogen, cyano, nitro, amino, —OH, —C_{1˜6}alkyl, —O(C_{1˜6}alkyl), halo-C_{1˜6}alkyl, —C_{0˜2}alkylene-OR^{3b}, —C_{0˜2}alkylene-C(O)R^{3b}, —C_{0˜2}alkylene-C(O)NR^{3b}R^{3c}, —C_{0˜2}alkylene-NR^{3b}R^{3c}, —C_{0˜2}alkylene-NR^{3b}C(O)R^{3c}, —C_{0˜4}alkylene-S(O)₂R^{3b}, —C_{0˜2}alkylene-(3˜10 membered cycloalkyl), —C_{0˜2}alkylene-(3˜10 membered heterocycloalkyl);
R^{3b}, R^{3c} is each independently selected from a group consisting of hydrogen, halogen, cyano, nitro, —OH, C_{1˜6}alkyl, halo-C_{1˜6}alkyl, —O(C_{1˜6}alkyl), —O(halo-C_{1˜6}alkyl), —NH₂, —NH(C_{1˜6}alkyl), —N(C_{1˜6}alkyl) (C_{1˜6}alkyl), —C_{0˜2}alkylene-(3˜10 membered cycloalkyl), —C_{0˜2}alkylene-(3˜10 membered heterocycloalkyl), —C_{0˜2}alkylene-(5-10 membered aryl ring), —C_{0˜2}alkylene-(5-10 membered heteroaryl ring);
R₄ is selected from a group consisting of hydrogen, halogen, cyano, nitro, hydroxyl, —C_{1˜6}alkyl, halo-C_{1˜6}alkyl, —O(C_{1˜6}alkyl), —O(halo-C_{1˜6}alkyl), —NH₂, —NH(C_{1˜6}alkyl), —N(C_{1˜6}alkyl) (C_{1˜6}alkyl), —C_{0˜2}alkylene-(3˜10 membered cycloalkyl), —C_{0˜2}alkylene-(3˜10 membered heterocycloalkyl), —C_{0˜2}alkylene-(5˜10 membered aryl ring), —C_{0˜2}alkylene-(5˜10 membered heteroaryl ring); wherein said alkyl, alkylene, cycloalkyl, heterocycloalkyl, aryl ring, heteroaryl ring is optionally further substituted by one, two or three independent R^{4b};
R^{4b} is each independently selected from a group consisting of hydrogen, halogen, cyano, nitro, —OH, C_{1˜6}alkyl, halo-C_{1˜6}alkyl, —O(C_{1˜6}alkyl), —O(halo-C_{1˜6}alkyl), —NH₂, —NH(C_{1˜6}alkyl), —N(C_{1˜6}alkyl) (C_{1˜6}alkyl), —C_{0˜2}alkylene-(3˜10 membered cycloalkyl), —C_{0˜2}alkylene-(3˜10 membered heterocycloalkyl), —C_{0˜2}alkylene-(5-10 membered aryl ring), —C_{0˜2}alkylene-(5-10 membered heteroaryl ring);
R₈ is selected from a group consisting of hydrogen, halogen, cyano, nitro, hydroxyl, —C_{1˜6}alkyl, halo-C_{1˜6}alkyl, —O(C_{1˜6}alkyl), —O(halo-C_{1˜6}alkyl), —NH₂, —NH(C_{1˜6}alkyl), —N(C_{1˜6}alkyl) (C_{1˜6}alkyl), —C_{0˜2}alkylene-(3˜10 membered cycloalkyl), —C_{0˜2}alkylene-(3˜10 membered heterocycloalkyl), —C_{0˜2}alkylene-(5-10 membered aryl ring), —C_{0˜2}alkylene-(5-10 membered heteroaryl ring);
R₆ is selected from a group consisting of hydrogen, halogen, cyano, nitro, hydroxyl, —C_{1˜6}alkyl, halo-C_{1˜6}alkyl, —O(C_{1˜6}alkyl), —O(halo-C_{1˜6}alkyl), —NH₂, —NH(C_{1˜6}alkyl), —N(C_{1˜6}alkyl) (C_{1˜6}alkyl), —C_{0˜2}alkylene-(3˜10 membered cycloalkyl), —C_{0˜2}alkylene-(3˜10 membered heterocycloalkyl), —C_{0˜2}alkylene-(5-10 membered aryl ring), —C_{0˜2}alkylene-(5-10 membered heteroaryl ring).

In the second embodiment of the present invention, it provides the compound of the present invention, or a deuterated compound thereof, or a stereoisomer thereof, or a pharmaceutically acceptable salt thereof, wherein said R₁, R₃, R₄, R₅, R₆ is each independently selected from a group consisting of hydrogen, halogen, —C_{1˜3}alkyl;
Q is selected from a group consisting of single bond, O or S; Z is selected from a group consisting of C═O, O, NR^{Z1} or CR^{Z2}; Y is selected from a group consisting of O or NR^{Y1};
R^{Z1} is selected from a group consisting of hydrogen, —C_{1˜3}alkyl, -(3-6 membered cycloalkyl), halo-C_{1˜3}alkyl;
R^{Z2} is selected from a group consisting of hydrogen, —C_{1˜3}alkyl, halo-C_{1˜3}alkyl;
R^{Y1} is selected from a group consisting of hydrogen, —C_{1˜3}alkyl, halo-C_{1˜3}alkyl.

In the second embodiment of the present invention, it provides the compound of the present invention, or a deuterated compound thereof, or a stereoisomer thereof, or a pharmaceutically acceptable salt thereof, wherein said R₂ is specifically selected from a group consisting of

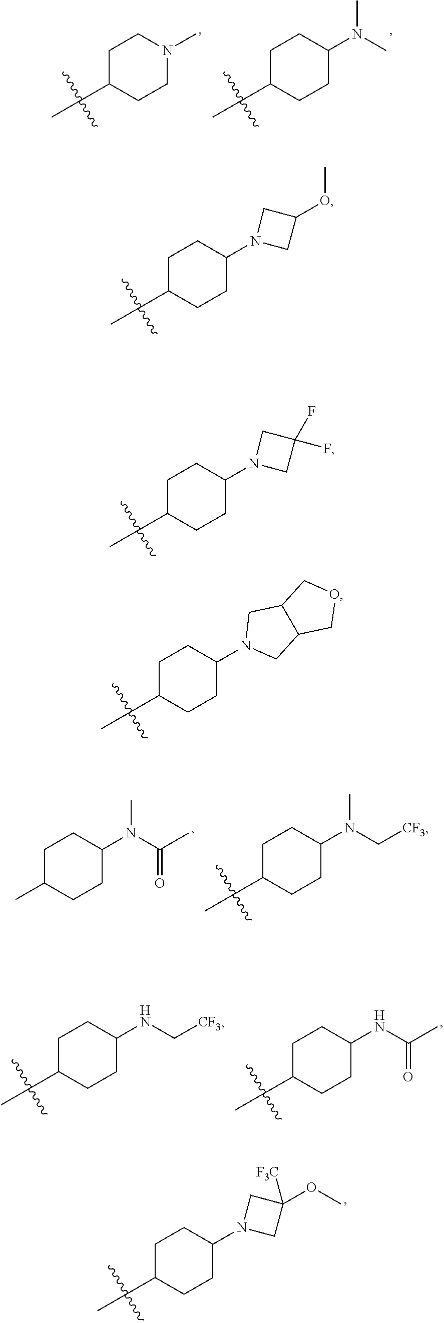

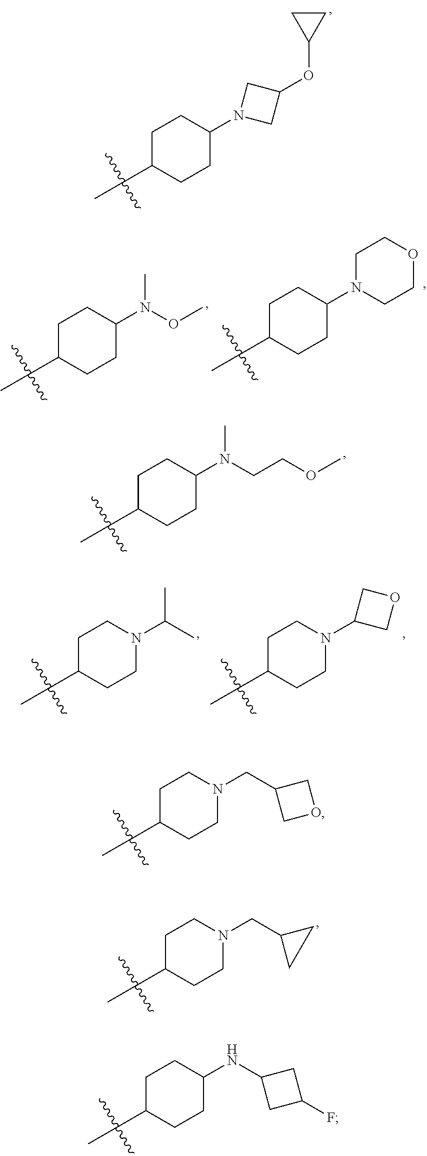

preferably, said R₂ is specifically selected from a group consisting of

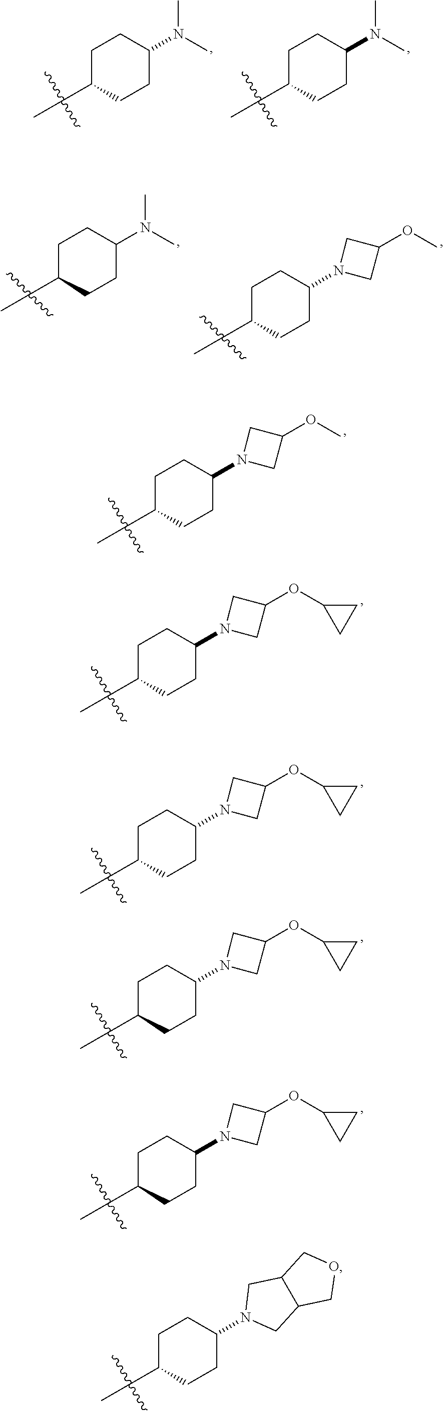

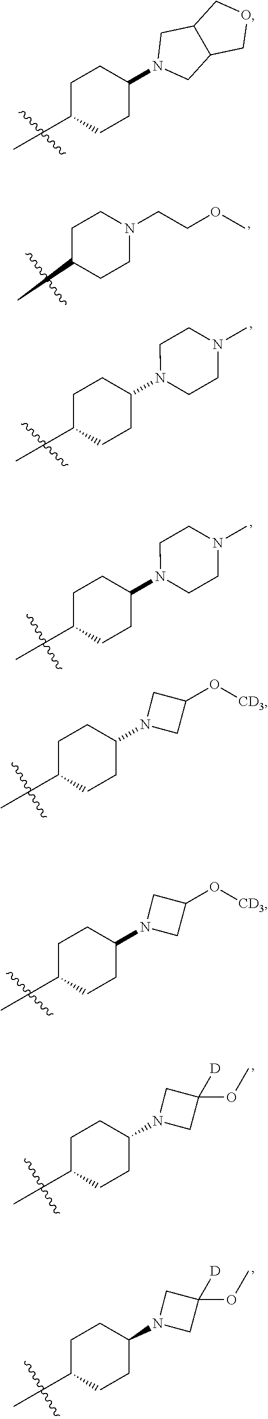

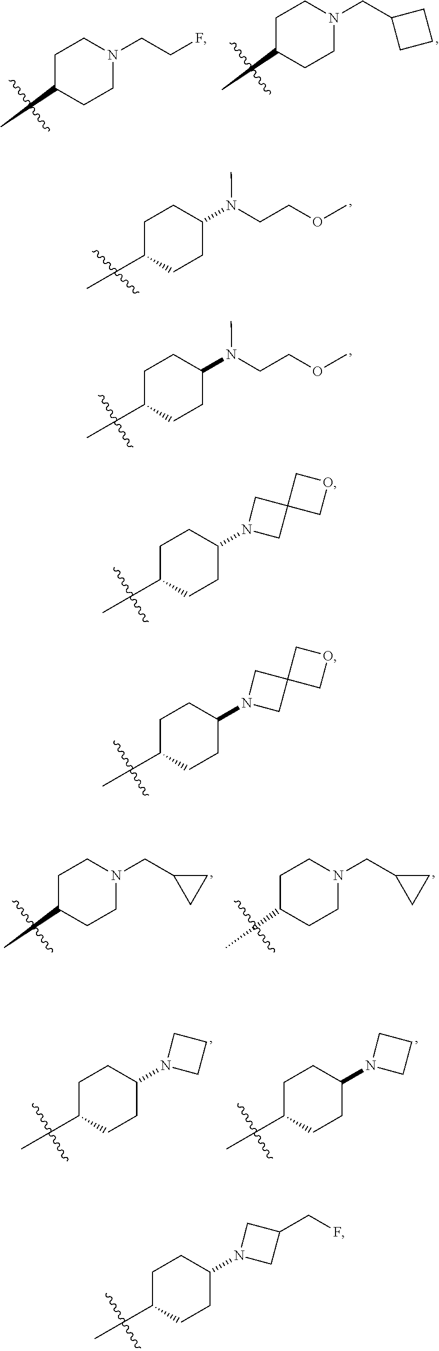

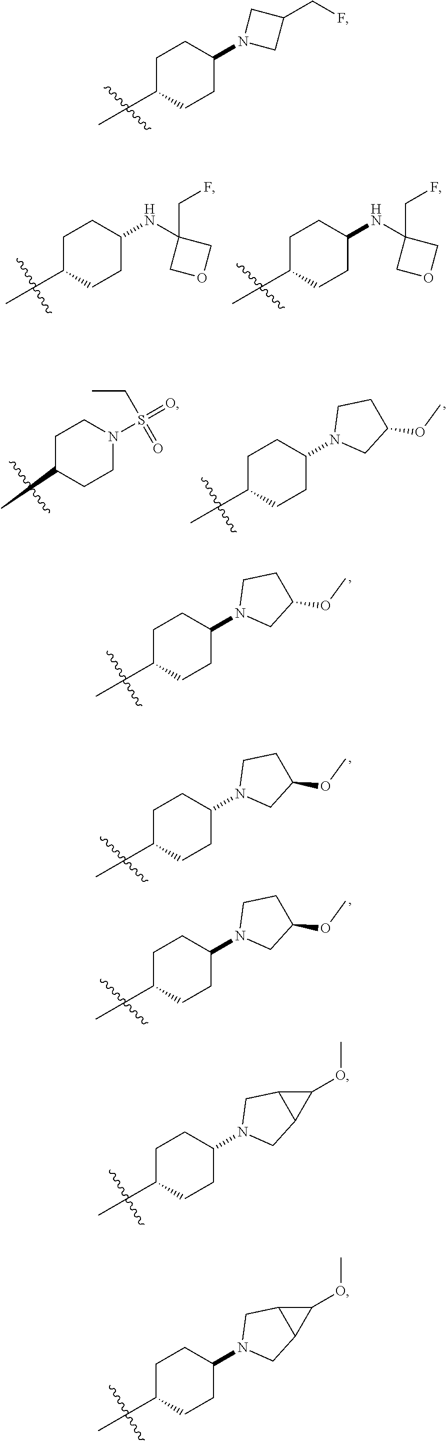

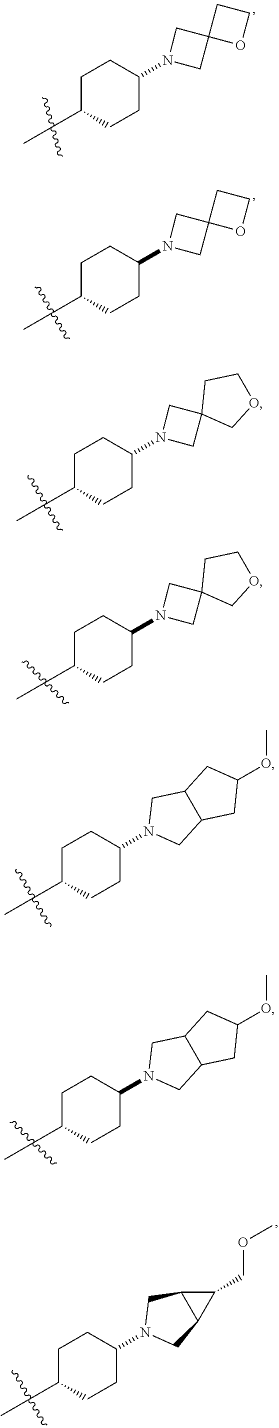

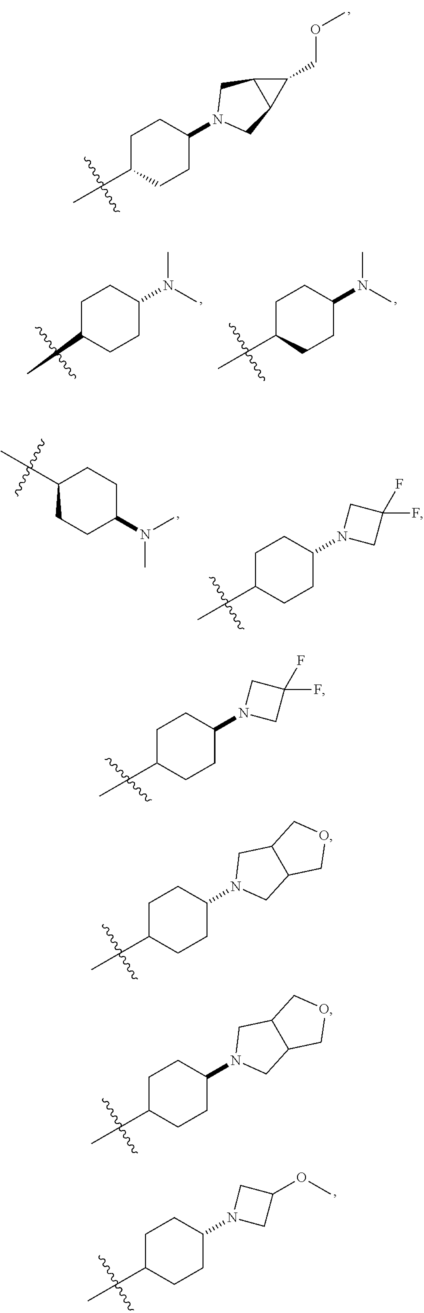

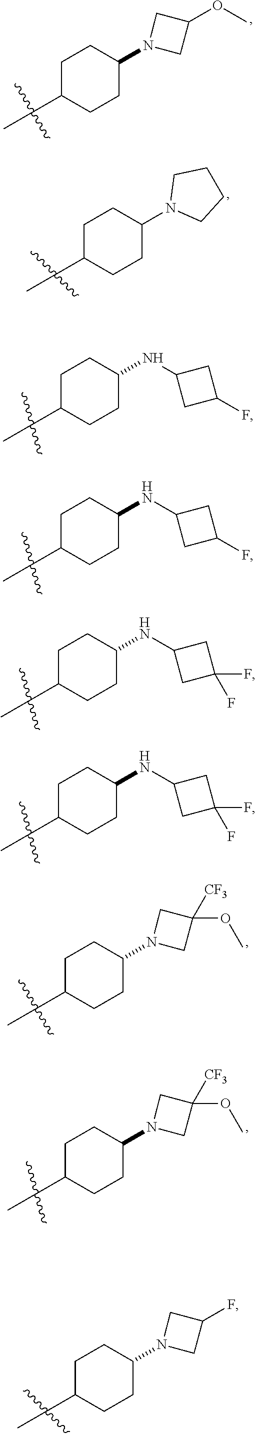

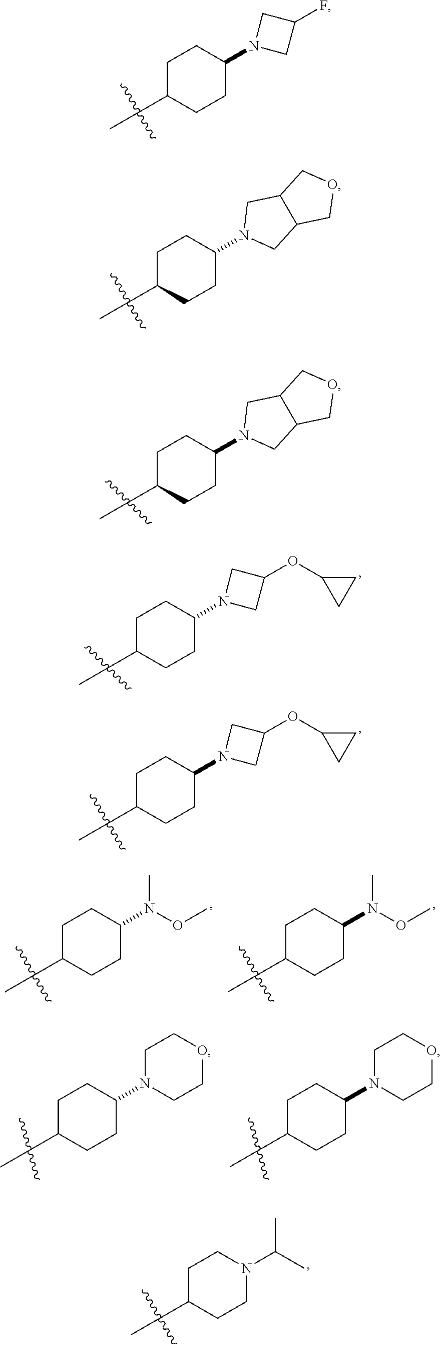

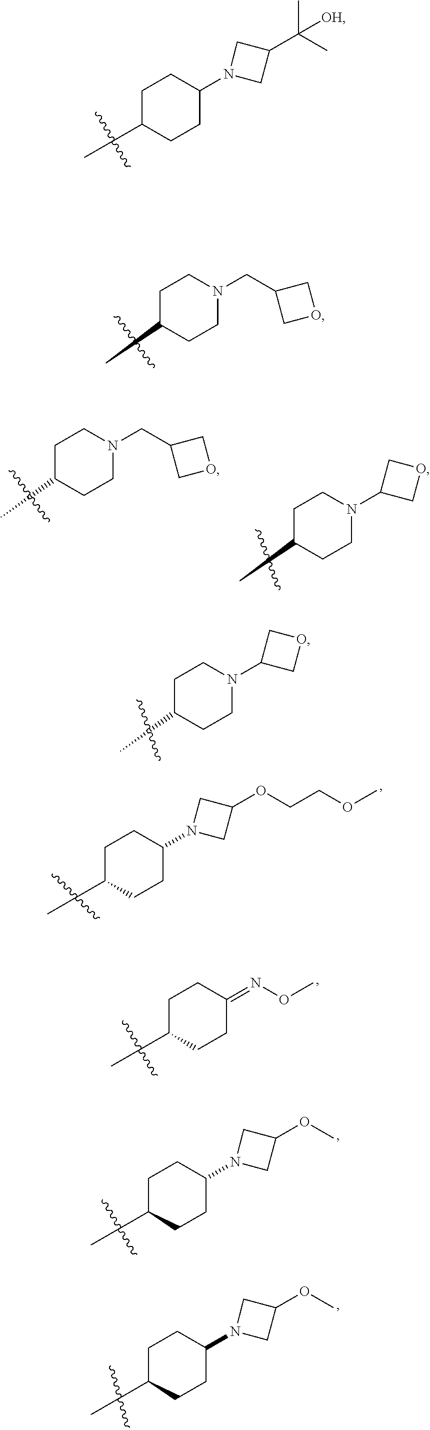

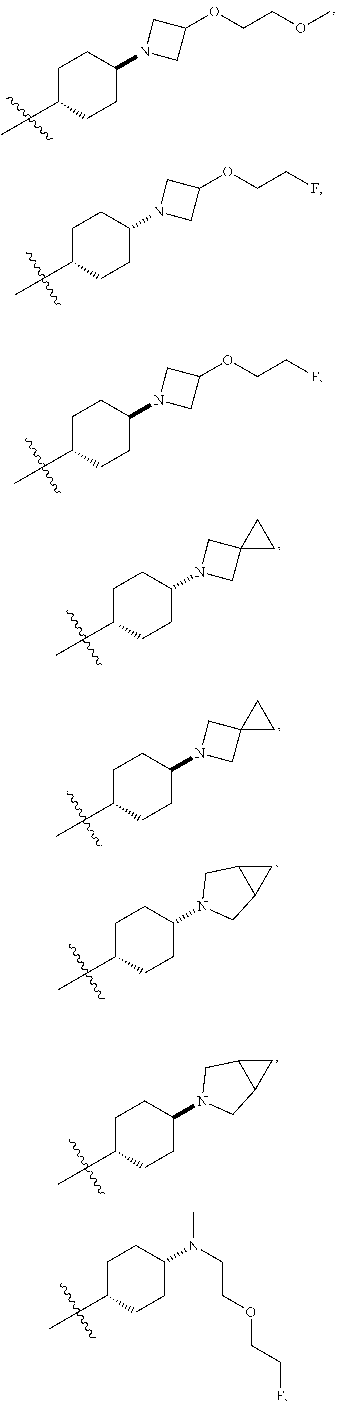

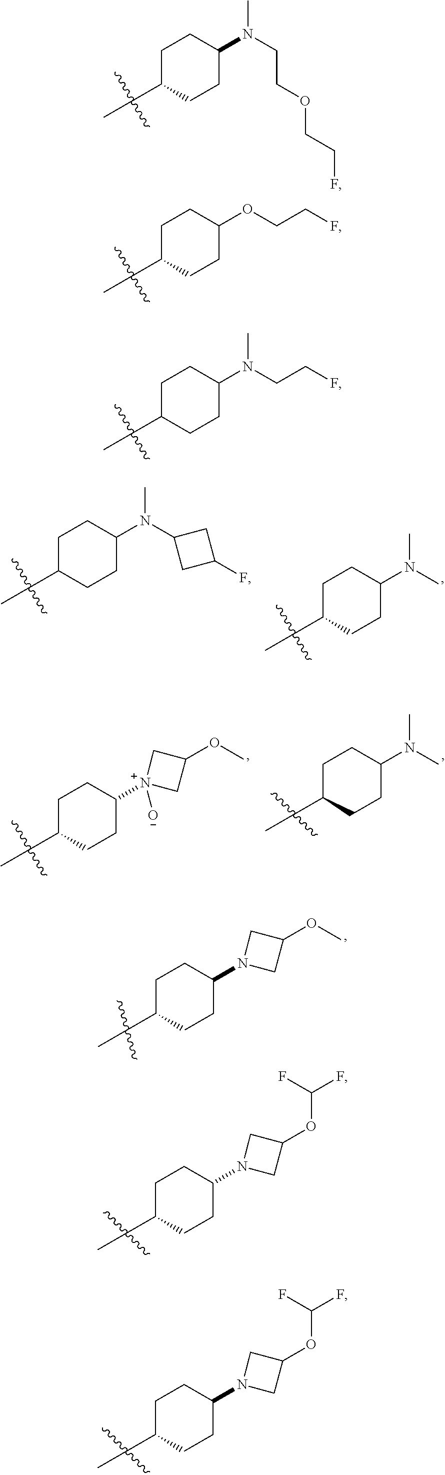

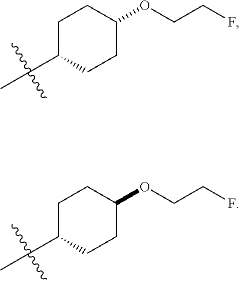 [SMILES: *[C@H]1CC[C@@H](OCCF)CC1 ; *[C@H]1CC[C@H](OCCF)CC1]

In the third embodiment of the present invention, it provides the compound of the present invention, or a deuterated compound thereof, or a stereoisomer thereof, or a pharmaceutically acceptable salt thereof, including the following compounds represented by Formula IIb:

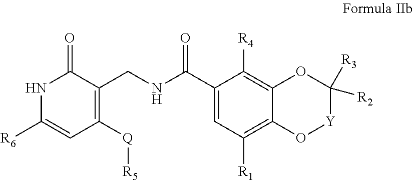

Q is selected from a group consisting of single bond, O or S; Y is selected from a group consisting of O, NR^{Y1} or CHR²;
R^{Y1} is selected from a group consisting of hydrogen, —C_{1˜6}alkyl, halo-C_{1˜6}alkyl, —C_{0˜2}alkylene-(3˜10 membered cycloalkyl), —C_{0˜2}alkylene-(3˜10 membered heterocycloalkyl), —C_{0˜2}alkylene-(5-10 membered aryl ring), —C_{0˜2}alkylene-(5-10 membered heteroaryl ring);
R^{Y2} is selected from a group consisting of hydrogen, halogen, cyano, hydroxyl, —C_{1˜6}alkyl, halo-C_{1˜6}alkyl, —O(C_{1˜6}alkyl), —O(halo-C_{1˜6}alkyl), —NH₂, —NH(C_{1˜6}alkyl), —N(C_{1˜6}alkyl) (C_{1˜6}alkyl), —C_{0˜2}alkylene-(3˜10 membered cycloalkyl), —C_{0˜2}alkylene-(3˜10 membered heterocycloalkyl), —C_{0˜2}alkylene-(5-10 membered aryl ring), —C_{0˜2}alkylene-(5-10 membered heteroaryl ring);
R₁ is selected from a group consisting of hydrogen, halogen, cyano, nitro, amino, hydroxyl, —C_{1˜6}alkyl, —O(C_{1˜6}alkyl), halo-C_{1˜6}alkyl, —C_{0˜2}alkylene-OR^{1b}, —C_{0˜2}alkylene-C(O)R^{1b}, —C_{0˜2}alkylene-C(O)NR^{1b}R^{1c}, —C_{0˜2}alkylene-NR^{1b}R^{1c}, —C_{0˜2}alkylene-NR^{1b}C(O)R^{1c}, —C_{0˜4}alkylene-S(O)₂R^{1b}, —C_{0˜2}alkylene-(3˜10 membered cycloalkyl), —C_{0˜2}alkylene-(3˜10 membered heterocycloalkyl), —C_{0˜2}alkylene-(5-10 membered aryl ring), —C_{0˜2}alkylene-(5-10 membered heteroaryl ring); wherein said alkyl, alkylene, cycloalkyl, heterocycloalkyl, aryl ring, heteroaryl ring is optionally further substituted by one, two or three independent R^{1b};
R^{1b}, R^{1c} is each independently selected from a group consisting of hydrogen, —OH, C_{1˜6}alkyl, halo-C_{1˜6}alkyl, —O(C_{1˜6}alkyl), —O(halo-C_{1˜6}alkyl), —NH₂, —NH(C_{1˜6}alkyl), —N(C_{1˜6}alkyl) (C_{1˜6}alkyl), —C_{0˜2}alkylene-(3˜10 membered cycloalkyl), —C_{0˜2}alkylene-(3˜10 membered heterocycloalkyl), —C_{0˜2}alkylene-(5-10 membered aryl ring), —C_{0˜2}alkylene-(5-10 membered heteroaryl ring);
R₂ is selected from a group consisting of —C_{0˜2}alkylene-(3-10 membered cycloalkyl), —C_{0˜2}alkylene-(3˜10 membered heterocycloalkyl); wherein said alkylene, cycloalkyl, heterocycloalkyl is optionally further substituted by one, two or three independent R^{2b};
R^{2b} is each independently selected from a group consisting of hydrogen, halogen, cyano, nitro, amino, hydroxyl, —C_{1˜6}alkyl, —O(C_{1˜6}alkyl), halo-C_{1˜6}alkyl, —C_{0˜2}alkylene-OR^{2e}, —C_{0˜2}alkylene-C(O)R^{2d}, —C_{0˜2}alkylene-C(O)NR^{2c}R^{2d}, —C_{0˜2}alkylene-NR^{2c}R^{2d}, —C_{0˜2}alkylene-NR^{2c}C(O)R^{2d}, —C_{0˜4}alkylene-S(O)₂R^{2c}, —C_{0˜2}alkylene-(3˜10 membered cycloalkyl), —C_{0˜2}alkylene-(3˜10 membered heterocycloalkyl), —C_{0˜2}alkylene-(5˜10 membered aryl ring), —C_{0˜2}alkylene-(5-10 membered heteroaryl ring); wherein alkyl, alkylene, cycloalkyl, heterocycloalkyl, aryl ring, heteroaryl ring is optionally further substituted by one, two or three independent R^{2c};
R^{2c}, R^{2d} is each independently selected from a group consisting of hydrogen, cyano, nitro, —OH, halogen, —C_{1˜6}alkyl, halo-C_{1˜6}alkyl, —C_{0˜2}alkylene-O(C_{1˜6}alkyl), —O(halo-C_{1˜6}alkyl), —O(3˜10 membered cycloalkyl), —O(3˜10 membered heterocycloalkyl), —NH₂, —NH(C_{1˜6}alkyl), —N(C_{1˜6}alkyl) (C_{1˜6}alkyl), —C_{0˜2}alkylene-(3˜10 membered cycloalkyl), —C_{0˜2}alkylene-(3˜10 membered heterocycloalkyl), —C_{0˜2}alkylene-(5˜10 membered aryl ring), —C_{0˜2}alkylene-(5˜10 membered heteroaryl ring); wherein said alkyl, alkylene, cycloalkyl, heterocycloalkyl, aryl ring, heteroaryl ring is optionally further substituted by one, two or three independent R^{2e};
R^{2e} is each independently selected from a group consisting of hydrogen, cyano, nitro, —OH, halogen, —C_{1˜6}alkyl, halo-C_{1˜6}alkyl, —C_{0˜2}alkylene-O(C_{1˜6}alkyl), —O(halo-C_{1˜6}alkyl), —O(3-10 membered cycloalkyl), —O(3˜10 membered heterocycloalkyl), —NH₂, —NH(C_{1˜6}alkyl), —N(C_{1˜6}alkyl) (C_{1˜6}alkyl), —C_{0˜2}alkylene-(3˜10 membered cycloalkyl), —C_{0˜2}alkylene-(3˜10 membered heterocycloalkyl), —C_{0˜2}alkylene-(5˜10 membered aryl ring), —C_{0˜2}alkylene-(5˜10 membered heteroaryl ring);
R₃ is selected from a group consisting of hydrogen, halogen, cyano, nitro, amino, —OH, —C_{1˜6}alkyl, —O(C_{1˜6}alkyl), halo-C_{1˜6}alkyl, —C_{0˜2}alkylene-OR^{3b}, —C_{0˜2}alkylene-C(O)R^{3b}, —C_{0˜2}alkylene-C(O)NR^{3b}R^{3c}, —C_{0˜2}alkylene-NR^{3b}R^{3c}, —C_{0˜2}alkylene-NR^{3b}C(O)R^{3c}, —C_{0˜4}alkylene-S(O)₂R^{3b}, —C_{0˜2}alkylene-(3˜10 membered cycloalkyl), —C_{0˜2}alkylene-(3˜10 membered heterocycloalkyl);
R^{3b}, R^{3c} is each independently selected from a group consisting of hydrogen, halogen, cyano, nitro, —OH, C_{1˜6}alkyl, halo-C_{1˜6}alkyl, —O(C_{1˜6}alkyl), —O(halo-C_{1˜6}alkyl), —NH₂, —NH(C_{1˜6}alkyl), —N(C_{1˜6}alkyl) (C_{1˜6}alkyl), —C_{0˜2}alkylene-(3˜10 membered cycloalkyl), —C_{0˜2}alkylene-(3˜10 membered heterocycloalkyl), —C_{0˜2}alkylene-(5-10 membered aryl ring), —C_{0˜2}alkylene-(5-10 membered heteroaryl ring);
R₄ is selected from a group consisting of hydrogen, halogen, cyano, nitro, hydroxyl, —C_{1˜6}alkyl, halo-C_{1˜6}alkyl, —O(C_{1˜6}alkyl), —O(halo-C_{1˜6}alkyl), —NH₂, —NH(C_{1˜6}alkyl), —N(C_{1˜6}alkyl) (C_{1˜6}alkyl), —C_{0˜2}alkylene-(3˜10 membered cycloalkyl), —C_{0˜2}alkylene-(3˜10 membered heterocycloalkyl), —C_{0˜2}alkylene-(5˜10 membered aryl ring), —C_{0˜2}alkylene-(5˜10 membered heteroaryl ring); wherein said alkyl, alkylene, cycloalkyl, heterocycloalkyl, aryl ring, heteroaryl ring is optionally further substituted by one, two or three independent R^{4b};
R^{4b} is each independently selected from a group consisting of hydrogen, halogen, cyano, nitro, —OH, C_{1˜6}alkyl, halo-C_{1˜6}alkyl, —O(C_{1˜6}alkyl), —O(halo-C_{1˜6}alkyl), —NH₂, —NH(C_{1˜6}alkyl), —N(C_{1˜6}alkyl) (C_{1˜6}alkyl), —C_{0˜2}alkylene-(3˜10 membered cycloalkyl), —C_{0˜2}alkylene-(3˜10 membered heterocycloalkyl), —C_{0˜2}alkylene-(5-10 membered aryl ring), —C_{0˜2}alkylene-(5-10 membered heteroaryl ring);
R₈ is selected from a group consisting of hydrogen, halogen, cyano, nitro, hydroxyl, —C_{1˜6}alkyl, halo-C_{1˜6}alkyl, —O(C_{1˜6}alkyl), —O(halo-C_{1˜6}alkyl), —NH₂, —NH(C_{1˜6}alkyl), —N(C_{1˜6}alkyl) (C_{1˜6}alkyl), —C_{0˜2}alkylene-(3˜10 membered cycloalkyl), —C_{0˜2}alkylene-(3˜10 membered heterocycloalkyl), —C_{0˜2}alkylene-(5-10 membered aryl ring), —C_{0˜2}alkylene-(5-10 membered heteroaryl ring);
R₆ is selected from a group consisting of hydrogen, halogen, cyano, nitro, hydroxyl, —C_{1˜6}alkyl, halo-C_{1˜6}alkyl, —O(C_{1˜6}alkyl), —O(halo-C_{1˜6}alkyl), —NH₂, —NH(C_{1˜6}alkyl), —N(C_{1˜6}alkyl) (C_{1˜6}alkyl), —C_{0˜2}alkylene-(3˜10 membered cycloalkyl), —C_{0˜2}alkylene-(3˜10 membered heterocycloalkyl), —C_{0˜2}alkylene-(5-10 membered aryl ring), —C_{0˜2}alkylene-(5-10 membered heteroaryl ring);

In the third embodiment of the present invention, it provides the compound of the present invention, or a deuterated compound thereof, or a stereoisomer thereof, or a pharmaceutically acceptable salt thereof, wherein said R₁, R₃, R₄, R₅, R₆ is each independently selected from a group consisting of hydrogen, halogen, —C_{1˜3}alkyl;
Q is selected from a group consisting of single bond; Y is selected from a group consisting of CHR²; R¹² is selected from a group consisting of hydrogen, —C_{1˜3}alkyl.

In the third embodiment of the present invention, it provides the compound of the present invention, or a deuterated compound thereof, or a stereoisomer thereof, or a pharmaceutically acceptable salt thereof, wherein said R₂ is specifically selected from a group consisting of

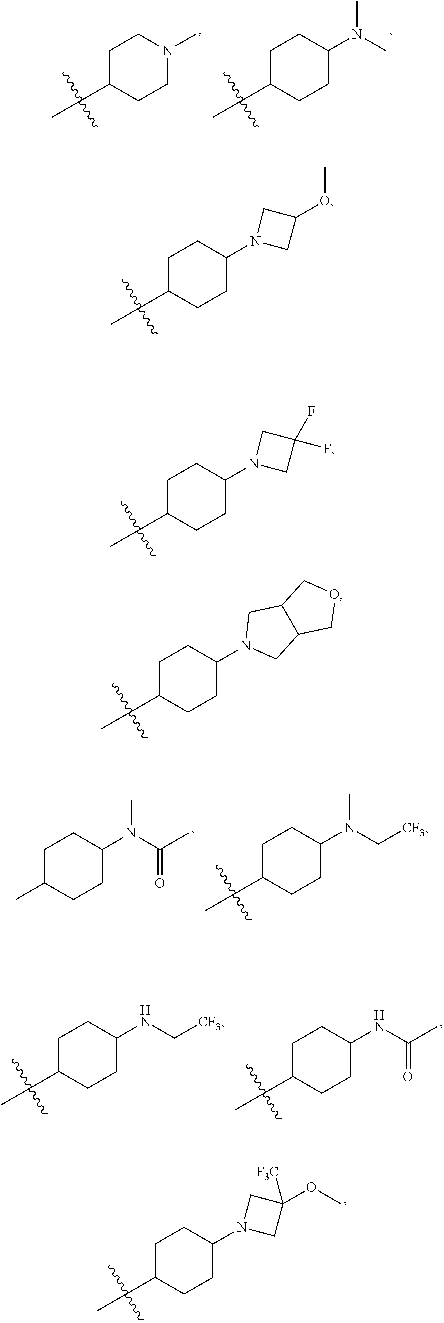

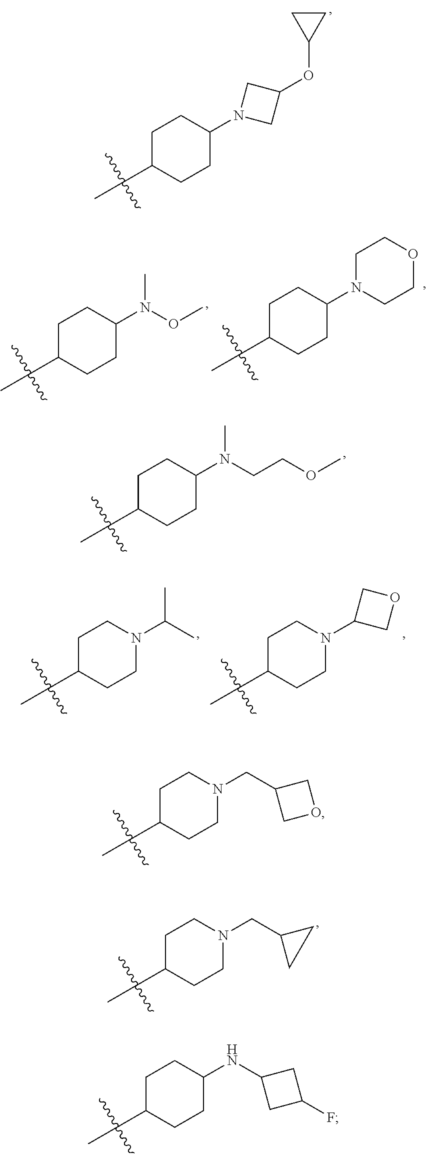

preferably, said R₂ is specifically selected from a group consisting of

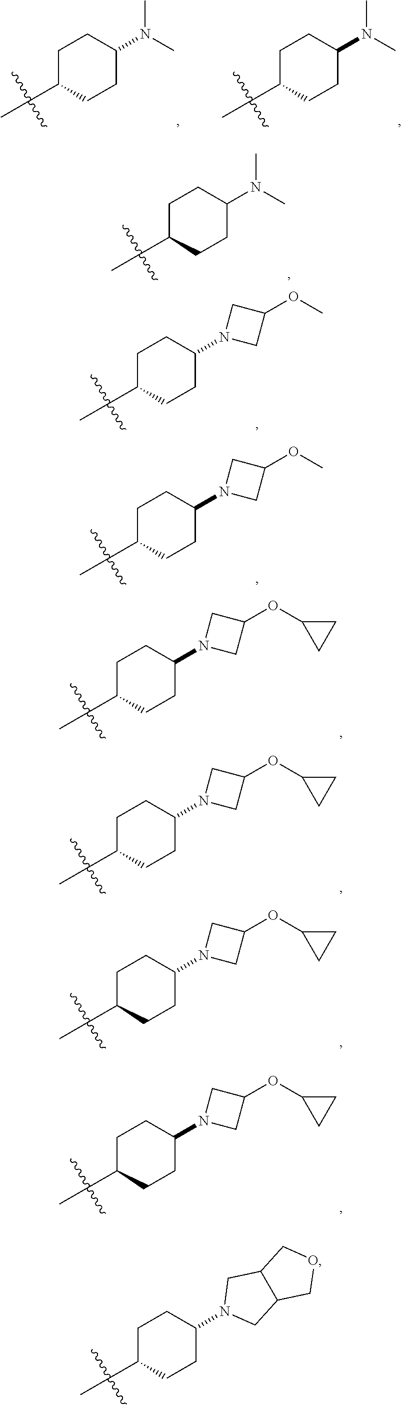

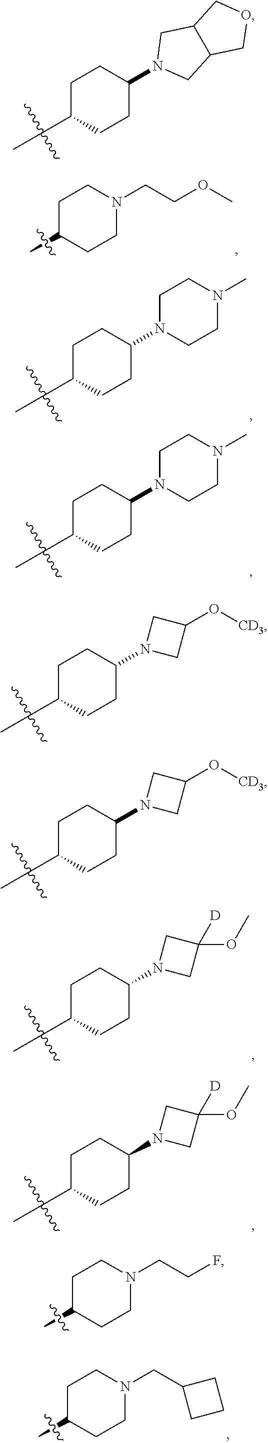

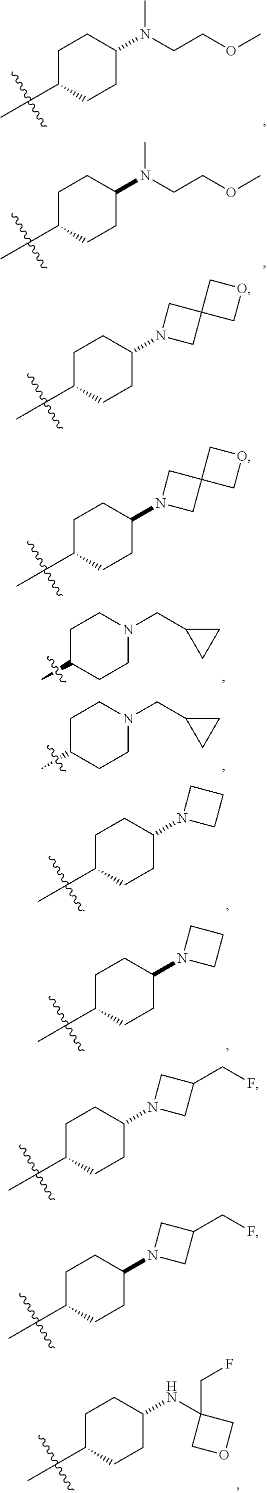

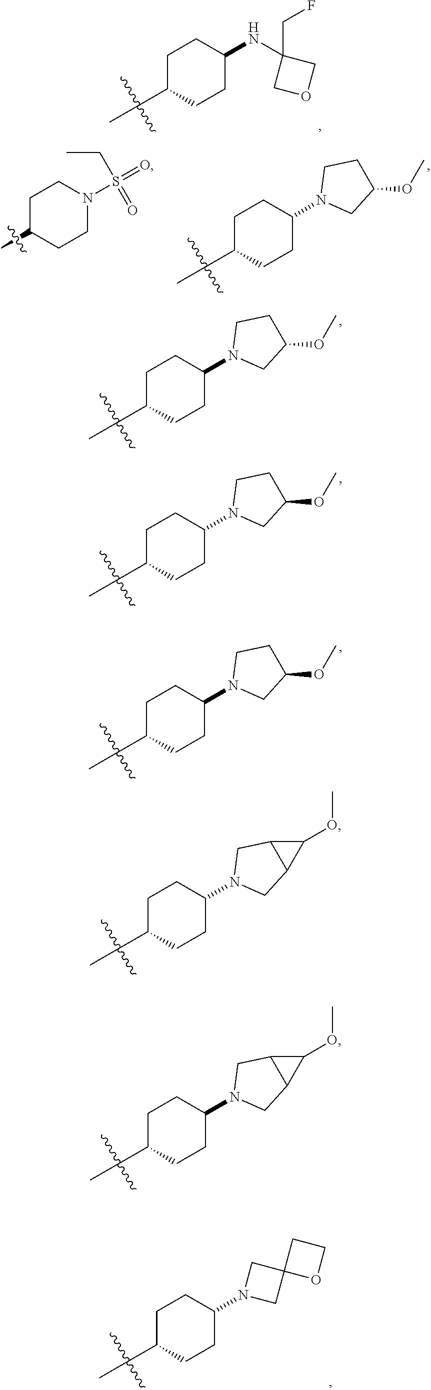

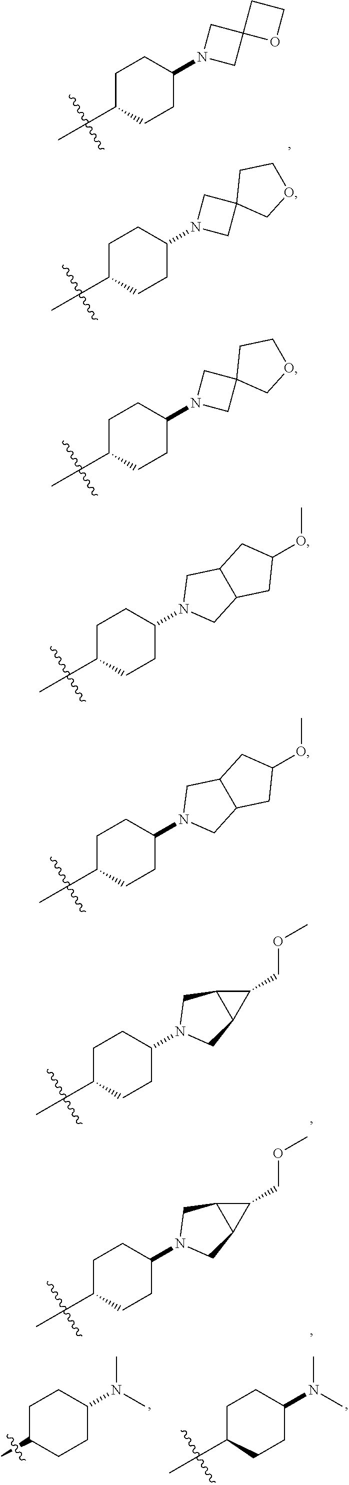

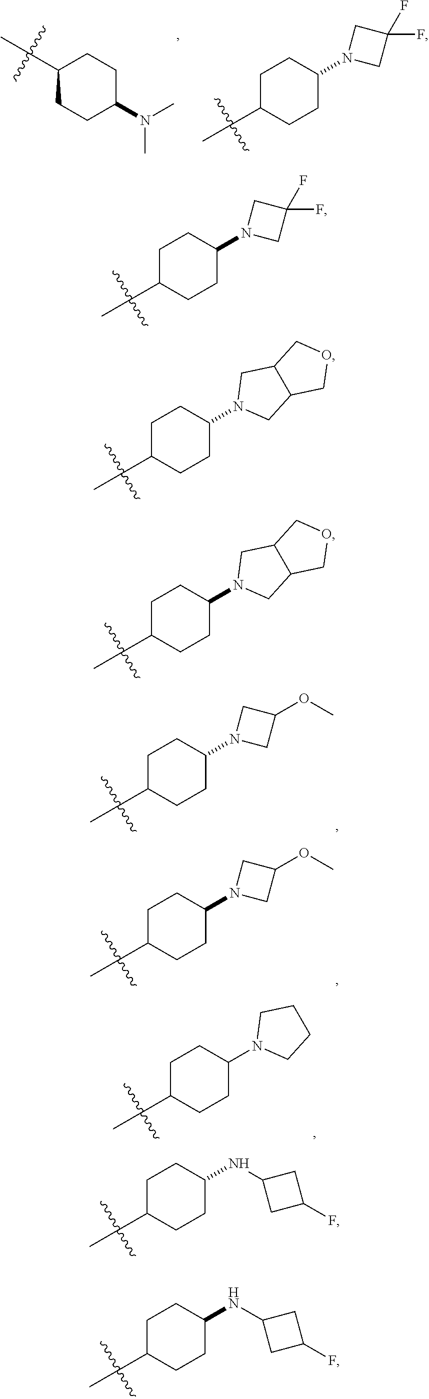

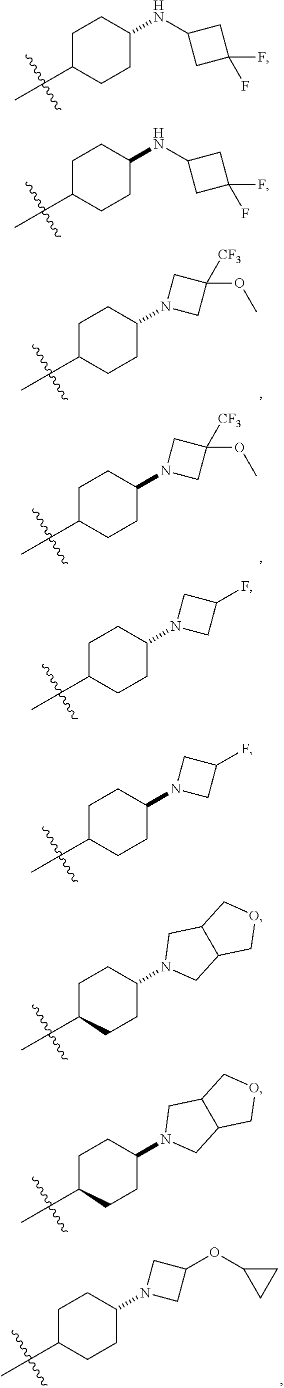

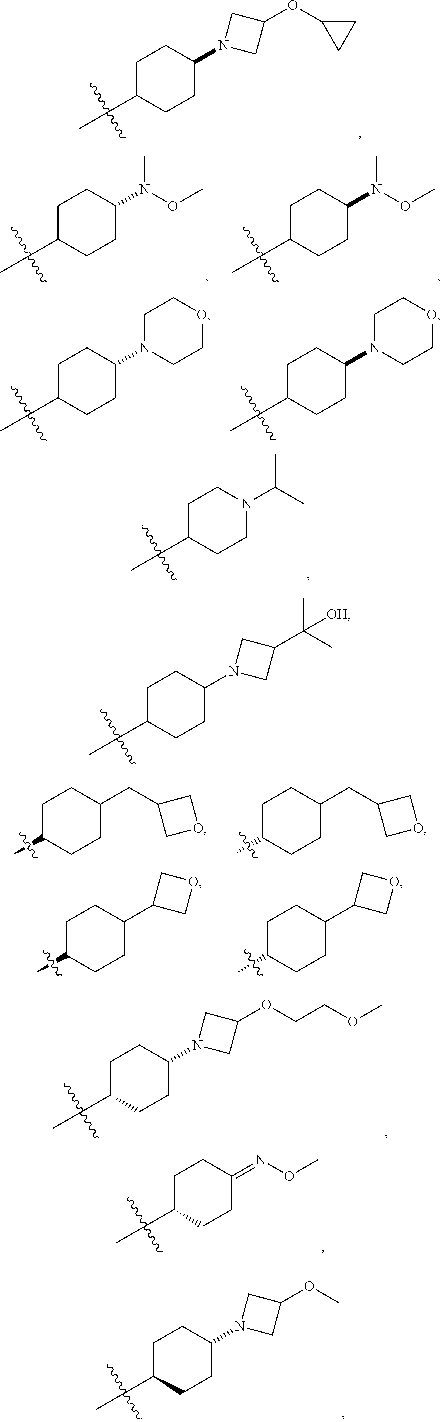

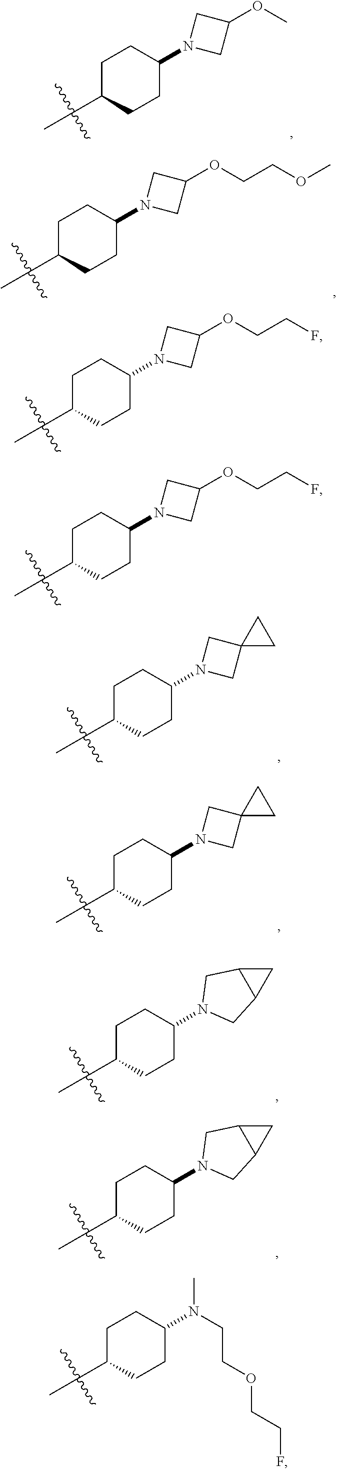

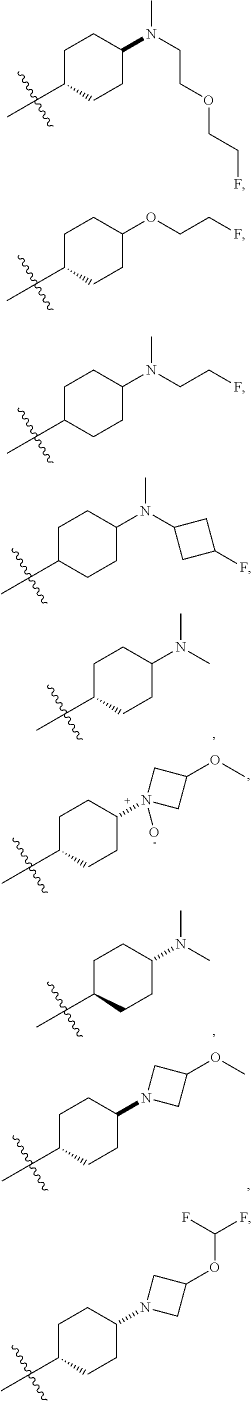

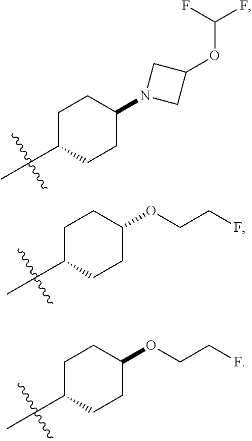 [SMILES: *[C@H]1CC[C@H](N2CC(OC(F)F)C2)CC1 ; *[C@H]1CC[C@@H](OCCF)CC1 ; *[C@H]1CC[C@H](OCCF)CC1]

In the fourth embodiment of the present invention, it provides the compound of the present invention, or a deuterated compound thereof, or a stereoisomer thereof, or a pharmaceutically acceptable salt thereof, including the following compounds represented by Formula IIc:

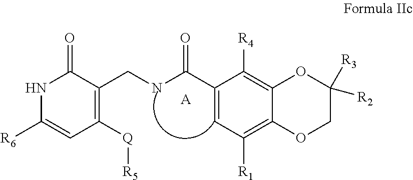

Q is selected from a group consisting of single bond, O or S;
A-ring is selected from a group consisting of 4-10 membered heterocyclic ring; wherein said heterocyclic ring is optionally further substituted by one, two or three independent R⁷¹;
each R⁷¹ is each independently selected from a group consisting of hydrogen, halogen, cyano, nitro, —OH, C_{1˜6}alkyl, halo-C_{1˜6}alkyl, —O(C_{1˜6}alkyl), —O(halo-C_{1˜6}alkyl), —NH₂, —NH(C_{1˜6}alkyl), —N(C_{1˜6}alkyl) (C_{1˜6}alkyl), —C_{0˜2}alkylene-(3˜10 membered cycloalkyl), —C_{0˜2}alkylene-(3˜10 membered heterocycloalkyl), —C_{0˜2}alkylene-(5˜10 membered aryl ring), —C_{0˜2}alkylene-(5˜10 membered heteroaryl ring);
R₁ is selected from a group consisting of hydrogen, halogen, cyano, nitro, amino, hydroxyl, —C_{1˜6}alkyl, —O(C_{1˜6}alkyl), halo-C_{1˜6}alkyl, —C_{0˜2}alkylene-OR^{1b}, —C_{0˜2}alkylene-C(O)R^{1b}, —C_{0˜2}alkylene-C(O)NR^{1b}R^{1c}, —C_{0˜2}alkylene-NR^{1b}R^{1c}, —C_{0˜2}alkylene-NR^{1b}C(O)R^{1c}, —C_{0˜4}alkylene-S(O)₂R^{1b}R^{1c}, —C_{0˜2}alkylene-(3˜10 membered cycloalkyl), —C_{0˜2}alkylene-(3˜10 membered heterocycloalkyl), —C_{0˜2}alkylene-(5-10 membered aryl ring), —C_{0˜2}alkylene-(5-10 membered heteroaryl ring); wherein said alkyl, alkylene, cycloalkyl, heterocycloalkyl, aryl ring, heteroaryl ring is optionally further substituted by one, two or three independent R^{1b};
R^{1b}, R^{1c} is each independently selected from a group consisting of hydrogen, —OH, C_{1˜6}alkyl, halo-C_{1˜6}alkyl, —O(C_{1˜6}alkyl), —O(halo-C_{1˜6}alkyl), —NH₂, —NH(C_{1˜6}alkyl), —N(C_{1˜6}alkyl) (C_{1˜6}alkyl), —C_{0˜2}alkylene-(3˜10 membered cycloalkyl), —C_{0˜2}alkylene-(3˜10 membered heterocycloalkyl), —C_{0˜2}alkylene-(5-10 membered aryl ring), —C_{0˜2}alkylene-(5-10 membered heteroaryl ring);
R₂ is selected from a group consisting of —C_{0˜2}alkylene-(3-10 membered cycloalkyl), —C_{0˜2}alkylene-(3˜10 membered heterocycloalkyl); wherein said alkylene, cycloalkyl, heterocycloalkyl is optionally further substituted by one, two or three independent R^{2b};
R^{2b} is each independently selected from a group consisting of hydrogen, halogen, cyano, nitro, amino, hydroxyl, —C_{1˜6}alkyl, —O(C_{1˜6}alkyl), halo-C_{1˜6}alkyl, —C_{0˜2}alkylene-OR^{2e}, —C_{0˜2}alkylene-C(O)R^{2c}, —C_{0˜2}alkylene-C(O)NR^{2c}R^{2d}, —C_{0˜2}alkylene-NR^{2c}R^{2d}, —C_{0˜2}alkylene-NR^{2c}C(O)R^{2d}, —C_{0˜4}alkylene-S(O)₂R^{2c}, —C_{0˜2}alkylene-(3˜10 membered cycloalkyl), —C_{0˜2}alkylene-(3˜10 membered heterocycloalkyl), —C_{0˜2}alkylene-(5˜10 membered aryl ring), —C_{0˜2}alkylene-(5-10 membered heteroaryl ring); wherein alkyl, alkylene, cycloalkyl, heterocycloalkyl, aryl ring, heteroaryl ring is optionally further substituted by one, two or three independent R^{2c};
R^{2c}, R^{2d} is each independently selected from a group consisting of hydrogen, cyano, nitro, —OH, halogen, —C_{1˜6}alkyl, halo-C_{1˜6}alkyl, —C_{0˜2}alkylene-O(C_{1˜6}alkyl), —O(halo-C_{1˜6}alkyl), —O(3-10 membered cycloalkyl), —O(3˜10 membered heterocycloalkyl), —NH₂, —NH(C_{1˜6}alkyl), —N(C_{1˜6}alkyl) (C_{1˜6}alkyl), —C_{0˜2}alkylene-(3˜10 membered cycloalkyl), —C_{0˜2}alkylene-(3˜10 membered heterocycloalkyl), —C_{0˜2}alkylene-(5˜10 membered aryl ring), —C_{0˜2}alkylene-(5˜10 membered heteroaryl ring); wherein said alkyl, alkylene, cycloalkyl, heterocycloalkyl, aryl ring, heteroaryl ring is optionally further substituted by one, two or three independent R^{2e};
R^{2e} is each independently selected from a group consisting of hydrogen, cyano, nitro, —OH, halogen, —C_{1˜6}alkyl, halo-C_{1˜6}alkyl, —C_{0˜2}alkylene-O(C_{1˜6}alkyl), —O(halo-C_{1˜6}alkyl), —O(3˜10 membered cycloalkyl), —O(3˜10 membered heterocycloalkyl), —NH₂, —NH(C_{1˜6}alkyl), —N(C_{1˜6}alkyl) (C_{1˜6}alkyl), —C_{0˜2}alkylene-(3˜10 membered cycloalkyl), —C_{0˜2}alkylene-(3˜10 membered heterocycloalkyl), —C_{0˜2}alkylene-(5˜10 membered aryl ring), —C_{0˜2}alkylene-(5˜10 membered heteroaryl ring);
R₃ is selected from a group consisting of hydrogen, halogen, cyano, nitro, amino, —OH, —C_{1˜6}alkyl, —O(C_{1˜6}alkyl), halo-C_{1˜6}alkyl, —C_{0˜2}alkylene-OR^{3b}, —C_{0˜2}alkylene-C(O)R^{3b}, —C_{0˜2}alkylene-C(O)NR^{3b}R^{3c}, —C_{0˜2}alkylene-NR^{3b}R^{3c}, —C_{0˜2}alkylene-NR^{3b}C(O)R^{3c}, —C_{0˜4}alkylene-S(O)₂R^{3b}, —C_{0˜2}alkylene-(3˜10 membered cycloalkyl), —C_{0˜2}alkylene-(3˜10 membered heterocycloalkyl);
R^{3b}, R^{3c} is each independently selected from a group consisting of hydrogen, halogen, cyano, nitro, —OH, C_{1˜6}alkyl, halo-C_{1˜6}alkyl, —O(C_{1˜6}alkyl), —O(halo-C_{1˜6}alkyl), —NH₂, —NH(C_{1˜6}alkyl), —N(C_{1˜6}alkyl) (C_{1˜6}alkyl), —C_{0˜2}alkylene-(3˜10 membered cycloalkyl), —C_{0˜2}alkylene-(3˜10 membered heterocycloalkyl), —C_{0˜2}alkylene-(5-10 membered aryl ring), —C_{0˜2}alkylene-(5-10 membered heteroaryl ring);
R₄ is selected from a group consisting of hydrogen, halogen, cyano, nitro, hydroxyl, —C_{1˜6}alkyl, halo-C_{1˜6}alkyl, —O(C_{1˜6}alkyl), —O(halo-C_{1˜6}alkyl), —NH₂, —NH(C_{1˜6}alkyl), —N(C_{1˜6}alkyl) (C_{1˜6}alkyl), —C_{0˜2}alkylene-(3˜10 membered cycloalkyl), —C_{0˜2}alkylene-(3˜10 membered heterocycloalkyl), —C_{0˜2}alkylene-(5˜10 membered aryl ring), —C_{0˜2}alkylene-(5˜10 membered heteroaryl ring); wherein said alkyl, alkylene, cycloalkyl, heterocycloalkyl, aryl ring, heteroaryl ring is optionally further substituted by one, two or three independent R^{4b};
R^{4b} is each independently selected from a group consisting of hydrogen, halogen, cyano, nitro, —OH, C_{1˜6}alkyl, halo-C_{1˜6}alkyl, —O(C_{1˜6}alkyl), —O(halo-C_{1˜6}alkyl), —NH₂, —NH(C_{1˜6}alkyl), —N(C_{1˜6}alkyl) (C_{1˜6}alkyl), —C_{0˜2}alkylene-(3˜10 membered cycloalkyl), —C_{0˜2}alkylene-(3˜10 membered heterocycloalkyl), —C_{0˜2}alkylene-(5-10 membered aryl ring), —C_{0˜2}alkylene-(5-10 membered heteroaryl ring);
R₈ is selected from a group consisting of hydrogen, halogen, cyano, nitro, hydroxyl, —C_{1˜6}alkyl, halo-C_{1˜6}alkyl, —O(C_{1˜6}alkyl), —O(halo-C_{1˜6}alkyl), —NH₂, —NH(C_{1˜6}alkyl), —N(C_{1˜6}alkyl) (C_{1˜6}alkyl), —C_{0˜2}alkylene-(3˜10 membered cycloalkyl), —C_{0˜2}alkylene-(3˜10 membered heterocycloalkyl), —C_{0˜2}alkylene-(5-10 membered aryl ring), —C_{0˜2}alkylene-(5-10 membered heteroaryl ring);
R₆ is selected from a group consisting of hydrogen, halogen, cyano, nitro, hydroxyl, —C_{1˜6}alkyl, halo-C_{1˜6}alkyl, —O(C_{1˜6}alkyl), —O(halo-C_{1˜6}alkyl), —NH₂, —NH(C_{1˜6}alkyl), —N(C_{1˜6}alkyl) (C_{1˜6}alkyl), —C_{0˜2}alkylene-(3˜10 membered cycloalkyl), —C_{0˜2}alkylene-(3˜10 membered heterocycloalkyl), —C_{0˜2}alkylene-(5-10 membered aryl ring), —C_{0˜2}alkylene-(5-10 membered heteroaryl ring); In the fourth embodiment of the present invention, it provides the compound of the present invention, or a deuterated compound thereof, or a stereoisomer thereof, or a pharmaceutically acceptable salt thereof, wherein said R₁, R₃, R₄, R₅, R₆ is each independently selected from a group consisting of hydrogen, halogen, —C_{1˜3}alkyl; Q is selected from a group consisting of single bond.

In the fourth embodiment of the present invention, it provides the compound of the present invention, or a deuterated compound thereof, or a stereoisomer thereof, or a pharmaceutically acceptable salt thereof, wherein said R₂ is specifically selected from a group consisting of

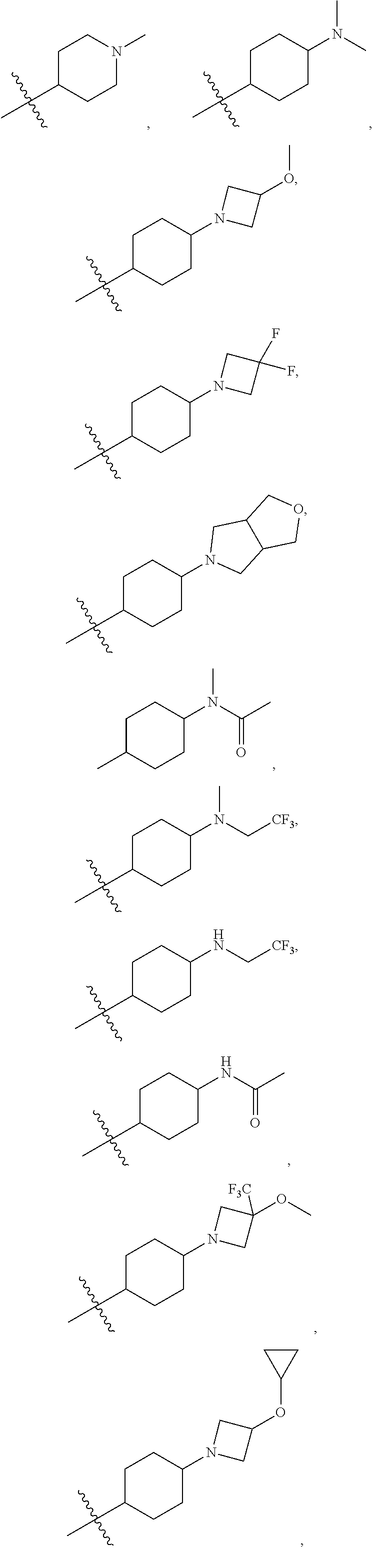

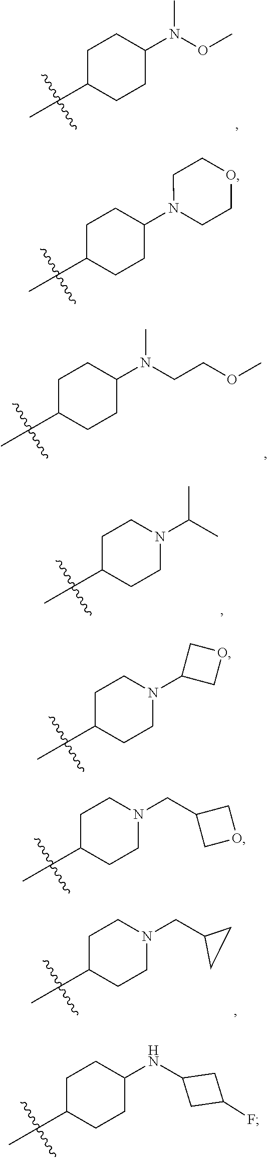

preferably, said R₂ is specifically selected from a group consisting of

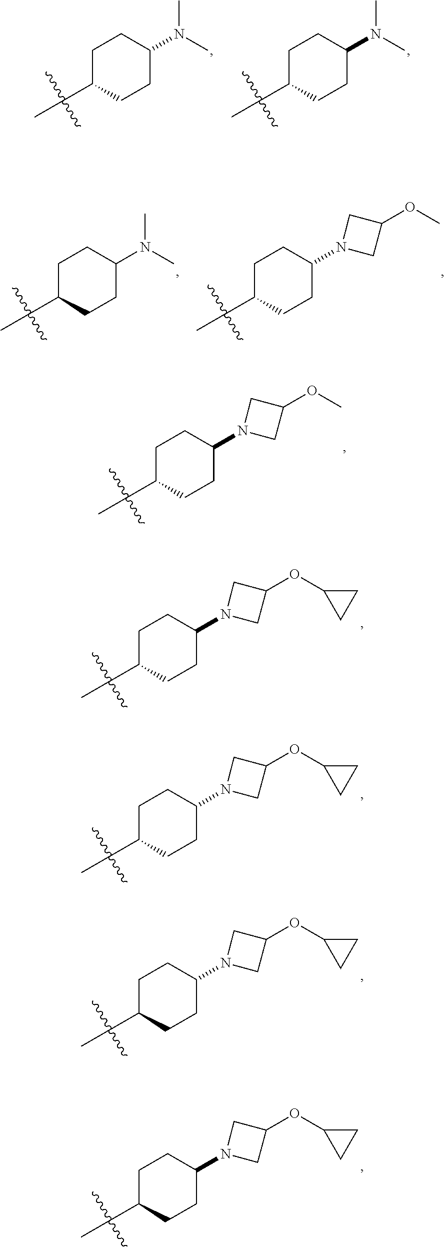

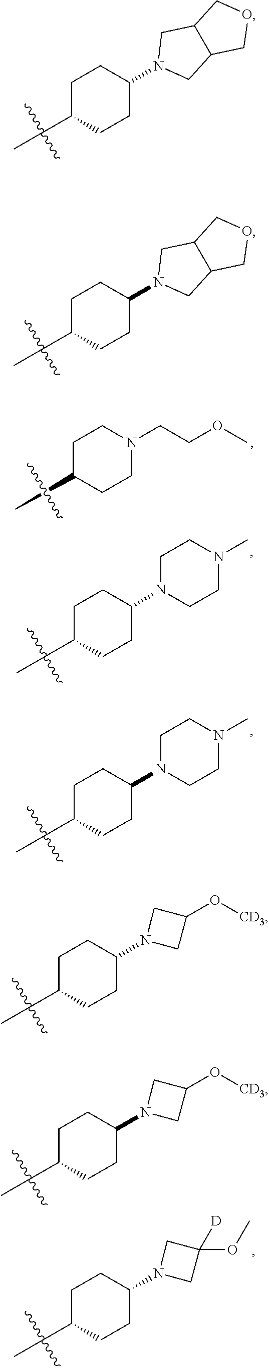

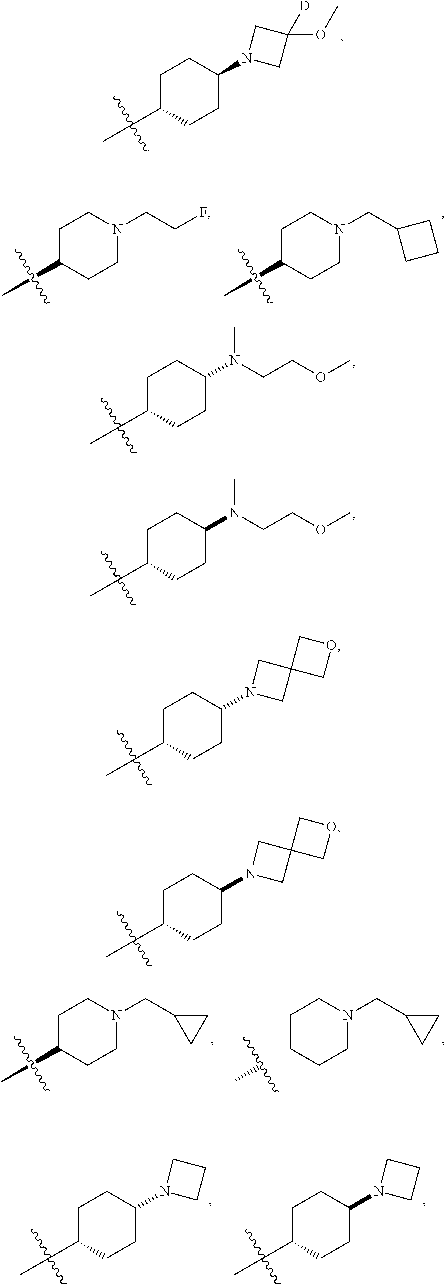

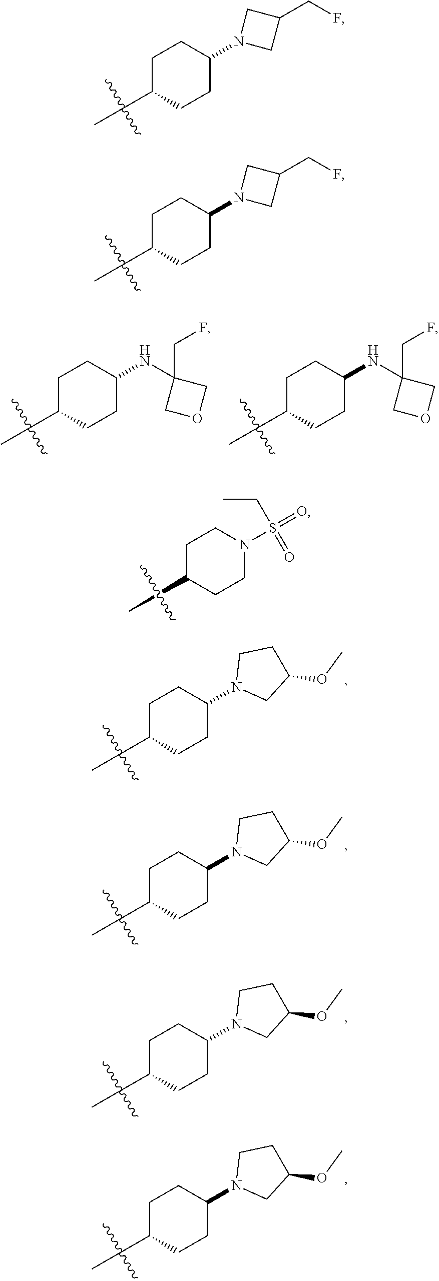

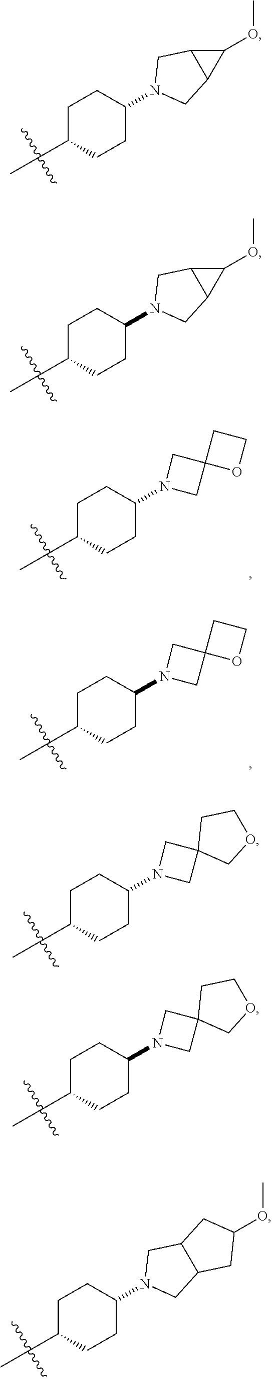

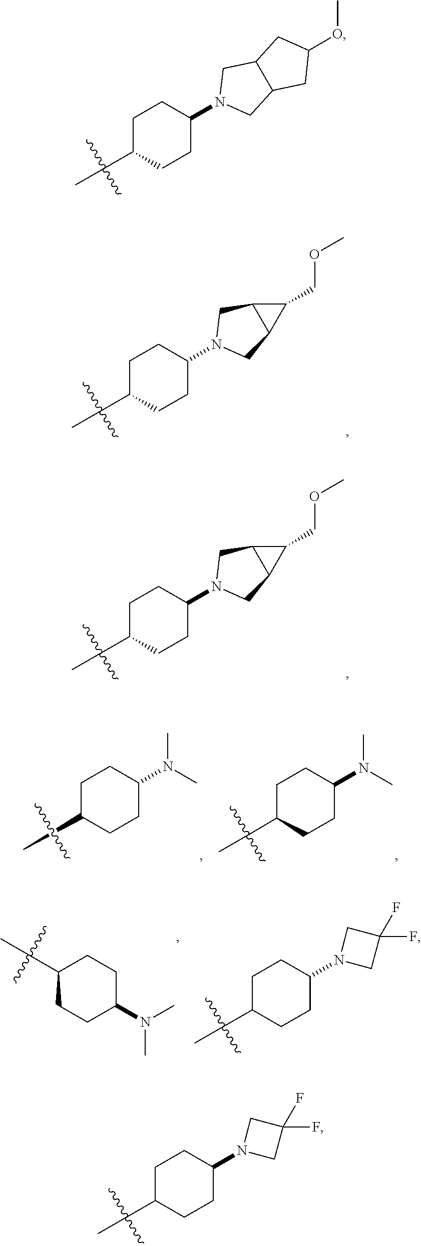

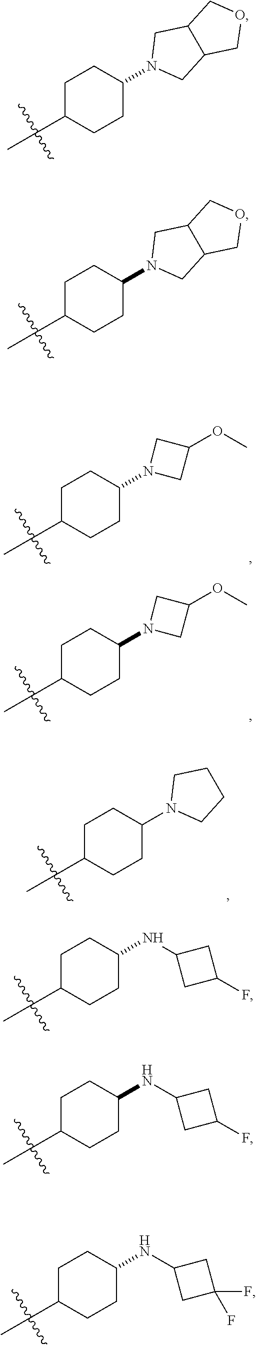

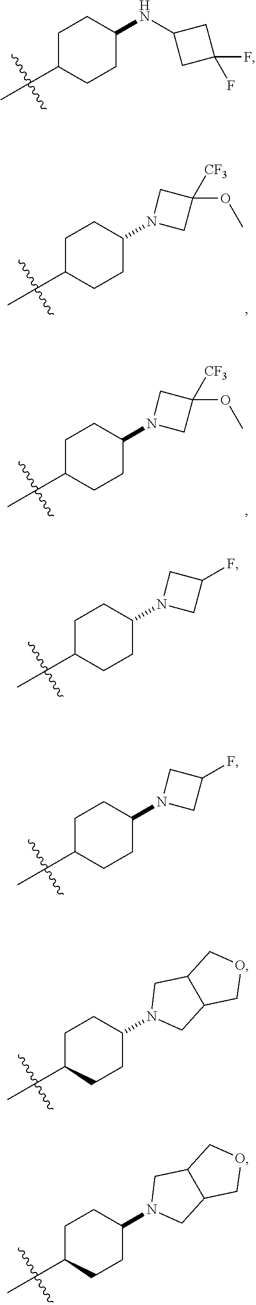

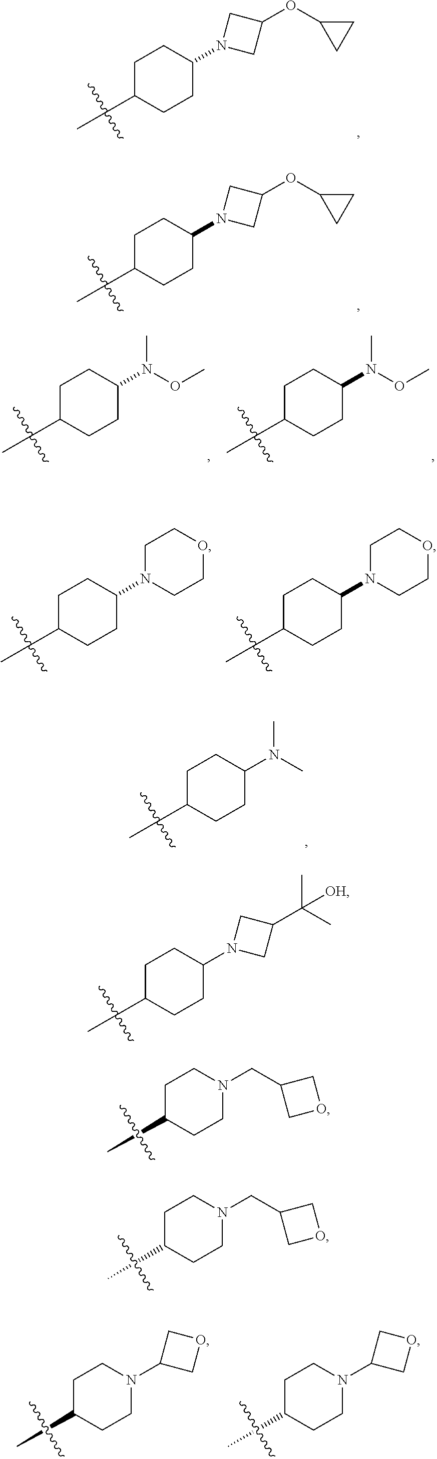

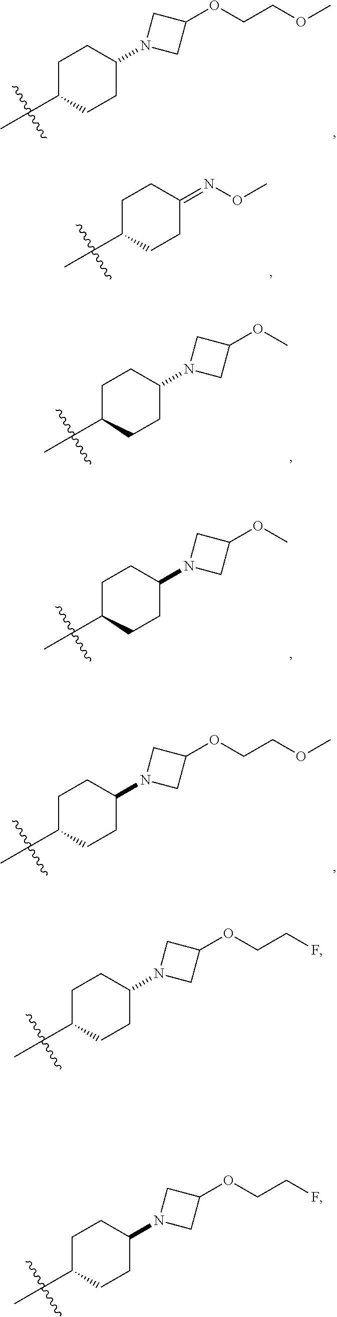

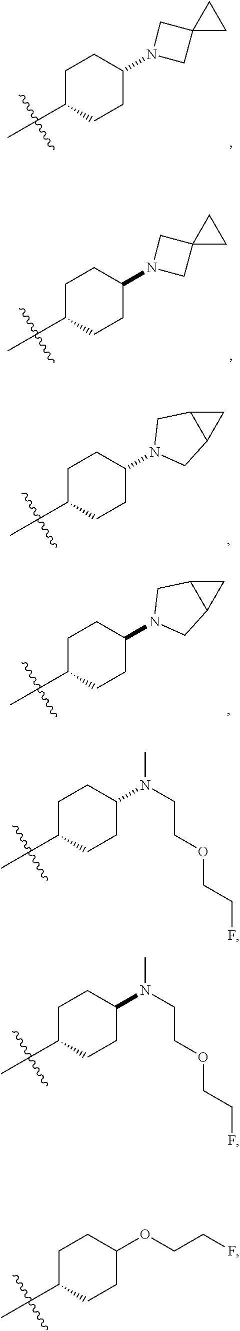

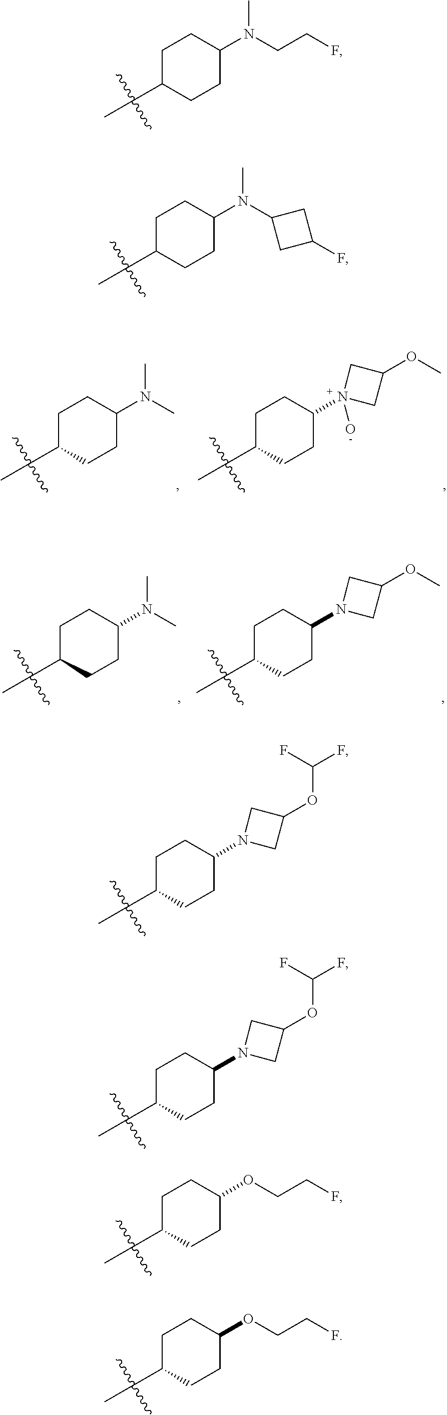

In the embodiment of the present invention, it provides the compound of the present invention, or a deuterated compound thereof, or a stereoisomer thereof, or a pharmaceutically acceptable salt thereof, wherein the compound represented by Formula I or Formula II is specifically

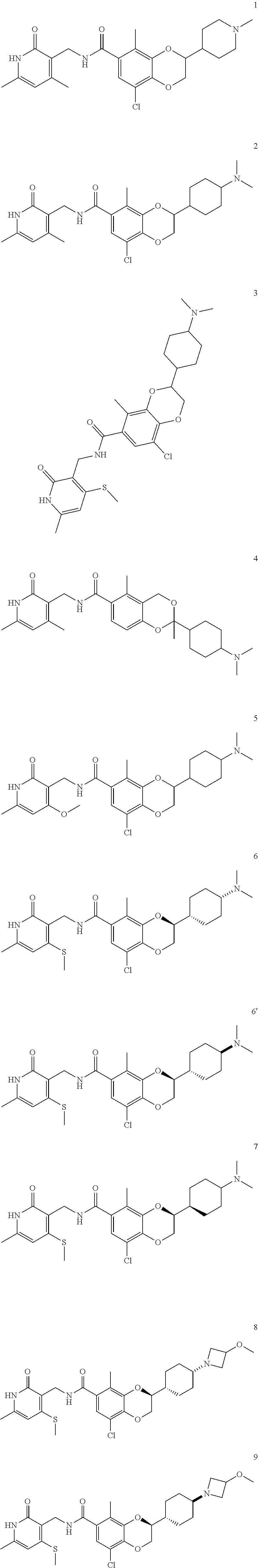

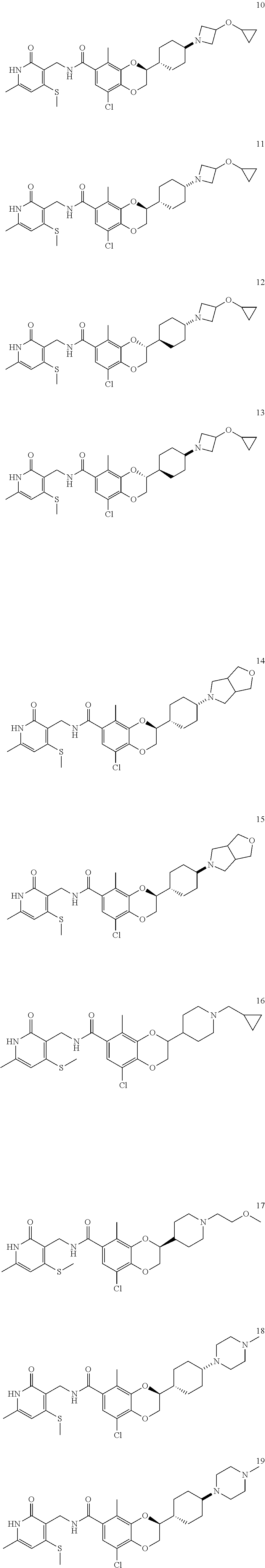

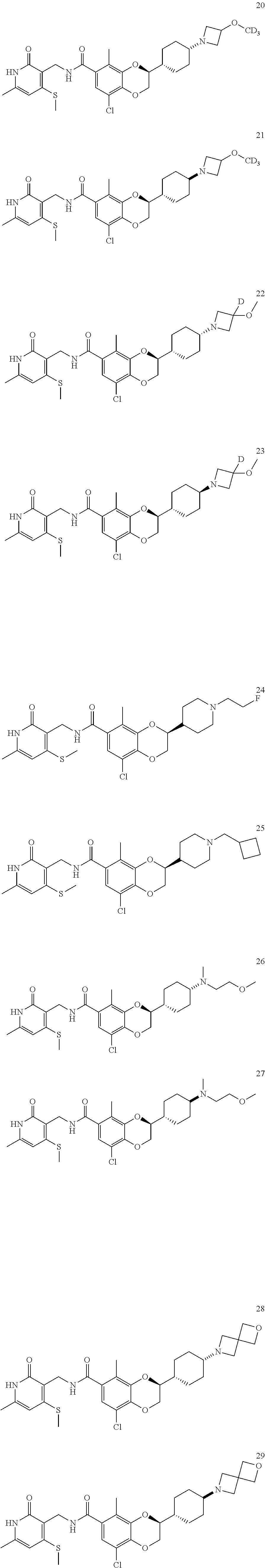

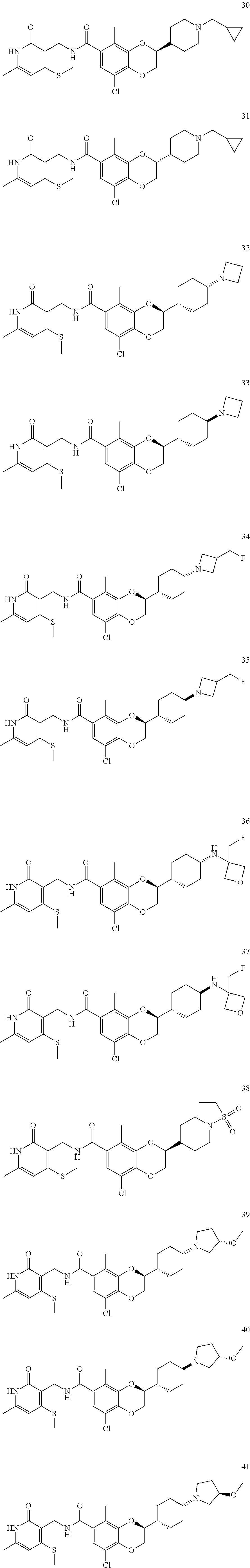

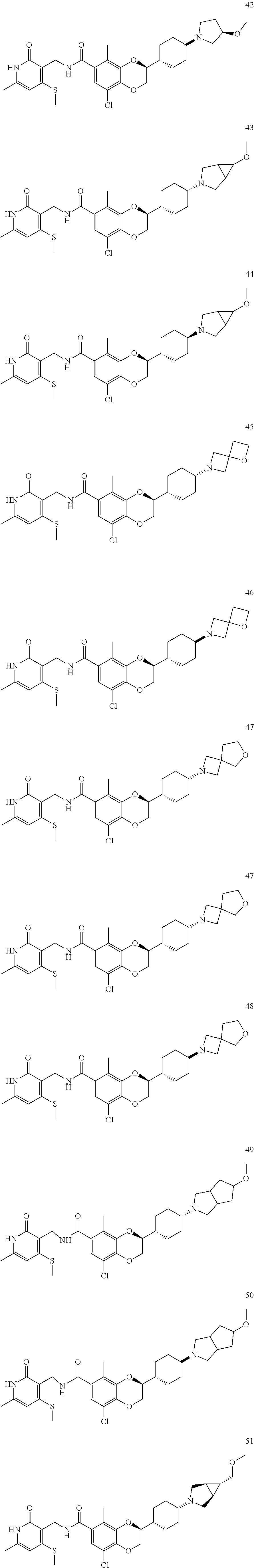

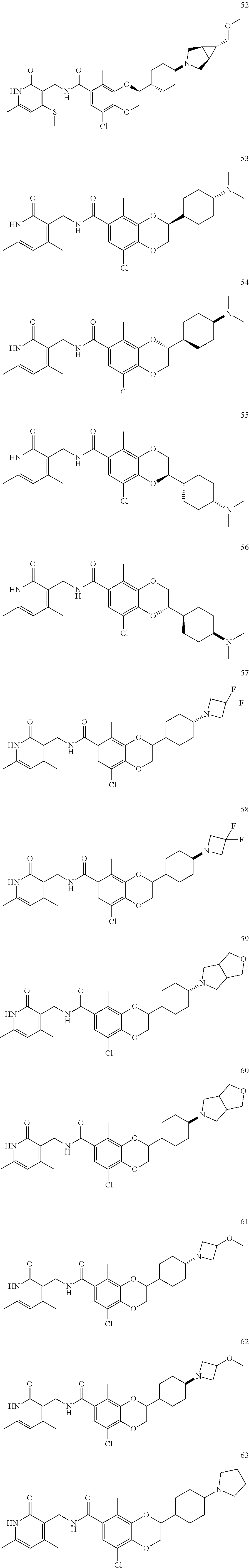

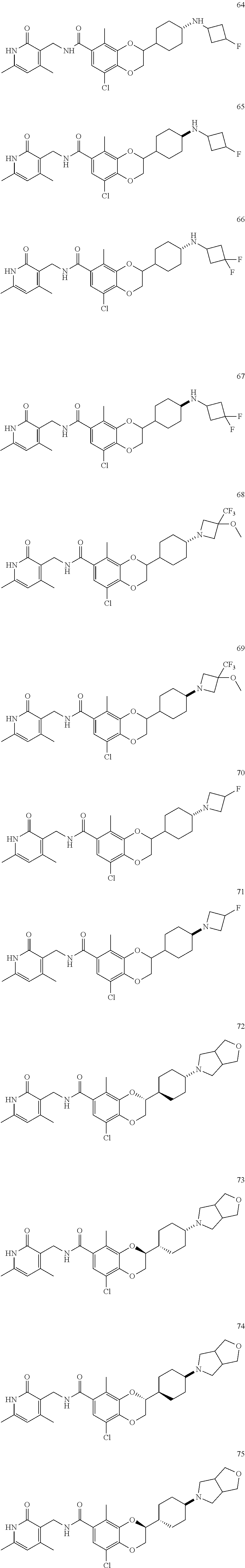

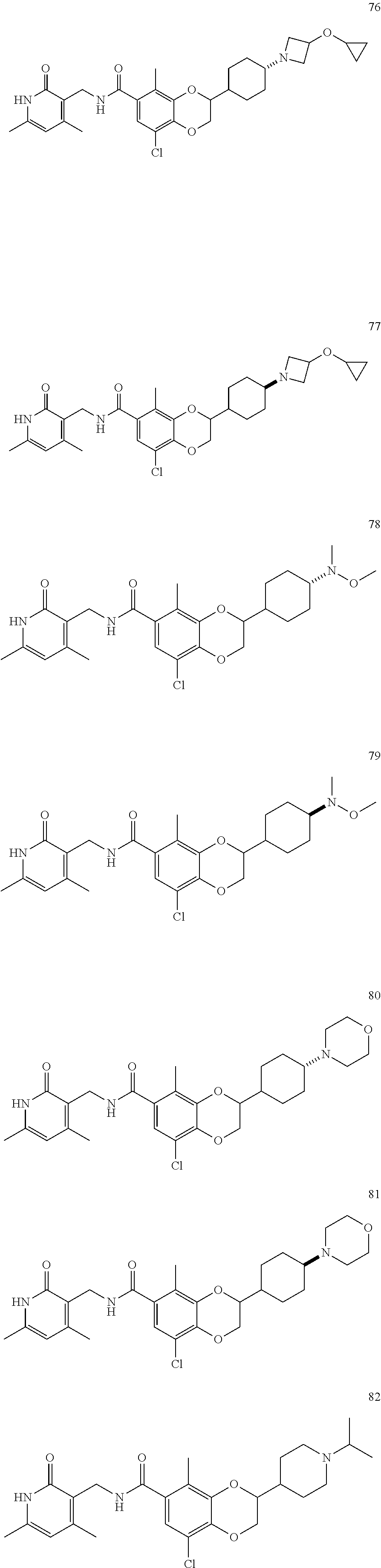

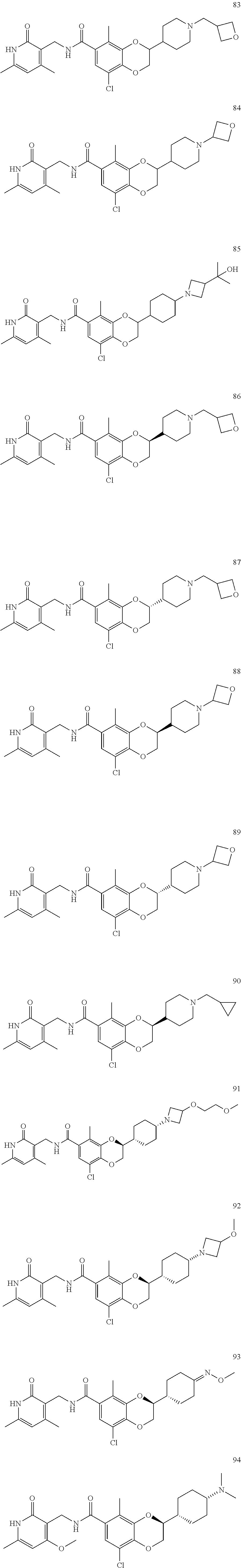

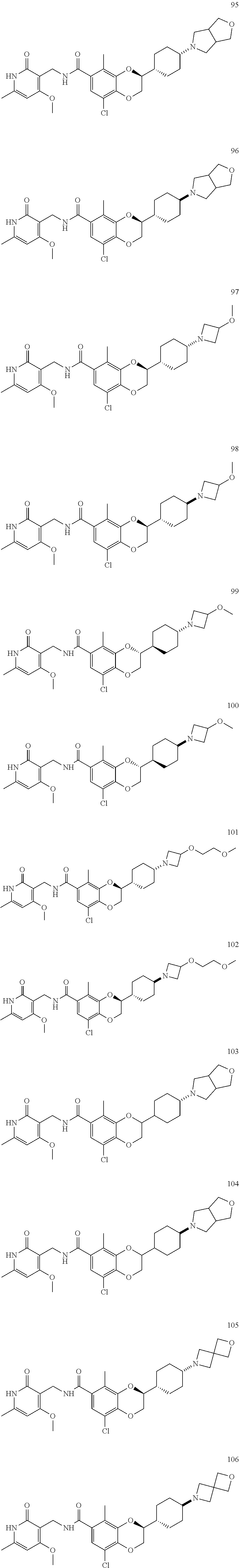

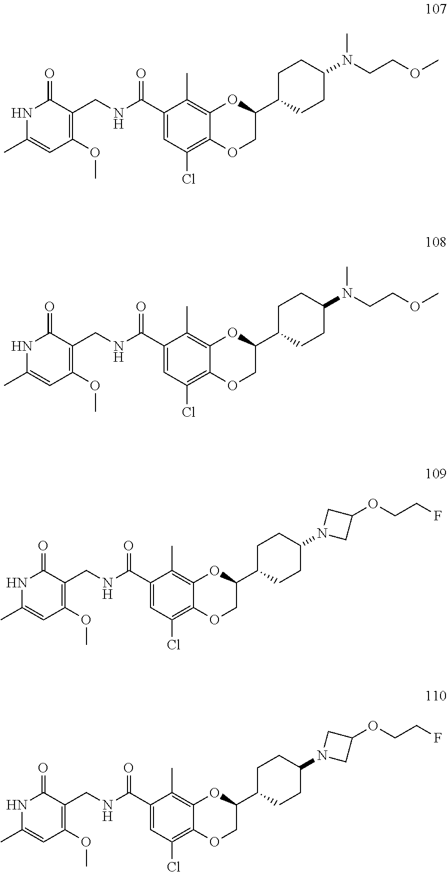

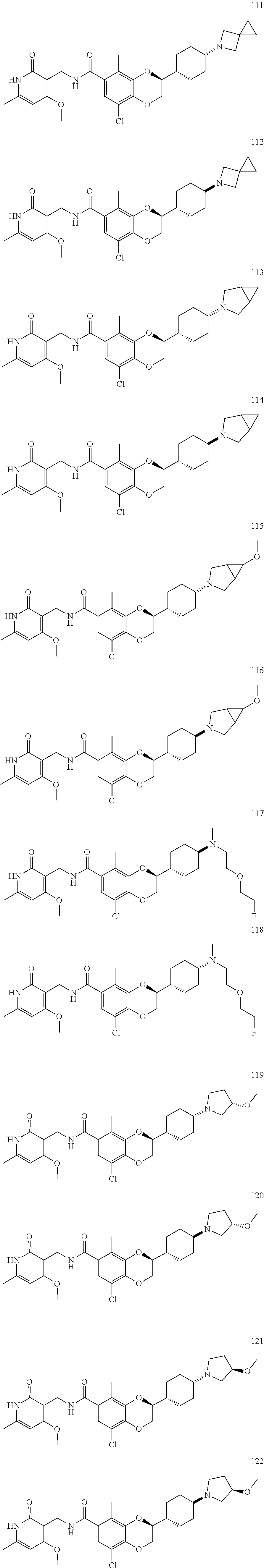

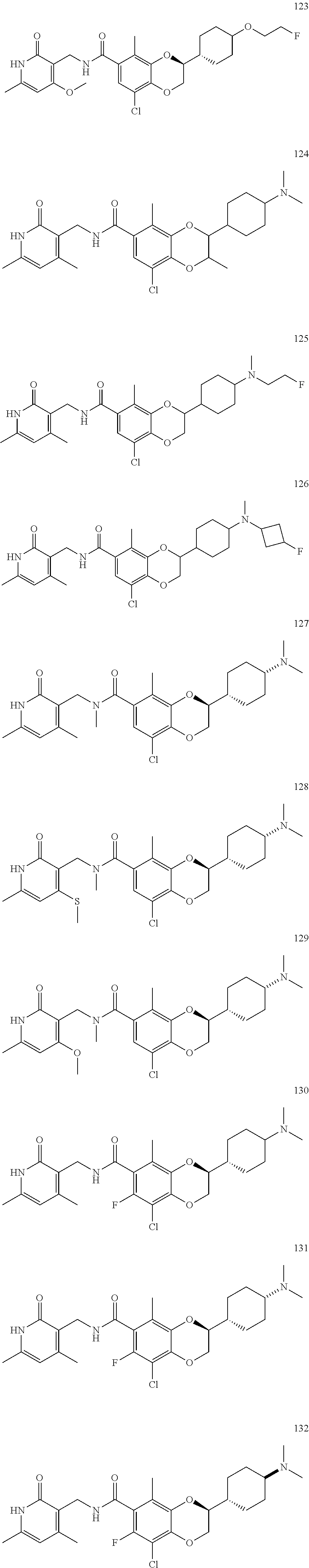

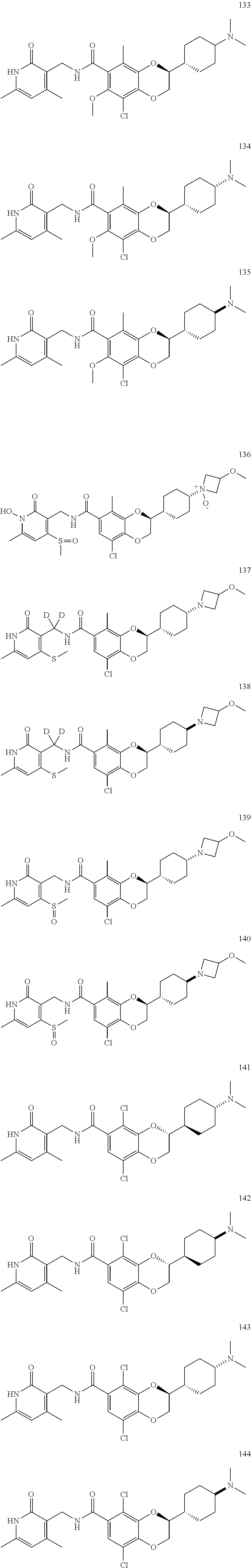

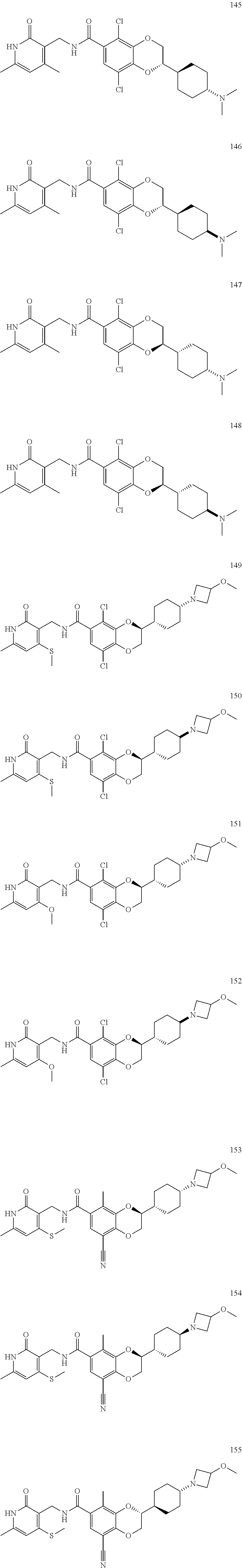

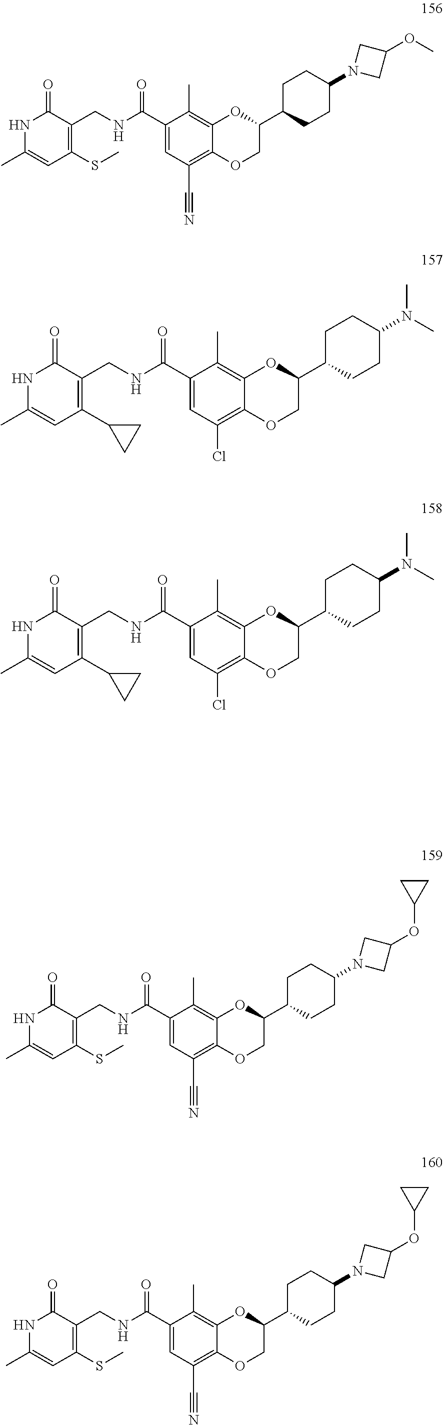

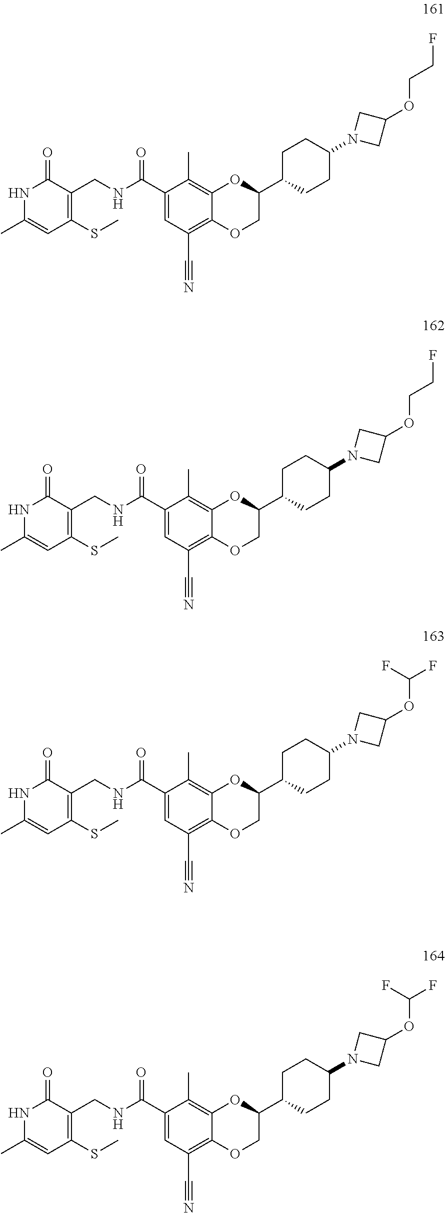 [SMILES: CSc1cc(C)[nH]c(=O)c1CNC(=O)c1cc(C#N)c2c(c1C)O[C@@H]([C@H]1CC[C@@H](N3CC(OCCF)C3)CC1)CO2 ; CSc1cc(C)[nH]c(=O)c1CNC(=O)c1cc(C#N)c2c(c1C)O[C@@H]([C@H]1CC[C@H](N3CC(OCCF)C3)CC1)CO2 ; CSc1cc(C)[nH]c(=O)c1CNC(=O)c1cc(C#N)c2c(c1C)O[C@@H]([C@H]1CC[C@@H](N3CC(OC(F)F)C3)CC1)CO2 ; CSc1cc(C)[nH]c(=O)c1CNC(=O)c1cc(C#N)c2c(c1C)O[C@@H]([C@H]1CC[C@H](N3CC(OC(F)F)C3)CC1)CO2]

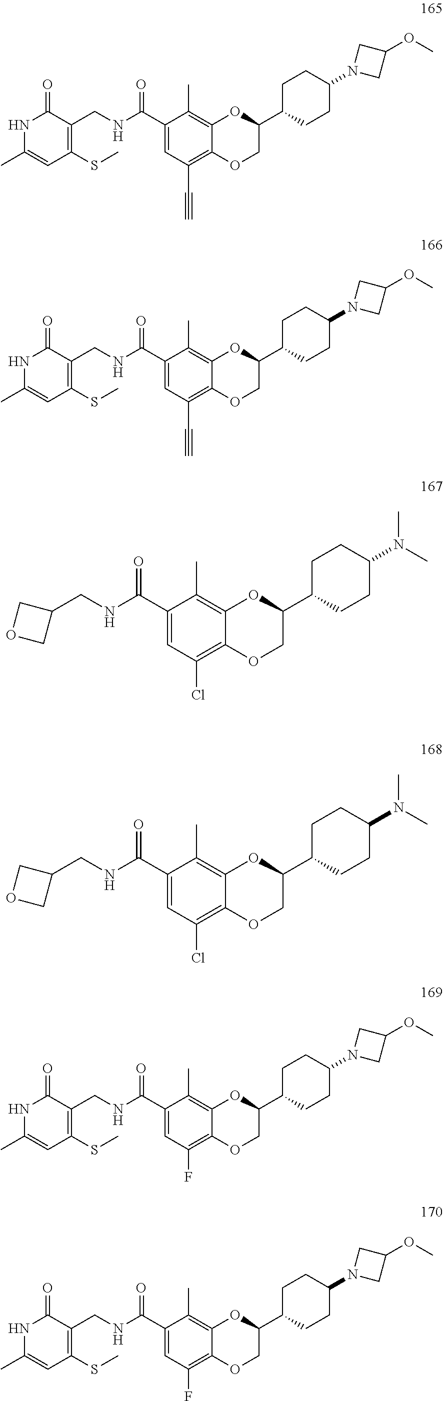

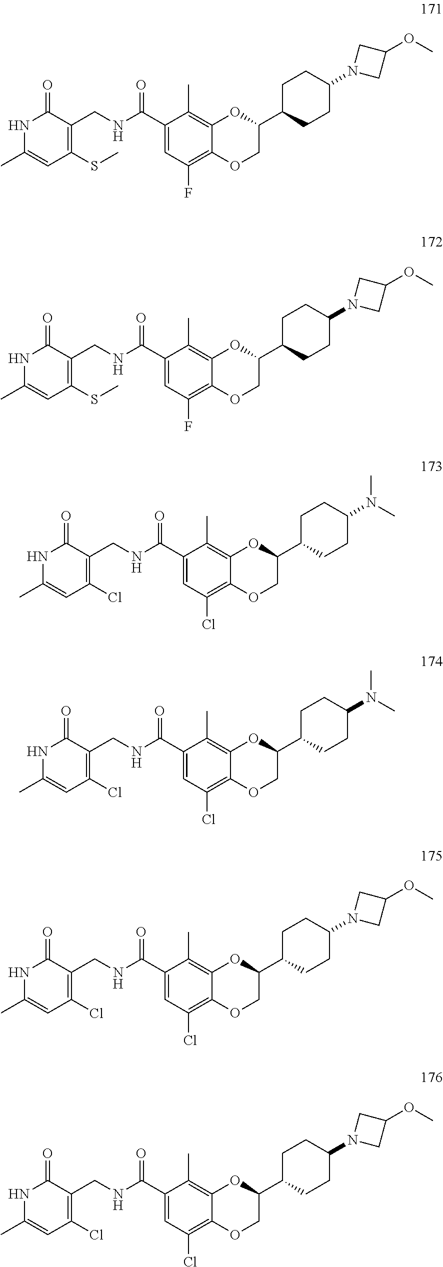

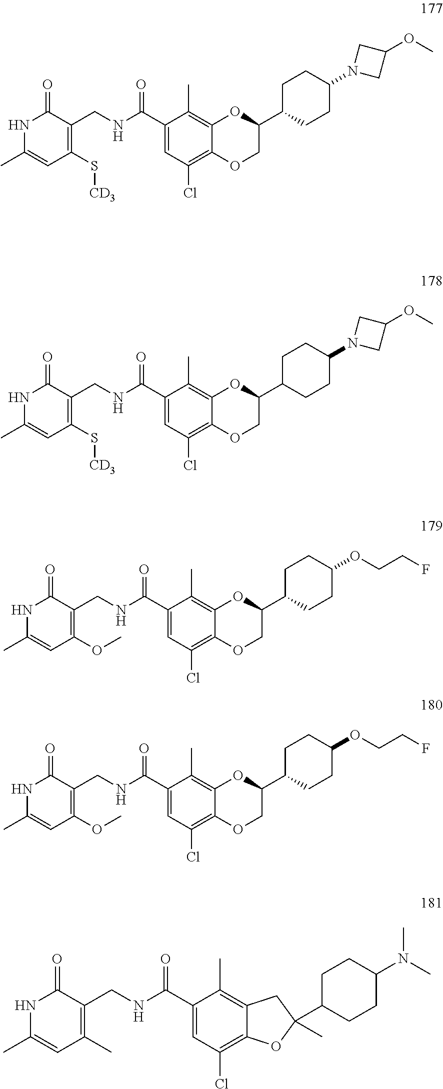

In the embodiment of the present invention, the compound of the present invention, or a deuterated compound thereof, or a stereoisomer thereof, or a pharmaceutically acceptable salt thereof, is characterized in that, the compound represented by Formula I or Formula II is specifically:

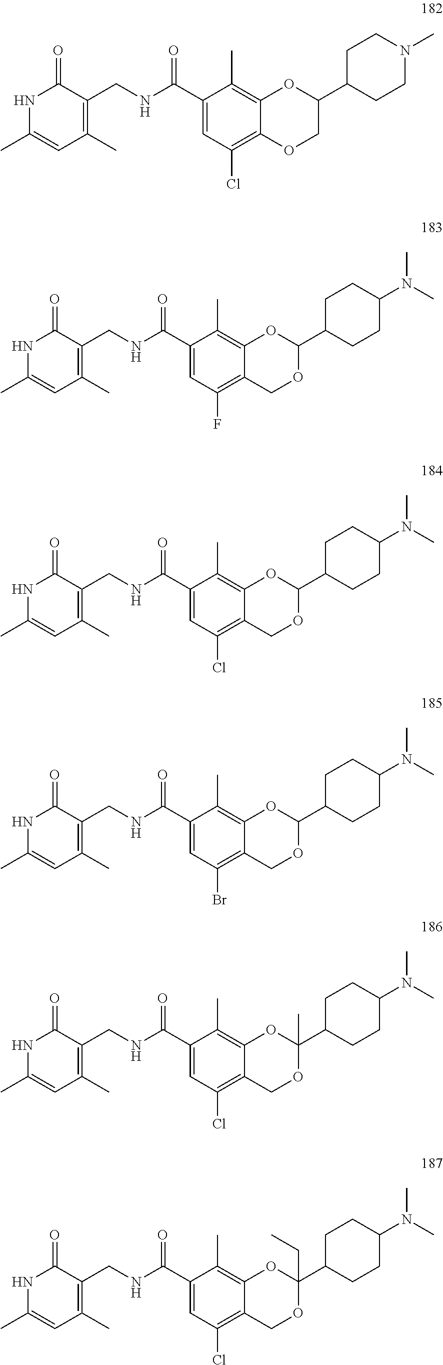

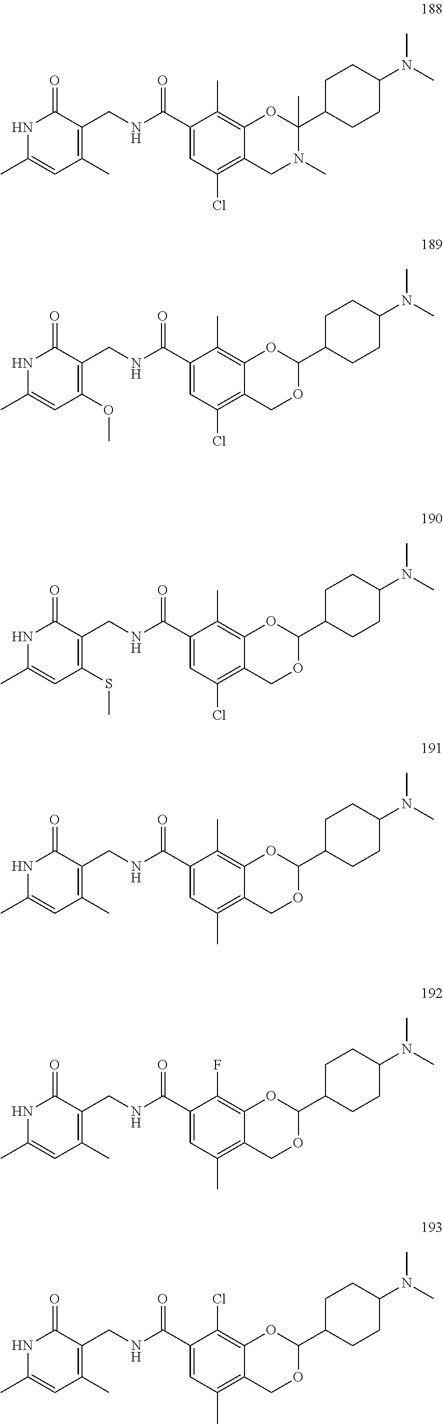

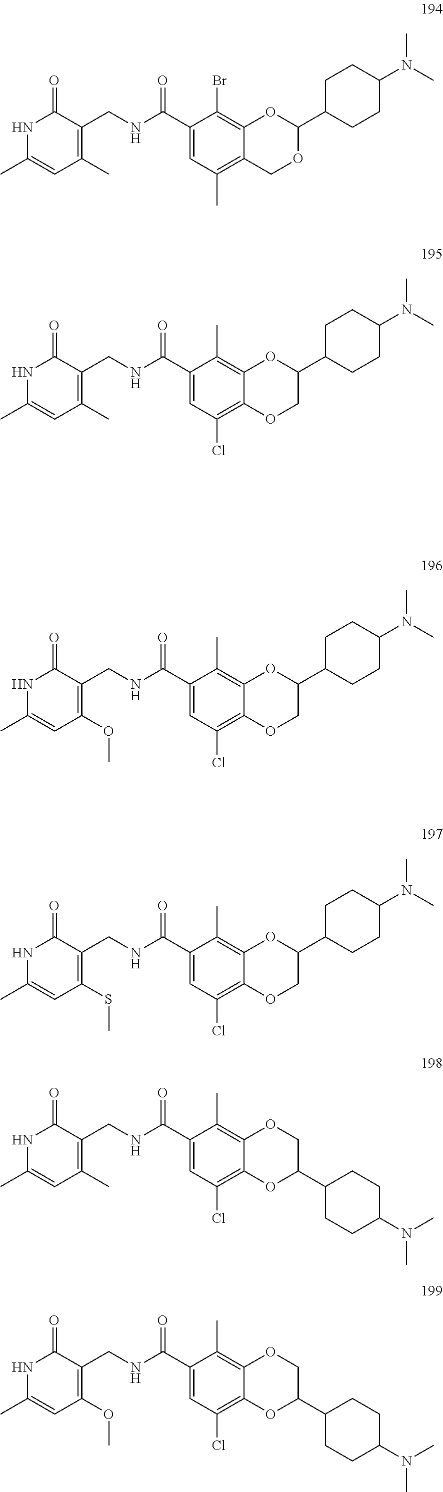

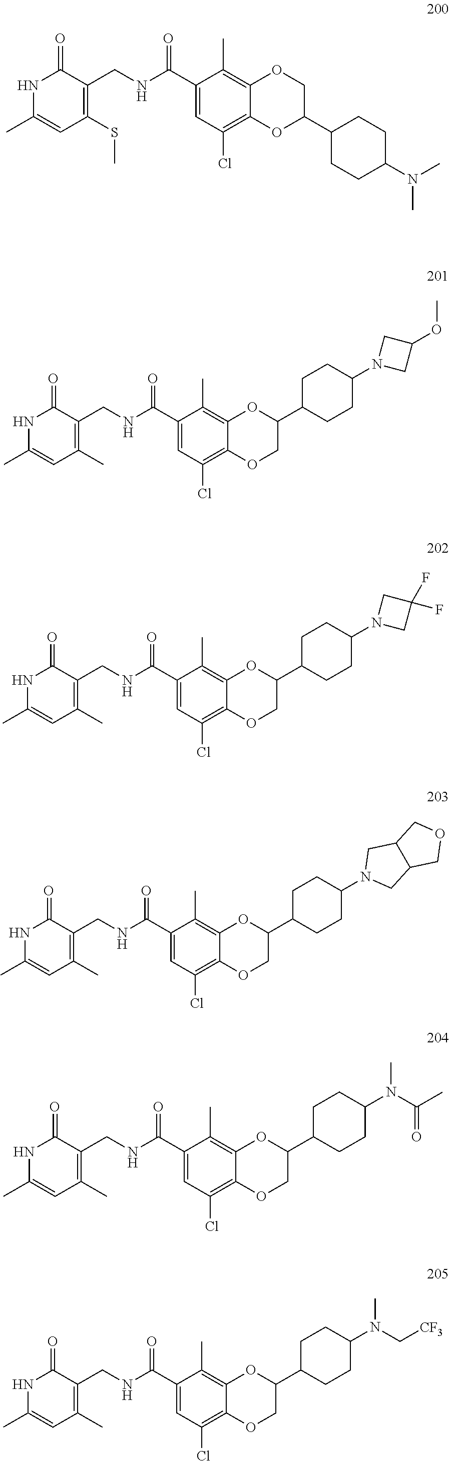

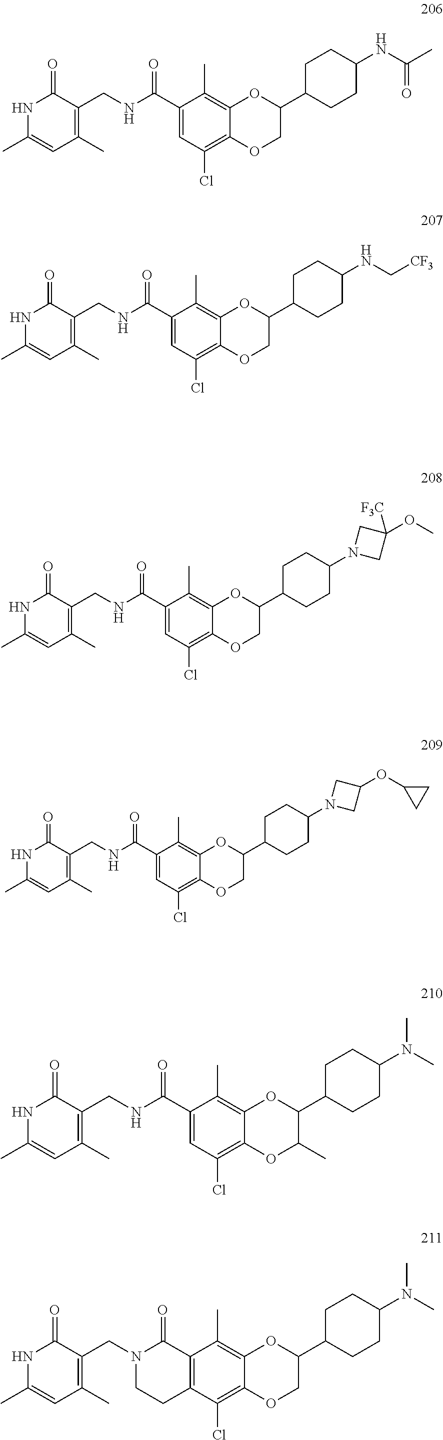

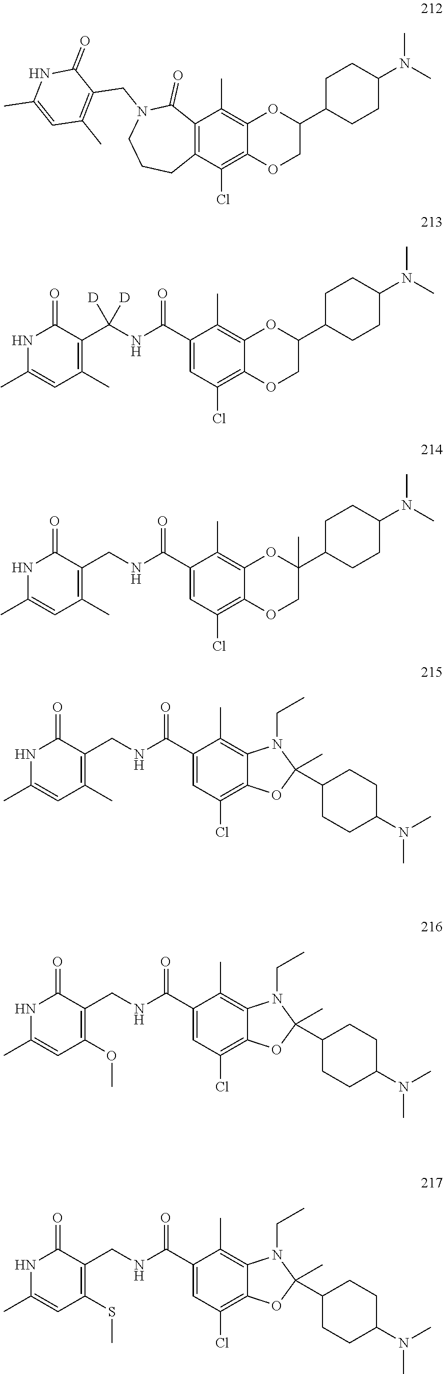

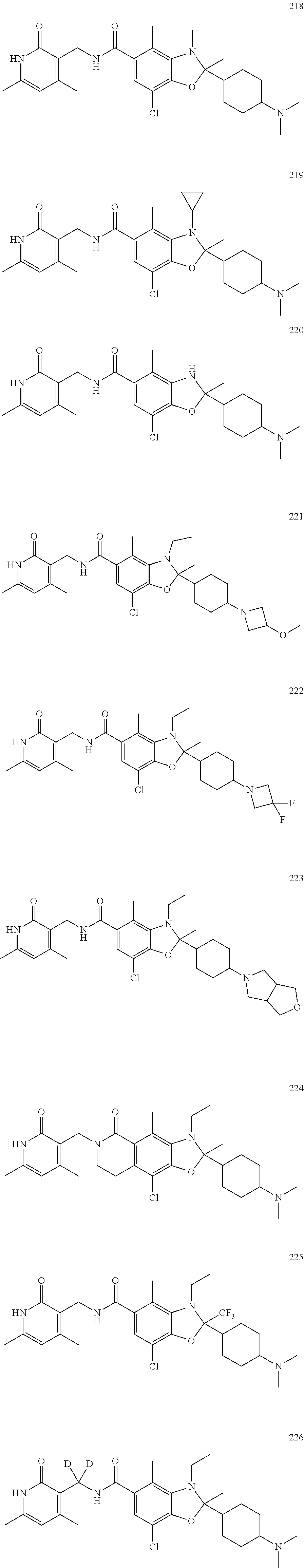

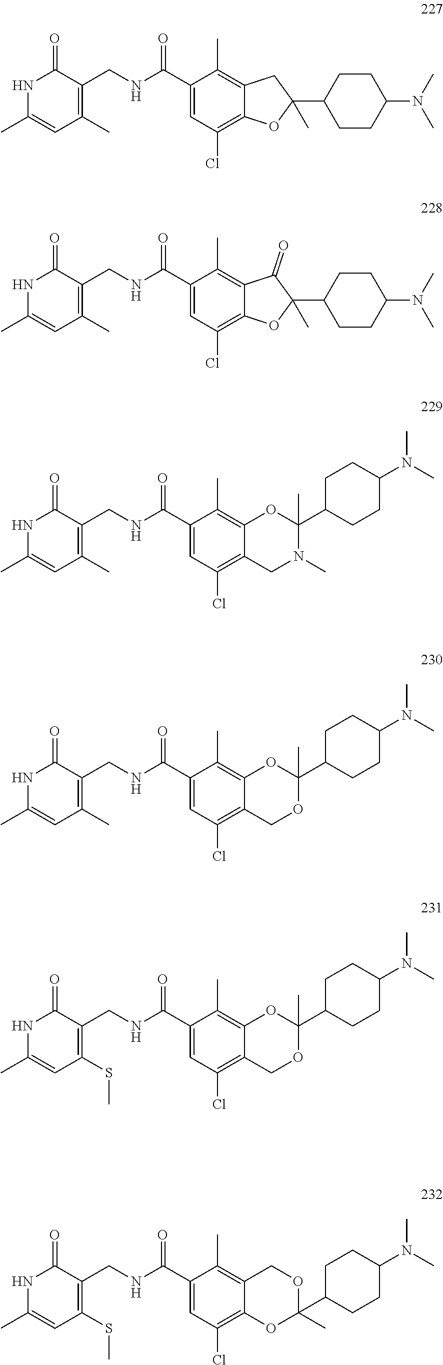

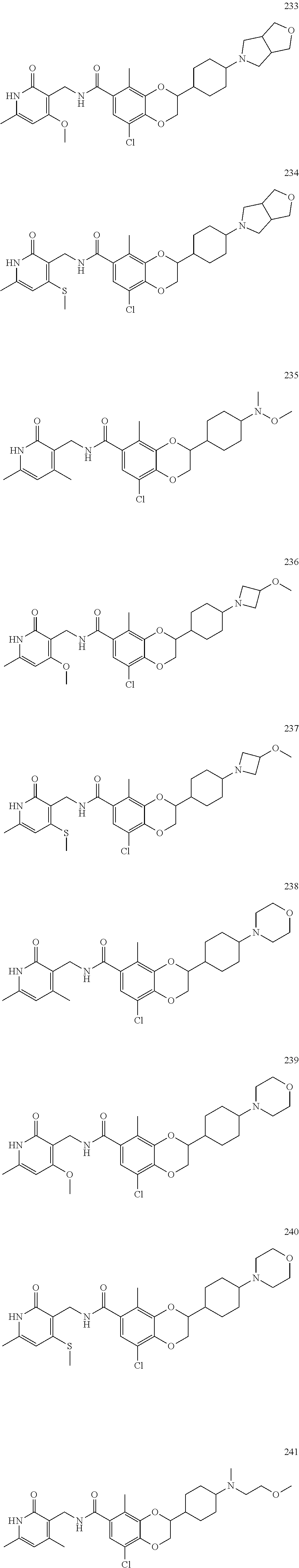

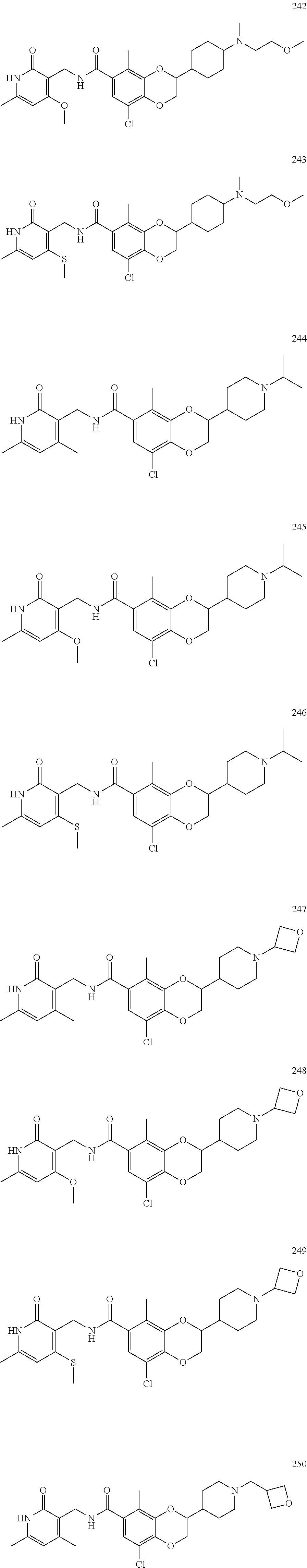

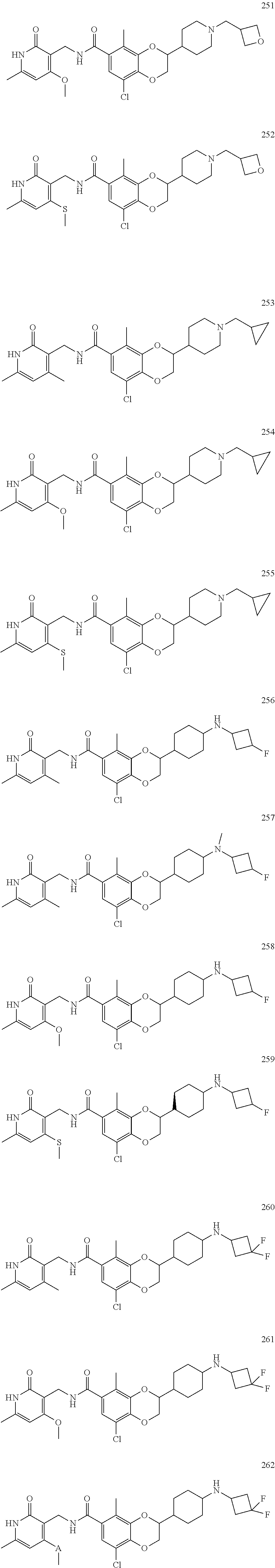

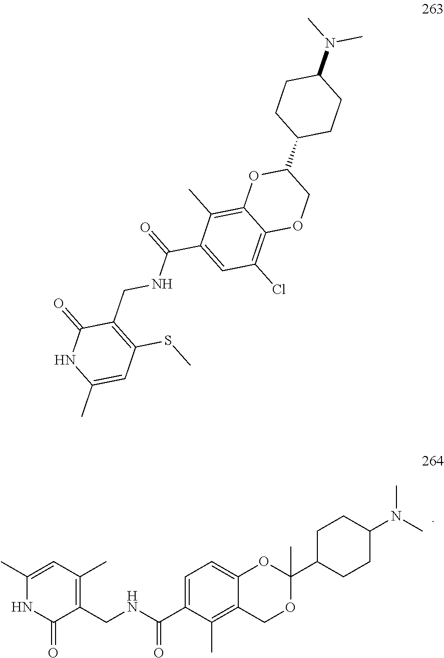

The present invention further an intermediate compound shown as follows:

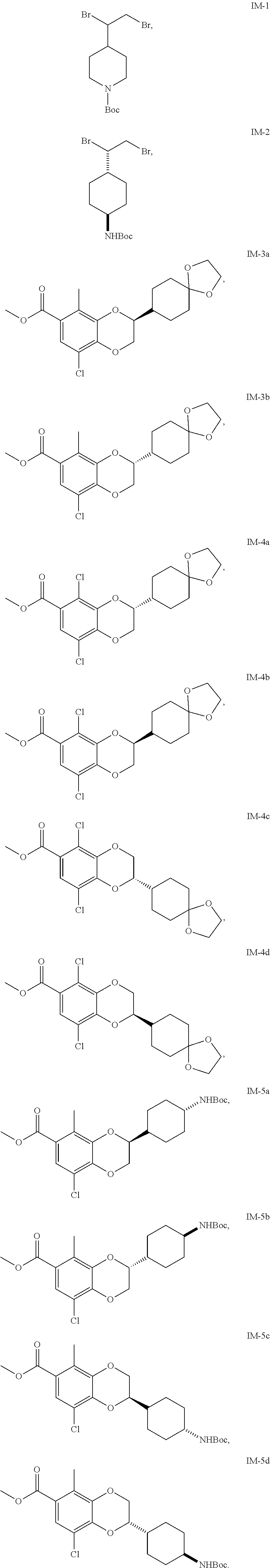

The present invention further provides the use of the compound of the present invention, or a stereoisomer thereof, or a pharmaceutically acceptable salt thereof in the preparation of a medicament for treating an EZH1/2-mediated disease.

Further, the EZH1/2-mediated disease is one or more selected from diseases related to inflammation, autoimmune disease, infectious disease, cancer, and precancerous syndrome.

The present invention further provides a pharmaceutical composition, comprising the compound of the present invention, or a stereoisomer thereof, or a pharmaceutically acceptable salt thereof as an active pharmaceutical ingredient, and if necessary, a pharmaceutically acceptable excipient to prepare a formulation.

The present invention further provides a preparation method of the compound of the present invention, or a deuterated compound thereof, or a stereoisomer thereof, or a pharmaceutically acceptable salt thereof.

In an embodiment of the present invention, the preparation method of the compound of Formula I

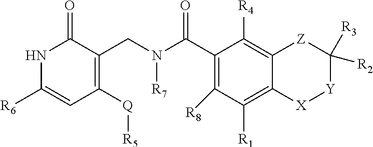

is as follows:

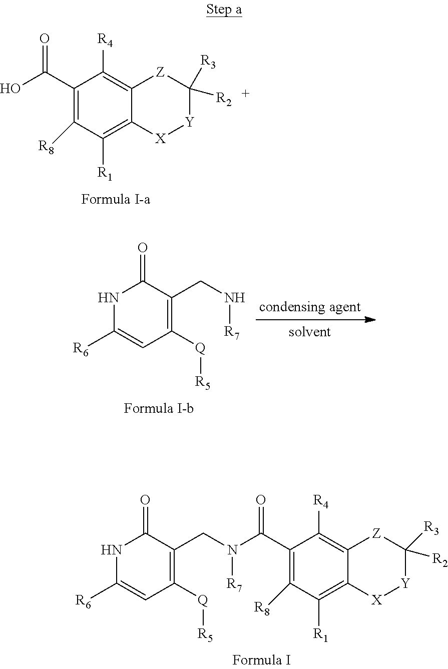

The compounds of Formula I-a and Formula I-b are subjected to amide condensation reaction under the condition of condensing agent, to prepare the compound of Formula I. The compound of Formula I-b is commercially available or can be synthesized by general organic synthesis method;

### Step b

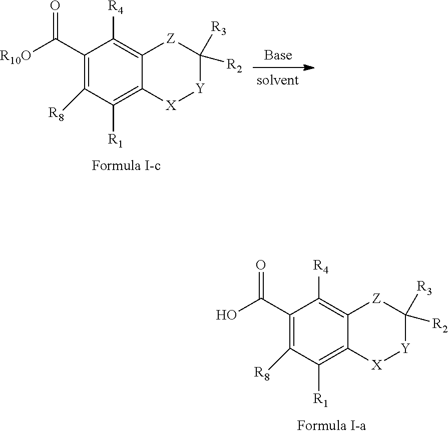

The compound of Formula I-c is subjected to ester hydrolysis reaction under basic condition to give the compound of Formula I-a, wherein the substituent R₁₀ is C₁-C₆alkyl group, for example, methyl, ethyl, propyl (n-propyl and isopropyl), butyl (n-butyl, isobutyl and t-butyl), pentyl (n-pentyl, isopentyl and neopentyl) and hexyl.

### Step c

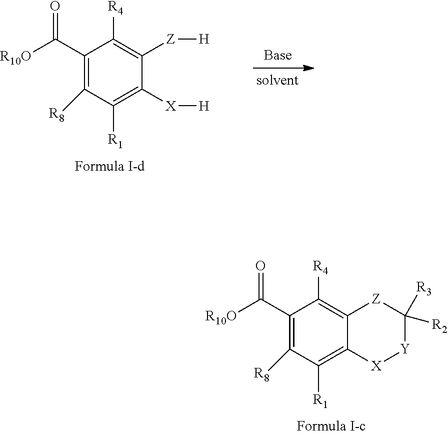

Formula I-d is subjected to cyclization reaction under basic condition, to prepare the compound of Formula I-c.

In the above Steps a-c, the substituents R₁, R₂, R₃, R₄, R₅, R₆, R₇, R₈ in each intermediate compound of Formula I-a, Formula I-b, I-c and I-d have the same definition with the definition above.

In Steps a-c, said condensing agent includes but is not limited to O-(7-azabenzotriazole-1-yl)-bis(dimethylamino)carbonium hexafluorophosphate (HATU), O-(benzotriazole-1-yl)-bis(dimethylamino)carbonium hexafluorophosphate (HBTU), 0-(5-chlorobenzotriazole-1-yl)-bis(dimethylamino)carbonium hexafluorophosphate (HCTU), O-(benzotriazole-1-yl)-bis(dimethylamino)carbonium tetrafluoroborate (TBTU), O—(N-succinimidyl)-bis(dimethylamino)carbonium tetrafluoroborate (TSTU), O—(N-endo-5-norbornene-2,3-dicarboxylimide)-bis(dimethylamino)carbonium tetrafluoroborate (TNTU), benzotriazole-1-yloxy-tris(tetrahydropyrrolyl)phosphonium hexafluorophosphate (PyBOP) and the like; wherein said base is one or two or more selected from a group consisting of organic base or inorganic base, inorganic base is one or two or more selected from a group consisting of sodium hydride, sodium carbonate, potassium carbonate, cesium carbonate, sodium bicarbonate, potassium bicarbonate, sodium hydroxide, potassium hydroxide, lithium hydroxide or barium hydroxide; organic base is one or two or more selected from a group consisting of N,N-diisopropylethylamine, triethylamine, lithium diisopropylamide, n-(C₁₋₆alkyl)lithium, lithium hexamethyldisilazane, potassium hexamethyldisilazane, sodium hexamethyldisilazane, lithium tetramethylpiperidine, potassium butoxide, potassium pentoxide, potassium pentanolate. Among others, said solvent is one or two or more of dichloromethane, chloroform, ethyl acetate, n-hexane, methyl t-butyl ether, ethyl ether, DMF, water, methanol, ethanol, propanol, butanol.

In an embodiment of the present invention, the preparation method of Compound II

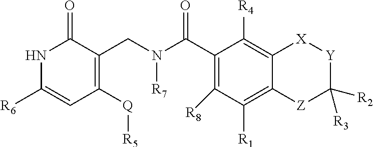

is as follows.

### Step a

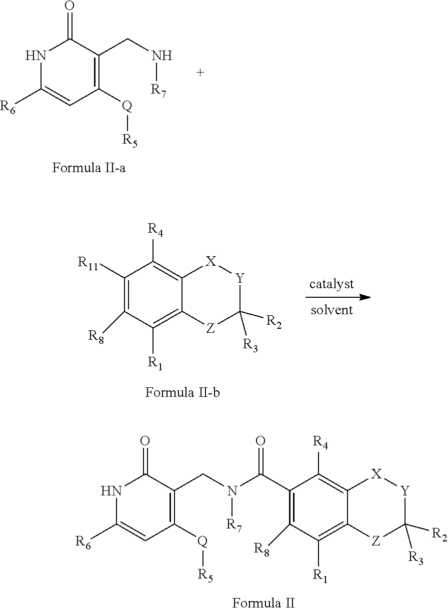

The compounds of Formula II-a and Formula II-b are subjected to coupling reaction via insertion of carbonyl group under catalyst and solvent condition, to prepare the compound of Formula II. The compound of Formula II-a is commercially available or can be synthesized by general organic synthesis method. Among others, the catalyst is molybdenum hexacarbonyl, Pd(dppf)Cl2.DCM, K2CO3; the solvent is one or more or two of dichloromethane, chloroform, ethyl acetate, n-hexane, methyl t-butyl ether, ethyl ether, DMF, water, methanol, ethanol, propanol, butanol, toluene, 1,4-dioxane; and the substituent Rn is halogen group, including F, Cl, Br or I.

### Step b

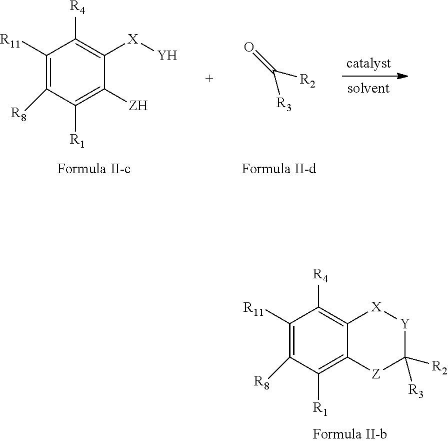

The compounds of Formula II-c and Formula II-d are subjected to cyclization reaction under catalyst and solvent condition, to prepare the compound of Formula II-b. The compound of Formula II-c is commercially available or can be synthesized by general organic synthesis method. Among others, in the cyclization step, the catalyst is selected from a catalyst to catalyze the acetal formation reaction of alcohol and aldehyde, including but not limited to one or two or more of PTSA (p-tosylic acid), PPTS (pyridium p-tosylate)+molecular sieve, Bronsted acid, hydrochloric acid, and sulfuric acid. The solvent is one or two or more of dichloromethane, chloroform, ethyl acetate, n-hexane, methyl t-butyl ether, ethyl ether, DMF, water, methanol, ethanol, propanol, butanol, toluene, 1,4-dioxane.

In the above Steps a-b, the substituents R₁, R₂, R₃, R₄, R₅, R₆, R₇, R₈ in each intermediate compound of Formula II-a, Formula II-b, II-c and II-d have the same definition with the definition above.

In an embodiment of the present invention, the preparation method of Compound IIb

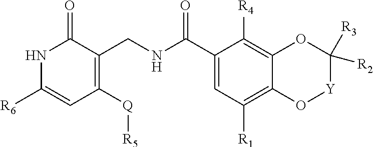

is as follows.

### Step a

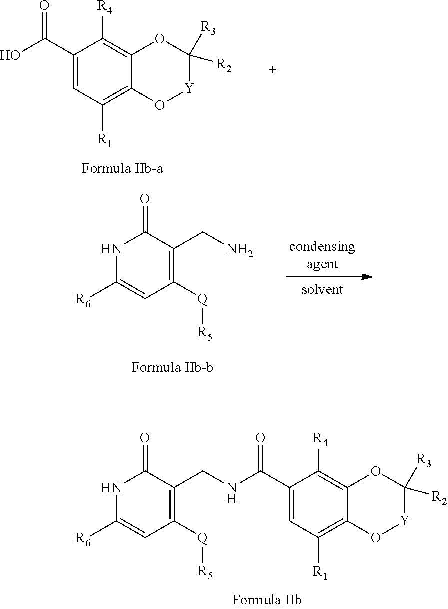

The compounds of Formula IIb-a and Formula IIb-b are subject to amide condensation reaction under the condition of condensing agent, to prepare the compound of Formula IIb. The compound of Formula IIb-b is commercially available or can be synthesized by general organic synthesis method.

### Step b

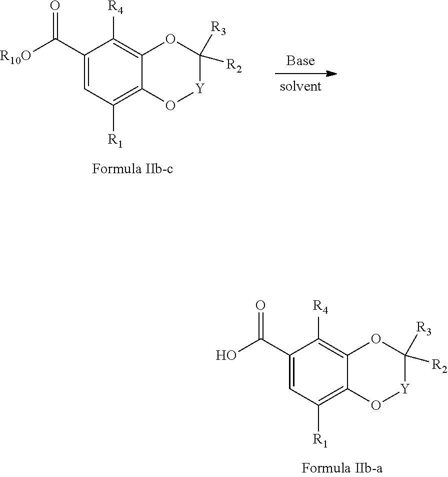

The compound of Formula IIb-c is subjected to ester hydrolysis reaction under basic condition to give the compound of Formula IIb-a, wherein the substituent R₁₀ is C₁-C₆alkyl group, for example, methyl, ethyl, propyl (n-propyl and isopropyl), butyl (n-butyl, isobutyl and t-butyl), pentyl (n-pentyl, isopentyl and neopentyl) and hexyl.

### Step c

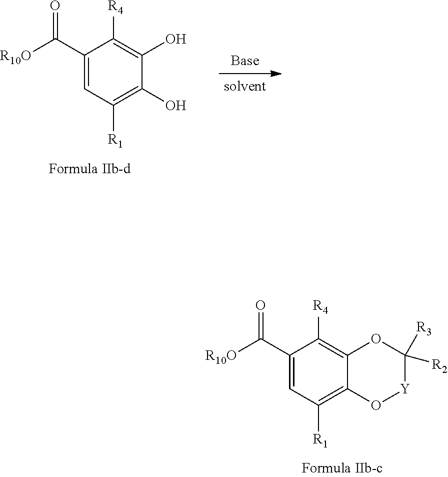

Formula IIb-d is subjected to cyclization reaction under basic condition, to prepare the compound of Formula IIb-c.

In the above Steps a-c, the substituents R₁, R₂, R₃, R₄, R₅, R₆, R₇, R₈ in each intermediate compound of Formula IIb-a, Formula IIb-b, IIb-c and IIb-d have the same definition with the definition above.

In Steps a-c, the condensing agent includes but is not limited to O-(7-azabenzotriazole-1-yl)-bis(dimethylamino)carbonium hexafluorophosphate (HATU), O-(benzotriazole-1-yl)-bis(dimethylamino)carbonium hexafluorophosphate (HBTU), O-(5-chlorobenzotriazole-1-yl)-bis(dimethylamino)carbonium hexafluorophosphate (HCTU), O-(benzotriazole-1-yl)-bis(dimethylamino)carbonium tetrafluoroborate (TBTU), O—(N-succinimidyl)-bis(dimethylamino)carbonium tetrafluoroborate (TSTU), O—(N-endo-5-norbornene-2,3-dicarboxylimide)-bis(dimethylamino)carbonium tetrafluoroborate (TNTU), benzotriazole-1-yloxy-tris(tetrahydropyrrolyl)phosphonium hexafluorophosphate (PyBOP) and the like. Among others, the base is selected from a group consisting of organic base or inorganic base, and the inorganic base is one or two or more selected from a group consisting of sodium hydride, sodium carbonate, potassium carbonate, cesium carbonate, sodium bicarbonate, potassium bicarbonate, sodium hydroxide, potassium hydroxide, lithium hydroxide or barium hydroxide; and the organic base is one or two or more selected from a group consisting of N,N-diisopropylethylamine, triethylamine, lithium diisopropylamide, n-(C₁₋₆alkyl)lithium, lithium hexamethyldisilazane, potassium hexamethyldisilazane, sodium hexamethyldisilazane, lithium tetramethylpiperidine, potassium butoxide, potassium pentoxide, potassium pentanolate. The solvent is one or two or more of dichloromethane, chloroform, ethyl acetate, n-hexane, methyl t-butyl ether, ethyl ether, DMF, water, methanol, ethanol, propanol, butanol.

In another two embodiments of the present invention, the preparation methods of Compound IIc

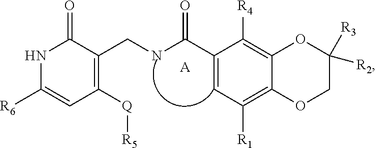

### Compound IIa

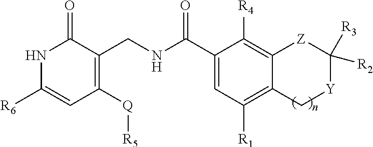

are similar to the preparation methods of Compound I and Compound IIb.

The followings are the explanation and definition to the terms used in the present invention.

“Cancer” or “malignant tumor” means any of a variety of diseases characterized by uncontrolled abnormal proliferation of cells, the ability of affected cells to spread to other sites locally or through the bloodstream and lymphatic system body (i.e. metastases) and any of a number of characteristic structural and/or molecular features. “Cancer cell” refers to cells that undergo early, intermediate or late stages of multi-step tumor progression. Cancers include sarcomas, breast cancer, lung cancer, brain cancer, bone cancer, liver cancer, kidney cancer, colon cancer and prostate cancer. In some embodiments, the compound of Formula I or Formula II is used to treat a cancer selected from colon cancer, brain cancer, breast cancer, fibrosarcoma, and squamous cell carcinoma. In some embodiments, the cancer is selected from melanoma, breast cancer, colon cancer, lung cancer, and ovarian cancer. In some embodiments, the cancer treated is a metastatic cancer.

Autoimmune diseases are caused by the body's immune response to substances and tissues normally present in the body. Examples of autoimmune diseases include myocarditis, lupus nephritis, primary biliary cirrhosis, psoriasis, type 1 diabetes, Graves' disease, celiac disease, Crohn's disease, autoimmune neutropenia, juvenile arthritis, rheumatoid arthritis, fibromyalgia, Guillain-Barre syndrome, multiple sclerosis, and autoimmune retinopathy. Some embodiments of the invention relate to the treatment of autoimmune diseases such as psoriasis or multiple sclerosis.

Inflammatory diseases include a variety of conditions characterized by histopathic inflammation. Examples of inflammatory diseases include acne vulgaris, asthma, celiac disease, chronic prostatitis, glomerulonephritis, inflammatory bowel disease, pelvic inflammatory disease, reperfusion injury, rheumatoid arthritis, sarcoidosis, vasculitis, airway inflammation and interstitial cystitis caused by house dust mites. Significant overlap exists between inflammatory and autoimmune diseases. Some embodiments of the invention relate to the treatment of the inflammatory disease asthma. The immune system is often involved in inflammatory diseases, as seen in anaphylaxis and some myopathies, and many immune system disorders result in abnormal inflammation. EZH1/2-mediated diseases also include autoimmune inflammatory diseases.

The compounds and derivatives provided in the present invention may be named according to the IUPAC (International Union of Pure and Applied Chemistry) or CAS (Chemical Abstracts Service, Columbus, OH) nomenclature system.

Definition of terms used in the present invention: Unless otherwise stated, the initial definition provided by a group or term herein applies to the group or term throughout the specification. For terms that are not specifically defined herein, they should be interpreted based on the disclosure and context, with the meanings which a person skilled in the art can assign to them.

“Substitution” means that a hydrogen atom in a molecule is replaced by a different atom or molecule.

The minimum and maximum carbon atom content in a hydrocarbon group is indicated by a prefix, for example, the prefix C_{a-b} alkyl indicates any alkyl group containing “a” to “b” carbon atoms. Thus, for example, “C₁₋₄ alkyl” refers to an alkyl group containing 1 to 4 carbon atoms.

“Alkyl” means a saturated hydrocarbon chain having the indicated number of member atoms. For example, C₁₋₆ alkyl refers to an alkyl group having 1 to 6 member atoms, for example, 1 to 4 member atoms. Alkyl groups can be straight or branched. Representative branched alkyl groups have one, two or three branches. Alkyl groups may be optionally substituted with one or more substituents as defined herein. Alkyl groups include methyl, ethyl, propyl (n-propyl and isopropyl), butyl (n-butyl, isobutyl and t-butyl), pentyl (n-pentyl, isopentyl and neopentyl) and hexyl. Alkyl groups may also be part of other groups such as C₁˜C₆ alkoxy groups.

“Cycloalkyl” and “cycloalkane” means a saturated or partially saturated cyclic group having carbon atoms and no ring heteroatoms, and having a single ring or multiple rings (including fused and combined). For polycyclic ring systems having aromatic and non-aromatic rings containing no ring heteroatoms, the term “cycloalkyl” is applied when the binding site is on the non-aromatic carbon (for example. 5,6,7,8-tetralin-5-yl). The term “cycloalkyl” includes cycloalkenyl groups such as cyclohexenyl. Examples of cycloalkyl groups include, for example, adamantyl, cyclopropyl, cyclobutyl, cyclohexyl, cyclopentyl, cyclooctyl, cyclopentenyl, and cyclohexenyl. Examples of cycloalkyl groups including multiple bicycloalkyl ring systems are bicyclohexyl, bicyclopentyl, bicyclooctyl and the like, for example

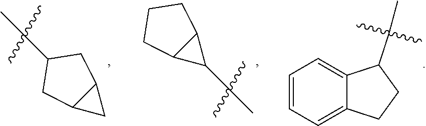 [SMILES: *C1CC2CC2C1 ; *C1C2CCCC12 ; *C1CCc2ccccc21]

“Alkenyl” means a straight-chain or branched-chain hydrocarbon group having 2 to 10 carbon atoms and in some embodiments 2 to 6 carbon atoms or 2 to 4 carbon atoms, and having at least one ethylenic unsaturated site (>C═CC<). For example, (Cₐ-C_{b})alkenyl refers to an alkenyl group having a to b carbon atoms and is intended to include, for example, ethenyl, propenyl, isopropenyl, 1,3-butadienyl, and the like.

“Alkynyl” means a straight-chain or branched-chain monovalent hydrocarbon group containing at least one triple bond. The term “alkynyl” is also intended to include those hydrocarbyl groups having one triple bond and one double bond. For example, (C₂-C₆)alkynyl is intended to include ethynyl, propynyl, and the like.

“Halogen” is fluorine, chlorine, bromine or iodine.

“Haloalkyl” means that the hydrogen atoms in the alkyl group may be replaced by one or more halogen atoms. For example, C_{1˜4} haloalkyl refers to an alkyl group containing 1-4 carbon atoms whose hydrogen atoms are replaced by one or more halogen atoms.

“Heterocyclic ring”, “heterocycloalkyl”, and “heterocycloalkane” refer to a saturated ring or a non-aromatic unsaturated ring containing at least one heteroatom; wherein the heteroatom refers to nitrogen atom, oxygen atom, or sulfur atom;

“Heteroaryl ring” refers to an aromatic unsaturated ring containing at least one heteroatom; wherein the heteroatom refers to nitrogen atom, oxygen atom, sulfur atom;

The compounds of the present invention may also contain unnatural proportions of atomic isotopes such as deuterium (²H), tritium (³H), for example, at one or more atoms constituting the compound. Deuterated compounds generally retain comparable activity to non-deuterated compounds, and can achieve better metabolic stability when deuterated at certain sites, resulting in certain therapeutic advantages, such as increased in vivo half-life or dosage reduced demand and the like. Therefore, in the present invention, the number of deuterium atoms in the deuterated substance can be 1, 2, 3, 4, 5, 6, 7 or more;

“Stereoisomer” includes enantiomers and diastereomers. The compound of the present invention may contain asymmetric centers or chiral centers and thus exist as different stereoisomers. All stereoisomeric forms of the compounds of the present invention, including but not limited to, diastereomers, enantiomers, atropisomers, and mixtures thereof, such as racemic mixtures, constitute a part of the present invention. Many organic compounds exist in optically active forms, that is, they have the ability to rotate the plane of plane-polarized light. When describing optically active compounds, the prefixes D, L or R, S are used to indicate the absolute configuration of the molecular chiral center. The chemical structures of these stereoisomers are the same, but their three-dimensional structures are different. A particular stereoisomer may be an enantiomer, and a mixture of isomers is often referred to as an enantiomeric mixture. A 50:50 mixture of enantiomers is known as a racemic mixture or racemate, which can result in no stereoselectivity or stereospecificity during a chemical reaction. The terms “racemic mixture” and “racemate” refer to an equimolar mixture of two enantiomers, devoid of optical activity.

The term “pharmaceutically acceptable” means that a certain carrier, vector, diluent, excipient, and/or formed salt are generally chemically or physically compatible with other ingredients that constitute a pharmaceutical dosage form, and are physiologically compatible with receptors.

The pharmaceutical composition of the present invention can be in any form of reusable pharmaceutical preparations, such as: oral, injection, external preparations, and the like. The oral dosage forms include but not limited to: tablets, capsules, oral liquids, granules, pills, suspensions, and injections are selected from water injections and powder injections, and external preparations are selected from patches and ointments. All preparations can be prepared according to conventional techniques of pharmacy, such as using any one of the compound of the present invention, or a stereoisomer thereof, or a pharmaceutically acceptable salt thereof as a pharmaceutically active ingredient, and adding pharmaceutically acceptable carrier to prepare into the above-mentioned pharmaceutical dosage form suitable for taking, wherein the unit dose of the active ingredient can be 0.1 mg-1000 mg, such as each tablet contains 0.1 mg-1000 mg, preferably 5-500 mg of the active ingredient.

The terms “salt” and “pharmaceutically acceptable salt” refer to an acidic and/or basic salt formed with the above-mentioned compound or a stereoisomer thereof and inorganic and/or organic acids and bases, and also include zwitterionic salts (internal salts), also include quaternary ammonium salts, such as alkyl ammonium salts. These salts may be obtained directly in the final isolation and purification of the compounds. It can also be obtained by mixing the above-mentioned compound, or a stereoisomer thereof, with a certain amount of acid or base as appropriate (for example, equivalent). These salts may form precipitates in solution and be collected by filtration, or may be recovered after evaporation of the solvent, or may be obtained by freeze-drying after reaction in an aqueous medium. Said salt in the present invention can be hydrochloride, sulfate, citrate, benzene sulfonate, hydrobromide, hydrofluoride, phosphate, acetate, propionate, dibutyl salt, oxalate, malate, succinate, fumarate, maleate, tartrate, or trifluoroacetate.

In certain embodiments, one or more compounds of the invention may be used in combination with each other. Alternatively, the compound of the present invention may be used in combination with any other active agents for the preparation of drugs or pharmaceutical compositions for regulating cell functions or treating diseases. If a group of compounds is used, the compounds may be administered to the subject simultaneously, separately or sequentially.

Apparently, according to the above content of the present invention, according to common technical knowledge and conventional means in this field, without departing from the above basic technical idea of the present invention, other various forms of modification, replacement or change can also be made.

The beneficial effects of the present invention are:

The EZH1/2 inhibitor of the present invention is superior to existing EZH2 inhibitors in both in vivo and in vitro activities. The above-mentioned content of the present invention will be further described in detail below through specific implementation in the form of examples. However, this should not be construed as limiting the scope of the above-mentioned subject matter of the present invention to the following examples. All technologies realized based on the above contents of the present invention belong to the scope of the present invention.

### DESCRIPTION OF DRAWINGS

FIG. 1 is a graph showing the inhibition of compound DS-3201 on tumor growth in mice;

FIG. 2 is a graph showing the inhibition of Compound 8 on tumor growth in mice;

FIG. 3 is a graph showing the inhibition of Compound 97 on tumor growth in mice;

FIG. 4 is the inhibition of Compound 97 and 169 on tumor growth in mice;

### DETAILED DESCRIPTION OF THE INVENTION

The structure of the compound was determined by nuclear magnetic resonance (NMR) and mass spectrometry (MS). The NMR shift (δ) was given in units of 10⁻⁶ (ppm). The NMR was measured by (Bruker Avance III 400 and Bruker Avance 300) nuclear magnetic apparatus.

Deuterated methyl sulfoxide (DMSO-d₆), deuterated chloroform (CDCl₃) and deuterated methanol (CD₃OD) were used as the characterization solvents, and tetramethylsilane (TMS) was used as the internal standard.

The LC-MS was determined by Shimadzu LC-MS 2020 (ESI). The HPLC was determined by Shimadzu LC-20A. MPLC (medium performance liquid chromatography) was conducted by Gilson GX-281 reverse phase preparative chromatography. Yantai Huanghai HSGF254 or Qingdao GF254 silica gel plate was used as the silica gel plate for thin-layer chromatography, and the specification of thin-layer chromatography separation and purification products was 0.4 mm-0.5 mm. Column chromatography generally used Yantai Huanghai silica gel 200-300 mesh silica gel as carrier.

The known starting materials of the present invention can be synthesized by or according to the methods known in the field, or can be purchased from Anneiji Chemical, Chengdu Kelon Chemical, Shaoyuan Chemical Technology, Bailingwei Technology etc.

Unless otherwise specified in Examples, the reaction was carried out under N₂ atmosphere. Unless otherwise specified in the examples, the solution refers to an aqueous solution. Unless otherwise specified in Examples, the reaction temperature was room temperature. Unless otherwise specified in Examples, M refers to mole per liter.
DCM: dichloromethane; THF: tetrahydrofuran; DMF: N,N-dimethylformamide;
DIEA: N,N-diisopropylethylamine;
HATU: 2-(7-azobenzotriazole)-N,N,N′,N′-tetramethyluronium hexafluorophosphate;
TFA: trifluoroacetic acid; DIPEA: N, N-diisopropylethylamine.

### Intermediate Example 1: Intermediate IM-1 Synthesis

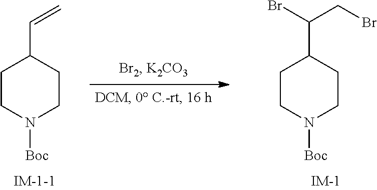

### Step 1, Compound IM-1 Synthesis

A single-necked flask was added with substrate IM-1-1 (1.0 g, 4.7 mmol). The mixture was dissolved with DCM (15 mL) under stirring. Then, the mixture was added at 0° C. with K₂CO₃ (1.4 g, 10.1 mmol) and Br₂ (1.5 g, 9.4 mmol), and reacted at room temperature for 16 hours, with TLC monitoring. After the completion of the reaction, the mixture was added with water and DCM to extract three times. The organic phase was combined, washed with saturated saline solution, and dried with anhydrous sodium sulfate, and then purified by medium pressure liquid chromatography, to give the product IM-1 (1.2 g, 3.2 mmol, 68.8% yield).

### Intermediate Example 2: Intermediate IM-2 Synthesis

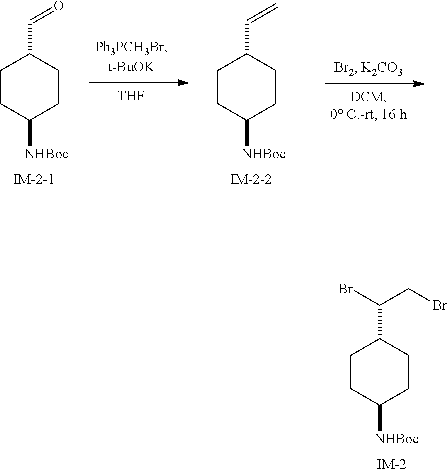

### Step 1, Compound IM-2-2 Synthesis

A single-necked flask was added with substrate Ph₃PCH₃Br (37.7 g, 105.6 mmol). The mixture was dissolved with ultra dry THE (150 mL) under stirring. Then, the mixture was added at 0° C. with t-BuOK (29.6 g, 264.3 mmol), stirred for 15 mins, and then added with IM-2-1 (20.0 g, 88.1 mmol), and reacted at room temperature for 1 hour, with TLC monitoring. After the completion of the reaction, the mixture was added with water to quench, and condensated under reduced pressure. The residue was dissolved with petroleum ether, and filtered. The filtrate was washed with saturated saline solution, and dried with anhydrous sodium sulfate and then condensated under reduced pressure, to give the crude product IM-2-2 (25.0 g, crude).

### Step 2, Compound IM-2 Synthesis

A single-necked flask was added with substrate IM-2-2 (25.0 g, crude). The mixture was dissolved with DCM (200 mL) under stirring. Then, the mixture was added at 0° C. with K₂CO₃ (25.5 g, 185.0 mmol) and Br₂ (28.2 g, 176.2 mmol), and reacted at room temperature for 16 hours, with TLC monitoring. After the completion of the reaction, the mixture was added with water and DCM to extract three times. The organic phase was combined, washed with saturated saline solution, and dried with anhydrous sodium sulfate, and then purified by medium pressure liquid chromatography, to give the product IM-2 (28.6 g, 74.3 mmol, 84.3% yield).

### Intermediate Example 3: Intermediate IM-3a and IM-3b Synthesis

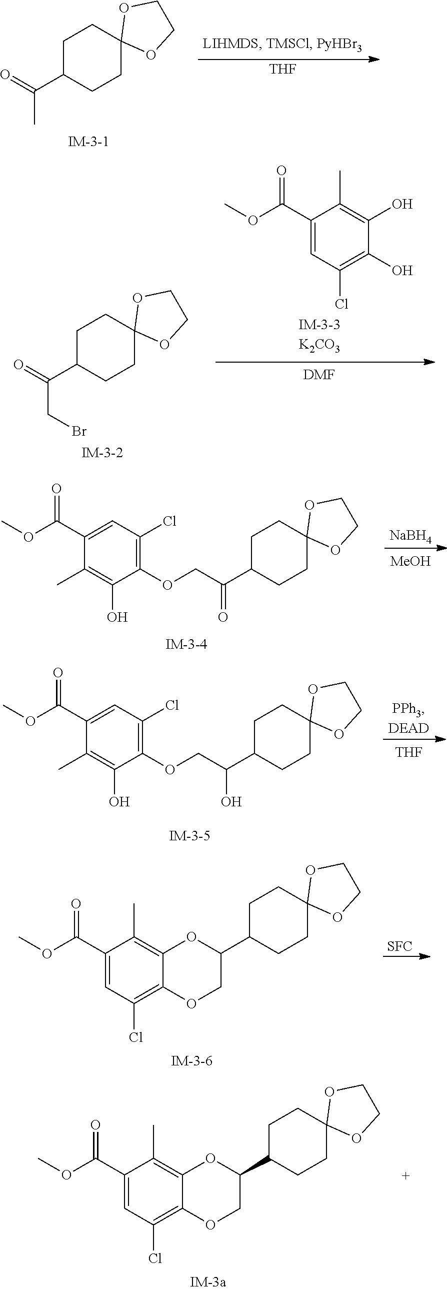

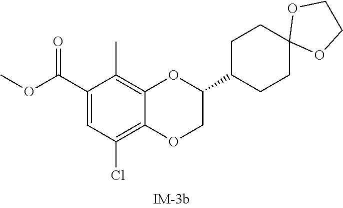 [SMILES: COC(=O)c1cc(Cl)c2c(c1C)O[C@H](C1CCC3(CC1)OCCO3)CO2]

### Step 1, Compound IM-3-2 Synthesis

Under nitrogen protection, a three-necked flask was added with substrate IM-3-1 (20.0 g, 108.7 mmol). The mixture was dissolved with THE (400 mL) under stirring, and then under nitrogen protection and at 0° C. added dropwise with LIHMDS (218.0 mmol, 167.7 mL), stirred at 0° C. for 30 mins, and then added with TMSCl (24.0 g, 221.0 mmol), further stirred at 0° C. for 1 hour, and then added with PyHBr₃ (53.0 g, 166.2 mmol), reacted at 0° C. for 1 hour, with TLC(PE:EA=10:1) monitoring. After the completion of the reaction, the reaction mixture was added to saturated sodium bicarbonate solution, and added with ethyl acetate to extract three times. The organic phase was combined, washed with saturated saline solution, and dried with anhydrous sodium sulfate, to give the crude product IM-3-2 (30.0 g, crude). LCMS (ESI+) m/z: 263.0, 265.0 [M+H]⁺

### Step 2, Compound IM-3-4 Synthesis

A single-necked flask was added with substrate IM-3-3 (19.0 g, 87.7 mmol). The mixture was dissolved with DMF (300 mL) under stirring, and then added with IM-3-2 (26.0 g, 98.8 mmol) and K₂CO₃ (40.0 g, 289.9 mmol), stirred at 50° C. to react for 1 hour, with LC-MS monitoring. After the completion of the reaction, the reaction mixture was adjusted with 1 N HCl solution to pH=6, added with ethyl acetate to extract three times. The organic phase was combined, washed with saturated saline solution, and dried with anhydrous sodium sulfate, to give the crude product IM-3-4 (39.6 g, crude). LCMS (ESI+) m/z: 397.0 [M−H]⁻

### Step 3, Compound IM-3-5 Synthesis

A single-necked flask was added with substrate IM-3-4 (39.0 g, 97.8 mmol). The mixture was dissolved with MeOH (300 mL) under stirring, and then at 0° C. added with NaBH₄ (4.0 g, 105.8 mmol), and stirred to react for 0.5 hours, with LC-MS monitoring. After the completion of the reaction, the reaction mixture was condensated under reduced pressure. Water was added to dissolve the crude product. The reaction mixture was adjusted with 1 N HCl solution to pH=6-7, added with ethyl acetate to extract three times. The organic phase was combined, washed with saturated saline solution, and dried with anhydrous sodium sulfate, and then purified by medium pressure liquid chromatography to give the product IM-3-5 (26.0 g, 64.9 mmol, 66.3% yield). LCMS (ESI⁺) m/z: 401.8 [M+H]⁺

### Step 4, Compound IM-3-6 Synthesis

A single-necked flask was added with substrate IM-3-5 (26.0 g, 64.9 mmol). The mixture was dissolved with THE (300 mL) under stirring, and then at 0° C. added with PPh₃ (21.0 g, 80.2 mmol) and DIAD (16.0 g, 79.1 mmol), stirred at 0° C. to react for 0.5 hours and then reacted at room temperature overnight, with LC-MS monitoring. After the completion of the reaction, the reaction mixture was condensated under reduced pressure, purified by medium pressure liquid chromatography to give the crude product IM-3-6 (26.0 g, crude). LCMS (ESI⁺) m/z: 383.8 [M+H]⁺

IM-3-6 can be resolved by SFC to give IM-3a and IM-3b, LCMS (ESI⁺) m/z: 383.1 [M+H]⁺.

IM-3a NMR: ¹H NMR (600 MHz, DMSO-d₆) δ 7.44 (s, 1H), 4.56 (dd, J=11.4, 2.2 Hz, 1H), 4.17-4.09 (m, 1H), 4.02-3.98 (m, 1H), 3.88-3.84 (m, 4H), 3.79 (s, 3H), 2.34 (s, 3H), 19.4-1.92 (m, 1H), 1.72-1.65 (m, 4H), 1.50-1.39 (m, 4H).

### Intermediate Example 4: Intermediate IM-4a, IM-4b, IM-4c and IM-4d Synthesis

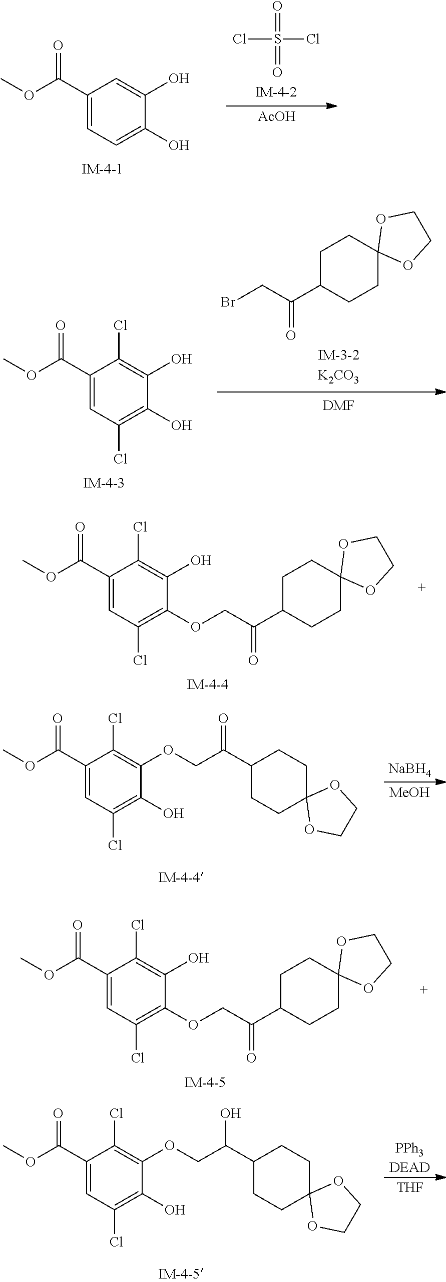

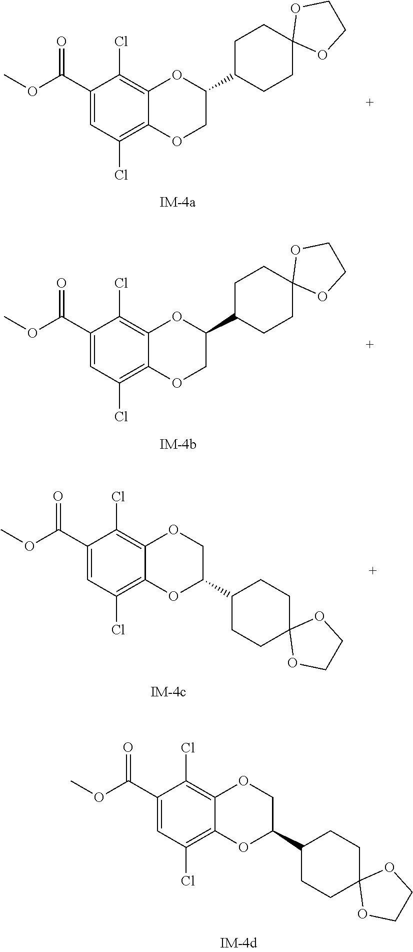 [SMILES: COC(=O)c1cc(Cl)c2c(c1Cl)O[C@H](C1CCC3(CC1)OCCO3)CO2 ; COC(=O)c1cc(Cl)c2c(c1Cl)O[C@@H](C1CCC3(CC1)OCCO3)CO2 ; COC(=O)c1cc(Cl)c2c(c1Cl)OC[C@H](C1CCC3(CC1)OCCO3)O2 ; COC(=O)c1cc(Cl)c2c(c1Cl)OC[C@@H](C1CCC3(CC1)OCCO3)O2]

A single-necked flask was added with substrate IM-4-1 (2.0 g, 11.9 mmol). The mixture was dissolved with AcOH (20 mL) under stirring, and then added dropwise with IM-4-2 (2.0 g, 14.8 mmol, 1.2 mL), stirred at 60° C. for 2 hours, with LC-MS monitoring. After the completion of the reaction, the reaction mixture was condensated under reduced pressure, added with water and ethyl acetate to extract three times. The organic phase was combined, washed with saturated saline solution, and dried with anhydrous sodium sulfate, to give the crude product IM-4-3 (2.1 g, crude). LCMS (ESI⁺) m/z: 237.0 [M+H]⁺

### Step 2, Compound IM-4-4 and IM-4-4′ Synthesis

Following the synthesis method of Example IM-3, Step 2, except replacing Step 2, IM-3-3 with IM-4-3 (2.1 g, 8.9 mmol), the same synthesis method was applied, to give the compound IM-4-4 and IM-4-4′ (1.8 g, 4.3 mmol, 48.3% yield), LCMS (ESI⁺) m/z: 419.3 [M+H]⁺.

### Step 3, Compound IM-4-5 and IM-4-5′ Synthesis

Following the synthesis method of Example IM-3, Step 3, except replacing Step 3, IM-3-4 with IM-4-4 and IM-4-4′ (1.8 g, 4.3 mmol), the same synthesis method was applied, to give the compound IM-4-5 and IM-4-5′(1.3 g, 3.1 mmol, 71.8 yield), LCMS (ESI⁺) m/z: 421.3 [M+H]⁺.

### Step 4, Compound IM-4a, IM-4b, IM-4c and IM-4d Synthesis

Following the synthesis method of Example IM-3, Step 4, except replacing Step 4, IM-3-5 with IM-4-5 and IM-4-5′ (1.3 g, 3.1 mmol), the same synthesis method was applied, and the obtained compound was resolved by SFC to give IM-4a (72.0 mg, 178.5 mol), IM-4b (170.0 mg, 421.5 mol), IM-4c (74.0 mg, 183.5 mol) and IM-4d (166.0 mg, 411.6 mol), LCMS (ESI⁺) m/z: 403.3 [M+H]⁺.

### Intermediate Example 5: Intermediate IM-5a, IM-5b, IM-5c and IM-5d

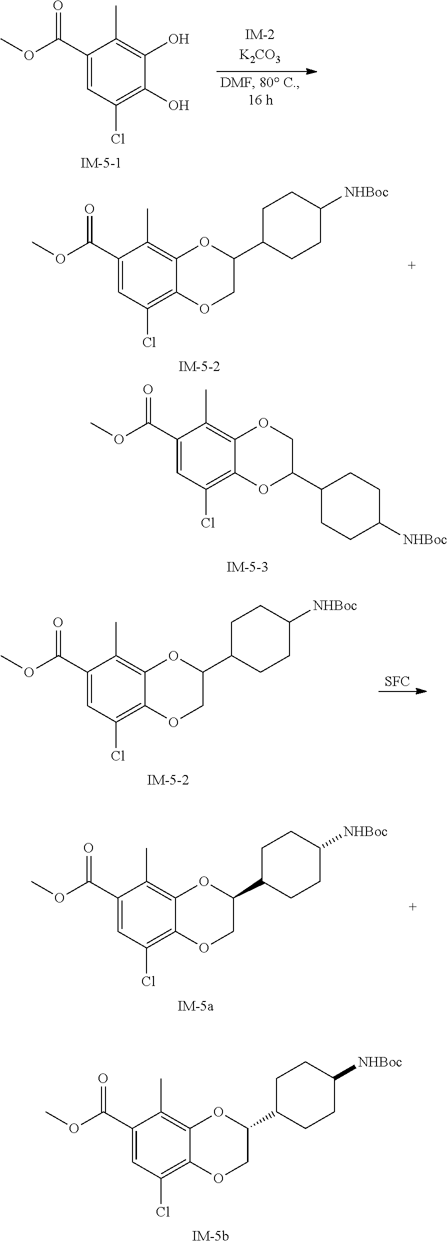

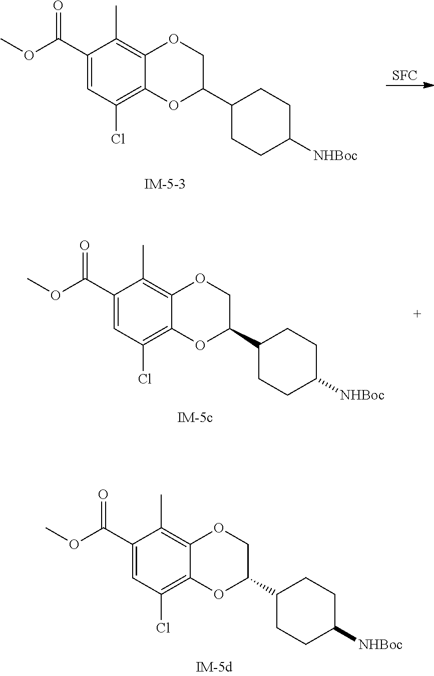

### Step 1, Compound IM-5-2 and IM-5-3 Synthesis

Following the synthesis method of Example IM-3, Step 2, except replacing Step 2, IM-3-3 with IM-5-1 (2.0 g, 9.2 mmol), the same synthesis method was applied, to give the compound IM-5-2 (1.0 g, 2.3 mmol, 24.7% yield), IM-5-3 (1.0 g, 2.3 mmol, 24.7% yield), LCMS (ESI⁺) m/z: 439.2 [M+H]⁺.

### Step 2, Compound IM-5a and IM-5b Synthesis

Compound IM-5a and IM-5b can be obtained by SFC resolution from Compound IM-5-2.

### Step 3, Compound IM-5c and IM-5d Synthesis

Compound IM-5c and IM-5d can be obtained by SFC resolution from Compound IM-5-3.

### Example 1, Compound 1 Synthesis

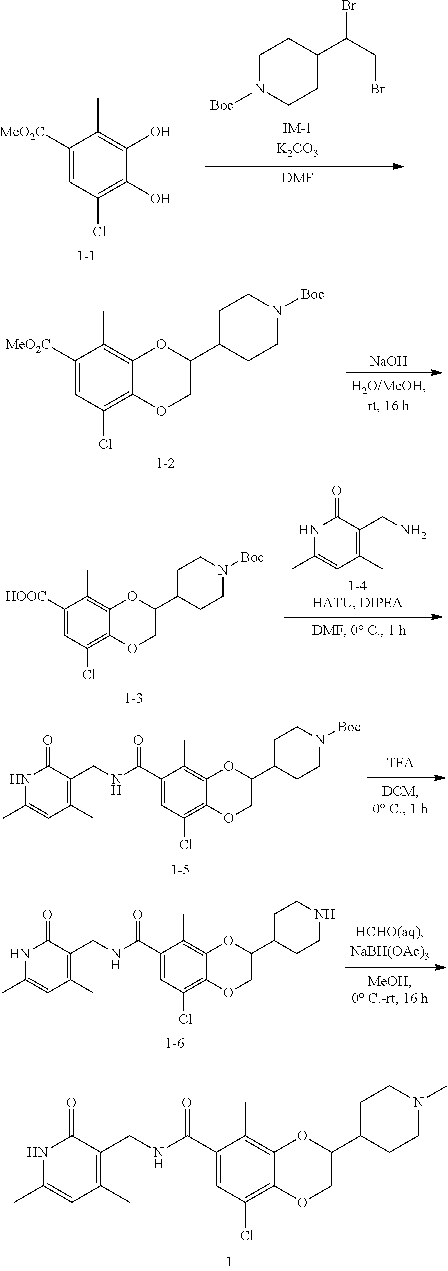

### Step 1, Compound 1-2 Synthesis

A single-necked flask was added with substrate 1-1 (200.0 mg, 0.9 mmol). The mixture was dissolved with DMF (5 mL) under stirring, and then added with IM-1 (411.2 mg, 1.1 mmol), heated to 80° C. to react for 16 hours, with TLC monitoring. After the completion of the reaction, the reaction mixture was cooled to room temperature, added with water and ethyl acetate to extract three times. The organic phase was combined, washed with saturated saline solution, and dried with anhydrous sodium sulfate, and then purified by medium pressure liquid chromatography, to give the product 1-2 (61.0 mg, 143.2 mol, 15.9% yield).

### Step 2, Compound 1-3 Synthesis

A single-necked flask was added with substrate 1-2 (61.0 mg, 143.2 mol), NaOH (11.5 mg, 287.5 mol). The mixture was dissolved with MeOH (2 mL) and water (2 mL) under stirring. Then, reacted at room temperature for 16 hours, with LC-MS monitoring. After the completion of the reaction, the reaction mixture was condensated under reduced pressure, added with water, and further extracted 6 times with dichloromethane/methanol (10/1 volume ratio) solution. The organic phase was combined, washed with saturated saline solution, and dried with anhydrous sodium sulfate, and then purified by medium pressure liquid chromatography, to give the product 1-3 (52.0 mg, 126.2 mol, 88.3% yield). LCMS (ESI⁺) m/z: 412.1 [M+H]⁺

### Step 3, Compound 1-5 Synthesis

A single-necked flask was added with substrate 1-3 (52.0 mg, 126.2 mol). The mixture was dissolved with DMF (2 mL) under stirring. Then, the mixture was added at 0° C. with DIPEA (49.0 mg, 0.4 mmol, 66.0 L), stirred for 5 mins, and then added with HATU (72.0 mg, 189.5 mol), further stirred for 5 mins, and then added with 1-4 (19.2 mg, 126.1 mol) to react for 1 hour, with LC-MS monitoring. After the completion of the reaction, the mixture was added with water to quench, the reaction mixture was condensated under reduced pressure, extracted with ethyl acetate three times. The organic phase was combined, washed with saturated saline solution, and dried with anhydrous sodium sulfate, and then purified by medium pressure liquid chromatography, to give the product 1-5 (67.8 mg, 124.2 mol). LCMS (ESI⁺) m/z: 546.2 [M+H]⁺

### Step 4, Compound 1-6 Synthesis

A single-necked flask was added with substrate 1-5 (67.8 mg, 124.2 mol). The mixture was dissolved with DCM (3 mL) under stirring. Then, the mixture was added at 0° C. with TFA (3 mL), under ice bath stirred for 1 hour, with LC-MS monitoring. After the completion of the reaction, the reaction mixture was condensated under reduced pressure, added with saturated sodium bicarbonate to quench, extracted with ethyl acetate three times. The organic phase was combined, washed with saturated saline solution, and dried with anhydrous sodium sulfate, and then purified by medium pressure liquid chromatography, to give the product 1-6 (20.0 mg, 44.9 mol, 36.2% yield). LCMS (ESI⁺) m/z: 446.2 [M+H]⁺

### Step 5, Compound 1 Synthesis

A single-necked flask was added with substrate 1-6 (20.0 mg, 44.9 mol). The mixture was dissolved with MeOH (2 mL) under stirring. Then, the mixture was added at 0° C. with NaBH(OAc)₃ (19.1 mg, 0.09 mmol), formaldehyde (45.0 mg, 37% wt, 1.5 mmol), stirred at room temperature for 16 hours, with LC-MS monitoring. After the completion of the reaction, the mixture was added with water to quench, the reaction mixture was condensated under reduced pressure, extracted with ethyl acetate three times. The organic phase was combined, washed with saturated saline solution, and dried with anhydrous sodium sulfate, and then purified by medium pressure liquid chromatography, to give the product 1 (3.0 mg, 6.5 mol, 14.5% yield). LCMS (ESI⁺) m/z: 460.2 [M+H]⁺

¹H NMR (600 MHz, Methanol-d₄) δ 6.93 (m, 1H), 6.10 (s, 1H), 4.58-4.56 (m, 1H), 4.43-4.41 (m, 3H), 4.10-4.05 (m, 1H), 3.98-3.92 (m, 1H), 3.02-2.96 (m, 2H), 2.35 (s, 3H), 2.33-2.31 (m, 3H), 2.23 (s, 3H), 2.17 (s, 3H), 2.02-1.95 (m, 1H), 1.80-1.75 (m, 1H), 1.72-1.68 (m, 1H), 1.62-1.56 (m, 2H).

### Example 2, Compound 2 Synthesis

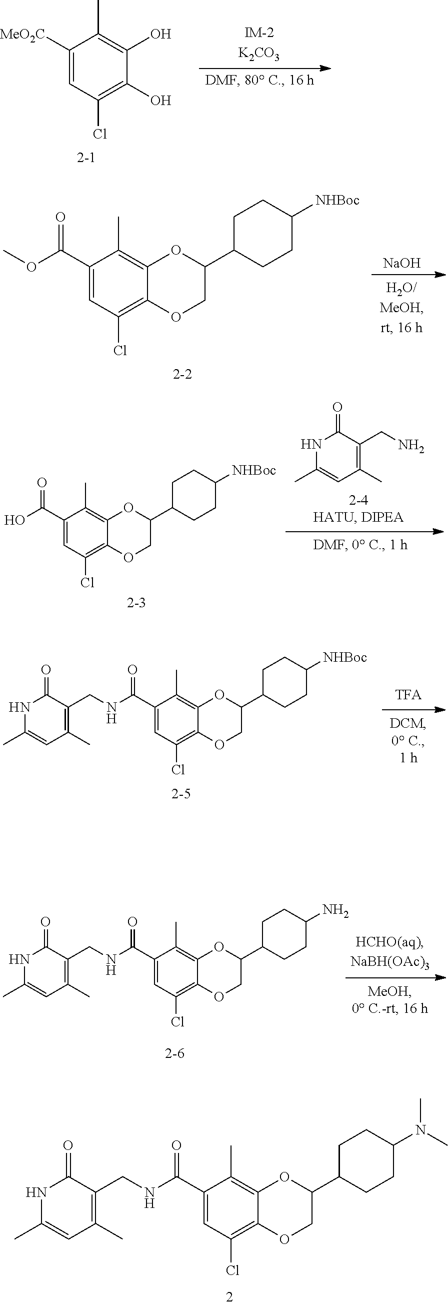

### Step 1, Compound 2-2 Synthesis

A single-necked flask was added with substrate 2-1 (1.2 g, 5.5 mmol). The mixture was dissolved with DMF (20 mL) under stirring, and then added with IM-2 (2.6 g, 6.8 mmol), heated to 80° C. to react for 16 hours, with TLC monitoring. After the completion of the reaction, the reaction mixture was returned to room temperature, added with water and ethyl acetate to extract three times. The organic phase was combined, washed with saturated saline solution, and dried with anhydrous sodium sulfate, and then purified by medium pressure liquid chromatography, to give the product 2-2 (220.0 mg, 0.5 mmol, 9.2% yield).

### Step 2, Compound 2-3 Synthesis

A single-necked flask was added with substrate 2-2 (220.0 mg, 0.5 mmol), NaOH (40.0 mg, 1.0 mmol). The mixture was dissolved with MeOH (4 mL) and water (4 mL) under stirring. Then, reacted at room temperature for 16 hours, with LC-MS monitoring. After the completion of the reaction, the reaction mixture was condensated under reduced pressure, added with water, and further extracted 6 times with dichloromethane/methanol (10/1 volume ratio) solution. The organic phase was combined, washed with saturated saline solution, and dried with anhydrous sodium sulfate, and then purified by medium pressure liquid chromatography, to give the product 2-3 (212.0 mg, 0.5 mmol, 99.5% yield). LCMS (ESI⁺) m/z: 426.2 [M+H]⁺

### Step 3, Compound 2-5 Synthesis

A single-necked flask was added with substrate 2-3 (212.0 mg, 0.5 mmol). The mixture was dissolved with DMF (5 mL) under stirring. Then, the mixture was added at 0° C. with DIPEA (66.8 mg, 0.5 mmol, 90.0 L), stirred for 5 mins, and then added with HATU (196.3 mg, 0.5 mmol), further stirred for 5 mins, and then added with 2-4 (94.3 mg, 0.6 mmol) to react for 1 hour, with LC-MS monitoring. After the completion of the reaction, the mixture was added with water to quench, and the reaction mixture was condensated under reduced pressure, extracted with ethyl acetate three times. The organic phase was combined, washed with saturated saline solution, and dried with anhydrous sodium sulfate, and then purified by medium pressure liquid chromatography, to give the product 2-5 (110.0 mg, 0.2 mmol, 39.3% yield). LCMS (ESI⁺) m/z: 560.2 [M+H]⁺

### Step 4, Compound 2-6 Synthesis

A single-necked flask was added with substrate 2-5 (110.0 mg, 0.2 mmol). The mixture was dissolved with DCM (5 mL) under stirring. Then, the mixture was added at 0° C. with TFA (5 mL), stirred at room temperature for 1 hour, with LC-MS monitoring. After the completion of the reaction, the reaction mixture was condensated under reduced pressure, added with saturated sodium bicarbonate to quench, extracted with ethyl acetate three times. The organic phase was combined, washed with saturated saline solution, and dried with anhydrous sodium sulfate, and then purified by medium pressure liquid chromatography, to give the product 2-6 (40.0 mg, 0.09 mmol, 43.5% yield). LCMS (ESI⁺) m/z: 460.2 [M+H]⁺

### Step 5, Compound 2 Synthesis

A single-necked flask was added with substrate 2-6 (40.0 mg, 0.09 mmol). The mixture was dissolved with MeOH (3 mL) under stirring. Then, the mixture was added at 0° C. with NaBH(OAc)₃ (36.9 mg, 0.2 mmol), formaldehyde (87.0 mg, 37% wt, 0.9 mmol), stirred at room temperature for 16 hours, with LC-MS monitoring. After the completion of the reaction, the mixture was added with water to quench, the reaction mixture was condensated under reduced pressure, extracted with ethyl acetate three times. The organic phase was combined, washed with saturated saline solution, and dried with anhydrous sodium sulfate, and then purified by medium pressure liquid chromatography, to give the product 2 (2.0 mg, 0.04 mmol, 4.6% yield). LCMS (ESI⁺) m/z: 488.3 [M+H]⁺

¹H NMR (600 MHz, CDCl₃) δ 9.51 (s, 1H), 7.08-7.03 (m, 1H), 6.97 (s, 1H), 5.92 (s, 1H), 4.50-4.46 (m, 2H), 4.36-4.32 (m, 1H), 4.08-4.01 (m, 1H), 3.91-3.85 (m, 1H), 2.91-2.89 (m, 1H), 2.68-2.66 (m, 6H), 2.38 (s, 3H), 2.24 (s, 3H), 2.20 (s, 3H), 2.12-2.08 (m, 1H), 1.97-1.93 (m, 1H), 1.80-1.60 (m, 7H).

### Example 3, Compound 3 Synthesis

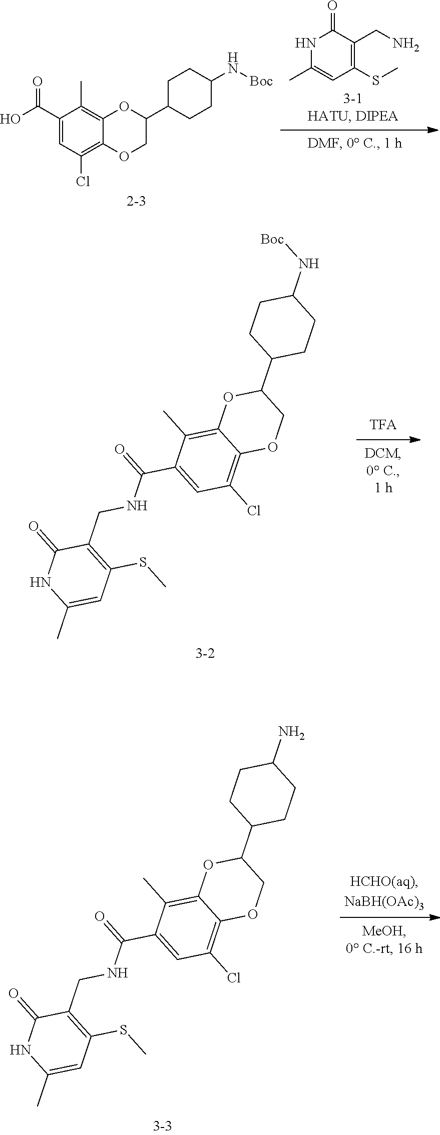

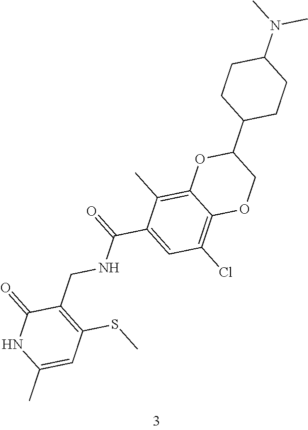 [SMILES: CSc1cc(C)[nH]c(=O)c1CNC(=O)c1cc(Cl)c2c(c1C)OC(C1CCC(N(C)C)CC1)CO2]

### Step 1, Compound 3-2 Synthesis

A single-necked flask was added with substrate 2-3 (125.0 mg, 0.3 mmol). The mixture was dissolved with DMF (5 mL) under stirring. Then, the mixture was added at 0° C. with DIPEA (113.8 mg, 0.9 mmol, 153.4 L), stirred for 5 mins, and then added with HATU (111.5 mg, 0.3 mmol), further stirred for 5 mins, and then added with 3-1 (64.9 mg, 0.4 mmol) to react for 1 hour, with LC-MS monitoring. After the completion of the reaction, the mixture was added with water to quench, the reaction mixture was condensated under reduced pressure, extracted with ethyl acetate three times. The organic phase was combined, washed with saturated saline solution, and dried with anhydrous sodium sulfate, and then purified by medium pressure liquid chromatography, to give the product 3-2 (50.0 mg, 84.4 mol, 28.8% yield). LCMS (ESI⁺) m/z: 592.2 [M+H]⁺

### Step 2, Compound 3-3 Synthesis

A single-necked flask was added with substrate 3-2 (50.0 mg, 0.08 mmol). The mixture was dissolved with DCM (2 mL) under stirring. Then, the mixture was added at 0° C. with TFA (2 mL), under ice bath stirred for 1 hour, with LC-MS monitoring. After the completion of the reaction, the reaction mixture was condensated under reduced pressure, added with saturated sodium bicarbonate to quench, extracted with ethyl acetate three times. The organic phase was combined, washed with saturated saline solution, and dried with anhydrous sodium sulfate, and then purified by medium pressure liquid chromatography, to give the product 3-3 (31.0 mg, 63.0 mol, 74.7% yield). LCMS (ESI⁺) m/z: 492.2 [M+H]⁺

### Step 3, Compound 3 Synthesis

A single-necked flask was added with substrate 3-3 (31.0 mg, 63.0 mol). The mixture was dissolved with MeOH (2 mL) under stirring. Then, the mixture was added at 0° C. with NaBH(OAc)₃ (35.7 mg, 168.5 mol), formaldehyde (84.4 mg, 37% wt, 844 mol) stirred at room temperature for 1 hour, with LC-MS monitoring. After the completion of the reaction, the mixture was added with water to quench, the reaction mixture was condensated under reduced pressure, extracted with ethyl acetate three times. The organic phase was combined, washed with saturated saline solution, and dried with anhydrous sodium sulfate, and then purified by medium pressure liquid chromatography, to give the product 3 (20.0 mg, 38.5 mol, 61.0% yield). LCMS (ESI⁺) m/z: 520.3 [M+H]⁺

¹H NMR (600 MHz, Methanol-d₄) δ 6.96 (s, 1H), 6.28 (s, 1H), 4.51-4.49 (m, 2H), 4.43-4.40 (m, 1H), 4.10-4.05 (m, 1H), 3.98-3.93 (m, 1H), 3.26-3.20 (m, 1H), 2.85 (s, 6H), 2.53 (s, 3H), 2.28 (s, 3H), 2.28-2.22 (m, 1H), 2.21 (s, 3H), 2.18-2.14 (m, 2H), 2.04-1.98 (m, 1H), 1.76-1.71 (m, 1H), 1.61-1.49 (m, 2H), 1.48-1.35 (m, 2H).

### Example 4, Compound 4 Synthesis

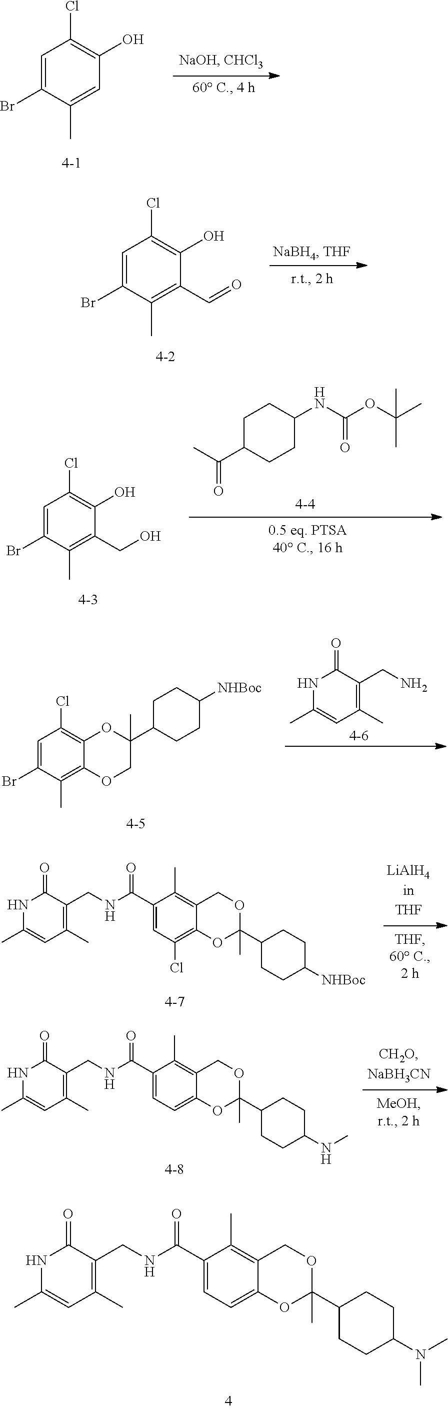

### Step 1, Compound 4-2 Synthesis

A single-necked flask was added with substrate 4-1 (2.2 g, 10.0 mmol), dissolved with chloroform (20 mL), added with ethanol (20 mL) and 4 M NaOH solution (20 mL), heated to 60° C. and stirred to react for 4 hours. After cooling, the reaction mixture was adjusted by diluted hydrochloric acid to pH=6, added with water and DCM to extract three times. The organic phase was combined, washed with saturated saline solution, and dried with anhydrous sodium sulfate, and then purified by column chromatography (PE:EA=1:1), to give the product 4-2 (1.2 g, 51.0% yield) as yellow solid.

### Step 2, Compound 4-3 Synthesis

A single-necked flask was added with substrate 4-2 (1.2 g, 5.0 mmol), dissolved with THF (10 mL), slowly added with NaBH₄, stirred at room temperature for 2 hours. After the completion of the reaction, the reaction mixture was added with saturated ammonium chloride solution to quench the reaction, and extracted with DCM two times. The organic phase was combined and dried with anhydrous sodium sulfate, and then filtered. The filtrate was concentrated. The residue was purified by column chromatography (DCM:MeOH=10:1) to give 4-3 (1.1 g, 91.0% yield) as yellow solid.

### Step 3, Compound 4-5 Synthesis

A single-necked flask was added with substrate 4-3 (500.0 mg, 2.0 mmol), 4-4 (580.0 mg, 2.4 mmol), PTSA (172.0 mg, 1.0 mmol) to dissolve in toluene (5 mL). The reaction mixture was heated to 40° C. and reacted overnight. After the completion of the reaction, the reaction mixture was filtered. The filtrate was concentrated. The residue was purified by column chromatography (DCM:MeOH=20:1) to give 4-5 (120.0 mg, 13.0% yield) as yellow solid.

### Step 4, Compound 4-7 Synthesis

A single-necked flask was added with substrate 4-5 (100.0 mg, 0.2 mmol), 4-6 (94.0 mg, 0.6 mmol), Molybdenum hexacarbonyl (55.0 mg, 0.2 mmol), Pd(dppf)Cl₂.DCM (20.0 mg, 0.02 mmol), K₂CO₃ (50.0 mg, 0.4 mmol), to dissolve in 1,4-dioxane (20 mL). Under nitrogen protection, the reaction mixture was heated to 110° C. and reacted overnight. After the cooling of reaction mixture, the reaction mixture was filtered. The filtrate was concentrated. The residue was purified by column chromatography to give 4-7 (65.0 mg, 53.0% yield) as brown solid.

### Step 5, Compound 4-8 Synthesis

A single-necked flask was added with substrate 4-7 (50.0 mg, 0.09 mmol) to dissolve in THE (10 mL), and added with 1 N LiAlH₄ solution (10 mL), heated to 60° C. to react for 2 hours. After cooling of the reaction mixture under ice bath condition, the reaction mixture was added with water (0.5 mL), 15% NaOH solution (0.5 mL), water (1.5 mL). The reaction mixture was sufficiently stirred, and then filtered. The filtrate was concentrated. The residue was purified by column chromatography (DCM:MeOH=10:1) to give 4-8 (15.0 mg, 35.0% yield) as off-white solid.

### Step 6, Compound 4 Synthesis

A single-necked flask was added with substrate 4-8 (10.0 mg, 0.02 mmol) to dissolve in methanol (5 mL), added with formaldehyde solution (20 mg). The reaction mixture was sufficiently stirred, and then added with NaBH₃CN (6.0 mg, 0.1 mmol), stirred at room temperature for 2 [symbol] . After the completion of the reaction, the reaction was quenched with water, added with DCM to extract two times. The organic phase was combined, and dried with anhydrous sodium sulfate, and then filtered. The filtrate was concentrated. The residue was purified by Prep-MPLC to give 4 (3.0 mg, 27.0% yield) as white solid product. LCMS (ESI⁺) m/z: 502.2 [M+H]⁺

¹H NMR (600 MHz, Methanol-d₄) δ¹H NMR (600 MHz, Methanol-d₄) δ 7.15 (d, J=8.4 Hz, 1H), 6.63 (d, J=8.4 Hz, 1H), 6.10 (s, 1H), 4.81-4.73 (m, 2H), 4.44 (s, 2H), 2.36 (s, 3H), 2.35-2.28 (m, 7H), 2.23 (s, 3H), 2.15 (s, 3H), 2.04-1.97 (m, 4H), 1.73-1.70 (m, 1H), 1.36 (s, 3H), 1.33-1.18 (m, 4H).

### Example 5, Compound 5 Synthesis

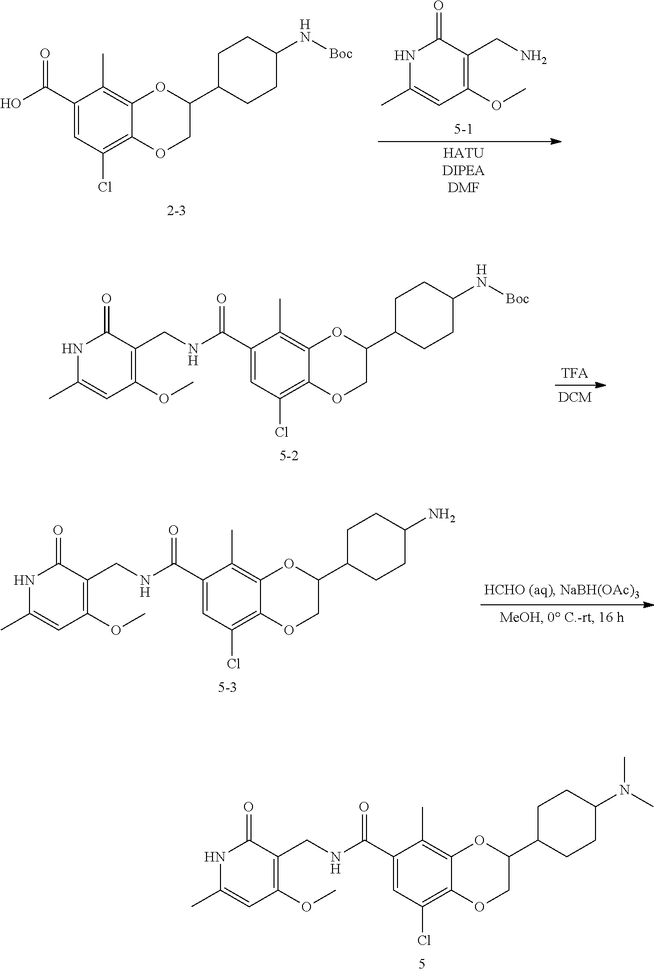

### Step 1, Compound 5-2 Synthesis

Following the synthesis method of Example 3, Step 1, except replacing Step 1, 3-1 with 5-1 (26.0 mg, 154.6 mol), the same synthesis method was applied, to give Compound 5-2 (51.0 mg, 88.5 mol, 86.0% yield), LCMS (ESI⁺) m/z: 576.1 [M+H]⁺

### Step 2, Compound 5-3 Synthesis

Following the synthesis method of Example 3, Step 2, except replacing Step 2, 3-2 with 5-2 (41.0 mg, 71.2 mol), the same synthesis method was applied, to give Compound 5-3 (33.0 mg, 69.3 mol, 97.3% yield), LCMS (ESI⁺) m/z: 476.0 [M+H]⁺

### Step 3, Compound 5 Synthesis

Following the synthesis method of Example 3, Step 3, except replacing Step 3, 3-3 with 5-3 (33.0 mg, 69.3 mol), the same synthesis method was applied, to give Compound 5 (15.0 mg, 29.8 mol, 43.0% yield), LCMS (ESI⁺) m/z: 468.2 [M+H]⁺.

¹H NMR (600 MHz, DMSO-d₆) δ 11.42 (s, 1H), 7.92 (t, J=4.8 Hz, 1H), 6.87 (s, 1H), 6.87 (s, 1H), 6.08 (s, 1H), 4.46-4.43 (m, 1H), 4.17 (s, J=4.8 Hz, 2H), 4.08-4.04 (m, 1H), 3.94-3.89 (m, 1H), 3.80 (s, 3H), 2.23-2.21 (m, 6H), 2.17 (s, 3H), 2.12 (s, 3H), 2.08-1.95 (m, 1H), 1.80-1.73 (m, 2H), 1.58-1.49 (m, 1H), 1.48-1.46 (m, 1H), 1.25-1.12 (m, 4H).

### Example 6, Compound 6 and 6′ Synthesis

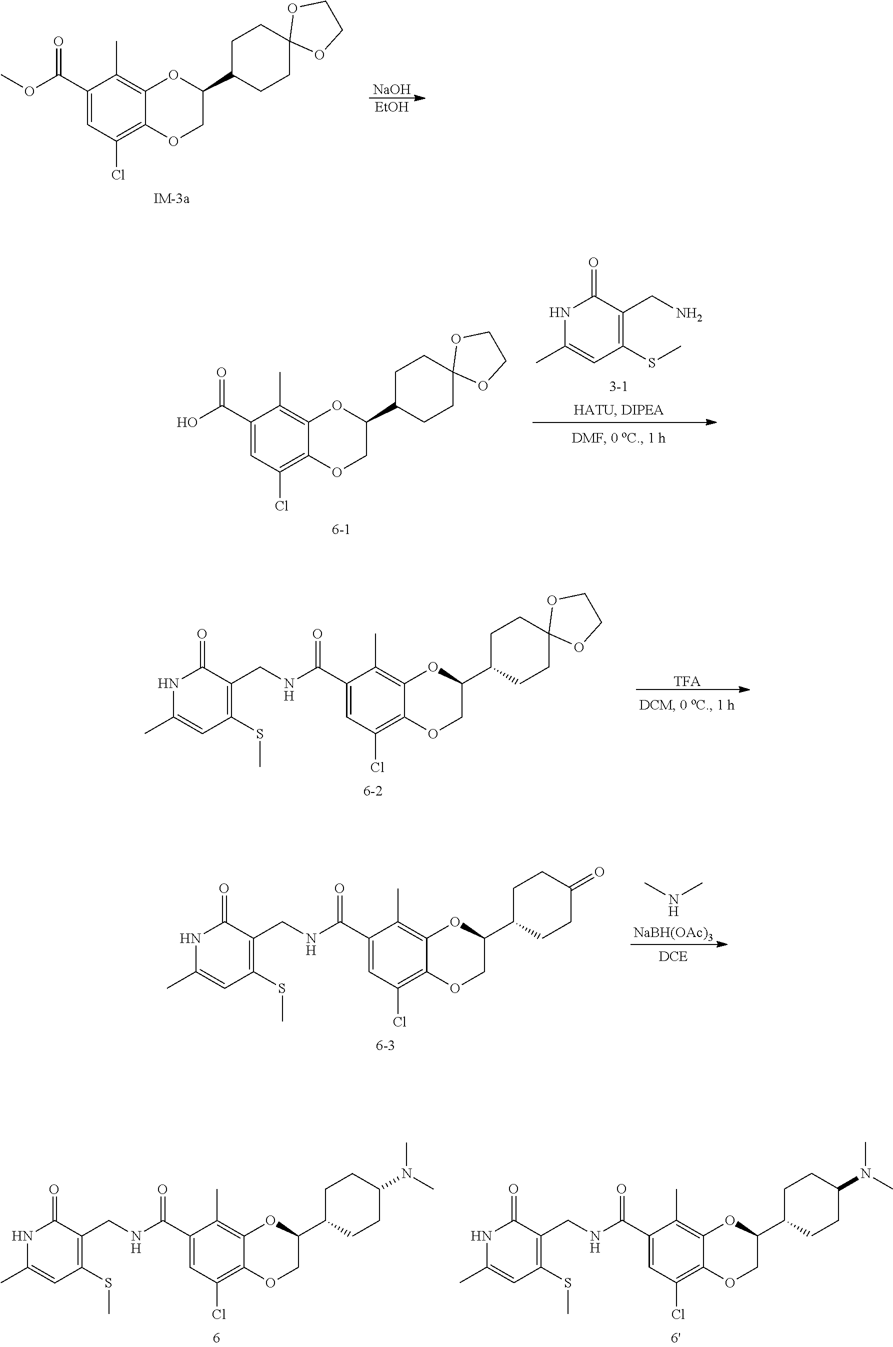

### Step 1, Compound 6-1 Synthesis

A single-necked flask was added with substrate IM-3a (1.5 g, 3.9 mmol). The mixture was dissolved with EtOH (10 mL) to dissolve, and then added with 2 N NaOH (10 mL), 80° C. to react for 1 hour, with LC-MS monitoring. After the completion of the reaction, the mixture was added with 1 N HCl to adjust to pH=6-7, and then added with EA solution to extract three times. The organic phase was combined, washed with saturated saline solution, and dried with anhydrous sodium sulfate and then condensated under reduced pressure, to give the crude product 6-1 (1.5 g, crude). LCMS (ESI⁺) m/z: 369.1 [M+H]⁺

### Step 2, Compound 6-2 Synthesis

Following the synthesis method of Example 3, Step 1, except replacing Step 1, 2-3 with 6-1 (250.0 mg, 1.4 mmol), 2-4 with 3-1, the same synthesis method was applied, to give Compound 6-2 (292.0 mg, 545.7 mol), LCMS (ESI⁺) m/z: 535.1 [M+H]⁺.

### Step 3, Compound 6-3 Synthesis

Following the synthesis method of Example 3, Step 2, except replacing Step 2, 3-2 with 6-2 (292.0 mg, 545.7 mol), the same synthesis method was applied, to give Compound 6-3 (250.0 mg, crude), LCMS (ESI⁺) m/z: 491.0 [M+H]⁺.

### Step 4, Compound 6 and 6′ Synthesis

A single-necked flask was added with substrate 6-3 (48.0 mg, 97.8 mol). The mixture was dissolved with DCE (2 mL) to dissolve, and then at 0° C. added with NaBH(OAc)₃ (103.0 mg, 0.5 mmol) and dimethylamine (22.0 mg, 0.5 mmol), and reacted at room temperature for 2 hours, with LC-MS monitoring. After the completion of the reaction, the mixture was added with water to quench, and then added with EA solution to extract three times. The organic phase was combined, washed with saturated saline solution, and dried with anhydrous sodium sulfate and then condensated under reduced pressure. The residue was purified by Prep-MPLC, to give the product 6 (6.3 mg, 0.01 mmol, 12.4% yield) and 6′(6.3 mg, 0.01 mmol, 12.4% yield). LCMS (ESI⁺) m/z: 520.5 [M+H]⁺

Compound 6 NMR: ¹H NMR (600 MHz, DMSO-d₆) δ 11.52 (s, 1H), 8.01 (t, J=4.8 Hz, 1H), 6.90 (s, 1H), 6.08 (s, 1H), 4.48 (dd, J=11.4, 1.8 Hz, 1H), 4.28 (d, J=11.4, 4.2 Hz, 2H), 4.09-4.05 (m, 1H), 3.94-3.91 (m, 1H), 2.45 (s, 3H), 2.42-2.35 (m, 1H), 2.32-2.30 (m, 6H), 2.16 (s, 3H), 2.14 (s, 3H), 2.05-2.01 (m, 1H), 1.92-1.85 (m, 2H), 1.82-1.88 (m, 1H), 1.59-1.52 (m, 1H), 1.26-1.18 (m, 4H).

### Example 7, Compound 7 Synthesis

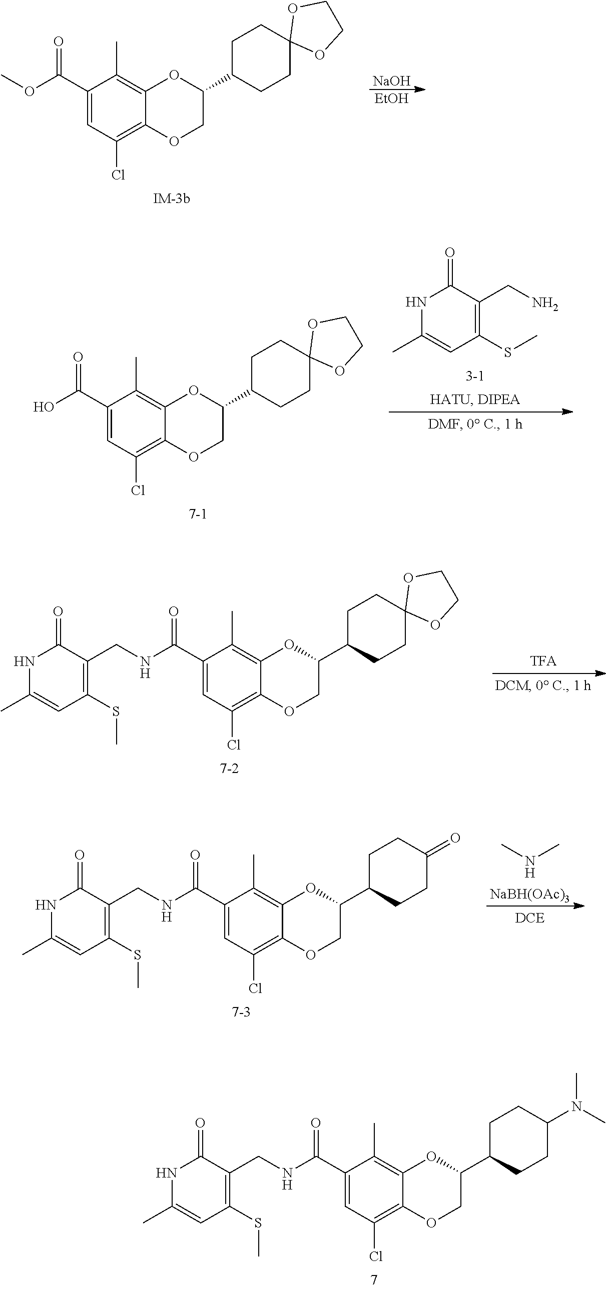

### Step 1, Compound 7-1 Synthesis

A single-necked flask was added with substrate IM-3b (1.5 g, 3.9 mmol). The mixture was dissolved with EtOH (10 mL) to dissolve, and then added with 2 N NaOH (10 mL), 80° C. to react for 1 hour, with LC-MS monitoring. After the completion of the reaction, the mixture was added with 1 N HCl to adjust to pH=6-7, and then added with EA solution to extract three times. The organic phase was combined, washed with saturated saline solution, and dried with anhydrous sodium sulfate and then condensated under reduced pressure, to give the crude product 7-1 (1.5 g, crude). LCMS (ESI⁺) m/z: 369.1 [M+H]⁺

### Step 2, Compound 7-2 Synthesis

Following the synthesis method of Example 3, Step 1, except replacing Step 1, 2-3 with 7-1 (250.0 mg, 677.9 mol), the same synthesis method was applied, to give Compound 7-2 (292.0 mg, 546.0 mol), LCMS (ESI⁺) m/z: 535.1 [M+H]⁺.

### Step 3, Compound 7-3 Synthesis

Following the synthesis method of Example 3, Step 2, except replacing Step 2, 3-2 with 7-2 (292.0 mg, 546.0 mol), the same synthesis method was applied, to give Compound 7-3 (250.0 mg, crude), LCMS (ESI⁺) m/z: 491.0 [M+H]⁺.

### Step 4, Compound 7 Synthesis

A single-necked flask was added with substrate 7-3 (48.0 mg, 97.8 mol). The mixture was dissolved with DCE (2 mL) to dissolve, and then at 0° C. added with NaBH(OAc)₃ (103.0 mg, 0.5 mmol) and dimethylamine (22.0 mg, 0.5 mmol), and reacted at room temperature for 2 hours, with LC-MS monitoring. After the completion of the reaction, the mixture was added with water to quench, and then added with EA solution to extract three times. The organic phase was combined, washed with saturated saline solution, and dried with anhydrous sodium sulfate and then condensated under reduced pressure. The residue was purified by medium pressure liquid chromatography, to give the product 7 (12.6 mg, 24.2 mol, 24.8% yield). LCMS (ESI⁺) m/z: 520.3 [M+H]⁺

Compound 7′H NMR (600 MHz, DMSO-d₆) δ 11.53 (s, 1H), 8.03-8.04 (m, 1H), 6.91 (s, 1H), 6.08 (s, 1H), 4.45-4.42 (dd, J=9.6, 1.8 Hz, 1H), 4.27 (d, J=4.8 Hz, 2H), 4.12-4.08 (m, 1H), 3.96-3.92 (m, 1H), 2.88-2.71 (m, 1H), 2.58-2.51 (m, 6H), 2.45 (s, 3H), 2.18-2.14 (m, 6H), 2.08-2.02 (m, 1H), 1.98-1.96 (m, 2H), 1.88-1.84 (m, 1H), 1.64-1.66 (m, 1H), 1.38-1.30 (m, 2H), 1.28-1.21 (m, 2H).

### Example 8, Compound 8 and 9 Synthesis

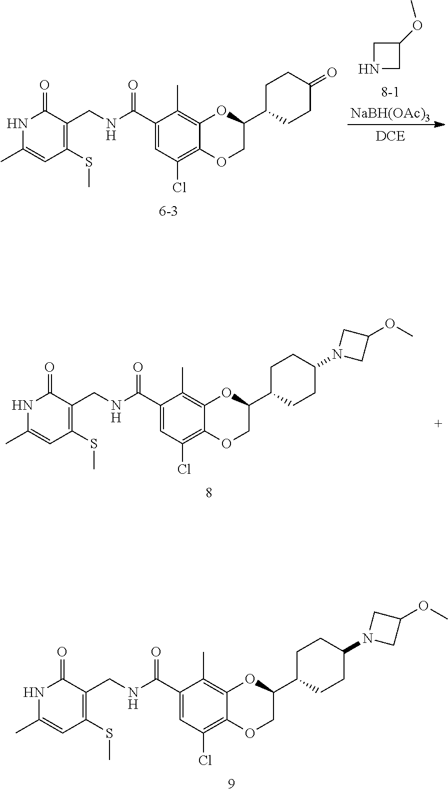

### Step 1, Compound 8 and 9 Synthesis

Following the synthesis method of Example 6, Step 4, except replacing Step 4, dimethylamine with 8-1 (35.5 mg, 0.4 mmol), the same synthesis method was applied, and further resolved by Prep-MPLC to give Compound 8 (21.0 mg, 37.4 mol, 18.0% yield) and 9 (20.0 mg, 35.6 mol, 17.0% yield), LCMS (ESI⁺) m/z: 562.2 [M+H]⁺.

Compound 8 NMR: ¹H NMR (600 MHz, DMSO-d₆) δ 11.50 (s, 1H), 8.03-8.01 (m, 1H), 6.89 (s, 1H), 6.08 (s, 1H), 4.47-4.42 (m, 1H), 4.27 (d, J=4.2 Hz, 2H), 4.16-4.12 (m, 1H), 3.91-3.86 (m, 2H), 3.46-3.42 (m, 2H), 3.13 (s, 3H), 2.72-2.70 (m, 2H), 2.45 (s, 3H), 2.16 (s, 3H), 2.13 (s, 3H), 1.97-1.93 (m, 1H), 1.90-1.84 (m, 1H), 1.79-1.69 (m, 2H), 1.59-1.50 (m, 2H), 1.20-1.11 (m, 2H), 0.89-0.79 (m, 2H).

Compound 9 NMR: ¹H NMR (600 MHz, DMSO-d₆) δ 11.52 (s, 1H), 8.03-8.01 (m, 1H), 6.89 (s, 1H), 6.08 (s, 1H), 4.44-4.40 (m, 1H), 4.27 (d, J=4.6 Hz, 2H), 4.09-4.05 (m, 1H), 3.95-3.90 (m, 2H), 3.46-3.43 (m, 2H), 3.15 (s, 3H), 2.68-2.62 (m, 2H), 2.45 (s, 3H), 2.38-2.16 (s, 3H), 2.13 (s, 3H), 1.65-1.55 (m, 5H), 1.51-1.38 (m, 3H), 1.32-1.29 (m, 2H).

### Example 9, Compound 10 and 11 Synthesis

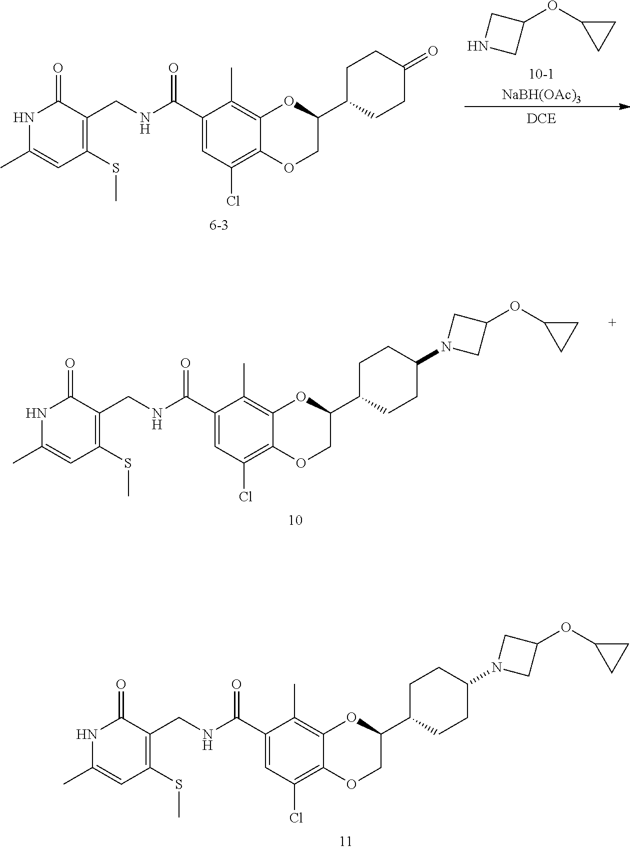

### Step 1, Compound 10 and 11 Synthesis

Following the synthesis method of Example 6, Step 4, except replacing Step 4, dimethylamine with 10-1 (14.0 mg, 123.7 mol), the same synthesis method was applied, and further resolved by Prep-MPLC to give Compound 10 (2.0 mg, 0.003 mmol, 20.0% yield) and 11 (2.0 mg, 0.003 mmol, 20.0% yield), LCMS (ESI⁺) m/z: 588.2 [M+H]⁺.

Compound 10 NMR: ¹H NMR (600 MHz, DMSO-d₆) δ 11.52 (s, 1H), 8.01 (t, J=4.2 Hz, 1H), 6.89 (s, 1H), 6.08 (s, 1H), 4.40-4.42 (m, 1H), 4.27 (d, J=4.8 Hz, 2H), 4.09-4.06 (m, 1H), 3.99-3.97 (m, 1H), 3.87-3.84 (m, 2H), 3.31-3.30 (m, 2H), 2.66 (s, 2H), 2.48-2.46 (m, 3H), 2.44 (s, 3H), 2.40-2.29 (m, 2H), 2.16 (s, 3H), 2.13 (s, 3H), 1.80-1.78 (m, 2H), 1.67-1.58 (m, 4H), 1.43-1.40 (m, 3H).

Compound 11 NMR: ¹H NMR (600 MHz, DMSO-d₆) δ 11.52 (s, 1H), 8.01 (t, J=4.6 Hz, 1H), 6.89 (s, 1H), 6.08 (s, 1H), 4.40-4.42 (m, 1H), 4.27 (d, J=4.8 Hz, 2H), 4.09-4.06 (m, 1H), 3.92-3.89 (m, 1H), 3.72-3.68 (m, 2H), 3.19-3.30 (m, 3H), 2.70-2.60 (m, 4H), 2.50 (s, 3H), 2.40-2.29 (m, 2H), 2.16 (s, 3H), 2.13 (s, 3H), 2.01-1.90 (m, 3H), 1.80-1.75 (m, 1H), 1.60-1.50 (m, 1H), 1.01-1.25 (m, 4H).

### Example 10, Compound 12 and 13 Synthesis

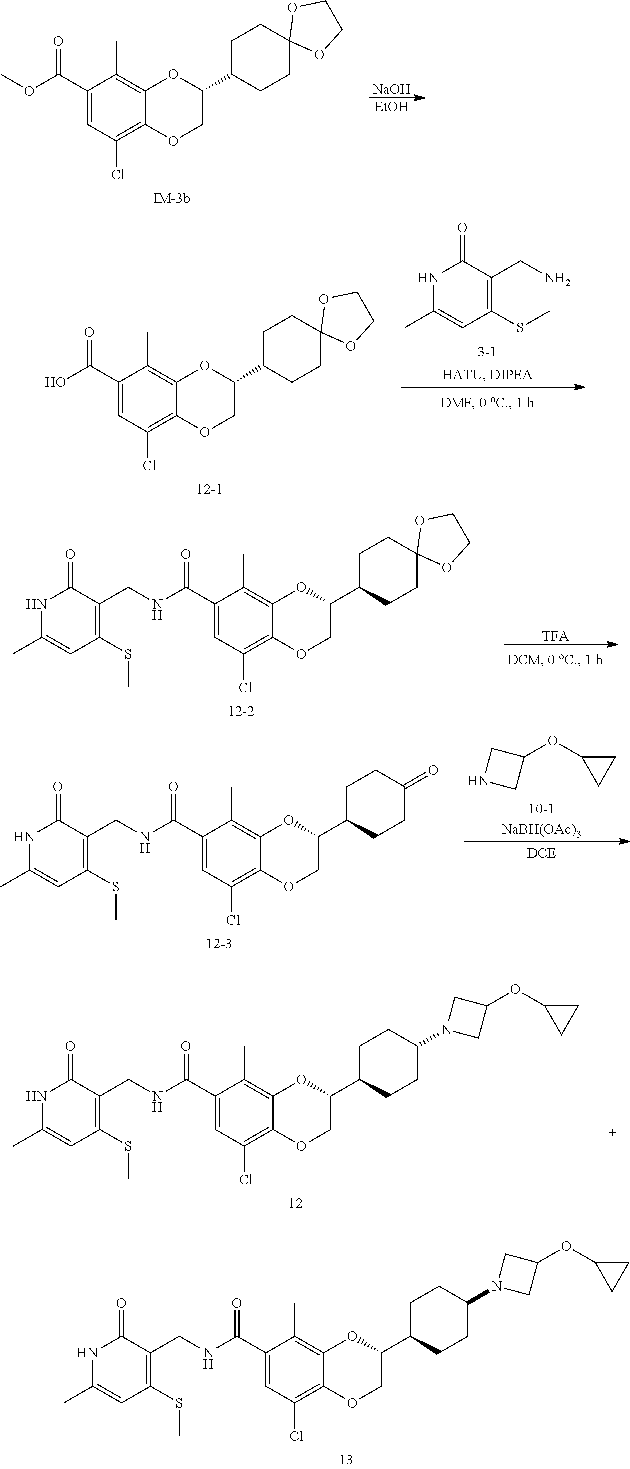

### Step 1, Compound 12-1 Synthesis

Following the synthesis method of Example 6, Step 1, except replacing Step 1, IM-3a with IM-3b (1.5 g, 3.9 mmol), the same synthesis method was applied, to give Compound 12-1 (1.5 g, crude), LCMS (ESI⁺) m/z: 369.1 [M+H]⁺.

### Step 2, Compound 12-2 Synthesis

Following the synthesis method of Example 3, Step 1, except replacing Step 1, 2-3 with 12-1 (250.0 mg, 677.9 mol), the same synthesis method was applied, to give Compound 12-2 (292.0 mg, 545.7 mol), LCMS (ESI⁺) m/z: 535.1 [M+H]⁺.

### Step 3, Compound 12-3 Synthesis

Following the synthesis method of Example 3, Step 2, except replacing Step 2, 3-2 with 12-2 (292.0 mg, 545.7 mol), the same synthesis method was applied, to give Compound 12-3 (250.0 mg, crude), LCMS (ESI⁺) m/z: 491.0 [M+H]⁺.

### Step 4, Compound 12 and 13 Synthesis

Following the synthesis method of Example 6, Step 4, except replacing Step 4, dimethylamine with 10-1 (14.0 mg, 0.1 mmol), the same synthesis method was applied, and further resolved by Prep-MPLC to give Compound 12 (2.0 mg, 0.03 mmol, 20.0% yield) and 13 (2.0 mg, 0.003 mmol, 20.0% yield), LCMS (ESI⁺) m/z: 588.2 [M+H]⁺.

Compound 12 NMR: ¹H NMR (600 MHz, DMSO-d₆) δ 11.52 (s, 1H), 8.01 (t, J=4.6 Hz, 1H), 6.89 (s, 1H), 6.08 (s, 1H), 4.50-4.45 (m, 1H), 4.27-4.25 (m, 2H), 4.07-4.02 (m, 1H), 3.96-3.87 (m, 1H), 3.72-3.66 (m, 2H), 3.27-3.20 (m, 3H), 2.68-2.52 (m, 4H), 2.45 (s, 3H), 2.40-2.30 (m, 2H), 2.16 (s, 3H), 2.13 (s, 3H), 2.20-1.91 (m, 3H), 1.78-1.72 (m, 1H), 1.52-1.60 (m, 1H), 1.25-1.06 (m, 4H).

Compound 13 NMR: ¹H NMR (600 MHz, DMSO-d₆) δ 11.52 (s, 1H), 8.01 (t, J=4.2 Hz, 1H), 6.89 (s, 1H), 6.08 (s, 1H), 4.44-4.41 (m, 1H), 4.27 (d, J=4.8 Hz, 2H), 4.08-4.05 (m, 1H), 3.97-3.93 (m, 1H), 3.88-3.83 (m, 2H), 3.30-3.25 (m, 2H), 2.66 (m, 2H), 2.46 (s, 3H), 2.45 (s, 3H), 2.36 (s, 2H), 2.16 (s, 3H), 2.13 (s, 3H), 1.92-1.88 (m, 2H), 1.70-1.56 (m, 4H), 1.46-1.37 (m, 3H).

### Example 11, Compound 14 and 15 Synthesis

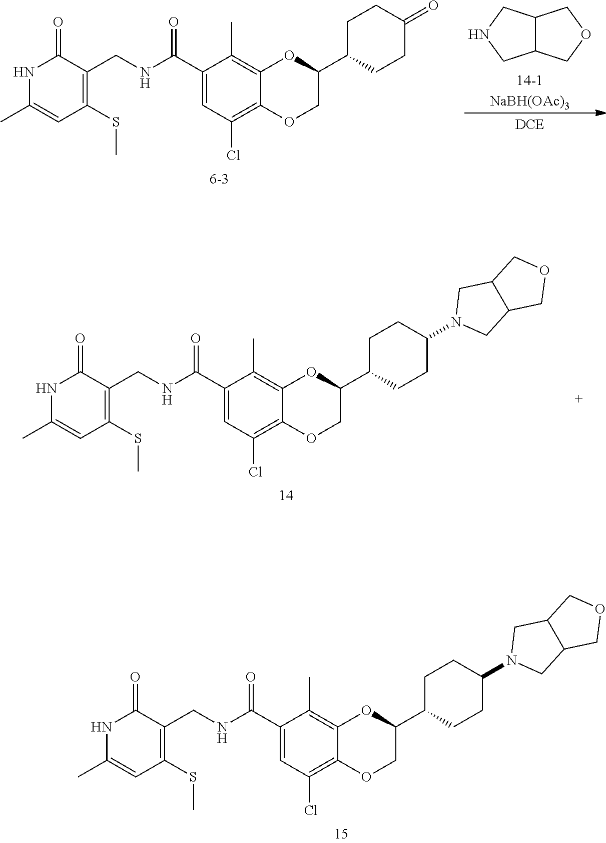

### Step 1, Compound 8 and 9 Synthesis

Following the synthesis method of Example 6, Step 4, except replacing Step 4, dimethylamine with 14-1 (83.0 mg, 733.2 mol), the same synthesis method was applied, and further resolved by Prep-MPLC to give Compound 14 (20.0 mg, 34.0 mol, 8.4% yield) and 15 (20.0 mg, 34.0 mol, 8.4% yield), LCMS (ESI⁺) m/z: 588.2 [M+H]⁺.

Compound 14 NMR: ¹H NMR (400 MHz, DMSO-d₆) δ 11.52 (s, 1H), 8.01 (t, J=4.4 Hz, 1H), 6.90 (s, 1H), 6.08 (s, 1H), 4.46 (dd, J=11.2, 2.0 Hz, 1H), 4.27 (d, J=4.4 Hz, 2H), 4.07-4.02 (m, 1H), 3.92-3.88 (m, 1H), 3.72-3.69 (m, 2H), 3.38-3.34 (m, 2H), 2.67-2.61 (m, 2H), 2.61-2.57 (m, 1H), 2.45 (s, 3H), 2.37-2.31 (m, 2H), 2.16 (s, 3H), 2.14 (s, 3H), 1.99-1.90 (m, 4H), 1.76-1.72 (m, 1H), 1.58-1.54 (m, 1H), 1.22-1.05 (m, 5H).

Compound 15 NMR: ¹H NMR (400 MHz, DMSO-d₆) δ 11.52 (s, 1H), 8.01 (t, J=4.4 Hz, 1H), 6.89 (s, 1H), 6.08 (s, 1H), 4.43 (dd, J=11.2, 2.0 Hz, 1H), 4.27 (d, J=4.4 Hz, 2H), 4.10-4.05 (m, 1H), 3.98-3.94 (m, 1H), 3.87-3.83 (m, 2H), 3.30-3.25 (m, 2H), 2.68-2.64 (s, 2H), 2.45 (s, 3H), 2.37-2.26 (m, 3H), 2.16 (s, 3H), 2.13 (s, 3H), 1.82-1.78 (m, 2H), 1.65-1.59 (m, 5H), 1.43-1.37 (m, 4H).

### Example 12, Compound 16 Synthesis

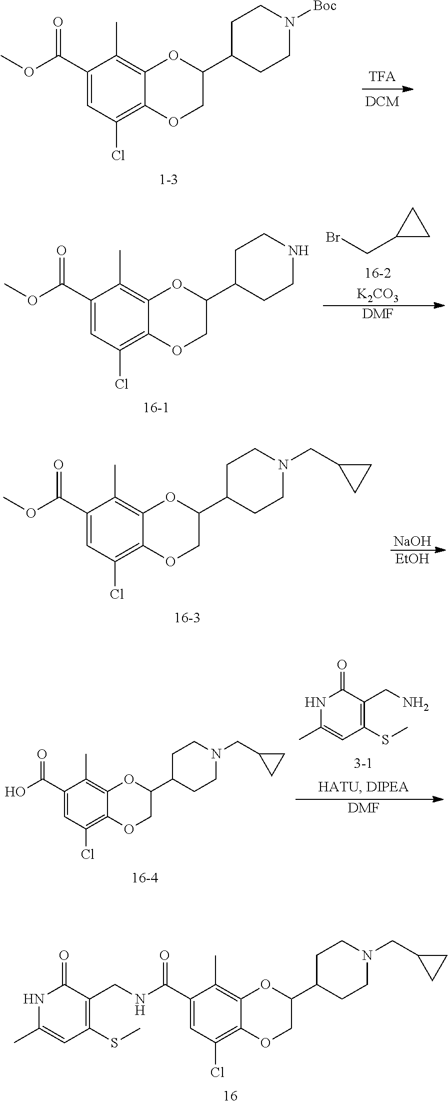

### Step 1, Compound 16-1 Synthesis

Following the synthesis method of Example 1, Step 4, except replacing Step 4, 1-5 with 1-3 (340.0 mg, 798.3 mol), the same synthesis method was applied, to give Compound 16-1 (300.0 mg, crude), LCMS (ESI⁺) m/z: 326.1 [M+H]⁺.

### Step 2, Compound 16-3 Synthesis

A single-necked flask was added with substrate 16-1 (300.0 mg, crude). The mixture was dissolved with DMF (5 mL) under stirring, and then added with 16-2 (147.4 mg, 1.1 mmol), heated to 90° C. to react for 16 hours, with LC-MS monitoring. After the completion of the reaction, the reaction mixture was cooled to room temperature, added with water and ethyl acetate to extract three times. The organic phase was combined, washed with saturated saline solution, and dried with anhydrous sodium sulfate, and then purified by medium pressure liquid chromatography, to give the product 16-3 (247.0 mg, 650.2 mol, 81.4% yield), LCMS (ESI⁺) m/z: 380.2 [M+H]⁺.

### Step 3, Compound 16-4 Synthesis

Following the synthesis method of Example 6, Step 1, except replacing Step 1, IM-3a with 16-3 (247.0 mg, 650.2 mol,), the same synthesis method was applied, to give Compound 16-4 (240.0 mg, crude), LCMS (ESI⁺) m/z: 366.1 [M+H]⁺.

### Step 4, Compound 16 Synthesis

Following the synthesis method of Example 3, Step 1, except replacing Step 1, 2-3 with 16-4 (80.0 mg, 218.6 mol,), the same synthesis method was applied, to give Compound 16 (15.0 mg, 28.2 mol, 12.9% yield), LCMS (ESI⁺) m/z: 532.1 [M+H]⁺.

¹H NMR (400 MHz, DMSO-d₆) δ 11.51 (s, 1H), 8.02 (t, J=4.4 Hz, 1H), 6.90 (s, 1H), 6.08 (s, 1H), 4.48-4.42 (m, 1H), 4.27 (d, J=4.4 Hz, 2H), 4.07 (dd, J=11.4, 7.2 Hz, 1H), 4.08-4.01 (m, 1H), 3.99-3.92 (m, 1H), 3.00 (d, J=11.0 Hz, 2H), 2.45 (s, 3H), 2.16 (s, 3H), 2.14 (s, 3H), 2.13-2.11 (m, 2H), 1.89-1.79 (m, 3H), 1.66-1.51 (m, 2H), 1.48-1.40 (m, 2H), 0.87-0.77 (m, 1H), 0.47-0.40 (m, 2H), 0.06-0.01 (m, 2H).

### Example 13, Compound 17 Synthesis

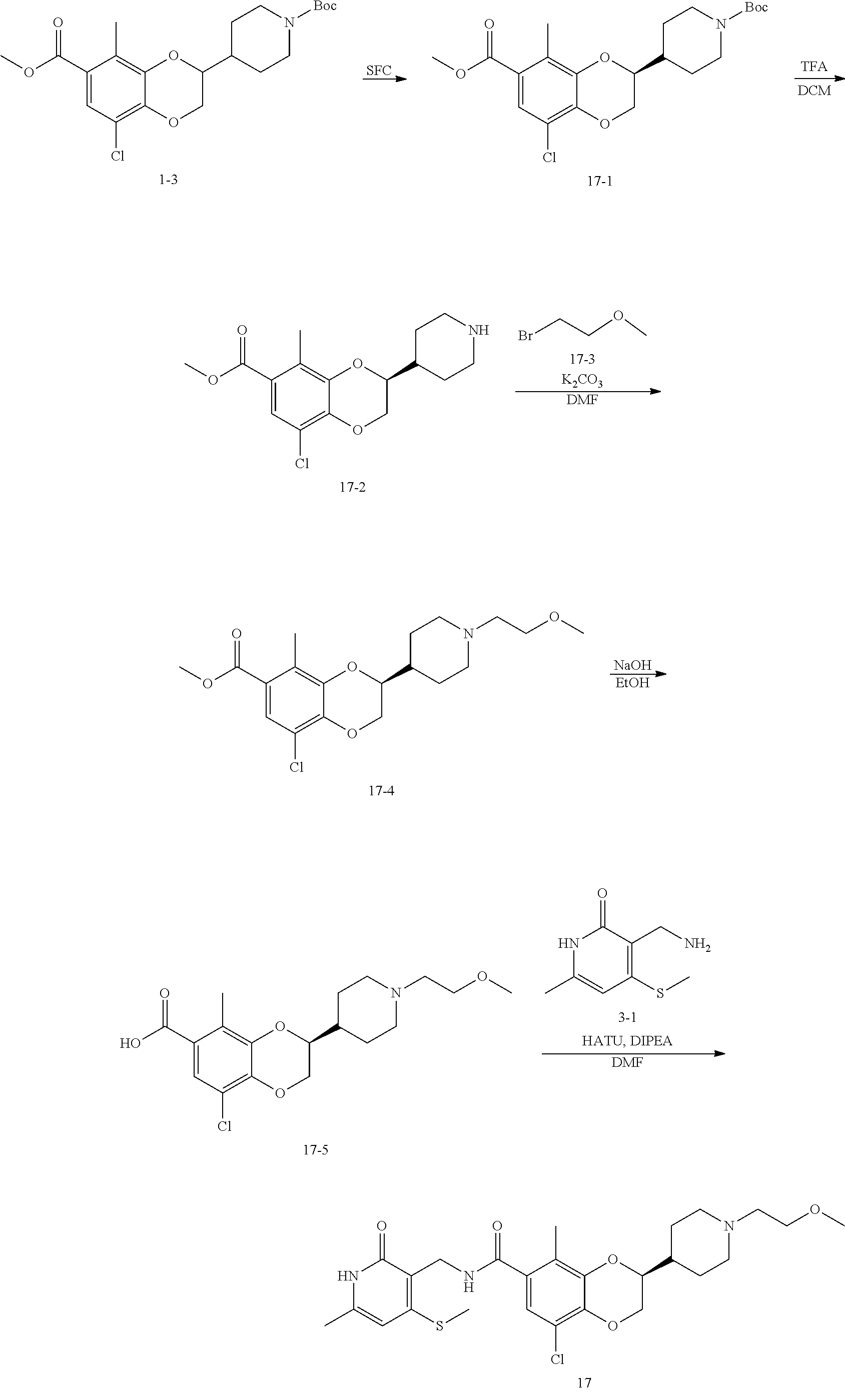

### Step 1, Compound 17-1 Synthesis

Compound 17-1 was obtained by SFC resolution of Compound 1-3.

### Step 2, Compound 17-2 Synthesis

Following the synthesis method of Example 1, Step 4, except replacing Step 4, 1-5 with 17-1 (300.0 mg, 0.7 mmol), the same synthesis method was applied, to give Compound 17-2 (300.0 mg, crude), LCMS (ESI⁺) m/z: 326.1 [M+H]⁺.

### Step 3, Compound 17-4 Synthesis

Following the synthesis method of Example 12, Step 2, except replacing Step 2, 16-1 with 17-2 (100.0 mg, 0.3 mmol), 16-2 with 17-3 (64.0 mg, 0.5 mmol), the same synthesis method was applied, to give Compound 17-4 (132.0 mg, crude), LCMS (ESI⁺) m/z: 384.1 [M+H]⁺.

### Step 4, Compound 17-5 Synthesis

Following the synthesis method of Example 6, Step 1, except replacing Step 1, IM-3a with 17-4 (132.0 mg, crude), the same synthesis method was applied, to give Compound 17-5 (90.0 mg, crude), LCMS (ESI⁺) m/z: 370.1 [M+H]⁺.

### Step 5, Compound 17 Synthesis

Following the synthesis method of Example 3, Step 1, except replacing Step 1, 2-3 with 17-5 (20.0 mg, 54.1 mol), the same synthesis method was applied, to give Compound 17 (5.0 mg, 9.0 mol, 17.2% yield), LCMS (ESI⁺) m/z: 536.6 [M+H]⁺. 1H NMR (400 MHz, DMSO-d₆) δ 11.51 (s, 1H), 8.01 (t, J=4.4 Hz, 1H), 6.90 (s, 1H), 6.08 (s, 1H), 4.45 (dd, J=11.4, 2.0 Hz, 1H), 4.27 (d, J=4.4 Hz, 2H), 4.07-4.05 (m, 1H), 3.97-3.95 (m, 1H), 3.41 (t, J=5.8 Hz, 2H), 3.22 (s, 3H), 2.90 (d, J=10.8 Hz, 2H), 2.45 (s, 3H), 2.44-2.41 (m, 2H), 2.16 (s, 3H), 2.14 (s, 3H), 1.96-1.78 (m, 3H), 1.66-1.51 (m, 2H), 1.46-1.34 (m, 2H).

### Example 14, Compound 18 and 19 Synthesis

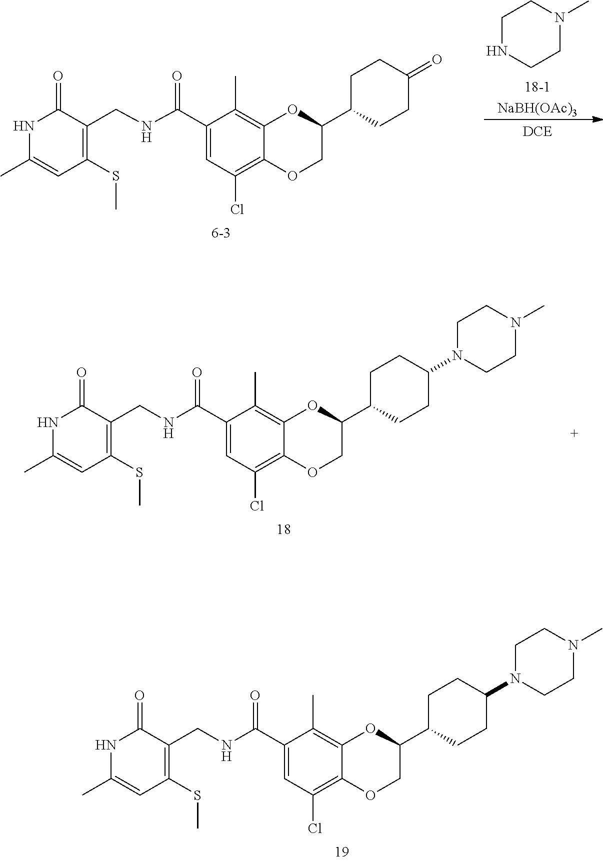

### Step 1, Compound 18 and 19 Synthesis

Following the synthesis method of Example 6, Step 4, except replacing Step 4, dimethylamine with 18-1 (17.0 mg, 0.2 mmol), the same synthesis method was applied, and further resolved by Prep-MPLC to give Compound 18 (15.0 mg, 0.03 mmol, 18.3% yield) and 19 (20.0 mg, 0.04 mmol, 24.0% yield), LCMS (ESI⁺) m/z: 575.2 [M+H]⁺.

Compound 18 NMR: ¹H NMR (400 MHz, DMSO-d₆) δ 11.52 (s, 1H), 8.01 (t, J=4.4 Hz, 1H), 6.90 (s, 1H), 6.08 (s, 1H), 4.47 (d, J=11.2 Hz, 1H), 4.27 (d, J=4.4 Hz, 2H), 4.08-4.06 (m, 1H), 3.92-3.90 (m, 1H), 2.49-2.42 (m, 8H), 2.33-2.31 (m, 4H), 2.16 (s, 3H), 2.14 (s, 3H), 2.13 (s, 3H), 1.85-1.78 (m, 4H), 1.57-1.48 (m, 2H), 1.47-1.36 (m, 3H).

Compound 19 NMR: ¹H NMR (400 MHz, DMSO-d₆) δ 11.52 (s, 1H), 8.01 (t, J=4.4 Hz, 1H), 6.90 (s, 1H), 6.08 (s, 1H), 4.47 (d, J=11.2 Hz, 1H), 4.29 (d, J=4.4 Hz, 2H), 4.08-4.03 (m, 1H), 3.95-3.88 (m, 1H), 2.47-2.34 (m, 8H), 2.35-2.23 (m, 4H), 2.16 (s, 3H), 2.14 (s, 3H), 2.12 (s, 3H), 2.04-1.98 (m, 1H), 1.90-1.76 (m, 3H), 1.58-1.49 (m, 1H), 1.23-1.11 (m, 4H).

### Example 15, Compound 20 and 21 Synthesis

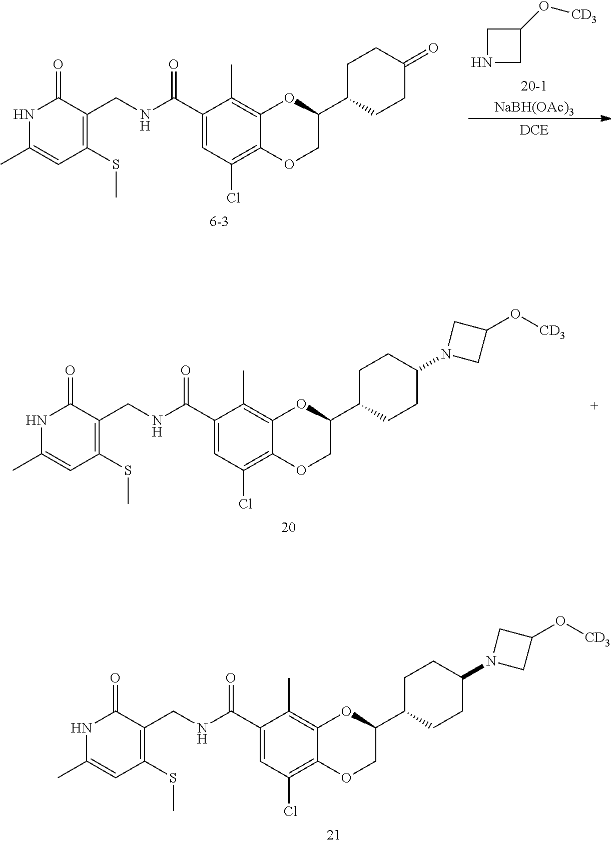

### Step 1, Compound 20 and 21 Synthesis

Following the synthesis method of Example 6, Step 4, except replacing Step 4, dimethylamine with 20-1 (20.0 mg, 0.2 mmol), the same synthesis method was applied, and further resolved by Prep-MPLC to give Compound 20 (5.0 mg, 9.0 mol, 8.7% yield) and 21 (9.0 mg, 0.02 mmol, 16.0% yield), LCMS (ESI⁺) m/z: 565.2 [M+H]⁺.

Compound 20 NMR: ¹H NMR (400 MHz, Methanol-d₄) δ 6.94 (s, 1H), 6.27 (s, 1H), 4.49 (s, 2H), 4.44-4.40 (m, 1H), 4.09-3.99 (m, 2H), 3.90-3.86 (m, 1H), 3.65-3.61 (m, 2H), 3.05-3.01 (m, 2H), 2.52 (s, 3H), 2.29 (s, 3H), 2.20 (s, 3H), 2.15-2.10 (m, 1H), 2.05-2.00 (m, 2H), 1.94-1.90 (m, 2H), 1.85-1.81 (m, 1H), 1.63-1.58 (m, 2H), 1.05-0.98 (m, 2H).

Compound 21 NMR: ¹H NMR (400 MHz, Methanol-d₄) δ 6.94 (s, 1H), 6.27 (s, 1H), 4.49 (s, 2H), 4.46-4.42 (m, 1H), 4.09-3.98 (m, 2H), 3.95-3.86 (m, 1H), 3.64-3.60 (m, 2H), 3.10-2.95 (m, 2H), 2.52 (s, 3H), 2.30 (s, 3H), 2.20 (s, 3H), 2.14-2.09 (m, 1H), 2.07-1.94 (m, 2H), 1.96-1.88 (m, 2H), 1.87-1.76 (m, 1H), 1.70-1.50 (m, 2H), 1.09-0.96 (m, 2H).

### Example 16, Compound 22 and 23 Synthesis

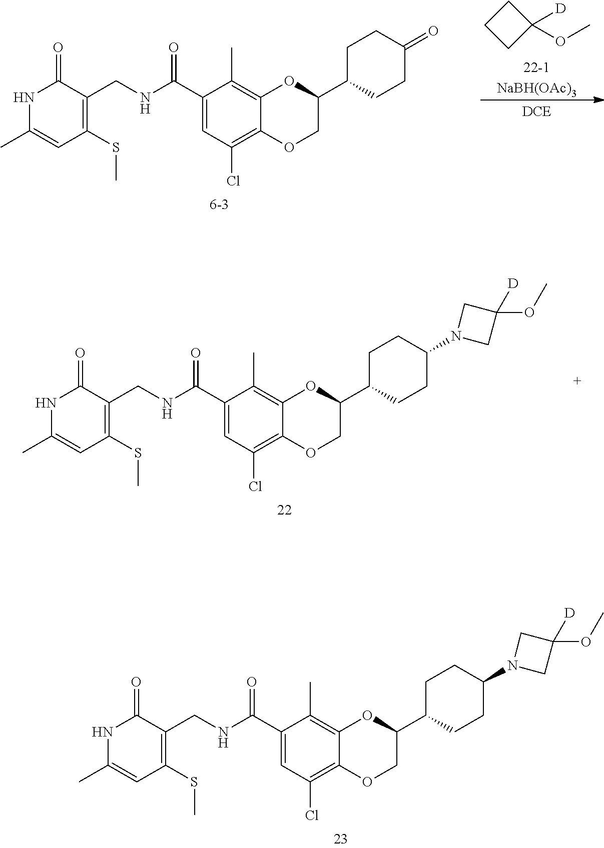

### Step 1, Compound 22 and 23 Synthesis

Following the synthesis method of Example 6, Step 4, except replacing Step 4, dimethylamine with 22-1 (48.0 mg, 0.5 mmol), the same synthesis method was applied, and further resolved by Prep-MPLC to give Compound 22 (15.0 mg, 0.03 mmol, 25.0% yield) and 23 (15.0 mg, 0.03 mmol, 25.0% yield), LCMS (ESI⁺) m/z: 563.3 [M+H]⁺.

Compound 22 NMR: ¹H NMR (600 MHz, DMSO-d₆) δ 11.51 (s, 1H), 8.02 (t, J=4.2 Hz, 1H), 6.90 (s, 1H), 6.08 (s, 1H), 4.45-4.44 (m, 1H), 4.27 (d, J=4.8 Hz, 2H), 4.06-4.03 (m, 1H), 3.92-3.89 (m, 1H), 3.43 (d, J=7.2 Hz, 2H), 3.13 (s, 3H), 2.71 (d, J=7.8 Hz, 2H), 2.45 (s, 3H), 2.16 (s, 3H), 2.14 (s, 3H), 1.96-1.93 (m, 1H), 1.89-1.85 (m, 1H), 1.77-1.71 (m, 3H), 1.54-1.52 (m, 1H), 1.18-1.14 (m, 2H), 0.88-0.84 (m, 2H).

Compound 23 NMR: ¹H NMR (600 MHz, DMSO-d₆) δ 11.52 (s, 1H), 8.02 (t, J=4.2 Hz, 1H), 6.89 (s, 1H), 6.08 (s, 1H), 4.43 (d, J=9.6 Hz, 1H), 4.27 (d, J=4.2 Hz, 2H), 4.07-4.04 (m, 1H), 3.94-3.92 (m, 1H), 3.45 (t, J=4.8 Hz, 2H), 3.14 (s, 3H), 2.67-2.66 (m, 2H), 2.45 (s, 3H), 2.22-2.20 (m, 1H), 2.16 (s, 3H), 2.13 (s, 3H), 1.62-1.28 (m, 9H).

### Example 17, Compound 24 Synthesis

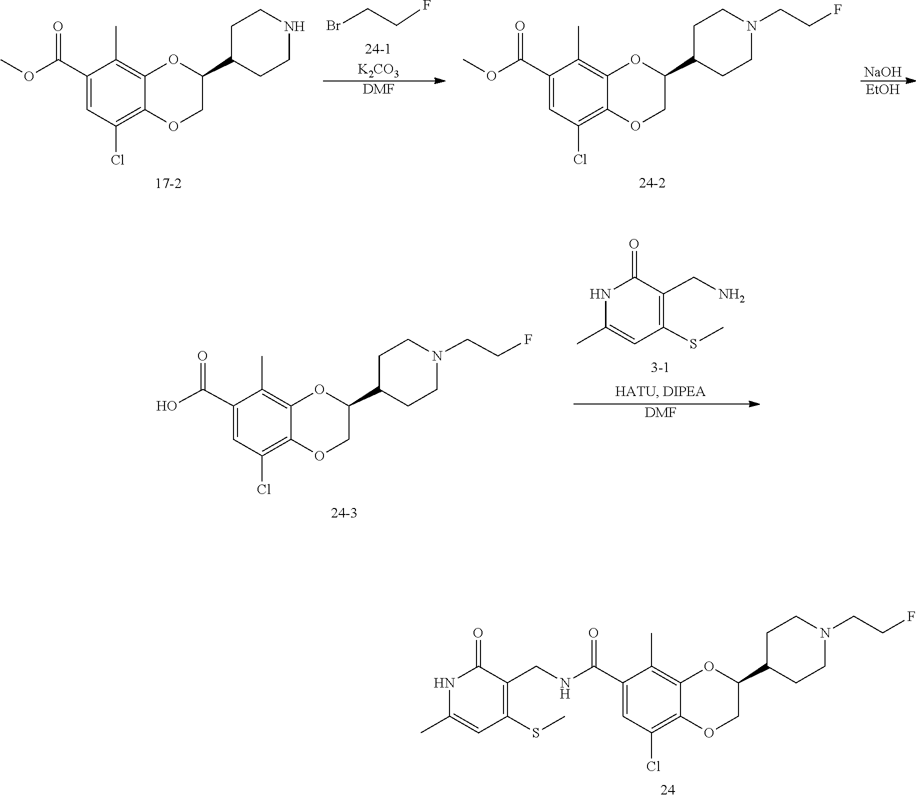

### Step 1, Compound 24-2 Synthesis

Following the synthesis method of Example 12, Step 2, except replacing Step 2, 16-1 with 17-2 (100.0 mg, 0.3 mmol), 16-2 with 24-1 (58.0 mg, 0.5 mmol), the same synthesis method was applied, to give Compound 24-2 (100.0 mg, crude), LCMS (ESI⁺) m/z: 372.1 [M+H]⁺.

### Step 2, Compound 24-3 Synthesis

Following the synthesis method of Example 6, Step 1, except replacing Step 1, IM-3a with 24-2 (100.0 mg, crude), the same synthesis method was applied, to give Compound 24-3 (50.0 mg, crude), LCMS (ESI⁺) m/z: 358.1 [M+H]⁺.

### Step 3, Compound 24 Synthesis

Following the synthesis method of Example 3, Step 1, except replacing Step 1, 2-3 with 24-3 (30.0 mg, 0.08 mmol), the same synthesis method was applied, to give Compound 24 (1.3 mg, 2.5 mol, 3.0% yield), LCMS (ESI⁺) m/z: 524.5 [M+H]⁺.

¹H NMR (400 MHz, DMSO-d₆) δ 11.52 (s, 1H), 8.01 (t, J=4.4 Hz, 1H), 6.90 (s, 1H), 6.08 (s, 1H), 4.57 (d, J=4.8 Hz, 1H), 4.49-4.41 (m, 2H), 4.27 (d, J=4.4 Hz, 2H), 4.07-4.03 (m, 1H), 3.99-3.96 (m, 1H), 2.95-2.91 (m, 2H), 2.63-2.61 (m, 1H), 2.45 (s, 3H), 2.18 (s, 3H), 2.14 (s, 3H), 2.02-1.80 (m, 2H), 1.86-1.80 (m, 2H), 1.67-1.54 (m, 2H), 1.47-1.38 (m, 2H).

### Example 18, Compound 25 Synthesis

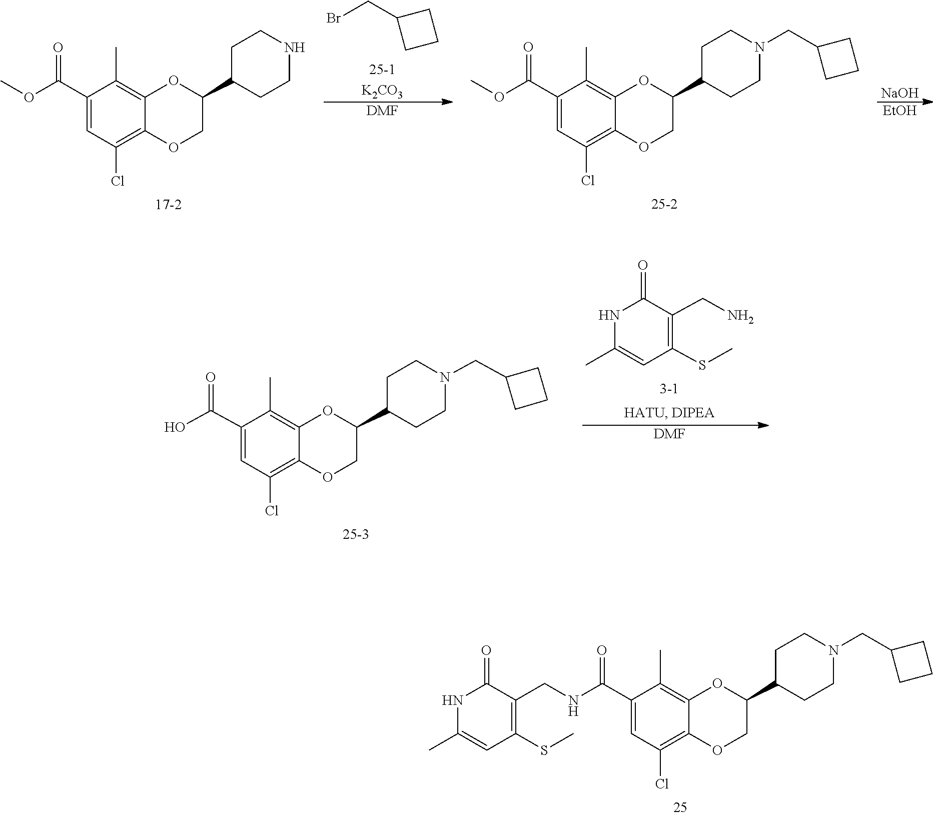

### Step 1, Compound 25-2 Synthesis

Following the synthesis method of Example 12, Step 2, except replacing Step 2, 16-1 with 17-2 (100.0 mg, 0.3 mmol), 16-2 with 25-1 (68.0 mg, 0.5 mmol), the same synthesis method was applied, to give Compound 25-2 (100.0 mg, crude), LCMS (ESI⁺) m/z: 394.1 [M+H]⁺.

### Step 2, Compound 25-3 Synthesis

Following the synthesis method of Example 6, Step 1, except replacing Step 1, IM-3a with 25-2 (100.0 mg, crude), the same synthesis method was applied, to give Compound 25-3 (50.0 mg, crude), LCMS (ESI⁺) m/z: 380.1 [M+H]⁺.

### Step 3, Compound 25 Synthesis

Following the synthesis method of Example 3, Step 1, except replacing Step 1, 2-3 with 25-3 (25.0 mg, 0.07 mmol), the same synthesis method was applied, to give Compound 25 (3.5 mg, 6.4 mol, 9.7% yield), LCMS (ESI⁺) m/z: 546.6 [M+H]⁺.

¹H NMR (400 MHz, DMSO-d₆) δ 11.52 (s, 1H), 8.02 (t, J=4.8 Hz, 1H), 6.90 (s, 1H), 6.08 (s, 1H), 4.46-4.42 (m, 1H), 4.27 (d, J=4.4 Hz, 2H), 4.08-4.03 (m, 1H), 3.96-3.93 (m, 1H), 2.84-2.81 (m, 2H), 2.45 (s, 3H), 2.30-2.28 (m, 2H), 2.16 (s, 3H), 2.15-2.13 (m, 3H), 2.01-1.97 (m, 2H), 1.84-1.77 (m, 5H), 1.63-1.58 (m, 5H), 1.39-1.36 (m, 2H).

### Example 19, Compound 26 and 27 Synthesis

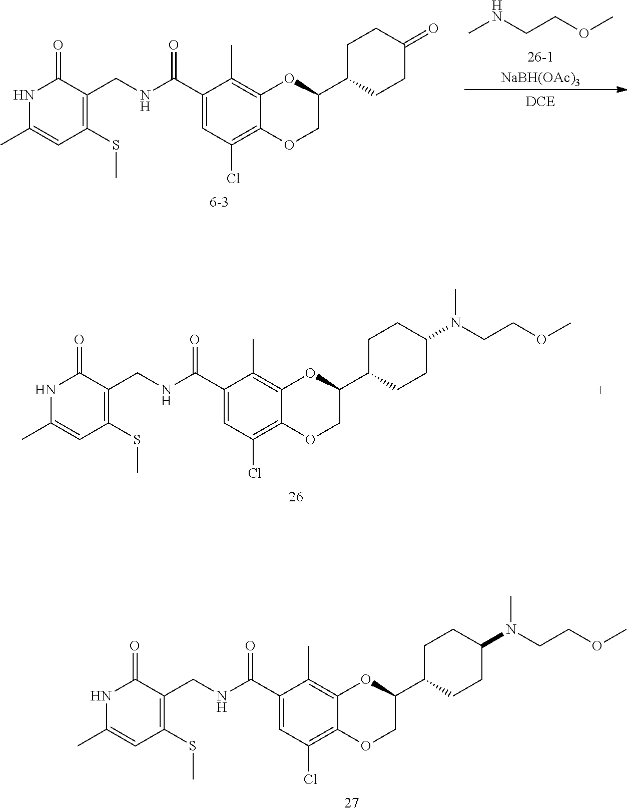

### Step 1, Compound 26 and 27 Synthesis

Following the synthesis method of Example 6, Step 4, except replacing Step 4, dimethylamine with 26-1 (17.0 mg, 0.2 mmol), the same synthesis method was applied, and further resolved by Prep-MPLC to give Compound 26 (15.0 mg, 0.03 mmol, 28.0% yield) and 27 (15.0 mg, 0.03 mmol, 28.0% yield), LCMS (ESI⁺) m/z: 564.2 [M+H]⁺.

Compound 26 NMR: ¹H NMR (600 MHz, DMSO-d₆) δ 11.52 (s, 1H), 8.02 (t, J=4.2 Hz, 1H), 6.90 (s, 1H), 6.08 (s, 1H), 4.47-4.45 (m, 1H), 4.27 (d, J=4.8 Hz, 2H), 4.18-4.15 (m, 1H), 4.08-4.05 (m, 1H), 3.39 (t, J=6.0 Hz, 2H), 3.22 (s, 3H), 2.57 (t, J=6.6 Hz, 2H), 2.45 (s, 3H), 2.37-2.33 (m, 1H), 2.19 (s, 3H), 2.16 (s, 3H), 2.14 (s, 3H), 1.84-1.79 (m, 1H), 1.75-1.63 (m, 4H), 1.52-1.44 (m, 4H).

Compound 27 NMR: ¹H NMR (600 MHz, DMSO-d₆) δ 11.52 (s, 1H), 8.02 (t, J=4.2 Hz, 1H), 6.90 (s, 1H), 6.08 (s, 1H), 4.45-4.43 (m, 1H), 4.27 (d, J=4.2 Hz, 2H), 4.07-4.04 (m, 1H), 3.92-3.90 (m, 1H), 3.39-3.37 (m, 2H), 3.30-3.29 (m, 1H), 3.18 (s, 3H), 2.63-2.61 (m, 1H), 2.45 (s, 3H), 2.24-2.22 (m, 3H), 2.16-2.14 (m, 7H), 2.03-2.01 (m, 1H), 1.80-1.79 (m, 3H), 1.55-1.54 (m, 1H), 1.26-1.17 (m, 4H).

### Example 20, Compound 28 and 29 Synthesis

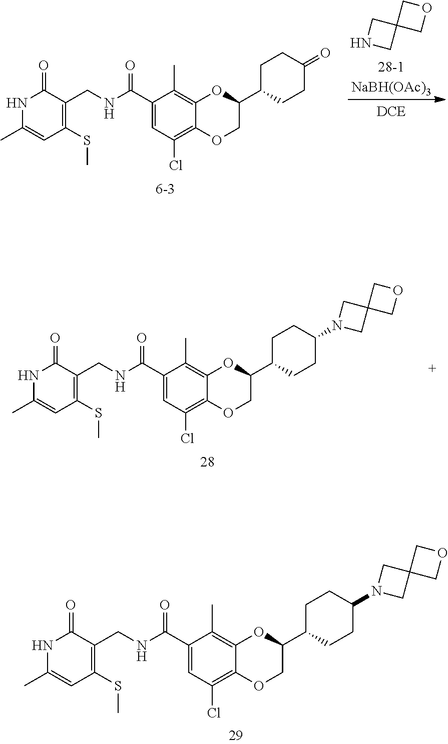

### Step 1, Compound 28 and 29 Synthesis

Following the synthesis method of Example 6, Step 4, except replacing Step 4, dimethylamine with 28-1 (20.0 mg, 0.2 mmol), the same synthesis method was applied, and further resolved by Prep-MPLC to give Compound 28 (1.1 mg, 2.0 mol, 1.9% yield) and 29 (1.2 mg, 2.0 mol, 1.9% yield), LCMS (ESI⁺) m/z: 574.1 [M+H]⁺.

Compound 28 NMR: ¹H NMR (400 MHz, DMSO-d₆) δ 11.52 (s, 1H), 8.02 (t, J=4.4 Hz, 1H), 6.90 (s, 1H), 6.08 (s, 1H), 4.59 (s, 4H), 4.45-4.43 (m, 1H), 4.27 (d, J=4.4 Hz, 2H), 4.07-4.02 (m, 1H), 3.93-3.90 (m, 1H), 3.29 (s, 4H), 2.45 (s, 3H), 2.16 (s, 3H), 2.13 (s, 3H), 1.97-1.91 (m, 1H), 1.75-1.74 (m, 3H), 1.55-1.53 (m, 1H), 1.24-1.09 (m, 3H), 0.89-0.80 (m, 2H).

Compound 29 NMR: ¹H NMR (400 MHz, DMSO-d₆) δ 11.51 (s, 1H), 8.02 (t, J=4.4 Hz, 1H), 6.89 (s, 1H), 6.08 (s, 1H), 4.59 (s, 4H), 4.42-4.40 (m, 1H), 4.27 (d, J=4.4 Hz, 2H), 4.07-4.02 (m, 1H), 3.96-3.92 (m, 1H), 3.19 (s, 4H), 2.45 (s, 3H), 2.16 (s, 3H), 2.13 (s, 3H), 1.58-1.49 (m, 5H), 1.47-1.24 (m, 5H).

### Example 21, Compound 30 and 31 Synthesis

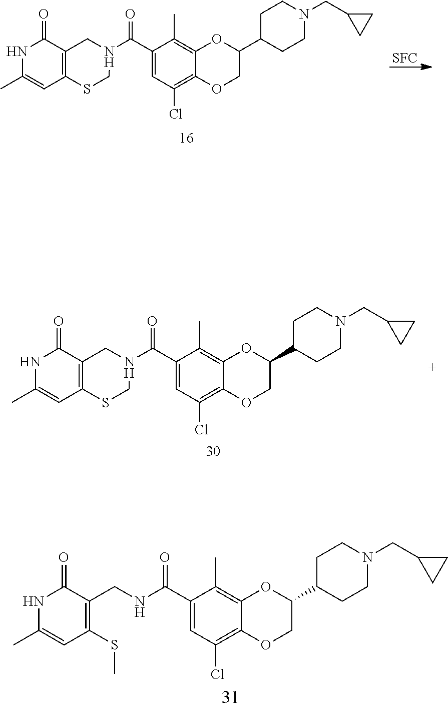

### Step 1, Compound 25-2 Synthesis

Compound 30 and 31 was obtained by SFC resolution of Compound 16.

LCMS (ESI⁺) m/z: 532.1 [M+H]⁺

Compound 30 NMR: ¹H NMR (400 MHz, DMSO-d₆) δ 11.52 (s, 1H), 8.02 (t, J=4.4 Hz, 1H), 6.90 (s, 1H), 6.08 (s, 1H), 4.48-4.42 (m, 1H), 4.27 (d, J=4.4 Hz, 2H), 4.10-4.05 (m, 1H), 3.99-3.92 (m, 1H), 3.00 (d, J=11.0 Hz, 2H), 2.45 (s, 3H), 2.17-2.15 (m, 3H), 2.15-2.13 (m, 3H), 2.12-2.10 (m, 1H), 1.90-1.88 (m, 1H), 1.89-1.70 (m, 3H), 1.68-1.62 (m, 1H), 1.60-1.52 (m, 1H), 1.48-1.39 (m, 2H), 0.83-0.78 (m, 1H), 0.48-0.42 (m, 2H), 0.08-0.01 (m, 2H).

Compound 31 NMR: ¹H NMR (400 MHz, DMSO-d₆) δ 11.52 (s, 1H), 8.03 (t, J=4.4 Hz, 1H), 6.92 (s, 1H), 6.08 (s, 1H), 4.49-4.47 (m, 1H), 4.27 (d, J=4.4 Hz, 2H), 4.12-4.08 (m, 1H), 4.00-3.98 (m, 1H), 2.96-2.89 (m, 1H), 2.45 (s, 3H), 2.17 (s, 3H), 2.17-2.15 (m, 6H), 2.03-1.89 (m, 2H), 1.80-1.68 (m, 2H), 1.60-1.46 (m, 2H), 1.25-1.21 (m, 3H), 0.95-0.80 (m, 2H), 0.58-0.45 (m, 2H), 0.25-0.10 (m, 2H).

### Example 22, Compound 32 and 33 Synthesis

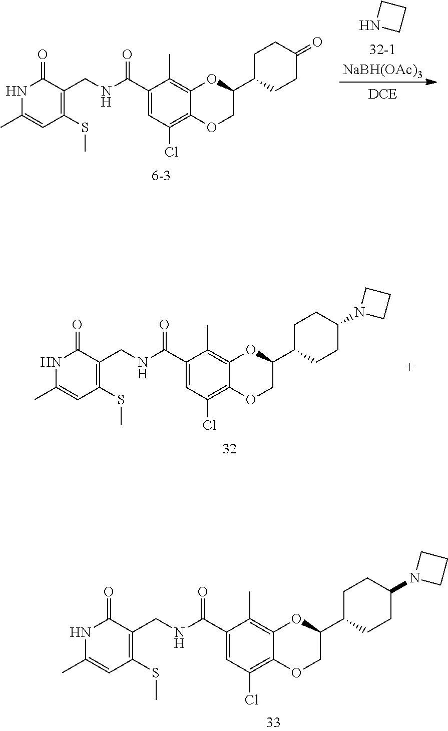

### Step 1, Compound 32 and 33 Synthesis

Following the synthesis method of Example 6, Step 4, except replacing Step 4, dimethylamine with 32-1 (11.0 mg, 0.2 mmol), the same synthesis method was applied, and further resolved by Prep-MPLC to give Compound 32 (11.0 mg, 0.02 mmol, 22.0% yield) and 33 (5.0 mg, 9.0 mol, 9.0% yield), LCMS (ESI⁺) m/z: 532.1 [M+H]⁺.

Compound 32 NMR: ¹H NMR (600 MHz, DMSO-d₆) δ 11.52 (s, 1H), 8.01 (t, J=4.2 Hz 1H), 6.89 (s, 1H), 6.08 (s, 1H), 4.44-4.40 (m, 1H), 4.27 (d, J=4.2 Hz, 2H), 4.06-4.04 (m, 1H), 3.96-3.90 (m, 1H), 3.02 (t, J=6.0 Hz, 4H), 2.45 (s, 3H), 2.16 (s, 3H), 2.13 (s, 3H), 1.95-1.91 (m 1H), 1.89-1.83 (m, 2H), 1.76-1.69 (m, 2H), 1.55-1.49 (m, 1H), 1.26-1.12 (m, 4H), 0.89-0.80 (m, 2H).

Compound 33 NMR: ¹H NMR (600 MHz, DMSO-d₆) δ 11.52 (s, 1H), 8.01 (t, J=4.2 Hz, 1H), 6.90 (s, 1H), 6.08 (s, 1H), 4.46-4.43 (m, 1H), 4.27 (d, J=4.2 Hz, 2H), 4.07-4.03 (m, 1H), 3.93-3.90 (m, 1H), 3.05 (t, J=6.0 Hz, 4H), 2.45 (s, 3H), 2.16 (s, 3H), 2.14 (s, 3H), 1.96-1.92 (m, 1H), 1.88 (t, J=6.8 Hz, 2H), 1.74-1.69 (m, 2H), 1.56-1.50 (m, 1H), 1.25-1.11 (m, 4H), 0.85-0.81 (m, 2H).

### Example 23, Compound 34 and 35 Synthesis

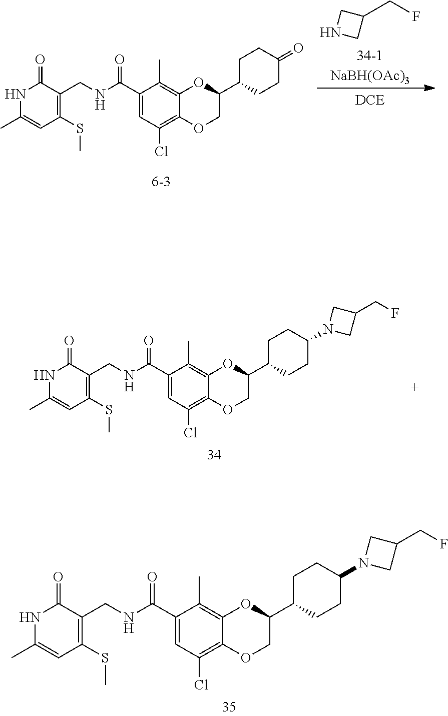

### Step 1, Compound 34 and 35 Synthesis

Following the synthesis method of Example 6, Step 4, except replacing Step 4, dimethylamine with 34-1 (17.0 mg, 0.2 mmol), the same synthesis method was applied, and further resolved by Prep-MPLC to give Compound 34 (18.0 mg, 0.03 mmol, 29.0% yield) and 35 (18.0 mg, 0.03 mmol, 29.0% yield), LCMS (ESI⁺) m/z: 564.3 [M+H]⁺.

### Example 24, Compound 36 and 37 Synthesis

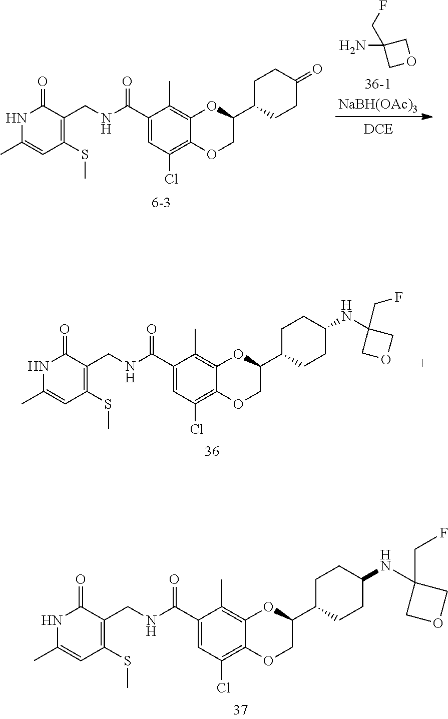

### Step 1, Compound 36 and 37 Synthesis

Following the synthesis method of Example 6, Step 4, except replacing Step 4, dimethylamine with 36-1 (18.0 mg, 0.2 mmol), the same synthesis method was applied, and further resolved by Prep-MPLC to give Compound 36 (18.0 mg, 0.03 mmol, 31.0% yield) and 37 (18.0 mg, 0.03 mmol, 31.0% yield), LCMS (ESI⁺) m/z: 580.2 [M+H]⁺.

### Example 25, Compound 38 Synthesis

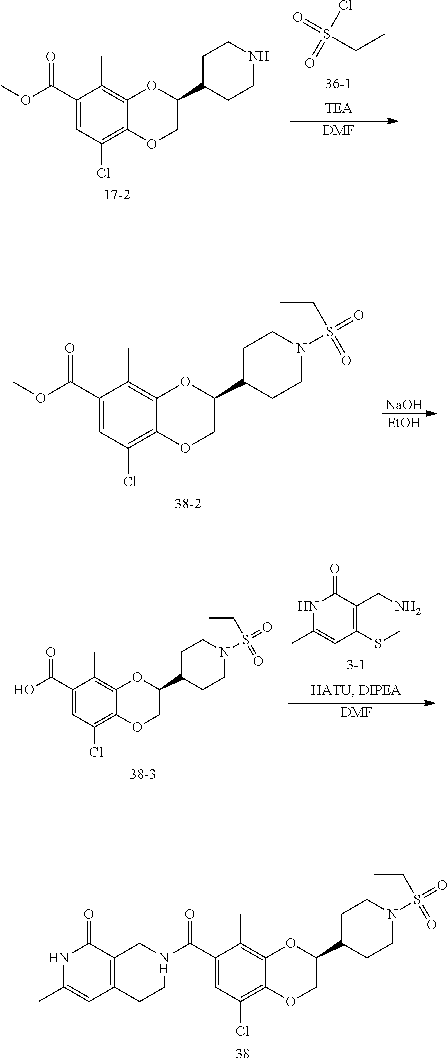

### Step 1, Compound 38-2 Synthesis

A single-necked flask was added with substrate 17-2 (25.0 mg, 0.08 mmol). The mixture was dissolved with DCM (5 mL) under stirring, and then at 0° C. added with TEA (23.0 mg, 0.2 mmol), added with 38-1 (10.3 mg, 0.08 mmol), 0° C. to react for 1 hour, with LC-MS monitoring. After the completion of the reaction, condensated under reduced pressure to give the crude product 38-2 (28.0 mg, crude), LCMS (ESI⁺) m/z: 418.1 [M+H]⁺.

### Step 2, Compound 38-3 Synthesis

Following the synthesis method of Example 6, Step 1, except replacing Step 1, IM-3a with 38-2 (28.0 mg, crude), the same synthesis method was applied, to give Compound 38-3 (25.0 mg, crude), LCMS (ESI⁺) m/z: 404.1 [M+H]⁺.

### Step 3, Compound 38 Synthesis

Following the synthesis method of Example 3, Step 1, except replacing Step 1, 2-3 with 38-3 (25 mg, crude), the same synthesis method was applied, to give Compound 38 (21.0 mg, 0.037 mmol, 47.8% yield), LCMS (ESI⁺) m/z: 570.5 [M+H]⁺.

¹H NMR (600 MHz, DMSO-d₆) δ 11.52 (s, 1H), 8.02 (t, J=4.4 Hz, 1H), 6.91 (s, 1H), 6.08 (s, 1H), 4.46 (d, J=10.2 Hz, 1H), 4.27 (d, J=4.2 Hz, 2H), 4.15-4.07 (m, 1H), 4.06-4.03 (m, 1H), 3.64 (d, J=11.8 Hz, 2H), 3.02 (q, J=7.4 Hz, 2H), 2.84-2.74 (m, 2H), 2.45 (s, 3H), 2.16 (s, 3H), 2.15 (s, 3H), 1.98-1.93 (m, 1H), 1.83-1.73 (m, 2H), 1.48-1.41 (m, 2H), 1.20 (t, J=7.4 Hz, 3H).

### Example 26, Compound 39 and 40 Synthesis

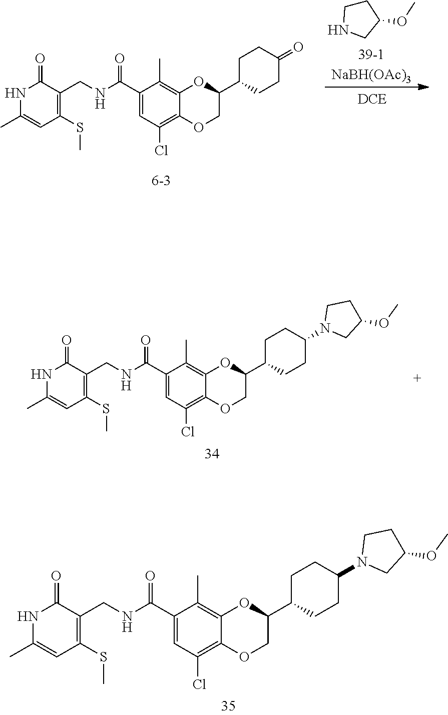

### Step 1, Compound 39 and 40 Synthesis

Following the synthesis method of Example 6, Step 4, except replacing Step 4, dimethylamine with 39-1 (12.0 mg, 0.1 mmol), the same synthesis method was applied, and further resolved by Prep-MPLC to give Compound 39 (10.0 mg, 0.017 mmol, 17.0% yield) and 40 (10.0 mg, 0.017 mmol, 17.0% yield), LCMS (ESI⁺) m/z: 576.3 [M+H]⁺.

Compound 39 NMR: ¹H NMR (600 MHz, Methanol-d₄) δ 6.94 (s, 1H), 6.27 (s, 1H), 4.49 (s, 2H), 4.41-4.38 (m, 1H), 4.09-4.05 (m, 1H), 3.98-3.94 (s, 1H), 3.89-3.86 (t, J=7.2 Hz, 1H), 3.28 (s, 3H), 2.88-2.80 (m, 2H), 2.80-2.75 (m, 1H), 2.63-2.59 (s, 1H), 2.52 (s, 3H), 2.29 (s, 3H), 2.20 (s, 3H), 2.14-2.02 (m, 5H), 1.86-1.80 (m, 2H), 1.68-1.62 (m, 1H), 1.32-1.20 (m, 4H).

Compound 40 NMR: ¹H NMR (600 MHz, Methanol-d₄) δ 6.95 (s, 1H), 6.27 (s, 1H), 4.49 (s, 2H), 4.47-4.44 (m, 1H), 4.13-4.10 (t, J=7.8 Hz, 1H), 4.06-4.03 (m, 1H), 3.96 (s, 1H), 3.28 (s, 3H), 2.82-2.74 (m, 2H), 2.71-2.66 (s, 1H), 2.58-2.55 (m, 1H), 2.52 (s, 3H), 2.29 (s, 3H), 2.20 (s, 3H), 2.10-2.06 (m, 1H), 1.98-1.92 (m, 1H), 1.85-1.77 (m, 2H), 1.76-1.63 (m, 7H), 1.58-1.52 (m, 1H).

### Example 27, Compound 41 and 42 Synthesis

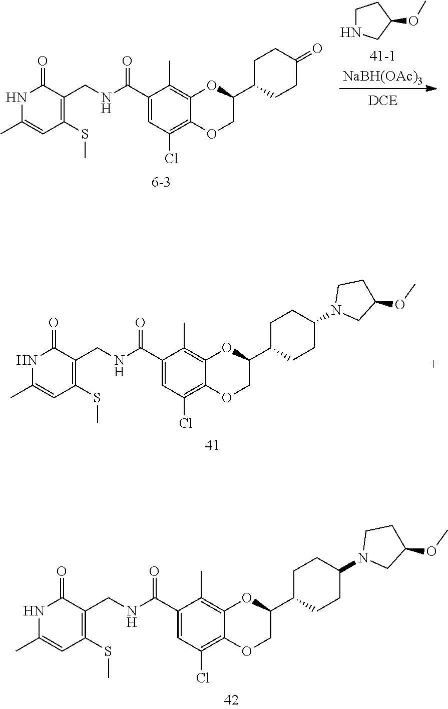

### Step 1, Compound 41 and 42 Synthesis

Following the synthesis method of Example 6, Step 4, except replacing Step 4, dimethylamine with 41-1 (11.0 mg, 0.1 mmol), the same synthesis method was applied, and further resolved by Prep-MPLC to give Compound 41 (14.0 mg, 0.02 mmol, 24.0% yield) and 42 (6.5 mg, 0.01 mmol, 11.0% yield), LCMS (ESI⁺) m/z: 576.3 [M+H]⁺.

Compound 41 NMR: ¹H NMR (600 MHz, Methanol-d₄) δ 6.94 (s, 1H), 6.27 (s, 1H), 4.49 (s, 2H), 4.42 (dd, J=11.4, 1.8 Hz, 1H), 4.07-4.04 (m, 1H), 3.96-3.94 (m, 1H), 3.89 (t, J=7.2 Hz, 1H), 3.28 (s, 3H), 2.86-2.84 (m, 2H), 2.77-2.75 (m, 1H), 2.63-2.61 (m, 1H), 2.52 (s, 3H), 2.29 (s, 3H), 2.20 (s, 3H), 2.12-2.06 (m, 5H), 1.86-1.81 (m, 2H), 1.65-1.64 (m, 1H), 1.30-1.25 (m, 4H).

Compound 42 NMR: ¹H NMR (600 MHz, Methanol-d₄) δ 6.95 (s, 1H), 6.27 (s, 1H), 4.50 (s, 2H), 4.46 (dd, J=11.4, 1.8 Hz, 1H), 4.15-4.09 (m, 1H), 4.05-4.01 (m, 1H), 3.96 (s, 1H), 3.28 (s, 3H), 2.85-2.74 (m, 2H), 2.74-2.65 (m, 1H), 2.58-2.53 (m, 1H), 2.52 (s, 3H), 2.29 (s, 3H), 2.20 (s, 3H), 2.12-2.04 (m, 1H), 1.98-1.90 (s, 1H), 1.85-1.75 (m, 2H), 1.74-1.60 (m, 7H), 1.59-1.54 (m, 1H).

### Example 28, Compound 43 and 44 Synthesis

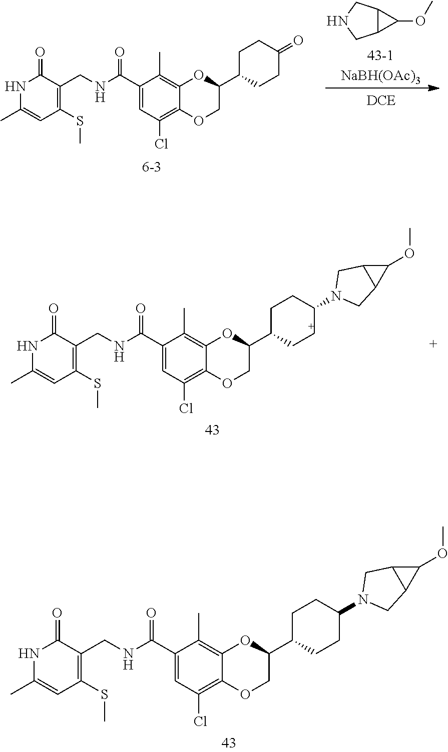

### Step 1, Compound 43 and 44 Synthesis

Following the synthesis method of Example 6, Step 4, except replacing Step 4, dimethylamine with 43-1 (11.0 mg, 0.1 mmol), the same synthesis method was applied, and further resolved by Prep-MPLC to give Compound 43 (4.0 mg, 7.0 mol, 9.0% yield) and 44 (6.0 mg, 0.01 mmol, 13.0% yield), LCMS (ESI⁺) m/z: 588.5 [M+H]⁺.

Compound 43 NMR: ¹H NMR (600 MHz, CDCl₃) δ 6.97 (s, 1H), 6.04 (s, 1H), 4.69-4.51 (m, 1H), 4.49-4.42 (m, 1H), 4.41-4.33 (m, 1H), 4.14-4.10 (m, 1H), 4.08-3.91 (m, 2H), 3.89-3.41 (m, 3H), 3.35 (s, 3H), 2.50 (s, 3H), 2.37 (s, 3H), 2.30-2.15 (m, 4H), 2.15-1.94 (m, 6H), 1.90-1.80 (m, 3H), 1.40-0.97 (m, 3H).

Compound 44 NMR: ¹H NMR (600 MHz, CDCl₃) δ 6.97 (s, 1H), 6.04 (s, 1H), 4.69-4.51 (m, 1H), 4.49-4.35 (m, 1H), 4.35-4.29 (m, 1H), 4.14-4.10 (m, 1H), 4.08-3.91 (m, 2H), 3.90-3.65 (m, 3H), 3.35 (s, 3H), 3.45-3.31 (m, 3H), 2.37 (s, 3H), 2.30-2.15 (m, 4H), 2.15-1.92 (m, 6H), 1.90-1.70 (m, 3H), 1.40-0.97 (m, 3H).

### Example 29, Compound 45 and 46 Synthesis

### Step 1, Compound 45 and 46 Synthesis

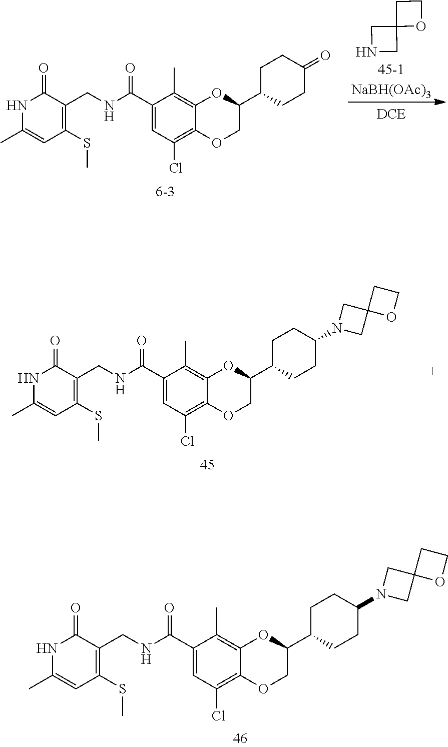

Following the synthesis method of Example 6, Step 4, except replacing Step 4, dimethylamine with 45-1 (10.0 mg, 0.1 mmol), the same synthesis method was applied, and further resolved by Prep-MPLC to give Compound 45 (1.5 mg, 2.6 mol, 3.0% yield) and 46 (1.5 mg, 2.6 mol, 3.0% yield), LCMS (ESI⁺) m/z: 574.4 [M+H]⁺.

Compound 45 NMR: ¹H NMR (600 MHz, DMSO-d₆) δ 11.52 (s, 1H), 8.02 (t, J=4.4 Hz, 1H), 6.90 (s, 1H), 6.08 (s, 1H), 4.49-4.41 (dd, J=11.6, 2.0 Hz, 1H), 4.40-4.33 (t, J=7.6 Hz, 2H), 4.30-4.25 (d, J=4.8 Hz, 2H), 4.10-4.01 (m, 1H), 3.93-3.87 (td, J=7.2, 2.0 Hz, 1H), 3.50-3.44 (m, 2H), 2.97-2.95 (m, 2H), 2.72-2.68 (m, 2H), 2.45 (s, 3H), 2.16 (s, 3H), 2.13 (s, 3H), 1.90-1.78 (m, 1H), 1.77-1.69 (m, 3H), 1.55-1.48 (m, 1H), 1.27-1.05 (m, 3H), 0.97-0.81 (m, 2H).

Compound 46 NMR: ¹H NMR (600 MHz, DMSO-d₆) δ 11.51 (s, 1H), 8.02 (t, J=4.4 Hz, 1H), 6.89 (s, 1H), 6.08 (s, 1H), 4.48-4.34 (m, 3H), 4.32 (d, J=4.4 Hz, 2H), 4.12-3.99 (m, 1H), 3.98-3.89 (m, 1H), 3.46-3.44 (m, 2H), 2.89-2.81 (m, 2H), 2.74 (t, J=7.2 Hz, 2H), 2.45 (m, 3H), 2.16 (s, 3H), 2.13 (s, 3H), 1.64-1.26 (m, 10H).

### Example 30, Compound 47 and 48 Synthesis

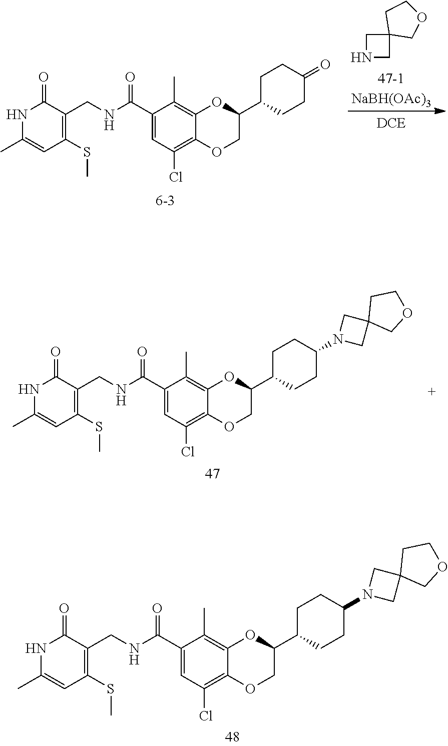

### Step 1, Compound 47 and 48 Synthesis

Following the synthesis method of Example 6, Step 4, except replacing Step 4, dimethylamine with 47-1 (11.0 mg, 0.1 mmol), the same synthesis method was applied, and further resolved by Prep-MPLC to give Compound 47 (1.0 mg, 1.7 mol, 2.0% yield) and 46 (1.0 mg, 1.7 mol, 2.0% yield), LCMS (ESI⁺) m/z: 588.2 [M+H]⁺.

Compound 47 NMR: ¹H NMR (600 MHz, DMSO-d₆) δ 11.52 (s, 1H), 8.02 (t, J=4.4 Hz, 1H), 6.90 (s, 1H), 6.08 (s, 1H), 4.48 (dd, J=11.6, 2.0 Hz, 1H), 4.32 (d, J=4.4 Hz, 2H), 4.15-4.21 (m, 1H), 3.98-3.85 (td, J=7.6, 2.0 Hz, 1H), 3.67-3.59 (m, 4H), 3.09-3.02 (m, 4H), 2.45 (s, 3H), 2.16 (s, 3H), 2.13 (s, 3H), 2.11-1.81 (m, 4H), 1.79-1.69 (m, 3H), 1.58-1.49 (m, 1H), 1.22-1.11 (m, 2H), 0.92-0.81 (m, 2H).

Compound 48 NMR: ¹H NMR (600 MHz, DMSO-d₆) δ 11.52 (s, 1H), 8.02 (t, J=4.4 Hz, 1H), 6.90 (s, 1H), 6.08 (s, 1H), 4.48 (dd, J=11.6, 2.0 Hz, 1H), 4.29 (d, J=4.4 Hz, 2H), 4.08-4.03 (m, 1H), 3.98 (t, J=5.6 Hz, 1H), 3.68-3.62 (m, 4H), 3.07-3.01 (m, 4H), 2.45 (s, 3H), 2.18-2.13 (m, 6H), 2.01 (t, J=7.2 Hz, 2H), 1.58-1.20 (m, 10H).

### Example 31, Compound 49 and 50 Synthesis

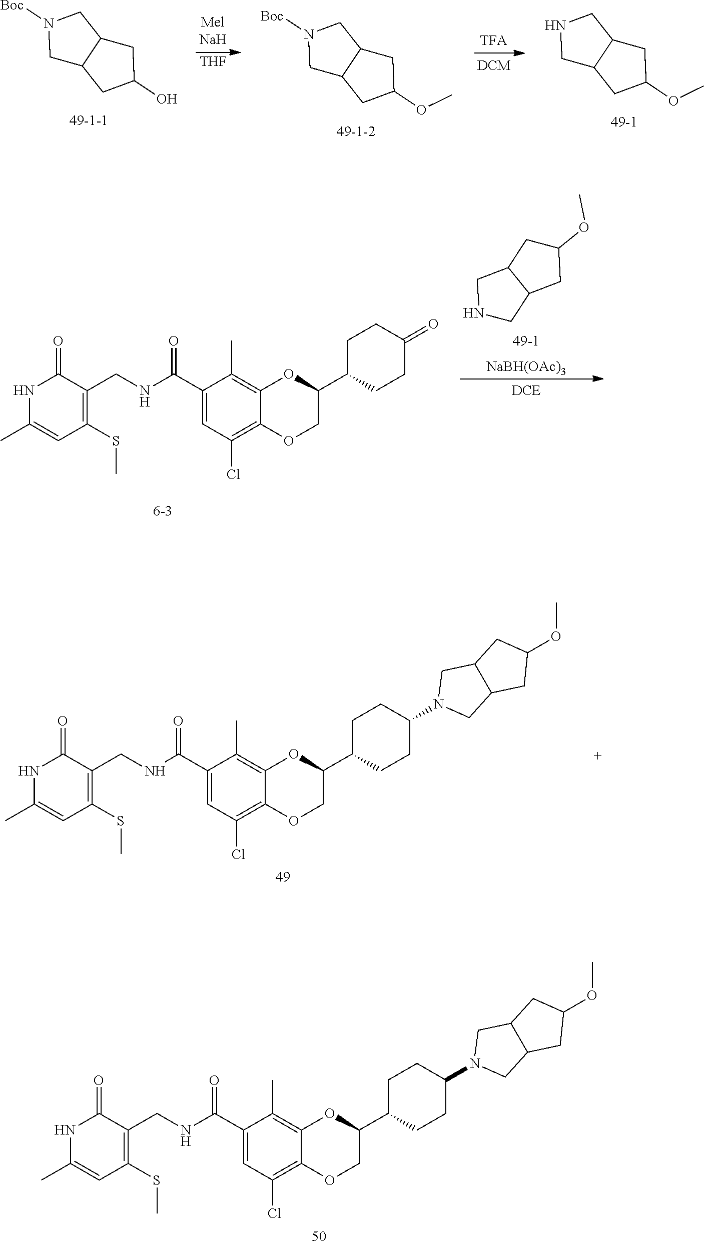

### Step 1, Compound 49-1-2 Synthesis

A single-necked flask was added with substrate 49-1-1 (114.0 mg, 0.5 mmol). The mixture was dissolved with ultra dry THF (2 mL) under stirring, and then at 0° C. added with NaH (15.0 mg, 0.6 mmol) to react for 0.5 hours, added with iodomethane (356.0 mg, 2.5 mmol), 0° C. to react for 1 hour, with LC-MS monitoring. After the completion of the reaction, the mixture was added with water to quench the reaction, and the reaction mixture was extracted with saturated ammonium chloride solution and EA three times. The organic phase was combined, washed with saturated saline solution, and dried with anhydrous sodium sulfate and then condensated under reduced pressure to give the crude product 49-1-2 (155.0 mg, crude), LCMS (ESI⁺) m/z: 241.3 [M+H]⁺.

### Step 2, Compound 49-1 Synthesis

Following the synthesis method of Example 1, Step 4, except replacing Step 4, 1-5 with 49-1-2 (155.0 mg, crude), the same synthesis method was applied, to give Compound 49-1 (70.5 mg, crude), LCMS (ESI⁺) m/z: 141.2 [M+H]⁺.

### Step 3, Compound 49 and 50 Synthesis

Following the synthesis method of Example 6, Step 4, except replacing Step 4, dimethylamine with 49-1 (70.5 mg, crude), the same synthesis method was applied, and further resolved by Prep-MPLC to give Compound 49 (32.0 mg, 0.05 mmol, 26.0% yield) and 50 (48.0 mg, 0.08 mmol, 39.0% yield), LCMS (ESI⁺) m/z: 616.2 [M+H]⁺.

Compound 49 NMR: ¹H NMR (600 MHz, DMSO-d₆) δ 11.52 (s, 1H), 8.07 (t, J=4.2 Hz, 1H), 6.90 (s, 1H), 6.08 (s, 1H), 4.51-4.43 (m, 1H), 4.29 (d, J=4.6 Hz, 2H), 4.09-4.02 (m, 1H), 3.94-3.87 (m, 1H), 3.63-3.61 (m, 1H), 3.18 (s, 3H), 2.45 (s, 4H), 2.42-2.32 (m, 2H), 2.16 (s, 3H), 2.14 (s, 3H), 2.06-1.89 (m, 6H), 1.82-1.70 (m, 2H), 1.60-1.52 (m, 1H), 1.34-1.09 (m, 8H).

Compound 50 NMR: ¹H NMR (600 MHz, DMSO-d₆) δ 11.52 (s, 1H), 8.07 (t, J=4.2 Hz, 1H), 6.89 (s, 1H), 6.08 (s, 1H), 4.47-4.41 (m, 1H), 4.31 (t, J=8.4 Hz, 2H), 4.12-4.05 (m, 1H), 3.99-3.92 (m, 1H), 3.61-3.50 (m, 1H), 3.18 (s, 3H), 2.61-2.58 (m, 2H), 2.44 (s, 3H), 2.40-2.32 (m, 2H), 2.18-2.07 (m, 10H), 1.82-1.78 (m, 2H), 1.73-1.60 (m, 3H), 1.60-1.54 (m 1H), 1.47-1.35 (m, 3H), 1.31-1.20 (m, 3H).

### Example 32, Compound 51 and 52 Synthesis

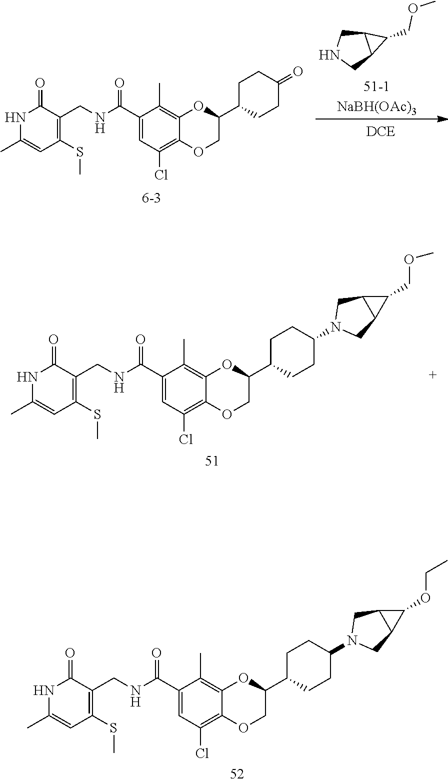

### Step 1, Compound 51 and 52 Synthesis

Following the synthesis method of Example 6, Step 4, except replacing Step 4, dimethylamine with 51-1 (9.0 mg, 0.07 mmol), the same synthesis method was applied, and further resolved by Prep-MPLC to give Compound 51 (4.0 mg, 7.0 mol, 12.0% yield) and 52 (6.0 mg, 0.01 mmol, 16.0% yield), LCMS (ESI⁺) m/z: 602.5 [M+H]⁺.

Compound 51 NMR: ¹H NMR (400 MHz, DMSO-d₆) δ 11.51 (s, 1H), 8.02 (t, J=4.8 Hz, 1H), 6.89 (s, 1H), 6.08 (s, 1H), 4.46-4.44 (m, 1H), 4.27 (d, J=4.4 Hz, 2H), 4.06-4.01 (m, 1H), 3.91-3.87 (m, 1H), 3.19 (s, 3H), 3.13 (d, J=7.2 Hz, 2H), 2.96 (t, J=7.2 Hz, 2H), 2.52.53 (m, 1H), 2.45 (s, 3H), 2.30-2.28 (m, 2H), 2.16 (s, 3H), 2.13 (s, 3H), 2.02-1.96 (m, 2H), 1.92-1.86 (m, 2H), 1.75-1.69 (m, 1H), 1.58-1.52 (m, 1H), 1.26 (s, 2H), 1.21-1.12 (m, 2H), 1.11-1.03 (m. 2H).

Compound 52 NMR: ¹H NMR (400 MHz, DMSO-d₆) δ 11.52 (s, 1H), 8.02 (t, J=4.0 Hz, 1H), 6.89 (s, 1H), 6.08 (s, 1H), 4.43-4.40 (m, 1H), 4.27 (d, J=4.4 Hz, 2H), 4.08-4.04 (m, 1H), 3.96-3.93 (m, 1H), 3.20 (s, 3H), 3.15 (d, J=6.4 Hz, 2H), 2.98 (d, J=8.8 Hz, 2H), 2.55-2.53 (m, 1H), 2.45 (s, 3H), 2.21-2.15 (m, 2H), 2.18 (s, 3H), 2.14 (s, 3H), 1.75-1.70 (m, 2H), 1.69-1.59 (m, 1H), 1.58-1.47 (m, 3H), 1.42-1.31 (m, 3H), 1.30-1.28 (m, 3H).

### Example 33, Compound 53, 54, 55 and 56 Synthesis

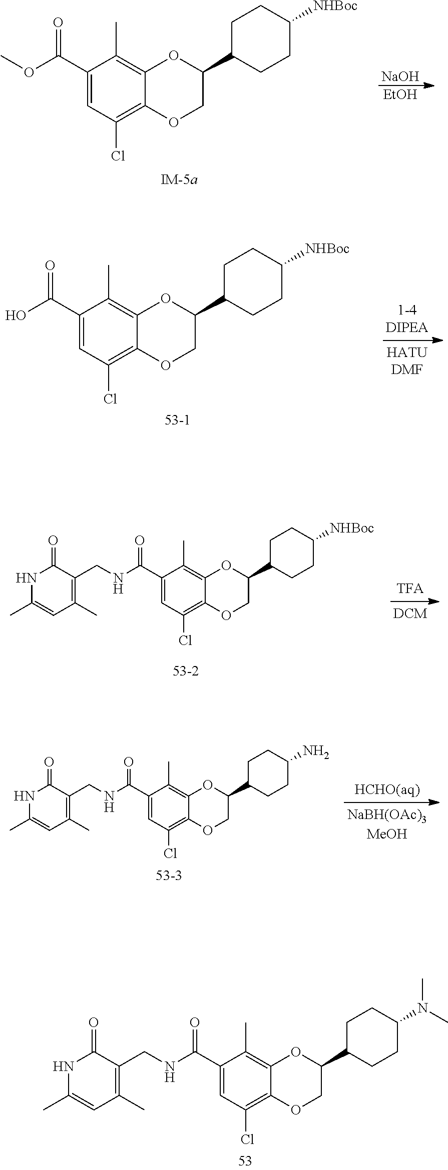

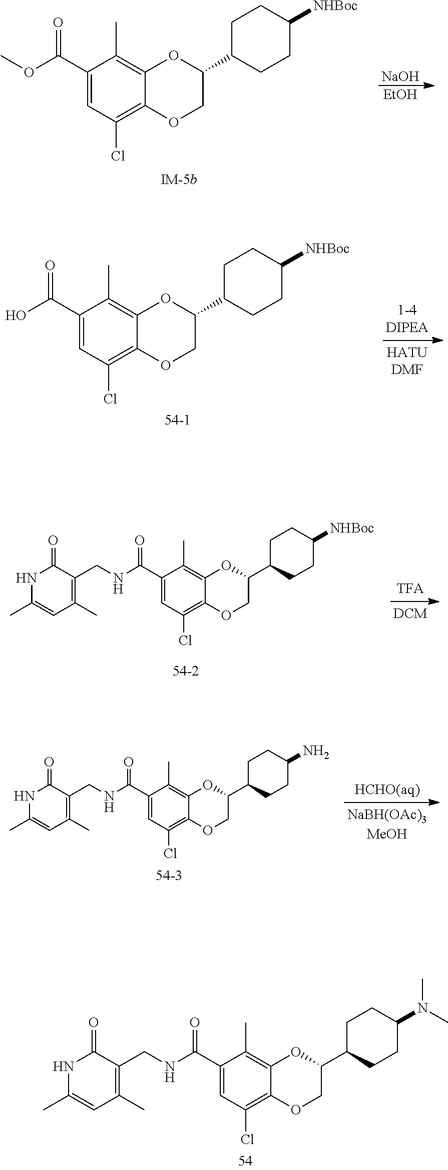

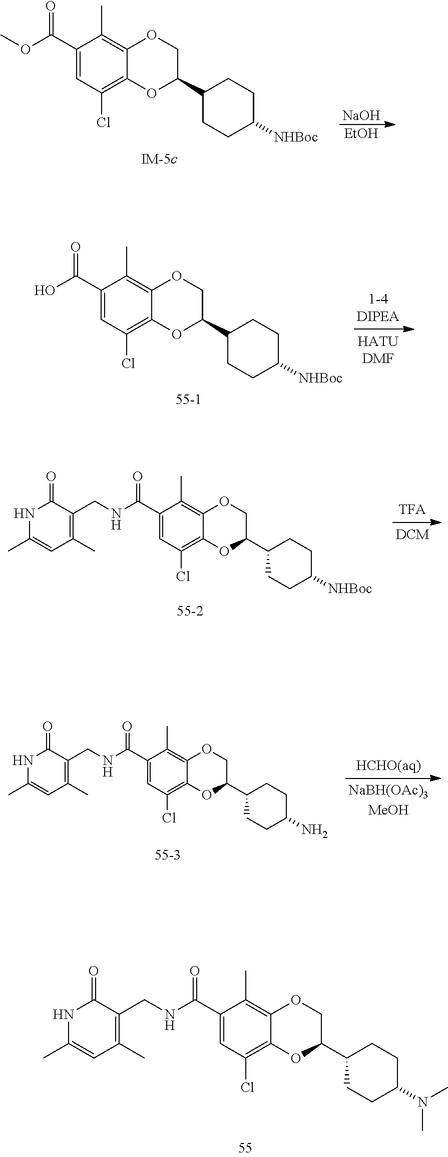

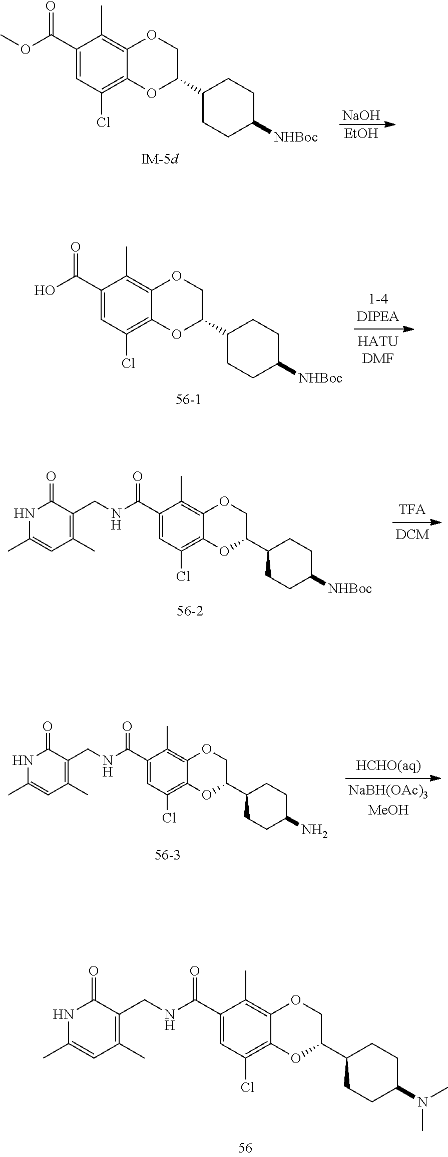

### Step 1, Compound 53-1, 54-1, 55-1 and 56-1 Synthesis

Taking Compound 53-1 as an example:

Following the synthesis method of Example 6, Step 1, except replacing Step 1, IM-3a with IM-5a (50.0 mg, 0.11 mmol), the same synthesis method was applied, to give Compound 53-1 (42.0 mg, crude), LCMS (ESI⁺) m/z: 426.2 [M+H]⁺.

### Step 2, Compound 53-2, 54-2, 55-2 and 56-2 Synthesis

Taking Compound 53-2 as an example:

Following the synthesis method of Example 3, Step 1, except replacing Step 1, 2-3 with 53-1 (42.0 mg, crude), 3-1 with 1-4 (22.8 mg, 0.2 mmol) the same synthesis method was applied, to give Compound 53-2 (30.0 mg, 0.05 mmol, 48.7% yield), LCMS (ESI⁺) m/z: 560.2 [M+H]⁺.

### Step 3, Compound 53-3, 54-3, 55-3 and 56-3 Synthesis

Taking Compound 53-3 as an example:

Following the synthesis method of Example 3, Step 2, except replacing Step 2, 3-2 with 53-2 (30.0 mg, 0.05 mmol), the same synthesis method was applied, to give Compound 53-3 (18.0 mg, crude), LCMS (ESI⁺) m/z: 460.2 [M+H]⁺.

### Step 4, Compound 53, 54, 55 and 56 Synthesis

Taking Compound 53 as an example:

Following the synthesis method of Example 2, Step 5, except replacing Step 5, 2-6 with 53-3 (18.0 mg, crude), the same synthesis method was applied, to give Compound 53 (5.0 mg, 0.01 mmol, 20.5% yield), LCMS (ESI⁺) m/z: 488.2 [M+H]⁺.

Compound 53 NMR: ¹H NMR (600 MHz, DMSO-d₆) δ 11.46 (s, 1H), 8.11 (t, J=4.8 Hz, 1H), 6.88 (s, 1H), 5.85 (s, 1H), 4.46 (dd, J=11.4, 2.4 Hz, 1H), 4.22 (d, J=4.8 Hz, 2H), 4.07-4.05 (m, 1H), 3.93-3.89 (m, 1H), 2.17 (s, 3H), 2.16 (s, 6H), 2.13-2.10 (m, 7H), 2.05-2.01 (m, 1H), 1.88-1.82 (m, 2H), 1.80-1.78 (m, 1H), 1.57-1.51 (m, 1H), 1.20-1.13 (m, 4H).

Compound 55 NMR: ¹H NMR (600 MHz, DMSO-d₆) δ 11.46 (s, 1H), 8.11 (t, J=4.2 Hz, 1H), 6.90 (s, 1H), 5.86 (s, 1H), 4.46-4.43 (m, 1H), 4.22 (d, J=4.8 Hz, 2H), 4.07-4.05 (m, 1H), 3.93-3.89 (m, 1H), 2.17 (s, 3H), 2.16 (s, 6H), 2.11 (s, 6H), 2.05-1.97 (m, 2H), 1.85-1.81 (m, 2H), 1.80-1.76 (m, 1H), 1.57-1.51 (m, 1H), 1.18-1.12 (m, 4H).

Compound 56 NMR: ¹H NMR (600 MHz, DMSO-d₆) δ 11.46 (s, 1H), 8.11 (t, J=4.2 Hz, 1H), 6.90 (s, 1H), 5.86 (s, 1H), 4.46-4.42 (m, 1H), 4.24-4.20 (m, 2H), 4.07-4.02 (m, 1H), 3.97-3.94 (m, 1H), 2.17 (m, 9H), 2.13-2.09 (m, 6H), 2.09-2.07 (m, 1H), 2.07-2.04 (m, 1H), 1.88-1.82 (m, 2H), 1.82-1.76 (m, 1H), 1.57-1.51 (m, 1H), 1.20-1.12 (m, 4H).

### Example 34, Compound 57 and 58 Synthesis

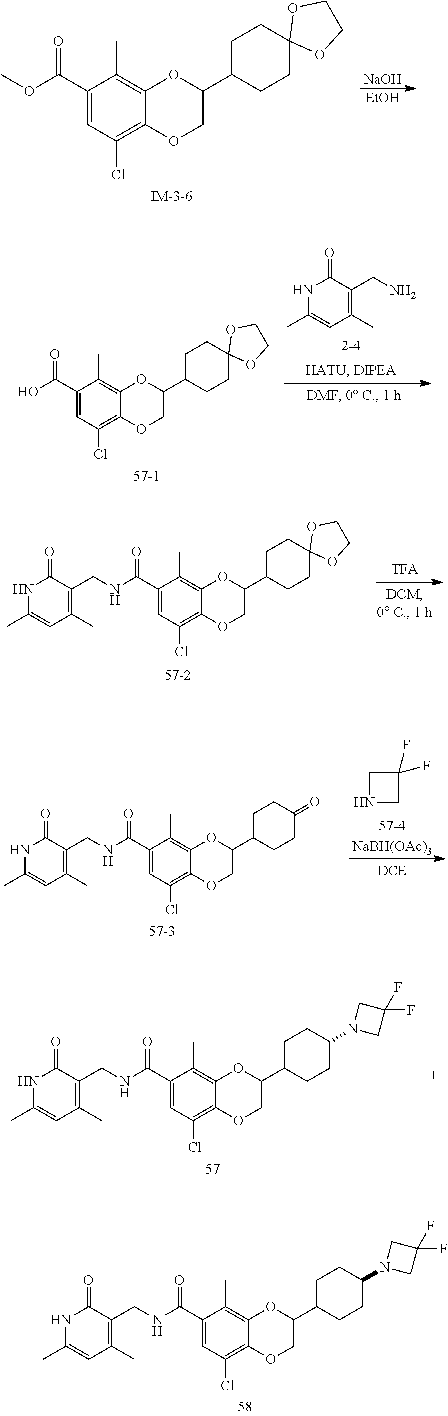

### Step 1, Compound 57-1 Synthesis

A single-necked flask was added with substrate IM-3-6 (500.0 mg, 1.3 mmol). The mixture was dissolved with EtOH (3 mL) to dissolve, and then added with 2 N NaOH (3 mL), 80° C. to react for 1 hour, with LC-MS monitoring. After the completion of the reaction, the mixture was added with 1 N HCl to adjust to pH=6-7, and then added with EA solution to extract three times. The organic phase was combined, washed with saturated saline solution, and dried with anhydrous sodium sulfate and then condensated under reduced pressure, to give the crude product 57-1 (502.0 mg, crude). LCMS (ESI⁺) m/z: 368.8 [M+H]⁺

### Step 2, Compound 57-2 Synthesis

Following the synthesis method of Example 1, Step 3, except replacing Step 3, 1-3 with 57-1 (502.0 mg, crude), the same synthesis method was applied, to give Compound 57-2 (705.0 mg, crude), LCMS (ESI⁺) m/z: 503.1 [M+H]⁺.

### Step 3, Compound 57-3 Synthesis

Following the synthesis method of Example 3, Step 2, except replacing Step 2, 3-2 with 57-2 (705.0 mg, crude), the same synthesis method was applied, to give Compound 57-3 (620.0 mg, crude), LCMS (ESI⁺) m/z: 458.9 [M+H]⁺.

### Step 4, Compound 57 and 58 Synthesis

Following the synthesis method of Example 6, Step 4, except replacing Step 4, 6-3 with 57-3 (80.0 mg, 0.17 mmol), dimethylamine with 57-4 (49.0 mg, 0.5 mmol), the same synthesis method was applied. The obtained compounds was subjected to medium pressure liquid chromatography to give Compound 57 (20.0 mg, 0.04 mmol, 22.0% yield) and 58 (3.0 mg, 5.6 mol, 3.3% yield), LCMS (ESI⁺) m/z: 536.2 [M+H]⁺.

Compound 57 NMR: ¹H NMR (600 MHz, CDCl₃) δ 10.61 (s, 1H), 7.13 (s, 1H), 6.92 (s, 1H), 5.95 (s, 1H), 4.52-4.43 (m, 2H), 4.37 (d, J=4.2 Hz, 1H), 4.02-3.99 (m, 1H), 3.82-3.80 (m, 1H), 3.57 (t, J=11.8 Hz, 4H), 2.37 (s, 3H), 2.23-2.20 (m, 6H), 2.12-2.07 (m, 2H), 1.85-1.81 (m, 2H), 1.79-1.75 (m, 1H), 1.29-0.98 (m, 5H).

Compound 58 NMR: ¹H NMR (600 MHz, CDCl₃) δ 10.68 (s, 1H), 7.03-7.01 (m, 1H), 6.95 (s, 1H), 5.95 (s, 1H), 4.49-4.46 (m, 2H), 4.38-4.34 (m, 1H), 4.13-4.00 (m, 3H), 3.94-3.68 (m, 3H), 2.90-2.60 (m, 1H), 2.38 (s, 3H), 2.24 (s, 3H), 2.21 (s, 3H), 2.00-1.92 (m, 1H), 1.80-1.69 (m, 8H).

### Example 35, Compound 59 and 60 Synthesis

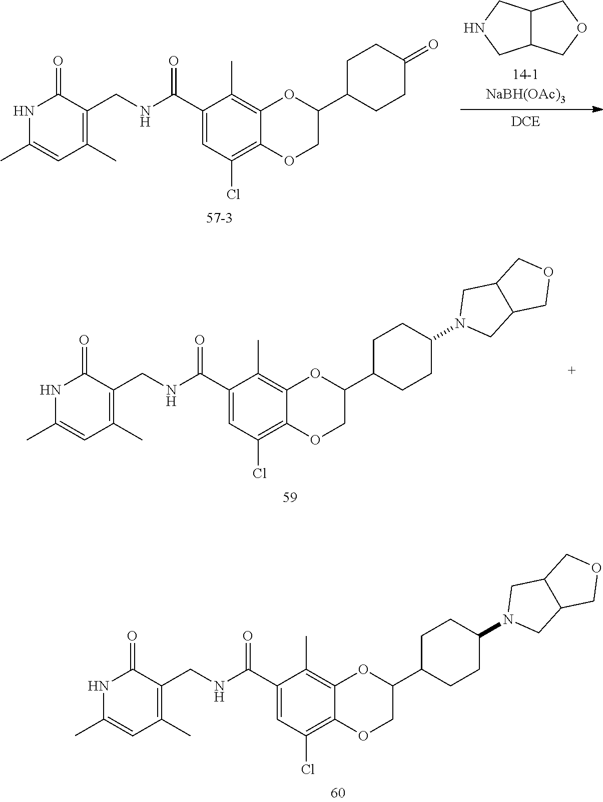

### Step 1, Compound 59 and 60 Synthesis

Following the synthesis method of Example 6, Step 4, except replacing Step 4, 6-3 with 57-3 (50.0 mg, 0.1 mmol), dimethylamine with 14-1 (37.0 mg, 0.3 mmol), the same synthesis method was applied. The obtained compounds was subjected to medium pressure liquid chromatography to give Compound 59 (10.0 mg, 0.02 mmol, 16.5% yield) and 60 (10.0 mg, 0.02 mmol, 16.5% yield), LCMS (ESI⁺) m/z: 556.2 [M+H]⁺.

Compound 59 NMR: ¹H NMR (600 MHz, CDCl₃) δ 10.46 (s, 1H), 7.11 (t, J=5.4 Hz, 1H), 6.96 (s, 1H), 5.93 (s, 1H), 4.49 (d, J=6.0 Hz, 2H), 4.37 (dd, J=10.8, 1.8 Hz, 1H), 4.01-3.99 (m, 1H), 3.86-3.82 (m, 1H), 3.74-3.72 (m, 2H), 3.65-3.63 (m, 2H), 3.19-3.01 (m, 2H), 2.45-2.39 (m, 2H), 2.37 (s, 3H), 2.24 (s, 3H), 2.22 (s, 3H), 2.16-2.11 (m, 3H), 1.88-1.84 (m, 1H), 1.74-1.66 (m, 5H), 1.34-1.14 (m, 3H).

Compound 60 NMR: ¹H NMR (600 MHz, CDCl₃) δ 10.12 (s, 1H), 7.07 (dd, J=11.2, 5.4 Hz, 1H), 6.95 (s, 1H), 5.92 (s, 1H), 4.49 (t, J=5.4 Hz, 2H), 4.34 (d, J=10.8 Hz, 2H), 4.19-4.16 (m, 1H), 4.09-3.95 (m, 2H), 3.86-3.80 (m, 2H), 3.75-3.70 (m, 1H), 3.65-3.55 (m, 2H), 3.48-3.34 (m, 2H), 3.09-3.01 (m, 1H), 2.52-2.48 (m, 2H), 2.50 (s, 3H), 2.37 (s, 3H), 2.24 (s, 3H), 2.10-2.05 (m, 2H), 1.89-1.77 (m, 2H), 1.75-1.68 (m, 2H), 1.63-1.59 (m, 2H).

### Example 36, Compound 61 and 62 Synthesis

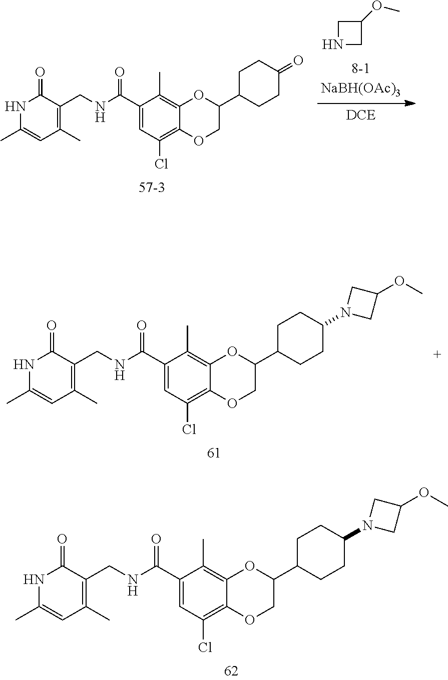

### Step 1, Compound 61 and 62 Synthesis

Following the synthesis method of Example 6, Step 4, except replacing Step 4, 6-3 with 57-3 (20.0 mg, 0.04 mmol), dimethylamine with 8-1 (7.6 mg, 0.09 mmol), the same synthesis method was applied. The obtained compounds was subjected to medium pressure liquid chromatography to give Compound 61 (4.4 mg, 8.3 mol, 19.0% yield) and 62 (4.4 mg, 8.3 mol, 19.0% yield), LCMS (ESI⁺) m/z: 530.6 [M+H]⁺.

Compound 61 NMR: ¹H NMR (600 MHz, CDCl₃) δ 10.81 (s, 1H), 7.13 (t, J=6.0 Hz, 1H), 6.97 (s, 1H), 5.94 (s, 1H), 4.55 (d, J=5.4 Hz, 2H), 4.40 (dd, J=9.0, 2.4 Hz, 1H), 4.30-4.26 (m, 1H), 4.25-4.10 (m, 2H), 4.07-3.96 (m, 1H), 3.86-3.82 (m, 1H), 3.46-3.42 (m, 2H), 3.30 (s, 3H), 2.70-2.65 (m, 1H), 2.37 (s, 3H), 2.24 (s, 3H), 2.22 (s, 3H), 2.17-2.12 (m, 1H), 2.05-1.95 (m, 3H), 1.90-1.84 (m, 2H), 1.75-1.69 (m, 3H).

Compound 62 NMR: ¹H NMR (600 MHz, CDCl₃) δ 12.04 (s, 1H), 7.03 (t, J=6.0 Hz, 1H), 6.94 (s, 1H), 5.94 (s, 1H), 4.65-4.56 (m, 2H), 4.52-4.44 (m, 3H), 4.37-4.29 (m, 1H), 4.28-4.24 (m, 1H), 4.22-4.16 (m, 1H), 3.74-3.72 (m, 1H), 3.60-3.55 (m, 2H), 3.32 (s, 3H), 3.24-3.19 (m, 1H), 2.38 (s, 3H), 2.24 (s, 3H), 2.19 (s, 3H), 2.08-2.05 (m, 1H), 1.95-1.86 (m, 2H), 1.82-1.76 (m, 2H), 1.74-1.60 (m, 3H).

### Example 37, Compound 63 Synthesis

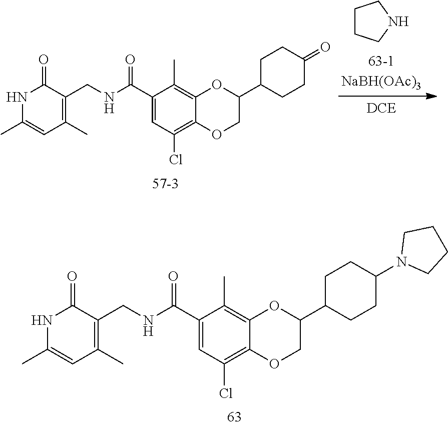

### Step 1, Compound 63 Synthesis

Following the synthesis method of Example 6, Step 4, except replacing Step 4, 6-3 with 57-3 (46.0 mg, 0.1 mmol), dimethylamine with 63-1 (11.0 mg, 0.2 mmol), the same synthesis method was applied to give Compound 63 (40.0 mg, 0.08 mmol, 77.8% yield), LCMS (ESI⁺) m/z: 514.2 [M+H]⁺.

Compound 63 NMR: ¹H NMR (600 MHz, DMSO-d₆) δ 11.46 (s, 1H), 8.12 (t, J=4.8 Hz, 1H), 6.88 (s, 1H), 5.53 (s, 1H), 4.48-4.42 (m, 1H), 4.26 (d, J=5.4 Hz, 2H), 4.11-3.92 (m, 2H), 2.17 (s, 3H), 2.11 (m, 6H), 2.07-1.97 (m, 2H), 1.85-1.43 (m, 12H), 1.28-1.12 (m, 4H).

### Example 38, Compound 64 and 65 Synthesis

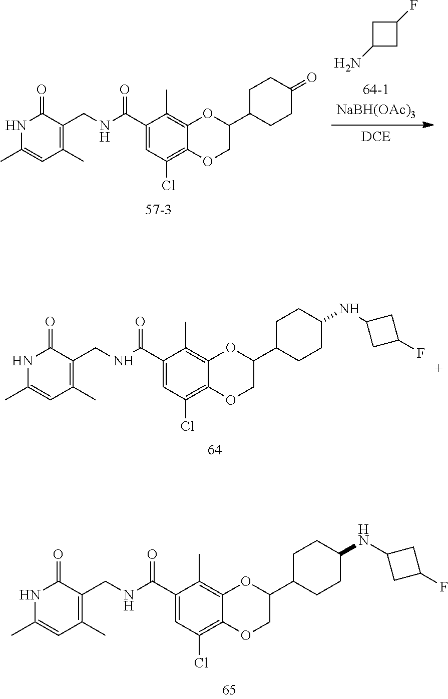

### Step 1, Compound 64 and 65 Synthesis

Following the synthesis method of Example 6, Step 4, except replacing Step 4, 6-3 with 57-3 (46.0 mg, 0.1 mmol), dimethylamine with 64-1 (13.0 mg, 0.2 mmol), the same synthesis method was applied. The obtained compounds was subjected to medium pressure liquid chromatography to give Compound 64 (15.0 mg, 0.03 mmol, 28.2% yield) and 65 (20.0 mg, 0.04 mmol, 37.6% yield), LCMS (ESI⁺) m/z: 532.3 [M+H]⁺.

Compound 64 NMR: ¹H NMR (600 MHz, DMSO-d₆) δ 11.46 (s, 1H), 8.11 (t, J=4.8 Hz, 1H), 6.88 (s, 1H), 5.86 (s, 1H), 5.35-5.08 (m, 1H), 4.48 (dd, J=12.0, 2.4 Hz, 1H), 4.22 (d, J=5.4 Hz, 2H), 4.06-4.02 (m, 1H), 3.91-3.88 (m, 1H), 3.56-3.51 (m, 1H), 2.32-2.23 (m, 3H), 2.17 (s, 3H), 2.11 (m, 6H), 2.07-2.01 (m, 2H), 1.98-1.92 (m, 1H), 1.89-1.82 (m, 2H), 1.73-1.69 (m, 1H), 1.56-1.51 (m, 1H), 1.28-1.13 (m, 3H), 1.01-0.92 (m, 2H).

Compound 65 NMR: ¹H NMR (600 MHz, DMSO-d₆) δ 11.46 (s, 1H), 8.11 (t, J=4.8 Hz, 1H), 6.88 (s, 1H), 5.86 (s, 1H), 5.25-5.10 (m, 1H), 4.48 (dd, J=11.4, 1.8 Hz, 1H), 4.22 (d, J=4.8 Hz, 2H), 4.09-4.03 (m, 1H), 4.02-3.98 (m, 1H), 3.52-3.44 (m, 1H), 2.21-2.13 (m, 1H), 2.35-2.26 (m, 2H), 2.17 (s, 3H), 2.11 (m, 8H), 1.68-1.49 (m, 10H).

### Example 39, Compound 66 and 67 Synthesis

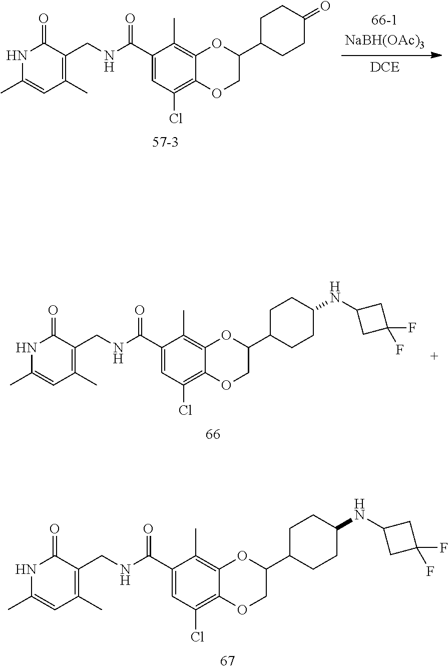

### Step 1, Compound 66 and 67 Synthesis

Following the synthesis method of Example 6, Step 4, except replacing Step 4, 6-3 with 57-3 (46.0 mg, 0.1 mmol), dimethylamine with 66-1 (16.0 mg, 0.15 mmol), the same synthesis method was applied. The obtained compounds was subjected to medium pressure liquid chromatography to give Compound 66 (12.0 mg, 0.02 mmol, 21.8% yield) and 67 (18.0 mg, 0.03 mmol, 32.7% yield), LCMS (ESI⁺) m/z: 550.3 [M+H]⁺.

Compound 66 NMR: ¹H NMR (600 MHz, DMSO-d₆) δ 11.46 (s, 1H), 8.11 (t, J=4.8 Hz, 1H), 6.88 (s, 1H), 5.86 (s, 1H), 4.48 (dd, J=11.4, 1.8 Hz, 1H), 4.22 (d, J=5.4 Hz, 2H), 4.08-4.04 (m, 1H), 3.92-3.89 (m, 1H), 3.22-3.17 (m, 1H), 2.77-2.68 (m, 2H), 2.35-2.21 (m, 3H), 2.17 (s, 3H), 2.13-2.10 (m, 6H), 1.95-1.93 (m, 1H), 1.88-1.84 (m, 2H), 1.74-1.70 (m, 1H), 1.58-1.49 (m, 1H), 1.25-1.11 (m, 3H), 1.11-0.93 (m, 2H).

Compound 67 NMR: ¹H NMR (600 MHz, DMSO-d₆) δ 11.46 (s, 1H), 8.11 (t, J=4.8 Hz, 1H), 6.88 (s, 1H), 5.86 (s, 1H), 4.48 (dd, J=12.0, 2.4 Hz, 1H), 4.22 (d, J=4.8 Hz, 2H), 4.08-4.05 (m, 1H), 4.01-3.97 (m, 1H), 3.18-3.11 (m, 1H), 2.78-2.66 (m, 3H), 2.36-2.24 (m, 2H), 2.17 (s, 3H), 2.11 (m, 6H), 1.68-1.48 (m, 10H).

### Example 40, Compound 68 and 69 Synthesis

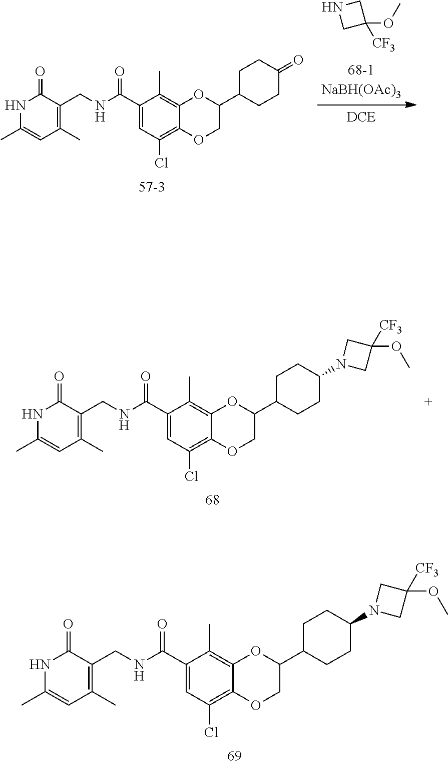

### Step 1, Compound 68 and 69 Synthesis

Following the synthesis method of Example 6, Step 4, except replacing Step 4, 6-3 with 57-3 (50.0 mg, 0.1 mmol), dimethylamine with 68-1 (85.0 mg, 0.55 mmol), the same synthesis method was applied. The obtained compounds was subjected to medium pressure liquid chromatography to give Compound 68 (9.0 mg, 0.015 mmol, 13.8% yield) and 69 (10.0 mg, 0.017 mmol, 15.3% yield), LCMS (ESI⁺) m/z: 598.6 [M+H]⁺.

### Example 41, Compound 70 and 71 Synthesis

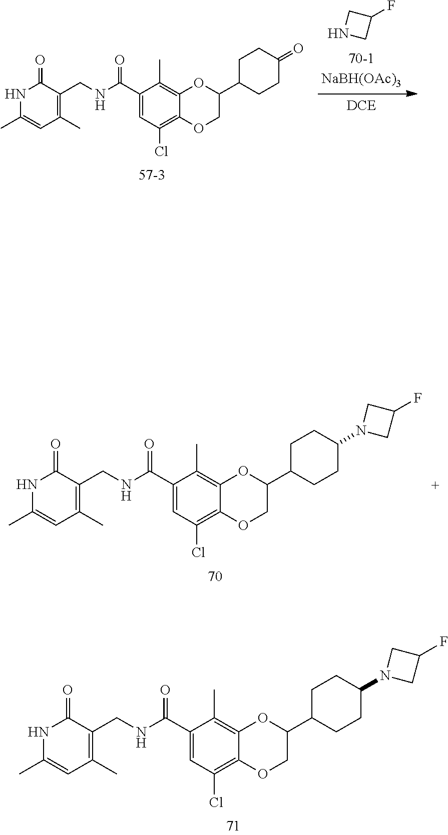

### Step 1, Compound 70 and 71 Synthesis

Following the synthesis method of Example 6, Step 4, except replacing Step 4, 6-3 with 57-3 (46.0 mg, 0.1 mmol), dimethylamine with 70-1 (9.0 mg, 0.1 mmol), the same synthesis method was applied. The obtained compound was subjected to Prep-MPLC to give Compound 70 (5.0 mg, 0.01 mmol, 9.7% yield) and 71 (12.0 mg, 0.02 mmol, 23.2% yield), LCMS (ESI⁺) m/z: 518.3 [M+H]⁺.

Compound 70 NMR: ¹H NMR (400 MHz, DMSO-d₆) δ 11.47 (s, 1H), 8.12-8.10 (m, 1H), 6.89-6.87 (m, 1H), 5.85 (s, 1H), 5.19-5.04 (m, 1H), 4.50-4.45 (m, 1H), 4.30-4.20 (m, 2H), 4.10-4.02 (m, 1H), 3.98-3.90 (m, 1H), 3.52-3.50 (m, 2H), 3.02-2.98 (m, 2H), 2.17 (s, 3H), 2.13-2.11 (m, 6H), 2.02-1.94 (m, 1H), 1.78-1.72 (m, 2H), 1.60-1.51 (m, 1H), 1.29-1.15 (m, 4H), 0.92-0.85 (m, 2H).

Compound 71 NMR: ¹H NMR (400 MHz, DMSO-d₆) δ 11.49 (s, 1H), 8.12-8.10 (m, 1H), 6.89-6.87 (m, 1H), 5.85 (s, 1H), 5.20-5.04 (m, 1H), 4.42-4.38 (m, 1H), 4.25-4.18 (m, 2H), 4.10-4.02 (m, 1H), 3.98-3.88 (m, 1H), 3.54-3.46 (m, 2H), 2.99-2.90 (m, 2H), 2.17 (s, 3H), 2.11-2.08 (m, 6H), 1.65-1.49 (m, 6H), 1.42-1.32 (m, 4H).

### Example 42, Compound 72, 73, 74 and 75 Synthesis

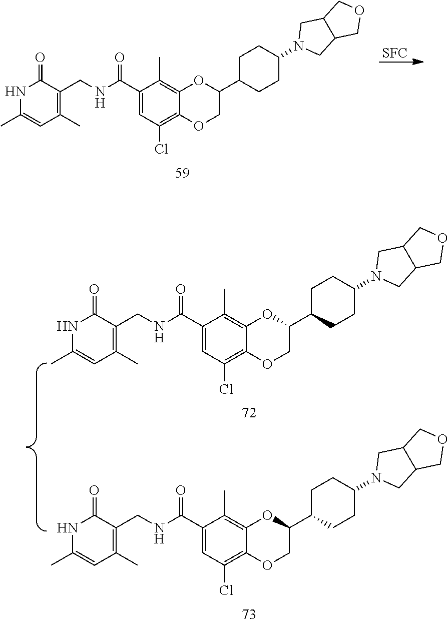

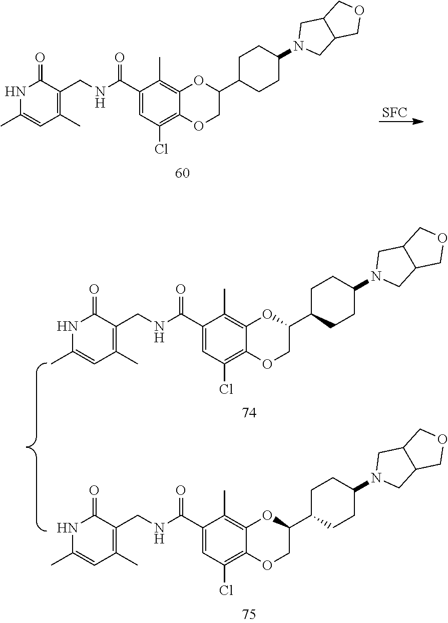

### Step 1, Compound 72 and 73 Synthesis

Compound 72 and 73 could be obtained by SFC resolution of Compound 59, LCMS (ESI⁺) m/z: 556.3 [M+H]⁺

Compound 72 NMR: ¹H NMR (400 MHz, CDCl₃) δ 12.05 (s, 1H), 7.23-7.21 (m, 1H), 6.96 (s, 1H), 5.95 (s, 1H), 4.50 (d, J=5.4 Hz, 2H), 4.38 (d, J=10.8 Hz, 1H), 4.04-3.98 (m, 1H), 3.84-3.78 (m, 1H), 3.75-3.68 (m, 2H), 3.66-3.62 (m, 2H), 3.12-2.98 (m, 2H), 2.36 (s, 3H), 2.29-2.27 (m, 3H), 2.24-2.22 (m, 6H), 2.12-2.02 (m, 4H), 1.83-1.75 (m, 2H), 1.65-1.58 (m, 2H), 1.31-1.19 (m, 3H).

Compound 73 NMR: ¹H NMR (400 MHz, CDCl₃) δ 12.00 (s, 1H), 7.20 (t, J=5.4 Hz, 1H), 6.96 (s, 1H), 5.95 (s, 1H), 4.55-4.46 (m, 2H), 4.39-4.24 (d, J=9.6 Hz, 1H), 4.04-3.92 (m, 4H), 3.58-3.42 (m, 2H), 2.90-2.60 (m, 2H), 2.51-2.45 (m, 3H), 2.37 (s, 3H), 2.24-2.21 (m, 6H), 1.95-1.60 (m, 10H), 1.51-1.48 (m, 1H).

### Step 2, Compound 74 and 75 Synthesis

Compound 74 and 75 could be obtained by SFC resolution of Compound 60. LCMS (ESI⁺) m/z: 556.1 [M+H]⁺.

Compound 74 NMR: ¹H NMR (400 MHz, CDCl₃) δ 11.97 (s, 1H), 7.20 (t, J=6.0 Hz, 1H), 6.95 (s, 1H), 5.95 (s, 1H), 4.55-4.46 (m, 2H), 4.42-4.35 (d, J=9.6 Hz, 1H), 4.10-3.92 (m, 2H), 3.94-3.78 (m, 2H), 3.58-3.47 (m, 2H), 2.51-2.42 (m, 3H), 2.37 (s, 3H), 2.26-2.22 (m, 7H), 1.90-1.63 (m, 9H), 1.50-1.47 (m, 3H).

Compound 75 NMR: ¹H NMR (400 MHz, CDCl₃) δ 11.98 (s, 1H), 7.19 (t, J=5.4 Hz, 1H), 6.96 (s, 1H), 5.96 (s, 1H), 4.55 (d, J=5.4 Hz 2H), 4.42 (d, J=10.2 Hz, 1H), 4.07-3.94 (i, 1H), 3.85-3.77 (i, 1H), 3.76-3.51 (i, 4H), 3.18-3.02 (i, 2H), 2.37 (s, 3H), 2.35-2.18 (m, 8H), 2.15-1.98 (m, 5H), 1.82-1.78 (m, 1H), 1.68-1.59 (m, 1H), 1.42-1.22 (m, 5H).

### Example 43, Compound 76 and 77 Synthesis

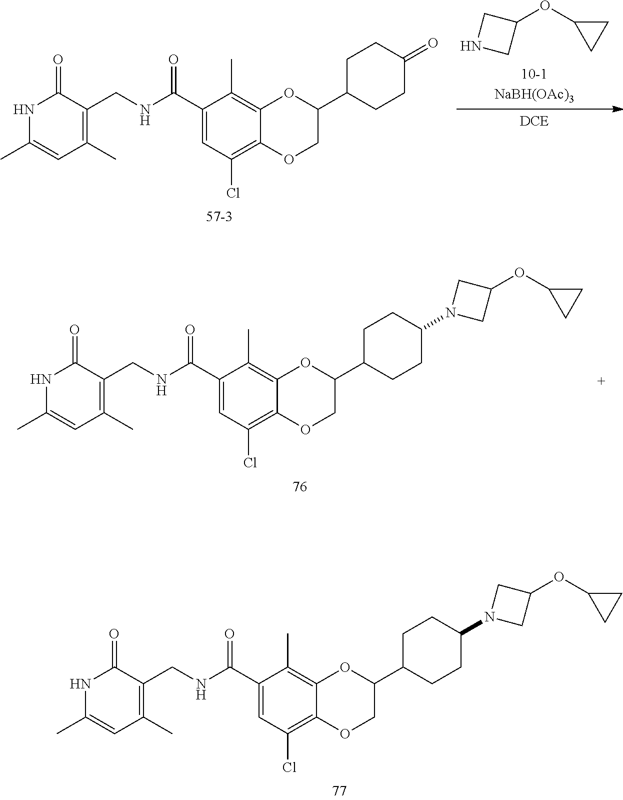

### Step 1, Compound 76 and 77 Synthesis

Following the synthesis method of Example 6, Step 4, except replacing Step 4, 6-3 with 57-3 (40.0 mg, 0.09 mmol), dimethylamine with 10-1 (12.0 mg, 0.1 mmol), the same synthesis method was applied. The obtained compound was subjected to Prep-MPLC to give Compound 76 (7.5 mg, 13.5 mol, 15.5% yield) and 77 (7.5 mg, 13.5 mol, 15.5% yield), LCMS (ESI⁺) m/z: 556.3 [M+H]⁺.

Compound 76 NMR: ¹H NMR (600 MHz, DMSO-d₆) δ 11.46 (s, 1H), 8.11 (t, J=4.8 Hz, 1H), 6.87 (s, 1H), 5.85 (s, 1H), 4.48-4.43 (m, 1H), 4.22 (d, J=4.8 Hz, 2H), 4.09-4.02 (m, 2H), 3.94-3.88 (m, 1H), 3.49-3.45 (m, 2H), 3.23-3.19 (m, 1H), 2.78-2.72 (m, 2H), 2.16 (s, 3H), 2.13-2.10 (m, 6H), 1.96-1.82 (m, 2H), 1.79-1.69 (m, 3H), 1.56-1.50 (m, 1H), 1.56-1.51 (m, 2H), 0.92-0.80 (m, 2H), 0.46-0.43 (m, 2H), 0.42-0.38 (m, 2H).

Compound 77 NMR: ¹H NMR (600 MHz, DMSO-d₆) δ 11.46 (s, 1H), 8.11 (t, J=4.8 Hz, 1H), 6.87 (s, 1H), 5.85 (s, 1H), 4.42 (dd, J=11.4, 1.8 Hz, 1H), 4.22 (d, J=5.4 Hz, 2H), 4.12-4.02 (m, 2H), 3.94-3.92 (m, 1H), 3.50-3.44 (m, 2H), 3.24-3.19 (m, 1H), 2.72 (t, J=6.0 Hz, 2H), 2.24-2.19 (m, 1H), 2.18-2.16 (m, 3H), 2.12-2.09 (m, 6H), 1.62-1.29 (m, 9H), 0.48-0.43 (m, 2H), 0.43-0.38 (m, 2H).

### Example 44, Compound 78 and 79 Synthesis

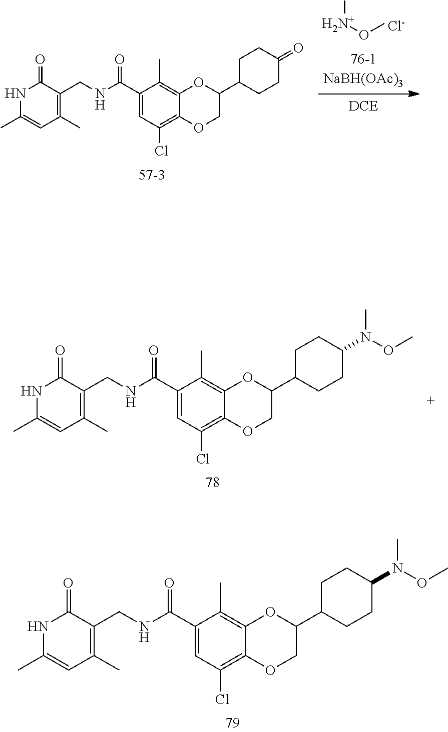

### Step 1, Compound 78 and 79 Synthesis

Following the synthesis method of Example 6, Step 4, except replacing Step 4, 6-3 with 57-3 (100.0 mg, 0.22 mmol), dimethylamine with 78-1 (40.0 mg, 0.4 mmol), the same synthesis method was applied. The obtained compound was subjected to Prep-MPLC to give Compound 78 (24.0 mg, 0.05 mmol, 21.8% yield) and 79 (18.0 mg, 0.04 mmol, 16.4% yield), LCMS (ESI⁺) m/z: 504.2 [M+H]⁺.

Compound 78 NMR: ¹H NMR (600 MHz, DMSO-d₆) δ 11.46 (s, 1H), 8.11 (t, J=4.8 Hz, 1H), 6.88 (s, 1H), 5.85 (s, 1H), 4.48 (dd, J=10.8 Hz, 1.8 Hz, 1H), 4.22 (d, J=5.4 Hz, 2H), 4.12-4.06 (m, 1H), 4.05-4.01 (m, 1H), 3.42 (s, 3H), 2.48 (s, 3H), 2.47-2.45 (m, 1H), 2.17 (s, 3H), 2.11 (s, 3H), 2.10 (s, 3H), 1.85-1.52 (m, 6H), 1.52-1.43 (m, 3H).

Compound 79 NMR: ¹H NMR (600 MHz, DMSO-d₆) δ 11.46 (s, 1H), 8.11 (t, J=5.4 Hz, 1H), 6.88 (s, 1H), 5.85 (s, 1H), 4.48 (dd, J=12.0, 2.4 Hz, 1H), 4.22 (d, J=4.8 Hz, 2H), 4.08-4.02 (m, 1H), 3.93-3.88 (m, 1H), 3.37 (s, 3H), 2.48 (s, 3H), 2.47-2.43 (m, 1H), 2.17 (s, 3H), 2.17 (s, 3H), 2.11 (s, 3H), 2.02-1.91 (m, 3H), 1.81-1.76 (m, 1H), 1.60-1.53 (m, 1H), 1.24-1.10 (m, 4H).

### Example 45, Compound 80 and 81 Synthesis

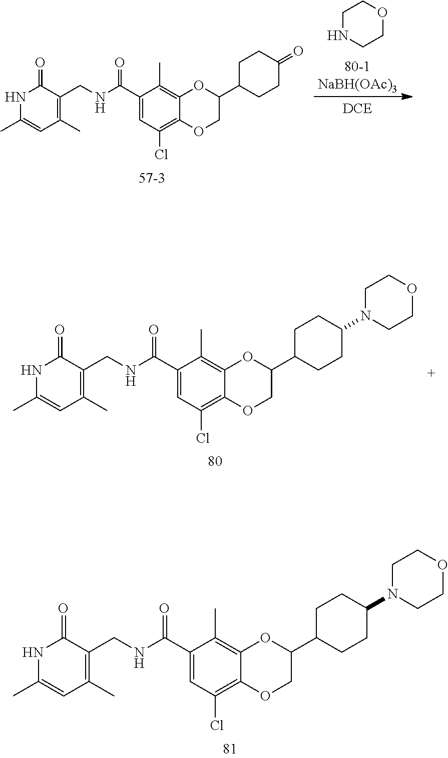

### Step 1, Compound 80 and 81 Synthesis

Following the synthesis method of Example 6, Step 4, except replacing Step 4, 6-3 with 57-3 (50.0 mg, 0.1 mmol), dimethylamine with 80-1 (19.0 mg, 0.2 mmol), the same synthesis method was applied. The obtained compound was subjected to Prep-MPLC to give Compound 80 (10.0 mg, 18.9 mol, 17.3% yield) and 81 (5.0 mg, 9.4 mol, 8.7% yield), LCMS (ESI⁺) m/z: 530.6 [M+H]⁺.

Compound 80 NMR: ¹H NMR (600 MHz, CDCl₃) δ 10.81 (s, 1H), 7.13 (t, J=6.0 Hz, 1H), 6.97 (s, 1H), 5.94 (s, 1H), 4.51 (d, J=5.4 Hz, 2H), 4.38 (d, J=11.4, 2.4 Hz, 1H), 4.30-4.26 (m, 1H), 4.25-4.19 (m, 2H), 4.03-3.98 (m, 1H), 3.86-3.82 (m, 1H), 3.46-3.38 (m, 2H), 3.30 (s, 3H), 2.70-2.60 (m, 1H), 2.37 (s, 3H), 2.24 (s, 3H), 2.22 (s, 3H), 2.17-2.12 (m, 1H), 2.05-1.95 (m, 3H), 1.90-1.84 (m, 2H), 1.75-1.69 (m, 3H).

Compound 81 NMR: ¹H NMR (600 MHz, CDCl₃) δ 12.04 (s, 1H), 7.03 (t, J=6.0 Hz, 1H), 6.94 (s, 1H), 5.94 (s, 1H), 4.65-4.56 (m, 2H), 4.52-4.44 (m, 3H), 4.37-4.29 (m, 1H), 4.28-4.24 (m, 1H), 4.19-4.16 (m, 1H), 3.74-3.72 (m, 1H), 3.60-3.55 (m, 2H), 3.32 (s, 3H), 3.24-3.19 (m, 1H), 2.38 (s, 3H), 2.24 (s, 3H), 2.19 (s, 3H), 2.08-2.05 (m, 1H), 1.95-1.86 (m, 2H), 1.82-1.76 (m, 2H), 1.74-1.60 (m, 3H).

### Example 46, Compound 82 Synthesis

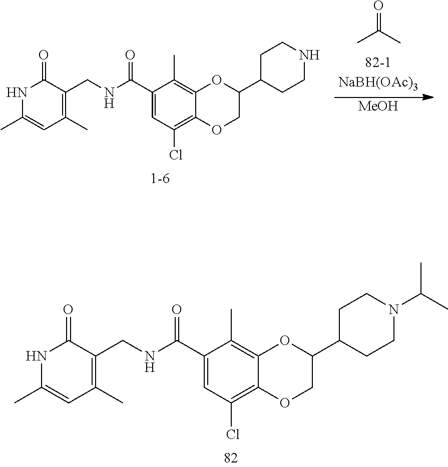

### Step 1, Compound 82 Synthesis

Following the synthesis method of Example 6, Step 4, except replacing Step 4, 6-3 with 1-6 (50.0 mg, 0.1 mmol), dimethylamine with 82-1 (20.0 mg, 0.3 mmol), DCE with MeOH, the same synthesis method was applied, to give Compound 82 (5.0 mg, 10.2 mol, 9.1% yield), LCMS (ESI⁺) m/z: 488.2 [M+H]⁺.

¹H NMR (400 MHz, DMSO-d₆) δ 11.47 (s, 1H), 8.15 (t, J=4.8 Hz, 1H), 6.92 (m, 1H), 5.86 (s, 1H), 4.46-4.44 (m, 1H), 4.24-4.21 (m, 2H), 4.17-4.12 (m, 1H), 4.10-4.03 (m, 1H), 3.47-3.41 (m, 2H), 3.03-2.90 (m, 3H), 2.17 (s, 3H), 2.14 (s, 3H), 2.11 (s, 3H), 2.10-2.01 (m, 1H), 1.96-1.91 (m, 2H), 1.74-1.59 (m, 2H), 1.25-1.15 (m, 6H).

### Example 47, Compound 83 Synthesis

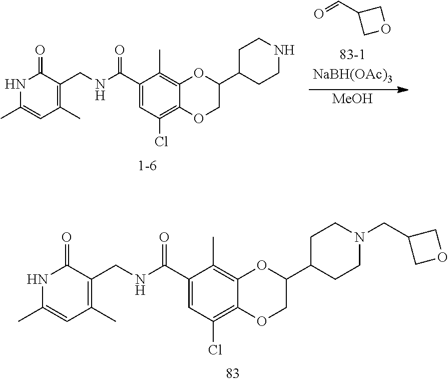 [SMILES: "MeOH" ; O=CC1COC1 ; Cc1cc(C)c(CNC(=O)c2cc(Cl)c3c(c2C)OC(C2CCNCC2)CO3)c(=O)[nH]1 ; Cc1cc(C)c(CNC(=O)c2cc(Cl)c3c(c2C)OC(C2CCN(CC4COC4)CC2)CO3)c(=O)[nH]1]

### Step 1, Compound 83 Synthesis

Following the synthesis method of Example 6, Step 4, except replacing Step 4, 6-3 with 1-6 (20.0 mg, 0.04 mmol), dimethylamine with 83-1 (8.0 mg, 0.09 mmol), DCE with MeOH, the same synthesis method was applied, to give Compound 83 (5.0 mg, 9.7 mol, 21.6% yield), LCMS (ESI⁺) m/z: 516.2 [M+H]⁺.

¹H NMR (400 MHz, DMSO-d₆) δ 11.46 (s, 1H), 8.12 (t, J=5.0 Hz, 1H), 6.89 (d, J=5.0 Hz, 1H), 5.85 (s, 1H), 4.62 (dd, J=7.8, 5.8 Hz, 2H), 4.23 (q, J=5.4, 4.8 Hz, 4H), 2.81 (s, 2H), 2.61 (s, 1H), 2.17 (s, 3H), 2.11 (s, 6H), 1.84 (d, J=9.2 Hz, 3H), 1.61 (s, 2H), 1.42-1.35 (m, 3H), 1.31-1.23 (m, 4H).

### Example 48, Compound 84 Synthesis

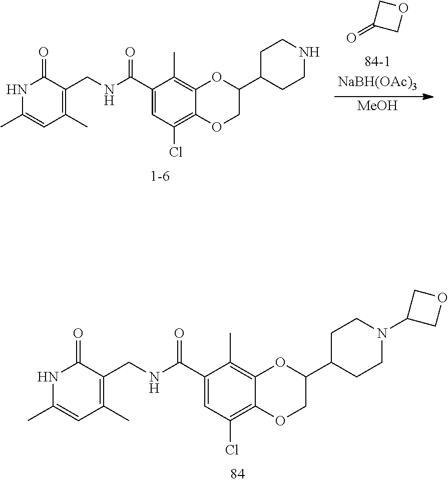 [SMILES: "MeOH" ; O=C1COC1 ; Cc1cc(C)c(CNC(=O)c2cc(Cl)c3c(c2C)OC(C2CCNCC2)CO3)c(=O)[nH]1 ; Cc1cc(C)c(CNC(=O)c2cc(Cl)c3c(c2C)OC(C2CCN(C4COC4)CC2)CO3)c(=O)[nH]1]

### Step 1, Compound 84 Synthesis

Following the synthesis method of Example 6, Step 4, except replacing Step 4, 6-3 with 1-6 (50.0 mg, 0.1 mmol), dimethylamine with 84-1 (16.0 mg, 0.2 mmol), DCE with MeOH, the same synthesis method was applied, to give Compound 84 (6.0 mg, 12.0 mol, 10.7% yield), LCMS (ESI⁺) m/z: 502.3 [M+H]⁺.

¹H NMR (400 MHz, Methanol-d₄) δ 6.96-6.91 (m, 1H), 6.09 (s, 1H), 4.42 (s, 2H), 3.98-3.96 (m, 1H), 2.36 (s, 3H), 2.34-2.29 (m, 6H), 2.24 (m, 3H), 2.21-2.18 (m, 3H), 2.11-1.96 (m, 1H), 1.89-1.72 (m, 3H), 1.71-1.51 (m, 3H), 1.41-1.36 (m, 2H), 1.20-1.19 (m, 1H).

### Example 49, Compound 85 Synthesis

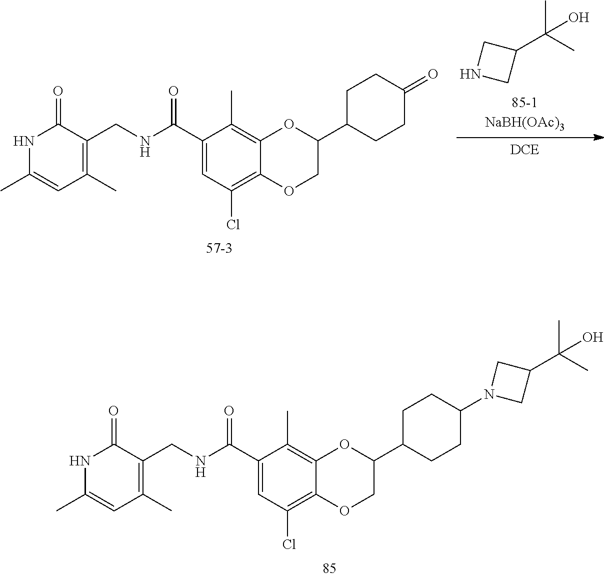

### Step 1, Compound 85 Synthesis

Following the synthesis method of Example 6, Step 4, except replacing Step 4, 6-3 with 57-3 (80.0 mg, 0.17 mmol), dimethylamine with 85-1 (80.0 mg, 0.7 mmol), the same synthesis method was applied. The obtained compound was resolved by SFC to give 85 (60.0 mg, 0.1 mmol, 61.8% yield), LCMS (ESI⁺) m/z: 558.3 [M+H]⁺.

¹H NMR (600 MHz, DMSO-d₆) δ 11.48 (s, 1H), 8.13 (t, J=4.8 Hz, 1H), 6.87 (s, 1H), 5.85 (s, 1H), 4.42 (dd, J=11.4, 1.8 Hz, 1H), 4.26-4.18 (m, 3H), 4.08-4.03 (m, 1H), 3.95-3.90 (m, 1H), 3.14-3.06 (m, 2H), 2.88-2.82 (m, 2H), 2.32-2.28 (m, 1H), 2.21-2.18 (m, 4H), 2.14-2.09 (m, 6H), 1.61-1.45 (m, 6H), 1.38-1.32 (m, 1H), 1.31-1.22 (m, 2H), 1.01 (m, 6H).

### Example 50, Compound 86 and 87 Synthesis

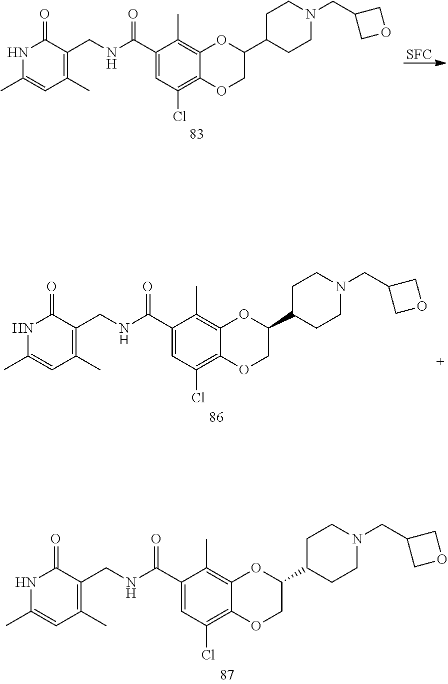

### Step 1, Compound 86 and 87 Synthesis

Compound 86 and 87 was obtained by SFC resolution of Compound 83. LCMS (ESI⁺) m/z: 516.2 [M+H]⁺, 538.1 [M+Na]⁺

Compound 86 NMR: ¹H NMR (400 MHz, DMSO-d₆) δ 11.49 (s, 1H), 8.13-8.11 (m, 1H), 6.90-6.87 (m, 1H), 5.75 (s, 1H), 4.65-4.62 (m, 2H), 4.26-4.21 (m, 4H), 2.83-2.78 (m, 2H), 2.63-2.58 (m, 1H), 2.17 (s, 3H), 2.11 (s, 6H), 1.90-1.80 (m, 3H), 1.64-1.58 (m, 2H), 1.42-1.35 (m, 3H), 1.38-1.35 (m, 4H).

Compound 87 NMR: ¹H NMR (400 MHz, DMSO-d₆) δ 11.49 (s, 1H), 8.13-8.11 (m, 1H), 6.90-6.87 (m, 1H), 5.75 (s, 1H), 4.65-4.62 (m, 2H), 4.26-4.21 (m, 4H), 2.83-2.78 (m, 2H), 2.63-2.58 (m, 1H), 2.17 (s, 3H), 2.11 (s, 6H), 1.90-1.80 (m, 3H), 1.64-1.58 (m, 2H), 1.42-1.35 (m, 3H), 1.38-1.35 (m, 4H).

### Example 51, Compound 88 and 89 Synthesis

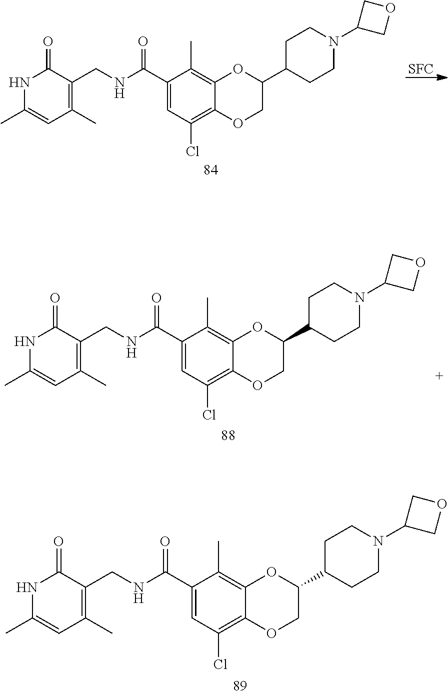

### Step 1, Compound 88 and 89 Synthesis

Compound 88 and 89 was obtained by SFC resolution of Compound 84.

Compound 88 NMR: ¹H NMR (400 MHz, methanol-d₄) δ 6.95-6.91 (m, 1H), 6.09 (s, 1H), 4.43-4.41 (m, 2H), 4.42-3.94 (m, 1H), 2.36 (s, 3H), 2.38-2.27 (m, 6H), 2.23 (s, 3H), 2.22-2.18 (m, 3H), 2.12-1.95 (m, 1H), 1.89-1.72 (m, 3H), 1.72-1.54 (m, 3H), 1.39-1.36 (s, 2H), 1.21-1.19 (m, 1H).

Compound 89 NMR: ¹H NMR (400 MHz, DMSO-d₆) δ 11.46 (m, 1H), 8.13 (t, J=4.8 Hz, 1H), 6.89 (s, 1H), 5.85 (s, 1H), 4.51-4.50 (m, 2H), 4.44-4.39 (m, 3H), 4.22-4.21 (m, 2H), 4.10-3.98 (m, 2H), 2.74-2.72 (m, 1H), 2.17 (s, 3H), 2.12 (s, 3H), 2.11 (s, 3H), 1.94-1.84 (m, 4H), 1.61-1.51 (m, 5H).

### Example 52, Compound 90 Synthesis

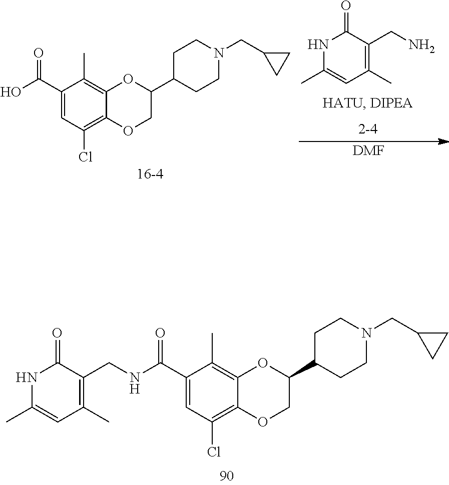

### Step 1, Compound 90 Synthesis

Following the synthesis method of Example 3, Step 1, except replacing Step 1, 2-3 with 16-4 (50.0 mg, 0.14 mmol), 3-1 with 2-4 (25.0 mg, 0.2 mmol), the same synthesis method was applied, to give Compound 90 (15.0 mg, 0.03 mmol, 22.0% yield), LCMS (ESI⁺) m/z: 500.3 [M+H]⁺.

¹H NMR (400 MHz, DMSO-d₆) δ 11.46 (s, 1H), 8.13 (t, J=5.2 Hz, 1H), 6.88 (s, 1H), 5.85 (s, 1H), 4.47 (dd, J=11.2, 2.0 Hz, 1H), 4.23 (d, J=4.8 Hz, 2H), 4.10-4.05 (m, 1H), 3.99-3.95 (m, 1H), 3.01 (d, J=11.2 Hz, 2H), 2.17 (s, 3H), 2.15-2.13 (m, 2H), 2.12-2.11 (s, 6H), 1.87-1.82 (m, 3H), 1.66-1.62 (m, 2H), 1.43-1.40 (m, 2H), 0.85-0.75 (m, 1H), 0.45-0.42 (m, 2H), 0.05-0.01 (m, 2H).

### Example 53, Compound 91 Synthesis

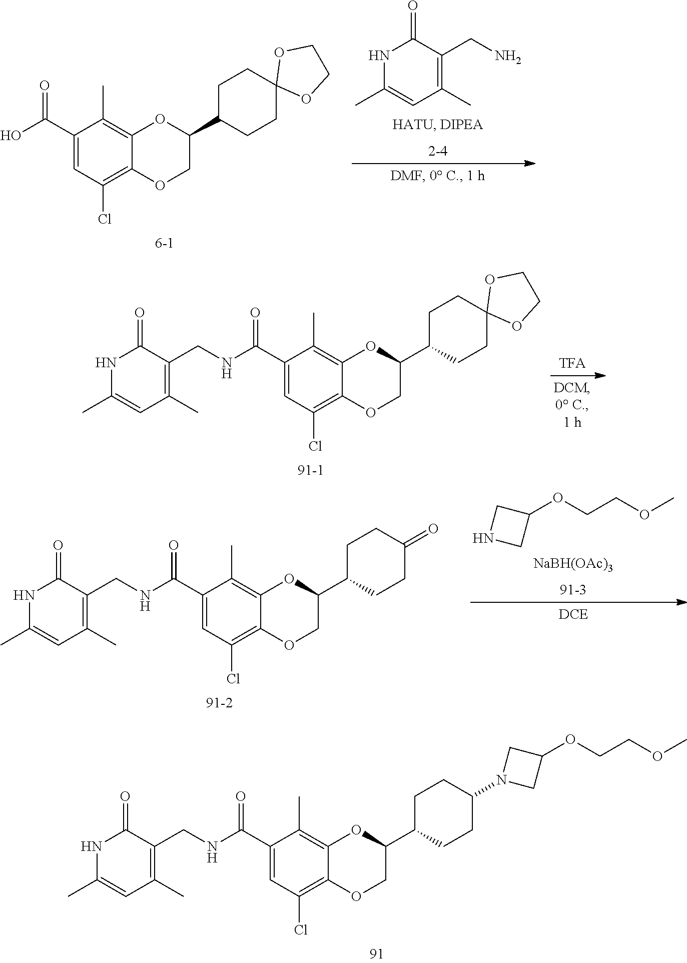

### Step 1, Compound 91-1 Synthesis

Following the synthesis method of Example 3, Step 1, except replacing Step 1, 2-3 with 6-1 (250.0 mg, 0.7 mmol), the same synthesis method was applied, to give Compound 91-1 (300.0 mg, 0.6 mmol), LCMS (ESI⁺) m/z: 503.2 [M+H]⁺.

### Step 2, Compound 91-2 Synthesis

Following the synthesis method of Example 3, Step 2, except replacing Step 2, 3-2 with 91-1 (300.0 mg, 0.6 mmol), the same synthesis method was applied, to give Compound 91-2 (250.0 mg, crude), LCMS (ESI⁺) m/z: 459.2 [M+H]⁺.

### Step 3, Compound 91 Synthesis

Following the synthesis method of Example 6, Step 4, except replacing Step 4, 6-3 with 91-2 (50.0 mg, 0.1 mmol), dimethylamine with 91-3 (43.0 mg, 0.3 mmol), the same synthesis method was applied. The obtained compound was subjected to Prep-MPLC to give Compound 91 (10.0 mg, 17.4 mol, 17.4% yield), LCMS (ESI⁺) m/z: 574.2 [M+H]⁺.

¹H NMR (400 MHz, DMSO-d₆) δ 11.46 (s, 1H), 8.12 (t, J=5.0 Hz, 1H), 6.87 (s, 1H), 5.85 (s, 1H), 4.45 (dd, J=11.4, 2.0 Hz, 1H), 4.22 (d, J=5.0 Hz, 2H), 4.07-4.02 (m, 1H), 3.99-3.97 (m, 1H), 3.93-3.91 (m, 1H), 3.45-3.42 (m, 6H), 3.23 (s, 3H), 2.73-2.70 (m, 2H), 2.17 (s, 3H), 2.11 (s, 6H), 1.98-1.84 (m, 2H), 1.77-1.71 (m, 3H), 1.59-1.48 (m, 1H), 1.19-1.13 (m, 2H), 0.89-0.83 (m, 2H).

### Example 54, Compound 92 Synthesis

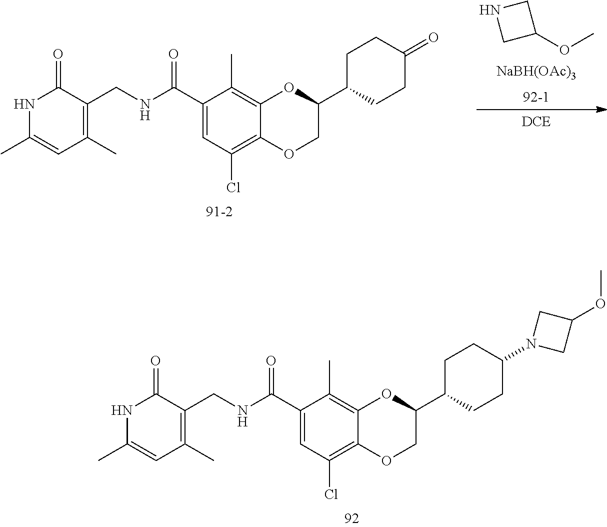

### Step 1, Compound 92 Synthesis

Following the synthesis method of Example 6, Step 4, except replacing Step 4, 6-3 with 91-2 (50.0 mg, 0.1 mmol), dimethylamine with 92-1 (28.0 mg, 0.3 mmol), the same synthesis method was applied. The obtained compound was subjected to Prep-MPLC to give Compound 92 (8.0 mg, 15.1 mol, 13.8% yield), LCMS (ESI⁺) m/z: 530.2 [M+H]⁺.

¹H NMR (400 MHz, DMSO-d₆) δ 11.46 (s, 1H), 8.13 (t, J=5.0 Hz, 1H), 6.88 (s, 1H), 5.85 (s, 1H), 4.46 (dd, J=11.4, 2.2 Hz, 1H), 4.22 (d, J=5.0 Hz, 2H), 4.07-4.02 (m, 1H), 3.92-3.87 (m, 2H), 3.45 (t, J=6.6 Hz, 2H), 3.13 (s, 3H), 2.72 (t, J=6.6 Hz, 2H), 2.17 (s, 3H), 2.11 (s, 6H), 1.96-1.88 (m, 2H), 1.78-1.70 (m, 3H), 1.57-1.49 (m, 1H), 1.24-1.10 (m, 2H), 0.94-0.81 (m, 2H).

### Example 55, Compound 93 Synthesis

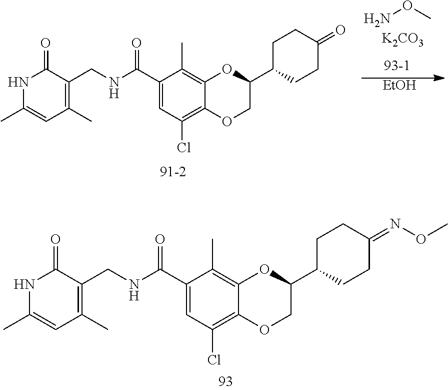

### Step 1, Compound 92 Synthesis

A single-necked flask was added with substrate 91-2 (20.0 mg, 44.0 mol). The mixture was dissolved with EtOH (5 mL) to dissolve, and then added with 93-1 (4.0 mg, 88.0 mol) and potassium carbonate (18.0 mg, 0.13 mmol), at 80° C. reacted for 2 hours, with LC-MS monitoring. After the completion of the reaction, and condensated under reduced pressure, and then subjected to Prep-MPLC preparation to give the product 92 (1.1 mg, 2.3 mol, 5.1% yield). LCMS (ESI⁺) m/z: 488.1 [M+H]⁺.

¹H NMR (400 MHz, DMSO-d₆) δ 11.46 (s, 1H), 8.12 (s, 1H), 6.89 (s, 1H), 5.85 (s, 1H), 4.47 (d, J=10.6 Hz, 1H), 4.23 (d, J=5.0 Hz, 2H), 4.16-3.92 (m, 2H), 3.71 (s, 3H), 3.06 (m, 1H), 2.33 (m, 1H), 2.29 (s, 1H), 2.17 (s, 3H), 2.14-2.09 (m, 6H), 2.08-2.07 (m, 1H) 1.93-1.78 (m, 3H), 1.33 (m, 2H).

### Example 56, Compound 94 Synthesis

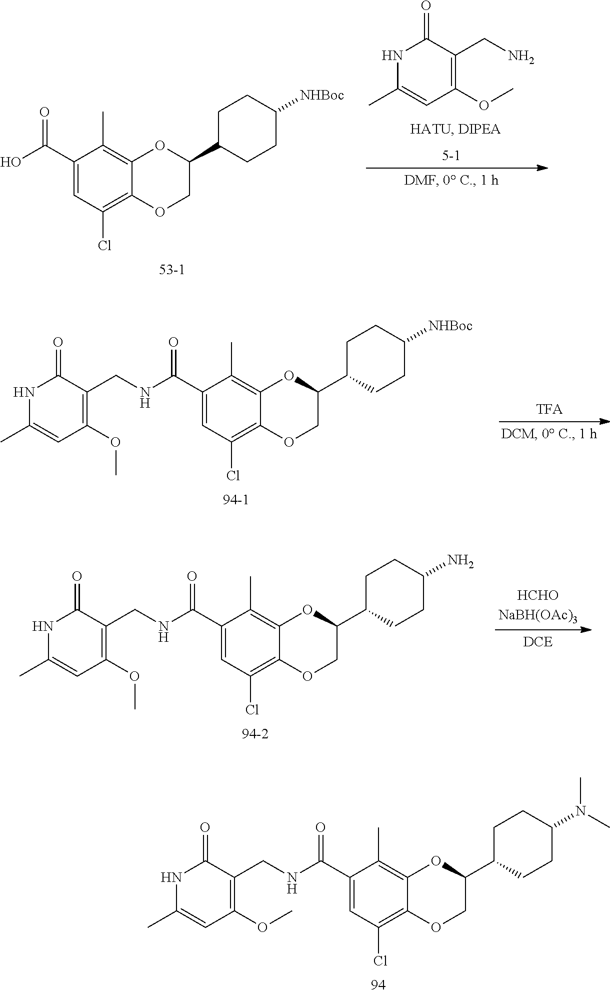

### Step 1, Compound 94-1 Synthesis

Following the synthesis method of Example 3, Step 1, except replacing Step 1, 2-3 with 53-1 (250.0 mg, 0.6 mmol), 2-4 with 5-1 (252.0 mg, 1.5 mmol), the same synthesis method was applied, to give Compound 94-1 (300.0 mg, 0.5 mmol), LCMS (ESI⁺) m/z: 576.2 [M+H]⁺.

### Step 2, Compound 94-2 Synthesis

Following the synthesis method of Example 3, Step 2, except replacing Step 2, 3-2 with 94-1 (300.0 mg, 0.5 mmol), the same synthesis method was applied, to give Compound 94-2 (250.0 mg, crude), LCMS (ESI⁺) m/z: 476.2 [M+H]⁺.

### Step 3, Compound 94 Synthesis

Following the synthesis method of Example 6, Step 4, except replacing Step 4, 6-3 with 94-2 (50.0 mg, 0.1 mmol), the same synthesis method was applied. The obtained compound was subjected to Prep-MPLC to give Compound 94 (10.0 mg, 0.02 mmol, 19.8% yield), LCMS (ESI⁺) m/z: 504.3 [M+H]⁺. 1H NMR (600 MHz, Methanol-d₄) δ 6.91 (s, 1H), 6.25 (s, 1H), 4.42-4.40 (m, 1H), 4.40 (s, 2H), 4.06-4.03 (m, 1H), 3.92 (s, 3H), 3.89-3.86 (m, 1H), 2.31 (s, 3H), 2.30 (s, 6H), 2.18 (s, 3H), 2.17-2.15 (m, 1H), 2.09 (m, 2H), 1.89-1.88 (m, 1H), 1.63-1.61 (m, 1H), 1.32-1.25 (m, 5H).

### Example 57, Compound 95 and 96 Synthesis

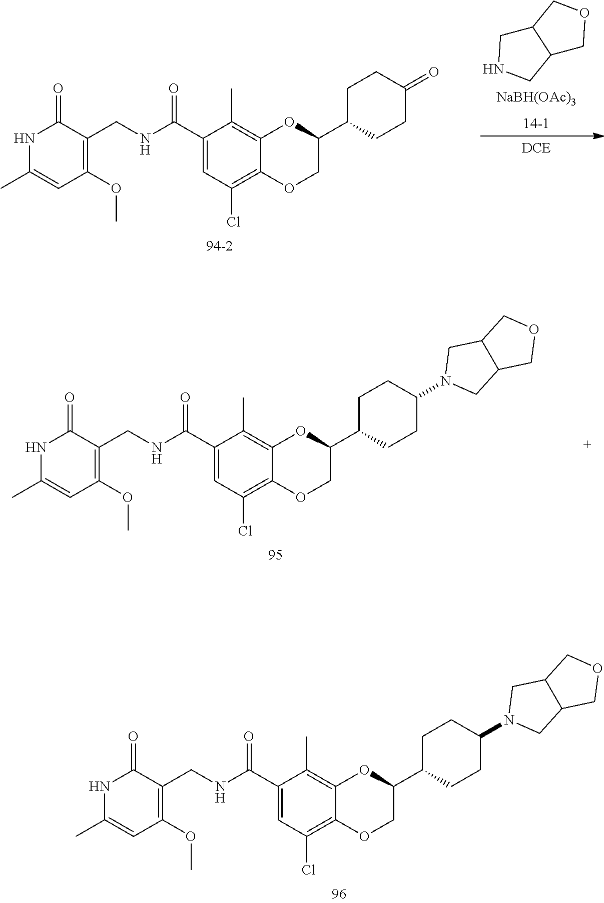

### Step 1, Compound 95 and 96 Synthesis

Following the synthesis method of Example 6, Step 4, except replacing Step 4, 6-3 with 94-2 (1.3 g, 2.7 mmol), dimethylamine with 14-1 (929.0 mg, 8.0 mmol), the same synthesis method was applied. The obtained compound was subjected to Prep-MPLC to give Compound 95 (64.0 mg, 0.1 mmol, 4.1% yield) and 96 (190.0 mg, 0.3 mmol, 12.3% yield), LCMS (ESI⁺) m/z: 572.3 [M+H]⁺.

Compound 95 NMR: ¹H NMR (400 MHz, Methanol-d₄) δ 6.92 (s, 1H), 6.25 (s, 1H), 4.42-4.40 (m, 1H), 4.39 (s, 2H), 4.08-4.03 (m, 1H), 3.92 (s, 3H), 3.90-3.86 (m, 1H), 3.70-3.62 (m, 4H), 3.20-3.18 (m, 2H), 2.88-2.84 (m, 2H), 2.31 (s, 3H), 2.29-2.25 (m, 1H), 2.18 (s, 3H), 2.12-2.10 (m, 4H), 1.87-1.83 (m, 1H), 1.66-1.60 (m, 1H), 1.33-1.26 (m, 5H).

Compound 96 NMR: ¹H NMR (400 MHz, Methanol-d₄) δ 6.91 (s, 1H), 6.25 (s, 1H), 4.46-4.42 (m, 1H), 4.40 (s, 2H), 4.07-4.02 (m, 2H), 3.92 (s, 3H), 3.83-3.79 (m, 2H), 3.56-3.52 (m, 2H), 2.81-2.77 (m, 2H), 2.72-2.68 (m, 2H), 2.43-2.39 (m, 2H), 2.31 (s, 3H), 2.18 (s, 3H), 2.17-2.13 (m, 1H), 1.87-1.68 (m, 5H), 1.65-1.50 (m, 4H)

### Example 58, Compound 97 and 98 Synthesis

### Step 1, Compound 97 and 98 Synthesis

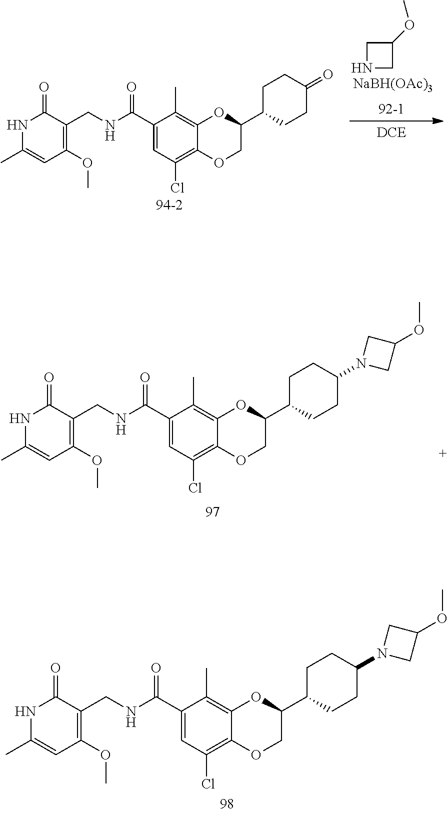

Following the synthesis method of Example 6, Step 4, except replacing Step 4, 6-3 with 94-2 (1.0 g, 2.1 mmol), dimethylamine with 92-1 (220.0 mg, 2.5 mmol), the same synthesis method was applied. The obtained compound was subjected to Prep-MPLC to give Compound 97 (90.0 mg, 0.2 mmol, 7.8% yield) and 98 (180.0 mg, 0.3 mmol, 15.7% yield), LCMS (ESI⁺) m/z: 546.6 [M+H]⁺.

Compound 97 NMR: ¹H NMR (400 MHz, DMSO-d₆) δ 11.42 (s, 1H), 7.93 (t, J=4.4 Hz, 1H), 6.87 (s, 1H), 6.09 (s, 1H), 4.46 (dd, J=11.6, 2.0 Hz, 1H), 4.17 (d, J=4.4 Hz, 2H), 4.06-4.02 (m, 1H), 3.92-3.87 (m, 2H), 3.79 (s, 3H), 3.45 (t, J=7.6 Hz, 2H), 3.13 (s, 3H), 2.73 (t, J=6.0 Hz, 2H), 2.18 (s, 3H), 2.12 (s, 3H), 1.99-1.83 (m, 2H), 1.78-1.74 (m, 3H), 1.55-1.53 (m, 1H), 1.18-1.15 (m, 2H), 0.89-0.86 (m, 2H).

Compound 98 NMR: ¹H NMR (600 MHz, DMSO-d₆) δ 11.42 (s, 1H), 7.93 (t, J=4.2 Hz, 1H), 6.87 (s, 1H), 6.09 (s, 1H), 4.46 (dd, J=12.0, 2.4 Hz, 1H), 4.17 (d, J=4.8 Hz, 2H), 4.06-4.02 (m, 1H), 3.92-3.87 (m, 2H), 3.79 (s, 3H), 3.45 (t, J=6.6 Hz, 2H), 3.31-3.29 (m, 1H), 3.13 (s, 3H), 2.72 (t, J=6.0 Hz, 2H), 2.18 (s, 3H), 2.12 (s, 3H), 1.95-1.93 (m, 1H), 1.88-1.87 (m, 1H), 1.77-1.75 (m, 2H), 1.56-1.51 (m, 1H), 1.20-1.12 (m, 2H), 0.91-0.83 (m, 2H).

### Example 59, Compound 99 and 100 Synthesis

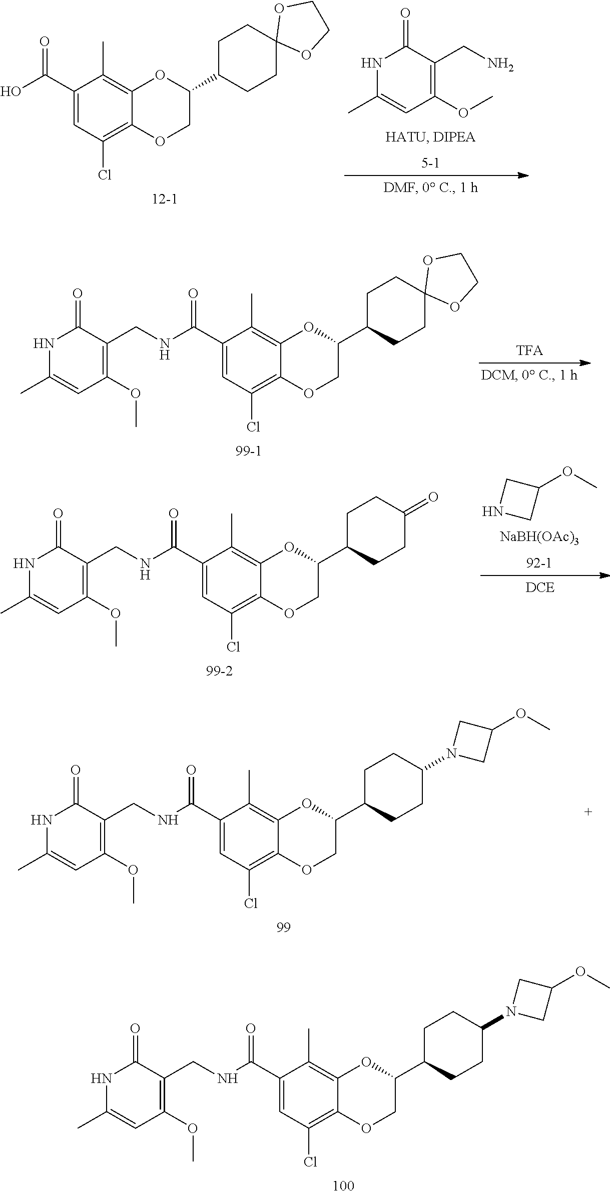

### Step 1, Compound 99-1 Synthesis

Following the synthesis method of Example 3, Step 1, except replacing Step 1, 2-3 with 12-1 (250.0 mg, 0.7 mmol), 3-1 with 5-1 (252.0 mg, 1.5 mmol) the same synthesis method was applied, to give Compound 99-1 (300.0 mg, 0.6 mmol), LCMS (ESI⁺) m/z: 519.1 [M+H]⁺.

### Step 2, Compound 99-2 Synthesis

Following the synthesis method of Example 3, Step 2, except replacing Step 2, 3-2 with 99-1 (300.0 mg, 0.6 mmol), the same synthesis method was applied, to give Compound 99-2 (250.0 mg, crude), LCMS (ESI⁺) m/z: 475.2 [M+H]⁺.

### Step 3, Compound 99 and 100 Synthesis

Following the synthesis method of Example 6, Step 4, except replacing Step 4, 6-3 with 99-2 (200.0 mg, 0.4 mmol), dimethylamine with 92-1 (174.0 mg, 2.0 mmol), the same synthesis method was applied. The obtained compound was subjected to Prep-MPLC to give Compound 99 (20.0 mg, 0.04 mmol, 9.2% yield) and 100 (22.0 mg, 0.04 mmol, 10.1% yield), LCMS (ESI⁺) m/z: 546.6 [M+H]⁺.

Compound 99 NMR: ¹H NMR (600 MHz, DMSO-d₆) δ 11.42 (s, 1H), 7.92 (t, J=4.8 Hz, 1H), 6.86 (s, 1H), 6.09 (s, 1H), 4.43 (dd, J=11.4, 2.4 Hz, 1H), 4.17 (d, J=4.8 Hz, 2H), 4.06-4.04 (m, 1H), 3.93-3.91 (m, 2H), 3.79 (s, 3H), 3.47-3.43 (m, 2H), 3.14 (s, 3H), 2.68 (t, J=6.0 Hz, 2H), 2.21-2.20 (m, 1H), 2.18 (s, 3H), 2.11 (s, 3H), 1.60-1.56 (m, 4H), 1.50-1.48 (m, 2H), 1.44-1.42 (m, 1H), 1.38-1.30 (m, 2H).

Compound 100 NMR: ¹H NMR (600 MHz, DMSO-d₆) δ 11.42 (s, 1H), 7.92 (t, J=4.8 Hz, 1H), 6.86 (s, 1H), 6.09 (s, 1H), 4.43 (dd, J=11.4, 2.2 Hz, 1H), 4.17 (d, J=4.8 Hz, 2H), 4.08-4.04 (m, 1H), 3.93-3.89 (m, 2H), 3.79 (s, 3H), 3.49-3.42 (m, 2H), 3.14 (s, 3H), 2.68 (t, J=6.0 Hz, 2H), 2.21-2.19 (m, 1H), 2.18 (s, 3H), 2.11 (s, 3H), 1.60-1.56 (m, 4H), 1.50-1.48 (m, 2H), 1.44-1.42 (m, 1H), 1.38-1.30 (m, 2H).

### Example 60, Compound 101 and 102 Synthesis

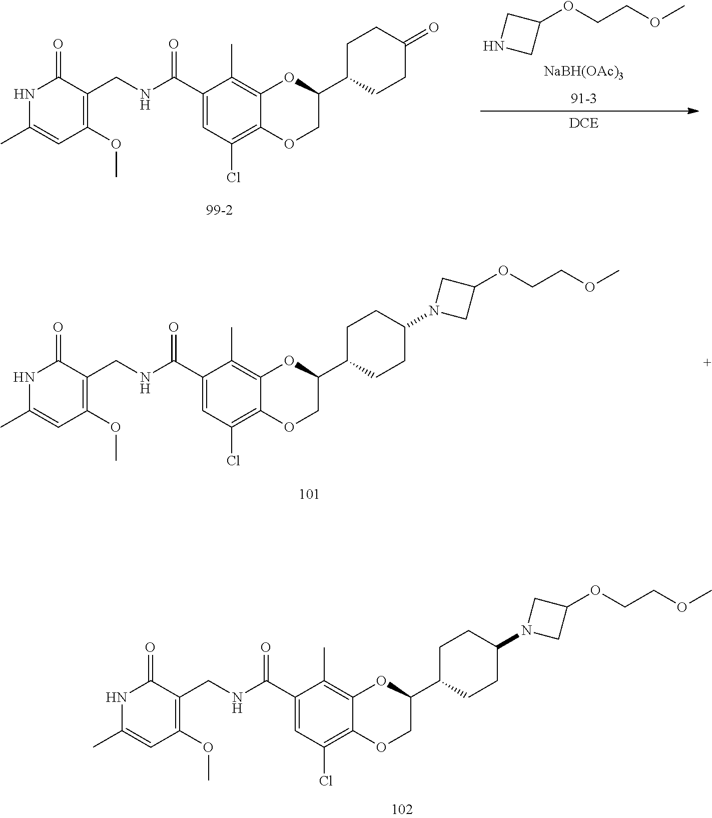

### Step 1, Compound 101 and 102 Synthesis

Following the synthesis method of Example 6, Step 4, except replacing Step 4, 6-3 with 99-2 (20.0 mg, 0.04 mmol), dimethylamine with 91-3 (17.0 mg, 0.1 mmol), the same synthesis method was applied. The obtained compound was subjected to Prep-MPLC to give Compound 101 (3.0 mg, 5.0 mol, 12.1% yield) and 102 (3.0 mg, 5.0 mol, 12.1% yield), LCMS (ESI⁺) m/z: 590.2 [M+H]⁺.

Compound 101 NMR: ¹H NMR (400 MHz, DMSO-d₆) δ 11.42 (s, 1H), 7.93 (t, J=4.8 Hz, 1H), 6.87 (s, 1H), 6.09 (s, 1H), 4.46 (dd, J=11.4, 2.2 Hz, 1H), 4.18 (d, J=4.6 Hz, 2H), 4.07-4.02 (m, 2H), 4.00-3.91 (m, 1H), 3.79 (s, 3H), 3.46-3.45 (m, 5H), 3.24-3.23 (m, 4H), 2.71-2.73 (m, 2H), 2.18 (s, 3H), 2.12 (s, 3H), 1.92-1.77 (m, 2H), 1.19-1.13 (m, 3H), 1.54-1.50 (m, 1H), 1.21-1.12 (m, 2H), 0.89-0.84 (m, 2H).

Compound 102 NMR: ¹H NMR (400 MHz, DMSO-d₆) δ 11.42 (s, 1H), 7.93 (t, J=4.4 Hz, 1H), 6.87 (s, 1H), 6.09 (s, 1H), 4.46 (dd, J=11.4, 2.2 Hz, 1H), 4.18 (d, J=4.6 Hz, 2H), 4.07-4.02 (m, 2H), 4.00-3.91 (m, 1H), 3.79 (s, 3H), 3.46-3.45 (m, 5H), 3.24-3.23 (m, 4H), 2.71-2.73 (m, 2H), 2.18 (s, 3H), 2.12 (s, 3H), 1.92-1.77 (m, 2H), 1.19-1.13 (m, 3H), 1.54-1.50 (m, 1H), 1.21-1.12 (m, 2H), 0.89-0.84 (m, 2H).

### Example 61, Compound 103 and 104 Synthesis

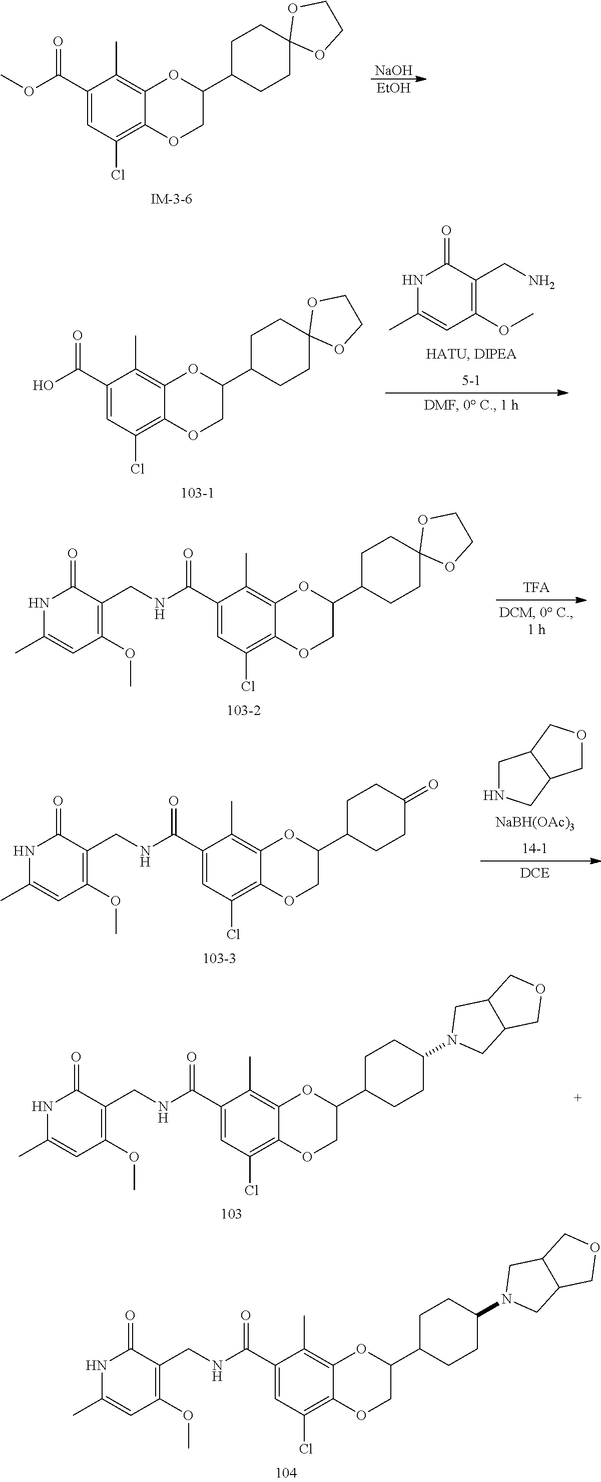

### Step 1, Compound 103-1 Synthesis

Following the synthesis method of Example 6, Step 1, except replacing Step 1, IM-3a with IM-3-6 (150.0 mg, 0.4 mmol), the same synthesis method was applied, to give Compound 103-1 (100 mg, crude), LCMS (ESI⁺) m/z: 369.1 [M+H]⁺.

### Step 2, Compound 103-2 Synthesis

Following the synthesis method of Example 3, Step 1, except replacing Step 1, 2-3 with 103-1 (100.0 mg, crude), 3-1 with 5-1 (68.5 mg, 0.4 mmol), the same synthesis method was applied, to give Compound 103-2 (85.0 mg, 0.2 mmol, 60.5% yield), LCMS (ESI⁺) m/z: 519.2 [M+H]⁺.

### Step 3, Compound 103-3 Synthesis

Following the synthesis method of Example 3, Step 2, except replacing Step 2, 3-2 with 103-2 (85.0 mg, 0.2 mmol), the same synthesis method was applied, to give Compound 103-3 (50.0 mg, crude), LCMS (ESI⁺) m/z: 475.2 [M+H]⁺.

### Step 4, Compound 103 and 104 Synthesis

Following the synthesis method of Example 6, Step 4, except replacing Step 4, dimethylamine with 14-1 (17.9 mg, 0.2 mmol), the same synthesis method was applied. The obtained compound was subjected to Prep-MPLC to give Compound 103 (10.0 mg, 17.5 mol, 17.5% yield) and 104 (10.0 mg, 17.5 mol, 17.5% yield), LCMS (ESI⁺) m/z: 572.2 [M+H]⁺.

Compound 103 NMR: ¹H NMR (400 MHz, DMSO-d₆) δ 11.42 (s, 1H), 7.93 (t, J=4.2 Hz, 1H), 6.87 (s, 1H), 6.09 (s, 1H), 4.44 (dd, J=11.4, 1.8 Hz, 1H), 4.17 (d, J=4.2 Hz, 2H), 4.08-4.05 (m, 1H), 3.97-3.95 (m, 1H), 3.86-3.85 (m, 2H), 3.79 (s, 3H), 3.30-3.28 (m, 2H), 2.67-2.65 (m, 2H), 2.48-2.46 (m, 2H), 2.32-2.31 (m, 2H), 2.18 (s, 3H), 2.15-2.13 (m, 1H), 2.11 (s, 3H), 1.80-1.78 (m, 2H), 1.67-1.58 (m, 4H), 1.42-1.40 (m, 3H).

Compound 104 NMR: ¹H NMR (400 MHz, DMSO-d₆) δ 11.42 (s, 1H), 7.93 (t, J=4.2 Hz, 1H), 6.87 (s, 1H), 6.09 (s, 1H), 4.46-4.44 (m, 1H), 4.17 (d, J=4.8 Hz, 2H), 4.05-4.02 (m, 1H), 3.96-3.90 (m, 1H), 3.79 (s, 3H), 3.70-3.69 (m, 2H), 3.30-3.28 (m, 2H), 2.68-2.62 (m, 4H), 2.36-2.34 (m, 2H), 2.18 (s, 3H), 2.12 (s, 3H), 1.96-1.91 (m, 4H), 1.75-1.73 (m, 1H), 1.57-1.55 (m, 1H), 1.22-1.11 (m, 4H).

### Example 62, Compound 105 and 106 Synthesis

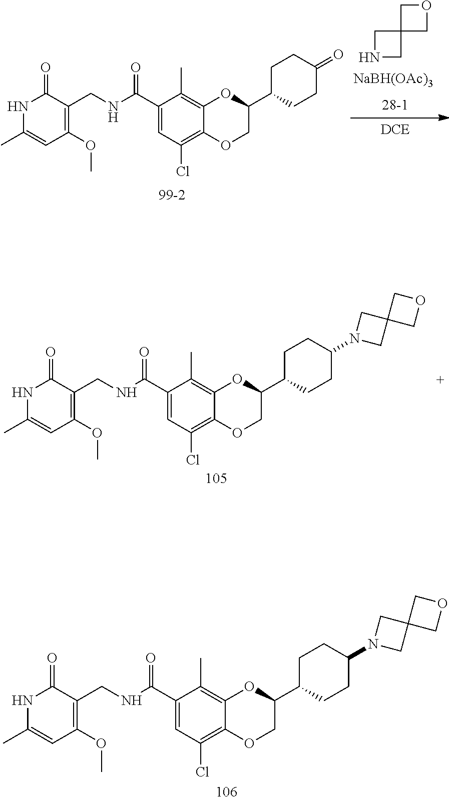

### Step 1, Compound 105 and 106 Synthesis

Following the synthesis method of Example 6, Step 4, except replacing Step 4, 6-3 with 99-2 (30.0 mg, 0.06 mmol), dimethylamine with 28-1 (12.5 mg, 0.1 mmol), the same synthesis method was applied. The obtained compound was subjected to Prep-MPLC to give Compound 105 (3.0 mg, 5.0 mol, 8.5% yield) and 106 (6.0 mg, 0.01 mmol, 17.0% yield), LCMS (ESI⁺) m/z: 558.1 [M+H]⁺.

Compound 105 NMR: ¹H NMR (600 MHz, DMSO-d₆) δ 11.42 (s, 1H), 7.93 (t, J=4.2 Hz, 1H), 6.87 (s, 1H), 6.09 (s, 1H), 4.60 (s, 4H), 4.43 (d, J=11.4 Hz, 1H), 4.17 (d, J=4.8 Hz, 2H), 4.07-4.04 (m, 1H), 3.97-3.95 (m, 1H), 3.79 (s, 3H), 3.25-3.15 (m, 4H), 2.57-2.54 (m, 1H), 2.18 (s, 3H), 2.11 (s, 3H), 1.60-1.49 (m, 5H), 1.46-1.33 (m, 4H).

Compound 106 NMR: ¹H NMR (400 MHz, DMSO-d₆) δ 11.42 (s, 1H), 7.93 (t, J=4.8 Hz, 1H), 6.87 (s, 1H), 6.09 (s, 1H), 4.57 (s, 4H), 4.45 (d, J=11.6 Hz, 1H), 4.17 (d, J=4.4 Hz, 2H), 4.06-4.01 (m, 1H), 3.92-3.89 (m, 1H), 3.79 (s, 3H), 3.24-3.19 (m, 4H), 2.44-2.41 (m, 1H), 2.18 (s, 3H), 2.11 (s, 3H), 1.95-1.91 (m, 2H), 1.83-1.80 (m, 1H), 1.73-1.71 (m, 4H), 1.50-1.52 (m, 2H).

### Example 63, Compound 107 and 108 Synthesis

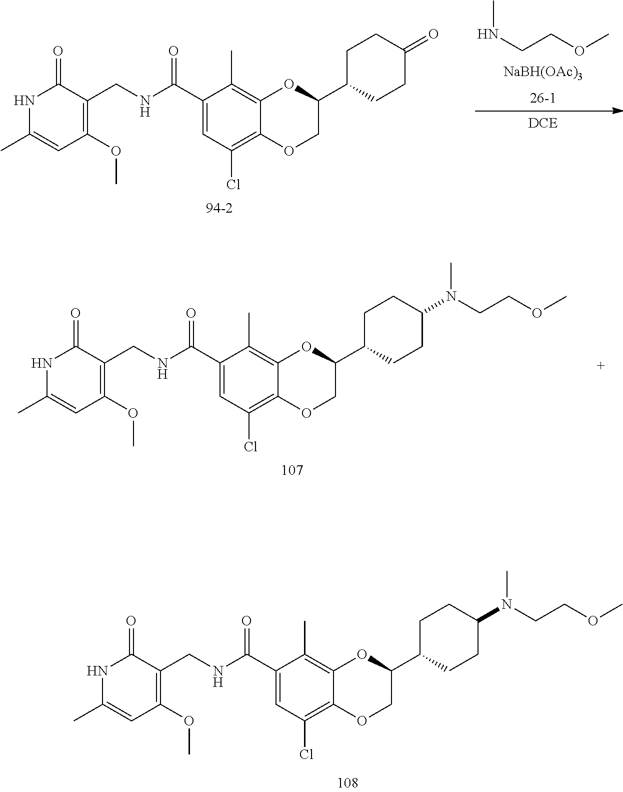

### Step 1, Compound 107 and 108 Synthesis

Following the synthesis method of Example 6, Step 4, except replacing Step 4, 6-3 with 94-2 (50.0 mg, 0.1 mmol), dimethylamine with 26-1 (19.0 mg, 0.2 mmol), the same synthesis method was applied. The obtained compound was subjected to Prep-MPLC to give Compound 107 (5.0 mg, 0.01 mmol, 8.7% yield) and 108 (1.0 mg, 2.0 mol, 1.7% yield), LCMS (ESI⁺) m/z: 548.3 [M+H]⁺.

Compound 107 NMR: ¹H NMR (400 MHz, CDCl₃) δ 11.08 (s, 1H), 7.30-7.26 (m, 1H), 7.00 (s, 1H), 5.95 (s, 1H), 4.53 (d, J=5.2 Hz, 2H), 4.35 (dd, J=11.2, 2.0 Hz, 1H), 4.05-4.02 (m, 1H), 3.89 (s, 3H), 3.87-3.83 (m, 2H), 3.76-3.49 (m, 1H), 3.49 (s, 2H), 3.37 (s, 3H), 3.13-3.11 (m, 3H), 2.69-2.66 (m, 3H), 2.31 (s, 3H), 2.23 (s, 3H), 2.19-2.16 (m, 1H), 1.94-1.93 (m, 1H), 1.88-1.81 (m, 1H), 1.68-1.64 (m, 2H), 1.52-1.42 (m, 2H).

Compound 108 NMR: ¹H NMR (400 MHz, CDCl₃) δ 12.16 (s, 1H), 7.37-7.35 (m, 1H), 7.00 (s, 1H), 5.96 (s, 1H), 4.52-4.50 (m, 2H), 4.43 (d, J=10.8 Hz, 1H), 4.26-4.24 (m, 1H), 4.08-4.06 (m, 1H), 4.05-4.03 (m, 1H), 3.98 (s, 3H), 3.94-3.86 (m, 1H), 3.37 (s, 3H), 3.33-3.32 (m, 2H), 3.20-3.18 (m, 1H), 2.79-2.77 (m, 3H), 2.32 (s, 3H), 2.23 (s, 3H), 2.09-2.04 (m, 1H), 1.94-1.93 (m, 2H), 1.85-1.80 (m, 3H), 1.73-1.68 (m, 2H), 1.56-1.54 (m, 1H).

### Example 64, Compound 109 and 110 Synthesis

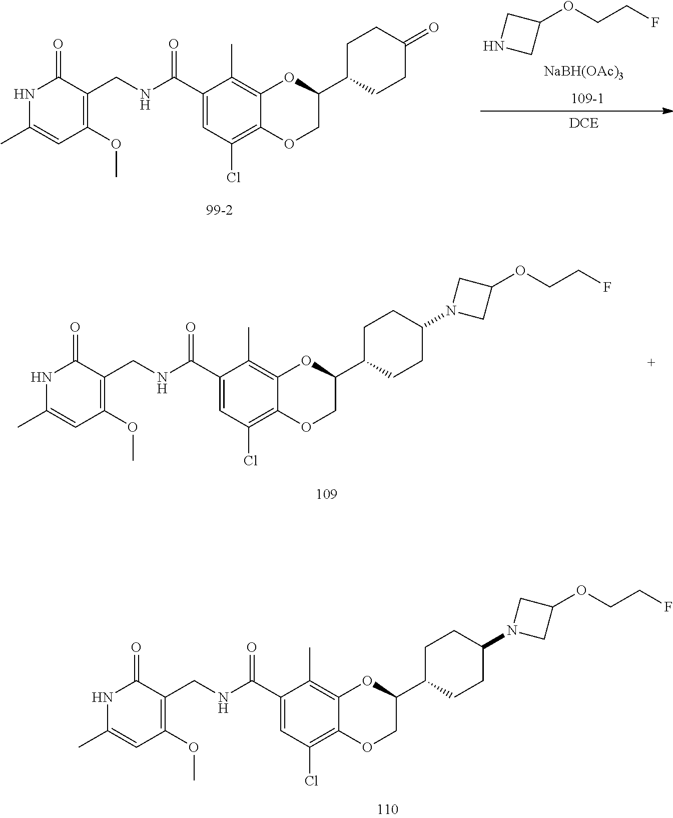

### Step 1, Compound 109 and 110 Synthesis

Following the synthesis method of Example 6, Step 4, except replacing Step 4, 6-3 with 99-2 (50.0 mg, 0.1 mmol), dimethylamine with 109-1 (25.0 mg, 0.2 mmol), the same synthesis method was applied. The obtained compound was subjected to Prep-MPLC to give Compound 109 (7.5 mg, 0.01 mmol, 12.3% yield) and 110 (4.6 mg, 8.0 mol, 7.6% yield), LCMS (ESI⁺) m/z: 578.3 [M+H]⁺.

Compound 109 NMR: ¹H NMR (400 MHz, CDCl₃) δ 11.07 (m, 1H), 7.33-7.32 (m, 1H), 7.00 (s, 1H), 5.95 (s, 1H), 4.59-4.58 (m, 1H), 4.57-4.54 (m, 2H), 4.53-4.47 (m, 1H), 4.46-4.45 (m, 1H), 4.37-4.34 (m, 1H), 4.03-4.01 (m, 1H), 4.00-3.98 (m, 1H), 3.97-3.95 (m, 3H), 3.92-3.89 (m, 1H), 3.87-3.85 (m, 1H), 3.76-3.70 (m, 1H), 3.68-3.66 (m, 1H), 2.30 (s, 3H), 2.24 (s, 3H), 2.13-2.12 (m, 1H), 2.05-2.04 (m, 2H), 1.94-1.89 (m, 1H), 1.83-1.69 (m, 3H), 1.28-1.25 (m, 4H), 0.90-0.84 (m, 1H).

Compound 110 NMR: ¹H NMR (400 MHz, CDCl₃) δ 12.04 (m, 1H), 7.33-7.32 (m, 1H), 7.00 (s, 1H), 6.09 (s, 1H), 4.61-4.59 (m, 2H), 4.58-4.57 (m, 2H), 4.53-4.47 (m, 2H), 4.46-4.45 (m, 2H), 4.45-4.35 (m, 2H), 4.25-4.21 (m, 1H), 4.17-4.15 (m, 1H), 3.96-3.94 (m, 3H), 3.77-3.76 (m, 2H), 3.70-3.66 (m, 4H), 3.26-3.24 (m, 1H), 2.45-2.39 (m, 3H), 2.25 (s, 3H), 2.22-2.04 (m, 4H), 0.98-0.95 (m, 1H).

### Example 65, Compound 111 and 112 Synthesis

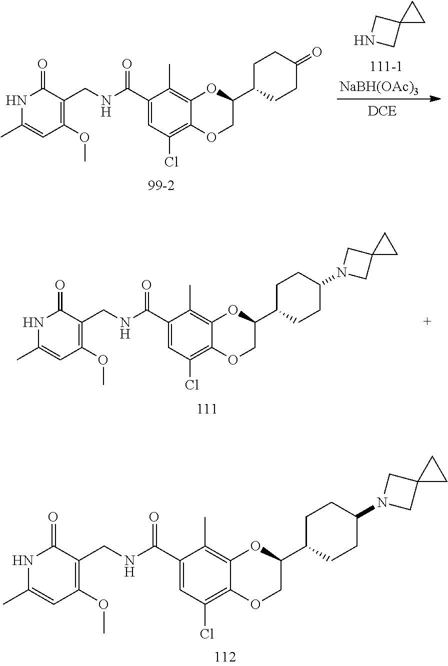

### Step 1, Compound 111 and 112 Synthesis

Following the synthesis method of Example 6, Step 4, except replacing Step 4, 6-3 with 99-2 (40.0 mg, 0.08 mmol), dimethylamine with 111-1 (14.0 mg, 0.2 mmol), the same synthesis method was applied. The obtained compound was subjected to Prep-MPLC to give Compound 111 (2.0 mg, 3.7 mol, 4.4% yield) and 112 (4.0 mg, 7.4 mol, 8.8% yield), LCMS (ESI⁺) m/z: 542.4 [M+H]⁺.

Compound 111 NMR: ¹H NMR (400 MHz, DMSO-d₆) δ 11.42 (s, 1H), 7.93 (t, J=4.4 Hz, 1H), 6.87 (s, 1H), 6.09 (s, 1H), 4.47-4.44 (m, 1H), 4.18 (d, J=4.8 Hz, 2H), 4.08-4.03 (m, 1H), 3.94-3.91 (m, 1H), 3.79 (s, 3H), 3.24-3.22 (m, 4H), 2.45-2.40 (m, 1H), 2.18 (s, 3H), 2.12 (s, 3H), 1.98-1.95 (m, 1H), 1.78-1.73 (m, 3H), 1.57-1.55 (m, 1H), 1.23-1.13 (m, 2H), 1.01-0.84 (m, 2H), 0.49 (s, 4H).

Compound 112 NMR: ¹H NMR (400 MHz, DMSO-d₆) δ 11.42 (s, 1H), 7.93 (t, J=4.4 Hz, 1H), 6.87 (s, 1H), 6.09 (s, 1H), 4.45-4.42 (m, 1H), 4.18 (d, J=4.4 Hz, 2H), 4.09-4.04 (m, 1H), 3.99-3.95 (m, 1H), 3.79 (s, 3H), 3.17 (s, 4H), 2.34-2.32 (m, 1H), 2.18 (s, 3H), 2.12 (s, 3H), 1.66-1.57 (m, 5H), 1.50-1.47 (m, 1H), 1.40-1.33 (m, 3H), 0.48 (s, 4H).

### Example 66, Compound 113 and 114 Synthesis

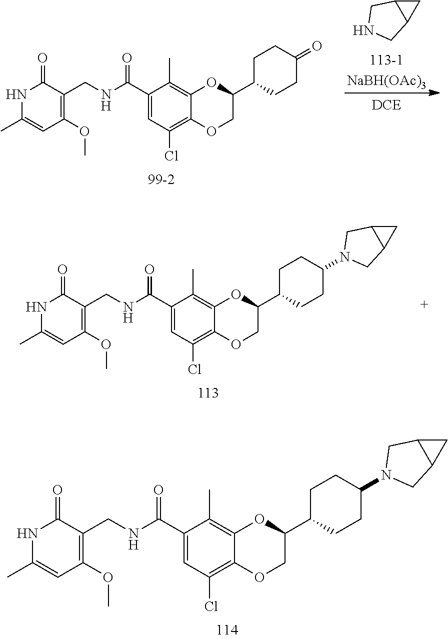

### Step 1, Compound 113 and 114 Synthesis

Following the synthesis method of Example 6, Step 4, except replacing Step 4, 6-3 with 99-2 (40.0 mg, 0.08 mmol), dimethylamine with 113-1 (14.0 mg, 0.2 mmol), the same synthesis method was applied. The obtained compound was subjected to Prep-MPLC to give Compound 113 (2.0 mg, 3.7 mol, 4.3% yield) and 114 (5.0 mg, 9.2 mol, 10.7% yield), LCMS (ESI⁺) m/z: 542.3 [M+H]⁺.

Compound 113 NMR: ¹H NMR (400 MHz, Methanol-d₄) δ 6.91 (s, 1H), 6.25 (s, 1H), 4.42-4.40 (m, 2H), 4.39-4.38 (m, 1H), 4.07-4.02 (m, 1H), 3.92 (s, 3H), 3.89-3.85 (m, 1H), 3.18-3.16 (m, 1H), 2.68-2.63 (m, 2H), 2.31 (s, 3H), 2.18 (s, 3H), 2.11-2.07 (m, 4H), 1.84-1.81 (m, 1H), 1.65-1.61 (m, 1H), 1.53-1.51 (m, 2H), 1.32-1.23 (m, 5H), 0.63-0.60 (m, 1H), 0.52-0.49 (m, 1H).

Compound 114 NMR: ¹H NMR (400 MHz, DMSO-d₆) δ 11.42 (s, 1H), 7.93 (t, J=4.8 Hz, 1H), 6.87 (s, 1H), 6.09 (s, 1H), 4.43 (d, J=11.6 Hz, 1H), 4.18 (d, J=4.8 Hz, 2H), 4.08-4.03 (m, 1H), 4.00-3.94 (m, 1H), 3.79 (s, 3H), 2.96 (d, J=8.4 Hz, 2H), 2.23-2.20 (m, 1H), 2.18 (s, 3H), 2.16-2.14 (m, 1H), 2.12 (s, 3H), 1.75-1.74 (m, 2H), 1.73-1.71 (m, 1H), 1.66-1.64 (m, 3H), 1.37-1.34 (m, 5H), 1.24-1.23 (m, 1H), 0.58-0.57 (m, 1H), 0.30-0.28 (m, 1H).

### Example 67, Compound 115 and 116 Synthesis

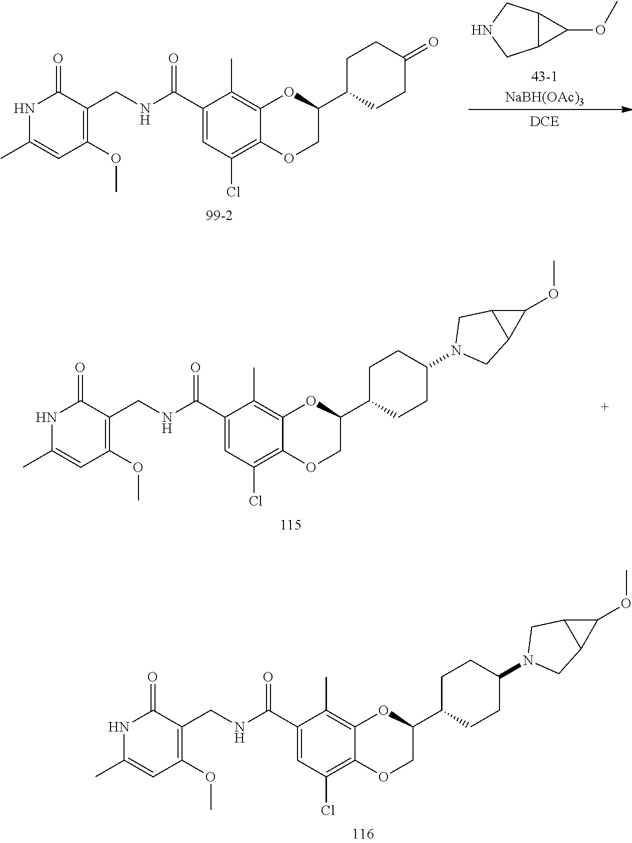

### Step 1, Compound 115 and 116 Synthesis

Following the synthesis method of Example 6, Step 4, except replacing Step 4, 6-3 with 99-2 (50.0 mg, 0.1 mmol), dimethylamine with 43-1 (11.6 mg, 0.1 mmol), the same synthesis method was applied. The obtained compound was subjected to Prep-MPLC to give Compound 115 (12.0 mg, 0.02 mmol, 19.9% yield) and 116 (11.0 mg, 19.2 mol, 18.3% yield), LCMS (ESI⁺) m/z: 572.3 [M+H]⁺.

Compound 115 NMR: ¹H NMR (600 MHz, CDCl₃) δ 6.98 (s, 1H), 5.98 (s, 1H), 4.53-4.51 (m, 2H), 4.35-4.33 (m, 1H), 4.17 (s, 1H), 3.95-3.93 (m, 1H), 3.91 (s, 3H), 3.86-3.84 (m, 3H), 3.35 (s, 3H), 3.13-3.11 (m, 2H), 2.84-2.82 (m, 1H) 2.47 (s, 3H), 2.35 (s, 3H), 2.22-2.03 (m, 5H), 1.88-1.86 (m, 3H), 1.43-1.40 (m, 3H).

Compound 116 NMR: ¹H NMR (600 MHz, CDCl₃) δ 7.00 (s, 1H), 6.51 (s, 1H), 4.56-4.41 (m, 2H), 4.39-4.37 (m, 1H), 4.35-4.33 (m, 1H), 4.17-4.14 (m, 1H), 4.07 (s, 3H), 3.84-3.82 (m, 1H), 3.37 (s, 3H), 3.16-3.14 (m, 2H), 2.67 (s, 3H), 2.54-2.52 (m, 3H), 2.23 (s, 3H), 2.19-2.18 (m, 2H), 1.88 (s, 3H), 1.87-1.61 (m, 6H).

### Example 68, Compound 117 and 118 Synthesis

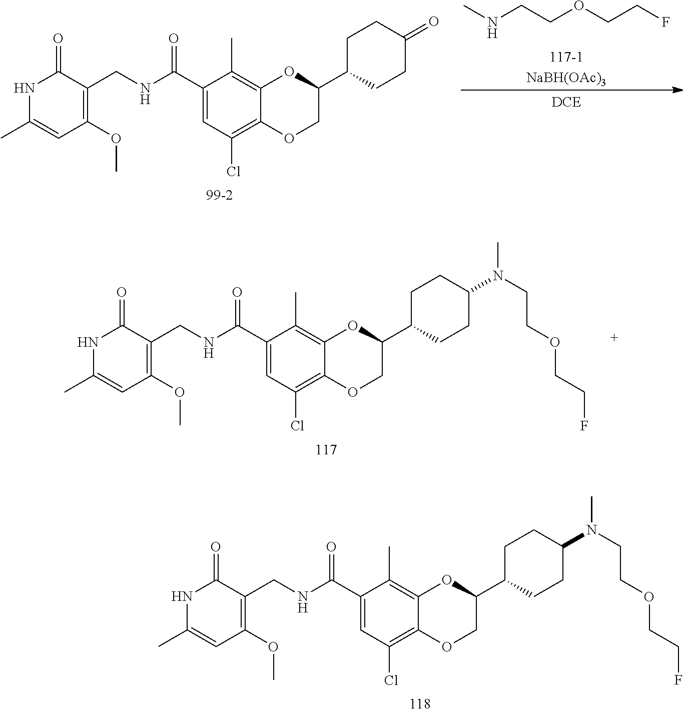

### Step 1, Compound 117 and 118 Synthesis

Following the synthesis method of Example 6, Step 4, except replacing Step 4, 6-3 with 99-2 (30.0 mg, 0.06 mmol), dimethylamine with 117-1 (15.0 mg, 0.1 mmol), the same synthesis method was applied. The obtained compound was subjected to Prep-MPLC to give Compound 117 (3.0 mg, 5.0 mol, 8.2% yield) and 118 (3.0 mg, 5.0 mol, 8.2% yield), LCMS (ESI⁺) m/z: 580.4 [M+H]⁺.

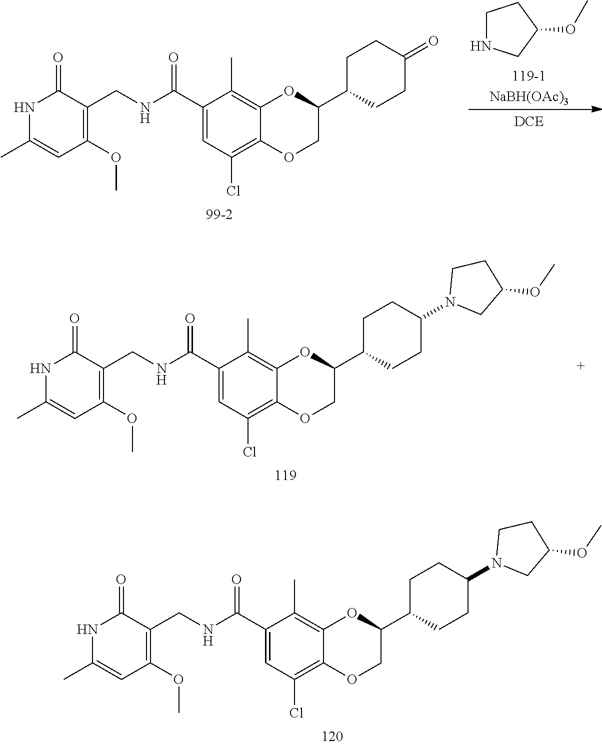

### Example 69, Compound 119 and 120 Synthesis

### Step 1, Compound 119 and 120 Synthesis

Following the synthesis method of Example 6, Step 4, except replacing Step 4, 6-3 with 99-2 (30.0 mg, 0.06 mmol), dimethylamine with 119-1 (7.7 mg, 7.6 μmol), the same synthesis method was applied. The obtained compound was subjected to Prep-MPLC to give Compound 119 (1.5 mg, 2.7 μmol, 4.2% yield) and 120 (3.5 mg, 6.3 μmol, 9.9% yield), LCMS (ESI⁺) m/z: 280.8

Compound 119 NMR: ¹H NMR (400 MHz, DMSO-d₆) δ 11.42 (s, 1H), 7.93 (t, J=4.8 Hz, 1H), 6.87 (s, 1H), 6.09 (s, 1H), 4.47-4.44 (m, 1H), 4.18 (d, J=4.4 Hz, 2H), 4.07-4.02 (m, 1H), 3.92-3.82 (m, 1H), 3.82-3.80 (m, 1H), 3.79 (s, 3H), 3.15 (s, 3H), 2.74-2.72 (m, 1H), 2.18 (s, 3H), 2.12 (s, 3H), 1.98-1.97 (m, 6H), 1.76-1.73 (m, 1H), 1.60-1.57 (m, 3H), 1.22-1.07 (m, 5H).

Compound 120 NMR: ¹H NMR (600 MHz, Methanol-d₄) δ 11.42 (s, 1H), 7.93 (t, J=4.8 Hz, 1H), 6.87 (s, 1H), 6.09 (s, 1H), 4.49 (s, 2H), 4.46 (dd, J=11.2, 2.0 Hz, 1H), 4.18 (d, J=4.8 Hz, 1H), 4.10-4.05 (m, 1H), 4.00-3.98 (m, 1H), 3.88-3.86 (m, 1H), 3.79 (s, 3H), 3.17 (s, 3H), 2.74-2.72 (m, 1H), 2.44-2.43 (m, 3H), 2.18 (s, 3H), 2.12 (s, 3H), 1.98-1.91 (m, 2H), 1.92-1.90 (m, 2H), 1.62-1.58 (i, 3H), 1.54-1.52 (m, 1H), 1.44-1.42 (m, 3H).

### Example 70, Compound 121, 122 and 123 Synthesis

### Step 1, Compound 121 and 122 Synthesis

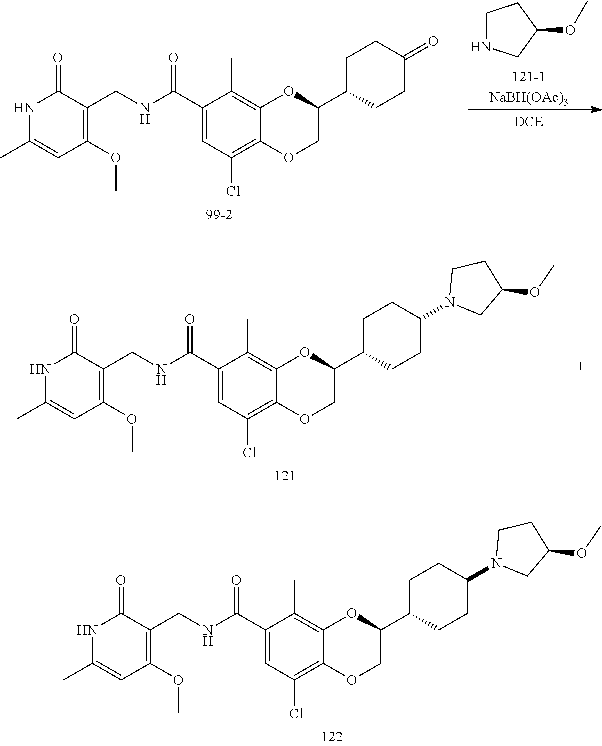

Following the synthesis method of Example 6, Step 4, except replacing Step 4, 6-3 with 99-2 (30.0 mg, 0.06 mmol), dimethylamine with 121-1 (6.3 mg, 6.3 mol), the same synthesis method was applied. The obtained compound was subjected to Prep-MPLC to give Compound 121 (2.5 mg, 4.5 mol, 7.1% yield) and 122 (2.4 mg, 4.3 mol, 6.8% yield), LCMS (ESI⁺) m/z: 281.0 [(M+H)/2+H]⁺.

Compound 121 NMR: ¹H NMR (400 MHz, Methanol-d₄) δ 6.91 (s, 1H), 6.25 (s, 1H), 4.86-4.39 (m, 3H), 4.08-4.03 (m, 1H), 3.96-3.94 (m, 1H), 3.92 (s, 3H), 3.90-3.86 (m, 1H), 3.31 (s, 3H), 2.90-2.86 (m, 4H), 2.80-2.66 (m, 1H), 2.31 (s, 3H), 2.18 (s, 3H), 2.12-2.04 (m, 4H), 1.89-1.84 (m, 2H), 1.66-1.64 (m, 1H), 1.33-1.26 (m, 4H).

Compound 122 NMR: ¹H NMR (400 MHz, Methanol-d₄) δ 6.93 (s, 1H), 6.25 (s, 1H), 4.49-4.46 (m, 1H), 4.44 (s, 2H), 4.21-4.15 (m, 1H), 4.06-4.02 (m, 2H), 3.92 (s, 3H), 3.31 (s, 3H), 3.06-3.04 (m, 3H), 2.92-2.88 (m, 1H), 2.65-2.61 (s, 1H), 2.31 (s, 3H), 2.19 (s, 3H), 2.15-2.12 (m, 1H), 2.03-1.99 (m, 2H), 1.88-1.59 (m, 8H).

### Compound 123 Synthesis

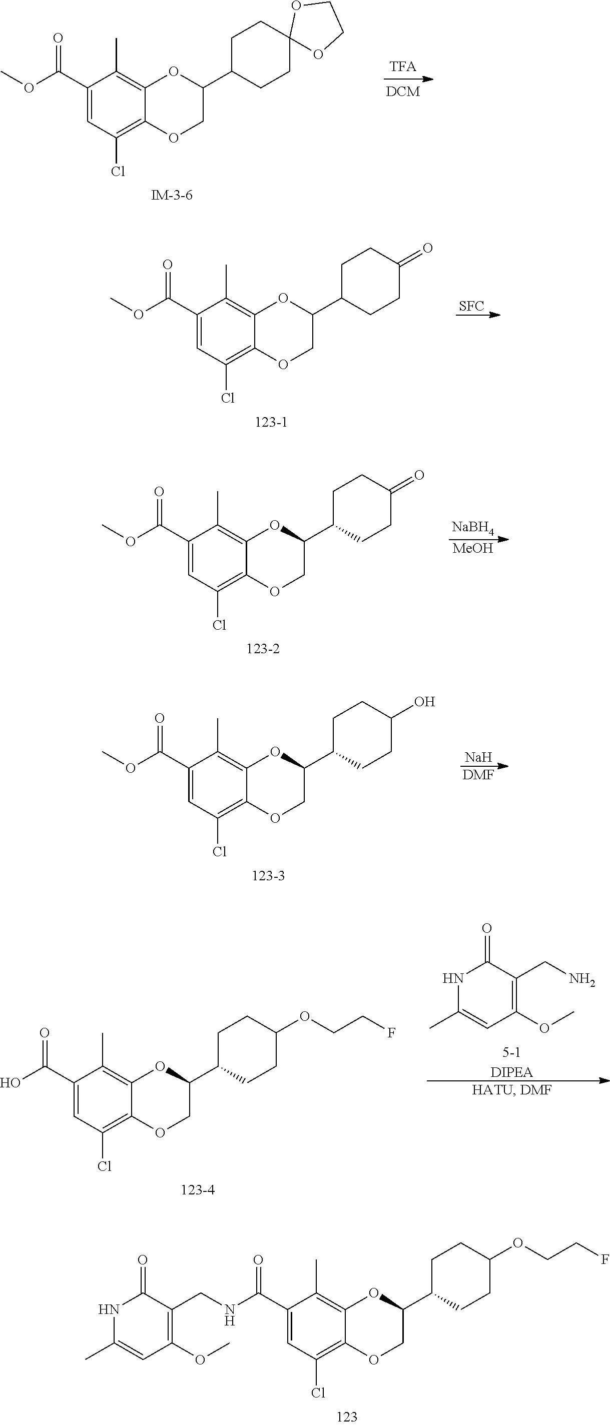

### Step 1, Compound 123-1 Synthesis

Following the synthesis method of Example 3, Step 2, except replacing Step 2, 3-2 with IM-3-6 (100.0 mg, 0.3 mmol), the same synthesis method was applied, to give Compound 123-1 (120.0 mg, crude), LCMS (ESI⁺) m/z: 338.8 [M+H]⁺.

### Step 2, Compound 123-2 Synthesis

Compound 123-2 can be obtained by SFC resolution from Compound 123-1.

### Step 3, Compound 123-3 Synthesis

Following the synthesis method of Example IM-3, Step 3, except replacing Step 3, IM-3-4 with 123-2 (40.0 mg, 0.1 mmol), the same synthesis method was applied, to give Compound 123-3 (36.2 mg, 0.1 mmol, 90.0% yield), LCMS (ESI⁺) m/z: 340.8 [M+H]⁺.

### Step 4, Compound 123-4 Synthesis

A single-necked flask was added with substrate 123-3 (8.0 mg, 0.02 mmol). The mixture was dissolved with DMF (1 mL) under stirring, and then at 0° C. and under nitrogen protection, added with NaH (9.4 mg, 0.2 mmol), and stirred to react for 0.5 hours, and then added with 1-fluoro-2-iodo-ethane (20.0 mg, 0.1 mmol), reacted at room temperature for 12 hours, and then added with water (5 mL) further stirred for 12 hours, with LC-MS monitoring. After the completion of the reaction, the reaction mixture was adjusted with 4 N HCl solution to pH=6, added with petroleum ether and ethyl acetate (8:1) to extract three times. The organic phase was combined, washed with saturated saline solution, and dried with anhydrous sodium sulfate and then condensated under reduced pressure to give the crude product 123-4 (8.0 mg, crude). LCMS (ESI⁺) m/z: 372.8 [M+H]⁺

### Step 4, Compound 123 Synthesis

Following the synthesis method of Example 3, Step 1, except replacing Step 1, 2-3 with 123-4 (8.0 mg, 0.02 mmol), 2-4 with 5-1 (3.6 mg, 0.02 mmol) the same synthesis method was applied, to give Compound 123 (1.2 mg, 2.3 mol, 9.8% yield), LCMS (ESI⁺) m/z: 523.4 [M+H]⁺. 1H NMR (400 MHz, CDCl₃) δ 10.85 (s, 1H), 7.14-7.11 (m, 1H), 6.93 (s, 1H), 5.89 (s, 1H), 4.55-4.46 (m, 1H), 4.44-4.42 (m, 2H), 4.42-4.41 (m, 1H), 4.32-4.30 (m, 1H), 3.94-3.92 (m, 1H), 3.83 (s, 3H), 3.71-3.70 (m, 1H), 3.69-3.63 (m, 1H), 3.62-3.60 (m, 1H), 3.28-3.21 (m, 1H), 2.25 (s, 3H), 2.18 (s, 3H), 2.09-2.06 (m, 3H), 1.75-1.72 (m, 1H), 1.18-1.15 (m, 2H), 0.80-0.77 (m, 3H).

### Example 71, Compound 124 Synthesis

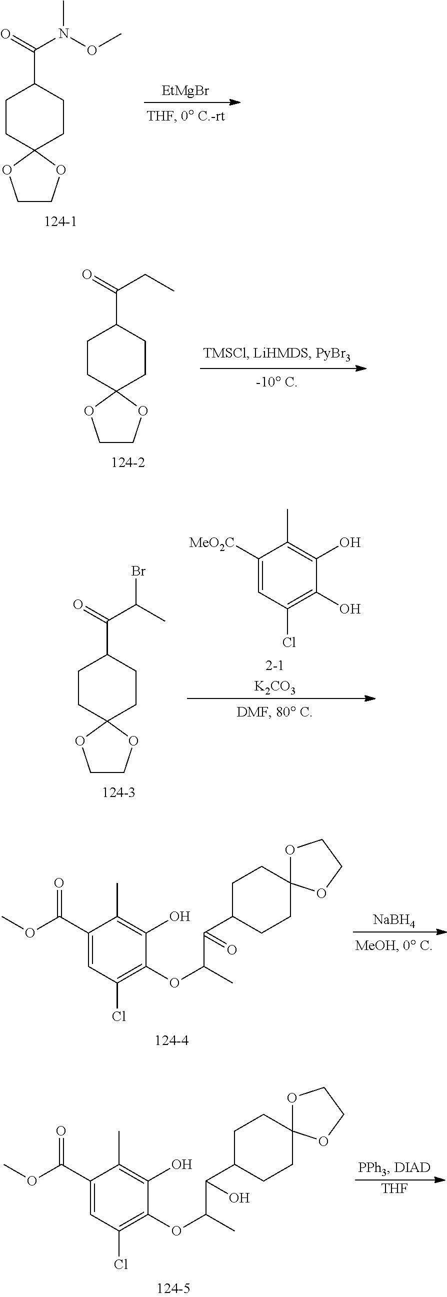

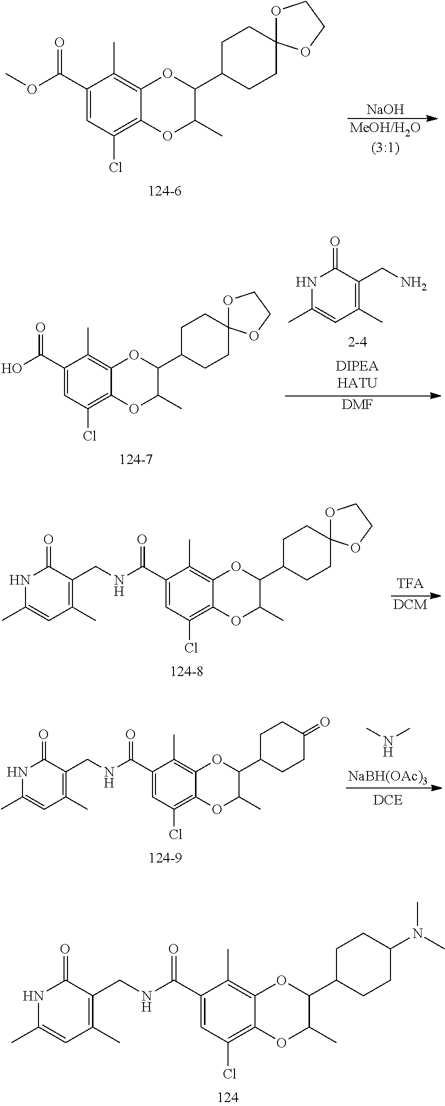

### Step 1, Compound 124-2 Synthesis

A single-necked flask was added with substrate 124-1 (1.0 g, 4.4 mmol). The mixture was dissolved with THF (20 mL) under stirring, and then at 0° C. added with EtMgBr (754.0 mg, 5.7 mmol), stirred to react for 16 h, with TLC monitoring. After the completion of the reaction, the mixture was added with saturated ammonium chloride solution and ethyl acetate to extract three times. The organic phase was combined, washed with saturated saline solution, and dried with anhydrous sodium sulfate, and then purified by silica gel column chromatography (PE:EA=5:1), to give the product 124-2 (500 mg, 2.5 mmol, 57.8% yield).

### Step 2, Compound 124-3 Synthesis

A single-necked flask was added with substrate 124-2 (500.0 mg, 2.5 mmol). The mixture was dissolved with THF (20 mL) under stirring, and then at 0° C. added with LiHMDS (842.0 mg, 5.0 mmol) stirred for 0.5 h, and then added with PyHBr₃ (1.2 g, 3.8 mmol), further stirred to react for 2 hours, with TLC monitoring. After the completion of the reaction, the mixture was added with saturated sodium bicarbonate solution and ethyl acetate to extract three times. The organic phase was combined, washed with saturated saline solution, and dried with anhydrous sodium sulfate, and then purified by silica gel column chromatography (PE:EA=5:1), to give the product 124-3 (240.0 mg, 0.9 mmol, 34.3% yield).

### Step 3, Compound 124-4 Synthesis

Following the synthesis method of Example 2, Step 1, except replacing Step 1, IM-2 with 123-3 (240.0 mg, 0.9 mmol), the same synthesis method was applied, to give Compound 124-4 (220.0 mg, 0.5 mmol, 61.5% yield).

### Step 4, Compound 124-5 Synthesis

Following the synthesis method of Example IM-3, Step 3, except replacing Step 3, IM-3-4 with 123-4 (220.0 mg, 0.5 mmol), the same synthesis method was applied, to give Compound 124-5 (180.0 mg, 0.4 mmol, 81.4% yield).

### Step 5, Compound 124-6 Synthesis

Following the synthesis method of Example IM-3, Step 4, except replacing Step 4, IM-3-5 with 123-5 (180.0 mg, 0.4 mmol), the same synthesis method was applied, to give Compound 124-6 (130.0 mg, 0.3 mmol, 75.5% yield).

### Step 6, Compound 124-7 Synthesis

A single-necked flask was added with substrate 124-6 (130.0 mg, 0.3 mmol). The mixture was dissolved with MeOH/H₂O (20 mL) to dissolve, and then added with NaOH (131.0 mg, 3.0 mmol), 80° C. to react for 1 hour, with LC-MS monitoring. After the completion of the reaction, the mixture was added with 1 N HCl to adjust to pH=6, and then added with EA solution to extract three times. The organic phase was combined, washed with saturated saline solution, and dried with anhydrous sodium sulfate and then condensated under reduced pressure, to give the crude product 124-7 (130.0 mg, crude). LCMS (ESI⁺) m/z: 382.8 [M+H]⁺

### Step 7, Compound 124-8 Synthesis

Following the synthesis method of Example 1, Step 3, except replacing Step 3, 1-3 with 124-7 (130.0 mg, crude), the same synthesis method was applied, to give Compound 124-8 (150.0 mg, crude), LCMS (ESI⁺) m/z: 517.0 [M+H]⁺.

### Step 8, Compound 124-9 Synthesis

Following the synthesis method of Example 3, Step 2, except replacing Step 2, 3-2 with 124-8 (150.0 mg, crude), the same synthesis method was applied, to give Compound 124-9 (120.0 mg, crude), LCMS (ESI⁺) m/z: 472.9 [M+H]⁺.

### Step 9, Compound 124 Synthesis

Following the synthesis method of Example 6, Step 4, except replacing Step 4, 6-3 with 124-9 (80.0 mg, 0.2 mmol), the same synthesis method was applied, to give Compound 124 (21.0 mg, 0.04 mmol, 24.7% yield), LCMS (ESI⁺) m/z: 502.6 [M+H]⁺.

¹H NMR (600 MHz, CDCl₃) δ 10.11 (s, 1H), 7.10-7.08 (m, 1H), 6.98 (s, 1H), 5.93 (s, 1H), 4.64-4.49 (m, 3H), 4.07-4.05 (m, 1H), 2.99-2.97 (m, 1H), 2.72 (s, 6H), 2.68-2.65 (m, 2H), 2.38 (s, 3H), 2.24 (s, 3H), 2.21 (s, 3H), 1.98-1.95 (m, 3H), 1.87-1.85 (m, 2H), 1.70-1.65 (m, 5H).

### Example 72, Compound 125 Synthesis

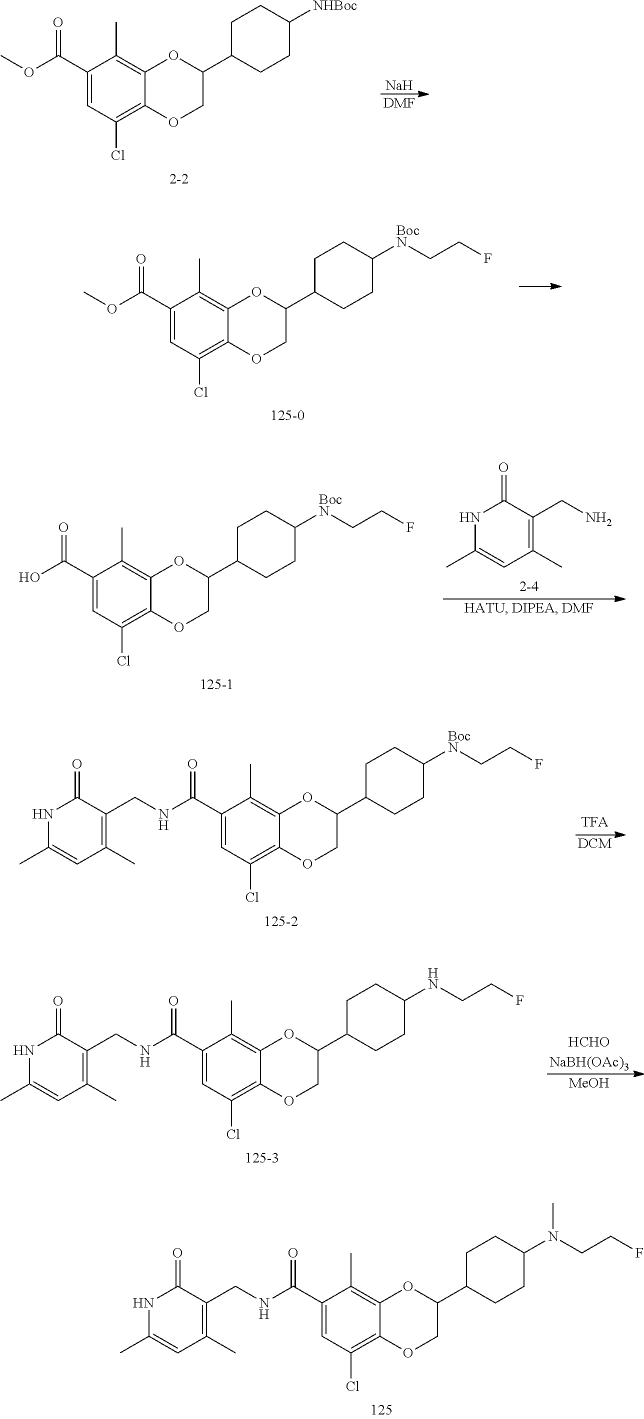

### Step 1, Compound 125-1 Synthesis

Following the synthesis method of Example 70, Step 4, except replacing Step 4, 123-3 with 2-2 (220.0 mg, 0.5 mmol), 1-fluoro-2-iodo-ethane with 1-bromo-2-fluoro-ethane (127.0 mg, 1.0 mmol), the same synthesis method was applied. After the completion of the reaction, the system was added with water, stirred at room temperature for 0.5 hours, with LC-MS monitoring. After the completion of the reaction, the mixture was added with 1 N HCl to adjust to pH=6, and then added with EA solution to extract three times. The organic phase was combined, washed with saturated saline solution, and dried with anhydrous sodium sulfate and then condensated under reduced pressure, to give Compound 125-1 (35.0 mg, 0.07 mmol, 14.8% yield), LCMS (ESI⁺) m/z: 471.9 [M+H]⁺.

### Step 2, Compound 125-2 Synthesis

Following the synthesis method of Example 1, Step 3, except replacing Step 3, 1-3 with 125-1 (35.0 mg, 0.07 mmol), the same synthesis method was applied, to give Compound 125-2 (20.0 mg, crude), LCMS (ESI⁺) m/z: 606.1 [M+H]⁺.

### Step 3, Compound 125-3 Synthesis

Following the synthesis method of Example 3, Step 2, except replacing Step 2, 3-2 with 125-2 (20.0 mg, crude), the same synthesis method was applied, to give Compound 125-3 (16.5 mg, crude), LCMS (ESI⁺) m/z: 506.1 [M+H]⁺.

### Step 4, Compound 125 Synthesis

Following the synthesis method of Example 1, Step 5, except replacing Step 5, 1-6 with 125-3 (16.5 mg, crude), the same synthesis method was applied, to give Compound 125 (8.0 mg, 15.4 mol, 47.1% yield), LCMS (ESI⁺) m/z: 520.2 [M+H]⁺.

¹H NMR (600 MHz, Methanol-d₄) δ 6.92 (s, 1H), 6.10 (s, 1H), 4.62-4.56 (m, 3H), 4.51 (t, J=4.8 Hz, 1H), 4.42 (m, 2H), 4.08-4.03 (m, 1H), 3.91-3.83 (m, 1H), 2.92-2.88 (m, 1H), 2.88-2.84 (m, 1H), 2.61-2.55 (s, 1H), 2.38 (s, 3H), 2.35 (s, 3H), 2.24 (s, 3H), 2.98 (s, 3H), 2.01-1.96 (m, 2H), 1.91-1.87 (m, 1H), 0.93-0.84 (m, 5H).

### Example 73, Compound 126 Synthesis

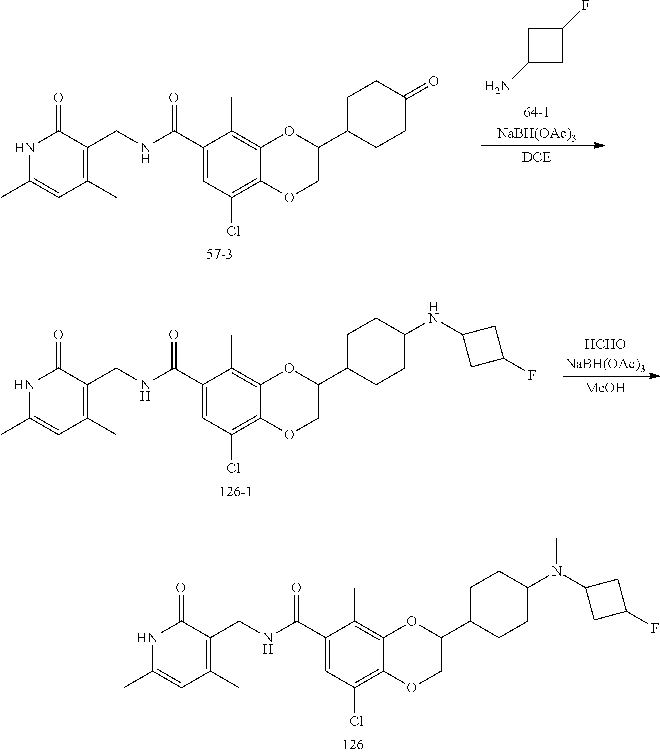

### Step 1, Compound 126-1 Synthesis

Following the synthesis method of Example 6, Step 4, except replacing Step 4, 6-3 with 57-3 (46.0 mg, 0.1 mmol), dimethylamine with 64-1 (13.0 mg, 0.2 mmol), the same synthesis method was applied, to give Compound 126-1 (167.0 mg, crude), LCMS (ESI⁺) m/z: 532.3 [M+H]⁺.

### Step 2, Compound 126 Synthesis

Following the synthesis method of Example 1, Step 5, except replacing Step 5, 1-6 with 126-1 (167.0 mg, crude), the same synthesis method was applied, to give Compound 126 (18.0 mg, 0.03 mmol, 10.5% yield), LCMS (ESI⁺) m/z: 546.3 [M+H]⁺.

¹H NMR (600 MHz, DMSO-d₆) δ 11.47 (s, 1H), 8.12 (s, 1H), 6.89 (s, 1H), 5.85 (s, 1H), 5.15-5.05 (m, 1H), 4.51-4.33 (m, 2H), 4.22 (s, 2H), 4.09-3.85 (m, 2H), 3.58-3.56 (m, 1H), 2.23-2.09 (m, 12H), 2.04-1.96 (m, 2H), 1.83-1.65 (m, 3H), 1.62-1.38 (m, 5H), 1.32-1.13 (m, 3H).

### Example 74, Compound 127 Synthesis

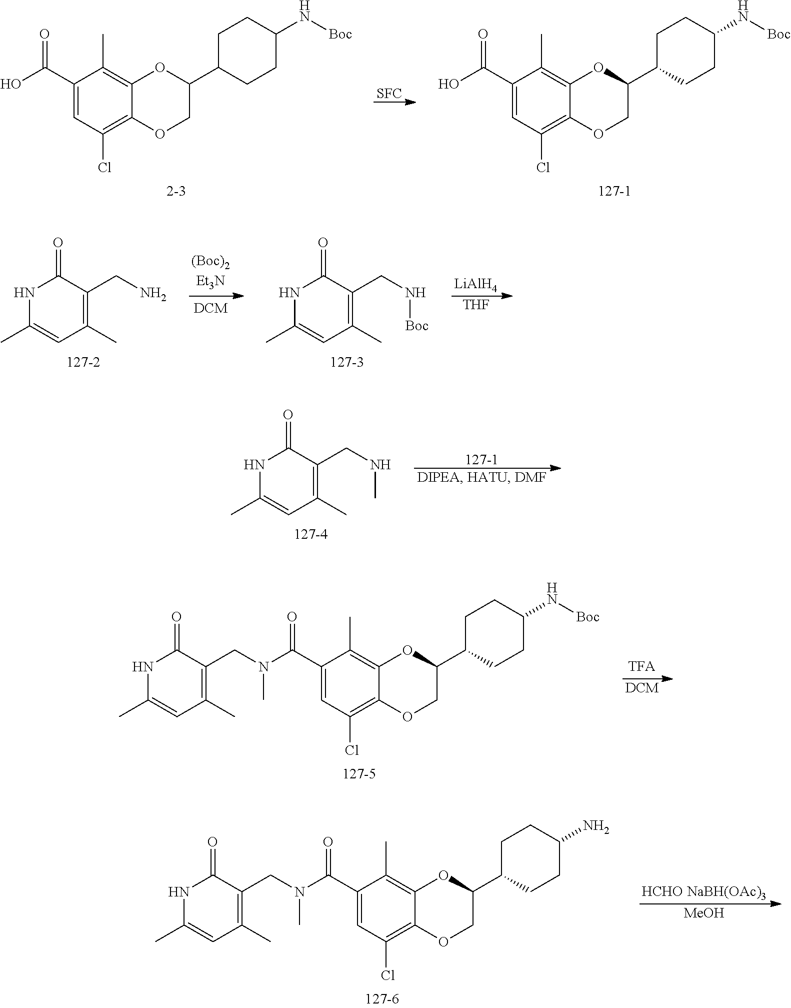

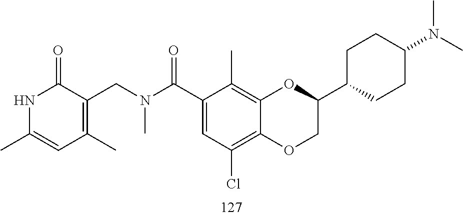

### Step 1, Compound 127-1 Synthesis

Compound 127-1 can be obtained by SFC resolution from Compound 2-3.

### Step 2, Compound 127-3 Synthesis

A single-necked flask was added with substrate 127-2 (636.7 mg, 2.9 mmol). The mixture was dissolved with DCM (5 mL) to dissolve, and then added with Et₃N (883.0 mg, 7.3 mmol), and reacted at room temperature for 1 hour, with LC-MS monitoring. After the completion of the reaction, and condensated under reduced pressure, and then the reaction mixture was purified by medium pressure liquid chromatography to give the product 127-3 (500.0 mg, 2.0 mmol, 47.4% yield). LCMS (ESI⁺) m/z: 252.3 [M+H]⁺

### Step 3, Compound 127-4 Synthesis

A single-necked flask was added with substrate 127-3 (500.0 mg, 2.0 mmol). The mixture was dissolved with THF (20 mL) to dissolve. Then at 0° C. and under nitrogen protection, the mixture was added with LiAlH₄ (75.2 mg, 2.0 mmol), stirred at 0° C. to react for 2 hours, with LC-MS monitoring. After the completion of the reaction, the reaction mixture was extracted with water and EA solution three times. The organic phase was combined, washed with saturated saline solution, and dried with anhydrous sodium sulfate and then condensated under reduced pressure, to give the crude product 127-4 (500.0 mg, crude). LCMS (ESI⁺) m/z: 166.2 [M+H]⁺

### Step 4, Compound 127-5 Synthesis

Following the synthesis method of Example 1, Step 3, except replacing Step 3, 1-3 with 127-1 (190.0 mg, 0.5 mmol), 1-4 with 127-4 (148.0 mg, 0.9 mmol), the same synthesis method was applied, to give Compound 127-5 (110.0 mg, 0.2 mmol, 43.0% yield), LCMS (ESI⁺) m/z: 574.1 [M+H]⁺.

### Step 5, Compound 127-6 Synthesis

Following the synthesis method of Example 3, Step 2, except replacing Step 2, 3-2 with 127-5 (110.0 mg, 0.2 mmol), the same synthesis method was applied, to give Compound 127-6 (98.0 mg, crude), LCMS (ESI⁺) m/z: 474.0 [M+H]⁺.

### Step 6, Compound 127 Synthesis

Following the synthesis method of Example 6, Step 4, except replacing Step 4, 6-3 with 127-6 (47.0 mg, 0.1 mmol), added with formaldehydewater solution (30.0 mg, 1.0 mmol), DCE with MeOH, the same synthesis method was applied, to give Compound 127 (35.0 mg, 0.07 mmol, 69.7% yield). LCMS (ESI⁺) m/z: 502.3 [M+H]⁺

¹H NMR (600 MHz, DMSO-d₆) δ 11.53-11.48 (m, 1H), 7.00 (s, 0.3H), 6.75 (s, 0.7H), 5.90 (s, 0.7H), 5.83 (s, 0.3H), 4.56-4.54 (m, 1H), 4.47-4.46 (m, 1H), 4.07-4.06 (m, 1H), 3.95-3.93 (m, 1H), 2.73 (s, 1H), 2.58 (s, 2H), 2.18 (s, 9H), 2.13 (s, 3H), 2.11 (s, 1H), 2.09 (s, 2H), 1.97 (s, 3H), 1.87-1.85 (m, 2H), 1.80-1.79 (m, 1H), 1.57-1.55 (m, 1H), 1.20-1.16 (m, 4H).

### Example 75, Compound 128 Synthesis

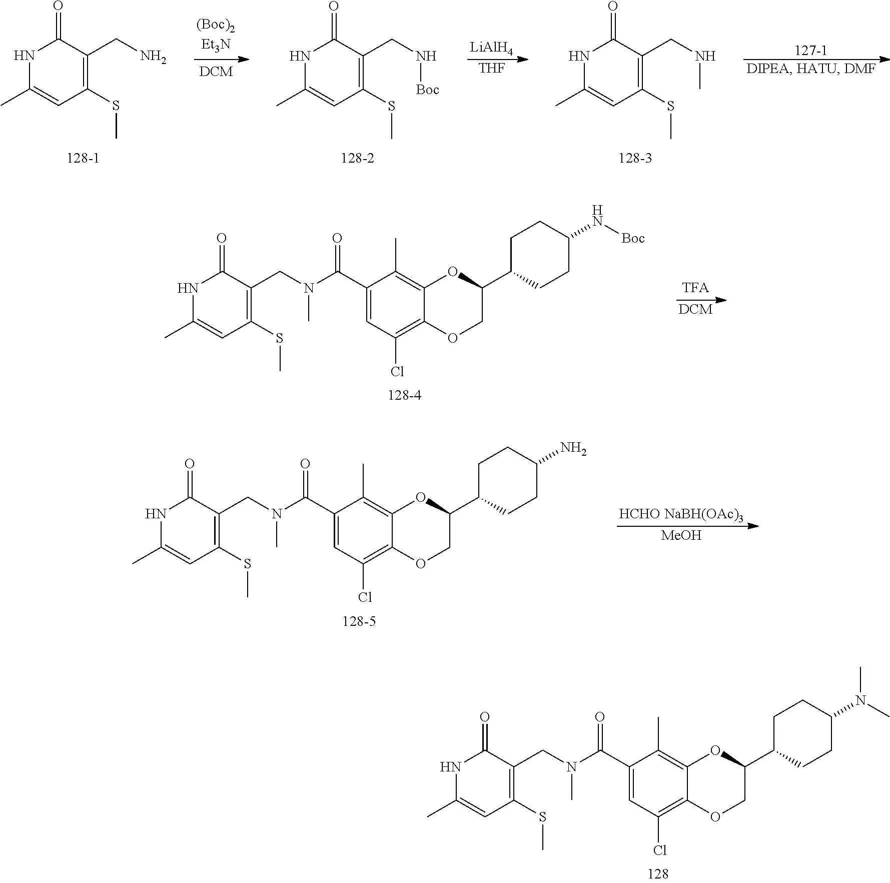

### Step 1, Compound 128-2 Synthesis

Following the synthesis method of Example 74, Step 2, except replacing Step 2, 127-2 with 128-1 (628.0 mg, 3.4 mmol), the same synthesis method was applied, to give Compound 128-2 (284.0 mg, crude), LCMS (ESI⁺) m/z: 284.4 [M+H]⁺.

### Step 2, Compound 128-3 Synthesis

Following the synthesis method of Example 74, Step 3, except replacing Step 3, 127-3 with 128-2 (284.0 mg, 1.0 mmol), the same synthesis method was applied, to give Compound 128-3 (100.0 mg, crude), LCMS (ESI⁺) m/z: 198.3 [M+H]⁺.

### Step 3, Compound 128-4 Synthesis

Following the synthesis method of Example 1, Step 3, except replacing Step 3, 1-3 with 127-1 (78.0 mg, 0.2 mmol), 1-4 with 128-3 (73.0 mg, 0.4 mmol), the same synthesis method was applied, to give Compound 128-4 (110.0 mg, crude), LCMS (ESI⁺) m/z: 606.2 [M+H]⁺.

### Step 4, Compound 128-5 Synthesis

Following the synthesis method of Example 3, Step 2, except replacing Step 2, 3-2 with 128-4 (30.0 mg, 0.05 mmol), the same synthesis method was applied, to give Compound 128-5 (50.0 mg, crude), LCMS (ESI⁺) m/z: 506.1 [M+H]⁺.

### Step 5, Compound 128 Synthesis

Following the synthesis method of Example 6, Step 4, except replacing Step 4, 6-3 with 128-5 (50.0 mg, crude), added with formaldehydewater solution (60.0 mg, 2.0 mmol), DCE with MeOH, the same synthesis method was applied, to give Compound 128 (10.0 mg, 18.7 mol, 18.9% yield).

LCMS (ESI⁺) m/z: 534.3 [M+H]⁺

¹H NMR (600 MHz, DMSO-d₆) δ 11.49 (m, 1H), 6.98-6.77 (m, 1H), 6.18-6.09 (m, 1H), 4.63-4.58 (m, 1H), 4.47-4.42 (m, 1H), 4.24-3.96 (m, 3H), 3.14-3.09 (m, 1H), 2.75-2.71 (m, 6H), 2.54-2.53 (m, 4H), 2.48-2.47 (m, 1H), 2.43 (s, 1H), 2.22 (s, 2H), 2.19 (m, 1H), 2.08-2.04 (m, 5H), 1.91-1.88 (m, 1H), 1.67-1.63 (m, 1H), 1.48-1.39 (m, 2H), 1.35-1.27 (m, 2H).

### Example 76, Compound 129 Synthesis

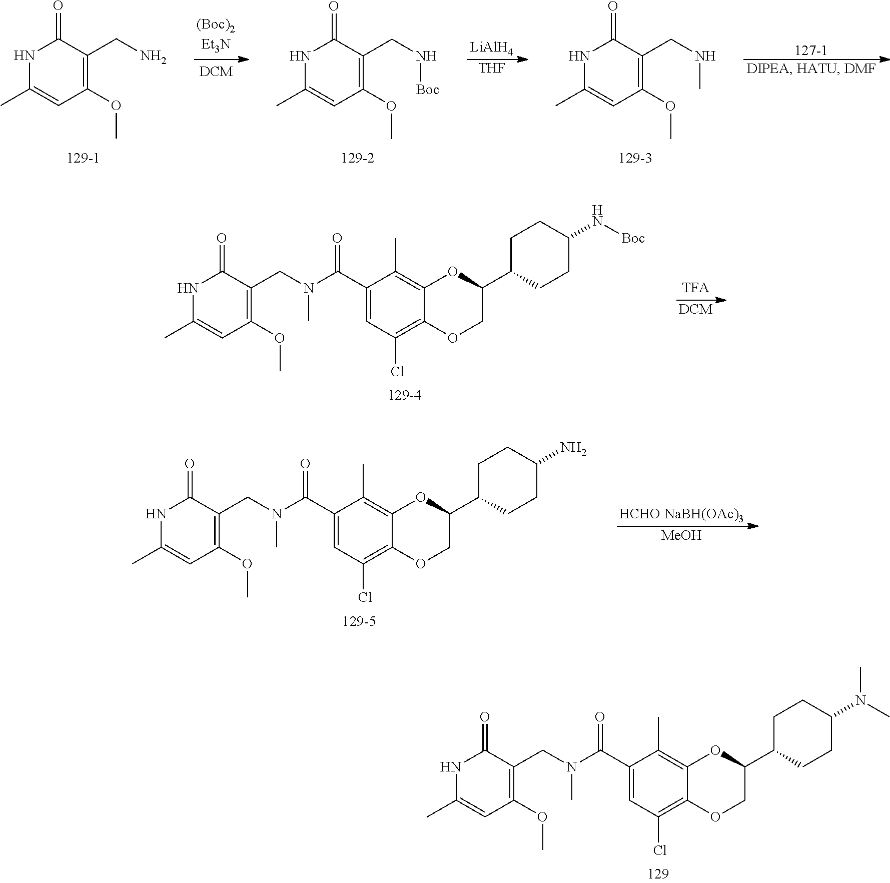

### Step 1, Compound 129-2 Synthesis

Following the synthesis method of Example 74, Step 2, except replacing Step 2, 127-2 with 129-1 (422.0 mg, 2.5 mmol), the same synthesis method was applied, to give Compound 129-2 (282.0 mg, crude), LCMS (ESI⁺) m/z: 268.3 [M+H]⁺.

### Step 2, Compound 129-3 Synthesis

Following the synthesis method of Example 74, Step 3, except replacing Step 3, 127-3 with 129-2 (282.0 mg, 1.1 mmol), the same synthesis method was applied, to give Compound 129-3 (100.0 mg, crude), LCMS (ESI⁺) m/z: 182.2 [M+H]⁺.

### Step 3, Compound 129-4 Synthesis

Following the synthesis method of Example 1, Step 3, except replacing Step 3, 1-3 with 127-1 (37.0 mg, 0.09 mmol), 1-4 with 129-3 (48.0 mg, 0.3 mmol), the same synthesis method was applied, to give Compound 129-4 (100.0 mg, crude), LCMS (ESI⁺) m/z: 590.1 [M+H]⁺.

### Step 4, Compound 129-5 Synthesis

Following the synthesis method of Example 3, Step 2, except replacing Step 2, 3-2 with 129-4 (60.0 mg, 0.1 mmol), the same synthesis method was applied, to give Compound 129-5 (50.0 mg, crude), LCMS (ESI⁺) m/z: 490.0 [M+H]⁺.

### Step 5, Compound 129 Synthesis

Following the synthesis method of Example 6, Step 4, except replacing Step 4, 6-3 with 129-5 (50.0 mg, crude), added with formaldehydewater solution (60.0 mg, 2.0 mmol), DCE with MeOH, the same synthesis method was applied, to give Compound 129 (12.0 mg, 23.2 mol, 22.7% yield).

LCMS (ESI⁺) m/z: 518.3 [M+H]⁺

¹H NMR (600 MHz, DMSO-d₆) δ 11.45 (s, 1H), 6.89-6.73 (m, 1H), 6.12-6.04 (m, 1H), 4.50-4.45 (m, 1H), 4.14-3.94 (m, 4H), 3.80 (s, 1H), 3.70 (s, 2H), 2.78 (s, 2H), 2.47 (s, 1H), 2.23-2.16 (m, 11H), 2.02-1.98 (m, 4H), 1.87-1.81 (m, 3H), 1.21-1.18 (m, 4H).

### Example 77, Compound 130 Synthesis

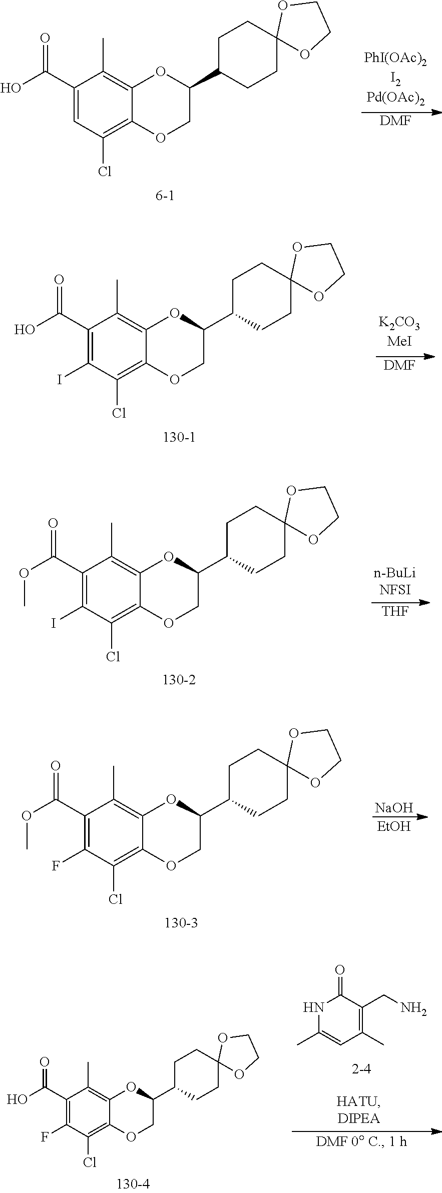

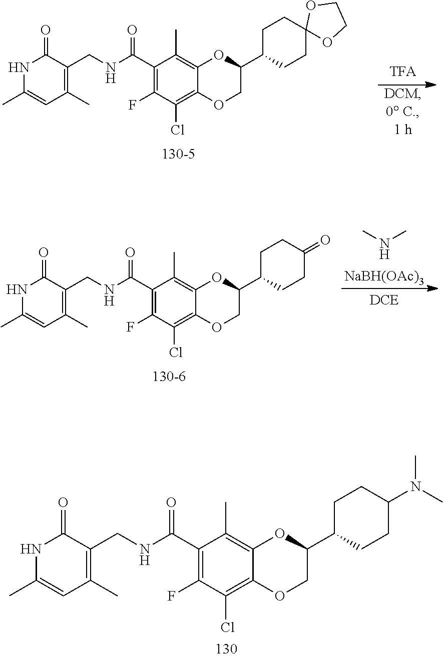

### Step 1, Compound 130-1 Synthesis

A single-necked flask was added with substrate 6-1 (520.0 mg, 1.4 mmol). The mixture was dissolved with DMF (5 mL) to dissolve, and then added with PhI(OAc)₂ (450.0 mg, 1.4 mmol), I₂ (358.0 mg, 1.4 mmol) and Pd(OAc)₂ (317.0 mg, 1.4 mmol), Under nitrogen protection10stirred at 0° C. to react for 24 hours, with LC-MS monitoring. After the completion of the reaction, the reaction mixture was cooled to room temperature, the reaction mixture was condensated under reduced pressure, and then extracted with water and EA solution three times. The organic phase was combined, washed with saturated saline solution, and dried with anhydrous sodium sulfate and then condensated under reduced pressure, to give the crude product 130-1 (480.0 mg, crude). LCMS (ESI⁺) m/z: 494.7 [M+H]⁺

### Step 2, Compound 130-2 Synthesis

A single-necked flask was added with substrate 130-1 (480.0 mg, crude). The mixture was dissolved with DMF (2 mL) to dissolve, and then added with K₂CO₃ (270.0 mg, 2.0 mmol) and Mel (275.0 mg, 2.0 mmol), stirred at 50° C. to react for 1 hour, with LC-MS monitoring. After the completion of the reaction, the reaction mixture was cooled to room temperature, the reaction mixture was condensated under reduced pressure, and then extracted with water and EA solution three times. The organic phase was combined, washed with saturated saline solution, and dried with anhydrous sodium sulfate and then condensated under reduced pressure, to give the crude product 130-2 (465.0 mg, crude). LCMS (ESI⁺) m/z: 508.7 [M+H]⁺

### Step 3, Compound 130-3 Synthesis

A single-necked flask was added with substrate 130-2 (152.0 mg, 0.3 mmol). The mixture was dissolved with THF (3 mL) to dissolve, and then under nitrogen protection and at −78° C. slowly added dropwise with n-BuLi (2.5 M, 240 μL), at −78° C. stirred to react for 5 mins, and then added with NFSI (189.0 mg, 0.6 mmol), with LC-MS monitoring. After the completion of the reaction, the reaction mixture was cooled to room temperature, the reaction mixture was condensated under reduced pressure, and then extracted with water and EA solution three times. The organic phase was combined, washed with saturated saline solution, and dried with anhydrous sodium sulfate and then condensated under reduced pressure, and then purified by medium pressure liquid chromatography to give the product 130-3 (46.0 mg, 0.1 mmol, 38.4% yield). LCMS (ESI⁺) m/z: 400.8 [M+H]⁺

### Step 4, Compound 130-4 Synthesis

Following the synthesis method of Example 6, Step 1, except replacing Step 1, IM-3a with 130-3 (45.0 mg, 0.1 mmol), the same synthesis method was applied, to give Compound 130-4 (45.0 mg, crude), LCMS (ESI⁺) m/z: 386.8 [M+H]⁺.

### Step 5, Compound 130-5 Synthesis

Following the synthesis method of Example 1, Step 3, except replacing Step 3, 1-3 with 130-4 (45.0 mg, crude), 1-4 with 2-4 (21.0 mg, 0.2 mmol), the same synthesis method was applied, to give Compound 130-5 (70.0 mg, crude), LCMS (ESI⁺) m/z: 521.0 [M+H]⁺.

### Step 6, Compound 130-6 Synthesis

Following the synthesis method of Example 3, Step 2, except replacing Step 2, 3-2 with 130-5 (70.0 mg, crude), the same synthesis method was applied, to give Compound 130-6 (65.0 mg, crude), LCMS (ESI⁺) m/z: 477.0 [M+H]⁺.

### Step 7, Compound 130 Synthesis

Following the synthesis method of Example 6, Step 4, except replacing Step 4, 6-3 with 130-6 (65.0 mg, crude), the same synthesis method was applied, to give Compound 130 (20.0 mg, 39.5 μmol, 29.0% yield), LCMS (ESI⁺) m/z: 506.1 [M+H]⁺. 1H NMR (400 MHz, DMSO-d₆) δ 11.46 (s, 1H), 8.40 (t, J=4.8 Hz, 1H), 5.85 (s, 1H), 4.49 (dd, J=11.2, 1.6 Hz, 1H), 4.26 (d, J=5.2 Hz, 2H), 4.12-4.00 (m, 2H), 2.17-2.14 (m, 9H), 2.11 (s, 3H), 2.02 (s, 3H), 1.85-1.15 (m, 10H).

¹⁹F NMR (376 MHz, DMSO-d₆) δ −128.42.

### Example 78, Compound 131 and 132 Synthesis

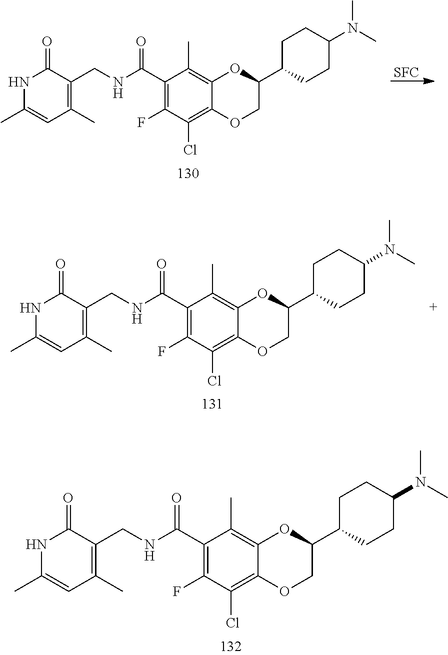

### Step 1, Compound 131 and 132 Synthesis

Compound 131 and 132 can be obtained by SFC resolution from Compound 130.

Compound 131 NMR: ¹H NMR (600 MHz, DMSO-d₆) δ 11.46 (s, 1H), 8.40 (t, J=4.8 Hz, 1H), 5.85 (s, 1H), 4.49-4.47 (m, 1H), 4.26 (d, J=4.8 Hz, 2H), 4.11-4.08 (m, 1H), 4.03-4.01 (m, 1H), 2.17-2.13 (m, 9H), 2.11 (s, 3H), 2.08 (s, 3H), 1.76-1.41 (m, 10H).

¹⁹F NMR (564 MHz, DMSO-d₆) δ −128.42.

Compound 132 NMR: ¹H NMR (600 MHz, DMSO-d₆) δ 11.47 (s, 1H), 8.39 (t, J=4.8 Hz, 1H), 5.85 (s, 1H), 4.48 (d, J=10.2 Hz, 1H), 4.26 (d, J=4.8 Hz, 2H), 4.10-4.06 (m, 1H), 3.89-3.86 (m, 1H), 2.16-2.15 (m, 9H), 2.10 (s, 3H), 2.03 (s, 3H), 1.88-1.77 (m, 3H), 1.30-1.23 (m, 4H), 1.19-1.11 (m, 3H).

¹⁹F NMR (564 MHz, DMSO-d₆) δ −128.42.

### Example 79, Compound 133 Synthesis

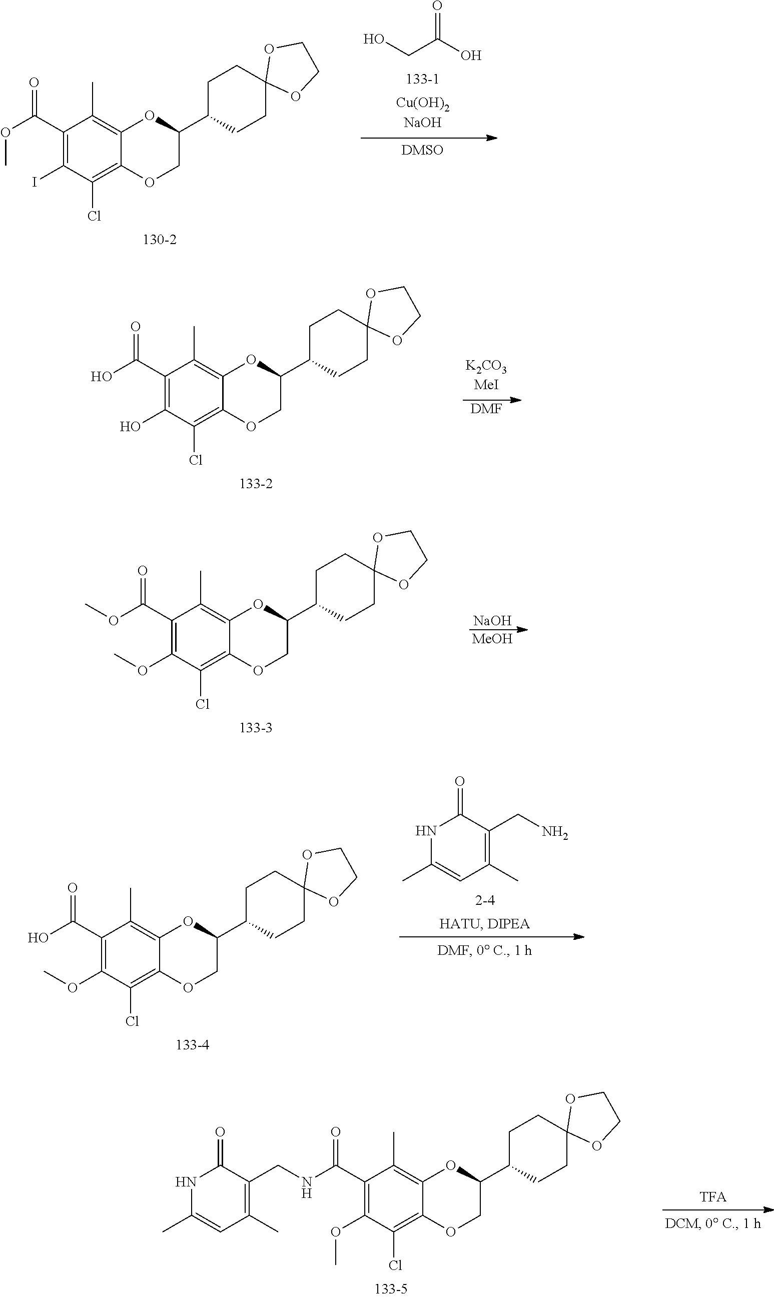

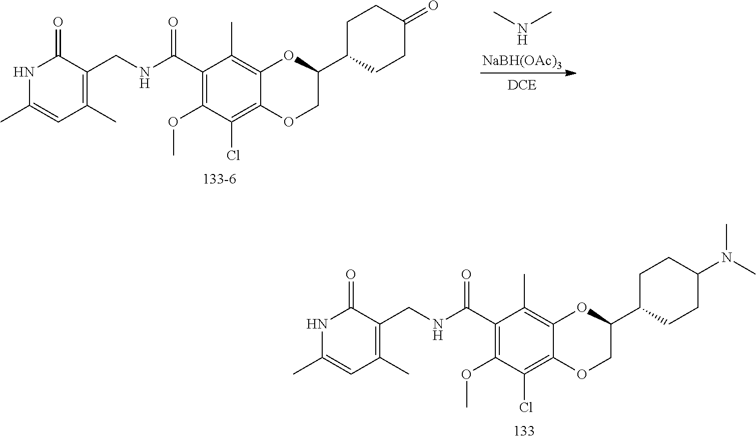

### Step 1, Compound 133-2 Synthesis

A single-necked flask was added with substrate 130-2 (110.0 mg, 0.2 mmol). The mixture was dissolved with DMSO (0.5 mL) and water (0.5 mL) to dissolve, and then added with 133-1 (5.0 mg, 65.8 mol), NaOH (52.0 mg, 1.3 mmol) and Cu(OH)₂ (1.0 mg, 10.0 mol), and at stirred at 120° C. to react for 6 hours, with LC-MS monitoring. After the completion of the reaction, the reaction mixture was cooled to room temperature, the reaction mixture was condensated under reduced pressure, and then extracted with water and EA solution three times. The organic phase was combined, washed with saturated saline solution, and dried with anhydrous sodium sulfate and then condensated under reduced pressure, and then purified by medium pressure liquid chromatography to give the product 133-2 (50.0 mg, crude). LCMS (ESI⁺) m/z: 384.8 [M+H]⁺

### Step 2, Compound 133-3 Synthesis

Following the synthesis method of Example 77, Step 2, except replacing Step 2, 130-1 with 133-2 (50.0 mg, crude), the same synthesis method was applied, to give Compound 133-3 (50.0 mg, crude), LCMS (ESI⁺) m/z: 412.9 [M+H]⁺.

### Step 3, Compound 133-4 Synthesis

Following the synthesis method of Example 77, Step 2, except replacing Step 2, 130-1 with 133-3 (50.0 mg, crude), the same synthesis method was applied, to give Compound 133-4 (50.0 mg, crude), LCMS (ESI⁺) m/z: 399.2 [M+H]⁺.

### Step 4, Compound 133-5 Synthesis

Following the synthesis method of Example 1, Step 3, except replacing Step 3, 1-3 with 133-4 (25.0 mg, 0.06 mmol), 1-4 with 2-4 (19.0 mg, 0.1 mmol), the same synthesis method was applied, to give Compound 133-5 (35.0 mg, crude), LCMS (ESI⁺) m/z: 533.0 [M+H]⁺.

### Step 5, Compound 133-6 Synthesis

Following the synthesis method of Example 3, Step 2, except replacing Step 2, 3-2 with 133-5 (35.0 mg, crude), the same synthesis method was applied, to give Compound 133-6 (30.0 mg, crude), LCMS (ESI⁺) m/z: 489.0 [M+H]⁺.

### Step 6, Compound 133 Synthesis

Following the synthesis method of Example 6, Step 4, except replacing Step 4, 6-3 with 133-6 (30.0 mg, crude), the same synthesis method was applied, to give Compound 133 (7.6 mg, 14.7 mol, 23.9% yield), LCMS (ESI⁺) m/z: 518.3 [M+H]⁺.

### Example 80, Compound 134 and 135 Synthesis

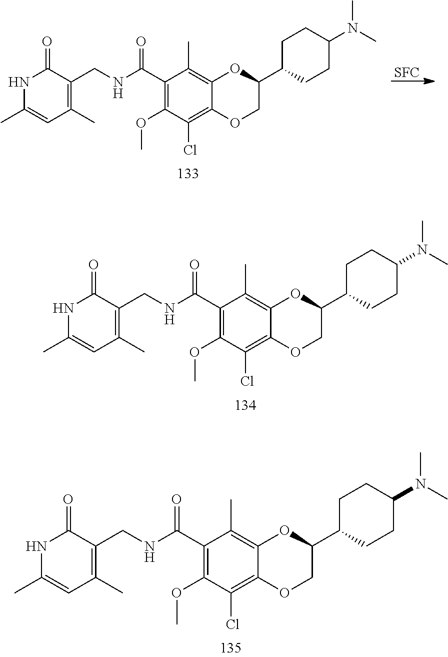

### Step 1, Compound 134 and 135 Synthesis

Compound 134 and 135 can be obtained by SFC resolution from Compound 133.

Compound 134 NMR: ¹H NMR (400 MHz, DMSO-d₆) δ 11.44 (s, 1H), 8.13 (t, J=5.2 Hz, 1H), 5.85 (s, 1H), 4.44 (dd, J=10.8, 1.6 Hz, 1H), 4.27 (d, J=5.2 Hz, 2H), 4.07-3.97 (m, 2H), 3.64 (s, 3H), 2.19 (s, 3H), 2.15 (s, 6H), 2.10 (s, 3H), 2.08-2.07 (m, 1H), 1.97 (s, 3H), 1.76-1.75 (m, 2H), 1.69-1.67 (m, 2H), 1.65-1.58 (m, 2H), 1.44-1.41 (m, 3H).

Compound 135 NMR: ¹H NMR (400 MHz, DMSO-d₆) δ 11.44 (s, 1H), 8.13 (t, J=4.8 Hz, 1H), 5.85 (s, 1H), 4.44 (dd, J=11.6, 2.0 Hz, 1H), 4.27 (d, J=4.8 Hz, 2H), 4.07-4.02 (m, 1H), 3.87-3.85 (m, 1H), 3.64 (s, 3H), 2.23 (s, 6H), 2.20 (s, 3H), 2.10 (s, 3H), 2.06-2.02 (m, 1H), 1.99 (s, 3H), 1.90-1.78 (m, 4H), 1.54-1.50 (m, 2H), 1.19-1.15 (m, 3H).

### Example 81, Compound 136 Synthesis

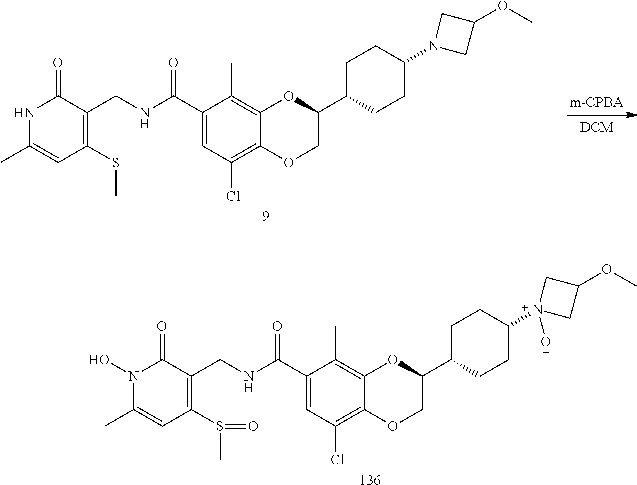

### Step 1, Compound 136 Synthesis

A single-necked flask was added with substrate 9 (20.0 mg, 0.04 mmol). The mixture was dissolved with DCM (5 mL) to dissolve, and then at 0° C. added with m-CPBA (18.0 mg, 0.1 mmol) stirred at 0° C. to react for 4 hours, with LC-MS monitoring. After the completion of the reaction, the reaction mixture was condensated under reduced pressure, and then purified by medium pressure liquid chromatography to give the product 136 (6.0 mg, 9.8 mol, 27.6% yield). LCMS (ESI⁺) m/z: 610.5 [M+H]⁺

¹H NMR (600 MHz, DMSO-d₆) δ 12.56 (s, 1H), 8.19 (t, J=4.8 Hz, 1H), 6.93 (s, 1H), 6.44 (s, 1H), 4.59 (d, J=3.6 Hz, 2H), 4.48-4.45 (m, 2H), 4.27-4.25 (m, 2H), 4.08-4.04 (m, 1H), 3.94-3.91 (m, 3H), 3.36 (s, 3H), 3.21 (s, 3H), 3.10-3.06 (m, 1H), 2.26 (s, 3H), 2.14 (s, 3H), 2.05-2.03 (m, 1H), 1.88-1.82 (m, 3H), 1.56-1.51 (m, 3H), 1.30-1.19 (m, 2H).

### Example 82, Compound 137 and 138 Synthesis

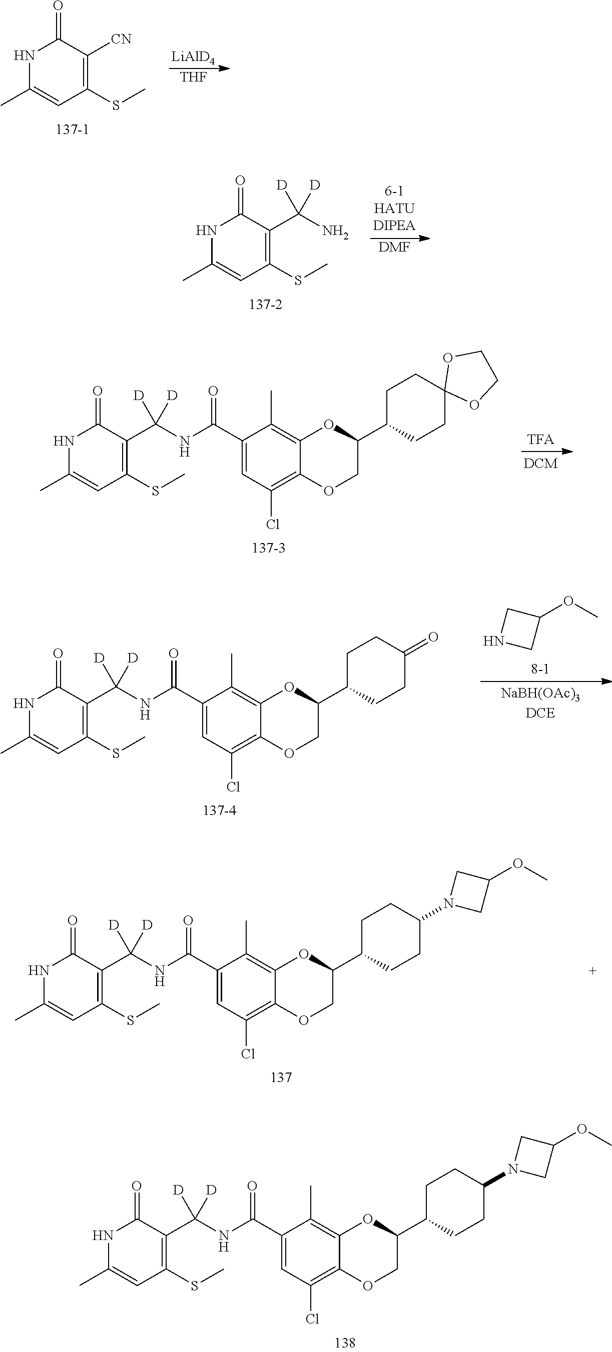

### Step 1, Compound 137-2 Synthesis

A single-necked flask was added with substrate 137-1 (36.0 mg, 0.2 mmol). The mixture was dissolved with THF (1 mL) to dissolve, and then at 0° C. added with LiAlD4 (8.4 mg, 0.2 mmol) stirred at 0° C. to react for 1 hour, with LC-MS monitoring. After the completion of the reaction, the reaction mixture was condensated under reduced pressure, and then extracted with water and EA solution three times. The organic phase was combined, washed with saturated saline solution, and dried with anhydrous sodium sulfate and then condensated under reduced pressure to give the crude product 137-2 (36.0 mg, crude). LCMS (ESI⁺) m/z: 187.2 [M+H]⁺

### Step 2, Compound 137-3 Synthesis

Following the synthesis method of Example 1, Step 3, except replacing Step 3, 1-3 with 6-1 (36.0 mg, 0.1 mmol), 1-4 with 137-2 (36.0 mg, 0.2 mmol), the same synthesis method was applied, to give Compound 137-3 (14.0 mg, 26.1 mol, 26.7% yield), LCMS (ESI⁺) m/z: 537.0 [M+H]⁺.

### Step 3, Compound 137-4 Synthesis

Following the synthesis method of Example 3, Step 2, except replacing Step 2, 3-2 with 137-3 (14.0 mg, 0.03 mmol), the same synthesis method was applied, to give Compound 137-4 (14.0 mg, crude), LCMS (ESI⁺) m/z: 493.0 [M+H]⁺.

### Step 4, Compound 137 and 138 Synthesis

Following the synthesis method of Example 6, Step 4, except replacing Step 4, 6-3 with 137-4 (14.0 mg, crude), dimethylamine with 8-1 (12.0 mg, 0.09 mmol) the same synthesis method was applied. The obtained compound was subjected to Prep-MPLC to give 137 (1.0 mg, 1.8 mol, 6.3% yield) and 138 (2.5 mg, 4.4 mol, 15.6% yield), LCMS (ESI⁺) m/z: 564.3 [M+H]⁺.

Compound 137 NMR: ¹H NMR (400 MHz, DMSO-d₆) δ 11.51 (s, 1H), 7.99 (s, 1H), 6.89 (s, 1H), 6.08 (s, 1H), 4.46-4.43 (m, 1H), 4.07-4.02 (m, 1H), 3.93-3.87 (m, 2H), 3.45-3.42 (m, 2H), 3.13 (s, 3H), 2.73-2.70 (m, 2H), 2.45 (s, 3H), 2.16 (s, 3H), 2.14 (s, 3H), 1.98-1.87 (m, 2H), 1.78-1.70 (m, 3H), 1.54-1.51 (m, 1H), 1.19-1.13 (m, 2H), 0.89-0.84 (m, 2H).

Compound 138 NMR: ¹H NMR (400 MHz, DMSO-d₆) δ 11.51 (s, 1H), 7.99 (s, 1H), 6.89 (s, 1H), 6.08 (s, 1H), 4.44-4.40 (m, 1H), 4.08-4.03 (m, 1H), 3.94-3.90 (m, 2H), 3.47-3.43 (m, 2H), 3.14 (s, 3H), 2.68-2.65 (m, 2H), 2.45 (s, 3H), 2.22-2.20 (m, 1H), 2.16 (s, 3H), 2.13 (s, 3H), 1.63-1.31 (m, 9H).

### Example 83, Compound 139 and 140 Synthesis

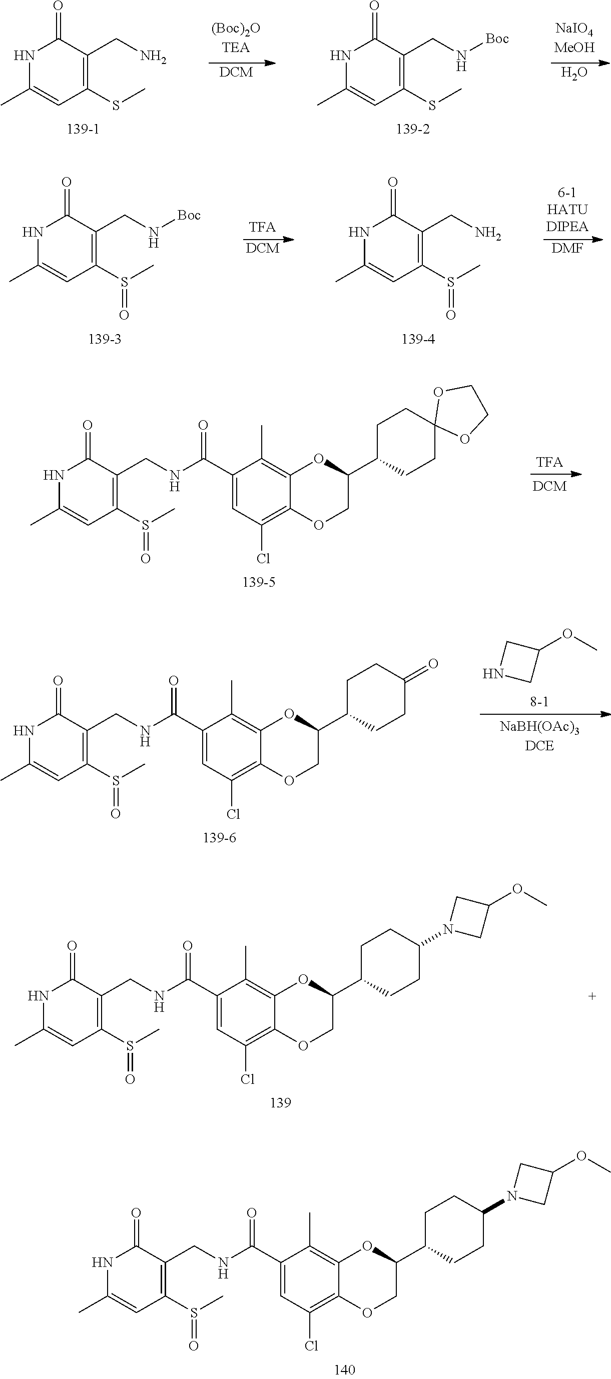

### Step 1, Compound 139-2 Synthesis

A single-necked flask was added with substrate 139-1 (184.0 mg, 1.0 mmol). The mixture was dissolved with DCM (3 mL) to dissolve, and then added with (Boc)₂O (327.0 mg, 1.5 mmol) and TEA (202.0 mg, 2.0 mmol, 279.0 L), at 25° C. stirred to react for 1 hour, with LC-MS monitoring. After the completion of the reaction, the reaction mixture was condensated under reduced pressure, and then extracted with water and EA solution three times. The organic phase was combined, washed with saturated saline solution, and dried with anhydrous sodium sulfate and then condensated under reduced pressure to give the crude product 139-2 (280.0 mg, crude). LCMS (ESI⁺) m/z: 284.4 [M+H]⁺

### Step 2, Compound 139-3 Synthesis

A single-necked flask was added with substrate 139-2 (100.0 mg, 0.4 mmol). The mixture was dissolved with MeOH (3 mL) and H₂O (1 mL) to dissolve, and then added with NaIO₄ (150.0 mg, 0.7 mmol), at 25° C. stirred to react for 36 hours, with LC-MS monitoring. After the completion of the reaction, the reaction mixture was condensated under reduced pressure, and then extracted with water and EA solution three times. The organic phase was combined, washed with saturated saline solution, and dried with anhydrous sodium sulfate and then condensated under reduced pressure to give the crude product 139-3 (63.0 mg, crude). LCMS (ESI⁺) m/z: 300.4 [M+H]⁺

### Step 3, Compound 139-4 Synthesis

Following the synthesis method of Example 3, Step 2, except replacing Step 2, 3-2 with 139-3 (63.0 mg, crude), the same synthesis method was applied, to give Compound 139-4 (40.0 mg, crude), LCMS (ESI⁺) m/z: 200.3 [M+H]⁺.

### Step 4, Compound 139-5 Synthesis

Following the synthesis method of Example 1, Step 3, except replacing Step 3, 1-3 with 6-1 (74.0 mg, 0.2 mmol), 1-4 with 139-4 (40.0 mg, crude), the same synthesis method was applied, to give Compound 139-5 (120.0 mg, crude), LCMS (ESI⁺) m/z: 551.1 [M+H]⁺.

### Step 5, Compound 139-6 Synthesis

Following the synthesis method of Example 3, Step 2, except replacing Step 2, 3-2 with 139-5 (120.0 mg, crude), the same synthesis method was applied, to give Compound 139-6 (120.0 mg, crude), LCMS (ESI⁺) m/z: 507.0 [M+H]⁺.

### Step 6, Compound 139 and 140 Synthesis

Following the synthesis method of Example 6, Step 4, except replacing Step 4, 6-3 with 139-6 (120.0 mg, crude), dimethylamine with 8-1 (41.0 mg, 0.5 mmol) the same synthesis method was applied. The obtained compound was subjected to Prep-MPLC to give 139 (25.0 mg, 43.3 mol, 18.3% yield) and 140 (20.0 mg, 34.6 mol, 14.6% yield), LCMS (ESI⁺) m/z: 578.1 [M+H]⁺.

Compound 139 NMR: ¹H NMR (600 MHz, DMSO-d₆) δ 12.04 (s, 1H), 8.37 (t, J=4.8 Hz, 1H), 6.91 (s, 1H), 6.46 (s, 1H), 4.45-4.42 (m, 1H), 4.34-4.31 (m, 1H), 4.15-4.12 (m, 1H), 4.07-4.04 (m, 1H), 3.95-3.90 (m, 2H), 3.46 (s, 2H), 3.14 (s, 3H), 2.74 (s, 3H), 2.69-2.68 (m, 2H), 2.26 (s, 3H), 2.23-2.22 (m, 1H), 2.11 (s, 3H), 1.62-1.24 (m, 9H).

Compound 140 NMR: ¹H NMR (600 MHz, DMSO-d₆) δ 12.04 (s, 1H), 8.37 (t, J=4.8 Hz, 1H), 6.91 (s, 1H), 6.46 (s, 1H), 4.44-4.42 (m, 1H), 4.33-4.30 (m, 1H), 4.16-4.13 (m, 1H), 4.08-4.05 (m, 1H), 3.95-3.91 (m, 2H), 3.49-3.47 (m, 2H), 3.14 (s, 3H), 2.73-2.71 (m, 5H), 2.27-2.25 (m, 4H), 2.11 (s, 3H), 1.62-1.24 (m, 9H).

### Example 84, Compound 141 and 142 Synthesis

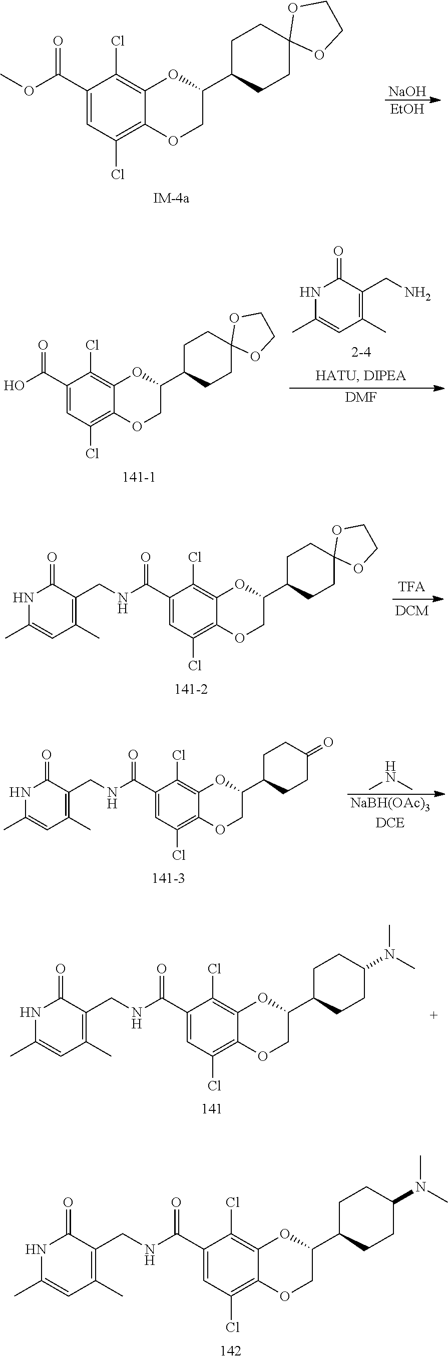

### Step 1, Compound 141-1 Synthesis

Following the synthesis method of Example 6, Step 1, except replacing Step 1, IM-3a with IM-4a (166.0 mg, 0.4 mmol), the same synthesis method was applied, to give Compound 141-1 (160.0 mg, crude), LCMS (ESI⁺) m/z: 389.3 [M+H]⁺.

### Step 2, Compound 141-2 Synthesis

Following the synthesis method of Example 1, Step 3, except replacing Step 3, 1-3 with 141-1 (160.0 mg, crude), 1-4 with 2-4 (125.0 mg, 0.8 mmol), the same synthesis method was applied, to give Compound 141-2 (200.0 mg, crude), LCMS (ESI⁺) m/z: 523.4 [M+H]⁺.

### Step 3, Compound 141-3 Synthesis

Following the synthesis method of Example 3, Step 2, except replacing Step 2, 3-2 with 141-2 (200.0 mg, 0.4 mmol), the same synthesis method was applied, to give Compound 141-3 (180.0 mg, crude), LCMS (ESI⁺) m/z: 479.4 [M+H]⁺.

### Step 4, Compound 141 and 142 Synthesis

Following the synthesis method of Example 6, Step 4, except replacing Step 4, 6-3 with 141-3 (180.0 mg, crude), the same synthesis method was applied. The obtained compound was subjected to Prep-MPLC to give 141 (55.0 mg, 0.1 mmol, 28.8% yield) and 142 (28.5 mg, 56.1 mol, 14.9% yield), LCMS (ESI⁺) m/z: 508.1 [M+H]⁺.

Compound 141 NMR: ¹H NMR (600 MHz, DMSO-d₆) δ 11.48 (s, 1H), 8.30 (t, J=4.8 Hz, 1H), 6.99 (s, 1H), 5.86 (s, 1H), 4.52-4.89 (m, 1H), 4.24 (d, J=4.8 Hz, 2H), 4.19-4.14 (m, 2H), 3.40-3.38 (m, 1H), 2.18 (s, 3H), 2.15 (s, 6H), 2.11 (s, 3H), 2.06-2.03 (m, 1H), 1.89-1.74 (m, 2H), 1.73-1.64 (m, 2H), 1.63-1.53 (m, 2H), 1.44-1.40 (m, 2H).

Compound 142 NMR: ¹H NMR (600 MHz, DMSO-d₆) δ 11.48 (s, 1H), 8.30 (t, J=4.8 Hz, 1H), 7.02 (s, 1H), 5.86 (s, 1H), 4.51 (dd, J=11.4, 1.8 Hz, 1H), 4.24 (d, J=5.4 Hz, 2H), 4.17-4.14 (m, 1H), 4.05-4.03 (m, 1H), 3.41-3.39 (m, 1H), 2.28 (s, 6H), 2.22 (s, 3H), 2.18 (s, 3H), 2.10-2.06 (m, 1H), 1.88-1.82 (m, 2H), 1.81-1.78 (m, 1H), 1.59-1.56 (m, 1H), 1.29-1.16 (m, 4H).

### Example 85, Compound 143 and 144 Synthesis

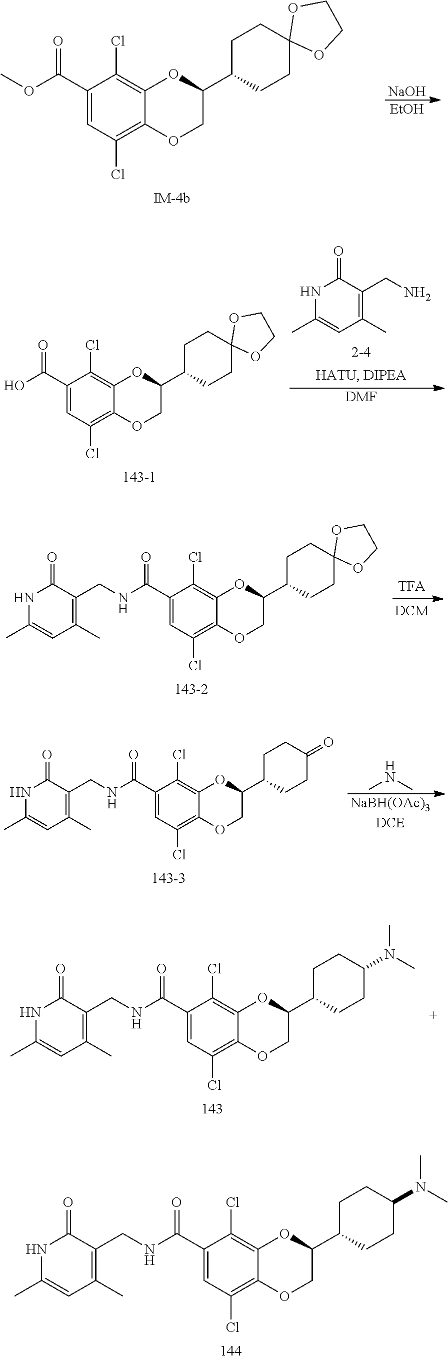

### Step 1, Compound 143-1 Synthesis

Following the synthesis method of Example 6, Step 1, except replacing Step 1, IM-3a with IM-4b (170.0 mg, 0.4 mmol), the same synthesis method was applied, to give Compound 143-1 (160.0 mg, crude), LCMS (ESI⁺) m/z: 389.3 [M+H]⁺.

### Step 2, Compound 143-2 Synthesis

Following the synthesis method of Example 1, Step 3, except replacing Step 3, 1-3 with 143-1 (160.0 mg, crude), 1-4 with 2-4 (125.0 mg, 0.8 mmol), the same synthesis method was applied, to give Compound 143-2 (200.0 mg, crude), LCMS (ESI⁺) m/z: 523.4 [M+H]⁺.

### Step 3, Compound 143-3 Synthesis

Following the synthesis method of Example 3, Step 2, except replacing Step 2, 3-2 with 143-2 (200.0 mg, crude), the same synthesis method was applied, to give Compound 143-3 (180.0 mg, crude), LCMS (ESI⁺) m/z: 479.4 [M+H]⁺.

### Step 4, Compound 143 and 144 Synthesis

Following the synthesis method of Example 6, Step 4, except replacing Step 4, 6-3 with 143-3 (180.0 mg, crude), the same synthesis method was applied. The obtained compound was subjected to Prep-MPLC to give 143 (17.3 mg, 34.0 mol, 9.1% yield) and 144 (37.0 mg, 72.8 mol, 19.4% yield), LCMS (ESI⁺) m/z: 508.1 [M+H]⁺.

Compound 143 NMR: ¹H NMR (600 MHz, DMSO-d₆) δ 11.48 (s, 1H), 8.30 (t, J=4.8 Hz, 1H), 6.99 (s, 1H), 5.86 (s, 1H), 4.52 (dd, J=11.8, 2.2 Hz, 1H), 4.46-4.44 (m, 1H), 4.24 (d, J=4.8 Hz, 2H), 4.19-4.14 (m, 1H), 3.42-3.39 (m, 1H), 2.18 (s, 3H), 2.15 (s, 6H), 2.11 (s, 3H), 2.06-2.04 (m, 1H), 1.87-1.81 (m, 2H), 1.81-1.76 (m, 2H), 1.55 (m, 2H), 1.20-1.11 (m, 2H).

Compound 144 NMR: ¹H NMR (600 MHz, DMSO-d₆) δ 11.48 (s, 1H), 8.30 (t, J=4.8 Hz, 1H), 6.99 (s, 1H), 5.86 (s, 1H), 4.52 (d, J=9.8 Hz, 1H), 4.46-4.44 (m, 1H), 4.24 (d, J=4.8 Hz, 2H), 4.19-4.14 (m, 1H), 3.42-3.39 (m, 1H), 2.18 (s, 3H), 2.15 (s, 6H), 2.11 (s, 3H), 2.06-2.03 (m, 1H), 1.82-1.74 (m, 2H), 1.73-1.64 (m, 2H), 1.61-1.52 (m, 2H), 1.43-1.40 (m, 2H).

### Example 86, Compound 145 and 146 Synthesis

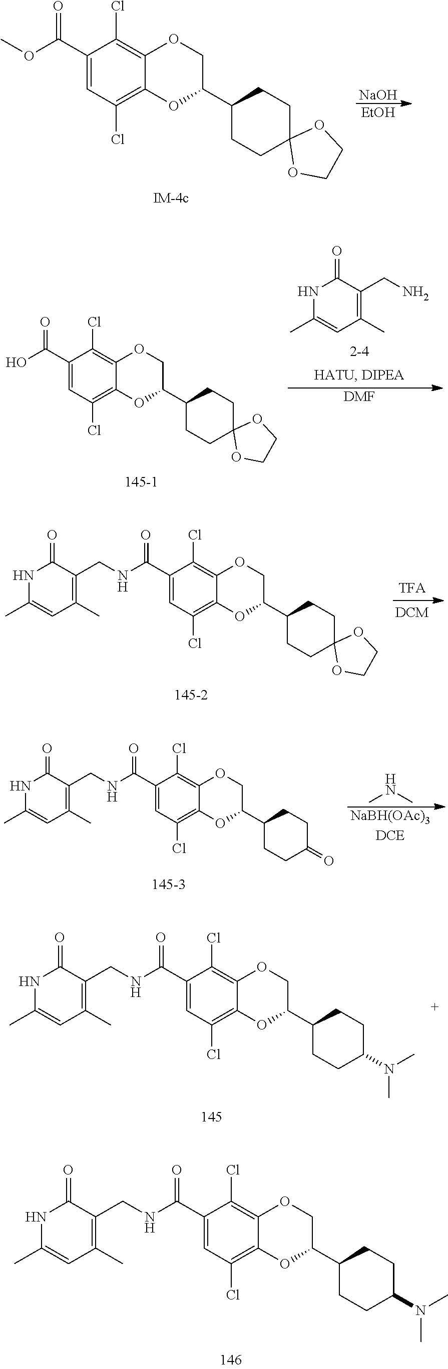

### Step 1, Compound 145-1 Synthesis

Following the synthesis method of Example 6, Step 1, except replacing Step 1, IM-3a with IM-4c (74.0 mg, 0.2 mmol), the same synthesis method was applied, to give Compound 145-1 (70.0 mg, crude), LCMS (ESI⁺) m/z: 389.3 [M+H]⁺.

### Step 2, Compound 145-2 Synthesis

Following the synthesis method of Example 1, Step 3, except replacing Step 3, 1-3 with 145-1 (70.0 mg, crude), 1-4 with 2-4 (33.0 mg, 0.2 mmol), the same synthesis method was applied, to give Compound 145-2 (80.0 mg, crude), LCMS (ESI⁺) m/z: 523.4 [M+H]⁺.

### Step 3, Compound 145-3 Synthesis

Following the synthesis method of Example 3, Step 2, except replacing Step 2, 3-2 with 145-2 (80.0 mg, crude), the same synthesis method was applied, to give Compound 145-3 (65.0 mg, crude), LCMS (ESI⁺) m/z: 479.4 [M+H]⁺.

### Step 4, Compound 145 and 146 Synthesis

Following the synthesis method of Example 6, Step 4, except replacing Step 4, 6-3 with 145-3 (65.0 mg, crude), the same synthesis method was applied. The obtained compound was subjected to Prep-MPLC to give 145 (17.3 mg, 34.0 mol, 25.1% yield) and 146 (37.0 mg, 72.8 mol, 53.7% yield), LCMS (ESI⁺) m/z: 508.1 [M+H]⁺.

Compound 145 NMR: ¹H NMR (600 MHz, DMSO-d₆) δ 11.47 (s, 1H), 8.30 (t, J=4.8 Hz, 1H), 7.01 (s, 1H), 5.86 (s, 1H), 4.52 (dd, J=11.4, 1.8 Hz, 1H), 4.24 (d, J=5.4 Hz, 2H), 4.16-4.13 (m, 1H), 4.06-4.04 (m, 1H), 3.46-3.32 (m, 1H), 2.17 (s, 3H), 2.15 (s, 6H), 2.11 (s, 3H), 2.03-2.01 (m, 1H), 1.85-1.78 (m, 3H), 1.53-1.48 (m, 1H), 1.23-1.14 (m, 4H).

Compound 146 NMR: ¹H NMR (600 MHz, DMSO-d₆) δ 11.47 (s, 1H), 8.30 (t, J=4.8 Hz, 1H), 7.01 (s, 1H), 5.86 (s, 1H), 4.51 (dd, J=10.2, 2.2 Hz, 1H), 4.24 (d, J=5.4 Hz, 2H), 4.19-4.16 (m, 2H), 3.46-3.32 (m, 1H), 2.17 (s, 3H), 2.15 (s, 6H), 2.11 (s, 3H), 2.03-1.99 (m, 1H), 1.87-1.76 (m, 2H), 1.60-1.50 (m, 2H), 1.42-1.40 (m, 2H), 1.21-1.09 (m, 2H).

### Example 87, Compound 147 and 148 Synthesis

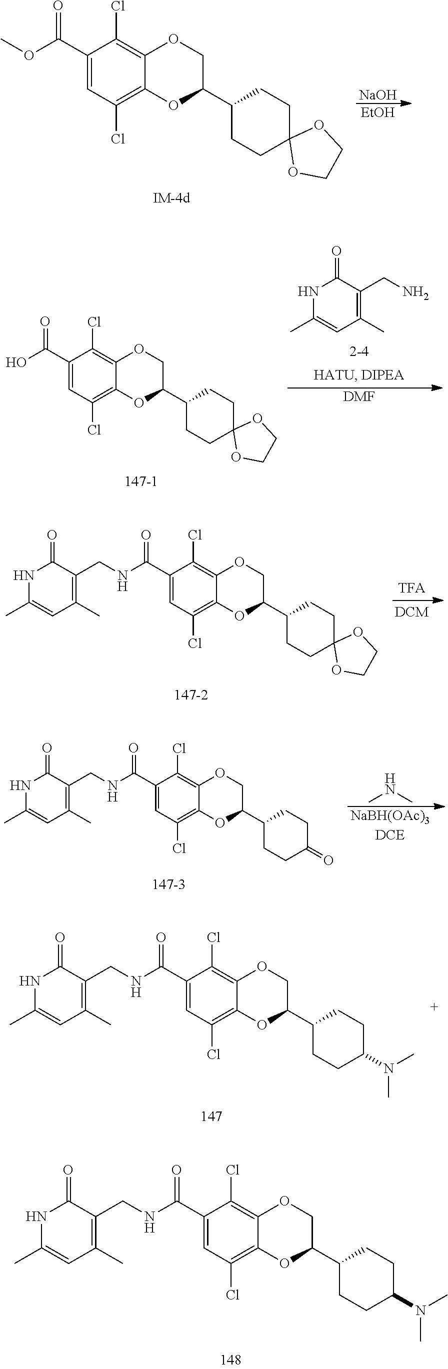

### Step 1, Compound 147-1 Synthesis

Following the synthesis method of Example 6, Step 1, except replacing Step 1, IM-3a with IM-4d (72.0 mg, 0.2 mmol), the same synthesis method was applied, to give Compound 147-1 (65.0 mg, crude), LCMS (ESI⁺) m/z: 389.3 [M+H]⁺.

### Step 2, Compound 147-2 Synthesis

Following the synthesis method of Example 1, Step 3, except replacing Step 3, 1-3 with 147-1 (65.0 mg, crude), 1-4 with 2-4 (30.0 mg, 0.2 mmol), the same synthesis method was applied, to give Compound 147-2 (80.0 mg, crude), LCMS (ESI⁺) m/z: 523.4 [M+H]⁺.

### Step 3, Compound 147-3 Synthesis

Following the synthesis method of Example 3, Step 2, except replacing Step 2, 3-2 with 147-2 (80.0 mg, crude), the same synthesis method was applied, to give Compound 147-3 (60.0 mg, crude), LCMS (ESI⁺) m/z: 479.4 [M+H]⁺.

### Step 4, Compound 147 and 148 Synthesis

Following the synthesis method of Example 6, Step 4, except replacing Step 4, 6-3 with 147-3 (60.0 mg, crude), the same synthesis method was applied. The obtained compound was subjected to Prep-MPLC to give 147 (17.3 mg, 34.0 mol, 27.2% yield) and 148 (37.0 mg, 72.8 mol, 58.2% yield), LCMS (ESI⁺) m/z: 508.1 [M+H]⁺.

Compound 147 NMR: ¹H NMR (600 MHz, DMSO-d₆) δ 11.48 (s, 1H), 8.30 (t, J=4.8 Hz, 1H), 7.03 (s, 1H), 5.87 (s, 1H), 4.52 (dd, J=12.0, 2.4 Hz, 1H), 4.24 (d, J=4.8 Hz, 2H), 4.16-4.13 (m, 1H), 4.06-4.04 (m, 1H), 3.40-3.38 (m, 1H), 2.18 (s, 3H), 2.15 (s, 6H), 2.11 (s, 3H), 2.04-2.00 (m, 1H), 1.89-1.87 (m, 3H), 1.58-1.55 (m, 1H), 1.28-1.15 (m, 4H).

Compound 148 NMR: ¹H NMR (600 MHz, DMSO-d₆) δ 11.48 (s, 1H), 8.29 (t, J=4.8 Hz, 1H), 7.01 (s, 1H), 5.87 (s, 1H), 4.52 (dd, J=11.6, 2.2 Hz, 1H), 4.25 (d, J=4.8 Hz, 2H), 4.19-4.17 (m, 2H), 3.40-3.38 (m, 1H), 2.18 (s, 3H), 2.15 (s, 6H), 2.11 (s, 3H), 2.04-2.00 (m, 1H), 1.89-1.87 (m, 2H), 1.86-1.77 (m, 2H), 1.58-1.55 (m, 2H), 1.48-1.45 (m, 2H).

### Example 88, Compound 149 and 150 Synthesis

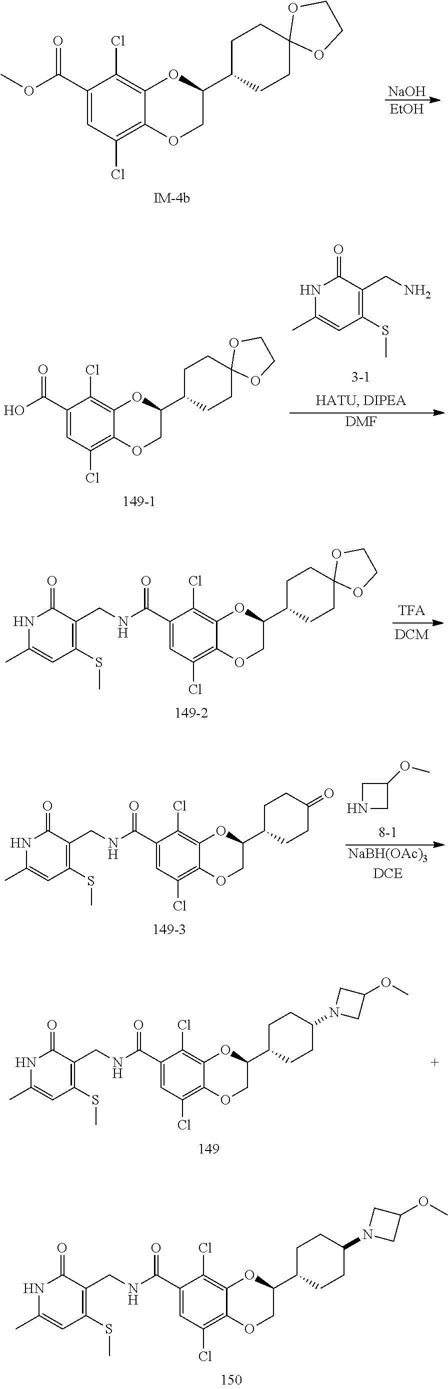

### Step 1, Compound 149-1 Synthesis

Following the synthesis method of Example 6, Step 1, except replacing Step 1, IM-3a with IM-4b (200.0 mg, 0.5 mmol), the same synthesis method was applied, to give Compound 149-1 (200.0 mg, crude), LCMS (ESI⁺) m/z: 389.3 [M+H]⁺.

### Step 2, Compound 149-2 Synthesis

Following the synthesis method of Example 1, Step 3, except replacing Step 3, 1-3 with 149-1 (200.0 mg, crude), 1-4 with 3-1 (113.0 mg, 0.6 mmol), the same synthesis method was applied, to give Compound 149-2 (200.0 mg, crude), LCMS (ESI⁺) m/z: 555.5 [M+H]⁺.

### Step 3, Compound 149-3 Synthesis

Following the synthesis method of Example 3, Step 2, except replacing Step 2, 3-2 with 149-2 (200.0 mg, crude), the same synthesis method was applied, to give Compound 149-3 (200.0 mg, crude), LCMS (ESI⁺) m/z: 511.4 [M+H]⁺.

### Step 4, Compound 149 and 150 Synthesis

Following the synthesis method of Example 6, Step 4, except replacing Step 4, 6-3 with 149-3 (200.0 mg, crude), dimethylamine with 8-1 (103.0 mg, 1.2 mmol), the same synthesis method was applied. The obtained compound was subjected to Prep-MPLC to give 149 (27.3 mg, 46.9 mol, 12.0% yield) and 150 (61.0 mg, 0.1 mmol, 26.8% yield), LCMS (ESI⁺) m/z: 582.2 [M+H]⁺.

Compound 149 NMR: ¹H NMR (600 MHz, DMSO-d₆) δ 11.54 (s, 1H), 8.19 (t, J=4.8 Hz, 1H), 6.98 (s, 1H), 6.08 (s, 1H), 4.50-4.48 (m, 1H), 4.28 (d, J=4.2 Hz, 2H), 4.16-4.13 (m, 1H), 4.07-4.05 (m, 1H), 3.93-3.90 (m, 1H), 3.47 (q, J=6.0 Hz, 2H), 3.14 (s, 3H), 2.67 (s, 2H), 2.45 (s, 3H), 2.24-2.20 (m, 1H), 2.17 (s, 3H), 1.68-1.52 (m, 4H), 1.51-1.28 (m, 5H).

Compound 150 NMR: δ 11.54 (s, 1H), 8.19 (t, J=4.2 Hz, 1H), 7.00 (s, 1H), 6.09 (s, 1H), 4.52 (d, J=9.6 Hz, 1H), 4.28 (d, J=4.2 Hz, 2H), 4.15-4.12 (m, 1H), 4.05-4.02 (m, 1H), 3.91-3.87 (m, 1H), 3.47-3.43 (m, 2H), 3.14 (s, 3H), 2.76-2.73 (m, 2H), 2.46 (s, 3H), 2.17 (s, 3H), 1.98-1.94 (m, 1H), 1.80-1.70 (m, 3H), 1.57-1.53 (m, 1H), 1.31-1.09 (m, 3H), 0.94-0.83 (m, 2H).

### Example 89, Compound 151 and 152 Synthesis

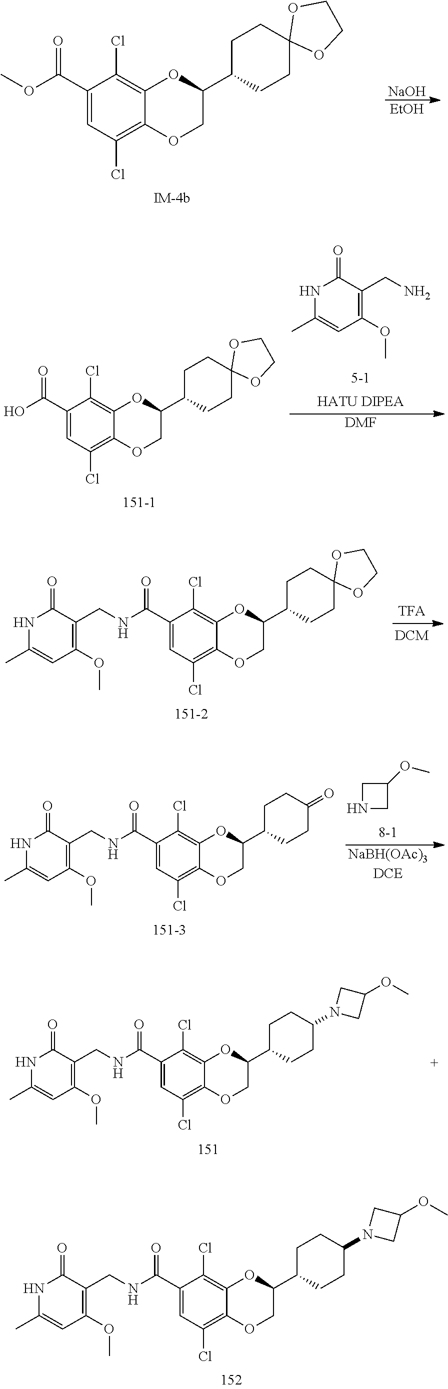

### Step 1, Compound 151-1 Synthesis

Following the synthesis method of Example 6, Step 1, except replacing Step 1, IM-3a with IM-4b (200.0 mg, 0.5 mmol), the same synthesis method was applied, to give Compound 151-1 (200.0 mg, crude), LCMS (ESI) m/z: 389.3 [M+H]⁺)

### Step 2, Compound 151-2 Synthesis

Following the synthesis method of Example 1, Step 3, except replacing Step 3, 1-3 with 151-1 (200.0 mg, crude), 1-4 with 5-1 (100.0 mg, 0.6 mmol), the same synthesis method was applied, to give Compound 151-2 (200.0 mg, crude), LCMS (ESI⁺) m/z: 539.1 [M+H]⁺

### Step 3, Compound 151-3 Synthesis

Following the synthesis method of Example 3, Step 2, except replacing Step 2, 3-2 with 151-2 (200.0 mg, crude), the same synthesis method was applied, to give Compound 151-3 (200.0 mg, crude), LCMS (ESI⁺) m/z: 495.1 [M+H]⁺

### Step 4, Compound 151 and 152 Synthesis

Following the synthesis method of Example 6, Step 4, except replacing Step 4, 6-3 with 151-3 (200.0 mg, crude), dimethylamine with 8-1 (103.0 mg, 1.2 mmol), the same synthesis method was applied. The obtained compound was subjected to Prep-MPLC to give 151 (4.0 mg, 7.1 mol, 1.7% yield) and 152 (11.0 mg, 19.4 mol, 4.8% yield), LCMS (ESI⁺) m/z: 566.2 [M+H]⁺.

Compound 151 NMR: ¹H NMR (600 MHz, DMSO-d₆) δ 11.43 (s, 1H), 8.09 (t, J=4.8 Hz, 1H), 6.99 (s, 1H), 6.09 (s, 1H), 4.52-4.51 (m, 1H), 4.19 (d, J=4.8 Hz, 2H), 4.15-4.02 (m, 2H), 3.94-3.90 (m, 1H), 3.80 (s, 3H), 3.52-3.50 (m, 2H), 3.15 (s, 3H), 2.81-2.79 (m, 2H), 2.18 (s, 3H), 1.99-1.96 (m, 2H), 1.80-1.72 (m, 3H), 1.57-1.56 (m, 1H), 1.26-1.14 (m, 3H), 0.96-0.84 (m, 2H).

Compound 152 NMR: ¹H NMR (600 MHz, DMSO-d₆) δ 11.43 (s, 1H), 8.09 (t, J=4.2 Hz, 1H), 6.98 (s, 1H), 6.09 (s, 1H), 4.50 (d, J=10.8 Hz, 1H), 4.19 (d, J=4.2 Hz, 2H), 4.16-4.12 (m, 1H), 4.07-4.04 (m, 1H), 3.94-3.90 (m, 1H), 3.80 (s, 3H), 3.46-3.44 (m, 2H), 3.14 (s, 3H), 2.68-2.66 (m, 2H), 2.23-2.21 (m, 1H), 2.18 (s, 3H), 1.65-1.57 (m, 4H), 1.51-1.39 (m, 5H).

### Example 90, Compound 153 and 154 Synthesis

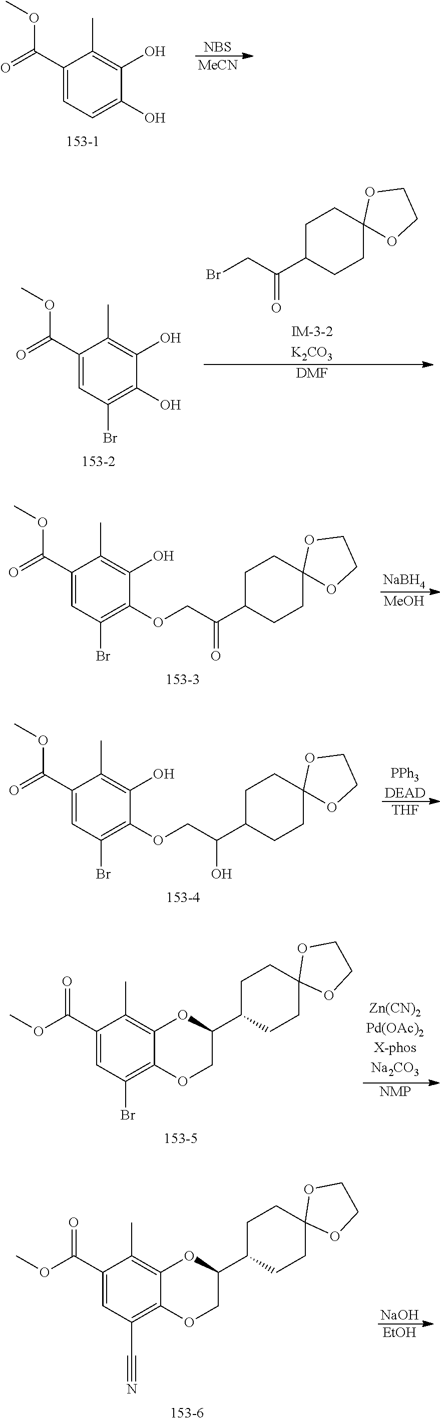

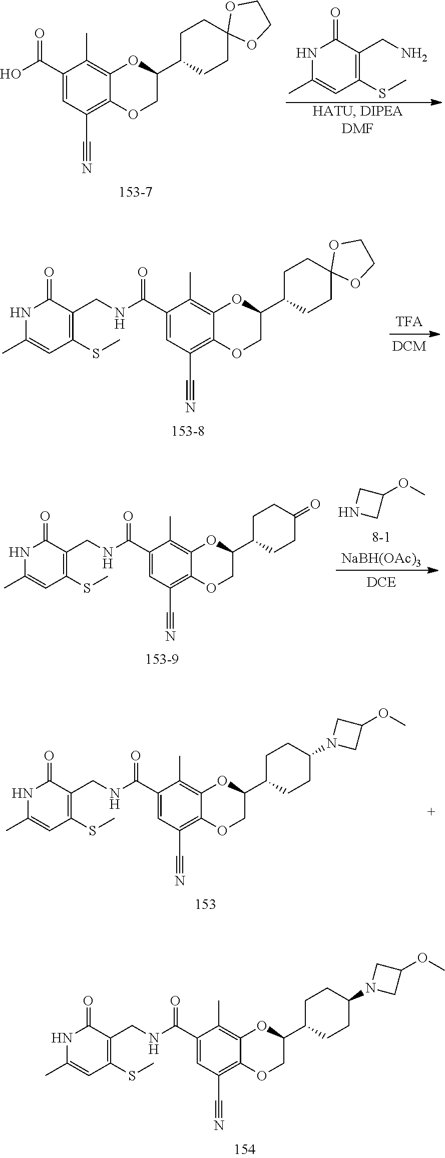

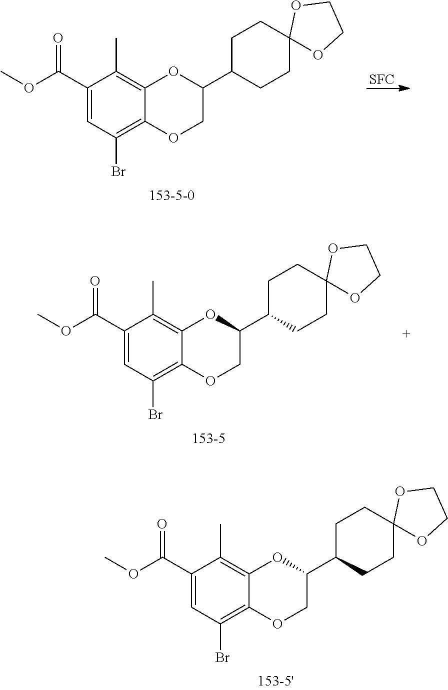

### Step 1, Compound 153-2 Synthesis

A single-necked flask was added with substrate 153-1 (5.4 g, 29.6 mmol). The mixture was dissolved with ACN (50 mL) to dissolve, and then added with NBS (5.8 g, 32.0 mmol), at 25° C. stirred to react for 3 hours, with LC-MS monitoring. After the completion of the reaction, the reaction mixture was condensated under reduced pressure, and then extracted with water and EA solution three times. The organic phase was combined, washed with saturated saline solution, and dried with anhydrous sodium sulfate and then condensated under reduced pressure to give the crude product 153-2 (9.8 g, crude). LCMS (ESI⁺) m/z: 261.0 [M+H]⁺

### Step 2, Compound 153-3 Synthesis

Following the synthesis method of Example 2, Step 1, except replacing Step 1, IM-2 with IM-3-2 (8.5 g, 32.3 mmol), 2-1 with 153-2 (7.7 g, 29.5 mmol), the same synthesis method was applied, to give Compound 153-3 (12.6 g, crude). LCMS (ESI⁺) m/z: 443.3 [M+H]⁺

### Step 3, Compound 153-4 Synthesis

Following the synthesis method of Example IM-3, Step 3, except replacing Step 3, IM-3-4 with 153-3 (12.6 g, crude), the same synthesis method was applied, to give Compound 153-4 (7.0 g, 15.7 mmol, 55.10. yield). LCMS (ESI⁺)) m/z: 445.3 [M+H]⁺

### Step 4, Compound 153-5 Synthesis

Following the synthesis method of Example IM-3, Step 4, except replacing Step 4, IM-3-5 with 153-4 (7.0 g, 15.7 mmol), the same synthesis method was applied, to give Compound 153-5-0 which was separated by SFC to give Compound 153-5 (2.4 g, 5.6 mmol, 35.8% yield) and Compound 153-5′(2.4 g, 5.5 mmol, 35.8% yield). LCMS (ESI⁺) m/z: 427.3 [M+H]⁺

### Step 5, Compound 153-6 Synthesis

A single-necked flask was added with substrate 153-5 (213.7 mg, 0.5 mmol). The mixture was dissolved with NMP (5 mL) to dissolve, and then added with Zn(CN)₂ (117.7 mg, 1.0 mmol), Pd(OAc)₂ (22.5 mg, 0.1 mmol), X-phos (95.5 mg, 0.2 mmol) and Na₂CO₃ (53.0 mg, 0.5 mmol), and under nitrogen protection and at 100° C. stirred to react for 2 hours, with LC-MS monitoring. After the completion of the reaction, the reaction mixture was cooled to room temperature, and condensated under reduced pressure, and then purified by medium pressure liquid chromatography to give the product 153-6 (165.0 mg, 0.4 mmol, 88.4% yield). LCMS (ESI⁺) m/z: 373.4 [M+H]⁺

### Step 6, Compound 153-7 Synthesis

Following the synthesis method of Example 6, Step 1, except replacing Step 1, IM-3a with 153-6 (165.0 mg, 0.4 mmol), the same synthesis method was applied, to give Compound 153-7 (159.0 mg, crude), LCMS (ESI⁺) m/z: 359.3 [M+H]⁺.

### Step 7, Compound 153-8 Synthesis

Following the synthesis method of Example 1, Step 3, except replacing Step 3, 1-3 with 153-7 (159.0 mg, crude), 1-4 with 3-1 (150.0 mg, 0.9 mmol), the same synthesis method was applied, to give Compound 153-8 (255.0 mg, crude), LCMS (ESI⁺) m/z: 525.6 [M+H]⁺.

### Step 8, Compound 153-9 Synthesis

Following the synthesis method of Example 3, Step 2, except replacing Step 2, 3-2 with 153-8 (255.0 mg, crude), the same synthesis method was applied, to give Compound 153-9 (122.0 mg, crude), LCMS (ESI⁺) m/z: 481.5 [M+H]⁺.

### Step 9, Compound 153 and 154 Synthesis

Following the synthesis method of Example 6, Step 4, except replacing Step 4, 6-3 with 153-9 (48.0 mg, 0.1 mmol), dimethylamine with 8-1 (17.0 mg, 0.2 mmol), the same synthesis method was applied. The obtained compound was subjected to Prep-MPLC to give 153 (8.0 mg, 14.5 mol, 14.5% yield) and 154 (14.0 mg, 25.3 mol, 25.4% yield), LCMS (ESI⁺) m/z: 553.4 [M+H]⁺.

Compound 153 NMR: ¹H NMR (600 MHz, DMSO-d₆) δ 11.53 (s, 1H), 8.10 (t, J=4.2 Hz, 1H), 7.17 (s, 1H), 6.08 (s, 1H), 4.54-4.52 (m, 1H), 4.28 (d, J=4.2 Hz, 2H), 4.15-4.12 (m, 1H), 3.98-3.96 (m, 1H), 3.89-3.87 (m, 1H), 3.45-3.42 (m, 2H), 3.13 (s, 3H), 2.72-2.70 (m, 2H), 2.45 (s, 3H), 2.21 (s, 3H), 2.17 (s, 3H), 1.95-1.88 (m, 2H), 1.77-1.71 (m, 3H), 1.56-1.55 (m, 1H), 1.19-1.14 (m, 2H), 0.89-0.85 (m, 2H).

Compound 154 NMR: ¹H NMR (600 MHz, DMSO-d₆) δ 11.53 (s, 1H), 8.10 (t, J=4.2 Hz, 1H), 7.16 (s, 1H), 6.08 (s, 1H), 4.51-4.50 (m, 1H), 4.28 (d, J=4.2 Hz, 2H), 4.16-4.13 (m, 1H), 4.01-3.99 (m, 1H), 3.92-3.89 (m, 1H), 3.45-3.44 (m, 2H), 3.14 (s, 3H), 2.68-2.66 (m, 2H), 2.45 (s, 3H), 2.21 (s, 3H), 2.17 (s, 3H), 1.66-1.20 (m, 10H).

### Example 91, Compound 155 and 156 Synthesis

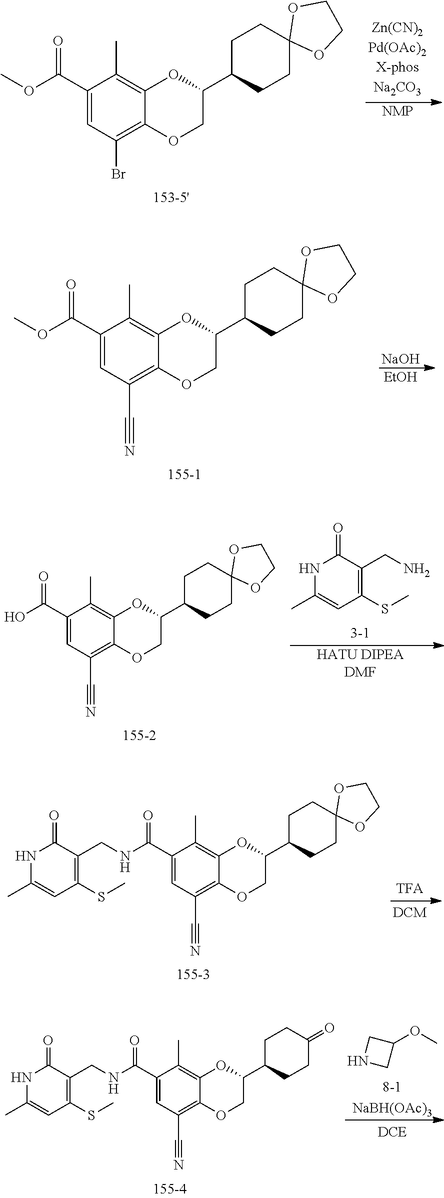

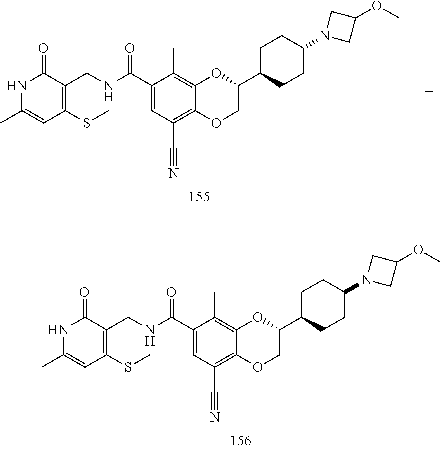

### Step 1, Compound 155-1 Synthesis

A single-necked flask was added with substrate 153-5′ (214.0 mg, 0.5 mmol). The mixture was dissolved with NMP (5 mL) to dissolve, and then added with Zn(CN)₂ (117.7 mg, 1.0 mmol), Pd(OAc)₂ (22.5 mg, 0.1 mmol), X-phos (95.5 mg, 0.2 mmol) and Na₂CO₃ (53.0 mg, 0.5 mmol), and under nitrogen protection and at 100° C. stirred to react for 2 hours, with LC-MS monitoring. After the completion of the reaction, the reaction mixture was cooled to room temperature, and condensated under reduced pressure, and then purified by medium pressure liquid chromatography to give the product 155-1 (165.0 mg, 0.4 mmol, 88.4%). LCMS (ESI+) m/z: 373.4 [M+H]⁺

### Step 2, Compound 155-2 Synthesis

Following the synthesis method of Example 6, Step 1, except replacing Step 1, IM-3a with 155-1 (165.0 mg, 0.4 mmol), the same synthesis method was applied, to give Compound 155-2 (159.0 mg, crude), LCMS (ESI+) m/z: 359.3 [M+H]⁺.

### Step 3, Compound 155-3 Synthesis

Following the synthesis method of Example 1, Step 3, except replacing Step 3, 1-3 with 155-2 (159.0 mg, crude), 1-4 with 3-1 (150.0 mg, 0.9 mmol), the same synthesis method was applied, to give Compound 155-3 (255.0 mg, crude), LCMS (ESI⁺) m/z: 525.6 [M+H]⁺.

### Step 4, Compound 155-4 Synthesis

Following the synthesis method of Example 3, Step 2, except replacing Step 2, 3-2 with 155-3 (255.0 mg, crude), the same synthesis method was applied, to give Compound 155-4 (122.0 mg, crude), LCMS (ESI⁺) m/z: 481.5 [M+H]⁺.

### Step 5, Compound 155 and 156 Synthesis

Following the synthesis method of Example 6, Step 4, except replacing Step 4, 6-3 with 155-4 (48.0 mg, 0.1 mmol), dimethylamine with 8-1 (17.0 mg, 0.2 mmol), the same synthesis method was applied. The obtained compound was subjected to Prep-MPLC to give 155 (8.0 mg, 14.5 mol, 14.5% yield) and 156 (14.0 mg, 25.3 mol, 25.4% yield), LCMS (ESI⁺) m/z: 553.3 [M+H]⁺.

Compound 155 NMR: ¹H NMR (600 MHz, DMSO-d₆) δ 11.53 (s, 1H), 8.12 (t, J=4.2 Hz, 1H), 7.16 (s, 1H), 6.08 (s, 1H), 4.55-4.50 (m, 1H), 4.28 (d, J=4.2 Hz, 2H), 4.17-4.11 (m, 1H), 4.03-3.97 (m, 1H), 3.94-3.89 (m, 1H), 3.47-3.41 (m, 2H), 3.13 (s, 3H), 2.70-2.67 (m, 2H), 2.45 (s, 3H), 2.21 (s, 3H), 2.17 (s, 3H), 1.57-1.38 (m, 10H).

Compound 156 NMR: ¹H NMR (600 MHz, DMSO-d₆) δ 11.53 (s, 1H), 8.14 (t, J=4.2 Hz 1H), 7.16 (s, 1H), 6.08 (s, 1H), 4.54-4.50 (m, 1H), 4.29 (d, J=4.6 Hz, 2H), 4.18-4.03 (m, 1H), 3.98-3.96 (m, 1H), 3.90-3.88 (m, 1H), 3.46-3.42 (m, 2H), 3.13 (s, 3H), 2.72-2.68 (m, 2H), 2.45 (s, 3H), 2.21 (s, 3H), 2.17 (s, 3H), 1.95-1.84 (m, 2H), 1.79-1.70 (m, 3H), 1.58-1.52 (m, 1H), 1.19-1.12 (m, 2H), 0.91-0.82 (m, 2H).

### Example 92, Compound 157 and 158 Synthesis

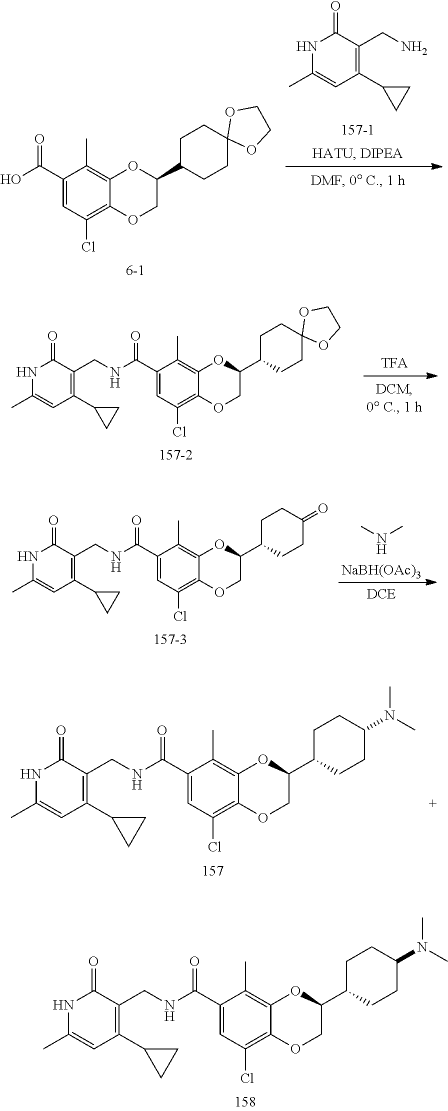

### Step 1, Compound 157-2 Synthesis

Following the synthesis method of Example 1, Step 3, except replacing Step 3, 1-3 with 6-1 (73.0 mg, 0.2 mmol), 1-4 with 157-1 (107.0 mg, 0.6 mmol), the same synthesis method was applied, to give Compound 157-2 (248.0 mg, crude), LCMS (ESI⁺) m/z: 543.1 [M+H]⁺.

### Step 2, Compound 157-3 Synthesis

Following the synthesis method of Example 3, Step 2, except replacing Step 2, 3-2 with 157-2 (248.0 mg, crude), the same synthesis method was applied, to give Compound 157-3 (150.0 mg, crude), LCMS (ESI⁺) m/z: 499.0 [M+H]⁺.

### Step 3, Compound 157 and 158 Synthesis

Following the synthesis method of Example 6, Step 4, except replacing Step 4, 6-3 with 157-3 (150.0 mg, crude), the same synthesis method was applied. The obtained compound was subjected to Prep-MPLC to give 157 (22.0 mg, 42.8 mol, 13.8% yield) and 158 (28.0 mg, 54.5 mol, 17.6% yield), LCMS (ESI⁺) m/z: 514.2 [M+H]⁺.

Compound 157 NMR: ¹H NMR (600 MHz, DMSO-d₆) δ 11.48 (s, 1H), 8.10 (t, J=4.8 Hz, 1H), 6.88 (s, 1H), 5.69 (s, 1H), 4.48-4.43 (m, 1H), 4.21 (d, J=4.8 Hz, 2H), 4.09-4.03 (m, 1H), 4.02-3.88 (m, 1H), 2.19-2.13 (m, 9H), 2.11 (s, 3H), 2.03-1.98 (m, 1H), 1.85-1.81 (m, 2H), 1.80-1.74 (m, 2H), 1.59-1.50 (m, 1H), 1.35-1.10 (m, 5H), 0.95-0.90 (m, 2H), 0.79-0.74 (m, 2H).

Compound 158 NMR: ¹H NMR (600 MHz, DMSO-d₆) δ 11.49 (s, 1H), 8.11 (t, J=4.8 Hz, 1H), 6.88 (s, 1H), 5.69 (s, 1H), 4.44 (d, J=10.4 Hz, 1H), 4.21 (d, J=4.8 Hz, 2H), 4.10-4.03 (m, 2H), 2.18-2.12 (m, 9H), 2.11 (s, 3H), 2.05-2.01 (m, 1H), 1.82-1.74 (m, 3H), 1.72-1.63 (m, 2H), 1.60-1.52 (m, 2H), 1.46-1.36 (m, 3H), 0.96-0.90 (m, 2H), 0.79-0.74 (m, 2H).

### Example 93, Compound 159 and 160 Synthesis

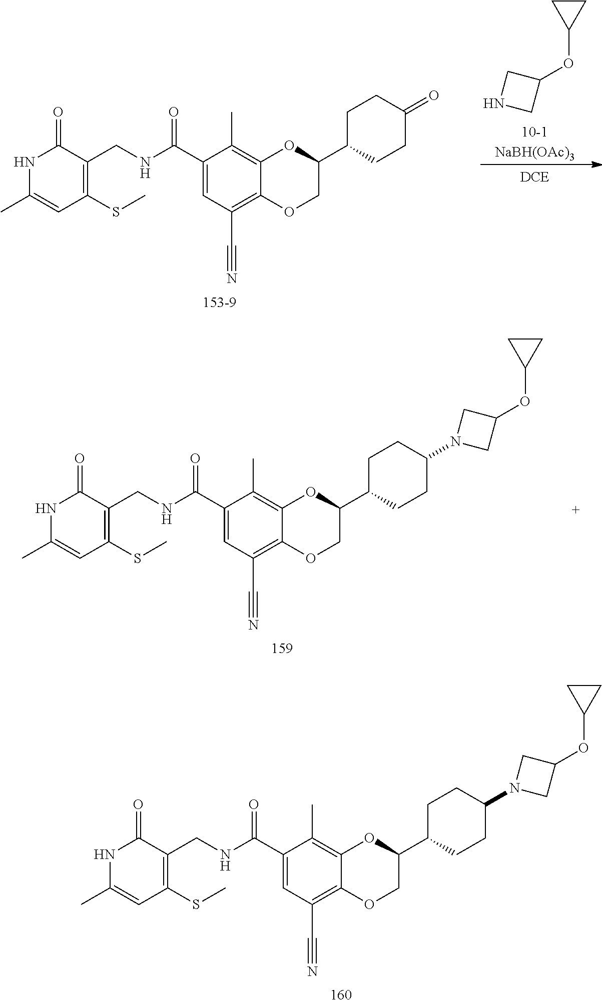

### Step 1, Compound 159 and 160 Synthesis

Following the synthesis method of Example 6, Step 4, except replacing Step 4, 6-3 with 153-9 (67.0 mg, 0.1 mmol), dimethylamine with 10-1 (32.0 mg, 0.3 mmol), the same synthesis method was applied. The obtained compound was subjected to Prep-MPLC to give 159 (8.0 mg, 13.8 mol, 9.9% yield) and 160 (16.0 mg, 27.6 mol, 19.9% yield), LCMS (ESI⁺) m/z: 579.3 [M+H]⁺.

Compound 159 NMR: ¹H NMR (600 MHz, DMSO-d₆) δ 11.53 (s, 1H), 8.09 (t, J=4.8 Hz, 1H), 7.17 (s, 1H), 6.08 (s, 1H), 4.57-4.48 (m, 1H), 4.28 (d, J=4.2 Hz, 2H), 4.17-4.11 (m, 1H), 4.09-4.03 (m, 1H), 3.99-3.94 (m, 1H), 3.50-3.42 (m, 2H), 3.21-3.17 (m, 1H), 2.77-2.71 (m, 2H), 2.45 (s, 3H), 2.21 (s, 3H), 2.17 (s, 3H), 1.96-1.84 (m, 2H), 1.79-1.68 (m, 3H), 1.60-1.51 (m, 1H), 1.21-1.11 (m, 2H), 0.92-0.81 (m, 2H), 0.47-0.42 (m, 2H), 0.41-0.36 (m, 2H).

Compound 160 NMR: ¹H NMR (600 MHz, DMSO-d₆) δ 11.53 (s, 1H), 8.12 (t, J=4.2 Hz, 1H), 7.16 (s, 1H), 6.08 (s, 1H), 4.53-4.47 (m, 1H), 4.28 (d, J=4.8 Hz, 2H), 4.16-4.12 (m, 1H), 4.11-4.07 (m, 1H), 4.04-3.97 (m, 1H), 3.50-3.44 (m, 2H), 3.24-3.18 (m, 1H), 2.73-2.66 (m, 2H), 2.45 (s, 3H), 2.21 (s, 3H), 2.17 (s, 3H), 1.65-1.24 (m, 10H), 0.48-0.43 (m, 2H), 0.41-0.37 (m, 2H).

### Example 94, Compound 161 and 162 Synthesis

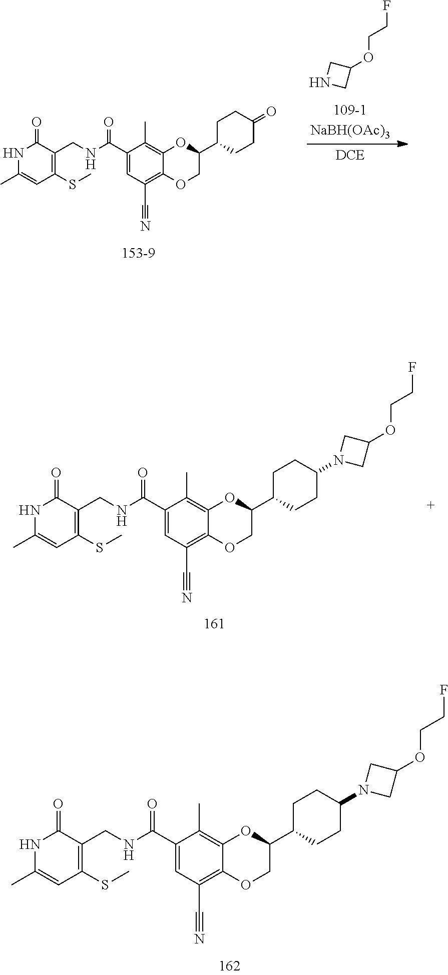

### Step 1, Compound 161 and 162 Synthesis

Following the synthesis method of Example 6, Step 4, except replacing Step 4, 6-3 with 153-9 (96.0 mg, 0.2 mmol), dimethylamine with 109-1 (48.0 mg, 0.4 mmol), the same synthesis method was applied. The obtained compound was subjected to Prep-MPLC to give 161 (8.0 mg, 13.7 μmol, 6.9% yield) and 162 (16.0 mg, 27.4 mol, 13.8% yield), LCMS (ESI⁺) m/z: 585.4 [M+H]⁺.

Compound 161 NMR: ¹H NMR (600 MHz, DMSO-d₆) δ 11.53 (s, 1H), 8.15 (t, J=4.2 Hz, 1H), 7.17 (s, 1H), 6.08 (s, 1H), 4.57-4.50 (m, 2H), 4.47-4.41 (m, 1H), 4.28 (d, J=4.2 Hz, 2H), 4.18-4.11 (m, 1H), 4.08-4.00 (m, 1H), 4.00-3.94 (m, 1H), 3.60-3.55 (m, 1H), 3.55-3.50 (m, 1H), 3.50-3.44 (m, 2H), 2.80-2.71 (m, 2H), 2.45 (s, 3H), 2.21 (s, 3H), 2.17 (s, 3H), 1.98-1.86 (m, 2H), 1.81-1.69 (m, 3H), 1.60-1.50 (m, 1H), 1.22-1.10 (m, 2H), 0.96-0.80 (m, 2H).

Compound 162 NMR: ¹H NMR (600 MHz, DMSO-d₆) δ 11.53 (s, 1H), 8.14 (t, J=4.2 Hz, 1H), 7.17 (s, 1H), 6.08 (s, 1H), 4.59-4.49 (m, 2H), 4.47-4.43 (m, 1H), 4.28 (d, J=4.4 Hz, 2H), 4.20-4.12 (m, 1H), 4.11-4.00 (m, 2H), 3.59 (m, 1H), 3.54 (m, 1H), 3.48 (m, 2H), 2.70 (m, 2H), 2.45 (s, 3H), 2.21 (s, 3H), 2.17 (s, 3H), 1.68-1.23 (m, 10H).

### Example 95, Compound 163 and 164 Synthesis

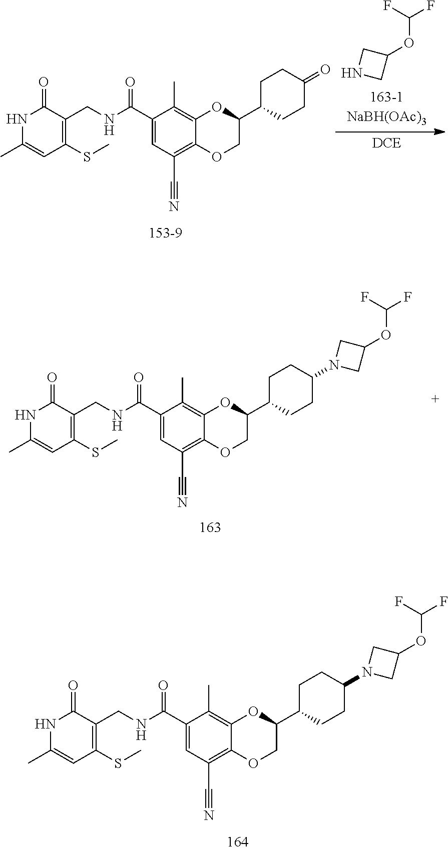

### Step 1, Compound 163 and 164 Synthesis

Following the synthesis method of Example 6, Step 4, except replacing Step 4, 6-3 with 153-9 (72.0 mg, 0.15 mmol), dimethylamine with 163-1 (37.0 mg, 0.3 mmol), the same synthesis method was applied. The obtained compound was subjected to Prep-MPLC to give 163 (6.0 mg, 10.2 mol, 6.8% yield) and 164 (23.0 mg, 39.1 mol, 26.1% yield), LCMS (ESI⁺) m/z: 589.2 [M+H]⁺.

Compound 163 NMR: ¹H NMR (600 MHz, DMSO-d₆) δ 11.53 (s, 1H), 8.14 (t, J=4.2 Hz, 1H), 7.17 (s, 1H), 6.80-6.78 (m, 1H), 6.08 (s, 1H), 4.68-4.59 (m, 1H), 4.56-4.49 (m, 1H), 4.28 (d, J=4.4 Hz, 2H), 4.19-4.11 (m, 1H), 4.01-3.95 (m, 1H), 3.56-3.48 (m, 2H), 2.93-2.89 (m, 2H), 2.45 (s, 3H), 2.21 (s, 3H), 2.17 (s, 3H), 1.97-1.92 (m, 2H), 1.80-1.69 (m, 3H), 1.27-1.11 (m, 3H), 0.94-0.82 (m, 2H).

Compound 164 NMR: ¹H NMR (600 MHz, DMSO-d₆) δ 11.53 (s, 1H), 8.10 (t, J=4.2 Hz, 1H), 7.17 (s, 1H), 6.79-6.54 (m, 1H), 6.08 (s, 1H), 4.70-4.62 (m, 1H), 4.52-4.49 (m, 1H), 4.28 (d, J=4.2 Hz, 2H), 4.18-4.12 (m, 1H), 4.03-3.96 (m, 1H), 3.58-3.48 (m, 2H), 2.91-2.82 (m, 2H), 2.45 (s, 3H), 2.21 (s, 3H), 2.17 (s, 3H), 1.68-1.30 (m, 10H).

### Example 96, Compound 165 and 166 Synthesis

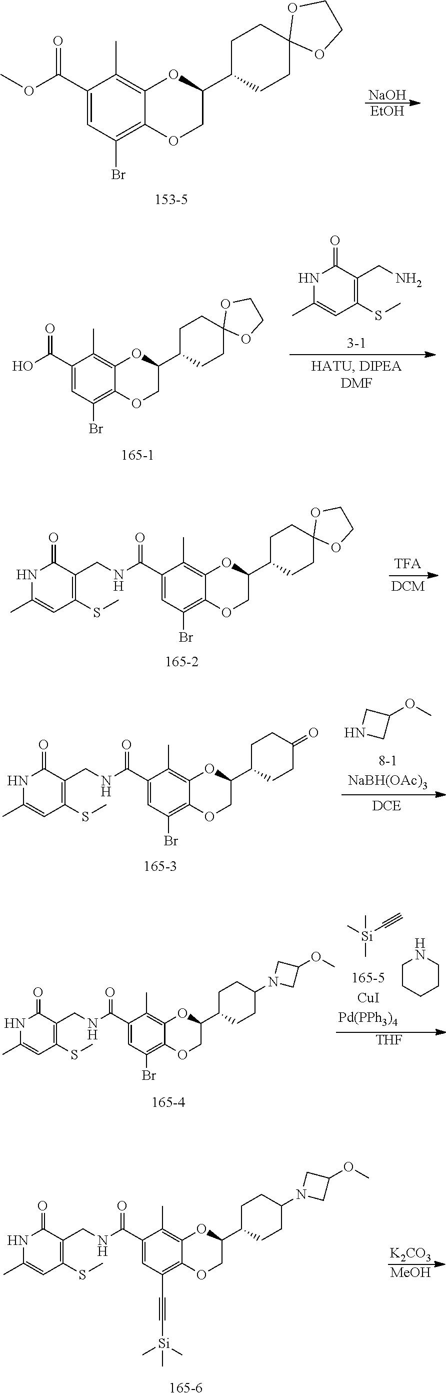

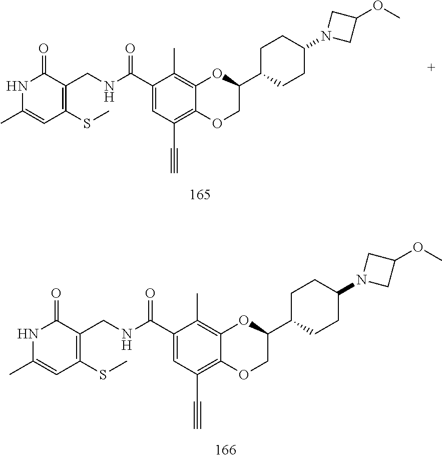

### Step 1, Compound 165-1 Synthesis

Following the synthesis method of Example 6, Step 1, except replacing Step 1, IM-3a with 153-5 (214.0 mg, 0.5 mmol), the same synthesis method was applied, to give Compound 165-1 (210.0 mg, crude), LCMS (ESI⁺) m/z: 413.6 [M+H]⁺.

### Step 2, Compound 165-2 Synthesis

Following the synthesis method of Example 1, Step 3, except replacing Step 3, 1-3 with 165-1 (210.0 mg, crude), 1-4 with 3-1 (184.0 mg, 1.0 mmol), the same synthesis method was applied, to give Compound 165-2 (352.0 mg, crude), LCMS (ESI⁺) m/z: 579.5 [M+H]⁺.

### Step 3, Compound 165-3 Synthesis

Following the synthesis method of Example 3, Step 2, except replacing Step 2, 3-2 with 165-2 (352.0 mg, crude), the same synthesis method was applied, to give Compound 165-3 (453.0 mg, crude), LCMS (ESI⁺) m/z: 535.5 [M+H]⁺.

### Step 4, Compound 165-4 Synthesis

Following the synthesis method of Example 6, Step 4, except replacing Step 4, 6-3 with 165-3 (453.0 mg, crude), dimethylamine with 8-1 (147.0 mg, 1.7 mmol), the same synthesis method was applied, to give Compound 165-4 (283.0 mg, 0.5 mmol, 55.1% yield), LCMS (ESI⁺) m/z: 606.6 [M+H]⁺

### Step 5, Compound 165-6 Synthesis

A single-necked flask was added with substrate 165-4 (283.0 mg, 0.5 mmol). The mixture was dissolved with THE (2 mL) and piperidine (2.3 mL) to dissolve, and then added with 165-5 (98.2 mg, 1.0 mmol), CuI (44.0 mg, 0.2 mmol) and Pd(PPh₃)₄ (270.0 mg, 0.2 mmol), under nitrogen protection heated to stirred at 50° C. to react for 48 hours, with LC-MS monitoring. After the completion of the reaction, the reaction mixture was cooled to room temperature, the reaction mixture was condensated under reduced pressure, and then extracted with water and EA solution three times. The organic phase was combined, washed with saturated saline solution, and dried with anhydrous sodium sulfate and then condensated under reduced pressure, and then purified by medium pressure liquid chromatography to give the product 165-6 (150.0 mg, 0.2 mmol, 51.5% yield). LCMS (ESI⁺) m/z: 623.9 [M+H]⁺

### Step 6, Compound 165 and 166 Synthesis

A single-necked flask was added with substrate 165-6 (150.0 mg, 0.2 mmol). The mixture was dissolved with MeOH (2 mL) to dissolve, and then added with K₂CO₃ (69.0 mg, 0.5 mmol), at 25° C. stirred to react for 1 hour, with LC-MS monitoring. After the completion of the reaction, the reaction mixture was condensated under reduced pressure, and then purified by medium pressure liquid chromatography to give the product 165 (18.0 mg, 0.03 mmol, 13.6% yield) and 166 (26.0 mg, 0.05 mmol, 19.6% yield). LCMS (ESI⁺) m/z: 552.3 [M+H]⁺

Compound 165 NMR: ¹H NMR (600 MHz, DMSO-d₆) δ 11.51 (s, 1H), 7.99 (t, J=4.2 Hz, 1H), 6.90 (s, 1H), 6.07 (s, 1H), 4.46-4.40 (m, 1H), 4.26 (d, J=4.6 Hz, 2H), 4.21 (s, 1H), 4.05-3.99 (m, 1H), 3.93-3.83 (m, 2H), 3.45-3.42 (m, 2H), 3.13 (s, 3H), 2.72-2.69 (m, 2H), 2.45 (s, 3H), 2.16 (m, 6H), 1.96-1.91 (m, 1H), 1.90-1.84 (m, 1H), 1.78-1.69 (m, 3H), 1.56-1.48 (m, 1H), 1.21-1.10 (m, 2H), 0.94-0.80 (m, 2H).

Compound 166 NMR: ¹H NMR (400 MHz, DMSO-d₆) δ 11.51 (s, 1H), 8.02 (t, J=4.4 Hz, 1H), 6.89 (s, 1H), 6.13-6.05 (m, 1H), 4.42-4.35 (m, 1H), 4.27 (d, J=4.4 Hz, 2H), 4.21 (s, 1H), 4.08-3.97 (m, 1H), 3.95-3.86 (m, 2H), 3.49-3.40 (m, 2H), 3.14 (s, 3H), 2.70-2.63 (m, 2H), 2.45 (s, 3H), 2.18-2.13 (m, 6H), 1.67-1.20 (m, 10H).

### Example 97, Compound 167 and 168 Synthesis

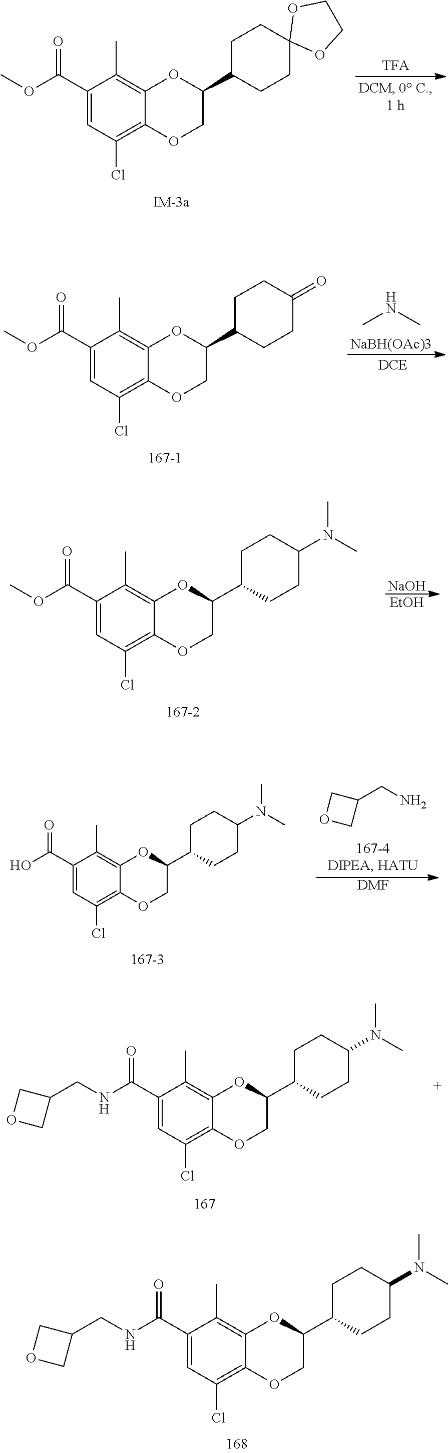

### Step 1, Compound 167-1 Synthesis

Following the synthesis method of Example 3, Step 2, except replacing Step 2, 3-2 with IM-3a (250.0 mg, 0.7 mmol), the same synthesis method was applied, to give Compound 167-1 (170.0 mg, crude), LCMS (ESI⁺) m/z: 338.8 [M+H]⁺.

### Step 2, Compound 167-2 Synthesis

Following the synthesis method of Example 6, Step 4, except replacing Step 4, 6-3 with 167-1 (170.0 mg, crude), the same synthesis method was applied, to give Compound 167-2 (116.0 mg, crude), LCMS (ESI⁺) m/z: 367.9 [M+H]⁺.

### Step 3, Compound 167-3 Synthesis

Following the synthesis method of Example 6, Step 1, except replacing Step 1, IM-3a with 167-2 (116.0 mg, crude), the same synthesis method was applied, to give Compound 167-3 (100.0 mg, crude), LCMS (ESI⁺) m/z: 353.8 [M+H]⁺.

### Step 4, Compound 167 and 168 Synthesis

Following the synthesis method of Example 1, Step 3, except replacing Step 3, 1-3 with 167-3 (100.0 mg, crude), 1-4 with 167-4 (49.0 mg, 0.6 mmol), the same synthesis method was applied.

The obtained compound was subjected to Prep-MPLC to give Compound 167 (13.0 mg, 30.7 mol, 10.9% yield) and Compound 168 (7.5 mg, 17.7 mol, 6.3% yield), LCMS (ESI⁺) m/z: 423.1 [M+H]⁺.

Compound 167 NMR: ¹H NMR (400 MHz, DMSO-d₆) 8.38 (t, J=6.0 Hz, 1H), 6.89 (s, 1H), 4.64-4.60 (m, 2H), 4.46-4.42 (m, 1H), 4.32 (t, J=6.0 Hz, 2H), 4.11-4.04 (m, 2H), 3.48-3.44 (m, 2H), 3.16-3.09 (m, 1H), 2.17-2.15 (m, 6H), 2.12 (s, 3H), 1.82-1.39 (m, 10H).

Compound 168 NMR: ¹H NMR (400 MHz, DMSO-d₆) 8.38 (t, J=6.0 Hz, 1H), 6.89 (s, 1H), 4.64-4.60 (m, 2H), 4.50-4.46 (m, 1H), 4.32 (t, J=6.0 Hz, 2H), 4.11-4.06 (m, 1H), 3.94-3.90 (m, 1H), 3.48-3.44 (m, 2H), 3.16-3.09 (m, 1H), 2.17-2.15 (m, 6H), 2.13 (s, 3H), 2.12-1.93 (m, 1H), 1.88-1.77 (m, 3H), 1.58-1.52 (m, 1H), 1.26-1.12 (m, 5H).

### Example 98, Compound 169 and 170 Synthesis

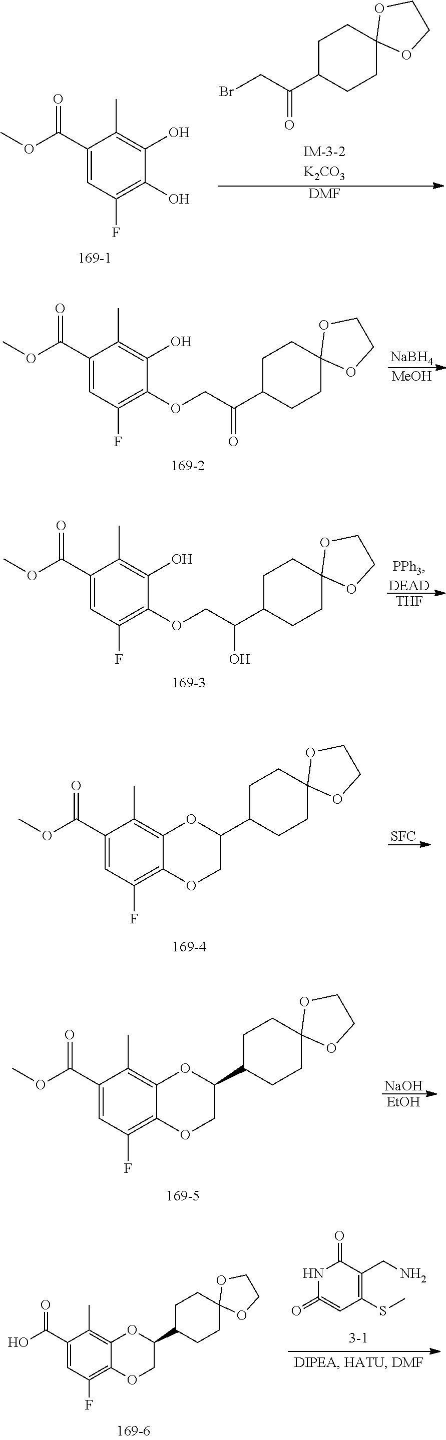

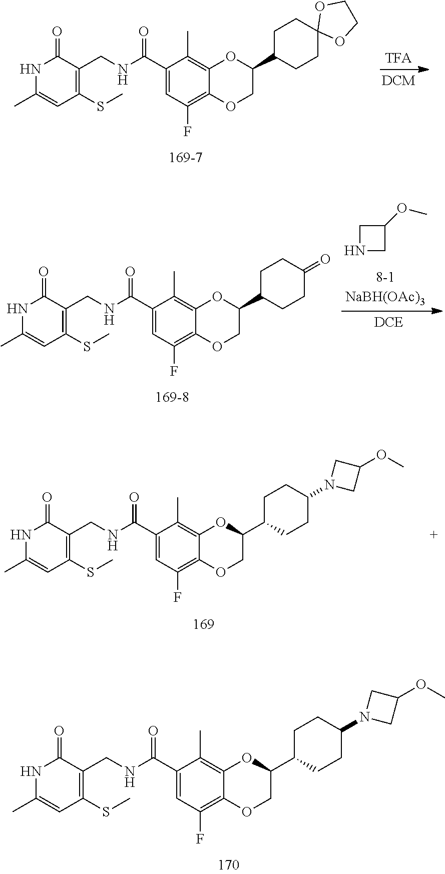

### Step 1, Compound 169-2 Synthesis

Following the synthesis method of Example 2, Step 1, except replacing Step 1, IM-2 with IM-3-2 (2.0 g, 7.6 mmol), 2-1 with 169-1 (1.0 g, 5.0 mmol), the same synthesis method was applied, to give Compound 169-2 (875.0 mg, crude). LCMS (ESI⁺) m/z: 383.1 [M+H]⁺

### Step 2, Compound 169-3 Synthesis

Following the synthesis method of Example IM-3, Step 3, except replacing Step 3, IM-3-4 with 169-2 (875.0 mg, crude), the same synthesis method was applied, to give Compound 169-3 (840.0 mg, crude). LCMS (ESI⁺) m/z: 385.2 [M+H]⁺

### Step 3, Compound 169-4 Synthesis

Following the synthesis method of Example IM-3, Step 4, except replacing Step 4, IM-3-5 with 169-3 (840.0 mg, crude), the same synthesis method was applied, to give Compound 169-4 (200.0 mg, 0.6 mmol, 25.0% yield). LCMS (ESI⁺) m/z: 367.2 [M+H]⁺

### Step 4, Compound 169-5 Synthesis

Compound 169-5 can be Obtained by SFC Resolution from Compound 169-4

### Step 5, Compound 169-6 Synthesis

Following the synthesis method of Example 6, Step 1, except replacing Step 1, IM-3a with 169-5 (86.0 mg, 0.2 mmol), the same synthesis method was applied, to give Compound 169-6 (101.0 mg, crude), LCMS (ESI⁺) m/z: 353.1 [M+H]⁺.

### Step 6, Compound 169-7 Synthesis

Following the synthesis method of Example 1, Step 3, except replacing Step 3, 1-3 with 169-6 (101.0 mg, crude), 1-4 with 3-1 (105.0 mg, 0.6 mmol), the same synthesis method was applied, to give Compound 169-7 (158.0 mg, crude), LCMS (ESI⁺) m/z: 519.9 [M+H]⁺.

### Step 7, Compound 169-8 Synthesis

Following the synthesis method of Example 3, Step 2, except replacing Step 2, 3-2 with 169-7 (158.0 mg, crude), the same synthesis method was applied, to give Compound 169-8 (132.0 mg, crude), LCMS (ESI⁺) m/z: 475.2 [M+H]⁺.

### Step 8, Compound 169 and 170 Synthesis

Following the synthesis method of Example 6, Step 4, except replacing Step 4, 6-3 with 169-8 (132.0 mg, crude), dimethylamine with 8-1 (48.0 mg, 0.6 mmol), the same synthesis method was applied. The obtained compound was subjected to Prep-MPLC to give 169 (26.0 mg, 0.05 mmol, 17.1% yield) and 170 (60.0 mg, 0.1 mmol, 39.5% yield), LCMS (ESI⁺) m/z: 546.6 [M+H]⁺.

Compound 169 NMR: ¹H NMR (600 MHz, DMSO-d₆) δ 11.51 (s, 1H), 7.95 (t, J=4.8 Hz, 1H), 6.73-6.71 (m, 1H), 6.08 (s, 1H), 4.41-4.39 (m, 1H), 4.27 (d, J=4.8 Hz, 2H), 4.03-4.00 (m, 1H), 3.95-3.88 (m, 2H), 3.43-3.41 (m, 2H), 3.13 (s, 3H), 2.75-2.70 (m, 2H), 2.45 (s, 3H), 2.16 (s, 3H), 2.11 (s, 3H) 1.96-1.85 (m, 2H), 1.80-1.69 (m, 3H), 1.58-1.52 (m, 1H), 1.19-1.15 (m, 2H), 0.91-0.84 (m, 2H).

¹⁹F NMR (564 MHz, DMSO-d₆) δ −140.00.

Compound 170 NMR: ¹H NMR (600 MHz, DMSO-d₆) δ 11.51 (s, 1H), 7.95 (t, J=4.2 Hz, 1H), 6.71 (d, J=10.8 Hz, 1H), 6.08 (s, 1H), 4.39-4.37 (m, 1H), 4.27 (d, J=4.2 Hz, 2H), 4.06-4.01 (m, 1H), 3.98-3.91 (m, 2H), 3.47-3.45 (m, 1H), 3.14 (s, 3H), 2.68-2.66 (m, 2H), 2.45 (s, 3H), 2.16 (s, 3H), 2.10 (s, 3H), 1.65-1.28 (m, 10H).

¹⁹F NMR (564 MHz, DMSO-d₆) δ −140.03.

### Example 99, Compound 171 and 172 Synthesis

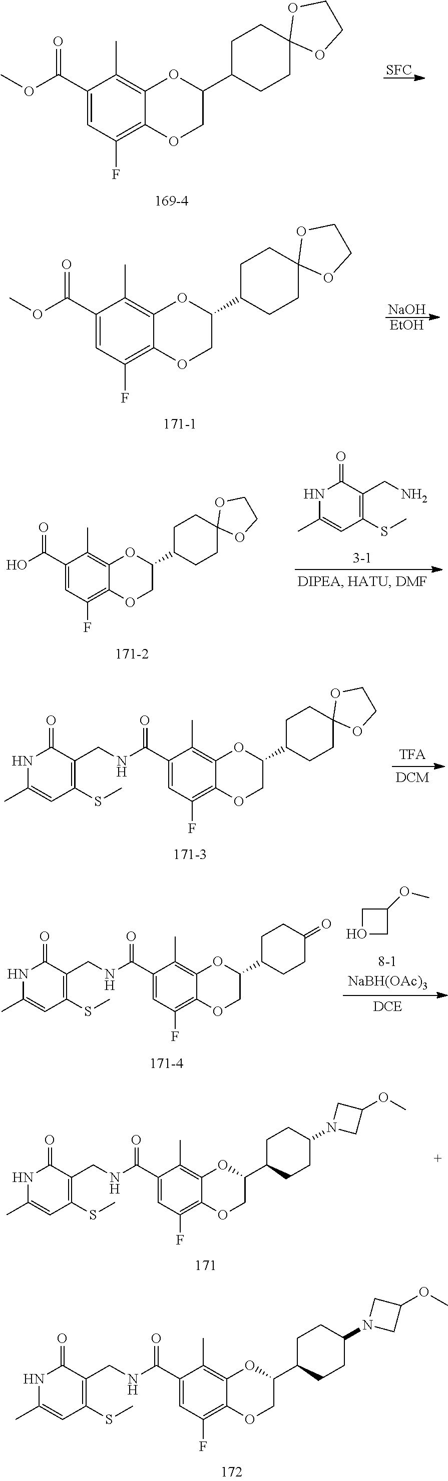

### Step 1, Compound 171-1 Synthesis

Compound 171-1 can be obtained by SFC resolution from Compound 169-4.

### Step 2, Compound 171-2 Synthesis

Following the synthesis method of Example 6, Step 1, except replacing Step 1, IM-3a with 171-1 (86.0 mg, 0.2 mmol), the same synthesis method was applied, to give Compound 171-2 (87.6 mg, crude), LCMS (ESI⁺) m/z: 353.1 [M+H]⁺.

### Step 3, Compound 171-3 Synthesis

Following the synthesis method of Example 1, Step 3, except replacing Step 3, 1-3 with 171-2 (87.6 mg, crude), 1-4 with 3-1 (92.0 mg, 0.5 mmol), the same synthesis method was applied, to give Compound 171-3 (155.0 mg, crude), LCMS (ESI⁺) m/z: 519.9 [M+H]⁺.

### Step 4, Compound 171-4 Synthesis

Following the synthesis method of Example 3, Step 2, except replacing Step 2, 3-2 with 171-3 (155.0 mg, crude), the same synthesis method was applied, to give Compound 171-4 (130.0 mg, crude), LCMS (ESI⁺) m/z: 475.2 [M+H]⁺.

### Step 5, Compound 171 and 172 Synthesis

Following the synthesis method of Example 6, Step 4, except replacing Step 4, 6-3 with 171-4 (130.0 mg, crude), dimethylamine with 8-1 (48.0 mg, 0.6 mmol), the same synthesis method was applied. The obtained compound was subjected to Prep-MPLC to give 171 (14.0 mg, 25.7 mol, 9.4% yield) and 172 (45.0 mg, 82.5 mol, 30.1% yield), LCMS (ESI⁺) m/z: 546.4 [M+H]⁺.

Compound 171 NMR: ¹H NMR (600 MHz, DMSO-d₆) δ 11.51 (s, 1H), 7.95 (t, J=4.2 Hz, 1H), 6.71 (d, J=10.8 Hz, 1H), 6.08 (s, 1H), 4.41-4.39 (m, 1H), 4.27 (d, J=4.2 Hz, 2H), 4.06-4.01 (m, 1H), 3.95-3.91 (m, 1H), 3.91-3.86 (m, 1H), 3.44-3.42 (m, 2H), 2.46-2.44 (m, 3H), 2.75-2.70 (m, 2H), 2.45 (s, 3H), 2.16 (s, 3H), 2.11 (s, 3H), 1.98-1.95 (m, 2H), 1.78-1.70 (m, 3H), 1.58-1.52 (m, 1H), 1.20-1.12 (m, 2H), 0.91-0.82 (m, 2H).

¹⁹F NMR (564 MHz, DMSO-d₆) δ −139.76.

Compound 172 NMR: ¹H NMR (600 MHz, DMSO-d₆) δ 11.51 (s, 1H), 7.95 (t, J=4.2 Hz, 1H), 6.71 (d, J=11.4 Hz, 1H), 6.08 (s, 1H), 4.39-4.34 (m, 1H), 4.27 (d, J=4.2 Hz, 2H), 4.06-4.01 (m, 1H), 3.99-3.90 (m, 2H), 3.50-3.42 (m, 2H), 3.14 (s, 3H), 2.70-2.64 (m, 2H), 2.45 (s, 3H), 2.16 (s, 3H), 2.11 (s, 3H), 1.65-1.26 (m, 10H).

¹⁹F NMR (564 MHz, DMSO-d₆) δ −140.03.

### Example 100, Compound 173 and 174 Synthesis

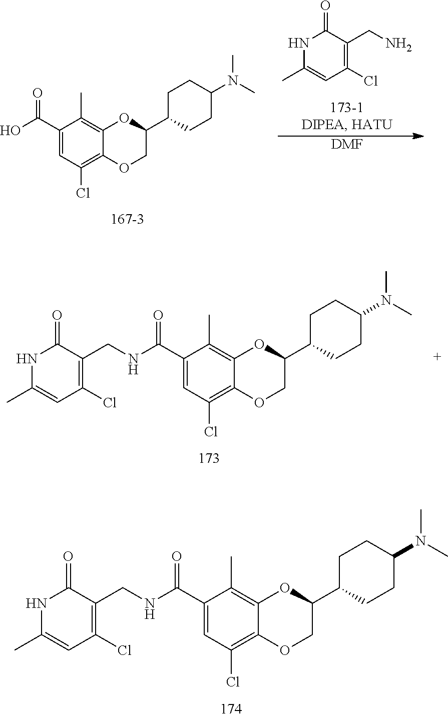

### Step 1, Compound 173 and 174 Synthesis

Following the synthesis method of Example 1, Step 3, except replacing Step 3, 1-3 with 167-3 (20.0 mg, 0.06 mmol), 1-4 with 173-1 (34.0 mg, 0.2 mmol), the same synthesis method was applied. The obtained compound was subjected to Prep-MPLC to give Compound 173 (1.0 mg, 2.0 mol, 3.5%) and Compound 174 (1.0 mg, 2.0 mol, 3.5%), LCMS (ESI⁺) m/z: 508.2 [M+H]⁺.

Compound 173 NMR: ¹H NMR (600 MHz, DMSO-d₆) δ 11.91 (s, 1H), 8.17 (t, J=4.8 Hz, 1H), 6.90 (s, 1H), 6.17 (s, 1H), 4.49 (d, J=9.6 Hz, 1H), 4.29 (d, J=4.8 Hz, 2H), 4.10-4.03 (m, 2H), 2.17-2.12 (m, 12H), 2.07-2.04 (m, 1H), 1.81-1.73 (m, 2H), 1.72-1.64 (m, 2H), 1.62-1.52 (m, 2H), 1.45-1.38 (m, 2H), 1.24-1.21 (m, 1H).

Compound 174 NMR: ¹H NMR (600 MHz, DMSO-d₆) δ 11.91 (s, 1H), 8.17 (t, J=4.8 Hz, 1H), 6.90 (s, 1H), 6.17 (s, 1H), 4.47 (d, J=10.2 Hz, 1H), 4.29 (d, J=4.2 Hz, 2H), 4.08-4.04 (m, 1H), 3.93-3.89 (m, 1H), 2.20-2.10 (m, 12H), 2.04-2.00 (m, 1H), 1.89-1.83 (m, 2H), 1.81-1.77 (m, 2H), 1.56-1.50 (m, 1H), 1.26-1.22 (m, 3H), 1.28-1.26 (m, 1H).

### Example 101, Compound 175 and 176 Synthesis

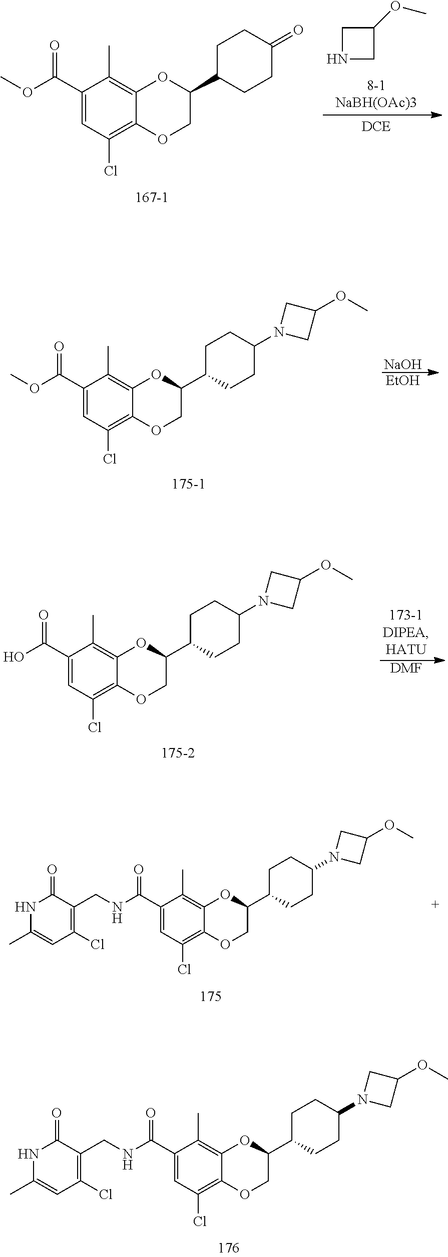

### Step 1, Compound 175-1 Synthesis

Following the synthesis method of Example 6, Step 4, except replacing Step 4, 6-3 with 167-1 (320.0 mg, 1.0 mmol), dimethylamine with 8-1 (85.0 mg, 1.9 mmol), the same synthesis method was applied, to give Compound 175-1 (275.0 mg, crude), LCMS (ESI⁺) m/z: 410.2 [M+H]⁺.

### Step 2, Compound 175-2 Synthesis

Following the synthesis method of Example 6, Step 1, except replacing Step 1, IM-3a with 175-1 (275.0 mg, crude), the same synthesis method was applied, to give Compound 175-2 (340.0 mg, crude), LCMS (ESI⁺) m/z: 396.2 [M+H]⁺.

### Step 3, Compound 175 and 176 Synthesis

Following the synthesis method of Example 1, Step 3, except replacing Step 3, 1-3 with 175-2 (20 mg, 0.05 mmol), 1-4 with 173-1 (17.0 mg, 0.1 mmol), the same synthesis method was applied.

The obtained compound was subjected to Prep-MPLC to give Compound 175 (3.5 mg, 6.4 mol, 12.6% yield) and Compound 176 (3.5 mg, 6.4 mol, 12.6% yield), LCMS (ESI⁺) m/z: 550.2 [M+H]⁺.

Compound 175 NMR: ¹H NMR (600 MHz, DMSO-d₆) δ 11.91 (s, 1H), 8.17 (t, J=4.8 Hz, 1H), 6.90 (s, 1H), 6.17 (s, 1H), 4.49 (d, J=9.6 Hz, 1H), 4.29 (d, J=4.8 Hz, 2H), 4.08-4.02 (m, 1H), 3.95-3.88 (m, 2H), 3.46-3.44 (m, 2H), 3.14 (s, 3H), 2.74-2.72 (m, 2H), 2.16 (s, 3H), 2.12 (s, 3H), 1.98-1.81 (m, 2H), 1.80-1.70 (m, 3H), 1.60-1.50 (m, 1H), 1.20-1.10 (m, 2H), 0.91-0.80 (m, 2H).

Compound 176 NMR: ¹H NMR (600 MHz, DMSO-d₆) δ 11.91 (s, 1H), 8.17 (t, J=4.8 Hz, 1H), 6.90 (s, 1H), 6.17 (s, 1H), 4.47 (dd, J=10.2, 2.0 Hz, 1H), 4.29 (d, J=4.8 Hz, 2H), 4.08-4.04 (m, 1H), 3.97-3.89 (m, 2H), 3.46-3.44 (m, 2H), 3.14 (s, 3H), 2.67 (t, J=6.0 Hz, 2H), 2.15 (s, 3H), 2.12 (s, 3H), 1.65-1.20 (m, 10H).

### Example 102, Compound 177 and 178 Synthesis

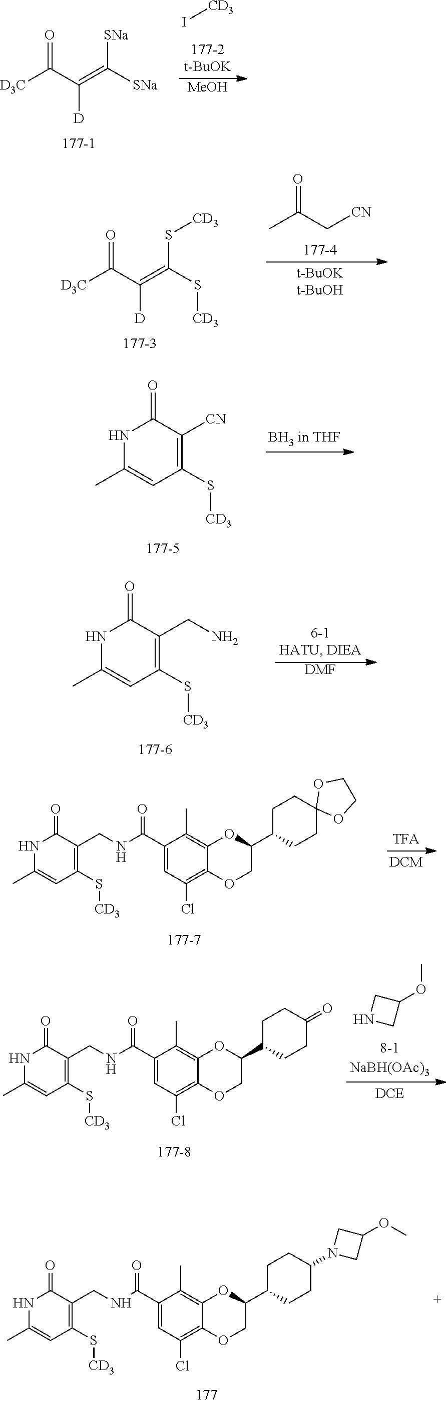

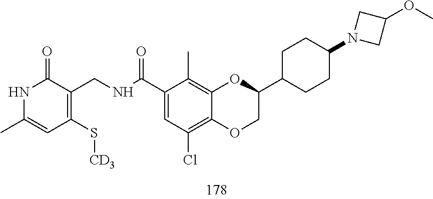

### Step 1, Compound 177-3 Synthesis

A single-necked flask was added with substrate 177-1 (100.0 mg, 0.5 mmol). The mixture was dissolved with methanol solution (5 mL) under stirring, and then added with 177-2 (80.0 mg, 0.5 mmol) and potassium t-butanolate (100.0 mg, 0.9 mmol), heated to 80° C. to react for 16 hours, with LC-MS monitoring. After the completion of the reaction, the reaction mixture was cooled to room temperature, added with water and ethyl acetate to extract three times. The organic phase was combined, washed with saturated saline solution, and dried with anhydrous sodium sulfate and then condensated under reduced pressure to give the crude product 177-3 (100.0 mg, crude), LCMS (ESI⁺) m/z: 172.3 [M+H]⁺.

### Step 2, Compound 177-5 Synthesis

A single-necked flask was added with substrate 177-3 (100.0 mg, crude). The mixture was dissolved with t-butanol solution (1 mL) under stirring, and then added with 177-4 (50.0 mg, 0.6 mmol) and potassium t-butanolate (78.0 mg, 0.7 mmol), heated to 80° C. to react for 1 hour, with LC-MS monitoring. After the completion of the reaction, the reaction mixture was cooled to room temperature, added with water and ethyl acetate to extract three times. The organic phase was combined, washed with saturated saline solution, and dried with anhydrous sodium sulfate and then condensated under reduced pressure to give the crude product 177-5 (90.0 mg, crude), LCMS (ESI⁺) m/z: 183.3 [M+H]⁺.

### Step 3, Compound 177-6 Synthesis

Under nitrogen protection, a three-necked flask was added with substrate 177-5 (90.0 mg, crude). The mixture was dissolved with borane solution in tetrahydrofuran (1 mL) under stirring. Then, heated to 80° C. to react for 5 hours, with LC-MS monitoring. After the completion of the reaction, the reaction mixture was cooled to room temperature, added with water and ethyl acetate to extract three times. The organic phase was combined, washed with saturated saline solution, and dried with anhydrous sodium sulfate and then condensated under reduced pressure to give the crude product 177-6 (50.0 mg, crude), LCMS (ESI⁺) m/z: 187.3 [M+H]⁺.

### Step 4, Compound 177-7 Synthesis

Following the synthesis method of Example 1, Step 3, except replacing Step 3, 1-3 with 6-1 (50.0 mg, 0.1 mmol), 1-4 with 177-6 (50.0 mg, 0.3 mmol), the same synthesis method was applied, to give Compound 177-7 (34.0 mg, crude), LCMS (ESI⁺) m/z: 537.2 [M+H]⁺.

### Step 5, Compound 177-8 Synthesis

Following the synthesis method of Example 3, Step 2, except replacing Step 2, 3-2 with 177-7 (34.0 mg, crude), the same synthesis method was applied, to give Compound 177-8 (34.0 mg, crude), LCMS (ESI⁺) m/z: 493.3 [M+H]⁺.

### Step 6, Compound 177 and 178 Synthesis

Following the synthesis method of Example 6, Step 4, except replacing Step 4, 6-3 with 177-8 (34.0 mg, crude), dimethylamine with 8-1 (19.0 mg, 0.2 mmol), the same synthesis method was applied. The obtained compound was subjected to Prep-MPLC to give 177 (1.5 mg, 2.7 mol, 3.9% yield) and 178 (1.5 mg, 2.7 mol, 3.9% yield), LCMS (ESI⁺) m/z: 565.2 [M+H]⁺.

Compound 177 NMR: ¹H NMR (600 MHz, DMSO-d₆) δ 11.51 (s, 1H), 8.01 (t, J=4.2 Hz, 1H), 6.90 (s, 1H), 6.07 (s, 1H), 4.42 (d, J=9.6 Hz, 1H), 4.27 (d, J=4.2 Hz, 2H), 4.08-4.03 (m, 1H), 3.96-3.89 (m, 2H), 3.47-3.43 (m, 3H), 3.14 (s, 3H), 2.66 (t, J=4.0 Hz, 2H), 2.21 (m, 1H), 2.16 (s, 3H), 2.13 (s, 3H), 1.64-1.53 (m, 4H), 1.42-1.25 (m, 4H).

Compound 178 NMR: ¹H NMR (600 MHz, DMSO-d₆) δ 11.51 (s, 1H), 8.01 (t, J=4.2 Hz, 1H), 6.90 (s, 1H), 6.07 (s, 1H), 4.42 (d, J=9.6 Hz, 1H), 4.27 (d, J=4.2 Hz, 2H), 4.08-4.03 (m, 2H), 3.93-3.86 (m, 2H), 3.13 (s, 3H), 2.71 (t, J=6.6 Hz, 2H), 2.16 (s, 3H), 2.15 (s, 3H), 1.98-1.85 (m, 2H), 1.80-1.70 (m, 3H), 1.57-1.50 (m, 1H), 1.36-1.10 (m, 3H), 0.92-0.80 (m, 2H).

### Example 103, Compound 179 and 180 Synthesis

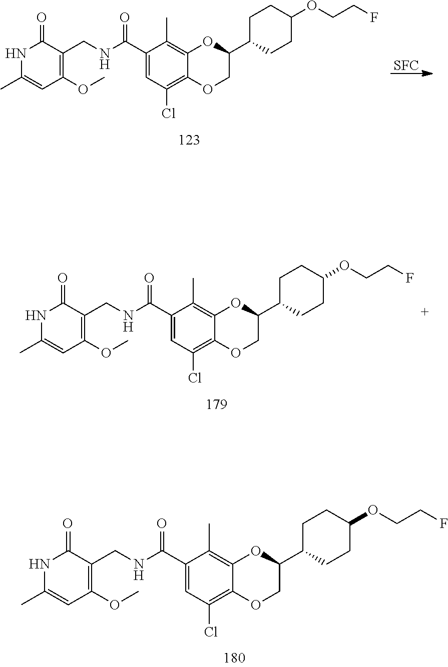

### Step 1, Compound 179 and 180 Synthesis

Compound 179 and 180 can be obtained by SFC resolution from Compound 123. LCMS (ESI⁺) m/z: 523.2 [M+H]⁺.

Compound 179 NMR: ¹H NMR (600 MHz, DMSO-d₆) δ 11.42 (s, 1H), 7.93 (t, J=4.2 Hz, 1H), 6.87 (s, 1H), 6.09 (s, 1H), 4.80-4.50 (m, 2H), 4.48-4.43 (m, 2H), 4.18 (d, J=4.8 Hz, 2H), 4.09-4.06 (m, 1H), 3.96-3.94 (m, 1H), 3.80 (s, 3H), 3.64-3.62 (m, 1H), 3.58-3.56 (m, 1H), 2.18 (s, 3H), 2.12 (s, 3H), 1.85-1.84 (m, 2H), 1.70-1.66 (m, 2H), 1.47-1.39 (m, 1H), 1.23-1.11 (m, 4H).

Compound 180 NMR: ¹H NMR (600 MHz, DMSO-d₆) δ 11.42 (s, 1H), 7.93 (t, J=4.8 Hz, 1H), 6.87 (s, 1H), 6.09 (s, 1H), 4.53-4.52 (m, 1H), 4.47-4.44 (m, 2H), 4.18 (d, J=4.8 Hz, 1H), 4.06-4.03 (m, 1H), 3.93-3.91 (m, 1H), 3.80 (s, 3H), 3.68-3.67 (m, 1H), 3.63-3.62 (m, 1H), 3.27-3.24 (m, 1H), 2.18 (s, 3H), 2.12 (s, 3H), 2.05-1.98 (m, 3H), 1.78-1.76 (m, 1H), 1.59-1.57 (m, 1H), 1.23-1.11 (m, 4H).

### Example 104, Compound 181 Synthesis

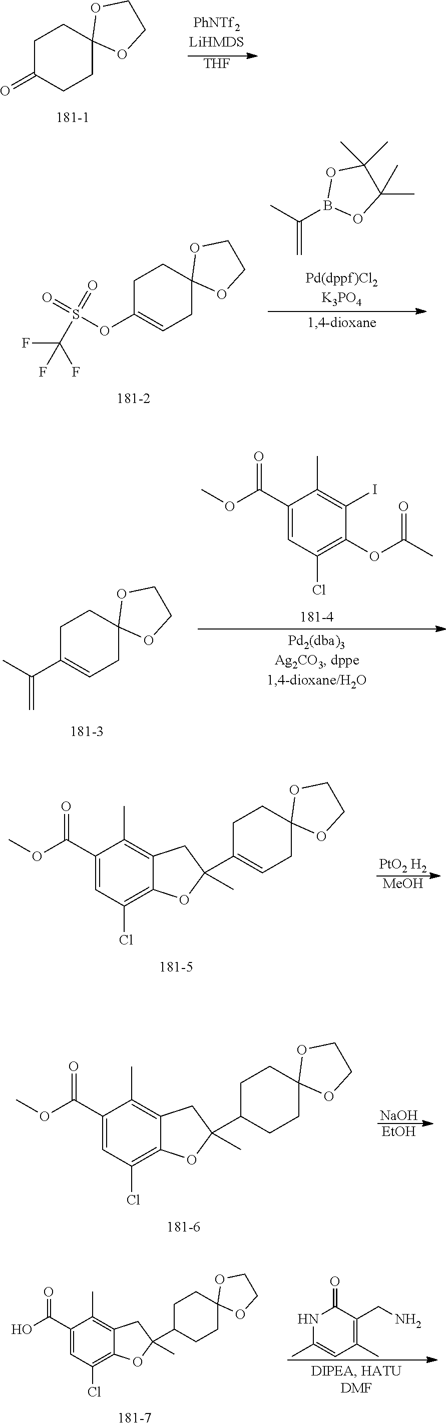

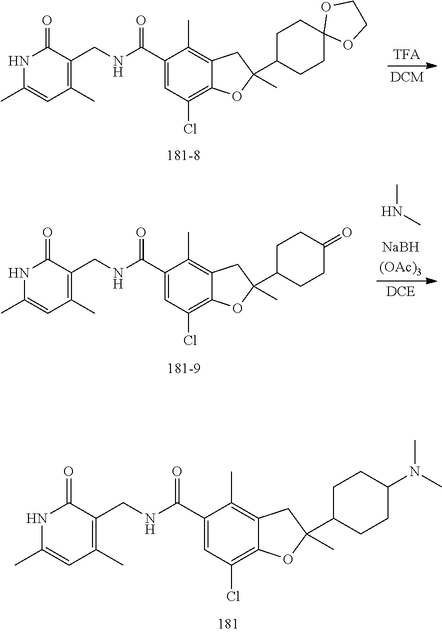

### Step 1, Compound 181-2 Synthesis

A single-necked flask was added with substrate 181-1 (3.0 g, 19.2 mmol). The mixture was dissolved with THF solution (50 mL) under stirring, and then at 0° C. added dropwise with LiHMDS (4.1 g, 25.0 mmol), at room temperature stirred for 0.5 hours, added with PhNTf₂ (8.9 g, 25.0 mmol), with LC-MS monitoring. After the completion of the reaction, the mixture was added with water and ethyl acetate to extract three times. The organic phase was combined, washed with saturated saline solution, and dried with anhydrous sodium sulfate, and then purified by silica gel column chromatography to give the product 181-2 (5.4 g, 18.7 mmol, 97.5% yield), LCMS (ESI⁺) m/z: 288.2 [M+H]⁺.

### Step 2, Compound 181-3 Synthesis

A single-necked flask was added with substrate 181-2 (2.9 g, 10.1 mmol). The mixture was dissolved with 1,4-dioxane solution (40 mL) under stirring, and then added with 2-isopropenyl-4,4,5,5-tetramethyl-1,3,2-dioxaborolane (2.0 g, 12.0 mmol), Pd(dppf)Cl₂ (817.0 mg, 1.0 mmol) and K₃PO₄ (3.2 g, 15.0 mmol), stirred at 80° C. for 18 hours, with LC-MS monitoring. After the completion of the reaction, the mixture was added with water and ethyl acetate to extract three times. The organic phase was combined, washed with saturated saline solution, and dried with anhydrous sodium sulfate, and then purified by silica gel column chromatography to give the product 181-3 (350.0 mg, 1.9 mmol, 19.2% yield), LCMS (ESI⁺) m/z: 180.2 [M+H]⁺.

### Step 3, Compound 181-5 Synthesis

A single-necked flask was added with substrate 181-3 (180.0 mg, 1.0 mmol). The mixture was dissolved with 1,4-dioxane solution (4 mL) and water (1 mL) under stirring, and then added with 181-4 (92.0 mg, 0.3 mmol), Pd₂(dba)₃ (11.4 mg, 0.01 mmol) and Ag₂CO₃ (138.0 mg, 0.5 mmol) and dppe (5.0 mg, 0.01 mmol), stirred at 100° C. for 24 hours, with LC-MS monitoring. After the completion of the reaction, the mixture was added with water and ethyl acetate to extract three times. The organic phase was combined, washed with saturated saline solution, and dried with anhydrous sodium sulfate, and then purified by silica gel column chromatography to give the product 181-5 (46.0 mg, 0.1 mmol, 12.1% yield), LCMS (ESI⁺) m/z: 378.2 [M+H]⁺.

### Step 4, Compound 181-6 Synthesis

A single-necked flask was added with substrate 181-5 (46.0 mg, 0.1 mmol), PtO₂ (5.0 mg, 0.02 mmol), and MeOH (3 mL). The system was reacted under 1 atm hydrogen atmosphere for 3 hours, to give Compound 181-6 (13.0 mg, 0.03 mmol, 28.1% yield), LCMS (ESI⁺) m/z: 380.9 [M+H]⁺.

### Step 5, Compound 181-7 Synthesis

Following the synthesis method of Example 6, Step 1, except replacing Step 1, IM-3a with 181-6 (13.0 mg, 0.03 mmol), the same synthesis method was applied, to give Compound 181-7 (12.0 mg, crude), LCMS (ESI⁺) m/z: 366.8 [M+H]⁺.

### Step 6, Compound 181-8 Synthesis

Following the synthesis method of Example 1, Step 3, except replacing Step 3, 1-3 with 181-7 (10.0 mg, 0.03 mmol), 1-4 with 2-4 (8.0 mg, 0.05 mmol), the same synthesis method was applied, to give Compound 181-8 (12.0 mg, 0.02 mmol, 87.6% yield), LCMS (ESI⁺) m/z: 501.0 [M+H]⁺.

### Step 7, Compound 181-9 Synthesis

Following the synthesis method of Example 3, Step 2, except replacing Step 2, 3-2 with 181-8 (12.0 mg, 0.02 mmol), the same synthesis method was applied, to give Compound 181-9 (8.0 mg, crude), LCMS (ESI⁺) m/z: 456.9 [M+H]⁺.

### Step 8, Compound 181 Synthesis

Following the synthesis method of Example 6, Step 4, except replacing Step 4, 6-3 with 181-9 (8.0 mg, 0.02 mmol), the same synthesis method was applied, to give Compound 181 (1.3 mg, 2.7 mol, 15.3% yield), LCMS (ESI⁺) m/z: 486.0 [M+H]⁺.

### Biological Evaluation Assay

### Test Example 1: Evaluation of the Inhibitory Effect of Compounds on EZH2 and EZH1 Proteins by the MTase-Glo Method

The compound was dissolved in DMSO, and the compound was serially diluted using ECHO665 and transferred to a 384-well reaction plate (Corning, 3824). A sample containing only DMSO was added as a positive control (maximum signal control) and a negative control (minimum signal control), while ensuring that the final DMSO content in each reaction well was 0.5%. The protein was configured in reaction buffer (50 mM Bicine, 0.01% Triton X-100, 1 mM DTT, 0.01% BSA, pH 7.6), such that the final reaction concentrations of EZH2/SUZ/EED (BPS, 51003) protein solution and EZH1/SUZ/EED (BPS, 51006) were 6.25 nM and 25 nM, respectively. The negative control (minimum signal control) used an equal amount of buffer instead of protein solution. The prepared protein solution was added to the 384-well reaction plate and pre-incubated at room temperature for 15 minutes. The mixture of the substrate H3 peptide fragment (custom-synthesized by GenScript, biotinylated Histone H3(21-44) peptide (ATKAARKSAPATGGVKKPHRYRP-GG-K(Biotin)-OH)) prepared in the reaction buffer and SAM (Promega, V7601) was added into the 384-well plate, such that the final reaction concentration of H3 peptide fragment was 10 μM, and the final concentration of SAM was 3 μM. The mixture was centrifuged at 1000 rpm for 1 min. The 384-well reaction plate was placed in a constant temperature shaker and incubate at 30° C. for 3 h at a speed of 300 rpm. Then the detection reagent was added according to the experimental process of the kit MTase-Glo (Promega, V7601) and incubated.

After incubation, read with BMG PHERAStar. The luminescence signals obtained by positive control (maximum signal control) and negative control (minimum signal control) were normalized to obtain the inhibition rate of different concentrations of compounds. Then, the IC₅₀ of the compound on the enzyme activity inhibition was calculated by GraphPad Prism 6 with log(inhibitor) vs. response-Variable slope model fitting. The fitting equation is: Y=Bottom+(Top−Bottom)/(1+10\hat{ }((Log IC₅₀−X)*HillSlope)), wherein Y represents the known percentage remaining enzyme activity, and X represents the known activity after Log compound concentration.

The results of Test Example 1 is shown in Table 1.

TABLE 1

| | Test Example 1 | Test Example 1 |
|---|---|---|
| | WT EZH2 | WT EZH1 |
| Compound | IC₅₀(nM) | IC₅₀(nM) |
| 1 | 27.5 | 1296 |
| 2 | 3.9 | 88.5 |
| 3 | 3.3 | 4.4 |
| 4 | 7208 | ND |
| 5 | 2.3 | 26 |
| 6 | 2.5 (mean, n = 3) | 4.0 (mean, n = 3) |
| 6′ | ND | ND |
| 7 | 130 | 1080 |
| 8 | 5.6 (mean, n = 2) | 6.2 |
| 9 | 3.1 | ND |
| 10 | 158 | ND |
| 11 | 8.1 (mean, n = 2) | 4.8 |
| 12 | 2.6 | ND |
| 13 | 17.7 | ND |
| 14 | 3.7(mean, n = 2) | 3.7 |
| 15 | 2.1 | ND |
| 16 | 5.2 | 29.6 |
| 17 | 4.6 | 16.5 |
| 18 | 3.5 | 7.4 |
| 19 | 3.4 | 26.3 |
| 20 | 3.1 | 3.3 |
| 21 | 6.4 | 18.9 |
| 22 | 2.1 | 2.3 |
| 23 | 2.4 | 8.1 |
| 24 | 4.5 | 9.2 |
| 25 | 1.6 | 8.3 |
| 26 | 2.0 | 5.4 |
| 27 | 2.3 | 14.4 |
| 28 | 3.7 | 9.4 |
| 29 | 3.3 | 5.8 |
| 30 | 3.3 | 17.9 |
| 31 | 66.3 | 491 |
| 32 | 5.1 | 5.7 |
| 33 | 3.6 | 9.4 |
| 34 | 4.1 | 6.0 |
| 35 | 3.4 | 7.4 |
| 36 | 2.4 | 9.7 |
| 37 | 3.1 | 5.9 |
| 38 | 2.3 | 4.7 |
| 39 | 4.0 | 7.3 |
| 40 | ND | 7.3 |
| 41 | 4.3 | 3.9 |
| 42 | ND | 8.6 |
| 43 | 3.6 | 4.1 |
| 44 | ND | 13.5 |
| 45 | 9.5 | 3.0 |
| 46 | ND | 7.7 |
| 47 | 1.2 | 9.5 |
| 48 | ND | 6.0 |
| 49 | ND | 2.2 |
| 50 | ND | 11.8 |
| 51 | ND | ND |
| 52 | ND | ND |
| 53 | 3.0 (mean, n = 3) | 26.8 (mean, n = 3) |
| 54 | 250 | 400 |
| 55 | 4.5 | 150 |
| 56 | 340 | 810 |
| 57 | 21.7 | 260 |
| 58 | 35.8 | 2297 |
| 59 | 9.6 | 7.0 |
| 60 | 16.4 | 94.9 |
| 61 | 8.8 | 162 |
| 62 | 22.1 | 1441 |
| 63 | 11.5 | 375 |
| 64 | 16.6 | 254 |
| 65 | 29.7 | 641 |
| 66 | 24.4 | 687 |
| 67 | 47.6 | 367 |
| 68 | 59.3 | >10000 |
| 69 | 84.6 | 879 |
| 70 | 22.1 | 177 |
| 71 | 41.8 | 564 |
| 72 | 1254 | >10000 |
| 73 | 51.3 | 196 |
| 74 | 998 | >10000 |
| 75 | 48.1 | 199 |
| 76 | 7.6 | 270 |
| 77 | 27.3 | 142 |
| 78 | 11.9 | ND |
| 79 | 2.0 | ND |
| 80 | 49.3 | ND |
| 81 | 31.0 | ND |
| 82 | 85.1 | ND |
| 83 | 8.2 | ND |
| 84 | 11.0 | ND |
| 85 | 10.5 | ND |
| 86 | 20.1 | ND |
| 87 | 780 | ND |
| 88 | 29.5 | ND |
| 89 | 1844 | ND |
| 90 | 18.6 (mean, n = 2) | 945 (mean, n = 2) |
| 91 | 3.8 | 27.3 |
| 92 | 2.5 | 33.1 |
| 93 | 6.0 | 132.4 |
| 94 | 3.0 (mean, n = 2) | 8.1 (mean, n = 3) |
| 95 | 11.6 | 227 |
| 96 | 7.0 | 167 |
| 97 | 3.2455 (mean, n = 2) | 15.22 |
| 98 | 5.7 | ND |
| 99 | 93.4 | ND |
| 100 | 127.7 | ND |
| 101 | 3.1 | 5.5 |
| 102 | 2.6 | 655 |
| 103 | ND | ND |
| 104 | ND | ND |
| 105 | ND | ND |
| 106 | ND | ND |
| 107 | 3.2 | 17.8 |
| 108 | 5.0 | 69.3 |
| 109 | 2.0 | 3.8 |
| 110 | 2.7 | 15.4 |
| 111 | 13.4 | 27.3 |
| 112 | ND | 49.9 |
| 113 | 9.4 | 91.2 |
| 114 | ND | 267 |
| 115 | 4.9 | 74.8 |
| 116 | ND | 156 |
| 117 | 6.2 | ND |
| 118 | ND | ND |
| 119 | ND | ND |
| 120 | ND | ND |
| 121 | ND | ND |
| 122 | ND | ND |
| 123 | ND | 84 |
| 124 | 16.1 | 326 |
| 125 | 33.9 | 307 |
| 126 | 20.3 (mean, n = 2) | 218 |
| 127 | 25.5 | ND |
| 128 | 9.5 | ND |
| 129 | 3.9 | ND |
| 130 | 12.6 | 201 |
| 131 | 3.1 | 46.8 |
| 132 | 7.3 | 722 |
| 133 | 16.6 | 584 |
| 134 | 6.6 | 125 |
| 135 | 80.1 | 5605 |
| 136 | 42.5 | 192 |
| 137 | ND | ND |
| 138 | ND | ND |
| 139 | 1256 | 8490 |
| 140 | 897 | 8914 |
| 141 | 179 | >10000 |
| 142 | 1163 | 7611 |
| 143 | 5.5 | 39.9 |
| 144 | 15.6 | 769 |
| 145 | ND | ND |
| 146 | ND | ND |
| 147 | 6.8 | 92.0 |
| 148 | 67.1 | 2825 |
| 149 | 2.3 | 3.3 |
| 150 | 11.6 | 22.2 |
| 151 | ND | ND |
| 152 | ND | 126 |
| 153 | 3.2 | 6.9 |
| 154 | 4.2 | 30.0 |
| 155 | 21.4 | 585 |
| 156 | 67.0 | 366 |
| 157 | ND | 190 |
| 158 | ND | 3435 |
| 159 | 1.1 | 7.0 |
| 160 | ND | 31.6 |
| 161 | 3.7 | 5.5 |
| 162 | ND | 29.3 |
| 163 | 7.1 | ND |
| 164 | ND | ND |
| 165 | 3.6 | 5.7 |
| 166 | ND | 17.13 |
| 167 | ND | ND |
| 168 | ND | ND |
| 169 | 6.0 | 5.8 |
| 170 | ND | ND |
| 171 | ND | ND |
| 172 | ND | ND |
| 173 | ND | ND |
| 174 | 3.1 | 7.2 |
| 175 | 3.4 | 16.6 |
| 176 | ND | ND |
| 177 | 1.5 | 2.3 |
| 178 | ND | 17.5 |
| 179 | ND | ND |
| 180 | ND | ND |
| 181 | 30.4 | 1347 |

### Test Example 2: Evaluation of the Effect of Compounds on Karpas-422 Cells in Inhibiting H3K27Me3 Methylation by H3K27Me3 Cell Method

Karpas-422 (Nanjing Kebai, CBP60629) cells were seeded in a 96-well plate (Corning, 3894) at 15,000 cells per well in culture medium (80% 1640 (Hyclone, SH30809.01)+20% FBS (Corning, 35-081-CV)), and then added with different concentrations of compounds for treatment. The cell growth group without adding compounds is used as a positive control (maximum signal control), and the medium is used as a negative control (minimum signal control), while ensuring that in each reaction well, the final concentration of DMSO was 0.2%. Compounds and cells were incubated for 3 days in a cell culture incubator (37° C., 5% CO₂). The 96-well plate was taken out from the cell culture incubator and equilibrated to room temperature, and 25 ul of cell suspension was taken out into a 384-well plate. The CCK8 (Beiyuntian, C0040) detection reagent was added, incubated at 37° C. for 1 h, and OD405 nm was detected. The rest of the cells followed the procedure of the kit EPIgeneous H₃K₂₇Me₃ cellular kit (Cisbio, 62KC3PAE) to carry out experiment, read HTRF signal with BMG PHERAStar. The inhibition rate was calculated according to HTRF signal: first calculating positive control (maximum signal control) and negative control (minimum signal control) mean value, with inhibition rate=[1−(compound signal value−negative control signal value)/(positive control signal value-negative control signal value)]×100% to calculate the inhibition rate of different concentrations of compound cells. By GraphPad Prism 6 with log(inhibitor)vs.response-Variable slope model simulation, the IC₅₀ of the compound on cell activity inhibition was calculated. The fitting equation is: Y=Bottom+(Top−Bottom)/(1+10\hat{ }((Log IC₅₀−X)*HillSlope)), wherein Y represents the inhibition rate, and X represents the known concentration of the compound after Log.

### Test Example 3: Evaluation of the Anti-Proliferation Effect of Compounds on Karpas-422 Cells by Cell Titer-Glo Method

Karpas-422 (Nanjing Kebai, CBP60629) cells were seeded in a 96-well plate (Corning, 3894) at 1,000 cells per well in growth medium (80% 1640 (Hyclone, SH30809.01)+20% FBS (Corning, 35-081-CV)), in a total of 100 μL, and then was added with 25 μL of 5-fold different concentrations of compounds diluted in growth medium. The cell growth group without compound addition was used as a positive control (maximum signal control), and the medium was used as a negative control (minimum signal control), while ensuring that the final contration of DMSO in each reaction well was 0.2%. Then the 96-well cell plate was placed in a cell culture incubator (37° C., 5% CO₂) and incubated for 3 days. On the fourth day, the cell culture plate was taken out of the incubator, using a row gun to gently blow the cells up and down to mix, removing 62.5 μL of the cell suspension, adding 50 μL of growth medium and 12.5 μL of compounds of 5 times different concentrations, and then putting them in the cell incubator to incubate for 4 days. On the eighth day, the same method was used as the fourth day to treat the cells, then incubating in the cell culture incubator for 3 days. On the eleventh day, the Cell-Titer Glo (Promega, G7573) was used to detect cell viability, and a microplate reader was used to detect the signal value of luminescence.

The inhibition rate of different concentrations of compounds on cells was calculated as inhibition rate=[1−(compound signal value−negative control signal value)/(positive control signal value-negative control signal value)]×1000. The IC₅₀ of the compound on cell activity inhibition was calculated by GraphPad Prism 6 with log(inhibitor) vs. response-Variable slope model fitting. The fitting equation is: Y=Bottom+(Top−Bottom)/(1+10\hat{ }((Log IC₅₀−X)*Hill Slope)), wherein Y represents the inhibition rate, and X represents the known concentration of the compound after Log.

The results of Test Example 2 and 3 are shown in Table 2

TABLE 2

| | Test Example 2 | Test Example 3 |
|---|---|---|
| | H₃K₂₇Me₃ inhibition | Karpas-422 cell |
| | (Karpas-422) | anti-proliferation |
| Compound | IC₅₀(nM) | IC₅₀(nM) |
| 1 | ND | ND |
| 2 | 31.2 (mean, n = 3) | 42.4 (mean, n = 2) |
| 3 | 2.1 | 7.6 (mean, n = 2) |
| 4 | ND | ND |
| 5 | 7.9 | 23.2 |
| 6 | 0.59 (mean, n = 2) | 1.3 (mean, n = 2) |
| 6′ | ND | ND |
| 7 | >1000 | >1000 |
| 8 | 2.4 (mean, n = 2) | 1.8 |
| 9 | 14.2 | ND |
| 10 | ND | ND |
| 11 | 1.7 | 1.5 |
| 12 | 9.4 | ND |
| 13 | ND | ND |
| 14 | 6.8 | 3.3 |
| 15 | 101 | ND |
| 16 | 85.9 | 19.8 |
| 17 | 12.0 | ND |
| 18 | 22.8 | 17.7 |
| 19 | 284 | 89.5 |
| 20 | 3.2 | 1.6 |
| 21 | ND | ND |
| 22 | 1.8 | ND |
| 23 | 22.8 | ND |
| 24 | 9.8 | ND |
| 25 | 17.3 | ND |
| 26 | 3.4 | ND |
| 27 | 11.4 | ND |
| 28 | 8.2 | ND |
| 29 | 34.2 | ND |
| 30 | 23.3 | ND |
| 31 | ND | ND |
| 32 | 1.7 | 2.0 |
| 33 | 7.7 | ND |
| 34 | 6.2 | 5.8 |
| 35 | 60.5 | ND |
| 36 | 11.0 | ND |
| 37 | 7.0 | ND |
| 38 | 11.5 | ND |
| 39 | ND | ND |
| 40 | 8.3 | ND |
| 41 | ND | ND |
| 42 | 47.1 | ND |
| 43 | 4.1 | ND |
| 44 | ND | ND |
| 45 | 6.6 | ND |
| 46 | 65.8 | ND |
| 47 | 8.9 | ND |
| 48 | 27.9 | ND |
| 49 | 2.8 | ND |
| 50 | 23.8 | ND |
| 51 | ND | ND |
| 52 | ND | ND |
| 53 | 5.0 (mean, n = 2) | 14.1 (mean, n = 2) |
| 54 | >1000 | >1000 |
| 55 | 19.4 | 47.3 |
| 56 | 944 | >1000 |
| 57 | >1000 | ND |
| 58 | >1000 | ND |
| 59 | 233 | ND |
| 60 | >1000 | ND |
| 61 | 73.8 (mean, n = 2) | 93.8 |
| 62 | 504 | ND |
| 63 | 616 | 341 |
| 64 | 896 | ND |
| 65 | ND | ND |
| 66 | ND | ND |
| 67 | ND | ND |
| 68 | ND | ND |
| 69 | ND | ND |
| 70 | 201 | ND |
| 71 | ND | ND |
| 72 | ND | ND |
| 73 | >1000 | ND |
| 74 | ND | ND |
| 75 | 245 | ND |
| 76 | 7.6 | ND |
| 77 | 260 | ND |
| 78 | 370 | ND |
| 79 | ND | ND |
| 80 | ND | ND |
| 81 | >1000 | ND |
| 82 | ND | ND |
| 83 | ND | ND |
| 84 | ND | ND |
| 85 | 95.5 | ND |
| 86 | >1000 | ND |
| 87 | ND | ND |
| 88 | 310 | ND |
| 89 | ND | ND |
| 90 | ND | ND |
| 91 | 11.7 | ND |
| 92 | 22.0 | ND |
| 93 | ND | ND |
| 94 | 2.8 (mean, n = 2) | 5.1 |
| 95 | ND | ND |
| 96 | ND | ND |
| 97 | 8.2 (mean, n = 2) | 5.3 (mean, n = 2) |
| 98 | 462 | ND |
| 99 | ND | ND |
| 100 | ND | ND |
| 101 | 2.2 | 3.8 |
| 102 | ND | ND |
| 103 | ND | ND |
| 104 | ND | ND |
| 105 | ND | ND |
| 106 | ND | ND |
| 107 | 56.9 | ND |
| 108 | ND | ND |
| 109 | 1.8 | 2.5 |
| 110 | 21.3 | ND |
| 111 | ND | ND |
| 112 | ND | ND |
| 113 | ND | ND |
| 114 | ND | ND |
| 115 | ND | ND |
| 116 | ND | ND |
| 117 | 39.4 | 26.1 (mean, n = 2) |
| 118 | ND | 209 |
| 119 | ND | 12.9 |
| 120 | ND | 31.5 |
| 121 | 92.3 | 35.8 |
| 122 | ND | 472 |
| 123 | ND | ND |
| 124 | 133 | ND |
| 125 | 402 | ND |
| 126 | 818 | ND |
| 127 | ND | ND |
| 128 | 43.2 | ND |
| 129 | ND | ND |
| 130 | 209 | 115 |
| 131 | 43.3 | ND |
| 132 | ND | ND |
| 133 | ND | ND |
| 134 | ND | ND |
| 135 | ND | ND |
| 136 | ND | ND |
| 137 | ND | ND |
| 138 | ND | ND |
| 139 | ND | ND |
| 140 | ND | ND |
| 141 | ND | ND |
| 142 | ND | ND |
| 143 | 22.0 | 10.7 |
| 144 | ND | ND |
| 145 | ND | ND |
| 146 | ND | ND |
| 147 | 83.0 | ND |
| 148 | ND | ND |
| 149 | 3.6 | 2.7 |
| 150 | ND | ND |
| 151 | 13.2 | ND |
| 152 | ND | ND |
| 153 | 6.2 | 3.7 |
| 154 | ND | ND |
| 155 | ND | ND |
| 156 | ND | ND |
| 157 | ND | ND |
| 158 | ND | ND |
| 159 | 1.4 | 2.3 |
| 160 | ND | ND |
| 161 | 2.2 | 2.4 |
| 162 | ND | ND |
| 163 | 1.9 | 1.7 (mean, n = 2) |
| 164 | ND | ND |
| 165 | 1.4 | 1.2 (mean, n = 2) |
| 166 | ND | ND |
| 167 | ND | >1000 |
| 168 | ND | >1000 |
| 169 | 4.2 | 1.8 |
| 170 | ND | 46.8 |
| 171 | >1000 | >1000 |
| 172 | ND | 373 |
| 173 | ND | 124 |
| 174 | ND | 5.6 |
| 175 | 16.1 | 13.6 |
| 176 | ND | 197 |
| 177 | ND | ND |
| 178 | ND | ND |
| 179 | ND | 36.6 |
| 180 | ND | 10.8 |
| 181 | 410 | ND |

### Test Example 4: Karpas-422 Subcutaneous Transplantation Model Evaluation of Antitumor Activity Against EZH2 Y641N Mutant Human Diffuse Large B-Cell Lymphoma

### 1. Experimental Materials

Karpas-422 are human diffuse large B-cell lymphoma cells. NCG mice, female, 7 weeks old, weighing 19-22 grams, were purchased from Chengdu Yaokang Biotechnology Co., Ltd.

### 2. Experimental Method

Karpas-422 cells in logarithmic growth period were collected, counted, and the final concentration of cells was adjusted. The matrix glue of Matrigel, which was completely liquefied, was mixed at a ratio of 1:1. 1×10⁷ cells/mouse were inoculated under the right armpit, and the inoculation volume was 100 μL/mouse. When the average tumor volume reached about 188 mm³, they were randomly divided into groups (n=7). The solvent used in Compound (DS-3201, Compound 8 and Compound 97) was DMSO: Solutol: TIPBCD (20%, w/v)=5:20:75. From the day of grouping, Compound of Example A, Example B or Example C were administered orally, the dose was set at 72 or 144 mg/kg/day, and the dosage regimen was 2 times a day for 28 consecutive days (BID×28). During the experiment, animal activities were observed once a day, each animal was weighed once before administration, and the long diameter and short diameter of the tumor were measured with a vernier caliper twice a week. After the experiment was over, all surviving experimental animals were sacrificed.

### 3. Data Analysis

The calculation formula of the tumor volume is: V=0.5(a×b²), where a and b represent the long diameter and short diameter of the tumor respectively;

$$Tumor growth inhibition degree TGI ( \% ) = ( 1 - TVCt / TVCc ) \times 100 ;$$

TVC=(Tumor volume per individual on the end of test day)-(Tumor volume per individual on grouping day);
TVCt: the average TVC of the administration group; TVCc: the average TVC of the non-administration group.

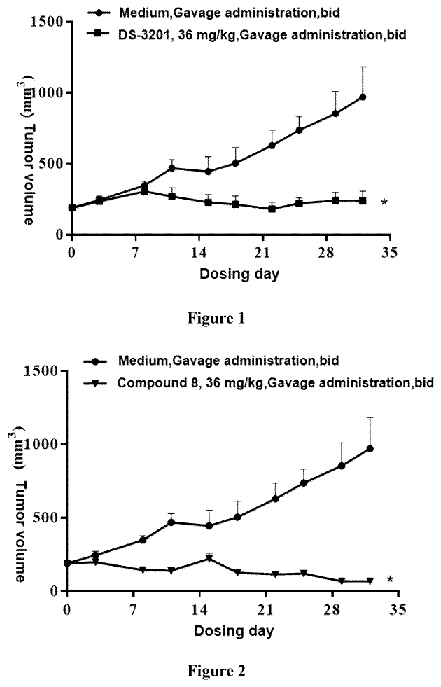

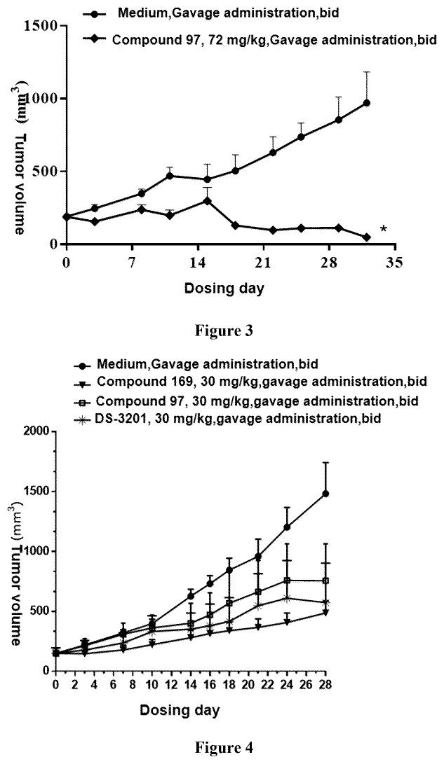

The results are shown in FIGS. 1-3 of the drawings in the description.

The compounds of the present invention are significantly superior to other compounds in their inhibitory activity against EZH2 and EZH1 kinases, Karpas 422 cell inhibitory activity and antiproliferative activity against Karpas 422 cells.

### Test Example 5: X1LNCaP Subcutaneous Transplantation Model Evaluation of Prostate Cancer Antitumor Activity

### 1. Experimental Materials

LNCaP is a human prostate cancer cell line. The X1LNCaP cell line is derived from the LNCaP cell line: LNCaP cells were inoculated into mice, xenograft tumors of about 300-500 mm³ were removed from the mice, and tumor cells were isolated and cultured. These recultured cells were inoculated into mice, and the model was named X1LNCaP xenograft model. BALB/c nude mice, male, 6-8 weeks old, weighing 18-20 g, were purchased from LC.

### 2. Experimental Method

A 0.2 mL cell suspension containing 1×10⁷ cells (cell suspension in RPMI-1640: Matrigel=100 μL: 100 μL) was subcutaneously inoculated on the right back of the mouse. When the tumor volume reached about 100-150 mm³, they were randomized (n=3). The solvent used for the compounds (DS-3201, Compound 97 and Compound 169) was DMSO:Solutol:HPBCD (20%, W/V)=5:20:75. From the day of grouping, DS-3201, Compound 97 or Compound 169 were administered orally, the dose was set at 60 mg/kg/day, and the dosage regimen was 2 times a day for 28 consecutive days (BID×28). During the experiment, animal activities were observed once a day, each animal was weighed once before administration, and the long diameter and short diameter of the tumor were measured with a vernier caliper twice a week. After the experiment was over, all surviving experimental animals were sacrificed.

### 3. Data Analysis

The calculation formula of the tumor volume is: V=0.5(a×b²), where a and b represent the long diameter and short diameter of the tumor respectively;

$$Tumor growth inhibition degree TGI ( \% ) = ( 1 - ( {TV}_{Treatment / Dx} - {TV}_{Treatment / D 1} ) / ( {TV}_{Control / Dx} - {TV}_{Control / D 1} ) \times 100 \% ;$$

$$the results are shown in Figure 4.$$

1. A compound represented by Formula I or Formula II, or a deuterated compound thereof, or a stereoisomer thereof, or a pharmaceutically acceptable salt thereof: 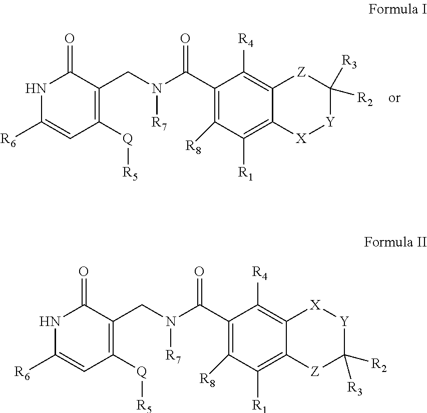
wherein,
X is selected from a group consisting of single bond, O, NR^{X1} or CHR^{X2};
Y is selected from a group consisting of O, S, NR^{Y1} or CHR^{Y2};
Z is selected from a group consisting of C═O, O, S, NR^{Z1} or CHR^{Z2};
Q is selected from a group consisting of single bond, O or S;
when X is single bond, Y and Z are not both O;
R^{X1} is selected from a group consisting of the following groups: hydrogen, —C_{1˜6}alkyl, halo-C_{1˜6}alkyl, —C_{0˜2}alkylene-(3˜10 membered cycloalkyl), —C_{0˜2}alkylene-(3˜10 membered heterocycloalkyl), —C_{0˜2}alkylene-(5˜10 membered aryl ring), —C_{0˜2}alkylene-(5˜10 membered heteroaryl ring);
R^{X2} is selected from a group consisting of the following groups: hydrogen, halogen, cyano, hydroxyl, —C_{1˜6}alkyl, halo-C_{1˜6}alkyl, —O(C_{1˜6}alkyl), —O(halo-C_{1˜6}alkyl), —NH₂, —NH(C_{1˜6}alkyl), —N(C_{1˜6}alkyl) (C_{1˜6}alkyl), —C_{0˜2}alkylene-(3˜10 membered cycloalkyl), —C_{0˜2}alkylene-(3˜10 membered heterocycloalkyl), —C_{0˜2}alkylene-(5˜10 membered aryl ring), —C_{0˜2}alkylene-(5˜10 membered heteroaryl ring);
R^{Y1} is selected from a group consisting of the following groups: hydrogen, —C_{1˜6}alkyl, halo-C_{1˜6}alkyl, —C_{0˜2}alkylene-(3˜10 membered cycloalkyl), —C_{0˜2}alkylene-(3˜10 membered heterocycloalkyl), —C_{0˜2}alkylene-(5˜10 membered aryl ring), —C_{0˜2}alkylene-(5˜10 membered heteroaryl ring);
R^{Y2} is selected from a group consisting of the following groups: hydrogen, halogen, cyano, hydroxyl, —C_{1˜6}alkyl, halo-C_{1˜6}alkyl, —O(C_{1˜6}alkyl), —O(halo-C_{1˜6}alkyl), —NH₂, —NH(C_{1˜6}alkyl), —N(C_{1˜6}alkyl) (C_{1˜6}alkyl), —C_{0˜2}alkylene-(3˜10 membered cycloalkyl), —C_{0˜2}alkylene-(3˜10 membered heterocycloalkyl), —C_{0˜2}alkylene-(5˜10 membered aryl ring), —C_{0˜2}alkylene-(5˜10 membered heteroaryl ring);
R^{Z1} is selected from a group consisting of the following groups: hydrogen, C_{1˜6}alkyl, halo-C_{1˜6}alkyl, —C_{0˜2}alkylene-(3˜10 membered cycloalkyl), —C_{0˜2}alkylene-(3˜10 membered heterocycloalkyl), —C_{0˜2}alkylene-(5˜10 membered aryl ring), —C_{0˜2}alkylene-(5˜10 membered heteroaryl ring);
R^{Z2} is selected from a group consisting of the following groups: hydrogen, halogen, cyano, hydroxyl, —C_{1˜6}alkyl, halo-C_{1˜6}alkyl, —O(C_{1˜6}alkyl), —O(halo-C_{1˜6}alkyl), —NH₂, —NH(C_{1˜6}alkyl), —N(C_{1˜6}alkyl) (C_{1˜6}alkyl), —C_{0˜2}alkylene-(3˜10 membered cycloalkyl), —C_{0˜2}alkylene-(3˜10 membered heterocycloalkyl), —C_{0˜2}alkylene-(5˜10 membered aryl ring), —C_{0˜2}alkylene-(5˜10 membered heteroaryl ring);
R₁ is selected from a group consisting of the following groups: hydrogen, halogen, cyano, nitro, amino, hydroxyl, —C_{1˜6}alkyl, —O(C_{1˜6}alkyl), halo-C_{1˜6}alkyl, —C_{0˜2}alkylene-OR^{1b}, —C_{0˜2}alkylene-C(O)R^{1b}, —C_{0˜2}alkylene-C(O)NR^{1b}R^{1c}—C_{0˜2}alkylene-NR^{1b}R^{1c}—C_{0˜2}alkylene-NR^{1b}C(O)R^{1c}, —C_{0˜4}alkylene-S(O)₂R^{1b}, —C_{0˜2}alkylene-(3˜10 membered cycloalkyl), —C_{0˜2}alkylene-(3˜10 membered heterocycloalkyl), —C_{0˜2}alkylene-(5˜10 membered aryl ring), —C_{0˜2}alkylene-(5˜10 membered heteroaryl ring); wherein said alkyl, alkylene, cycloalkyl, heterocycloalkyl, aryl ring, heteroaryl ring is optionally further substituted by one, two or three independent R^{1b};
R^{1b}, R^{1c} is each independently selected from a group consisting of the following groups: hydrogen, —OH, C_{1˜6}alkyl, halo-C_{1˜6}alkyl, —O(C_{1˜6}alkyl), —O(halo-C_{1˜6}alkyl), —NH₂, —NH(C_{1˜6}alkyl), —N(C_{1˜6}alkyl) (C_{1˜6}alkyl), —C_{0˜2}alkylene-(3˜10 membered cycloalkyl), —C_{0˜2}alkylene-(3˜10 membered heterocycloalkyl), —C_{0˜2}alkylene-(5˜10 membered aryl ring), —C_{0˜2}alkylene-(5˜10 membered heteroaryl ring);
R₂ is selected from a group consisting of the following groups: —C_{0˜2}alkylene-(3˜10 membered cycloalkyl), —C_{0˜2}alkylene-(3˜10 membered heterocycloalkyl); wherein said alkylene, cycloalkyl, heterocycloalkyl is optionally further substituted by one, two or three independent R^{2b};
R^{2b} is each independently selected from a group consisting of the following groups: hydrogen, halogen, cyano, nitro, amino, hydroxyl, —C_{1˜6}alkyl, —O(C_{1˜6}alkyl), halo-C_{1˜6}alkyl, —C_{0˜2}alkylene-OR^{2c}, —C_{0˜2}alkylene-C(O)R^{2c}, —C_{0˜2}alkylene-C(O)NR^{2c}R^{2d}, —C_{0˜2}alkylene-NR^{2c}R^{2d}, —C_{0˜2}alkylene-NR^{2c}C(O)R^{2d}, —C_{0˜4}alkylene-S(O)₂R^{2c}, —C_{0˜2}alkylene-(3˜10 membered cycloalkyl), —C_{0˜2}alkylene-(3˜10 membered heterocycloalkyl), —C_{0˜2}alkylene-(5˜10 membered aryl ring), —C_{0˜2}alkylene-(5˜10 membered heteroaryl ring); wherein alkyl, alkylene, cycloalkyl, heterocycloalkyl, aryl ring, heteroaryl ring is optionally further substituted by one, two or three independent R^{2c};
R^{2c}, R^{2d} is each independently selected from a group consisting of hydrogen, cyano, nitro, —OH, halogen, —C_{1˜6}alkyl, halo-C_{1˜6}alkyl, —C_{0˜2}alkylene-O(C_{1˜6}alkyl), —O(halo-C_{1˜6}alkyl), —O(3˜10 membered cycloalkyl), —O(3˜10 membered heterocycloalkyl), —NH₂, —NH(C_{1˜6}alkyl), —N(C_{1˜6}alkyl) (C_{1˜6}alkyl), —C_{0˜2}alkylene-(3˜10 membered cycloalkyl), —C_{0˜2}alkylene-(3˜10 membered heterocycloalkyl), —C_{0˜2}alkylene-(5˜10 membered aryl ring), —C_{0˜2}alkylene-(5˜10 membered heteroaryl ring); wherein said alkyl, alkylene, cycloalkyl, heterocycloalkyl, aryl ring, heteroaryl ring is optionally further substituted by one, two or three independent R^{2e};
R^{2e} is each independently selected from a group consisting of hydrogen, cyano, nitro, —OH, halogen, —C_{1˜6}alkyl, halo-C_{1˜6}alkyl, —C_{0˜2}alkylene-O(C_{1˜6}alkyl), —O(halo-C_{1˜6}alkyl), —O(3˜10 membered cycloalkyl), —O(3˜10 membered heterocycloalkyl), —NH₂, —NH(C_{1˜6}alkyl), —N(C_{1˜6}alkyl) (C_{1˜6}alkyl), —C_{0˜2}alkylene-(3˜10 membered cycloalkyl), —C_{0˜2}alkylene-(3˜10 membered heterocycloalkyl), —C_{0˜2}alkylene-(5˜10 membered aryl ring), —C_{0˜2}alkylene-(5˜10 membered heteroaryl ring);
R₃ is selected from a group consisting of the following groups: hydrogen, halogen, cyano, nitro, amino, —OH, —C_{1˜6}alkyl, —O(C_{1˜6}alkyl), halo-C_{1˜6}alkyl, —C_{0˜2}alkylene-OR^{3b}, —C_{0˜2}alkylene-C(O)R^{3b}, —C_{0˜2}alkylene-C(O)NR^{3b}R^{3c}, —C_{0˜2}alkylene-NR^{3b}R^{3c}, —C_{0˜2}alkylene-NR^{3b}C(O)R^{3c}, —C_{0˜4}alkylene-S(O)₂R^{3b}, —C_{0˜2}alkylene-(3˜10 membered cycloalkyl), —C_{0˜2}alkylene-(3˜10 membered heterocycloalkyl);
R^{3b}, R^{3c} is each independently selected from a group consisting of the following groups: hydrogen, halogen, cyano, nitro, —OH, C_{1˜6}alkyl, halo-C_{1˜6}alkyl, —O(C_{1˜6}alkyl), —O(halo-C_{1˜6}alkyl), —NH₂, —NH(C_{1˜6}alkyl), —N(C_{1˜6}alkyl) (C_{1˜6}alkyl), —C_{0˜2}alkylene-(3˜10 membered cycloalkyl), —C_{0˜2}alkylene-(3˜10 membered heterocycloalkyl), —C_{0˜2}alkylene-(5˜10 membered aryl ring), —C_{0˜2}alkylene-(5˜10 membered heteroaryl ring);
R₄ is selected from a group consisting of the following groups: hydrogen, halogen, cyano, nitro, hydroxyl, —C_{1˜6}alkyl, halo-C_{1˜6}alkyl, —O(C_{1˜6}alkyl), —O(halo-C_{1˜6}alkyl), —NH₂, —NH(C_{1˜6}alkyl), —N(C_{1˜6}alkyl) (C_{1˜6}alkyl), —C_{0˜2}alkylene-(3˜10 membered cycloalkyl), —C_{0˜2}alkylene-(3˜10 membered heterocycloalkyl), —C_{0˜2}alkylene-(5˜10 membered aryl ring), —C_{0˜2}alkylene-(5˜10 membered heteroaryl ring); wherein said alkyl, alkylene, cycloalkyl, heterocycloalkyl, aryl ring, heteroaryl ring is optionally further substituted by one, two or three independent R^{4b};
R^{4b} is each independently selected from a group consisting of the following groups: hydrogen, halogen, cyano, nitro, —OH, C_{1˜6}alkyl, halo-C_{1˜6}alkyl, —O(C_{1˜6}alkyl), —O(halo-C_{1˜6}alkyl), —NH₂, —NH(C_{1˜6}alkyl), —N(C_{1˜6}alkyl) (C_{1˜6}alkyl), —C_{0˜2}alkylene-(3˜10 membered cycloalkyl), —C_{0˜2}alkylene-(3˜10 membered heterocycloalkyl), —C_{0˜2}alkylene-(5˜10 membered aryl ring), —C_{0˜2}alkylene-(5˜10 membered heteroaryl ring);
R₅ is selected from a group consisting of the following groups: hydrogen, halogen, cyano, nitro, hydroxyl, —C_{1˜6}alkyl, halo-C_{1˜6}alkyl, —O(C_{1˜6}alkyl), —O(halo-C_{1˜6}alkyl), —NH₂, —NH(C_{1˜6}alkyl), —N(C_{1˜6}alkyl) (C_{1˜6}alkyl), —C_{0˜2}alkylene-(3˜10 membered cycloalkyl), —C_{0˜2}alkylene-(3˜10 membered heterocycloalkyl), —C_{0˜2}alkylene-(5˜10 membered aryl ring), —C_{0˜2}alkylene-(5˜10 membered heteroaryl ring);
R₆ is selected from a group consisting of the following groups: hydrogen, halogen, cyano, nitro, hydroxyl, —C_{1˜6}alkyl, halo-C_{1˜6}alkyl, —O(C_{1˜6}alkyl), —O(halo-C_{1˜6}alkyl), —NH₂, —NH(C_{1˜6}alkyl), —N(C_{1˜6}alkyl) (C_{1˜6}alkyl), —C_{0˜2}alkylene-(3˜10 membered cycloalkyl), —C_{0˜2}alkylene-(3˜10 membered heterocycloalkyl), —C_{0˜2}alkylene-(5˜10 membered aryl ring), —C_{0˜2}alkylene-(5˜10 membered heteroaryl ring);
R₇ is selected from a group consisting of the following groups: hydrogen, —C_{1˜6}alkyl, halo-C_{1˜6}alkyl, —C_{0˜2}alkylene-(3˜10 membered cycloalkyl), —C_{0˜2}alkylene-(3˜10 membered heterocycloalkyl), —C_{0˜2}alkylene-(5˜10 membered aryl ring), —C_{0˜2}alkylene-(5˜10 membered heteroaryl ring);
R₈ is selected from a group consisting of the following groups: hydrogen, halogen, cyano, nitro, —OH, C_{1˜6}alkyl, halo-C_{1˜6}alkyl, —O(C_{1˜6}alkyl), —O(halo-C_{1˜6}alkyl), —NH₂, —NH(C_{1˜6}alkyl), —N(C_{1˜6}alkyl) (C_{1˜6}alkyl), —C_{0˜2}alkylene-(3˜10 membered cycloalkyl), —C_{0˜2}alkylene-(3˜10 membered heterocycloalkyl), —C_{0˜2}alkylene-(5˜10 membered aryl ring), —C_{0˜2}alkylene-(5˜10 membered heteroaryl ring);
or, R⁷, R⁸ and the atom directly bound thereto form a 4-10 membered heterocyclic ring; wherein said heterocyclic ring is optionally further substituted by one, two or three independent R⁷¹;
each R⁷¹ is each independently selected from a group consisting of the following groups: hydrogen, halogen, cyano, nitro, —OH, C_{1˜6}alkyl, halo-C_{1˜6}alkyl, —O(C_{1˜6}alkyl), —O(halo-C_{1˜6}alkyl), —NH₂, —NH(C_{1˜6}alkyl), —N(C_{1˜6}alkyl) (C_{1˜6}alkyl), —C_{0˜2}alkylene-(3˜10 membered cycloalkyl), —C_{0˜2}alkylene-(3˜10 membered heterocycloalkyl), —C_{0˜2}alkylene-(5˜10 membered aryl ring), —C_{0˜2}alkylene-(5˜10 membered heteroaryl ring).

2. The compound, or a deuterated compound thereof, or a stereoisomer thereof, or a pharmaceutically acceptable salt thereof according to claim 1, wherein said R₁, R₃, R₄, R₅, R₆, R₇, Rₐ is each independently selected from a group consisting of hydrogen, halogen, —C_{1˜3}alkyl, halo-C_{1˜3}alkyl.

3. The compound, or a deuterated compound thereof, or a stereoisomer thereof, or a pharmaceutically acceptable salt thereof according to claim 2, wherein said R₁, R₃, R₄, R₅, R₆, R₇, Rₐ is each independently selected from a group consisting of hydrogen, Cl, methyl, ethyl, —CF₃.

4. The compound, or a deuterated compound thereof, or a stereoisomer thereof, or a pharmaceutically acceptable salt thereof according to claim 1, wherein:
R₂ is selected from a group consisting of -(3˜10 membered cycloalkyl), -(3˜10 membered heterocycloalkyl); wherein said cycloalkyl, heterocycloalkyl is optionally further substituted by one, two or three independent R^{2b};
R^{2b} is selected from a group consisting of hydrogen, halogen, —C_{1˜6}alkyl, —NR^{2c}R^{2d}, —NR^{2c}C(O)R^{2d}, —C_{0˜2}alkylene-(3˜10 membered cycloalkyl), —C_{0˜2}alkylene-(3˜10 membered heterocycloalkyl); wherein said alkyl, cycloalkyl, heterocycloalkyl is optionally further substituted by one, two or three independent R^{2c};
R^{2c}, R^{2d} is each independently selected from a group consisting of hydrogen, halogen, —C_{1˜6}alkyl, halo-C_{1˜6}alkyl, —O(C_{1˜6}alkyl), —O(halo-C_{1˜6}alkyl), —O(3˜10 membered cycloalkyl), -(3˜10 membered cycloalkyl), -(3˜10 membered heterocycloalkyl); wherein said alkyl, cycloalkyl, heterocycloalkyl is optionally further substituted by one, two or three independent R^{2e};
R^{2e} is each independently selected from a group consisting of hydrogen, halogen, —O(C_{1˜6}alkyl);
R₂ is selected from a group consisting of

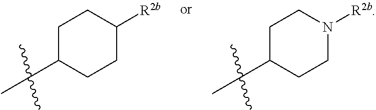

5. The compound, or a deuterated compound thereof, or a stereoisomer thereof, or a pharmaceutically acceptable salt thereof according to claim 4, wherein:
R₂ is specifically selected from a group consisting of

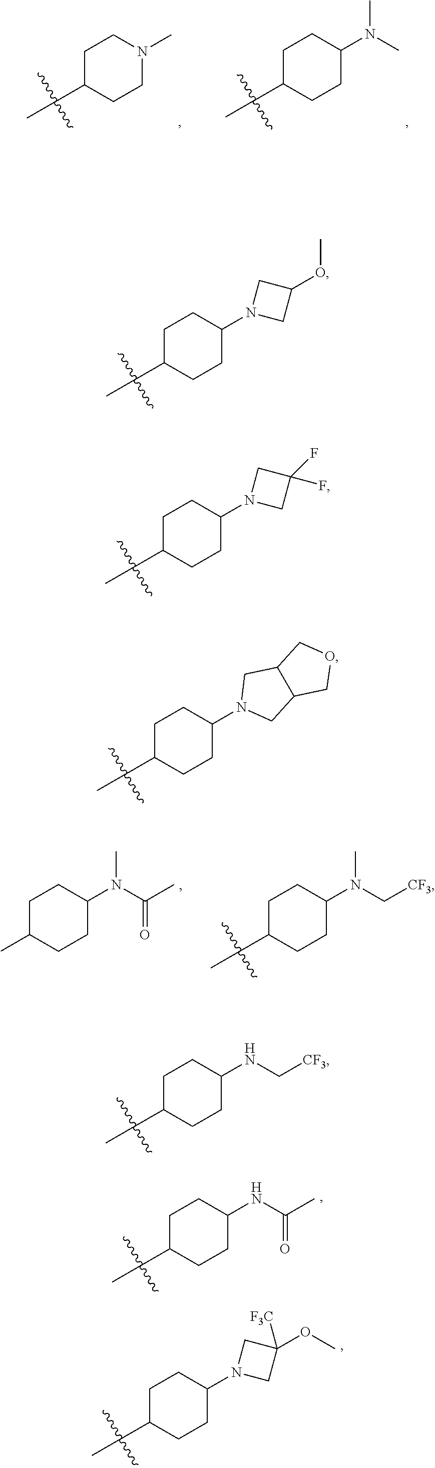

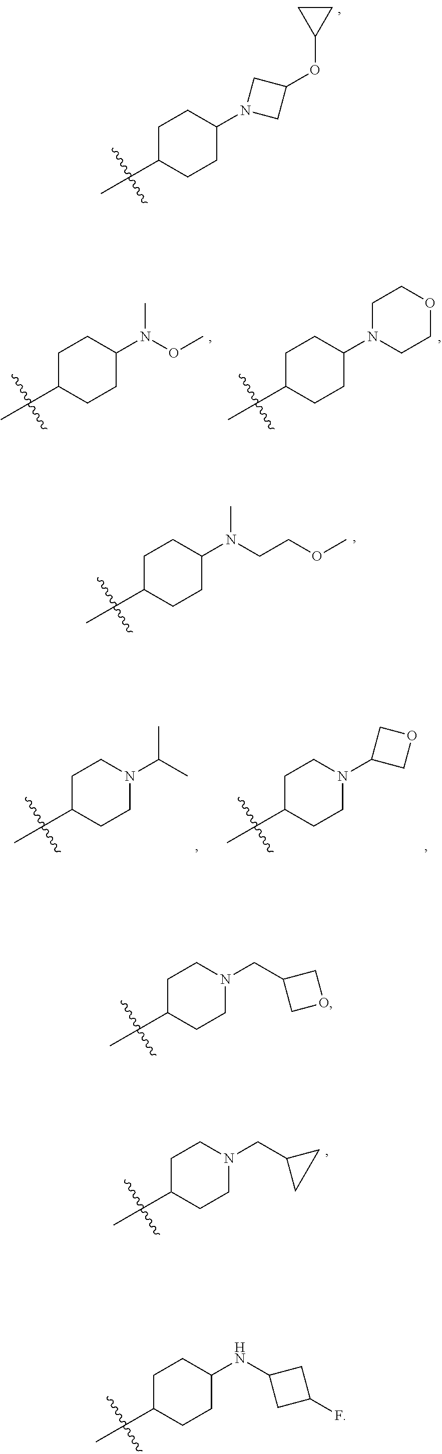

6. The compound, or a deuterated compound thereof, or a stereoisomer thereof, or a pharmaceutically acceptable salt thereof according to claim 1, wherein:

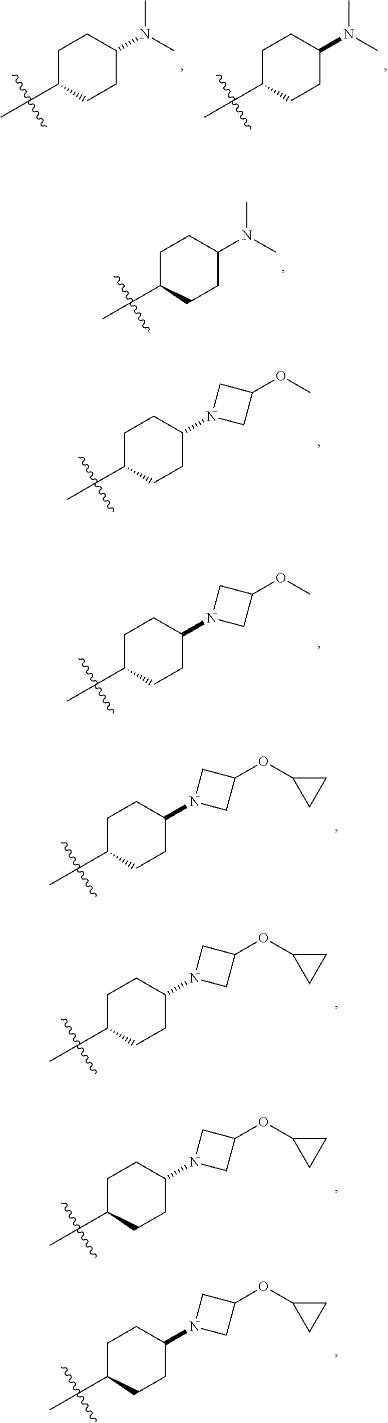

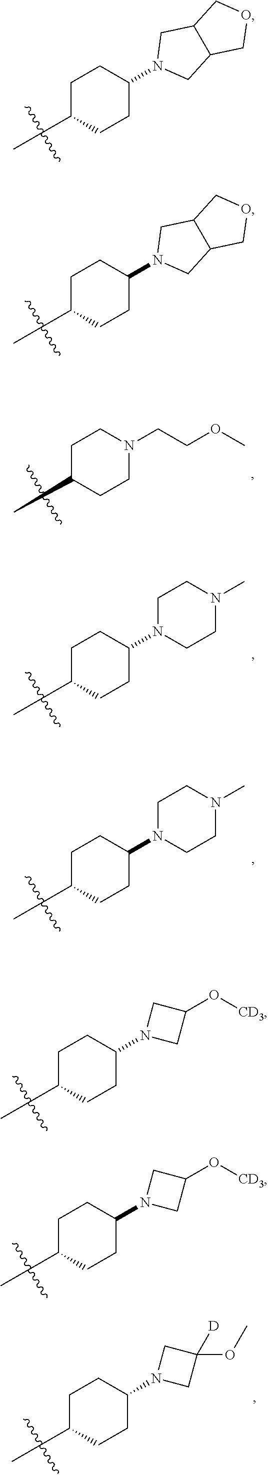

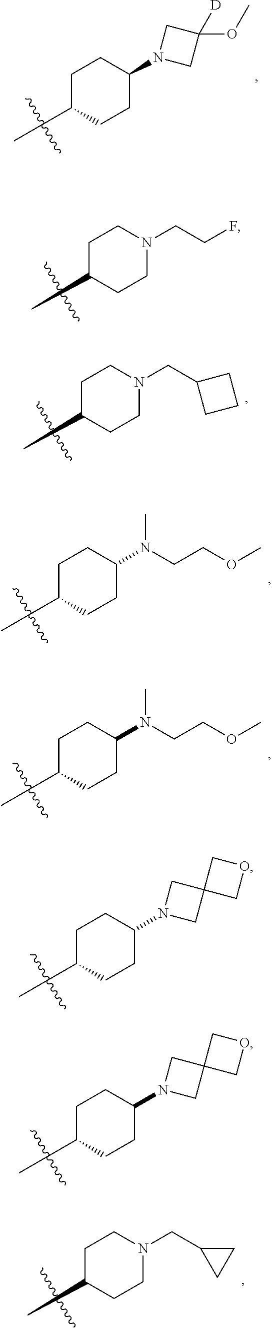

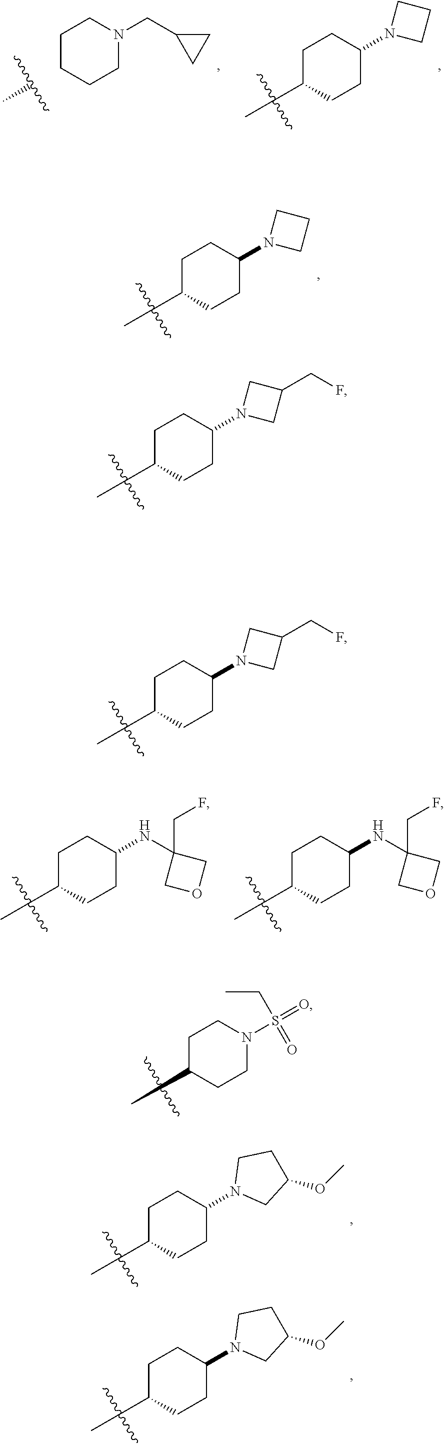

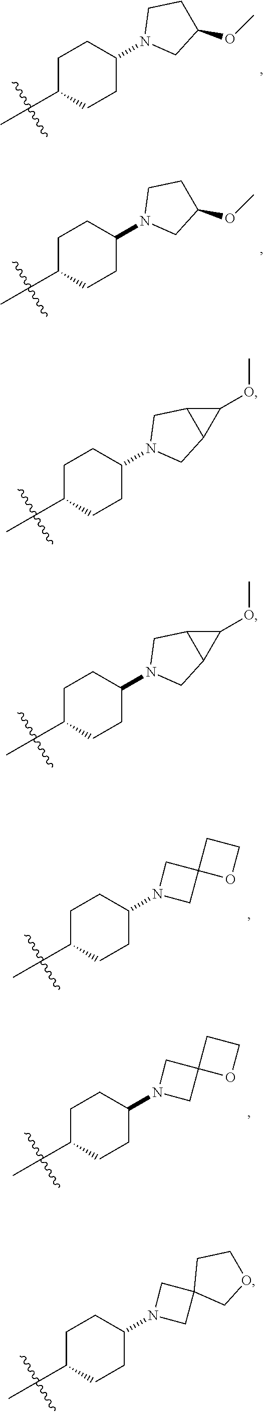

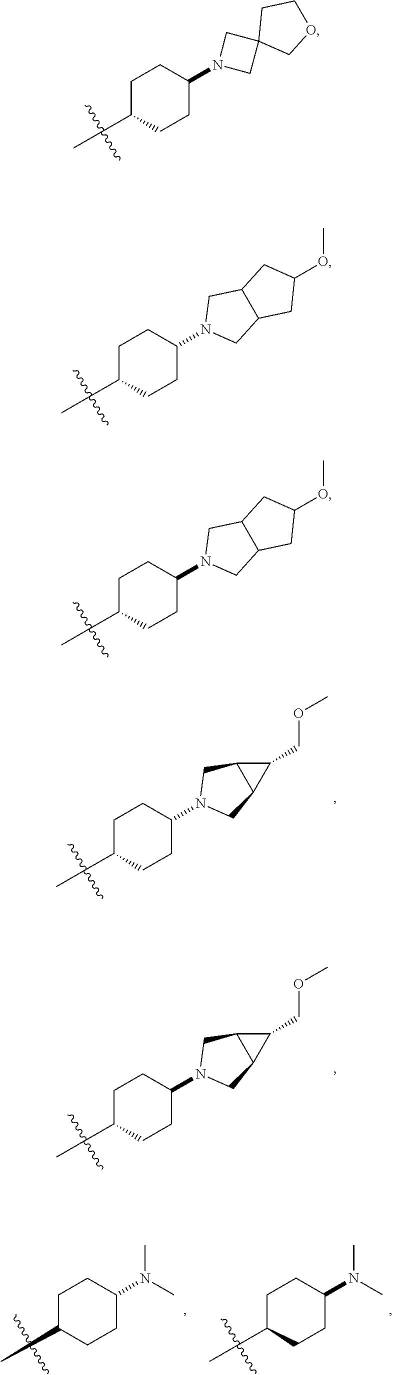

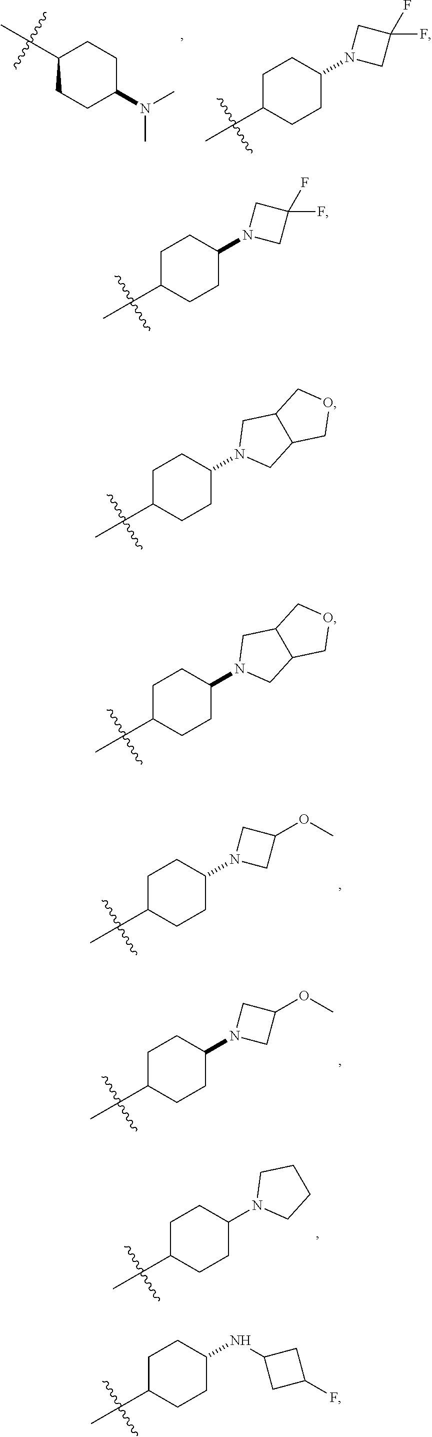

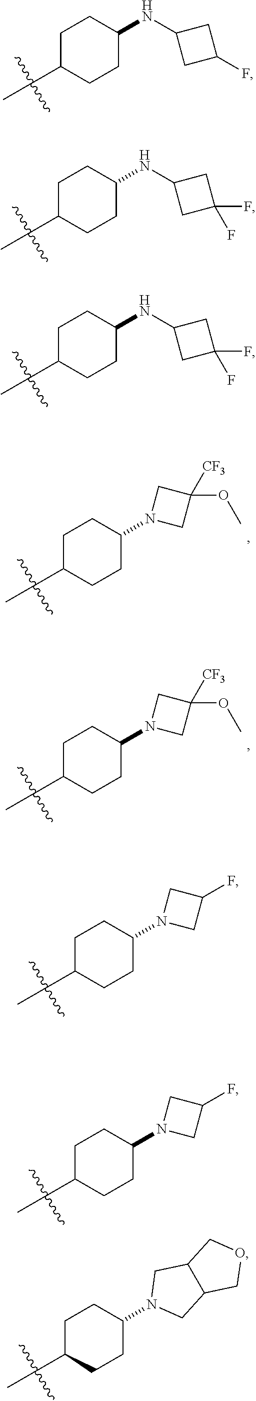

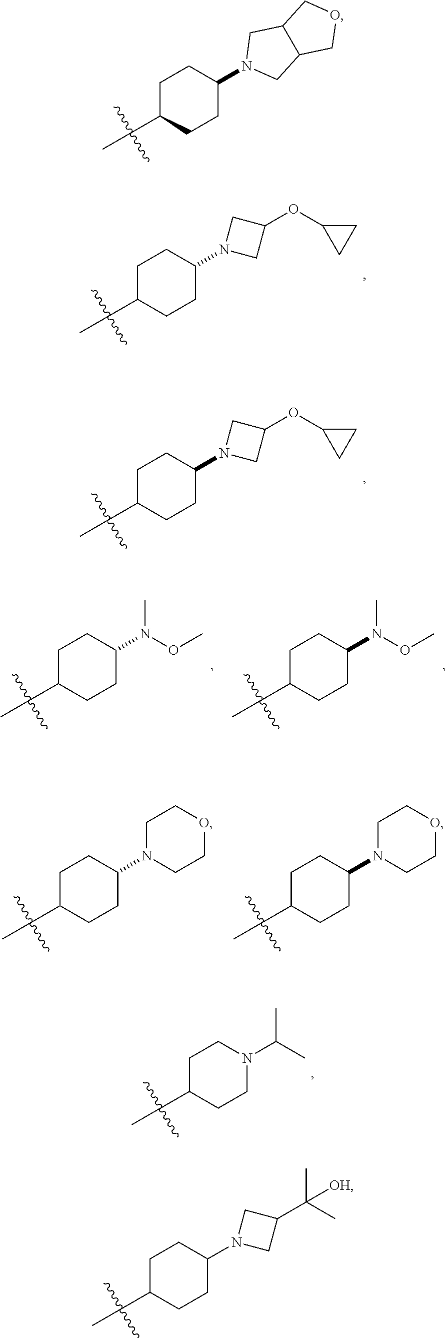

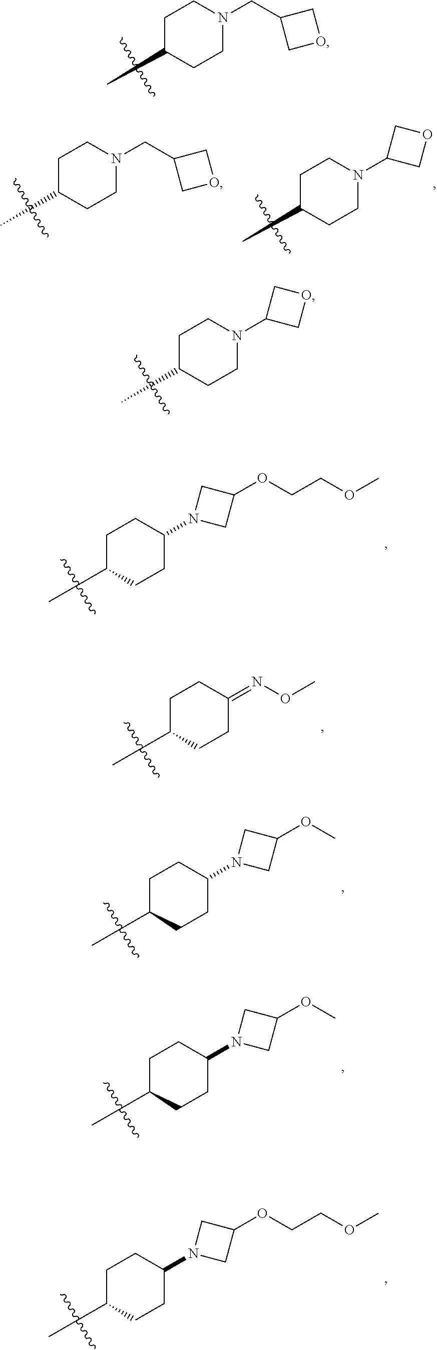

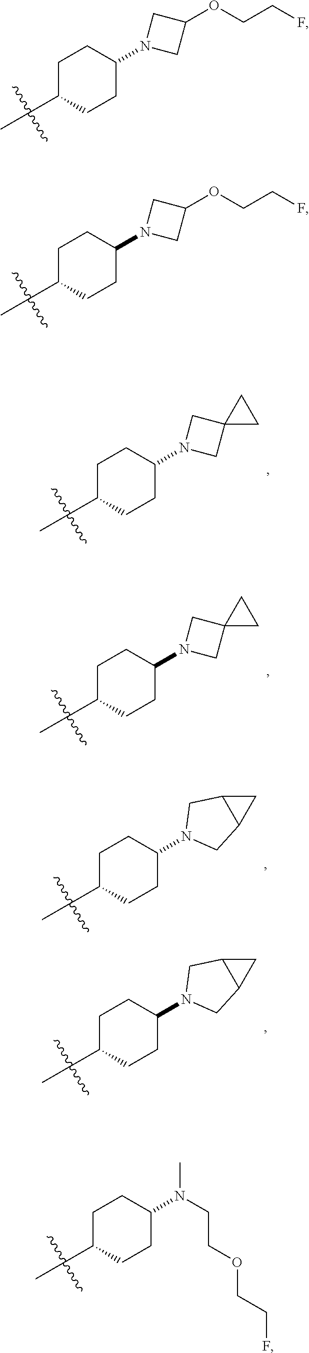

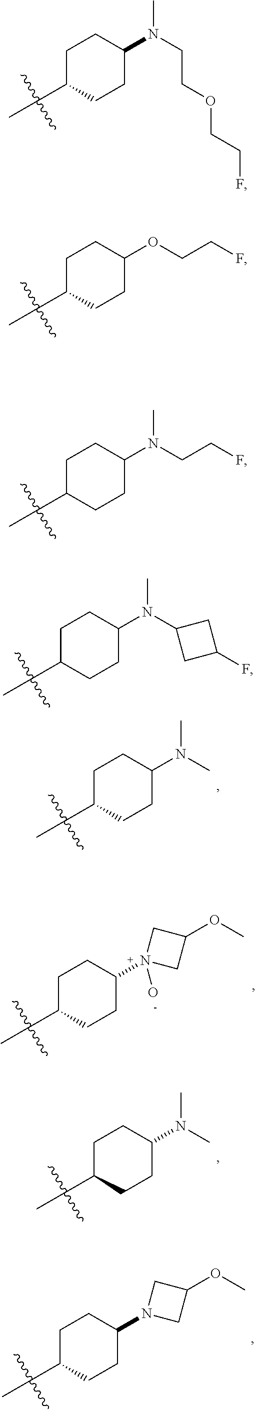

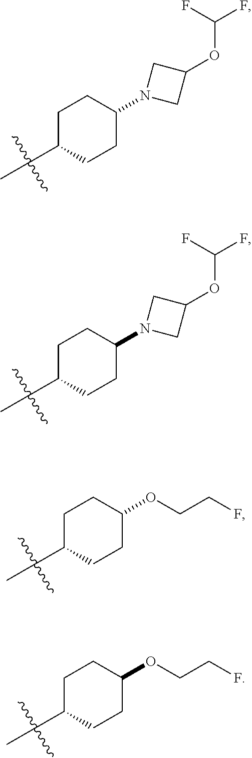 [SMILES: *[C@H]1CC[C@@H](N2CC(OC(F)F)C2)CC1 ; *[C@H]1CC[C@H](N2CC(OC(F)F)C2)CC1 ; *[C@H]1CC[C@@H](OCCF)CC1 ; *[C@H]1CC[C@H](OCCF)CC1]

7. The compound, or a deuterated compound thereof, or a stereoisomer thereof, or a pharmaceutically acceptable salt thereof according to claim 1, wherein:
X is selected from a group consisting of single bond, O, CHR^{X2}; Y is selected from a group consisting of O, NR^{Y1} or CHR^{Y2}; Z is selected from a group consisting of —C═O, O, NR^{Z1} or CHR^{Z2}; Q is selected from a group consisting of single bond, O or S;
R^{X2} is selected from a group consisting of hydrogen, —C_{1˜3}alkyl;
R^{Y1} is selected from a group consisting of hydrogen, —C_{1˜3}alkyl, halo-C₁₋₃alkyl;
R^{Y2} is selected from a group consisting of hydrogen, —C_{1˜3}alkyl, halo-C₁₋₃alkyl;
R^{Z1} is selected from a group consisting of hydrogen, —C_{1˜3}alkyl, -(3˜6 membered cycloalkyl), halo-C_{1˜3}alkyl;
R^{Z2} is selected from a group consisting of hydrogen, —C_{1˜3}alkyl, halo-C_{1˜3}alkyl.

8. The compound, or a deuterated compound thereof, or a stereoisomer thereof, or a pharmaceutically acceptable salt thereof according to claim 7, wherein:
R^{Y1} is selected from a group consisting of methyl; R^{Y2} is selected from a group consisting of hydrogen, methyl; R^{Z1} is selected from a group consisting of hydrogen, methyl, ethyl, cyclopropyl.

9. The compound, or a deuterated compound thereof, or a stereoisomer thereof, or a pharmaceutically acceptable salt thereof according to claim 1, including the following compounds represented by Formula Ila: 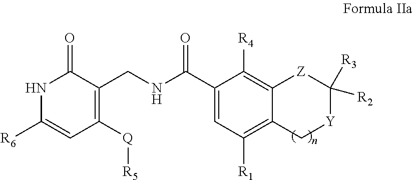
Q is selected from a group consisting of single bond, O or S;
Z is selected from a group consisting of C═O, O, NR^{Z1} or CHR^{Z2};
Y is selected from a group consisting of O, NR^{Y1} or CHR^{Y2}; n is 0 or 1;
when n is 0, Y and Z are not both O;
R^{Y1} is selected from a group consisting of hydrogen, —C_{1˜6}alkyl, halo-C_{1˜6}alkyl, —C_{0˜2}alkylene-(3˜10 membered cycloalkyl), —C_{0˜2}alkylene-(3˜10 membered heterocycloalkyl), —C_{0˜2}alkylene-(5˜10 membered aryl ring), —C_{0˜2}alkylene-(5˜10 membered heteroaryl ring);
R^{Y2} is selected from a group consisting of hydrogen, halogen, cyano, hydroxyl, —C_{1˜6}alkyl, halo-C_{1˜6}alkyl, —O(C_{1˜6}alkyl), —O(halo-C_{1˜6}alkyl), —NH₂, —NH(C_{1˜6}alkyl), —N(C_{1˜6}alkyl) (C_{1˜6}alkyl), —C_{0˜2}alkylene-(3˜10 membered cycloalkyl), —C_{0˜2}alkylene-(3˜10 membered heterocycloalkyl), —C_{0˜2}alkylene-(5˜10 membered aryl ring), —C_{0˜2}alkylene-(5˜10 membered heteroaryl ring);
R^{Z1} is selected from a group consisting of hydrogen, —C_{1˜6}alkyl, halo-C_{1˜6}alkyl, —C_{0˜2}alkylene-(3˜10 membered cycloalkyl), —C_{0˜2}alkylene-(3˜10 membered heterocycloalkyl), —C_{0˜2}alkylene-(5˜10 membered aryl ring), —C_{0˜2}alkylene-(5˜10 membered heteroaryl ring);
R^{Z2} is selected from a group consisting of hydrogen, halogen, cyano, hydroxyl, —C_{1˜6}alkyl, halo-C_{1˜6}alkyl, —O(C_{1˜6}alkyl), —O(halo-C_{1˜6}alkyl), —NH₂, —NH(C_{1˜6}alkyl), —N(C_{1˜6}alkyl) (C_{1˜6}alkyl), —C_{0˜2}alkylene-(3˜10 membered cycloalkyl), —C_{0˜2}alkylene-(3˜10 membered heterocycloalkyl), —C_{0˜2}alkylene-(5˜10 membered aryl ring), —C_{0˜2}alkylene-(5˜10 membered heteroaryl ring);
R₁ is selected from a group consisting of hydrogen, halogen, cyano, nitro, amino, hydroxyl, —C_{1˜6}alkyl, halo-C_{1˜6}alkyl, —C_{0˜2}alkylene-OR^{1b}, —C_{0˜2}alkylene-C(O)R^{1b}, —C_{0˜2}alkylene-C(O)NR^{1b}R^{1c}, —C_{0˜2}alkylene-NR^{1b}R^{1c}, —C_{0˜2}alkylene-NR^{1b}C(O)R^{1c}, —C_{0˜4}alkylene-S(O)₂R^{1b}, —C_{0˜2}alkylene-(3˜10 membered cycloalkyl), —C_{0˜2}alkylene-(3˜10 membered heterocycloalkyl), —C_{0˜2}alkylene-(5˜10 membered aryl ring), —C_{0˜2}alkylene-(5˜10 membered heteroaryl ring); wherein said alkyl, alkylene, cycloalkyl, heterocycloalkyl, aryl ring, heteroaryl ring is optionally further substituted by one, two or three independent R^{1b};
R^{1b}, R^{1c} is each independently selected from a group consisting of hydrogen, —OH, C_{1˜6}alkyl, halo-C_{1˜6}alkyl, —O(C_{1˜6}alkyl), —O(halo-C_{1˜6}alkyl), —NH₂, —NH(C_{1˜6}alkyl), —N(C_{1˜6}alkyl) (C_{1˜6}alkyl), —C_{0˜2}alkylene-(3˜10 membered cycloalkyl), —C_{0˜2}alkylene-(3˜10 membered heterocycloalkyl), —C_{0˜2}alkylene-(5˜10 membered aryl ring), —C_{0˜2}alkylene-(5˜10 membered heteroaryl ring);
R₂ is selected from a group consisting of —C_{0˜2}alkylene-(3˜10 membered cycloalkyl), —C_{0˜2}alkylene-(3˜10 membered heterocycloalkyl); wherein said alkylene, cycloalkyl, heterocycloalkyl is optionally further substituted by one, two or three independent R^{2b};
R^{2b} is each independently selected from a group consisting of hydrogen, halogen, cyano, nitro, amino, hydroxyl, —C_{1˜6}alkyl, —O(C_{1˜6}alkyl), halo-C_{1˜6}alkyl, —C_{0˜2}alkylene-OR², —C_{0˜2}alkylene-C(O)R^{2c}, —C_{0˜2}alkylene-C(O)NR^{2c}R^{2d}, —C_{0˜2}alkylene-NR^{2c}R^{2d}, —C_{0˜2}alkylene-NR^{2c}C(O)R^{2d}, —C_{0˜4}alkylene-S(O)₂R^{2c}, —C_{0˜2}alkylene-(3˜10 membered cycloalkyl), —C_{0˜2}alkylene-(3˜10 membered heterocycloalkyl), —C_{0˜2}alkylene-(5˜10 membered aryl ring), —C_{0˜2}alkylene-(5˜10 membered heteroaryl ring); wherein alkyl, alkylene, cycloalkyl, heterocycloalkyl, aryl ring, heteroaryl ring is optionally further substituted by one, two or three independent R^{2c};
R^{2c}, R^{2d} is each independently selected from a group consisting of hydrogen, cyano, nitro, —OH, halogen, —C_{1˜6}alkyl, halo-C_{1˜6}alkyl, —C_{0˜2}alkylene-O(C_{1˜6}alkyl), —O(halo-C_{1˜6}alkyl), —O(3˜10 membered cycloalkyl), —O(3˜10 membered heterocycloalkyl), —NH₂, —NH(C_{1˜6}alkyl), —N(C_{1˜6}alkyl) (C_{1˜6}alkyl), —C_{0˜2}alkylene-(3˜10 membered cycloalkyl), —C_{0˜2}alkylene-(3˜10 membered heterocycloalkyl), —C_{0˜2}alkylene-(5˜10 membered aryl ring), —C_{0˜2}alkylene-(5˜10 membered heteroaryl ring); wherein said alkyl, alkylene, cycloalkyl, heterocycloalkyl, aryl ring, heteroaryl ring is optionally further substituted by one, two or three independent R^{2e};
R^{2e} is each independently selected from a group consisting of hydrogen, cyano, nitro, —OH, halogen, —C_{1˜6}alkyl, halo-C_{1˜6}alkyl, —C_{0˜2}alkylene-O(C_{1˜6}alkyl), —O(halo-C_{1˜6}alkyl), —O(3˜10 membered cycloalkyl), —O(3˜10 membered heterocycloalkyl), —NH₂, —NH(C_{1˜6}alkyl), —N(C_{1˜6}alkyl) (C_{1˜6}alkyl), —C_{0˜2}alkylene-(3˜10 membered cycloalkyl), —C_{0˜2}alkylene-(3˜10 membered heterocycloalkyl), —C_{0˜2}alkylene-(5˜10 membered aryl ring), —C_{0˜2}alkylene-(5˜10 membered heteroaryl ring);
R₃ is selected from a group consisting of hydrogen, halogen, cyano, nitro, amino, —OH, —C_{1˜6}alkyl, —O(C_{1˜6}alkyl), halo-C_{1˜6}alkyl, —C_{0˜2}alkylene-OR^{3b}, —C_{0˜2}alkylene-C(O)R^{3b}, —C_{0˜2}alkylene-C(O)NR^{3b}R^{3c}, —C_{0˜2}alkylene-NR^{3b}R^{3c}, —C_{0˜2}alkylene-NR^{3b}C(O)R^{3c}, —C_{0˜4}alkylene-S(O)₂R^{3b}, —C_{0˜2}alkylene-(3˜10 membered cycloalkyl), —C_{0˜2}alkylene-(3˜10 membered heterocycloalkyl);
R^{3b}, R^{3c} is each independently selected from a group consisting of hydrogen, halogen, cyano, nitro, —OH, C_{1˜6}alkyl, halo-C_{1˜6}alkyl, —O(C_{1˜6}alkyl), —O(halo-C_{1˜6}alkyl), —NH₂, —NH(C_{1˜6}alkyl), —N(C_{1˜6}alkyl) (C_{1˜6}alkyl), —C_{0˜2}alkylene-(3˜10 membered cycloalkyl), —C_{0˜2}alkylene-(3˜10 membered heterocycloalkyl), —C_{0˜2}alkylene-(5˜10 membered aryl ring), —C_{0˜2}alkylene-(5˜10 membered heteroaryl ring);
R₄ is selected from a group consisting of hydrogen, halogen, cyano, nitro, hydroxyl, —C_{1˜6}alkyl, halo-C_{1˜6}alkyl, —O(C_{1˜6}alkyl), —O(halo-C_{1˜6}alkyl), —NH₂, —NH(C_{1˜6}alkyl), —N(C_{1˜6}alkyl) (C_{1˜6}alkyl), —C_{0˜2}alkylene-(3˜10 membered cycloalkyl), —C_{0˜2}alkylene-(3˜10 membered heterocycloalkyl), —C_{0˜2}alkylene-(5˜10 membered aryl ring), —C_{0˜2}alkylene-(5˜10 membered heteroaryl ring); wherein said alkyl, alkylene, cycloalkyl, heterocycloalkyl, aryl ring, heteroaryl ring is optionally further substituted by one, two or three independent R^{4b};
R^{4b} is each independently selected from a group consisting of hydrogen, halogen, cyano, nitro, —OH, C_{1˜6}alkyl, halo-C_{1˜6}alkyl, —O(C_{1˜6}alkyl), —O(halo-C_{1˜6}alkyl), —NH₂, —NH(C_{1˜6}alkyl), —N(C_{1˜6}alkyl) (C_{1˜6}alkyl), —C_{0˜2}alkylene-(3˜10 membered cycloalkyl), —C_{0˜2}alkylene-(3˜10 membered heterocycloalkyl), —C_{0˜2}alkylene-(5˜10 membered aryl ring), —C_{0˜2}alkylene-(5˜10 membered heteroaryl ring);
R₅ is selected from a group consisting of hydrogen, halogen, cyano, nitro, hydroxyl, —C_{1˜6}alkyl, halo-C_{1˜6}alkyl, —O(C_{1˜6}alkyl), —O(halo-C_{1˜6}alkyl), —NH₂, —NH(C_{1˜6}alkyl), —N(C_{1˜6}alkyl) (C_{1˜6}alkyl), —C_{0˜2}alkylene-(3˜10 membered cycloalkyl), —C_{0˜2}alkylene-(3˜10 membered heterocycloalkyl), —C_{0˜2}alkylene-(5˜10 membered aryl ring), —C_{0˜2}alkylene-(5˜10 membered heteroaryl ring);
R₆ is selected from a group consisting of hydrogen, halogen, cyano, nitro, hydroxyl, —C_{1˜6}alkyl, halo-C_{1˜6}alkyl, —O(C_{1˜6}alkyl), —O(halo-C_{1˜6}alkyl), —NH₂, —NH(C_{1˜6}alkyl), —N(C_{1˜6}alkyl) (C_{1˜6}alkyl), —C_{0˜2}alkylene-(3˜10 membered cycloalkyl), —C_{0˜2}alkylene-(3˜10 membered heterocycloalkyl), —C_{0˜2}alkylene-(5˜10 membered aryl ring), —C_{0˜2}alkylene-(5˜10 membered heteroaryl ring).

10. The compound, or a deuterated compound thereof, or a stereoisomer thereof, or a pharmaceutically acceptable salt thereof according to claim 9, wherein said R₁, R₃, R₄, R₅, R₆ is each independently selected from a group consisting of hydrogen, halogen, —C_{1˜3}alkyl;
Q is selected from a group consisting of single bond, O or S; Z is selected from a group consisting of C═O, O, NR^{Z1} or CHR^{Z2}; Y is selected from a group consisting of O or NR^{Y1};
R^{Z1} is selected from a group consisting of hydrogen, —C_{1˜3}alkyl, -(3˜6 membered cycloalkyl), halo-C_{1˜3}alkyl;
R^{Z2} is selected from a group consisting of hydrogen, —C_{1˜3}alkyl, halo-C_{1˜3}alkyl;
R^{Y1} is selected from a group consisting of hydrogen, —C_{1˜3}alkyl, halo-C_{1˜3}alkyl.

11. The compound, or a deuterated compound thereof, or a stereoisomer thereof, or a pharmaceutically acceptable salt thereof according to claim 9, wherein:
said R₂ is specifically selected from a group consisting of 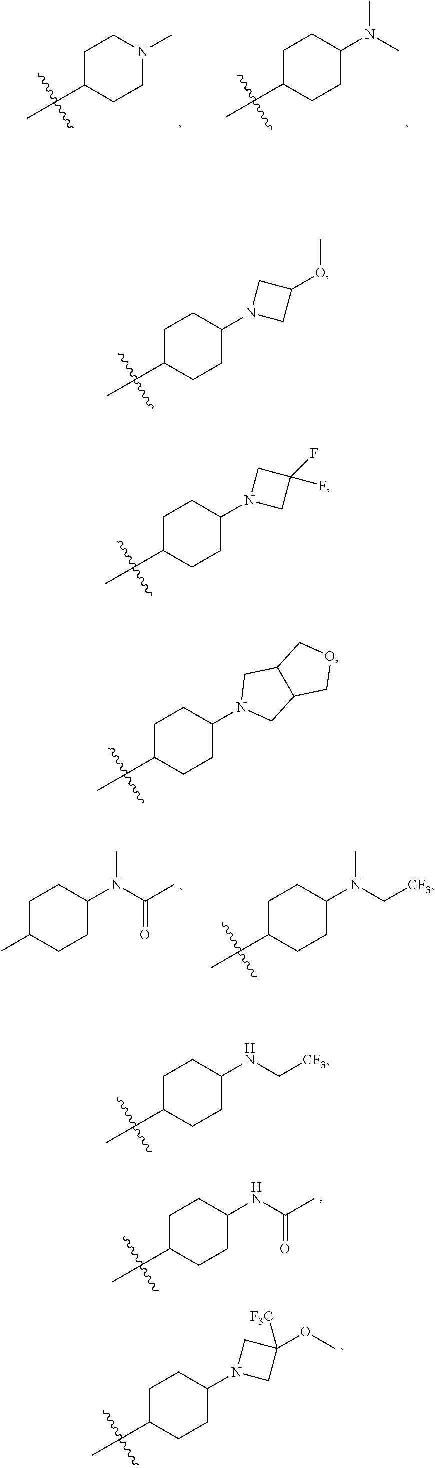 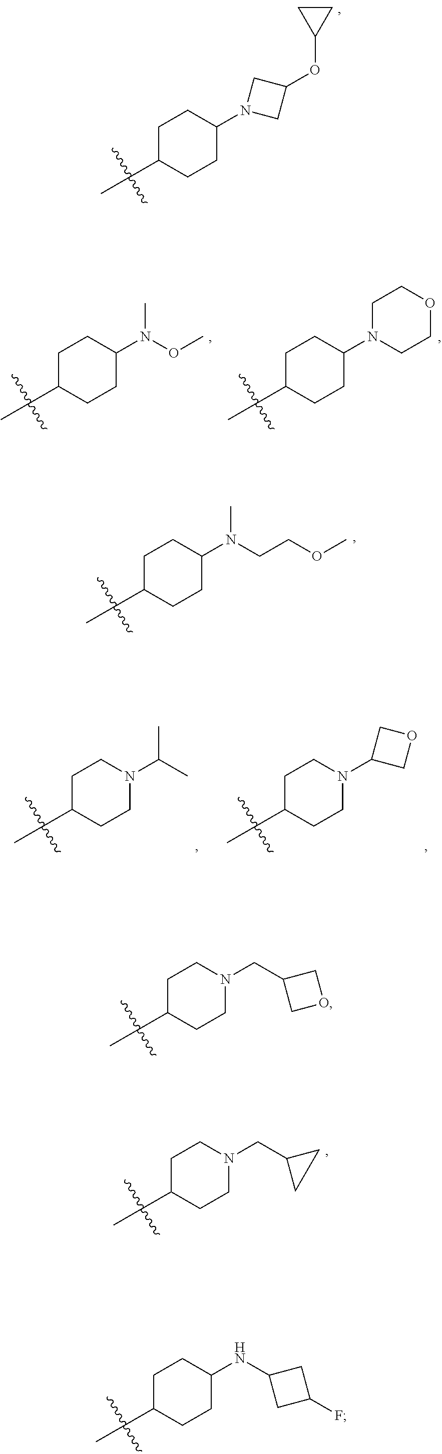
preferably, said R₂ is specifically selected from a group consisting of

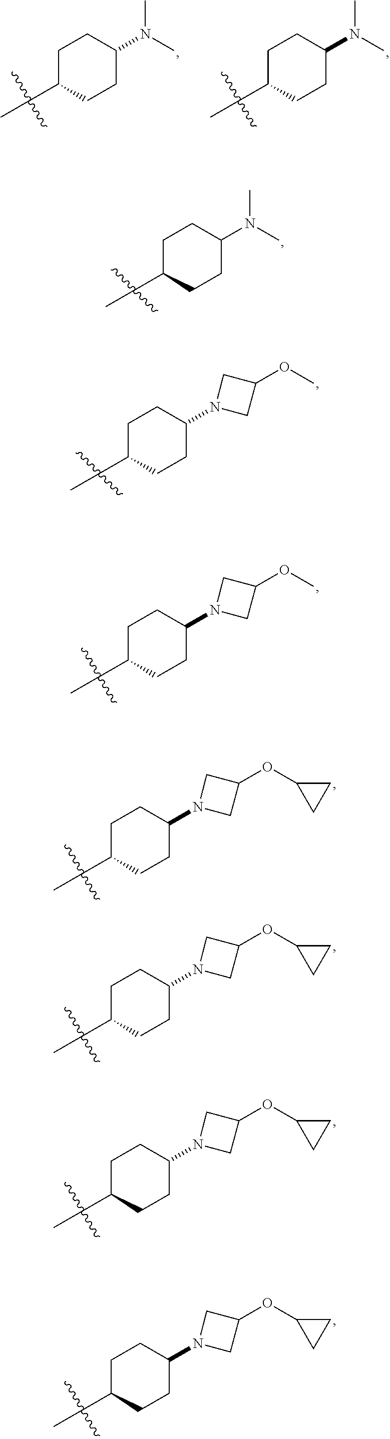

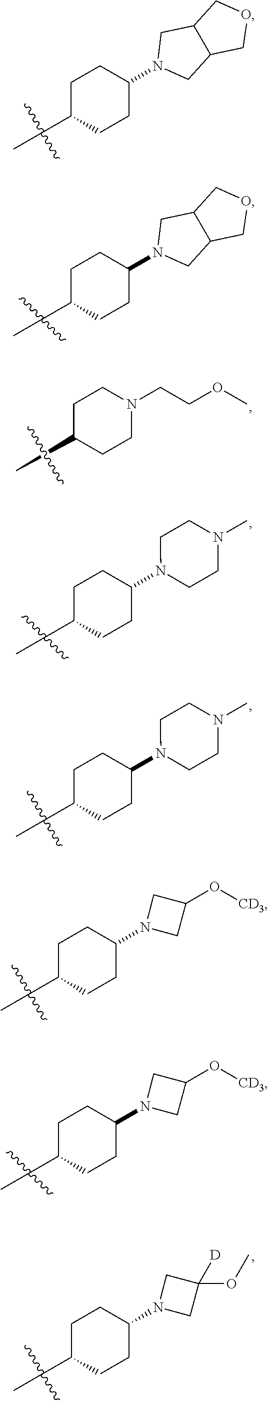

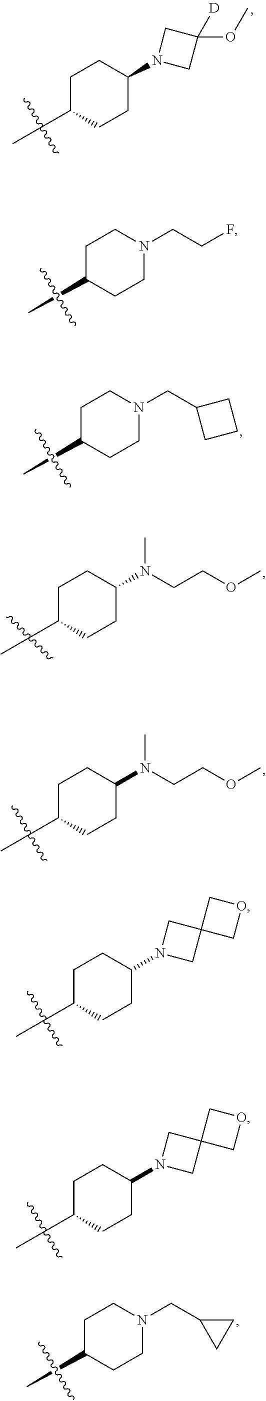

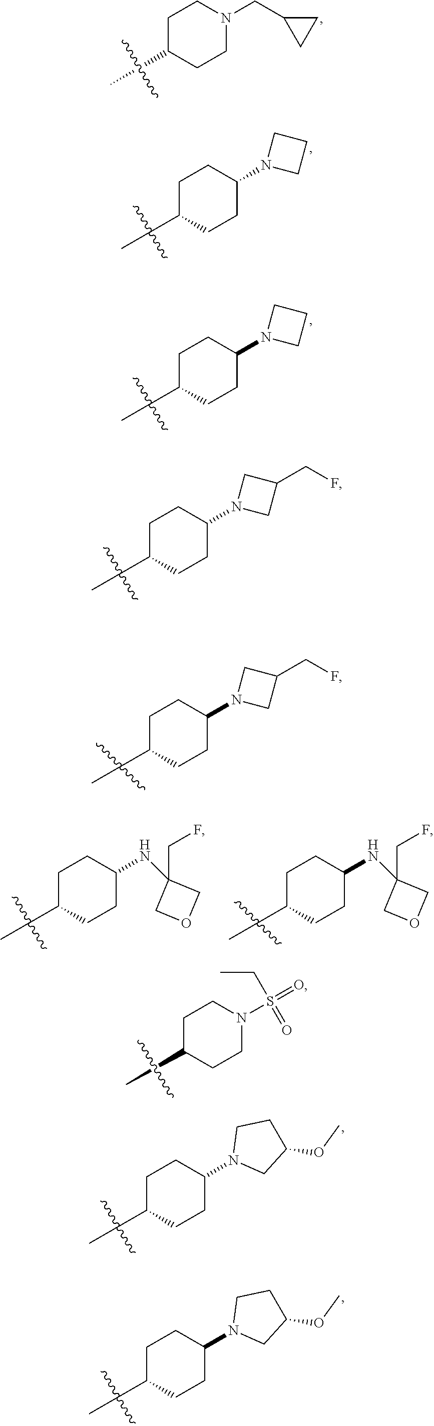

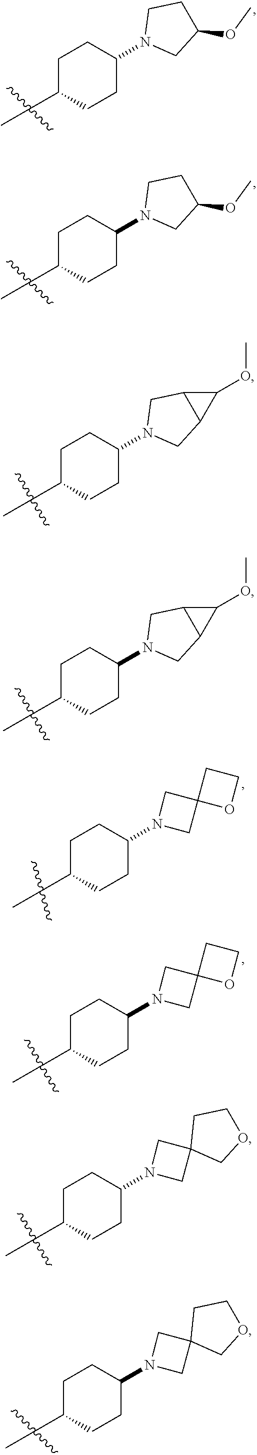

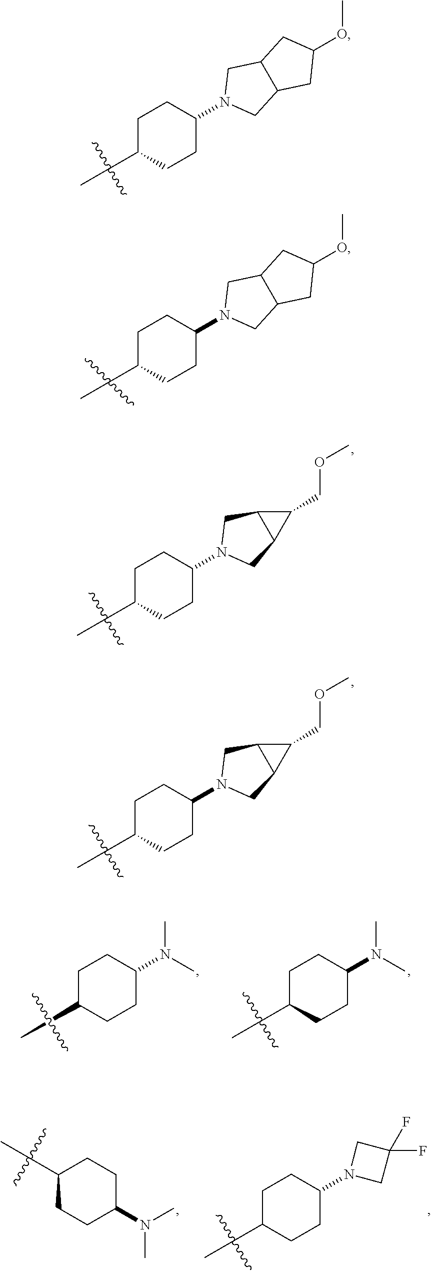

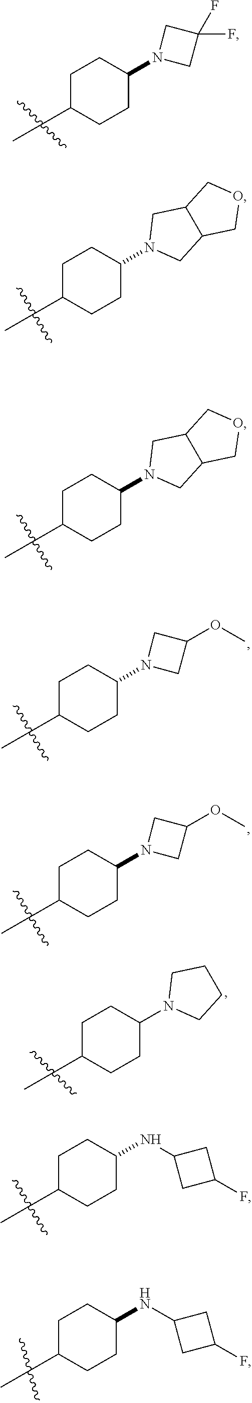

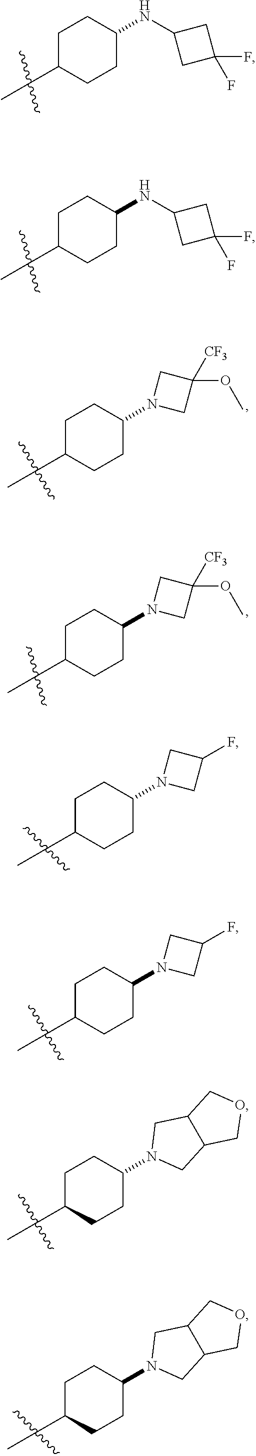

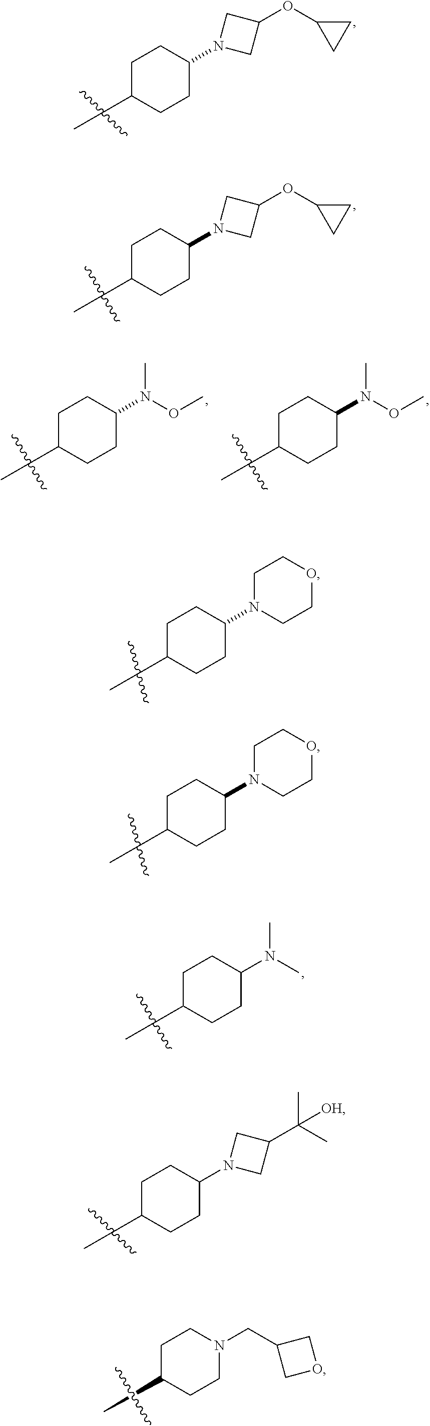

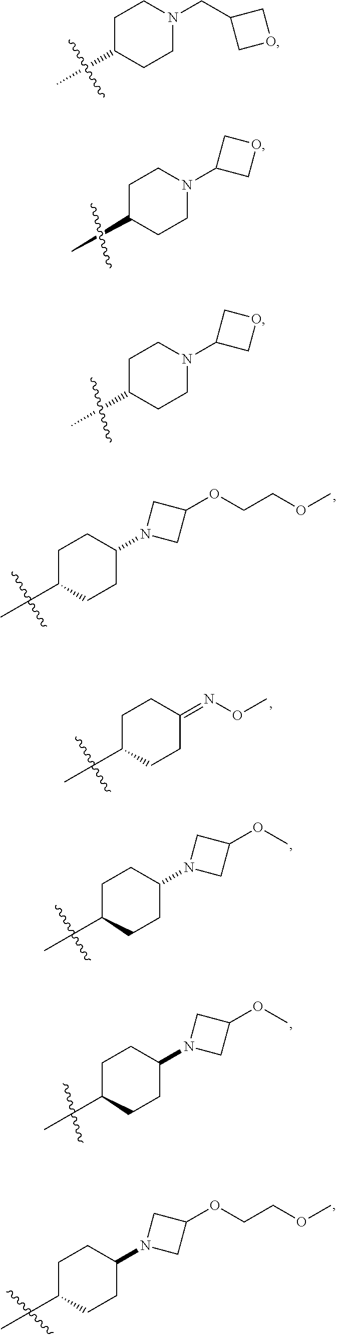

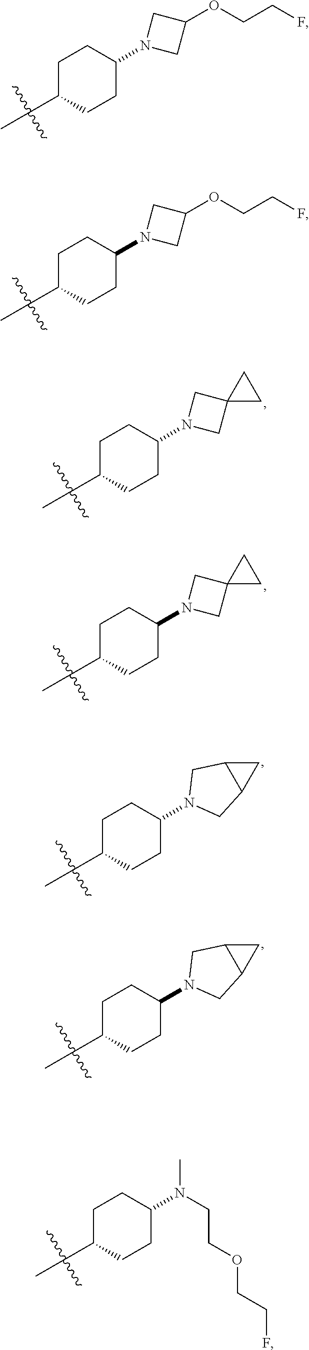

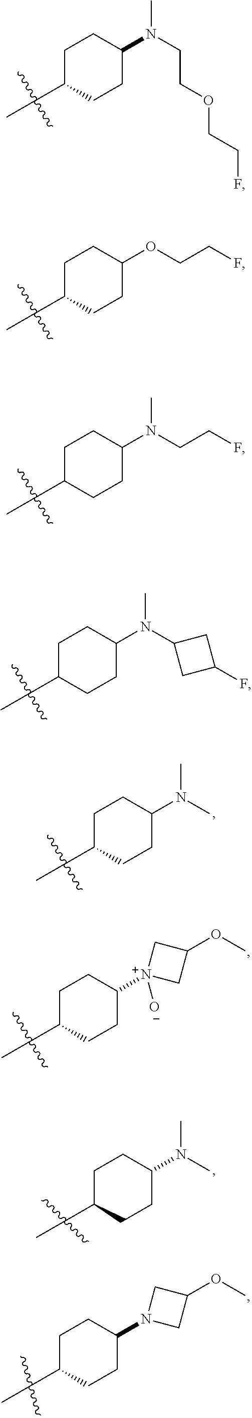

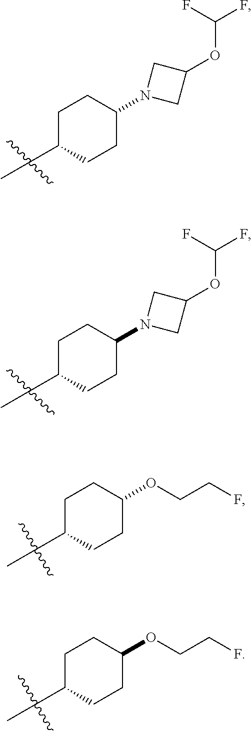 [SMILES: *[C@H]1CC[C@@H](N2CC(OC(F)F)C2)CC1 ; *[C@H]1CC[C@H](N2CC(OC(F)F)C2)CC1 ; *[C@H]1CC[C@@H](OCCF)CC1 ; *[C@H]1CC[C@H](OCCF)CC1]

12. The compound, or a deuterated compound thereof, or a stereoisomer thereof, or a pharmaceutically acceptable salt thereof according to claim 1, including the following compounds represented by Formula IIb: 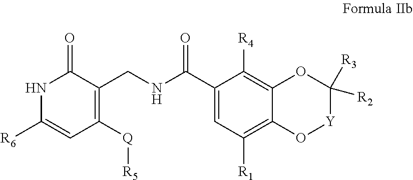
Q is selected from a group consisting of single bond, O or S; Y is selected from a group consisting of O, NR^{Y1} or CHR^{Y2};
R^{Y1} is selected from a group consisting of hydrogen, —C_{1˜6}alkyl, halo-C_{1˜6}alkyl, —C_{0˜2}alkylene-(3˜10 membered cycloalkyl), —C_{0˜2}alkylene-(3˜10 membered heterocycloalkyl), —C_{0˜2}alkylene-(5˜10 membered aryl ring), —C_{0˜2}alkylene-(5˜10 membered heteroaryl ring);
R^{Y2} is selected from a group consisting of hydrogen, halogen, cyano, hydroxyl, —C_{1˜6}alkyl, halo-C_{1˜6}alkyl, —O(C_{1˜6}alkyl), —O(halo-C_{1˜6}alkyl), —NH₂, —NH(C_{1˜6}alkyl), —N(C_{1˜6}alkyl) (C_{1˜6}alkyl), —C_{0˜2}alkylene-(3˜10 membered cycloalkyl), —C_{0˜2}alkylene-(3˜10 membered heterocycloalkyl), —C_{0˜2}alkylene-(5˜10 membered aryl ring), —C_{0˜2}alkylene-(5˜10 membered heteroaryl ring);
R₁ is selected from a group consisting of hydrogen, halogen, cyano, nitro, amino, hydroxyl, —C_{1˜6}alkyl, —O(C_{1˜6}alkyl), halo-C_{1˜6}alkyl, —C_{0˜2}alkylene-OR^{1b}, —C_{0˜2}alkylene-C(O)R^{1b}, —C_{0˜2}alkylene-C(O)NR^{1b}R^{1c}—C_{0˜2}alkylene-NR^{1b}R^{1c}—C_{0˜2}alkylene-NR^{1b}C(O)R^{1c}—C_{0˜4}alkylene-S(O)₂R^{1b}, —C_{0˜2}alkylene-(3˜10 membered cycloalkyl), —C_{0˜2}alkylene-(3˜10 membered heterocycloalkyl), —C_{0˜2}alkylene-(5˜10 membered aryl ring), —C_{0˜2}alkylene-(5˜10 membered heteroaryl ring); wherein said alkyl, alkylene, cycloalkyl, heterocycloalkyl, aryl ring, heteroaryl ring is optionally further substituted by one, two or three independent R^{1b};
R^{1b}, R^{1c} is each independently selected from a group consisting of hydrogen, —OH, C_{1˜6}alkyl, halo-C_{1˜6}alkyl, —O(C_{1˜6}alkyl), —O(halo-C_{1˜6}alkyl), —NH₂, —NH(C_{1˜6}alkyl), —N(C_{1˜6}alkyl) (C_{1˜6}alkyl), —C_{0˜2}alkylene-(3˜10 membered cycloalkyl), —C_{0˜2}alkylene-(3˜10 membered heterocycloalkyl), —C_{0˜2}alkylene-(5˜10 membered aryl ring), —C_{0˜2}alkylene-(5˜10 membered heteroaryl ring);
R₂ is selected from a group consisting of —C_{0˜2}alkylene-(3˜10 membered cycloalkyl), —C_{0˜2}alkylene-(3˜10 membered heterocycloalkyl); wherein said alkylene, cycloalkyl, heterocycloalkyl is optionally further substituted by one, two or three independent R^{2b};
R^{2b} is each independently selected from a group consisting of hydrogen, halogen, cyano, nitro, amino, hydroxyl, —C_{1˜6}alkyl, —O(C_{1˜6}alkyl), halo-C_{1˜6}alkyl, —C_{0˜2}alkylene-OR², —C_{0˜2}alkylene-C(O)R^{2c}, —C_{0˜2}alkylene-C(O)NR^{2c}R^{2d}, —C_{0˜2}alkylene-NR^{2c}R^{2d}, —C_{0˜2}alkylene-NR^{2c}C(O)R^{2d}, —C_{0˜4}alkylene-S(O)₂R^{2c}, —C_{0˜2}alkylene-(3˜10 membered cycloalkyl), —C_{0˜2}alkylene-(3˜10 membered heterocycloalkyl), —C_{0˜2}alkylene-(5˜10 membered aryl ring), —C_{0˜2}alkylene-(5˜10 membered heteroaryl ring); wherein alkyl, alkylene, cycloalkyl, heterocycloalkyl, aryl ring, heteroaryl ring is optionally further substituted by one, two or three independent R^{2c};
R^{2c}, R^{2d} is each independently selected from a group consisting of hydrogen, cyano, nitro, —OH, halogen, —C_{1˜6}alkyl, halo-C_{1˜6}alkyl, —C_{0˜2}alkylene-O(C_{1˜6}alkyl), —O(halo-C_{1˜6}alkyl), —O(3˜10 membered cycloalkyl), —O(3˜10 membered heterocycloalkyl), —NH₂, —NH(C_{1˜6}alkyl), —N(C_{1˜6}alkyl) (C_{1˜6}alkyl), —C_{0˜2}alkylene-(3˜10 membered cycloalkyl), —C_{0˜2}alkylene-(3˜10 membered heterocycloalkyl), —C_{0˜2}alkylene-(5˜10 membered aryl ring), —C_{0˜2}alkylene-(5˜10 membered heteroaryl ring); wherein said alkyl, alkylene, cycloalkyl, heterocycloalkyl, aryl ring, heteroaryl ring is optionally further substituted by one, two or three independent R^{2e};
R^{2e} is each independently selected from a group consisting of hydrogen, cyano, nitro, —OH, halogen, —C_{1˜6}alkyl, halo-C_{1˜6}alkyl, —C_{0˜2}alkylene-O(C_{1˜6}alkyl), —O(halo-C_{1˜6}alkyl), —O(3˜10 membered cycloalkyl), —O(3˜10 membered heterocycloalkyl), —NH₂, —NH(C_{1˜6}alkyl), —N(C_{1˜6}alkyl) (C_{1˜6}alkyl), —C_{0˜2}alkylene-(3˜10 membered cycloalkyl), —C_{0˜2}alkylene-(3˜10 membered heterocycloalkyl), —C_{0˜2}alkylene-(5˜10 membered aryl ring), —C_{0˜2}alkylene-(5˜10 membered heteroaryl ring);
R₃ is selected from a group consisting of hydrogen, halogen, cyano, nitro, amino, —OH, —C_{1˜6}alkyl, —O(C_{1˜6}alkyl), halo-C_{1˜6}alkyl, —C_{0˜2}alkylene-OR^{3b}, —C_{0˜2}alkylene-C(O)R^{3b}, —C_{0˜2}alkylene-C(O)NR^{3b}R^{3c}, —C_{0˜2}alkylene-NR^{3b}R^{3c}, —C_{0˜2}alkylene-NR^{3b}C(O)R^{3c}, —C_{0˜4}alkylene-S(O)₂R^{3b}, —C_{0˜2}alkylene-(3˜10 membered cycloalkyl), —C_{0˜2}alkylene-(3˜10 membered heterocycloalkyl);
R^{3b}, R^{3c} is each independently selected from a group consisting of hydrogen, halogen, cyano, nitro, —OH, C_{1˜6}alkyl, halo-C_{1˜6}alkyl, —O(C_{1˜6}alkyl), —O(halo-C_{1˜6}alkyl), —NH₂, —NH(C_{1˜6}alkyl), —N(C_{1˜6}alkyl) (C_{1˜6}alkyl), —C_{0˜2}alkylene-(3˜10 membered cycloalkyl), —C_{0˜2}alkylene-(3˜10 membered heterocycloalkyl), —C_{0˜2}alkylene-(5˜10 membered aryl ring), —C_{0˜2}alkylene-(5˜10 membered heteroaryl ring);
R₄ is selected from a group consisting of hydrogen, halogen, cyano, nitro, hydroxyl, —C_{1˜6}alkyl, halo-C_{1˜6}alkyl, —O(C_{1˜6}alkyl), —O(halo-C_{1˜6}alkyl), —NH₂, —NH(C_{1˜6}alkyl), —N(C_{1˜6}alkyl) (C_{1˜6}alkyl), —C_{0˜2}alkylene-(3˜10 membered cycloalkyl), —C_{0˜2}alkylene-(3˜10 membered heterocycloalkyl), —C_{0˜2}alkylene-(5˜10 membered aryl ring), —C_{0˜2}alkylene-(5˜10 membered heteroaryl ring); wherein said alkyl, alkylene, cycloalkyl, heterocycloalkyl, aryl ring, heteroaryl ring is optionally further substituted by one, two or three independent R^{4b};
R^{4b} is each independently selected from a group consisting of hydrogen, halogen, cyano, nitro, —OH, C_{1˜6}alkyl, halo-C_{1˜6}alkyl, —O(C_{1˜6}alkyl), —O(halo-C_{1˜6}alkyl), —NH₂, —NH(C_{1˜6}alkyl), —N(C_{1˜6}alkyl) (C_{1˜6}alkyl), —C_{0˜2}alkylene-(3˜10 membered cycloalkyl), —C_{0˜2}alkylene-(3˜10 membered heterocycloalkyl), —C_{0˜2}alkylene-(5˜10 membered aryl ring), —C_{0˜2}alkylene-(5˜10 membered heteroaryl ring);
R₅ is selected from a group consisting of hydrogen, halogen, cyano, nitro, hydroxyl, —C_{1˜6}alkyl, halo-C_{1˜6}alkyl, —O(C_{1˜6}alkyl), —O(halo-C_{1˜6}alkyl), —NH₂, —NH(C_{1˜6}alkyl), —N(C_{1˜6}alkyl) (C_{1˜6}alkyl), —C_{0˜2}alkylene-(3˜10 membered cycloalkyl), —C_{0˜2}alkylene-(3˜10 membered heterocycloalkyl), —C_{0˜2}alkylene-(5˜10 membered aryl ring), —C_{0˜2}alkylene-(5˜10 membered heteroaryl ring);
R₆ is selected from a group consisting of hydrogen, halogen, cyano, nitro, hydroxyl, —C_{1˜6}alkyl, halo-C_{1˜6}alkyl, —O(C_{1˜6}alkyl), —O(halo-C_{1˜6}alkyl), —NH₂, —NH(C_{1˜6}alkyl), —N(C_{1˜6}alkyl) (C_{1˜6}alkyl), —C_{0˜2}alkylene-(3˜10 membered cycloalkyl), —C_{0˜2}alkylene-(3˜10 membered heterocycloalkyl), —C_{0˜2}alkylene-(5˜10 membered aryl ring), —C_{0˜2}alkylene-(5˜10 membered heteroaryl ring).

13. The compound, or a deuterated compound thereof, or a stereoisomer thereof, or a pharmaceutically acceptable salt thereof according to claim 12, wherein said R₁, R₃, R₄, R₅, R₆ is each independently selected from a group consisting of hydrogen, halogen, —C_{1˜3}alkyl;
Q is selected from a group consisting of single bond; Y is selected from a group consisting of CHR^{Y2}; R^{Y2} is selected from a group consisting of hydrogen, —C_{1˜3}alkyl.

14. The compound, or a deuterated compound thereof, or a stereoisomer thereof, or a pharmaceutically acceptable salt thereof according to claim 12, wherein said R₂ is specifically selected from a group consisting of 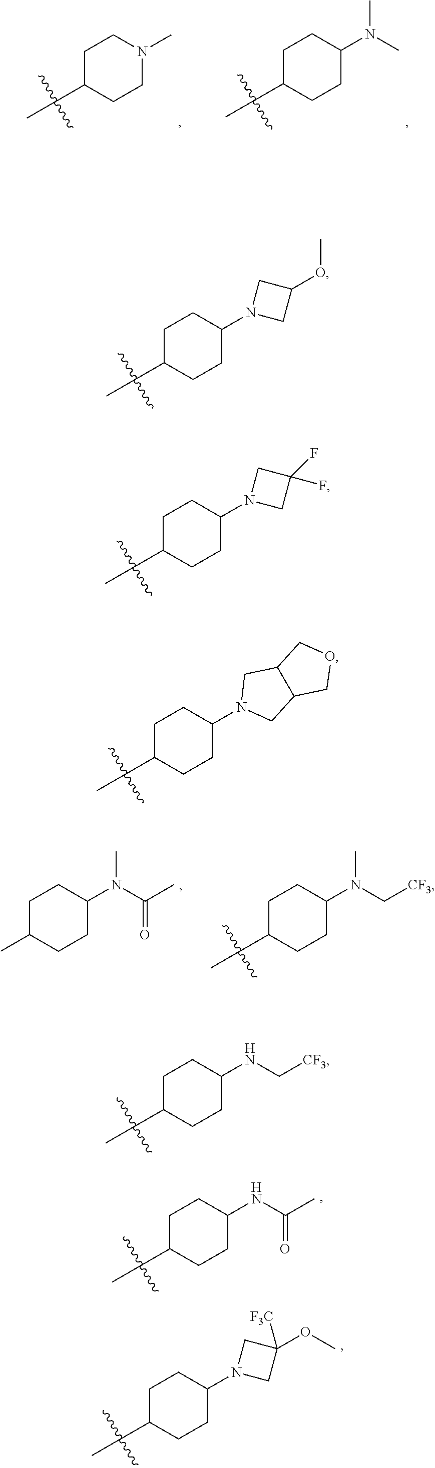 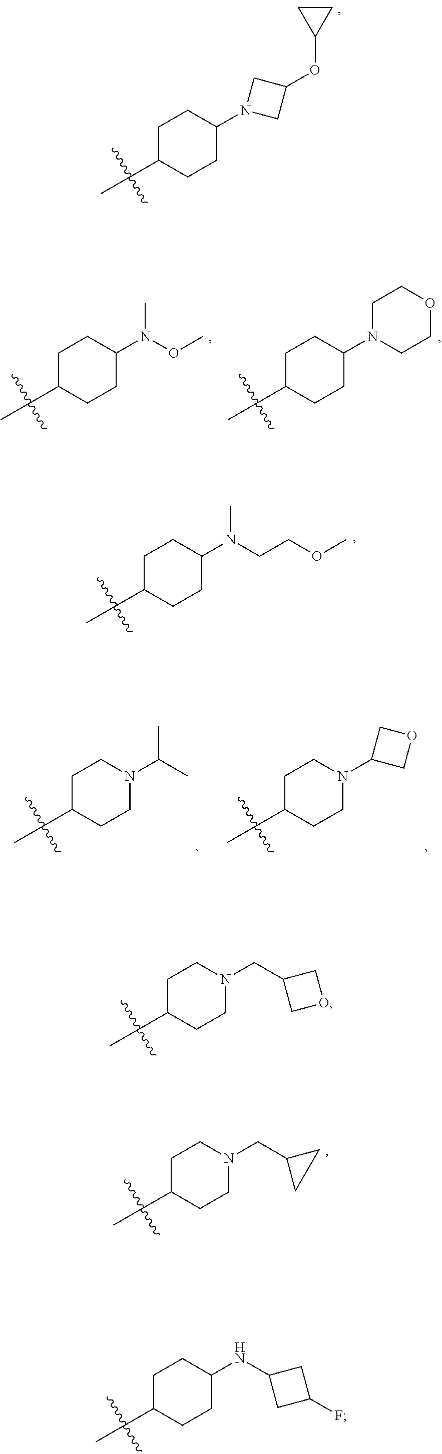
preferably, said R₂ is specifically selected from a group consisting of

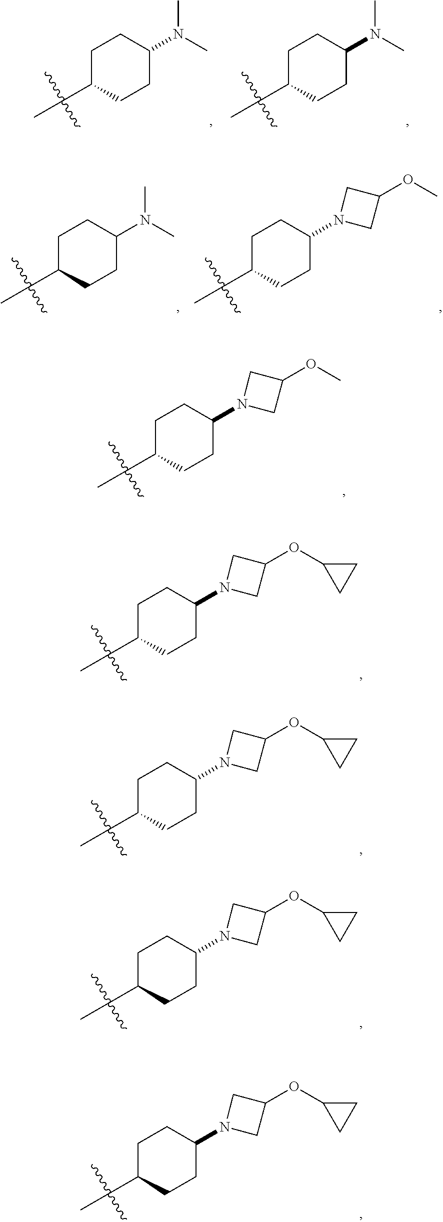

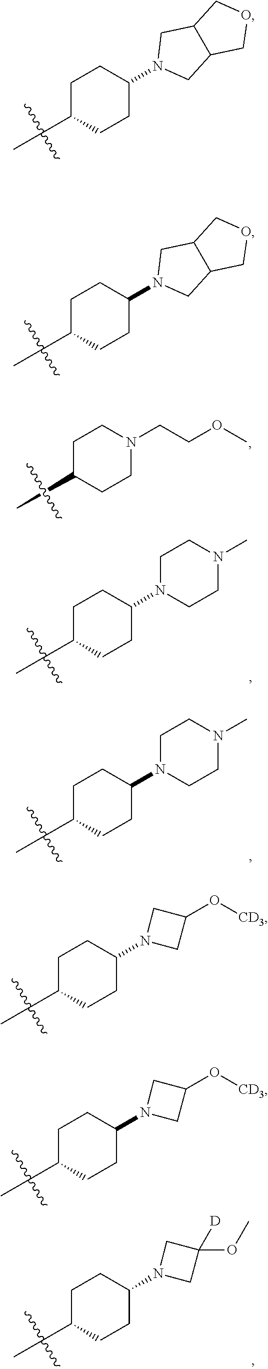

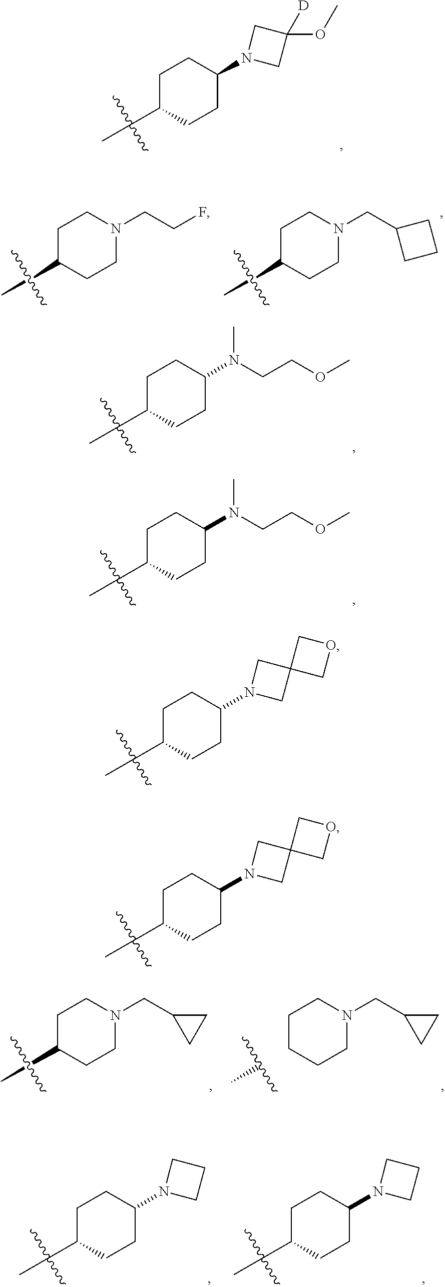

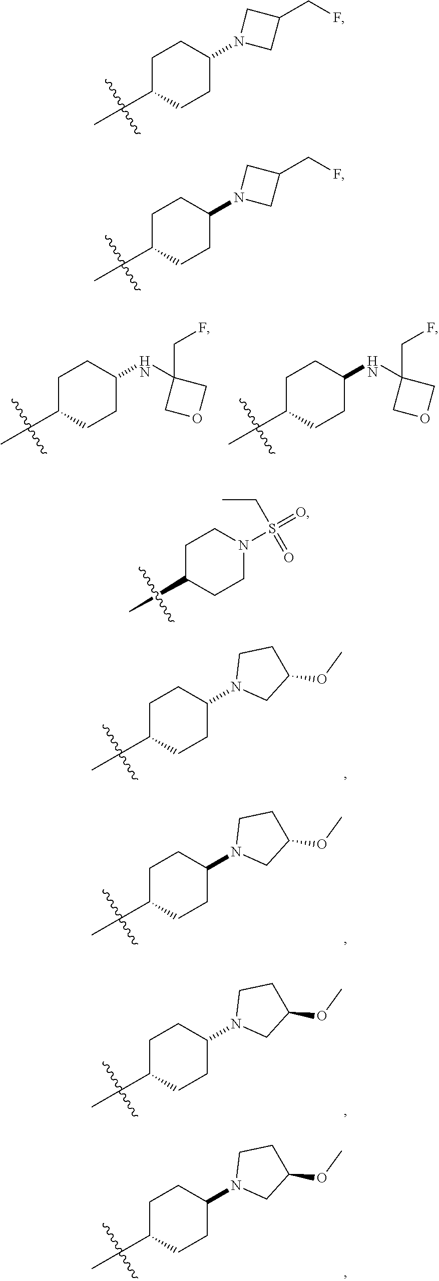

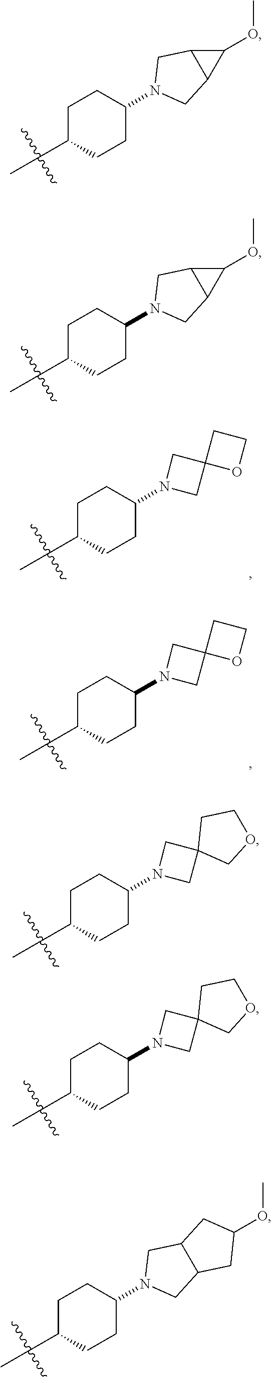

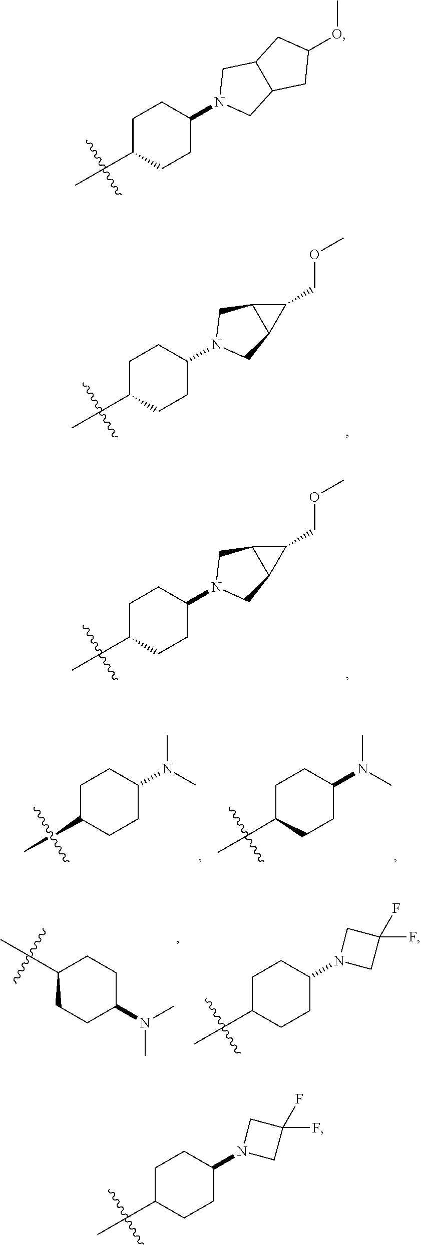

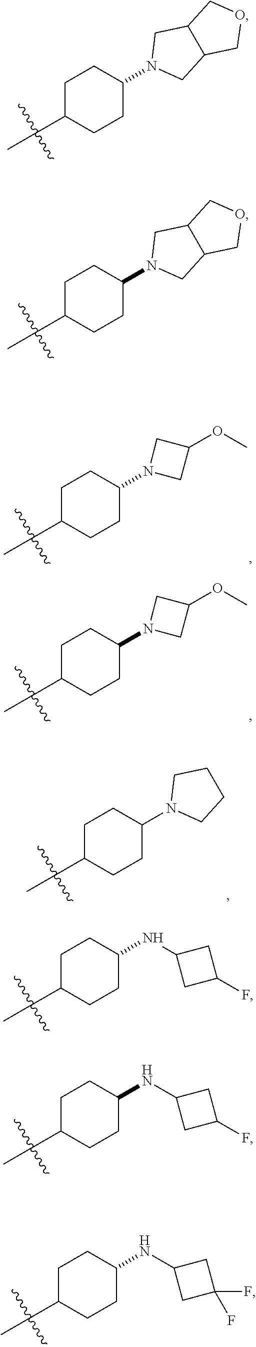

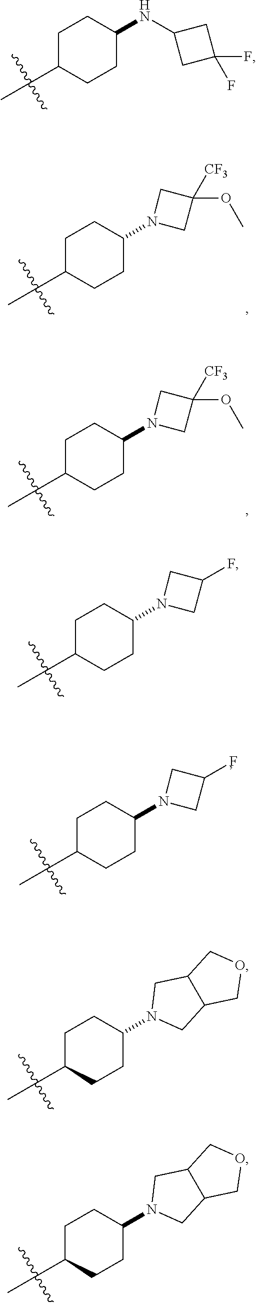

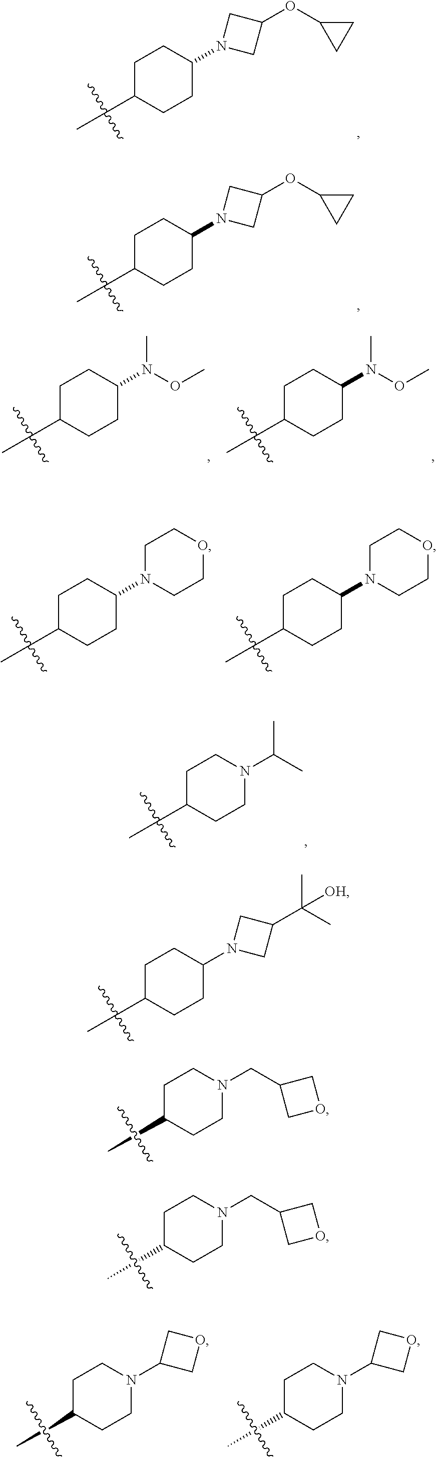

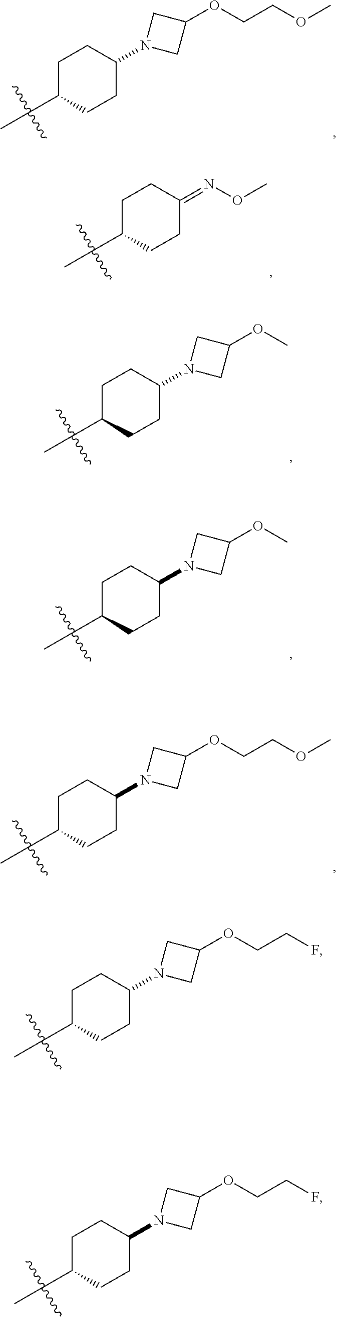

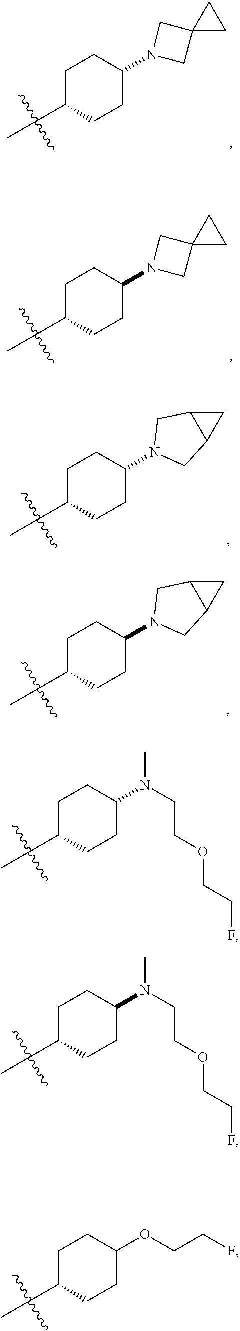

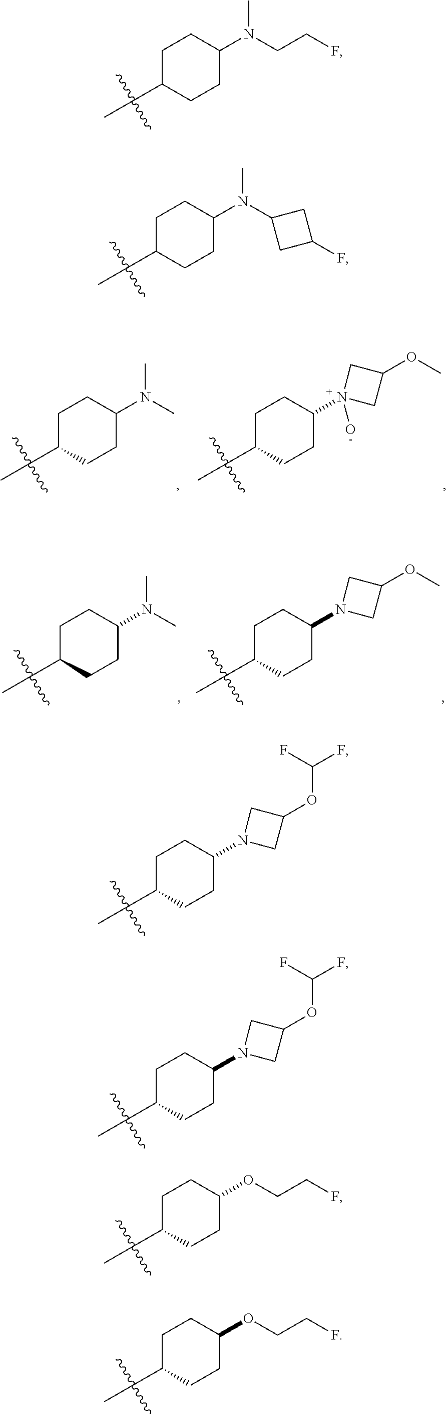

15. The compound, or a deuterated compound thereof, or a stereoisomer thereof, or a pharmaceutically acceptable salt thereof according to claim 1, including the following compounds represented by Formula IIc: 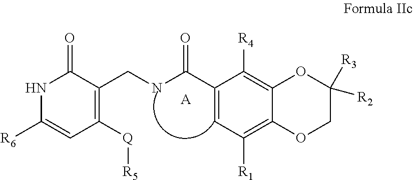
Q is selected from a group consisting of single bond, O or S;
A-ring is selected from a group consisting of 4-10 membered heterocyclic ring; wherein said heterocyclic ring is optionally further substituted by one, two or three independent R⁷¹;
each R⁷¹ is each independently selected from a group consisting of hydrogen, halogen, cyano, nitro, —OH, C_{1˜6}alkyl, halo-C_{1˜6}alkyl, —O(C_{1˜6}alkyl), —O(halo-C_{1˜6}alkyl), —NH₂, —NH(C_{1˜6}alkyl), —N(C_{1˜6}alkyl) (C_{1˜6}alkyl), —C_{0˜2}alkylene-(3˜10 membered cycloalkyl), —C_{0˜2}alkylene-(3˜10 membered heterocycloalkyl), —C_{0˜2}alkylene-(5˜10 membered aryl ring), —C_{0˜2}alkylene-(5˜10 membered heteroaryl ring);
R¹ is selected from a group consisting of hydrogen, halogen, cyano, nitro, amino, hydroxyl, —C_{1˜6}alkyl, —O(C_{1˜6}alkyl), halo-C_{1˜6}alkyl, —C_{0˜2}alkylene-OR^{1b}, —C_{0˜2}alkylene-C(O)R^{1b}, —C_{0˜2}alkylene-C(O)NR^{1b}R^{1c}, —C_{0˜2}alkylene-NR^{1b}R^{1c}, —C_{0˜2}alkylene-NR^{1b}C(O)R^{1c}, —C_{0˜4}alkylene-S(O)₂R^{1b}R^{1c}, —C_{0˜2}alkylene-(3˜10 membered cycloalkyl), —C_{0˜2}alkylene-(3˜10 membered heterocycloalkyl), —C_{0˜2}alkylene-(5˜10 membered aryl ring), —C_{0˜2}alkylene-(5˜10 membered heteroaryl ring); wherein said alkyl, alkylene, cycloalkyl, heterocycloalkyl, aryl ring, heteroaryl ring is optionally further substituted by one, two or three independent R^{1b};
R^{1b}, R^{1c} is each independently selected from a group consisting of hydrogen, —OH, C_{1˜6}alkyl, halo-C_{1˜6}alkyl, —O(C_{1˜6}alkyl), —O(halo-C_{1˜6}alkyl), —NH₂, —NH(C_{1˜6}alkyl), —N(C_{1˜6}alkyl) (C_{1˜6}alkyl), —C_{0˜2}alkylene-(3˜10 membered cycloalkyl), —C_{0˜2}alkylene-(3˜10 membered heterocycloalkyl), —C_{0˜2}alkylene-(5˜10 membered aryl ring), —C_{0˜2}alkylene-(5˜10 membered heteroaryl ring);
R₂ is selected from a group consisting of —C_{0˜2}alkylene-(3˜10 membered cycloalkyl), —C_{0˜2}alkylene-(3˜10 membered heterocycloalkyl); wherein said alkylene, cycloalkyl, heterocycloalkyl is optionally further substituted by one, two or three independent R^{2b};
R^{2b} is each independently selected from a group consisting of hydrogen, halogen, cyano, nitro, amino, hydroxyl, —C_{1˜6}alkyl, —O(C_{1˜6}alkyl), halo-C_{1˜6}alkyl, —C_{0˜2}alkylene-OR², —C_{0˜2}alkylene-C(O)R^{2c}, —C_{0˜2}alkylene-C(O)NR^{2c}R^{2d}, —C_{0˜2}alkylene-NR^{2c}R^{2d}, —C_{0˜2}alkylene-NR^{2c}C(O)R^{2d}, —C_{0˜4}alkylene-S(O)₂R^{2c}, —C_{0˜2}alkylene-(3˜10 membered cycloalkyl), —C_{0˜2}alkylene-(3˜10 membered heterocycloalkyl), —C_{0˜2}alkylene-(5˜10 membered aryl ring), —C_{0˜2}alkylene-(5˜10 membered heteroaryl ring); wherein alkyl, alkylene, cycloalkyl, heterocycloalkyl, aryl ring, heteroaryl ring is optionally further substituted by one, two or three independent R^{2e};
R^{2c}, R^{2d} is each independently selected from a group consisting of hydrogen, cyano, nitro, —OH, halogen, —C_{1˜6}alkyl, halo-C_{1˜6}alkyl, —C_{0˜2}alkylene-O(C_{1˜6}alkyl), —O(halo-C_{1˜6}alkyl), —O(3˜10 membered cycloalkyl), —O(3˜10 membered heterocycloalkyl), —NH₂, —NH(C_{1˜6}alkyl), —N(C_{1˜6}alkyl) (C_{1˜6}alkyl), —C_{0˜2}alkylene-(3˜10 membered cycloalkyl), —C_{0˜2}alkylene-(3˜10 membered heterocycloalkyl), —C_{0˜2}alkylene-(5˜10 membered aryl ring), —C_{0˜2}alkylene-(5˜10 membered heteroaryl ring); wherein said alkyl, alkylene, cycloalkyl, heterocycloalkyl, aryl ring, heteroaryl ring is optionally further substituted by one, two or three independent R^{2e};
R^{2e} is each independently selected from a group consisting of hydrogen, cyano, nitro, —OH, halogen, —C_{1˜6}alkyl, halo-C_{1˜6}alkyl, —C_{0˜2}alkylene-O(C_{1˜6}alkyl), —O(halo-C_{1˜6}alkyl), —O(3˜10 membered cycloalkyl), —O(3˜10 membered heterocycloalkyl), —NH₂, —NH(C_{1˜6}alkyl), —N(C_{1˜6}alkyl) (C_{1˜6}alkyl), —C_{0˜2}alkylene-(3˜10 membered cycloalkyl), —C_{0˜2}alkylene-(3˜10 membered heterocycloalkyl), —C_{0˜2}alkylene-(5˜10 membered aryl ring), —C_{0˜2}alkylene-(5˜10 membered heteroaryl ring);
R₃ is selected from a group consisting of hydrogen, halogen, cyano, nitro, amino, —OH, —C_{1˜6}alkyl, —O(C_{1˜6}alkyl), halo-C_{1˜6}alkyl, —C_{0˜2}alkylene-OR^{3b}, —C_{0˜2}alkylene-C(O)R^{3b}, —C_{0˜2}alkylene-C(O)NR^{3b}R^{3c}, —C_{0˜2}alkylene-NR^{3b}R^{3c}, —C_{0˜2}alkylene-NR^{3b}C(O)R^{3c}, —C_{0˜4}alkylene-S(O)₂R^{3b}, —C_{0˜2}alkylene-(3˜10 membered cycloalkyl), —C_{0˜2}alkylene-(3˜10 membered heterocycloalkyl);
R^{3b}, R^{3c} is each independently selected from a group consisting of hydrogen, halogen, cyano, nitro, —OH, C_{1˜6}alkyl, halo-C_{1˜6}alkyl, —O(C_{1˜6}alkyl), —O(halo-C_{1˜6}alkyl), —NH₂, —NH(C_{1˜6}alkyl), —N(C_{1˜6}alkyl) (C_{1˜6}alkyl), —C_{0˜2}alkylene-(3˜10 membered cycloalkyl), —C_{0˜2}alkylene-(3˜10 membered heterocycloalkyl), —C_{0˜2}alkylene-(5˜10 membered aryl ring), —C_{0˜2}alkylene-(5˜10 membered heteroaryl ring);
R₄ is selected from a group consisting of hydrogen, halogen, cyano, nitro, hydroxyl, —C_{1˜6}alkyl, halo-C_{1˜6}alkyl, —O(C_{1˜6}alkyl), —O(halo-C_{1˜6}alkyl), —NH₂, —NH(C_{1˜6}alkyl), —N(C_{1˜6}alkyl) (C_{1˜6}alkyl), —C_{0˜2}alkylene-(3˜10 membered cycloalkyl), —C_{0˜2}alkylene-(3˜10 membered heterocycloalkyl), —C_{0˜2}alkylene-(5˜10 membered aryl ring), —C_{0˜2}alkylene-(5˜10 membered heteroaryl ring); wherein said alkyl, alkylene, cycloalkyl, heterocycloalkyl, aryl ring, heteroaryl ring is optionally further substituted by one, two or three independent R^{4b};
R^{4b} is each independently selected from a group consisting of hydrogen, halogen, cyano, nitro, —OH, C_{1˜6}alkyl, halo-C_{1˜6}alkyl, —O(C_{1˜6}alkyl), —O(halo-C_{1˜6}alkyl), —NH₂, —NH(C_{1˜6}alkyl), —N(C_{1˜6}alkyl) (C_{1˜6}alkyl), —C_{0˜2}alkylene-(3˜10 membered cycloalkyl), —C_{0˜2}alkylene-(3˜10 membered heterocycloalkyl), —C_{0˜2}alkylene-(5˜10 membered aryl ring), —C_{0˜2}alkylene-(5˜10 membered heteroaryl ring);
R₅ is selected from a group consisting of hydrogen, halogen, cyano, nitro, hydroxyl, —C_{1˜6}alkyl, halo-C_{1˜6}alkyl, —O(C_{1˜6}alkyl), —O(halo-C_{1˜6}alkyl), —NH₂, —NH(C_{1˜6}alkyl), —N(C_{1˜6}alkyl) (C_{1˜6}alkyl), —C_{0˜2}alkylene-(3˜10 membered cycloalkyl), —C_{0˜2}alkylene-(3˜10 membered heterocycloalkyl), —C_{0˜2}alkylene-(5˜10 membered aryl ring), —C_{0˜2}alkylene-(5˜10 membered heteroaryl ring);
R₆ is selected from a group consisting of hydrogen, halogen, cyano, nitro, hydroxyl, —C_{1˜6}alkyl, halo-C_{1˜6}alkyl, —O(C_{1˜6}alkyl), —O(halo-C_{1˜6}alkyl), —NH₂, —NH(C_{1˜6}alkyl), —N(C_{1˜6}alkyl) (C_{1˜6}alkyl), —C_{0˜2}alkylene-(3˜10 membered cycloalkyl), —C_{0˜2}alkylene-(3˜10 membered heterocycloalkyl), —C_{0˜2}alkylene-(5˜10 membered aryl ring), —C_{0˜2}alkylene-(5˜10 membered heteroaryl ring).

16. The compound, or a deuterated compound thereof, or a stereoisomer thereof, or a pharmaceutically acceptable salt thereof according to claim 15, wherein said R₁, R₃, R₄, R₅, R₆ is each independently selected from a group consisting of hydrogen, halogen, —C_{1˜3}alkyl; Q is selected from a group consisting of single bond.

17. The compound, or a deuterated compound thereof, or a stereoisomer thereof, or a pharmaceutically acceptable salt thereof according to claim 15, wherein said R₂ is specifically selected from a group consisting of 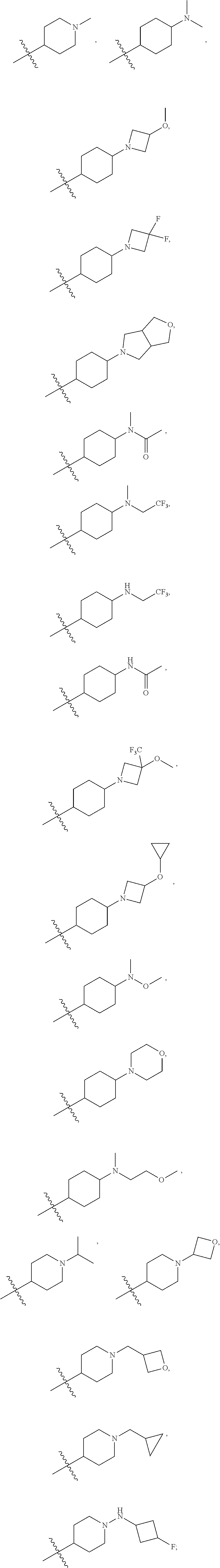
preferably, said R₂ is specifically selected from a group consisting of

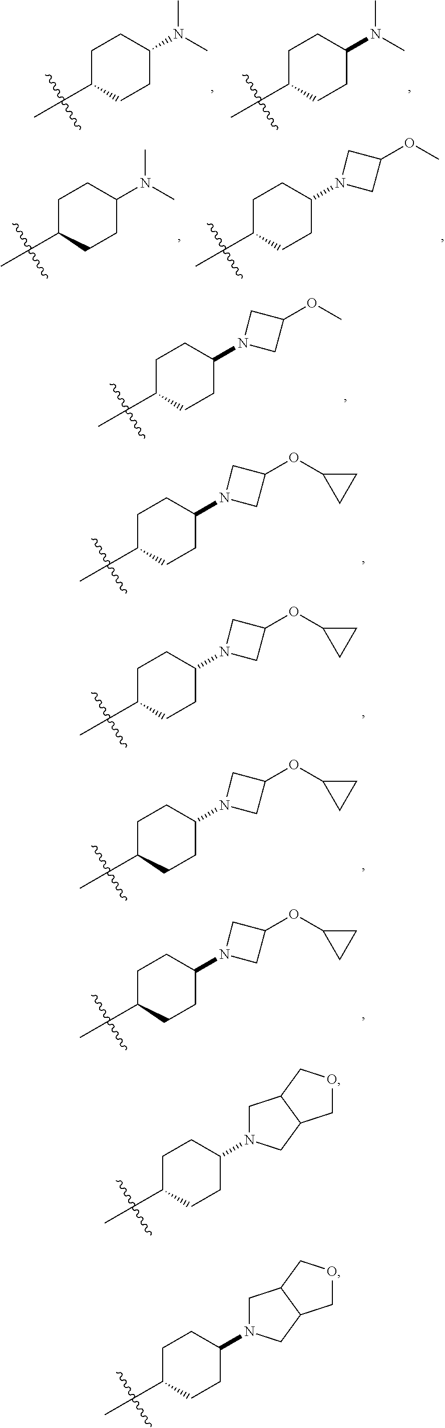

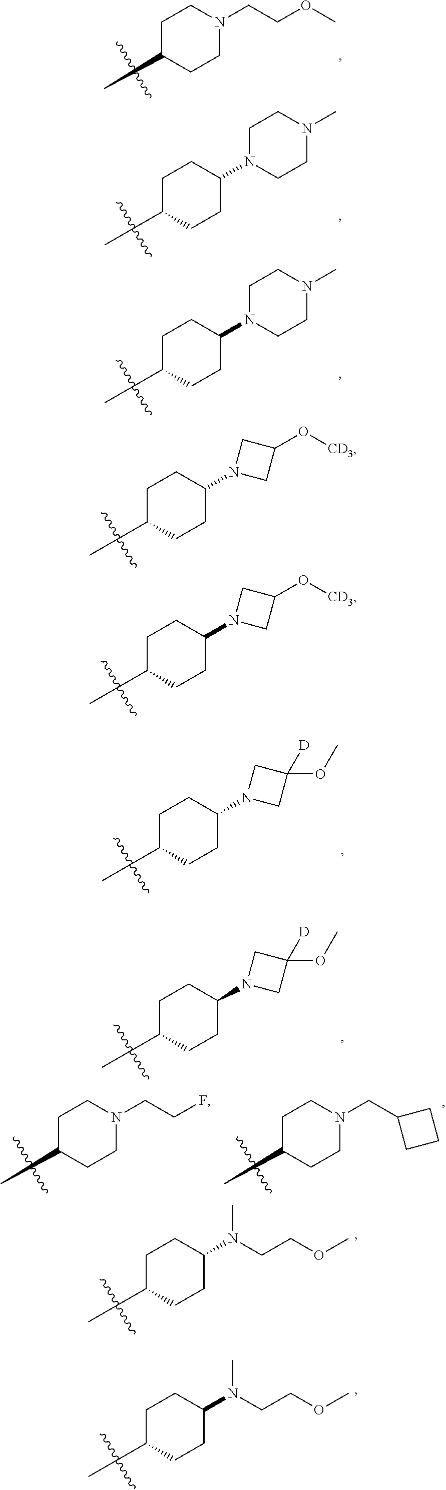

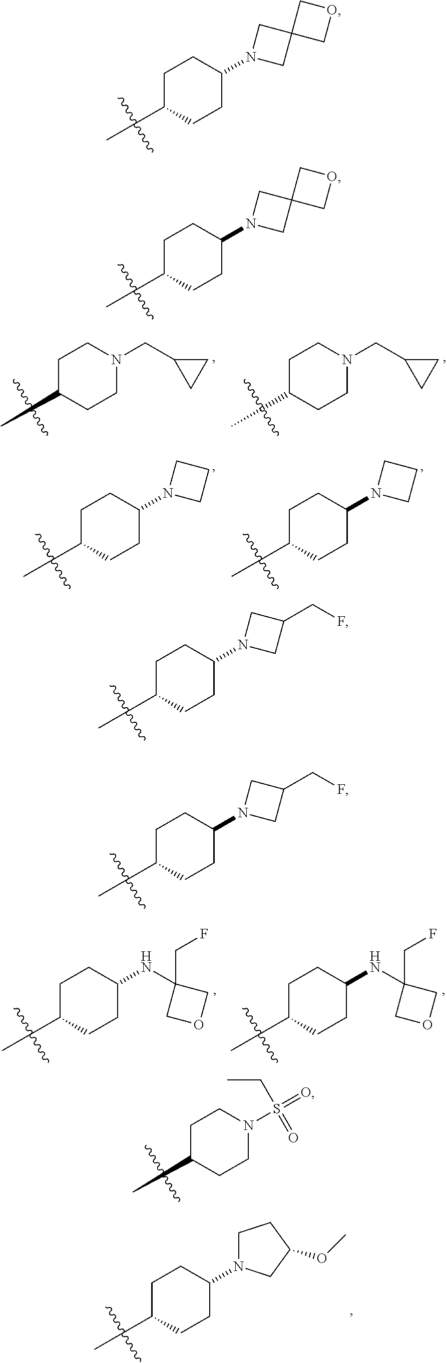

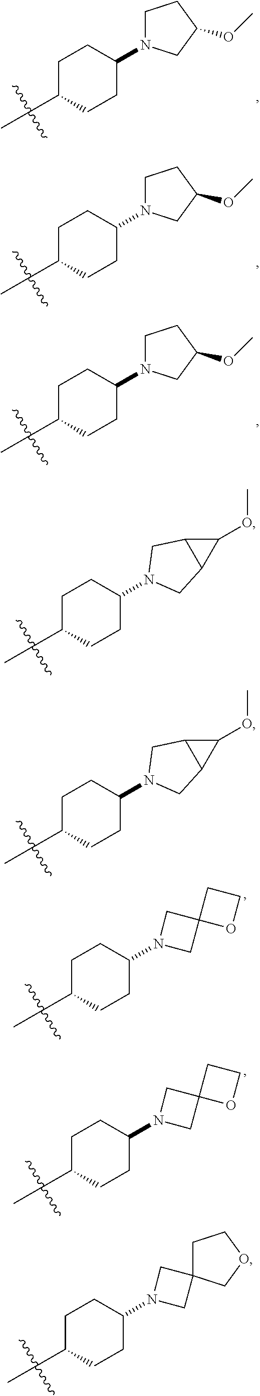

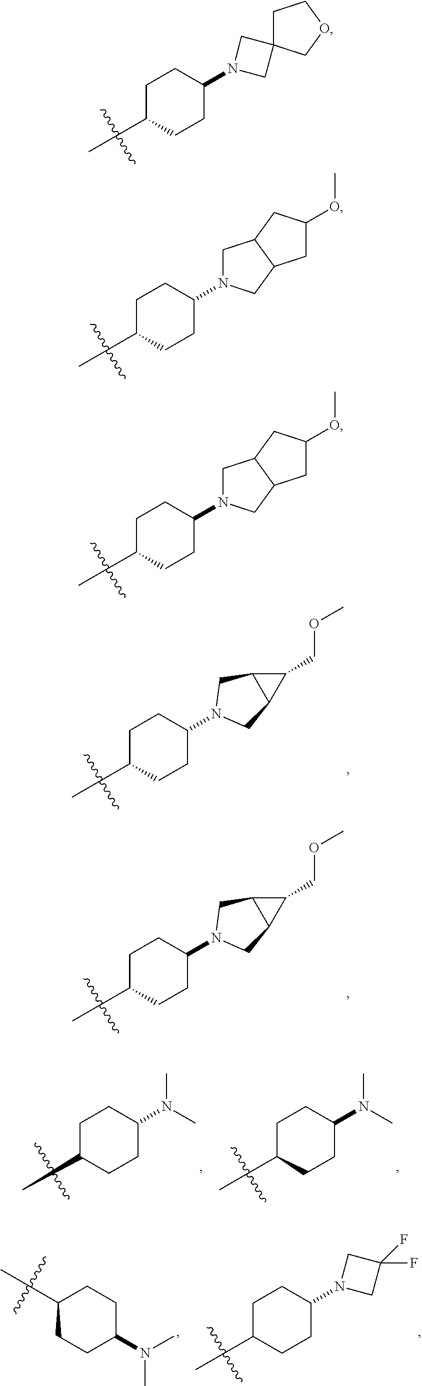

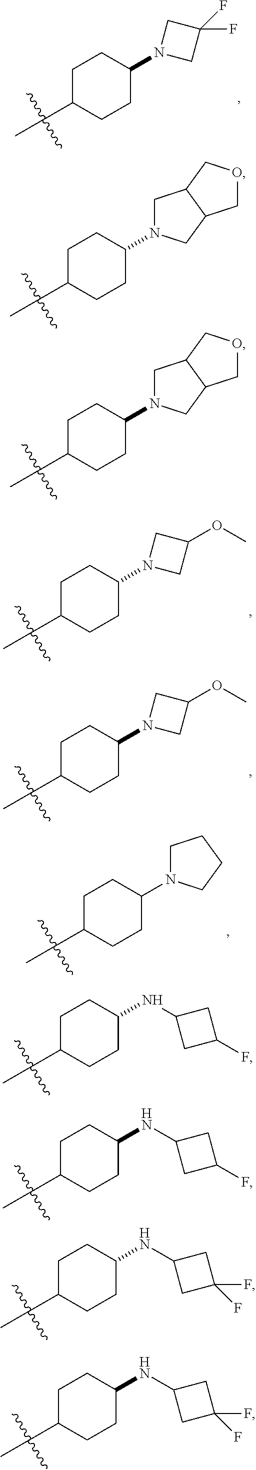

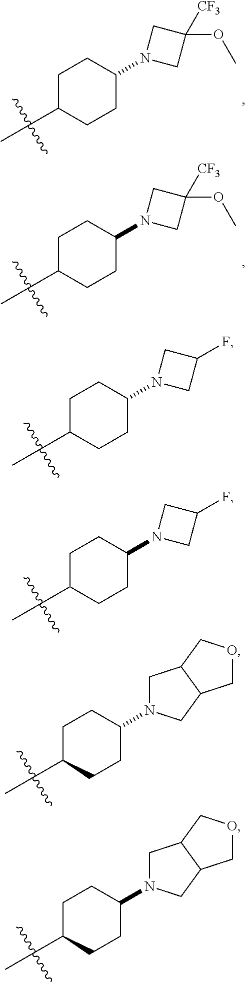

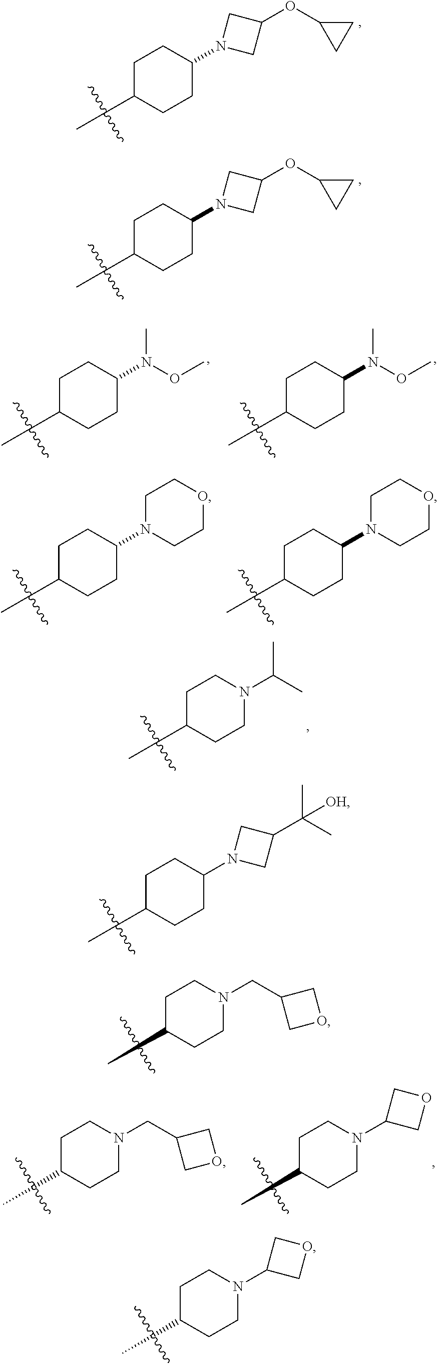

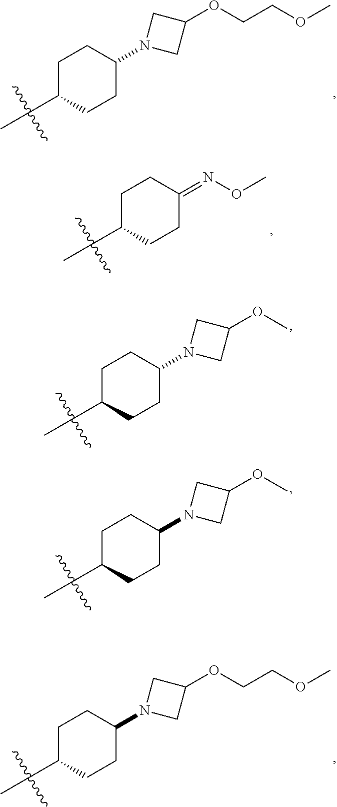

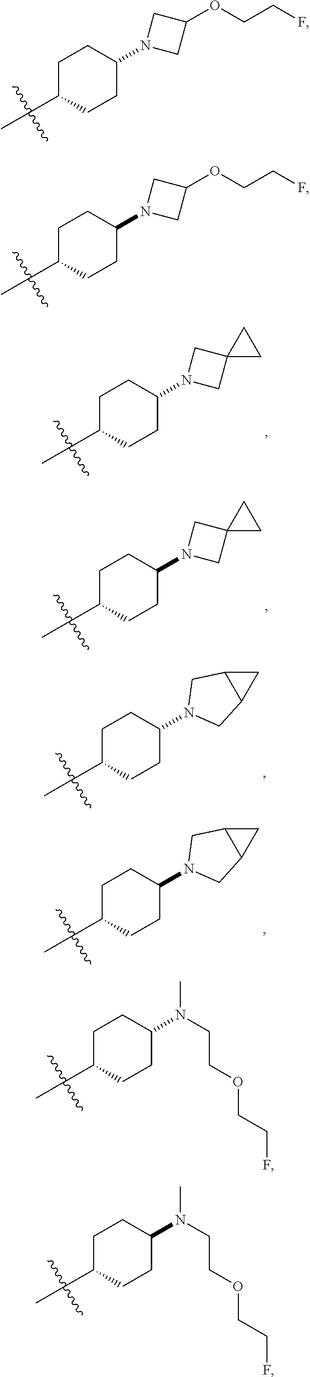

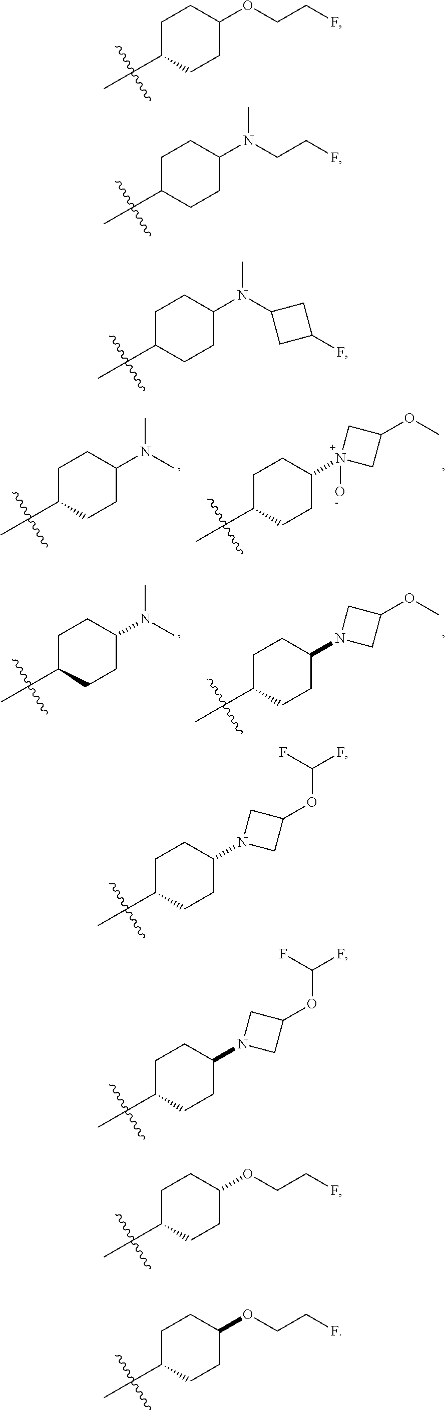

18. The compound, or a deuterated compound thereof, or a stereoisomer thereof, or a pharmaceutically acceptable salt thereof according to claim 1, wherein:
the compound represented by Formula I or Formula II is specifically:

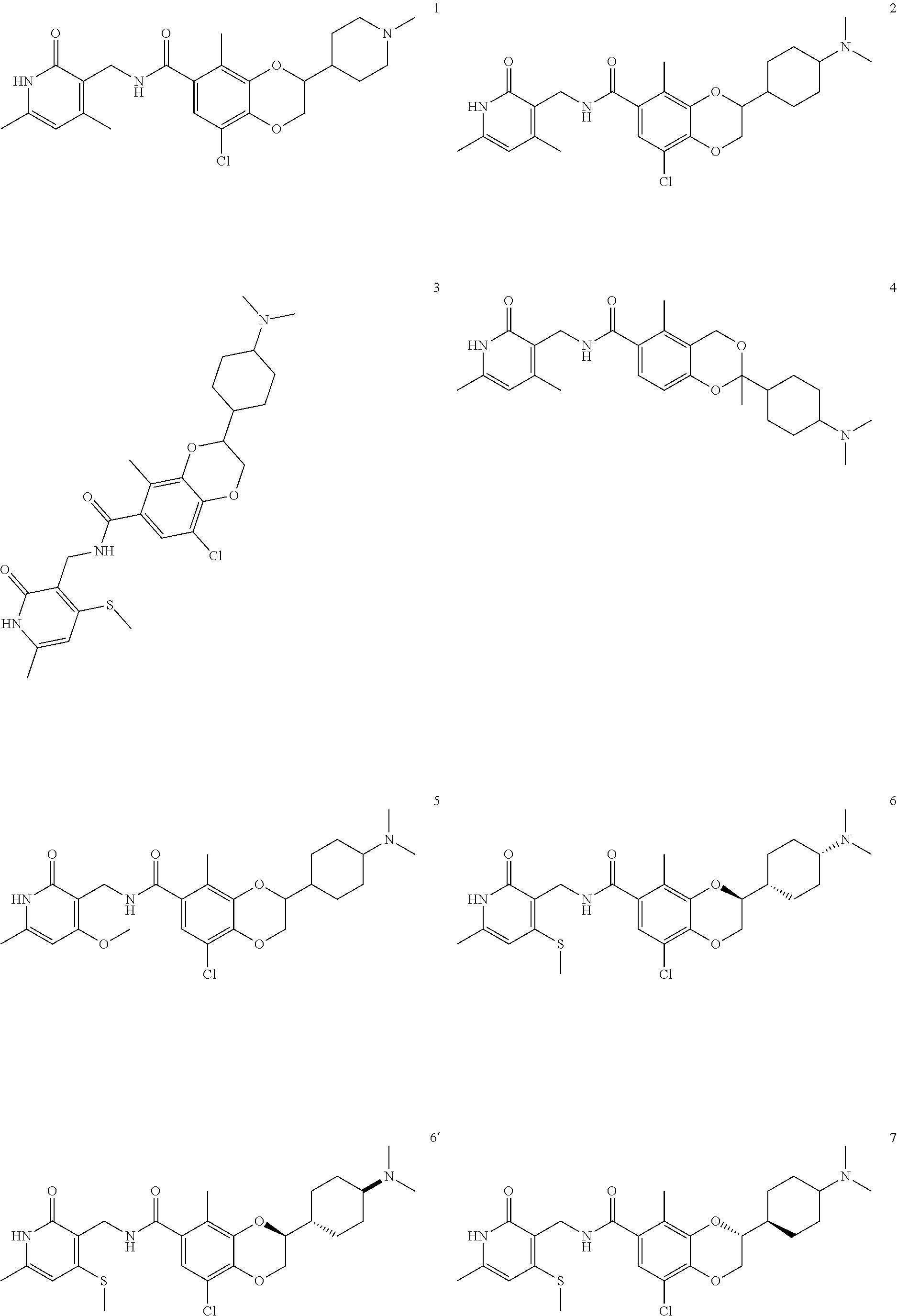

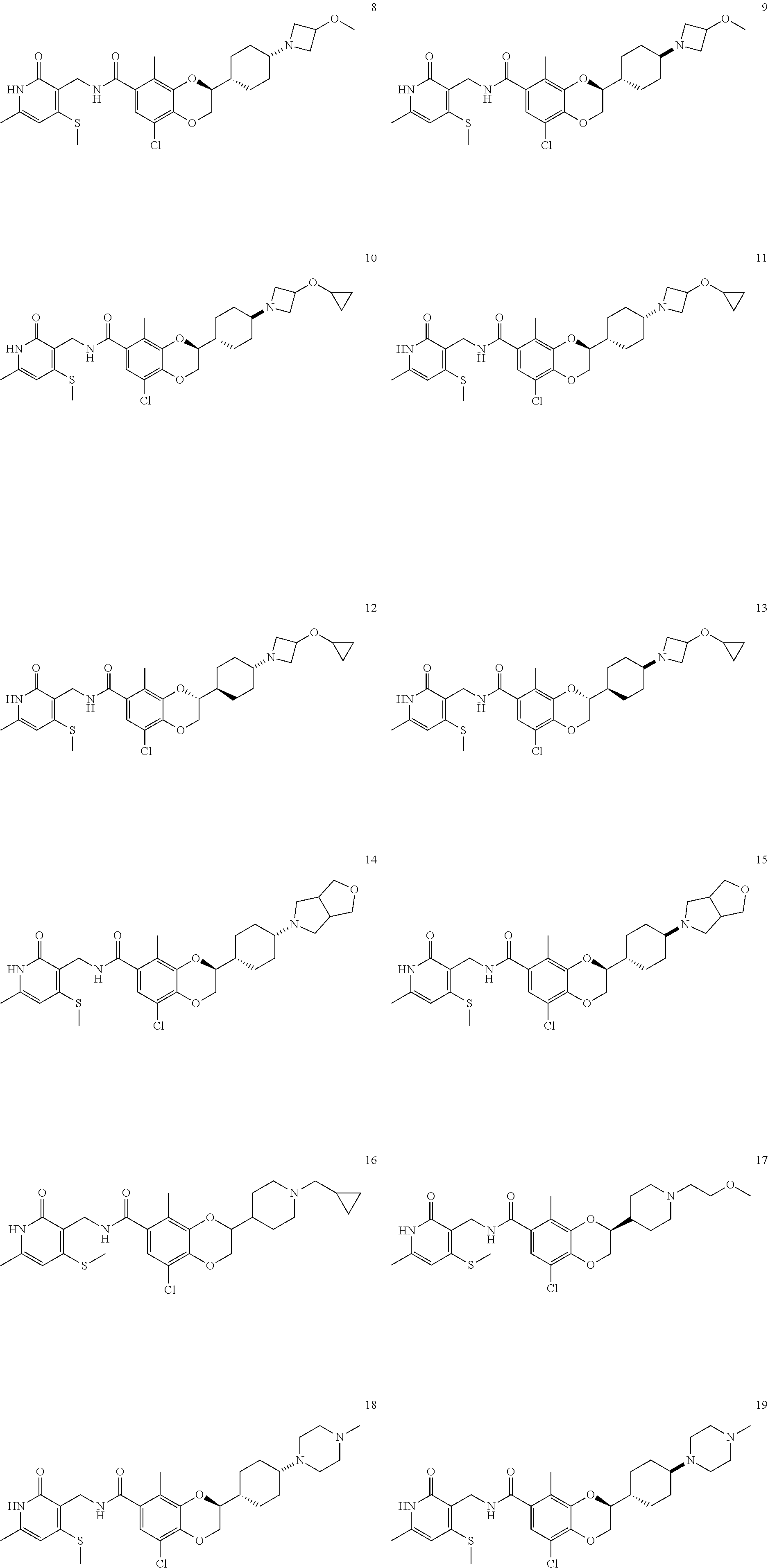

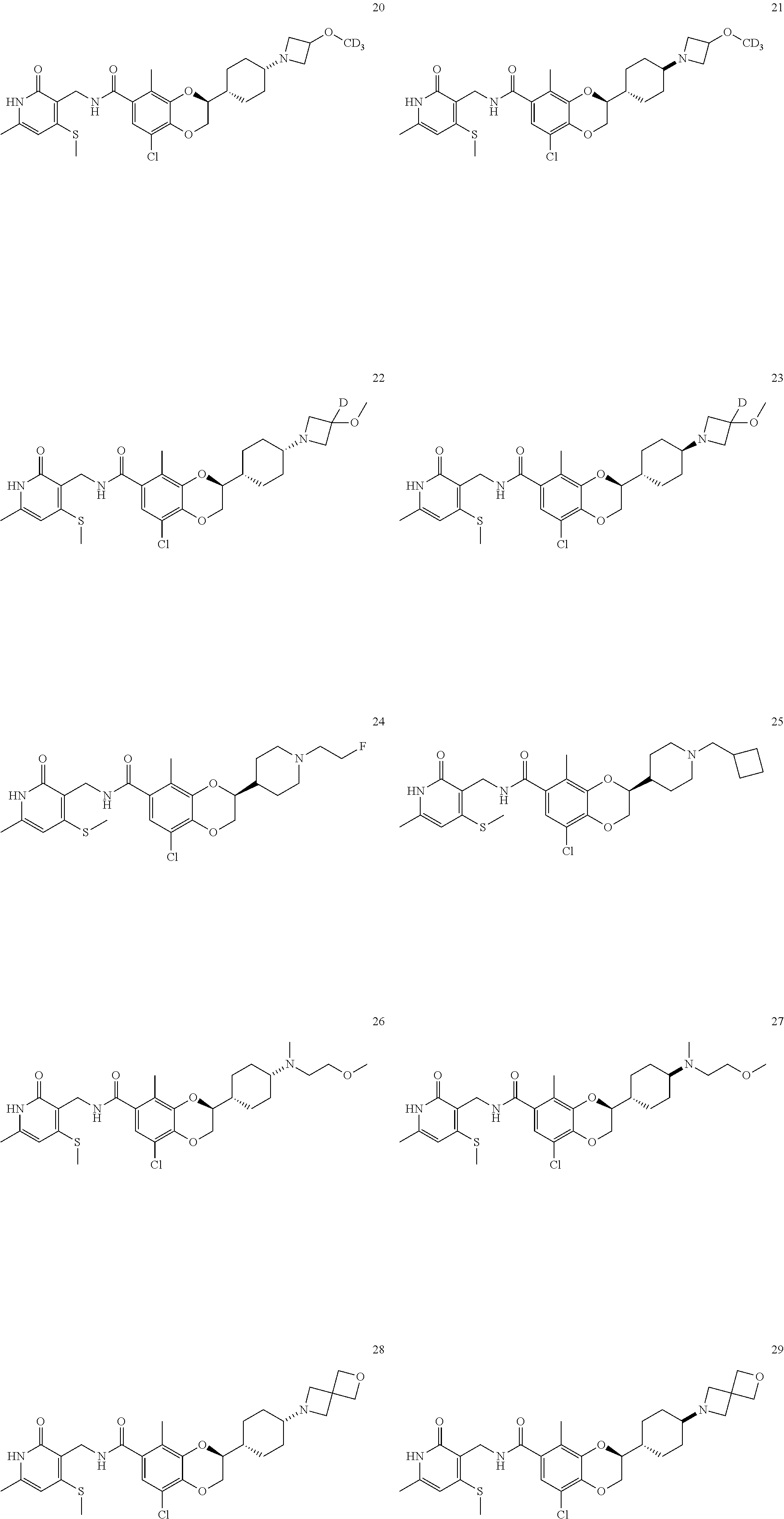

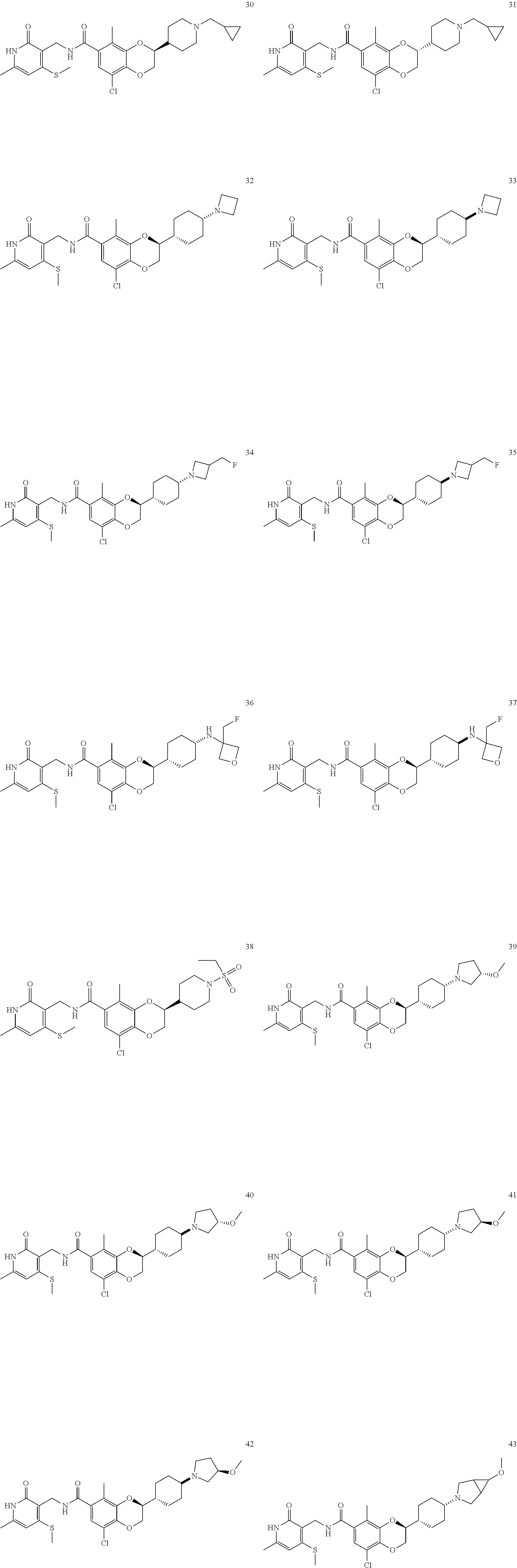

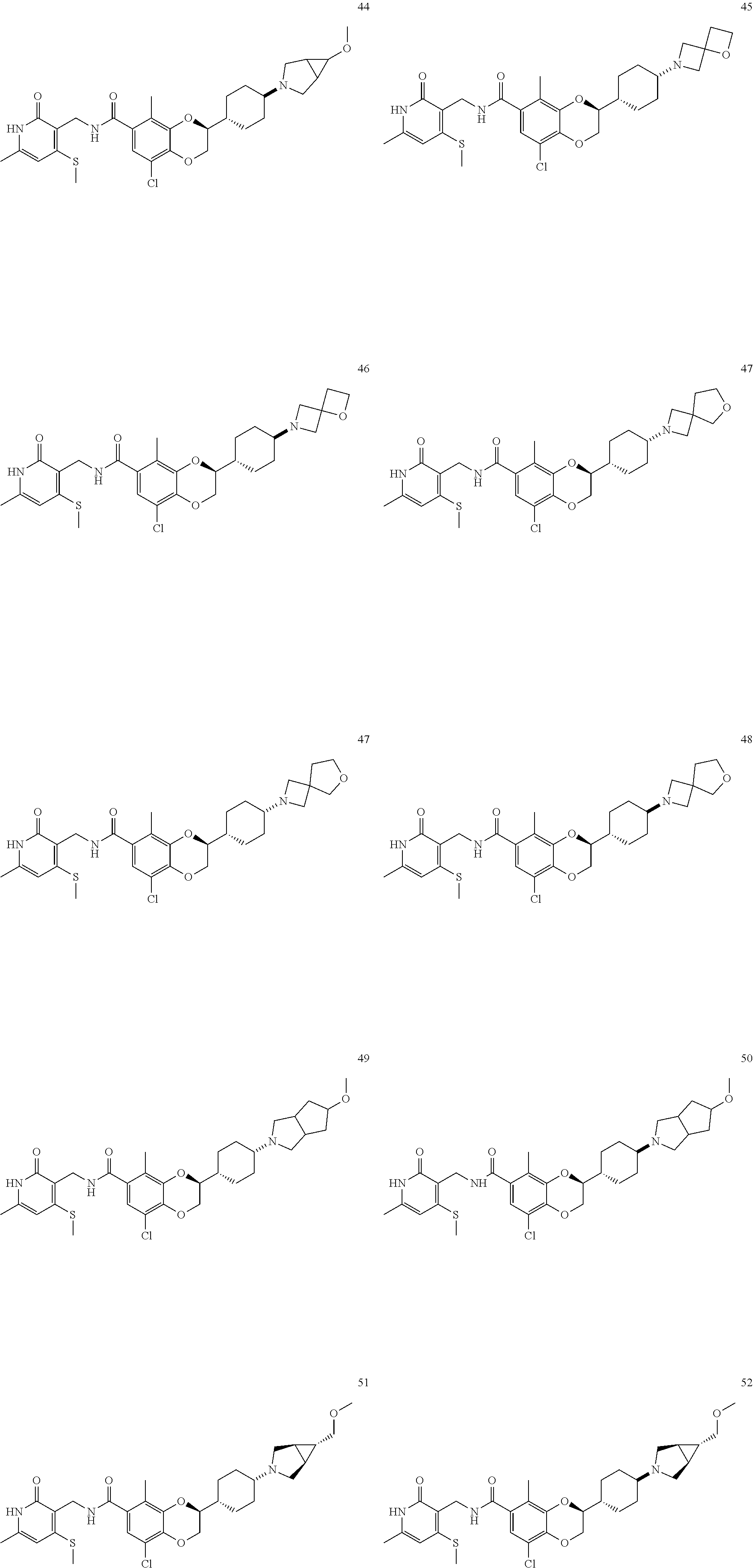

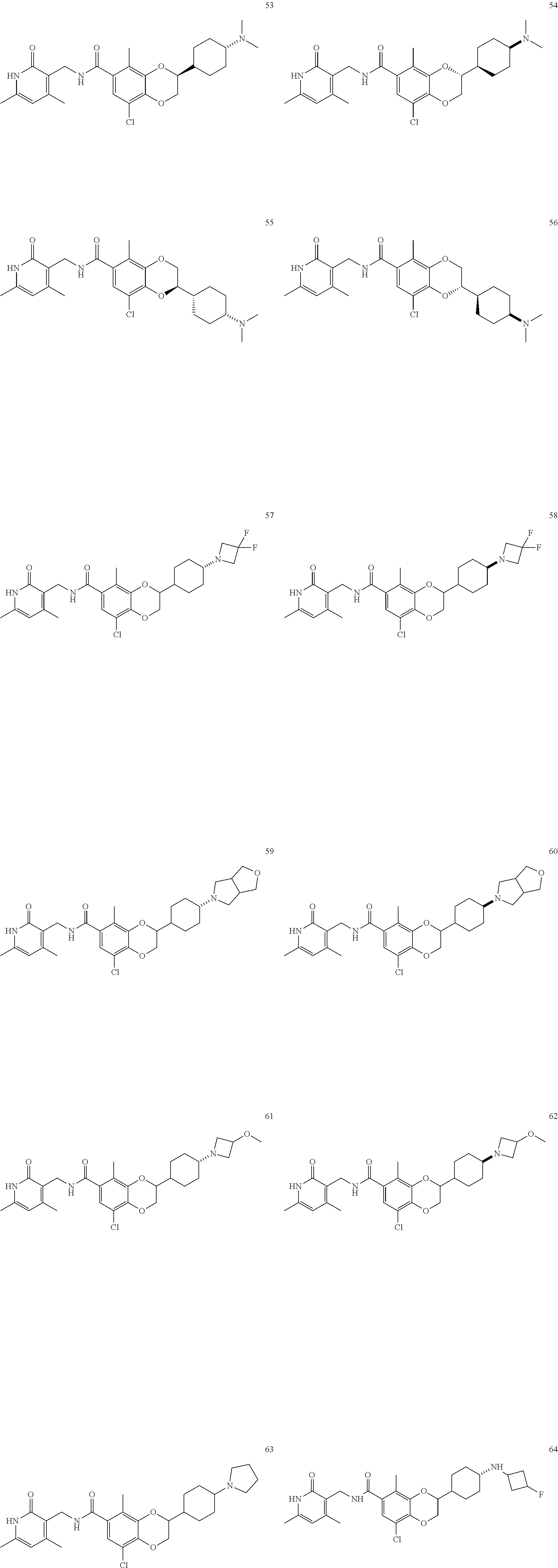

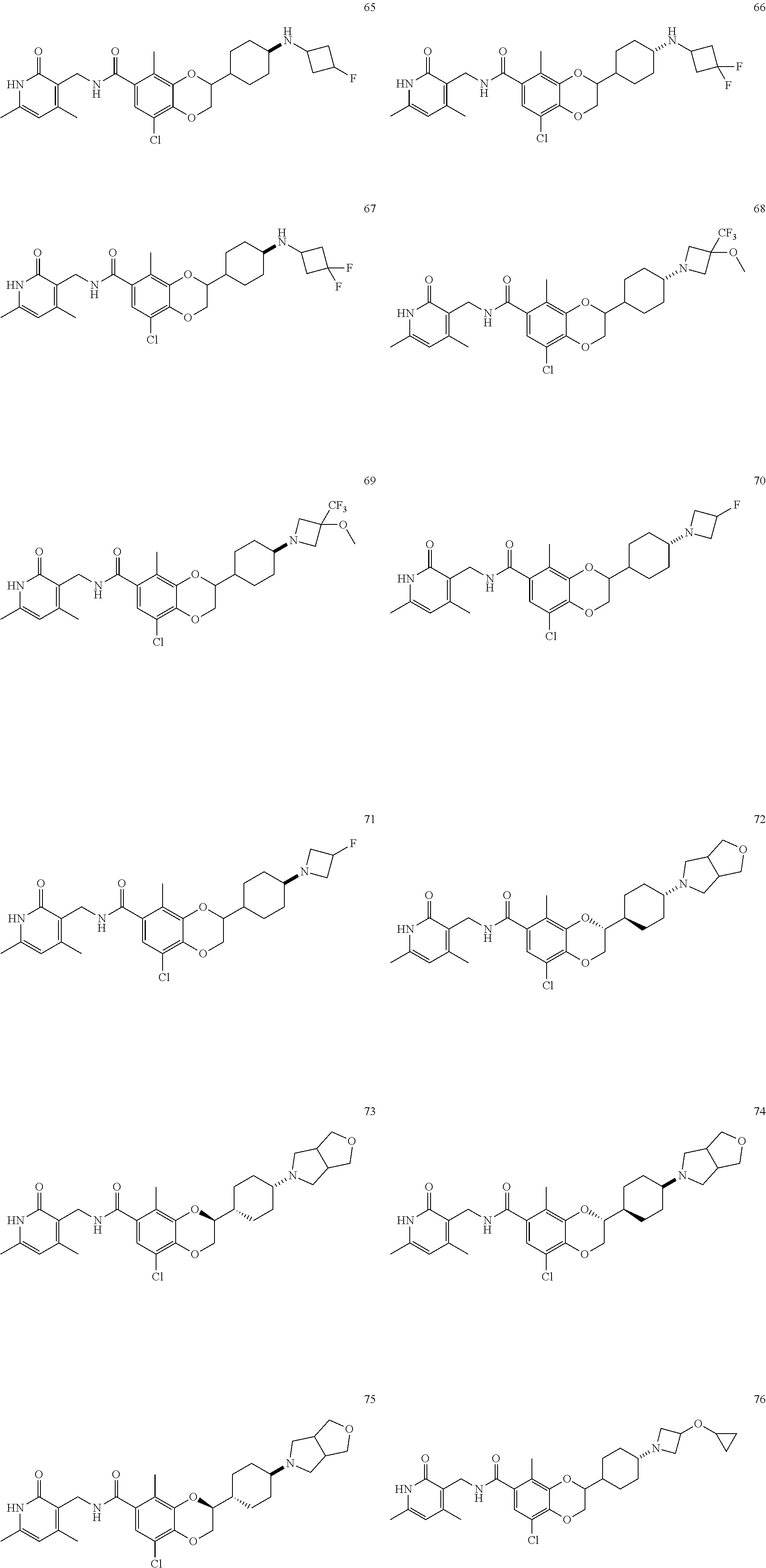

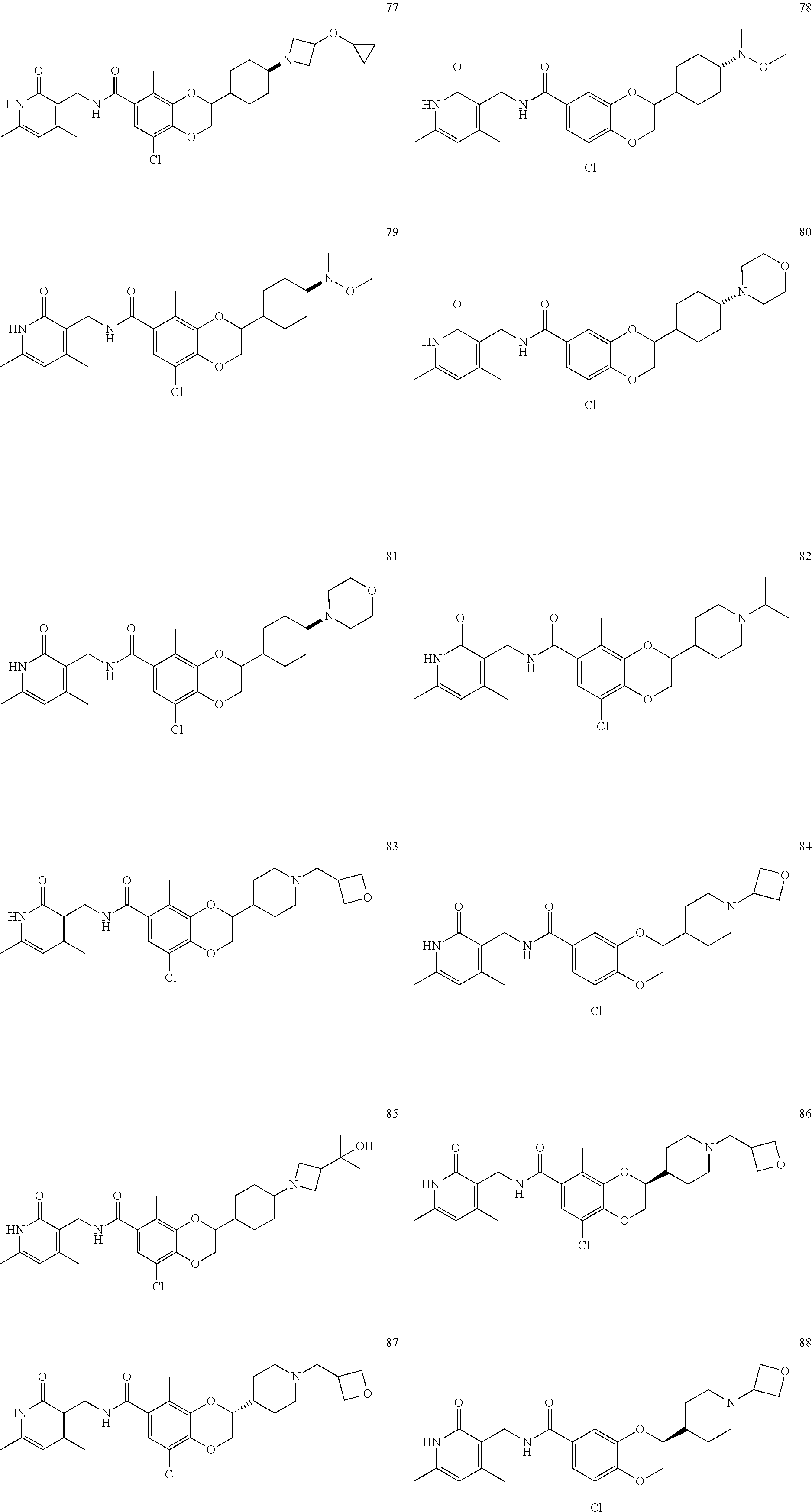

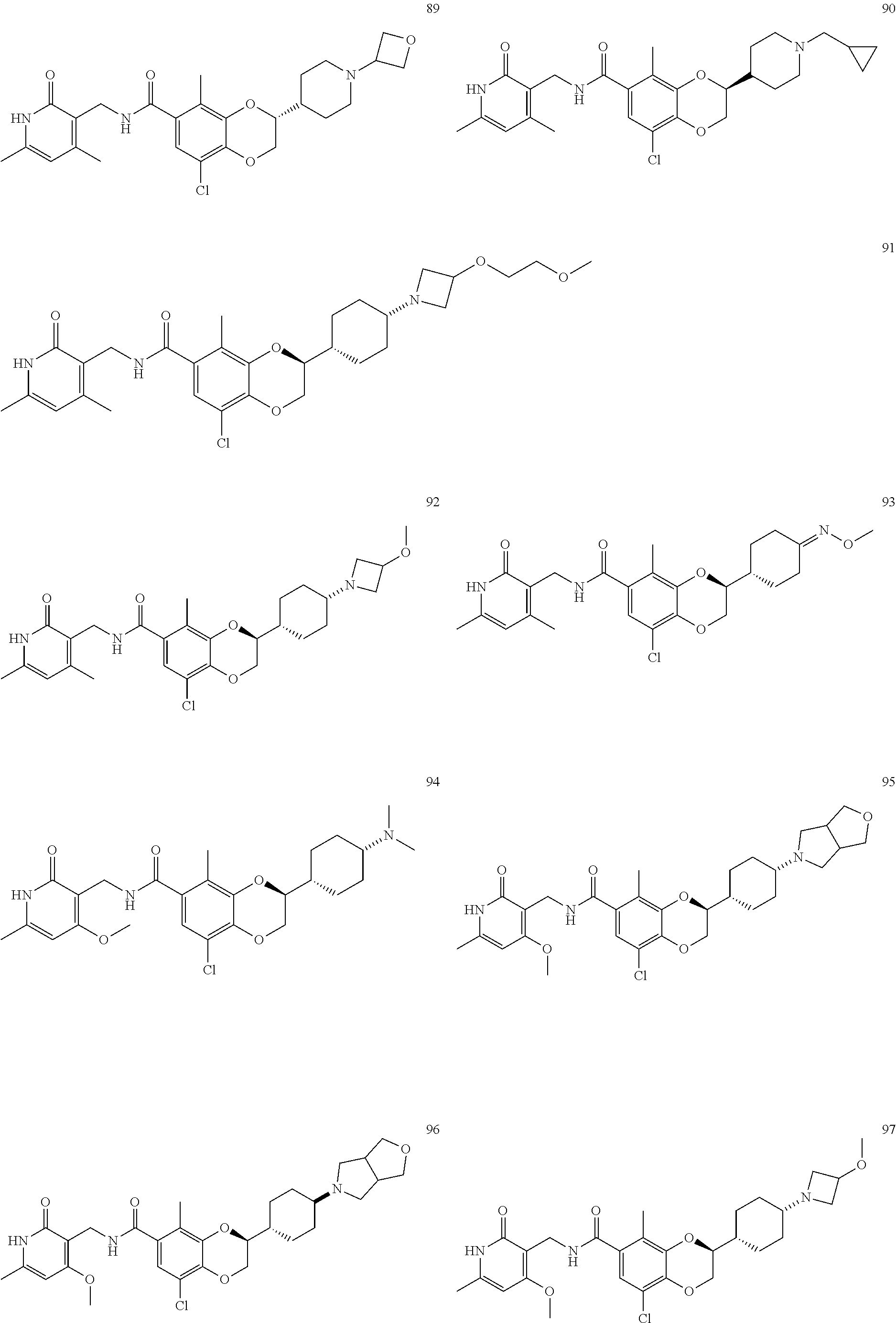

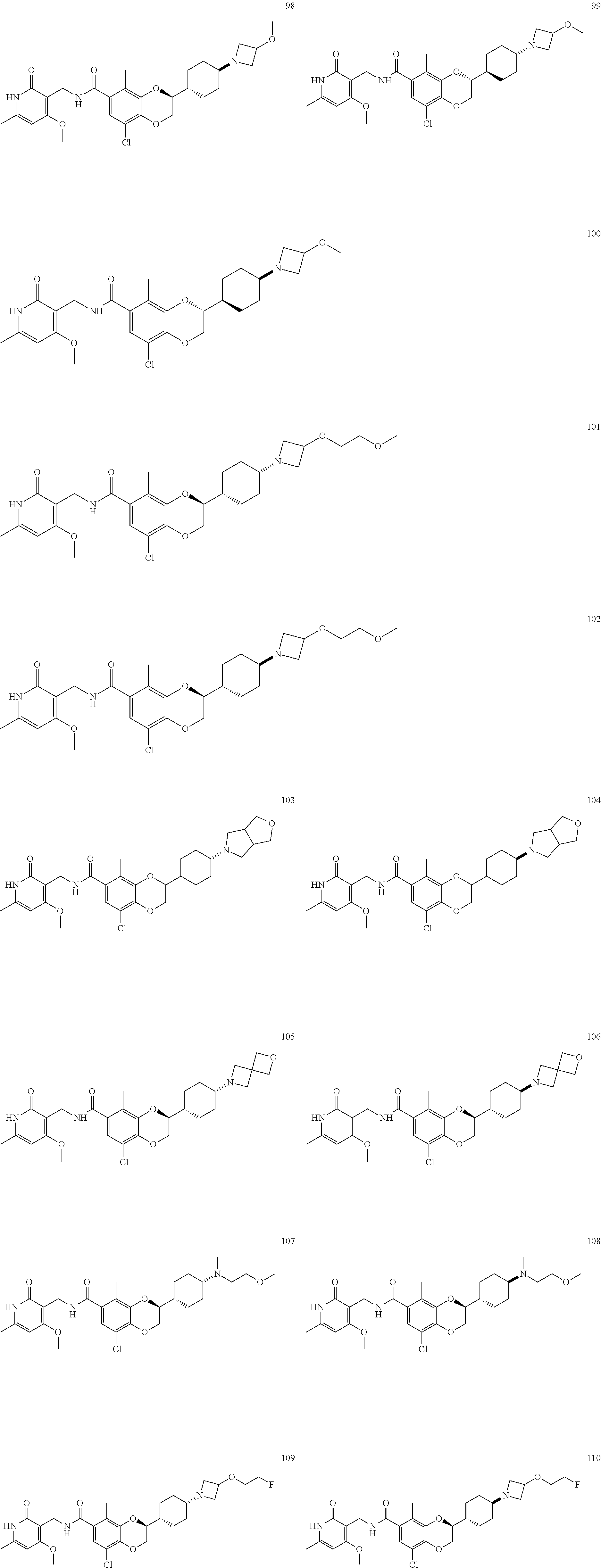

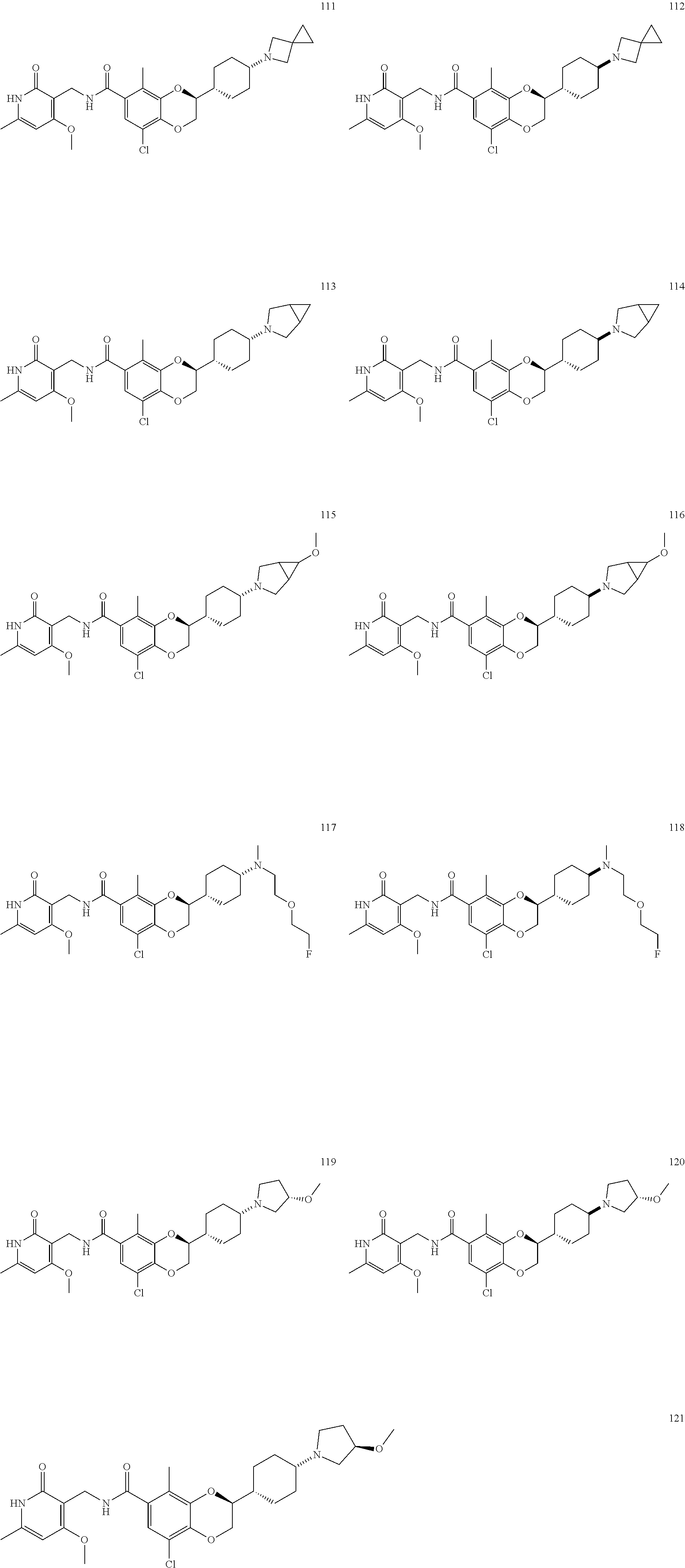

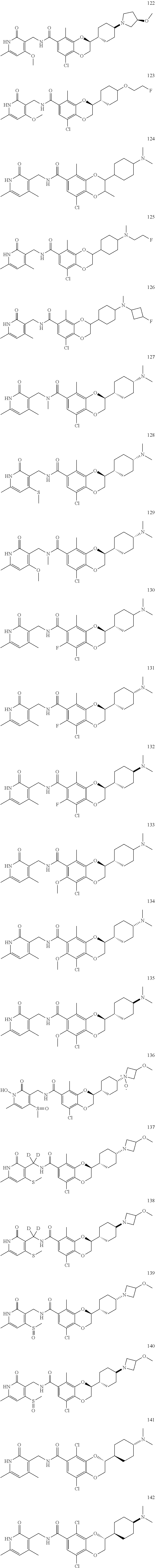

[chemical structure]

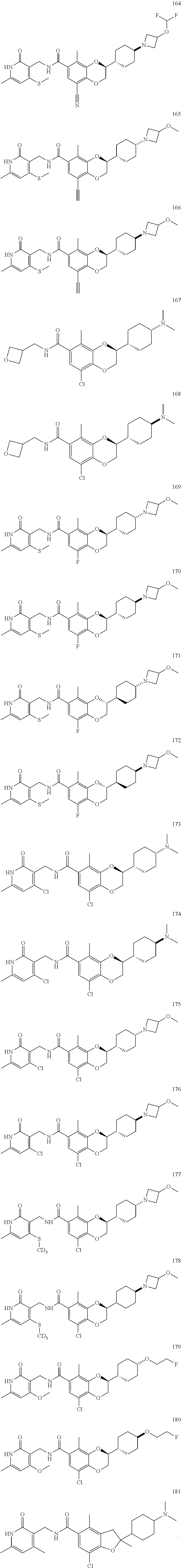

19. The compound, or a deuterated compound thereof, or a stereoisomer thereof, or a pharmaceutically acceptable salt thereof according to claim 1, wherein
the compound represented by Formula I or Formula II is specifically:

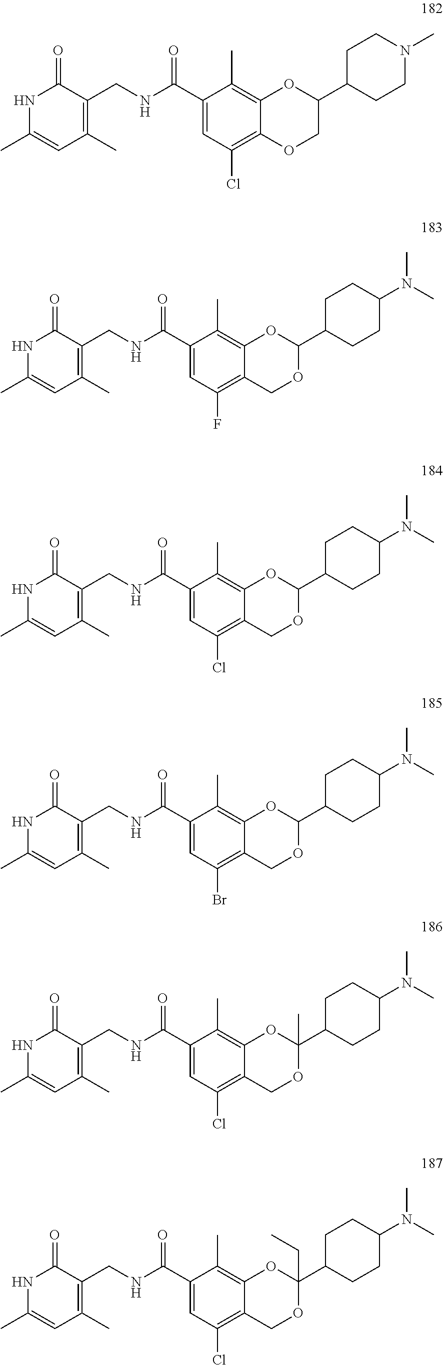

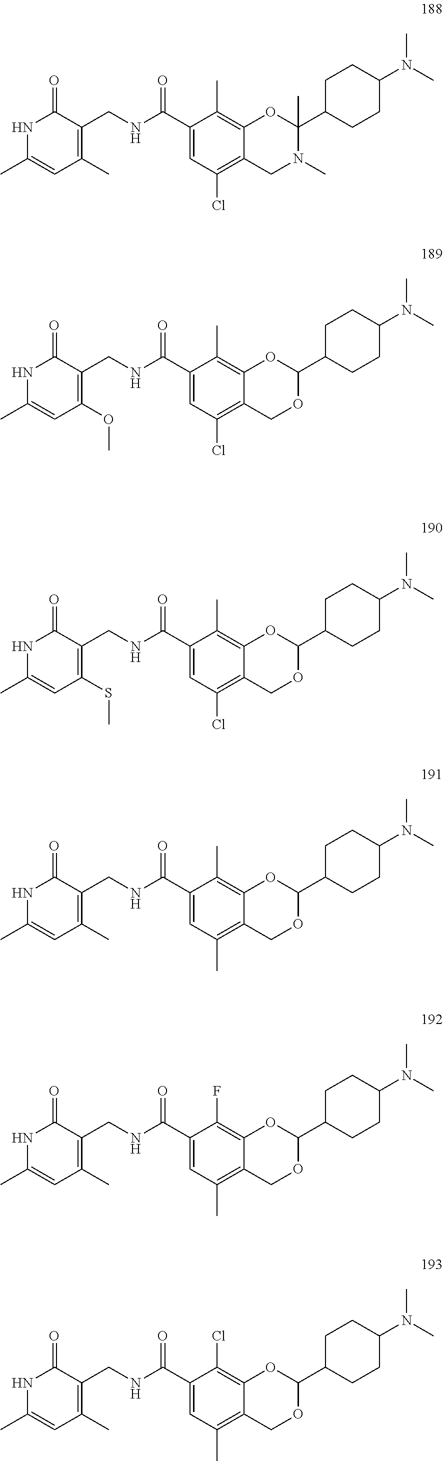

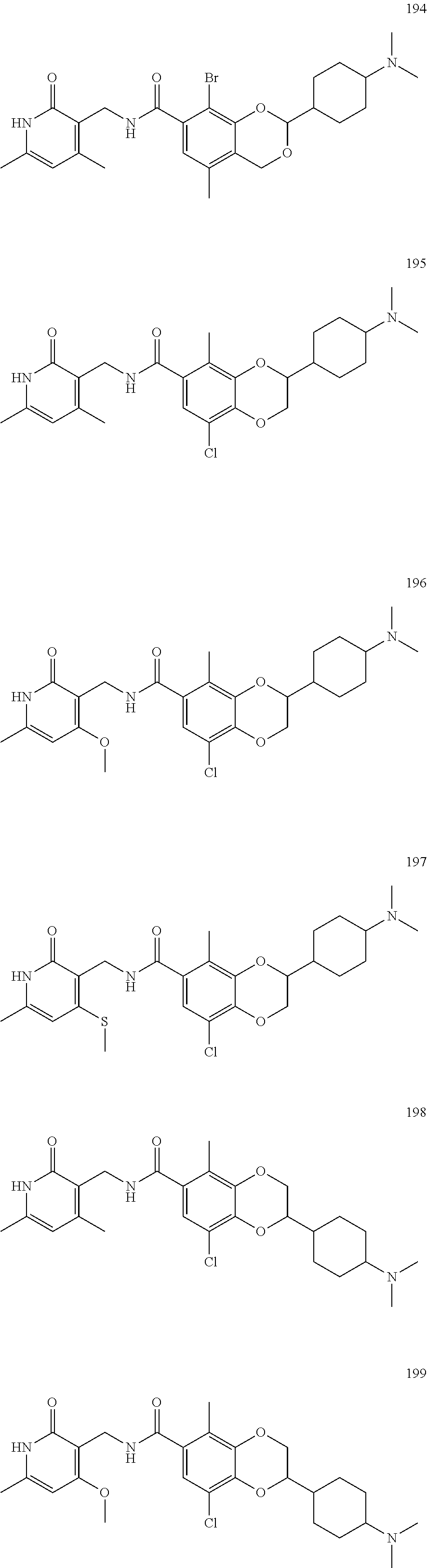

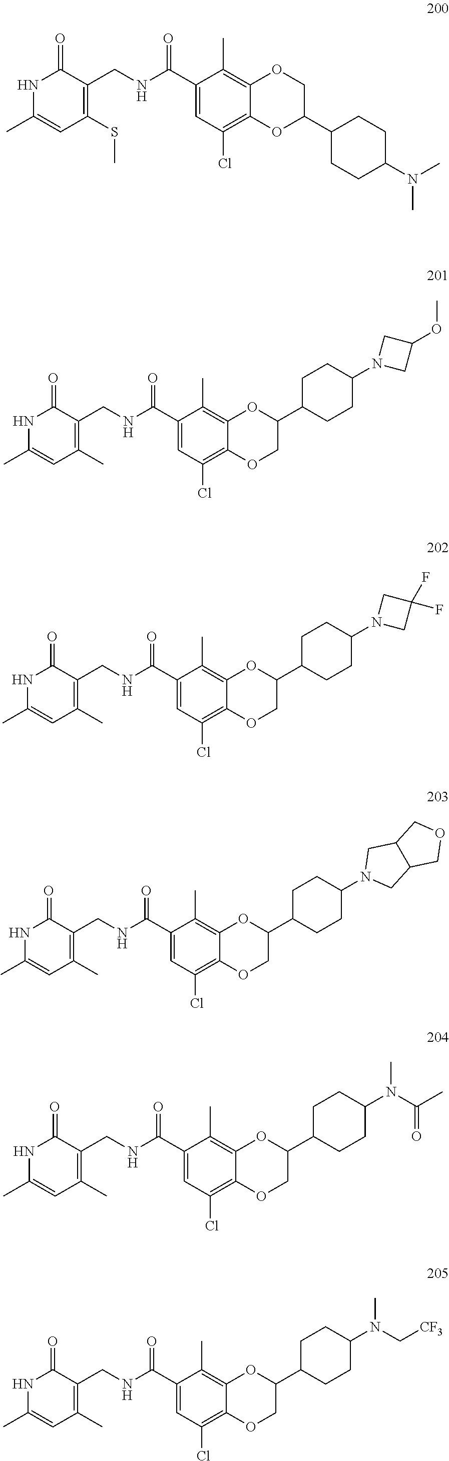

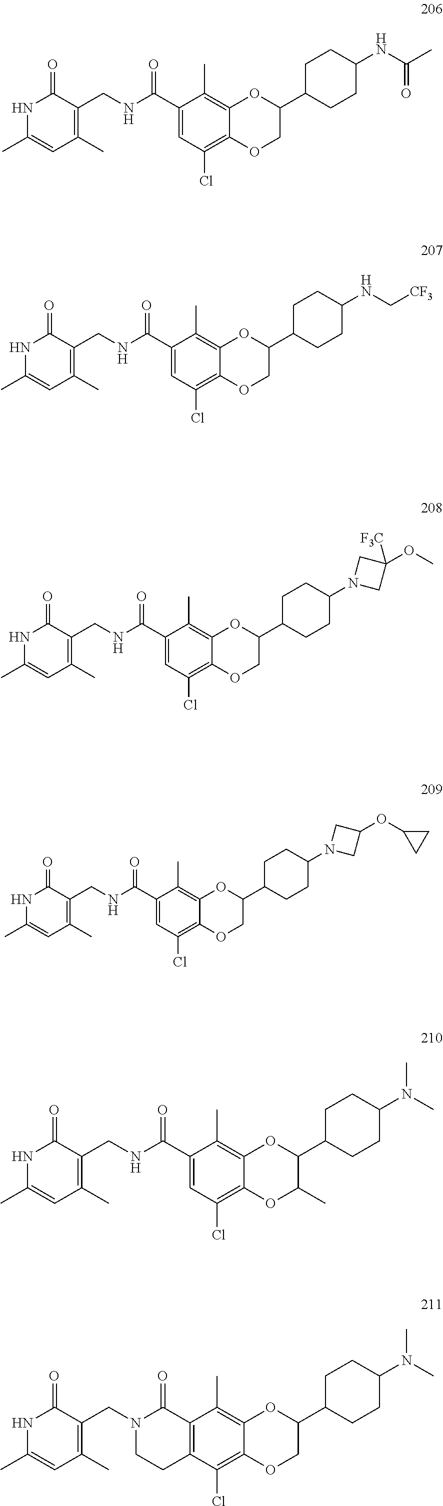

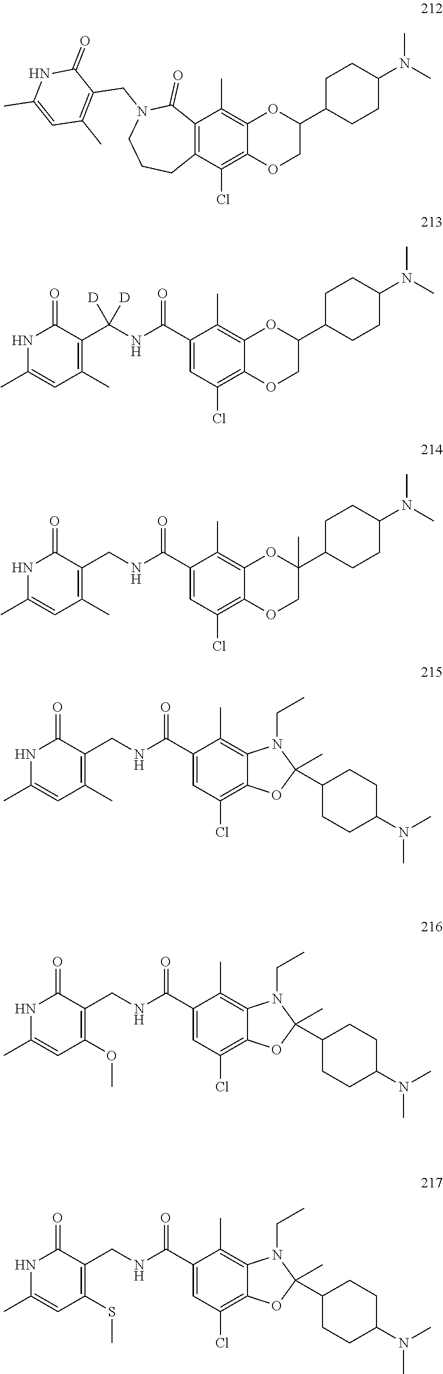

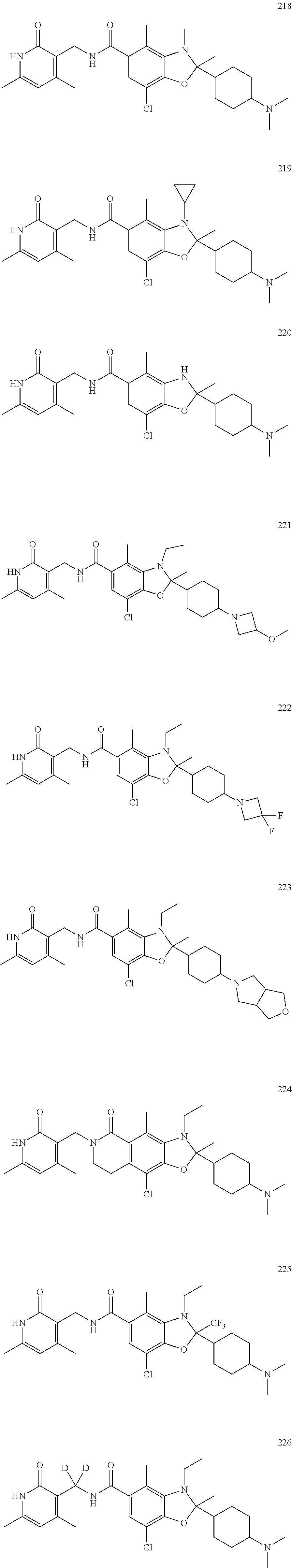

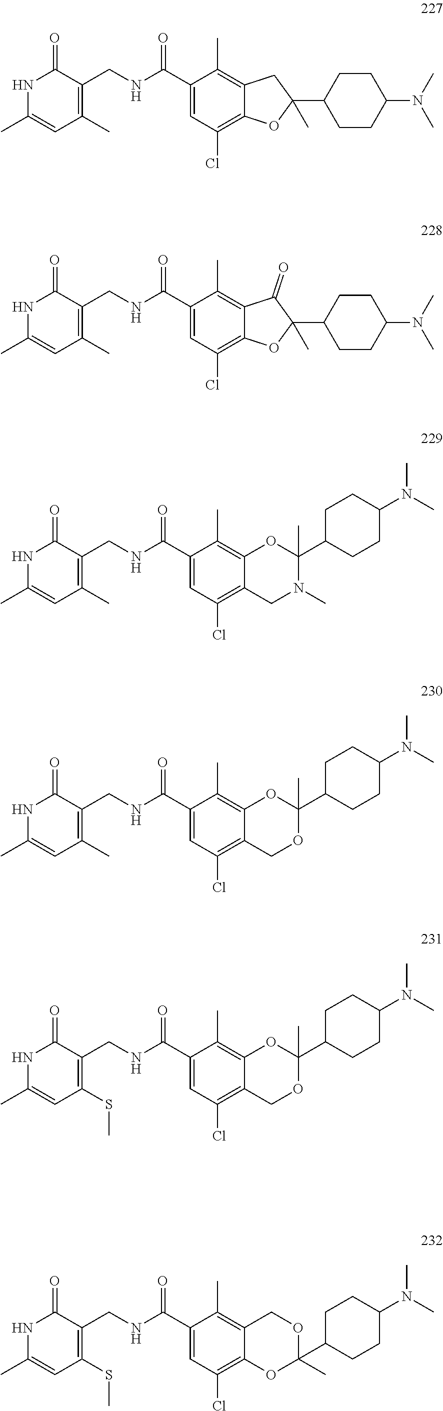

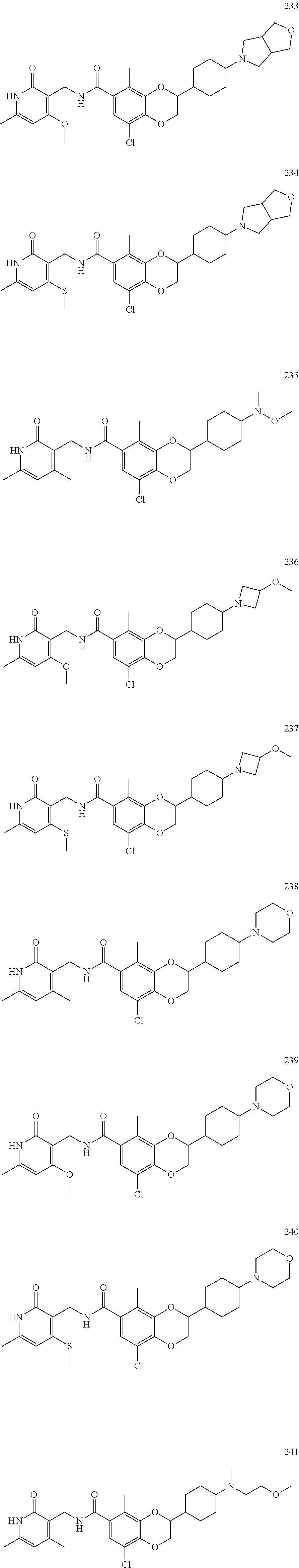

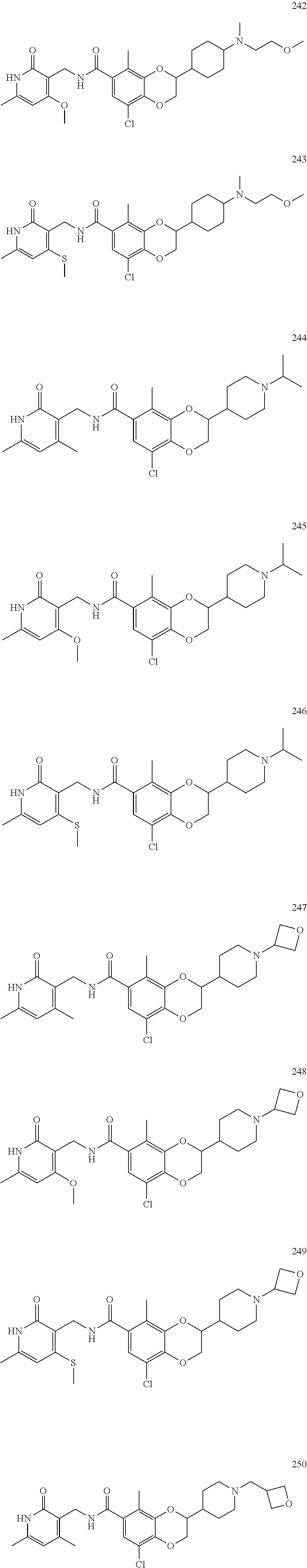

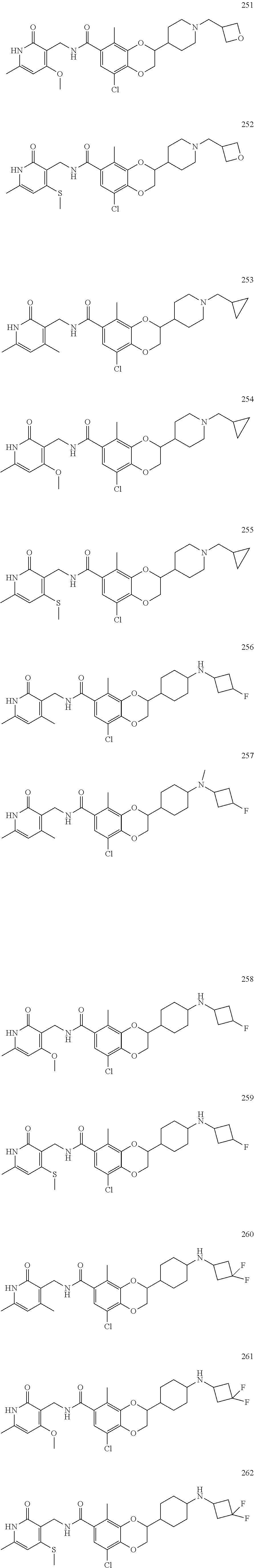

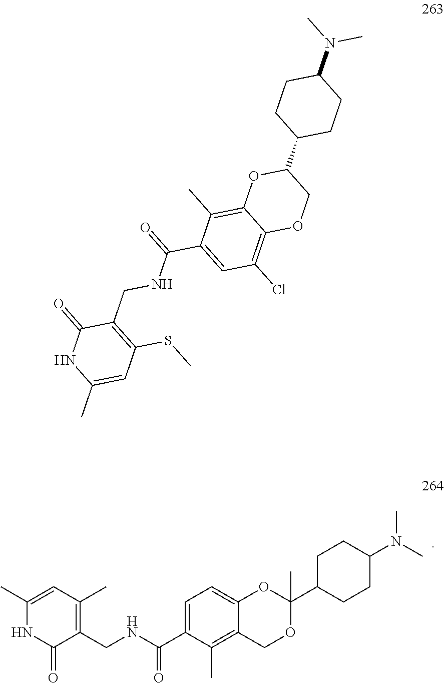

20. An intermediate compound shown as follows:

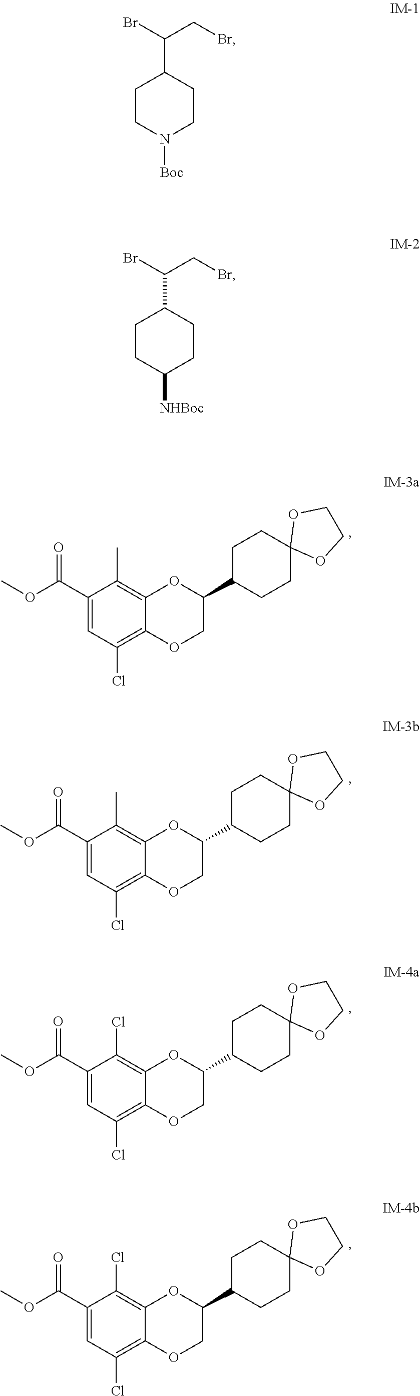

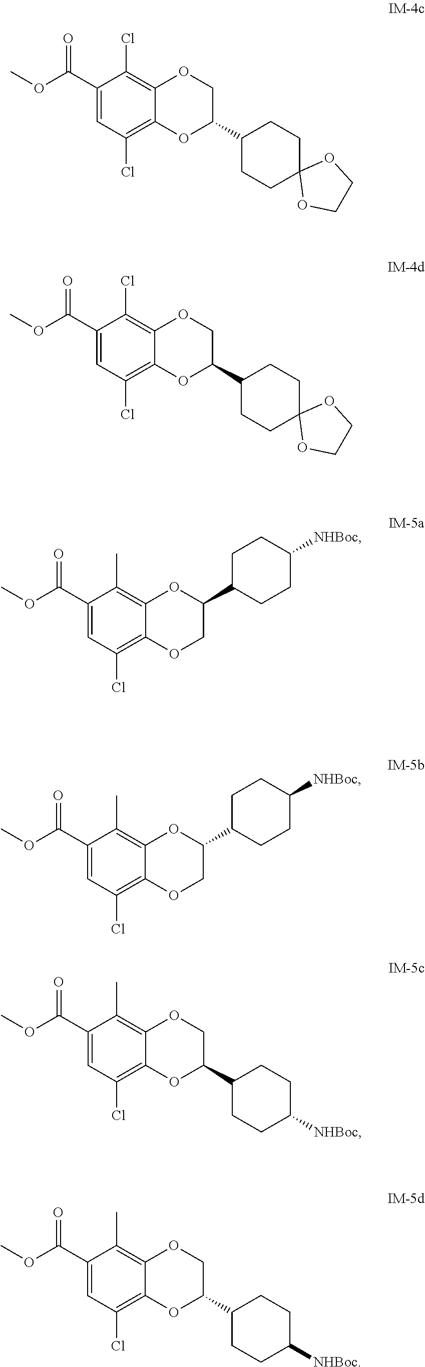

21. A method of preparing a medicament for treating an EZH1/2-mediated disease, comprising incorporating the compound according to claim 1, or a deuterated compound thereof, or a stereoisomer thereof, or a pharmaceutically acceptable salt thereof.

22. The method use according to claim 21, wherein the EZH1/2-mediated disease is one or more selected from diseases related to inflammation, autoimmune disease, infectious disease, cancer, and precancerous syndrome.

23. A pharmaceutical composition, which is a formulation prepared with the compound of claim 1, or a deuterated compound thereof, or a stereoisomer thereof, or a stereoisomer thereof, or a pharmaceutically acceptable salt thereof, as the pharmaceutically active ingredient, together with pharmaceutically acceptable excipients.
